(19) United States
(12) Patent Application Publication
(10) Pub. No.: US 2024/0200104 A1
(43) Pub. Date: Jun. 20, 2024

(54) LTR TRANSPOSON COMPOSITIONS AND METHODS

(71) Applicant: Flagship Pioneering Innovations VI, LLC, Cambridge, MA (US)

(72) Inventors: Robert James Citorik, Somerville, MA (US); William Edward Salomon, West Roxbury, MA (US); Zi Jun Wang, Arlington, MA (US); Jacob Rosenblum Rubens, Cambridge, MA (US); Benjamin Harris Weinberg, Cambridge, MA (US)

(21) Appl. No.: 18/282,644

(22) PCT Filed: Mar. 18, 2022
(86) PCT No.: PCT/US2022/020899

§ 371 Date: Sep. 18, 2023

Related U.S. Application Data
(60) Provisional application No. 63/163,532, filed on Mar. 19, 2021.

(51) Int. Cl. C12N 15/90; C12N 9/12; C12N 15/86

(52) U.S. Cl. CPC C12N 15/907; C12N 9/1276; C12N 15/86; C12Y 207/07049; C12N 2740/10022; C12N 2740/10023; C12N 2740/10043; C12N 2800/90

(57) ABSTRACT

Methods and compositions for altering a genome at one or more locations in a host cell, tissue, or subject are disclosed.

### CROSS-REFERENCE TO RELATED APPLICATIONS

This application claims the benefit of U.S. Provisional Application No. 63/163,532, filed Mar. 19, 2021. The contents of the aforementioned application are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Mar. 17, 2022, is named V2065-7016WO_SL.txt and is 662,130 bytes in size.

### BACKGROUND

Integration of a nucleic acid of interest into a genome occurs at low frequency and with little site specificity, in the absence of a specialized protein to promote the insertion event. Some existing approaches, like CRISPR/Cas9, are more suited for small edits and are less effective at integrating longer sequences. Other existing approaches, like Cre/loxP, require a first step of inserting a loxP site into the genome and then a second step of inserting a sequence of interest into the loxP site. There is a need in the art for improved proteins for inserting sequences of interest into a genome.

### SUMMARY OF THE INVENTION

This disclosure relates to novel compositions, systems and methods for altering a genome at one or more locations in a host cell, tissue or subject, in vivo or in vitro. In particular, the invention features compositions, systems and methods for the introduction of exogenous genetic elements into a host genome. The systems described herein typically include a template RNA comprising a pair of long terminal repeats (LTRs) flanking a heterologous object sequence (e.g., encoding a therapeutic effector), which can be introduced into a target cell with a structural polypeptide domain and a reverse transcriptase polypeptide domain, or nucleic acid molecules encoding same. Inside the cell, the template RNA and reverse transcriptase polypeptide domain can be enclosed within a proteinaceous exterior (e.g., a capsid), e.g., to form a virus-like particle (VLP). The reverse transcriptase polypeptide domain can then generate a template DNA from the template RNA. The resultant template DNA can then be integrated into the genome of the cell, e.g., by an integrase from a retrovirus or a retrotransposon, e.g., an LTR retrotransposon. Additionally described here are integration-deficient systems for providing an extrachromosomal DNA molecule to a host cell that does not undergo genomic integration. Thus, this disclosure provides systems capable of producing therapeutic DNA in a host cell, e.g., DNA encoding a therapeutic protein, by reverse transcription of an RNA template comprising LTRs, wherein the therapeutic DNA is optionally integrated into the host genome.

Features of the compositions or methods can include one or more of the following enumerated embodiments.
1. A system for modifying DNA comprising:
   a) a template RNA comprising a first long terminal repeat (LTR), a second LTR, a heterologous object sequence encoding a therapeutic effector, positioned between the first LTR and the second LTR, and optionally a primer binding site (PBS); or a DNA molecule encoding the template RNA;
   b) an LTR retrotransposon structural polypeptide domain (e.g., gag, e.g., a viral capsid (CA) protein), or a nucleic acid molecule encoding the structural polypeptide domain; and
   c) an LTR retrotransposon reverse transcriptase polypeptide domain (e.g., pol) capable of reverse transcribing the template RNA, thereby producing a template DNA, or a nucleic acid molecule encoding the reverse transcriptase polypeptide domain.
2. A system for modifying DNA comprising:
   a) a template RNA comprising a first LTR, a second LTR, and a heterologous object sequence encoding a therapeutic effector, positioned between the first LTR and the second LTR and optionally a primer binding site (PBS); or a DNA molecule encoding the template RNA;
   b) a retroviral structural polypeptide domain (e.g., gag), or a nucleic acid molecule encoding the structural polypeptide domain;
   c) a retroviral reverse transcriptase polypeptide domain (e.g., pol) capable of reverse transcribing the template RNA, thereby producing a template DNA, or a nucleic acid molecule encoding the reverse transcriptase polypeptide domain; and
   the system comprises neither an envelope polypeptide domain (e.g., a retroviral envelope polypeptide domain, e.g., a lentiviral envelope polypeptide domain) nor a nucleic acid molecule encoding the envelope polypeptide domain.
3. The system of embodiment 1 or 2, wherein the nucleic acid molecule of b), c), or of both of b) and c) are RNA.
4. A cell-free system for modifying DNA comprising:
   a) a template RNA comprising a first LTR, a second LTR, and a heterologous object sequence encoding a therapeutic effector, positioned between the first LTR and the second LTR and optionally a primer binding site (PBS); or a DNA molecule encoding the template RNA;
   b) a first RNA encoding a retroviral structural polypeptide domain (e.g., gag);
   c) a second RNA encoding a retroviral reverse transcriptase polypeptide domain (e.g., pol) capable of reverse transcribing the template RNA, thereby producing a template DNA, or a nucleic acid molecule encoding the reverse transcriptase polypeptide domain; and
   wherein the first RNA sequence and the second RNA sequence are optionally part of the same nucleic acid molecule.
5. The system of embodiment 4, wherein the system comprises neither an envelope polypeptide domain nor a nucleic acid molecule encoding the envelope polypeptide domain.
6. The system of any of the preceding embodiments, wherein the structural polypeptide domain (e.g., gag) comprises a mutation relative to a corresponding wild type structural polypeptide domain.
7. The system of any of the preceding embodiments, wherein the mutation in the structural polypeptide domain alters or decreases the cytoplasmic membrane localization of a component of the structural polypeptide domain (e.g., the gag protein, matrix protein, capsid protein, or nucleocapsid protein).
8. The system of any of the preceding embodiments, wherein the mutation in the structural polypeptide domain alters the intracellular localization of a component of the structural polypeptide domain (e.g., the gag protein, matrix protein, capsid protein, or nucleocapsid protein) to be cytoplasmic or at the endoplasmic reticulum.
9. The system of any of the preceding embodiments, wherein the mutation in the structural polypeptide domain reduces (e.g., eliminates) myristoylation of the structural polypeptide domain.
10. The system of any of the preceding embodiments, wherein the structural polypeptide domain comprises a matrix protein domain (e.g., a retroviral matrix protein domain).
11. The system of embodiment 10, wherein the matrix protein is encoded as a separate polypeptide from a further structural polypeptide domain (e.g., a capsid protein and/or a nucleocapsid protein).
12. The system of embodiment 10, wherein the matrix protein is encoded as part of the polypeptide as a further structural polypeptide domain (e.g., a capsid protein and/or a nucleocapsid protein).
13. The system of any of the preceding embodiments, wherein the structural polypeptide domain does not comprise a retroviral matrix protein domain.
14. The system of any of the preceding embodiments, wherein the structural polypeptide domain comprises a capsid protein domain (e.g., a retroviral capsid protein domain).
15. The system of embodiment 10, wherein the capsid protein is encoded as a separate polypeptide from a further structural polypeptide domain (e.g., a matrix protein and/or a nucleocapsid protein).
16. The system of embodiment 10, wherein the capsid protein is encoded as part of the polypeptide as a further structural polypeptide domain (e.g., a matrix protein and/or a nucleocapsid protein).
17. The system of any of the preceding embodiments, wherein the structural polypeptide domain does not comprise a retroviral capsid protein domain.
18. The system of any of the preceding embodiments, wherein the structural polypeptide domain comprises a nucleocapsid protein domain (e.g., a retroviral nucleocapsid protein domain).
19. The system of embodiment 10, wherein the nucleocapsid protein is encoded as a separate polypeptide from a further structural polypeptide domain (e.g., a matrix protein and/or a capsid protein).
20. The system of embodiment 10, wherein the nucleocapsid protein is encoded as part of the polypeptide as a further structural polypeptide domain (e.g., a matrix protein and/or a capsid protein).
21. The system of any of the preceding embodiments, wherein the structural polypeptide domain does not comprise a retroviral nucleocapsid protein domain.
22. The system of any of the preceding embodiments, wherein the reverse transcriptase polypeptide domain comprises a reverse transcriptase domain.
23. The system of embodiment 10, wherein the reverse transcriptase polypeptide domain is encoded as a separate polypeptide from a further polypeptide domain (e.g., an integrase protein, protease protein, dUTPase protein, viral accessory protein (e.g., vpr, vif, vpu, tat, rev, and/or nef), and/or ribonuclease H (RNase H) domain).
24. The system of embodiment 10, wherein the reverse transcriptase polypeptide domain is encoded as part of the polypeptide as a second polypeptide domain (e.g., an integrase protein, protease protein, dUTPase protein, viral accessory protein (e.g., vpr, vif, vpu, tat, rev, and/or nef), and/or ribonuclease H (RNase H) domain).
25. The system of any of the preceding embodiments, wherein the reverse transcriptase polypeptide domain comprises an integrase domain.
26. The system of any of the preceding embodiments, wherein the reverse transcriptase polypeptide domain comprises a protease domain.
27. The system of any of the preceding embodiments, wherein the reverse transcriptase polypeptide domain comprises a chromodomain.
28. The system of any of the preceding embodiments, wherein the reverse transcriptase polypeptide domain does not comprise a chromodomain.
29. A template RNA comprising:
   a first retrotransposon LTR,
   a second retrotransposon LTR,
   a heterologous object sequence encoding a therapeutic effector, positioned between the first LTR and the second LTR, and optionally, a primer binding site (PBS).
30. A DNA molecule encoding the template RNA of embodiment 29.
31. A method of delivering a heterologous object sequence to a target cell, comprising:
   a) introducing into the target cell (e.g., contacting the target cell with) a template RNA comprising a first LTR, a second LTR, and a heterologous object sequence encoding a therapeutic effector, positioned between the first LTR and the second LTR, and optionally a primer binding site (PBS); and
   b) introducing into the target cell (e.g., contacting the target cell with) an LTR retrotransposon structural polypeptide domain (e.g., gag), or a nucleic acid molecule encoding the structural polypeptide domain, and an LTR retrotransposon reverse transcriptase polypeptide domain (e.g., pol) capable of reverse transcribing the template RNA, thereby producing a template DNA, or a nucleic acid molecule encoding the reverse transcriptase polypeptide domain; and
   c) incubating the target cell under conditions suitable for production of the template DNA.
32. A method of delivering a heterologous object sequence to a target cell, comprising:
   a) introducing into the target cell (e.g., contacting the target cell with) a template RNA comprising a first LTR, a second LTR, and a heterologous object sequence encoding a therapeutic effector, positioned between the first LTR and the second LTR, and optionally a primer binding site (PBS); and
   b) contacting the target cell with a first RNA encoding a retroviral structural polypeptide domain (e.g., gag) and a second RNA encoding a retroviral reverse transcriptase polypeptide domain (e.g., pol) capable of reverse transcribing the template RNA, thereby producing a template DNA, wherein the first RNA and the second RNA are optionally part of the same RNA molecule, and
   c) incubating the target cell under conditions suitable for production of the template DNA.
33. The method of embodiment 32, wherein the first RNA and the second RNA overlap in sequence, e.g., wherein the coding region of the first RNA overlaps with the coding region of the second RNA.
34. A method of delivering a heterologous object sequence to a target cell, comprising:
   a) introducing into the target cell (e.g., contacting the target cell with) a template RNA comprising a first LTR, a second LTR, and a heterologous object sequence encoding a therapeutic effector, positioned between the first LTR and the second LTR, and optionally a primer binding site (PBS); and
   b) introducing into the target cell (e.g., contacting the target cell with) a retroviral structural polypeptide domain (e.g., gag), or a nucleic acid molecule encoding the structural polypeptide domain and a retroviral reverse transcriptase polypeptide domain (e.g., pol) capable of reverse transcribing the template RNA, thereby producing a template DNA, or a nucleic acid molecule encoding the reverse transcriptase polypeptide domain; and
   c) incubating the target cell under conditions suitable for production of the template DNA;
   wherein the method does not comprise introducing into the target cell either of an envelope polypeptide domain or a nucleic acid molecule encoding the envelope polypeptide domain.
35. A method of delivering a heterologous object sequence to a target cell of a patient in need thereof (e.g., in vivo or ex vivo delivery), comprising:
   a) introducing into the target cell (e.g., contacting the target cell with) a template RNA comprising a first LTR, a second LTR, and a heterologous object sequence encoding a therapeutic effector, positioned between the first LTR and the second LTR, and optionally a primer binding site (PBS); and
   b) contacting the target cell with a first polynucleotide encoding a retroviral structural polypeptide domain (e.g., gag), and a second polynucleotide encoding retroviral reverse transcriptase polypeptide domain (e.g., pol) capable of reverse transcribing the template RNA, thereby producing a template DNA, wherein the first polynucleotide and the second polynucleotide are optionally part of the same polynucleotide molecule; and
   c) incubating the target cell under conditions suitable for production of the template DNA.
36. The method of embodiment 35, wherein the target cell comprises neither an envelope polypeptide domain heterologous to the target cell nor a nucleic acid molecule encoding the envelope polypeptide domain.
37. The method of any of embodiments 31-36, wherein the method results in integration of the heterologous object sequence into the genome of the target cell.
38. The method of any of embodiments 31-37, wherein the method results in integration of the heterologous object sequence into a specific site within the genome of the target cell.
39. The method of any of embodiments 31-38, wherein the method results in integration of the heterologous object sequence into a random site within the genome of the target cell.
40. The method of any of embodiments 31-39, wherein the method results in integration of the heterologous object sequence preferentially a site having one or more (e.g., 1, 2, 3, 4, 5, 6, 7, 8, 9, 10, 11, 12, 13, 14, 15, 16, or all 17) of the following characteristics:
   (i) about 1 kb upstream of a gene transcribed by RNA pol III;
   (ii) about 2-3 kb (e.g., about 2, 2.5, or 3 kb) upstream of a gene transcribed by RNA pol III;
   (iii) comprises a silent mating locus;
   (iv) positioned within 10, 20, 30, 40, 50, 60, 70, 80, 90, 100, 500, or 1000 bp of a telomere;
   (v) within a promoter, e.g., a promoter for a gene transcribed by RNA pol II;
   (vi) within heterochromatin;
   (vii) within an enhancer;
   (viii) within a transcriptional start site;
   (ix) within a gene-rich region of a chromosome;
   (x) within a chromosomal region proximal to the nuclear periphery;
   (xi) within a nucleosome-free region;
   (xii) within a site hypersensitive to DNAse I;
   (xiii) located about 40-150 bp (e.g., about 40, 50, 51, 52, 53, 54, 60, 70, 80, 90, 100, 110, 120, 130, 140, or 150 bp of a tRNA coding region);
   (xiv) within an exon;
   (xv) within an intron;
   (xvi) within a gene (e.g., having a parallel orientation to the gene or having an antiparallel orientation to the gene); and/or
   (xvii) within a region into which one or more of the following retrotransposons and/or retroviruses is capable of integrating: Ty1, Ty3, Ty5, Tf1, Maggy, MLV, HIV, or PFV.
41. The method of any of embodiments 31-40, wherein the integration of the heterologous object sequence into the genome of the target cell results in one or more duplications at the integration site, e.g., duplications of 4-6 (e.g., 4, 5, or 6) nucleotides in length.
42. The method of any of embodiments 31-41, wherein the target cell is a human cell.
43. The method of any of embodiments 31-42, wherein the method results in production of an episome comprising the heterologous object sequence.
44. The method of embodiment 43, wherein the episome replicates in the target cell
45. The method of embodiment 43 or 44, wherein the episome comprises an origin of replication, e.g., a mammalian origin of replication, e.g., a human origin of replication.
46. The method of any of embodiments 43-45, wherein the episome does not replicate in the target cell
47. The method of any of embodiments 43-46, wherein the episome comprises one or two LTR sequences (e.g., comprises exactly one or exactly two LTR sequences).
48. The method of any of embodiments 43-47, wherein the episome is formed by circularization of the template DNA, e.g., using endogenous machinery of the target cell, e.g., using non-homologous end joining, homologous recombination (e.g., by strand invasion or single strand annealing), closure of intermediate products of reverse transcription, auto-integration, or ligation.
49. The method of any of embodiments 31-48, wherein the method results in production of an episome comprising the heterologous object sequence (thereby producing an episomal heterologous object sequence) and in integration of the heterologous object sequence into the genome of the target cell (thereby producing an integrated heterologous object sequence).
50. The method of embodiment 49, wherein the number of copies of the episomal heterologous object sequence is greater than the number of copies of integrated heterologous object sequence, e.g., by at least 2, 3, 4, 5, 6, 7, 8, 9, 10, 15, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000-fold.
51. The method of embodiment 49, wherein the number of copies of the integrated heterologous object sequence is greater than the number of copies of episomal heterologous object sequence, e.g., by at least 2, 3, 4, 5, 6, 7, 8, 9, 10, 15, 20, 25, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000-fold.
52. The method of any of embodiments 31-51, wherein the method results in integration of the first LTR and the second LTR into the genome of the target cell.
53. The method of embodiment 52, wherein the first LTR and the second LTR flank the heterologous object sequence after integration into the genome of the target cell.
54. The method of any of embodiments 31-53, wherein the method results in formation of a VLP in the target cell, wherein the VLP comprises: the template RNA, the structural polypeptide domain, and the reverse transcriptase polypeptide domain.
55. The method of embodiment 54, wherein the VLP further comprises one or more (e.g., 1, 2, 3, 4, 5, 6, or all 7) of: a matrix protein, nucleocapsid protein, capsid protein, reverse transcriptase protein, RNase H, protease, and integrase, e.g., of a retrovirus (e.g., a lentivirus) or a retrotransposon.
56. The method of embodiment 54, wherein the structural polypeptide domain encloses the template RNA and the reverse transcriptase polypeptide domain.
57. The method of any of embodiments 54-56, wherein the VLP enters the nucleus of the target cell, e.g., via a nuclear pore or via the endoplasmic reticulum.
58. The method of any of embodiments 54-57, wherein the VLP is initially localized to the cytoplasm.
59. The method of any of embodiments 54-58, wherein the VLP is initially localized to the endoplasmic reticulum (e.g., at the membrane of the endoplasmic reticulum).
60. The method of any of embodiments 54-59, wherein the template DNA is imported into the nucleus of the target cell and the capsid protein of the VLP is not imported into the nucleus of the target cell.
61. The method of embodiment 60, wherein the template DNA is injected into the nucleus of the target cell from the capsid protein of the VLP.
62. The method of any of embodiments 31-61, wherein the method results in formation of a PIC in the target cell, wherein the PIC comprises: the template DNA, the structural polypeptide domain, and the reverse transcriptase polypeptide domain.
63. The method of embodiment 62, wherein the structural polypeptide domain (e.g., a capsid protein) encloses the template DNA and the reverse transcriptase polypeptide domain.
64. The method of any of embodiments 31-63, wherein the method results in formation of an intracisternal particle (IAP) or an intracytoplasmic A-type particle (ICAP) in the target cell, e.g., in the cytoplasm or in the endoplasmic reticulum.
65. The method of any of embodiments 31-64, wherein the template RNA, the nucleic acid molecule encoding the structural polypeptide domain, and/or the nucleic acid molecule encoding the reverse transcriptase polypeptide domain are introduced into the target cell as RNA molecules (e.g., mRNAs).
66. The method of any of embodiments 31-65, wherein the template RNA, the nucleic acid molecule encoding the structural polypeptide domain, and/or the nucleic acid molecule encoding the reverse transcriptase polypeptide domain are introduced into the target cell as DNA molecules (e.g., episomes).
67. The method of any of embodiments 31-66, wherein the nucleic acid molecule encoding the structural polypeptide domain, and/or the nucleic acid molecule encoding the reverse transcriptase polypeptide domain are translated in the target cell, thereby producing the structural polypeptide domain and/or the reverse transcriptase polypeptide domain.
68. The system, template RNA, DNA molecule, or method of any of the preceding embodiments, wherein the template RNA is not part of the same nucleic acid molecule as the nucleic acid molecule encoding the structural polypeptide domain.
69. The system, template RNA, DNA molecule, or method of any of the preceding embodiments, wherein the template RNA is not part of the same nucleic acid molecule as the nucleic acid molecule encoding the reverse transcriptase polypeptide domain.
70. The system, template RNA, DNA molecule, or method of any of the preceding embodiments, wherein the template RNA is not part of the same nucleic acid molecule as either of the nucleic acid molecule encoding the structural polypeptide domain and the nucleic acid molecule encoding the reverse transcriptase polypeptide domain.
71. The system, template RNA, DNA molecule, or method of any of the preceding embodiments, wherein the structural polypeptide domain and the reverse transcriptase polypeptide domain are encoded on the same nucleic acid.
72. The system, template RNA, DNA molecule, or method of any of the preceding embodiments, wherein the structural polypeptide domain and the reverse transcriptase polypeptide domain are encoded on different nucleic acids.
73. The system, template RNA, DNA molecule, or method of any of the preceding embodiments, wherein the template RNA comprises a plurality of LTRs (e.g., exactly two LTRs).
74. The system, template RNA, DNA molecule, or method of any of the preceding embodiments, wherein the plurality of LTRs comprised in the template RNA share at least 75%, 80%, 85%, 90%, 95%, 96%, 97%, 98%, or 99% sequence identity.
75. The system, template RNA, DNA molecule, or method of any of the preceding embodiments, wherein the sequences of the plurality of LTRs comprised in the template RNA differ by no more than 1, 2, 3, 4, 5, 6, 7, 8, 9, 10, 20, 30, 40, 50, 60, 70, 80, 90, 100, 150, 200, 250, 300, 400, 500, 600, 700, 800, 900, or 1000 nucleotides.
76. The system, template RNA, DNA molecule, or method of any of the preceding embodiments, wherein one or more (e.g., both) of the LTRs comprised in the template RNA are each at least 200, 300, 400, 500, 600, 700, 800, 900, 1000, 1100, 1200, 1300, or 1400 nucleotides in length.
77. The system, template RNA, DNA molecule, or method of any of the preceding embodiments, wherein one or more (e.g., both) of the LTRs comprised in the template RNA are about 200-300, 300-400, 400-500, 500-600, 600-700, 700-800, 800-900, 900-1000, 1000-1100, 1100-1200, 1200-1300, or 1300-1400 nucleotides in length.
78. The system, template RNA, DNA molecule, or method of any of the preceding embodiments, wherein one or more (e.g., both) of the LTRs comprised in the template RNA comprises a U3 region (e.g., having a length of about 200-300, 300-400, 400-500, 500-600, 600-700, 700-800, 800-900, 900-1000, 1000-1100, or 1100-1200 nucleotides).
79. The system, template RNA, DNA molecule, or method of embodiment 78, wherein the U3 region is capable of being reverse transcribed by the reverse transcriptase polypeptide domain, e.g., to form a portion of the template DNA (e.g., a 3′ portion of the template DNA).
80. The system, template RNA, DNA molecule, or method of embodiment 78 or 79, wherein the U3 region comprises a deletion relative to a wild-type U3 region (e.g., a deletion of at least 10, 20, 30, 40, 50, 60, 70, 80, 90, 100, 200, 300, or 400 nucleotides).
81. The system, template RNA, DNA molecule, or method of any of the preceding embodiments, wherein one or more (e.g., both) of the LTRs comprised in the template RNA comprise a repeated region.
82. The system, template RNA, DNA molecule, or method of embodiment 81, wherein the repeated region is capable of being reverse transcribed by the reverse transcriptase polypeptide domain, e.g., to form a portion of the template DNA.
83. The system, template RNA, DNA molecule, or method of any of the preceding embodiments, wherein one or more (e.g., both) of the LTRs comprised in the template RNA comprises a U5 region (e.g., having a length of about 75-100, 100-125, 125-150, 150-175, 175-200, 200-225, or 225-250 nucleotides).
84. The system, template RNA, DNA molecule, or method of embodiment 83, wherein the U5 region is capable of being reverse transcribed by the reverse transcriptase polypeptide domain, e.g., to form a portion of the template DNA (e.g., a 5′ portion of the template DNA).
85. The system, template RNA, DNA molecule, or method of any of the preceding embodiments, wherein one or more (e.g., both) of the LTRs comprised in the template RNA have reduced promoter and/or enhancer activity relative to a wild-type LTR (e.g., reduced by at least 10%, 20%, 30%, 40%, 50%, 60%, 70%, 80%, 85%, 90%, 95%, 96%, 97%, 98%, 99%, or 100%).
86. The system, template RNA, DNA molecule, or method of any of the preceding embodiments, wherein the plurality of LTRs comprised in the template RNA are identical.
87. The system, template RNA, DNA molecule, or method of any of the preceding embodiments, wherein the first LTR is at the 5′ end of the template RNA, or less than 100, 90, 80, 70, 60, 50, 40, 30, 20, 10, 9, 8, 7, 6, 5, 4, 3, or 2 nucleotides from the 5′ end of the template RNA.
88. The system, template RNA, DNA molecule, or method of any of the preceding embodiments, wherein the second LTR is at the 3′ end of the template RNA, or less than 2, 3, 4, 5, or 10 nucleotides from the 3′ end of the template RNA.
89. The system, template RNA, DNA molecule, or method of any of the preceding embodiments, wherein the polynucleotide (e.g., RNA) encoding the retroviral structural polypeptide domain does not comprise an LTR or does not comprise an LTR within 500 bp, 1 kb, 1.5 kb, or 2 kb of its coding region.
90. The system, template RNA, DNA molecule, or method of any of the preceding embodiments, wherein the polynucleotide (e.g., RNA) encoding the retroviral structural polypeptide domain does not comprise two LTRs or does not comprise two LTRs within 500 bp, 1 kb, 1.5 kb, or 2 kb of its coding region.
91. The system, template RNA, DNA molecule, or method of any of the preceding embodiments, wherein the polynucleotide (e.g., RNA) encoding the reverse transcriptase polypeptide domain does not comprise an LTR or does not comprise an LTR within 500 bp, 1 kb, 1.5 kb, or 2 kb of its coding region.
92. The system, template RNA, DNA molecule, or method of any of the preceding embodiments, wherein the polynucleotide (e.g., RNA) encoding the reverse transcriptase polypeptide domain does not comprise two LTRs or does not comprise two LTRs within 500 bp, 1 kb, 1.5 kb, or 2 kb of its coding region.
93. The system, template RNA, DNA molecule, or method of any of the preceding embodiments, wherein the reverse transcriptase polypeptide domain (e.g., pol) comprises integrase activity, e.g., encodes a viral integrase, e.g., having an integrase amino acid sequence as listed in Table H1 or H2.
94. The system, template RNA, DNA molecule, or method of any of the preceding embodiments, wherein the viral integrase specifically binds the first LTR and the second LTR.
95. The system, template RNA, DNA molecule, or method of any of the preceding embodiments, wherein the template RNA comprises a primer-binding site (PBS).
96. The system, template RNA, DNA molecule, or method of embodiment 95, wherein the PBS comprises the nucleic acid sequence of a PBS from an LTR retrotransposon or a retrovirus (e.g., a lentivirus, e.g., HIV), or a nucleic acid sequence having at least 75%, 80%, 85%, 90%, 95%, 96%, 97%, 98%, or 99% sequence identity thereto.
97. The system, template RNA, DNA molecule, or method of embodiment 95 or 96, wherein the PBS is positioned downstream of the first LTR.
98. The system, template RNA, DNA molecule, or method of any of embodiments 95-97, wherein the PBS is positioned upstream of the heterologous object sequence.
99. The system, template RNA, DNA molecule, or method of any of embodiments 95-98, wherein the PBS can bind to an RNA endogenous to the target cell, e.g., a tRNA, e.g., a lysyl tRNA.
100. The system, template RNA, DNA molecule, or method of any of the preceding embodiments, wherein the template RNA comprises a polypurine tract.
101. The system, template RNA, DNA molecule, or method of embodiment 100, wherein the polypurine tract comprises the nucleic acid sequence of a polypurine tract from an LTR retrotransposon or a retrovirus (e.g., a lentivirus, e.g., HIV), or a nucleic acid sequence having at least 75%, 80%, 85%, 90%, 95%, 96%, 97%, 98%, or 99% sequence identity thereto.
102. The system, template RNA, DNA molecule, or method of embodiment 100 or 101, wherein the polypurine tract is positioned downstream of the heterologous object sequence.
103. The system, template RNA, DNA molecule, or method of any of embodiments 100-102, wherein the polypurine tract is positioned upstream of the second LTR.
104. The system, template RNA, DNA molecule, or method of any of the preceding embodiments, wherein the template RNA comprises a promoter and/or an enhancer.
105. The system, template RNA, DNA molecule, or method of embodiment 104, wherein the promoter comprises the nucleic acid sequence of a promoter from an LTR retrotransposon or a retrovirus (e.g., a lentivirus, e.g., HIV), or a nucleic acid sequence having at least 75%, 80%, 85%, 90%, 95%, 96%, 97%, 98%, or 99% sequence identity thereto.
106. The system, template RNA, DNA molecule, or method of embodiment 104, wherein the promoter comprises the nucleic acid sequence of a promoter heterologous to an LTR retrotransposon or a retrovirus (e.g., a lentivirus, e.g., HIV), e.g., a constitutive promoter or a tissue-specific promoter.
107. The system, template RNA, DNA molecule, or method of any of embodiments 104-107, wherein the promoter is positioned downstream of the primer binding site.
108. The system, template RNA, DNA molecule, or method of any of embodiments 104-108, wherein the promoter is comprised by the heterologous object sequence.
109. The system, template RNA, DNA molecule, or method of any of the preceding embodiments, wherein the template RNA comprises an open reading frame, e.g., encoding a therapeutic effector comprised by the heterologous object sequence.
110. The system, template RNA, DNA molecule, or method of embodiment 109, wherein the open reading frame is positioned downstream of the promoter.
111. The system, template RNA, DNA molecule, or method of any of embodiments 109-110, wherein the open reading frame is positioned upstream of the polypurine tract.
112. The system, template RNA, DNA molecule, or method of any of embodiments 109-111, wherein the open reading frame is comprised in the heterologous object sequence.
113. The system, template RNA, DNA molecule, or method of any of the preceding embodiments, wherein the template RNA comprises a dimerization initiation signal.
114. The system, template RNA, DNA molecule, or method of embodiment 113, wherein the dimerization initiation signal is positioned downstream of the primer binding site.
115. The system, template RNA, DNA molecule, or method of any of the preceding embodiments, wherein the template RNA comprises a packaging signal (Psi).
116. The system, template RNA, DNA molecule, or method of any of the preceding embodiments, wherein the template RNA comprises a Rev-responsive element (RRE).
117. The system, template RNA, DNA molecule, or method of embodiment 115 or 116, wherein the Psi and/or RRE positioned downstream of the dimerization initiation signal.
118. The system, template RNA, DNA molecule, or method of embodiment 115 or 116, wherein the Psi and/or RRE positioned upstream of the heterologous object sequence.
119. The system, template RNA, DNA molecule, or method of any of the preceding embodiments, wherein the template RNA comprises a post-transcriptional regulatory element.
120. The system, template RNA, DNA molecule, or method of embodiment 119, wherein the post-transcriptional regulatory element is positioned downstream of the heterologous object sequence.
121. The system, template RNA, DNA molecule, or method of embodiment 119, wherein the post-transcriptional regulatory element is positioned upstream of the polypurine tract.
122. The system, template RNA, DNA molecule, or method of any of the preceding embodiments, wherein the template RNA comprises a gag gene, or a fragment thereof.
123. The system, template RNA, DNA molecule, or method of any of the preceding embodiments, wherein the template RNA comprises one or more non-canonical or modified ribonucleotides.
124. The system, template RNA, DNA molecule, or method of any of the preceding embodiments, wherein the nucleic acid molecule encoding the structural polypeptide domain comprises one or more non-canonical or modified ribonucleotides.
125. The system, template RNA, DNA molecule, or method of any of the preceding embodiments, wherein the nucleic acid molecule encoding the reverse transcriptase polypeptide domain comprise one or more non-canonical or modified ribonucleotides.
126. The system, template RNA, DNA molecule, or method of any of embodiments 123-125, wherein the modified ribonucleotides comprise chemically modified ribonucleotides.
127. The system, template RNA, DNA molecule, or method of any of the preceding embodiments, wherein the template RNA is a circular RNA.
128. The system, template RNA, DNA molecule, or method of any of the preceding embodiments, wherein the nucleic acid molecule encoding the structural polypeptide domain and/or the nucleic acid molecule encoding the reverse transcriptase polypeptide domain are circular RNAs.
129. The system, template RNA, DNA molecule, or method of any of the preceding embodiments, wherein the template RNA comprises a non-translated cap (e.g., a 5′ cap).
130. The system, template RNA, DNA molecule, or method of embodiment 129, wherein the non-translated cap comprises: a 5′ cap, e.g., a 5′ cap with cap-0, cap-1, or cap-2 structure, anti-reverse cap analog (ARCA) (m27.3′-OGP3G), GP3G (Unmethylated Cap Analog), m7GP3G (Monomethylated Cap Analog), m32.2.7GP3G (Trimethylated Cap Analog), a 7-methylguanosine cap (e.g., a 0-Me-m7G cap); a hypermethylated cap analog; an NAD+-derived cap analog (e.g., as described in Kiledjian, Trends in Cell Biology 28, 454-464 (2018)), a modified, e.g., biotinylated, cap analog (e.g., as described in Bednarek et al., Phil Trans R Soc B 373, 20180167 (2018)), or a cap as listed in Table M3.
131. The system, template RNA, DNA molecule, or method of any of the preceding embodiments, wherein the template RNA comprises a non-translated tail (e.g., a poly-A tail).
132. The system, template RNA, DNA molecule, or method of embodiment 131, wherein the non-translated tail comprises: a polyA tail, a 16-nucleotide long stem-loop structure flanked by unpaired 5 nucleotides (e.g., as described by Mannironi et al., Nucleic Acid Research 17, 9113-9126 (1989)), a triple-helical structure (e.g., as described by Brown et al., PNAS 109, 19202-19207 (2012)), a tRNA, Y RNA, or vault RNA structure (e.g., as described by Labno et al., Biochemica et Biophysica Acta 1863, 3125-3147 (2016)), or one or more deoxyribonucleotide triphosphates (dNTPs), 2′O-Methylated NTPs, or phosphorothioate-NTPs.
133. The system, template RNA, DNA molecule, or method of any of the preceding embodiments, wherein the nucleic acid molecule encoding the structural polypeptide domain and/or the nucleic acid molecule encoding the reverse transcriptase polypeptide domain comprises a non-translated cap (e.g., a 5′ cap).
134. The system, template RNA, DNA molecule, or method of any of the preceding embodiments, wherein the nucleic acid molecule encoding the structural polypeptide domain and/or the nucleic acid molecule encoding the reverse transcriptase polypeptide domain comprises a non-translated tail (e.g., a poly-A tail).
135. The system, template RNA, DNA molecule, or method of any of the preceding embodiments, wherein the template RNA is single stranded.
136. The system, template RNA, DNA molecule, or method of any of the preceding embodiments, wherein the nucleic acid molecule encoding the structural polypeptide domain and/or the nucleic acid molecule encoding the reverse transcriptase polypeptide domain are single stranded.
137. The system, template RNA, DNA molecule, or method of any of the preceding embodiments, wherein the nucleic acid molecule encoding the structural polypeptide domain comprises an internal ribosome entry site (IRES).
138. The system, template RNA, DNA molecule, or method of any of the preceding embodiments, wherein the nucleic acid molecule encoding the reverse transcriptase polypeptide domain comprises an internal ribosome entry site (IRES).
139. The system, template RNA, DNA molecule, or method of any of the preceding embodiments, wherein the nucleic acid molecule encoding the structural polypeptide domain and the reverse transcriptase polypeptide domain comprises an internal ribosome entry site (IRES), e.g., positioned between the sequence encoding the structural polypeptide domain and the sequence encoding the reverse transcriptase polypeptide domain.
140. The system, template RNA, DNA molecule, or method of any of the preceding embodiments, wherein the nucleic acid molecule encoding the structural polypeptide domain and the reverse transcriptase polypeptide domain comprises a small repetitive motif (e.g., comprising the nucleic acid sequence AAAAA), e.g., positioned between the sequence encoding the structural polypeptide domain and the sequence encoding the reverse transcriptase polypeptide domain.
141. The system, template RNA, DNA molecule, or method of any of the preceding embodiments, wherein the nucleic acid molecule encoding the structural polypeptide domain and the reverse transcriptase polypeptide domain can hybridize to a tRNA (e.g., a tRNA capable of ribosomal stalling and slippage).
142. The system, template RNA, DNA molecule, or method of any of the preceding embodiments, wherein the nucleic acid molecule encoding the structural polypeptide domain and the reverse transcriptase polypeptide domain does not comprise a nucleic acid sequence encoding a retroviral (e.g., lentiviral) env protein.
143. The system, template RNA, DNA molecule, or method of any of the preceding embodiments, wherein the nucleic acid molecule encoding the structural polypeptide domain and the reverse transcriptase polypeptide domain does not comprise a nucleic acid sequence encoding a retroviral (e.g., lentiviral) vif, vpr, vpu, and/or nef protein.
144. The system, template RNA, DNA molecule, or method of any of the preceding embodiments, wherein the nucleic acid molecule encoding the structural polypeptide domain and the reverse transcriptase polypeptide domain does not comprise a nucleic acid sequence encoding a retroviral (e.g., lentiviral) tat protein.
145. The system, template RNA, DNA molecule, or method of any of the preceding embodiments, wherein the template RNA encodes an intron.
146. The system, template RNA, DNA molecule, or method of embodiment 145, wherein the intron is positioned in the heterologous object sequence.
147. The system, template RNA, DNA molecule, or method of embodiment 145 or 146, wherein the intron is oriented parallel relative to the template RNA.
148. The system, template RNA, DNA molecule, or method of embodiment 145 or 146, wherein the intron is oriented antiparallel relative to the template RNA.
149. The system, template RNA, DNA molecule, or method of any of the preceding embodiments, wherein the template RNA comprises one or more elements from MusD, Gypsy/Ty3, Copia/Ty1, Bel/Pao, Morgane, BARE2, Large Retrotransposon Derivative (LARD), Terminal-repeat Retrotransposon in Miniature (TRIM), IAP, or ETn, or a functional fragment or variant thereof, or a nucleic acid sequence having at least 75%, 80%, 85%, 90%, 95%, 96%, 97%, 98%, or 99% sequence identity thereto.
150. The system, template RNA, DNA molecule, or method of any of the preceding embodiments, wherein the template RNA comprises one or more elements from a lentivirus (e.g., an HIV, e.g. HIV-1 or HIV-2), metavirus, pseudovirus, belpaovirus, betaretrovirus, picornavirus (e.g., enterovirus, e.g., enterovirus 71, coxsackievirus A16, or poliovirus), hepatovirus (e.g., a hepatitis virus, e.g., hepatitis A virus), calcivirus (e.g., norovirus or vesivirus), alphavirus (e.g., Semliki Forest virus, Sindbis virus, and Venezuelan equine encephalitis virus), flavivirus (e.g., Kunjin virus, yellow fever virus, West Nile virus, dengue virus, Zika virus, encephalitis virus, or hepacivirus, e.g., hepatitis C virus), coronavirus (e.g., murine hepatitis virus, SARS-CoV, or SARS-CoV-2), hepevirus (e.g., hepatitis E virus), reovirus, birnavirus (e.g., avibirnavirus), arenavirus, vesicular stomatitis virus, or a functional fragment or variant thereof, or a nucleic acid sequence having at least 75%, 80%, 85%, 90%, 95%, 96%, 97%, 98%, or 99% sequence identity thereto.
151. The system, template RNA, DNA molecule, or method of any of the preceding embodiments, wherein the template RNA comprises one or more elements from an endogenous retrovirus (e.g., an endogenous retrovirus in the human genome or a mammalian genome).
152. The system, template RNA, DNA molecule, or method of any of the preceding embodiments, wherein the template RNA, the structural polypeptide domain (or the nucleic acid molecule encoding the structural polypeptide domain), and the reverse transcriptase polypeptide domain (or the nucleic acid molecule encoding the reverse transcriptase polypeptide domain) are comprised in a lipid nanoparticle (LNP).
153. The system, template RNA, DNA molecule, or method of any of the preceding embodiments, wherein the template RNA is comprised in an LNP.
154. The system, template RNA, DNA molecule, or method of any of the preceding embodiments, wherein the structural polypeptide domain (or the nucleic acid molecule encoding the structural polypeptide domain) is comprised in an LNP.
155. The system, template RNA, DNA molecule, or method of any of the preceding embodiments, wherein the reverse transcriptase polypeptide domain (or the nucleic acid molecule encoding the reverse transcriptase polypeptide domain) is comprised in an LNP.
156. The system, template RNA, DNA molecule, or method of any of embodiments 152-155, wherein the template RNA, the structural polypeptide domain (or the nucleic acid molecule encoding the structural polypeptide domain), and the reverse transcriptase polypeptide domain (or the nucleic acid molecule encoding the reverse transcriptase polypeptide domain) are comprised in the same LNP.
157. The system, template RNA, DNA molecule, or method of any of embodiments 152-155, wherein the template RNA, the structural polypeptide domain (or the nucleic acid molecule encoding the structural polypeptide domain), and the reverse transcriptase polypeptide domain (or the nucleic acid molecule encoding the reverse transcriptase polypeptide domain) are comprised in different LNPs.
158. The system, template RNA, DNA molecule, or method of any of embodiments 152-155, wherein the template RNA and the structural polypeptide domain (or the nucleic acid molecule encoding the structural polypeptide domain) are comprised in different LNPs.
159. The system, template RNA, DNA molecule, or method of any of embodiments 152-155, wherein the template RNA and the reverse transcriptase polypeptide domain (or the nucleic acid molecule encoding the reverse transcriptase polypeptide domain) are comprised in different LNPs.
160. The system, template RNA, DNA molecule, or method of any of embodiments 152-155, wherein the structural polypeptide domain (or the nucleic acid molecule encoding the structural polypeptide domain), and the reverse transcriptase polypeptide domain (or the nucleic acid molecule encoding the reverse transcriptase polypeptide domain) are comprised in different LNPs.
161. The system, template RNA, DNA molecule, or method of any of the preceding embodiments, wherein the template RNA is produced by a process comprising:
   providing a precursor RNA that comprises a self-cleaving ribozyme and a region comprising a sequence of the template RNA, and
   incubating the precursor RNA under conditions that allow for self-cleavage,
   thereby producing the template RNA.
162. The system, template RNA, DNA molecule, or method of any of the preceding embodiments, wherein the template RNA is produced by a process comprising:
   providing a precursor RNA that comprises a region comprising a sequence of the template RNA and an oligonucleotide binding sequence,
   contacting the precursor RNA with an oligonucleotide that binds the oligonucleotide binding sequence,
   contacting the precursor RNA with RNaseH, and
   incubating the precursor RNA under conditions that allow RNAseH mediated cleavage,
   thereby producing the template RNA.
163. The system, template RNA, DNA molecule, or method of any of the preceding embodiments, wherein the structural polypeptide domain and the reverse transcriptase polypeptide domain are part of the same polypeptide.
164. The system, template RNA, DNA molecule, or method of any of the preceding embodiments, wherein the LTR retrotransposon structural polypeptide domain is a protein from MusD, Gypsy/Ty3, Copia/Ty1, Bel/Pao, Morgane, BARE2, Large Retrotransposon Derivative (LARD), Terminal-repeat Retrotransposon in Miniature (TRIM), IAP, or ETn, or a functional fragment or variant thereof, or a polypeptide having at least 75%, 80%, 85%, 90%, 95%, 96%, 97%, 98%, or 99% sequence identity thereto.
165. The system, template RNA, DNA molecule, or method of any of the preceding embodiments, wherein the LTR retrotransposon reverse transcriptase polypeptide domain is a protein from MusD, Gypsy/Ty3, Copia/Ty1, Bel/Pao, Morgane, BARE2, Large Retrotransposon Derivative (LARD), Terminal-repeat Retrotransposon in Miniature (TRIM), IAP, or ETn, or a functional fragment or variant thereof, or a polypeptide having at least 75%, 80%, 85%, 90%, 95%, 96%, 97%, 98%, or 99% sequence identity thereto.
166. The system, template RNA, DNA molecule, or method of any of the preceding embodiments, wherein the retroviral structural polypeptide domain is a protein from a lentivirus (e.g., an HIV, e.g. HIV-1 or HIV-2), metavirus, pseudovirus, belpaovirus, betaretrovirus, picornavirus (e.g., enterovirus, e.g., enterovirus 71, coxsackievirus A16, or poliovirus), hepatovirus (e.g., a hepatitis virus, e.g., hepatitis A virus), calcivirus (e.g., norovirus or vesivirus), alphavirus (e.g., Semliki Forest virus, Sindbis virus, and Venezuelan equine encephalitis virus), flavivirus (e.g., Kunjin virus, yellow fever virus, West Nile virus, dengue virus, Zika virus, encephalitis virus, or hepacivirus, e.g., hepatitis C virus), coronavirus (e.g., murine hepatitis virus, SARS-CoV, or SARS-CoV-2), hepevirus (e.g., hepatitis E virus), reovirus, birnavirus (e.g., avibirnavirus), arenavirus, vesicular stomatitis virus, or a functional fragment or variant thereof, or a polypeptide having at least 75%, 80%, 85%, 90%, 95%, 96%, 97%, 98%, or 99% sequence identity thereto.
167. The system, template RNA, DNA molecule, or method of any of the preceding embodiments, wherein the retroviral reverse transcriptase polypeptide domain is a protein from a lentivirus (e.g., an HIV, e.g. HIV-1 or HIV-2), metavirus, pseudovirus, belpaovirus, betaretrovirus, picornavirus (e.g., enterovirus, e.g., enterovirus 71, coxsackievirus A16, or poliovirus), hepatovirus (e.g., a hepatitis virus, e.g., hepatitis A virus), calcivirus (e.g., norovirus or vesivirus), alphavirus (e.g., Semliki Forest virus, Sindbis virus, and Venezuelan equine encephalitis virus), flavivirus (e.g., Kunjin virus, yellow fever virus, West Nile virus, dengue virus, Zika virus, encephalitis virus, or hepacivirus, e.g., hepatitis C virus), coronavirus (e.g., murine hepatitis virus, SARS-CoV, or SARS-CoV-2), hepevirus (e.g., hepatitis E virus), reovirus, birnavirus (e.g., avibirnavirus), arenavirus, vesicular stomatitis virus, or a functional fragment or variant thereof, or a polypeptide having at least 75%, 80%, 85%, 90%, 95%, 96%, 97%, 98%, or 99% sequence identity thereto.
168. The system, template RNA, DNA molecule, or method of any of the preceding embodiments, wherein the retroviral structural polypeptide domain is a protein encoded by an endogenous retrovirus (e.g., an endogenous retrovirus in the human genome).
169. The system, template RNA, DNA molecule, or method of any of the preceding embodiments, wherein the retroviral reverse transcriptase polypeptide domain is a protein encoded by an endogenous retrovirus (e.g., an endogenous retrovirus in the human genome).
170. The system, template RNA, DNA molecule, or method of any of the preceding embodiments, wherein the reverse transcriptase polypeptide domain is substantially unable to integrate the template DNA into a target DNA.
171. The system, template RNA, DNA molecule, or method of embodiment 170, wherein the reverse transcriptase polypeptide domain has reduced integrase activity, e.g., to at least 50%, 40%, 30%, 20%, 10%, 5%, 2%, or 1% of that of a corresponding wild-type sequence, e.g., as measured in an assay as described in Moldt et al. 2008 (BMC Biotechnol. 8:60; incorporated herein by reference).
172. The system, template RNA, DNA molecule, or method of embodiment 170 or 171, wherein the reverse transcriptase polypeptide domain comprises a mutation that reduces integrase activity, e.g., to at least 50%, 40%, 30%, 20%, 10%, 5%, 2%, or 1% of a corresponding wild-type sequence.
173. The system, template RNA, DNA molecule, or method of any of the preceding embodiments, wherein the system further comprises, or wherein the method further comprises contacting the cell with, an inhibitor (e.g., a small molecule inhibitor) of wild-type viral integrase activity.
174. The system, template RNA, DNA molecule, or method of any of the preceding embodiments, wherein the template RNA does not comprise a wild-type retroviral (e.g., lentiviral) attachment site at one or both ends.
175. The system, template RNA, DNA molecule, or method of any of the preceding embodiments, wherein the system that does not comprise a nucleic acid molecule encoding the envelope polypeptide domain comprises a nonfunctional fragment of an env gene, e.g., a fragment of less than 2000, 1500, 1000, 900, 800, 700, 600, 500, 400, 300, 200, 100, 50, 40, 30, 20, or 10 contiguous nucleotides.
176. The system, template RNA, DNA molecule, or method of any of the preceding embodiments, wherein the system that does not comprise a nucleic acid molecule encoding the envelope polypeptide domain comprises an env gene with a premature stop codon.
177. The system, template RNA, DNA molecule, or method of any of the preceding embodiments, wherein the system that does not comprise a nucleic acid molecule encoding the envelope polypeptide domain comprises a nonfunctional env gene, e.g., comprising less than 2000, 1500, 1000, 900, 800, 700, 600, 500, 400, 300, 200, 100, 50, 40, 30, 20, or 10 contiguous nucleotides from the sequence of a wild-type env gene.
178. The system, template RNA, DNA molecule, or method of any of the preceding embodiments, wherein the system that does not comprise a nucleic acid molecule encoding the envelope polypeptide domain the system does not comprise a functional env gene.
179. The system, template RNA, DNA molecule, or method of any of the preceding embodiments, wherein the target cell is a mammalian cell (e.g., a human cell).
180. The system, template RNA, DNA molecule, or method of any of the preceding embodiments, wherein the target cell is a primary cell.
181. The system, template RNA, DNA molecule, or method of any of the preceding embodiments, wherein the target cell is not immortalized.
182. The system, template RNA, DNA molecule, or method of any of the preceding embodiments, wherein the target cell is euploid.
183. The system, template RNA, DNA molecule, or method of any of the preceding embodiments, wherein the target cell is comprised in a subject (e.g., a patient, e.g., a human patient).
184. The system, template RNA, DNA molecule, or method of embodiment 183, wherein the template RNA, the nucleic acid molecule encoding the structural polypeptide domain, and/or the nucleic acid molecule encoding the reverse transcriptase polypeptide domain are introduced into the target cell via a lipid nanoparticle.
185. The system, template RNA, DNA molecule, or method of any of the preceding embodiments, wherein the target cell is obtained from a subject (e.g., a patient, e.g., a human patient), e.g., wherein the target cell is a autologous to the subject.
186. The system, template RNA, DNA molecule, or method of embodiment 185, wherein the template RNA, the nucleic acid molecule encoding the structural polypeptide domain, and/or the nucleic acid molecule encoding the reverse transcriptase polypeptide domain are introduced into the target cell via electroporation (e.g., via nucleofection).
187. The system, template RNA, DNA molecule, or method of any of the preceding embodiments, wherein the template RNA or template DNA does not comprise a primer binding site.
188. The system, template RNA, DNA molecule, or method of any of the preceding embodiments, wherein the template RNA or template DNA does not comprise a 3′ LTR.
189. The system, template RNA, DNA molecule, or method of any of the preceding embodiments, wherein the template RNA or template DNA does not comprise a packaging signal, e.g., in a sequence encoding a structural polypeptide domain and/or in a sequence encoding a reverse transcriptase polypeptide domain.
190. The system, template RNA, DNA molecule, or method of any of the preceding embodiments, wherein the template RNA or template DNA comprises an RNA-transport element (RTE) or a constitutive transport element (CTE).
191. The system, template RNA, DNA molecule, or method of any of the preceding embodiments, wherein an RNA of the system (e.g., template RNA, the RNA encoding the polypeptide of (a), or an RNA expressed from a heterologous object sequence integrated into a target DNA) comprises a microRNA binding site, e.g., in a 3′ UTR.
192. The system, template RNA, DNA molecule, or method of embodiment 191, wherein the microRNA binding site is recognized by a miRNA that is present in a non-target cell type, but that is not present (or is present at a reduced level relative to the non-target cell) in a target cell type.
193. The system, template RNA, DNA molecule, or method of embodiment 191 or 192, wherein the miRNA is miR-142, and/or wherein the non-target cell is a Kupffer cell or a blood cell, e.g., an immune cell.
194. The system, template RNA, DNA molecule, or method of embodiment 191 or 192, wherein the miRNA is miR-182 or miR-183, and/or wherein the non-target cell is a dorsal root ganglion neuron.
195. The system, template RNA, DNA molecule, or method of any of embodiments 191-194, wherein the system comprises a first miRNA binding site that is recognized by a first miRNA (e.g., miR-142) and the system further comprises a second miRNA binding site that is recognized by a second miRNA (e.g., miR-182 or miR-183), wherein the first miRNA binding site and the second miRNA binding site are situated on the same RNA or on different RNAs of the system.
196. The system, fusion protein, or method of any of the preceding embodiments, wherein the system, polypeptide, and/or DNA encoding the same, is formulated as a lipid nanoparticle (LNP).
197. The system, fusion protein, or method of embodiment 196, wherein the lipid nanoparticle (or a formulation comprising a plurality of the lipid nanoparticles) lacks reactive impurities (e.g., aldehydes), or comprises less than a preselected level of reactive impurities (e.g., aldehydes).
198. The system, fusion protein, or method of embodiment 196, wherein the lipid nanoparticle (or a formulation comprising a plurality of the lipid nanoparticles) lacks aldehydes, or comprises less than a preselected level of aldehydes.
199. The system, fusion protein, or method of any of embodiments 196-198, wherein the lipid nanoparticle is comprised in a formulation comprising a plurality of the lipid nanoparticles.
200. The system, fusion protein, or method of embodiment 199, wherein the lipid nanoparticle formulation is produced using one or more lipid reagents comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content.
201. The system, fusion protein, or method of embodiment 200, wherein the lipid nanoparticle formulation is produced using one or more lipid reagents comprising less than 3% total reactive impurity (e.g., aldehyde) content.
202. The system, fusion protein, or method of any of embodiments 199-201, wherein the lipid nanoparticle formulation is produced using one or more lipid reagents comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.
203. The system, fusion protein, or method of embodiment 202, wherein the lipid nanoparticle formulation is produced using one or more lipid reagent comprising less than 0.3% of any single reactive impurity (e.g., aldehyde) species.
204. The system, fusion protein, or method of embodiment 202, wherein the lipid nanoparticle formulation is produced using one or more lipid reagents comprising less than 0.1% of any single reactive impurity (e.g., aldehyde) species.
205. The system, fusion protein, or method of any of embodiments 199-204, wherein the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content.
206. The system, fusion protein, or method of embodiment 205, wherein the lipid nanoparticle formulation comprises less than 3% total reactive impurity (e.g., aldehyde) content.
207. The system, fusion protein, or method of any of embodiments 199-206, wherein the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.
208. The system, fusion protein, or method of embodiment 207, wherein the lipid nanoparticle formulation comprises less than 0.3% of any single reactive impurity (e.g., aldehyde) species.
209. The system, fusion protein, or method of embodiment 207, wherein the lipid nanoparticle formulation comprises less than 0.1% of any single reactive impurity (e.g., aldehyde) species.
210. The system, fusion protein, or method of any of embodiments 196-209, wherein one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content.
211. The system, fusion protein, or method of embodiment 210, wherein one or more, or optionally all, of the lipid reagents used for a lipid nanoparticle as described herein or a formulation thereof comprise less than 3% total reactive impurity (e.g., aldehyde) content.
212. The system, fusion protein, or method of any of embodiments 196-211, wherein one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.
213. The system, fusion protein, or method of embodiment 212, wherein one or more, or optionally all, of the lipid reagents used for a lipid nanoparticle as described herein or a formulation thereof comprise less than 0.3% of any single reactive impurity (e.g., aldehyde) species.
214. The system, fusion protein, or method of embodiment 212, wherein one or more, or optionally all, of the lipid reagents used for a lipid nanoparticle as described herein or a formulation thereof comprise less than 0.1% of any single reactive impurity (e.g., aldehyde) species.
215. The system, fusion protein, or method of any of embodiments 196-214, wherein the total aldehyde content and/or quantity of any single reactive impurity (e.g., aldehyde) species is determined by liquid chromatography (LC), e.g., coupled with tandem mass spectrometry (MS/MS), e.g., as described herein.
216. The system, fusion protein, or method of any of embodiments 196-214, wherein the total aldehyde content and/or quantity of reactive impurity (e.g., aldehyde) species is determined by detecting one or more chemical modifications of a nucleic acid molecule (e.g., as described herein) associated with the presence of reactive impurities (e.g., aldehydes), e.g., in the lipid reagents.
217. The system, fusion protein, or method of any of embodiments 196-214, wherein the total aldehyde content and/or quantity of aldehyde species is determined by detecting one or more chemical modifications of a nucleotide or nucleoside (e.g., a ribonucleotide or ribonucleoside, e.g., comprised in or isolated from a nucleic acid molecule, e.g., as described herein) associated with the presence of reactive impurities (e.g., aldehydes), e.g., in the lipid reagents, e.g., as described herein.
218. The system, fusion protein, or method of embodiment 217, wherein the chemical modifications of a nucleic acid molecule, nucleotide, or nucleoside are detected by determining the presence of one or more modified nucleotides or nucleosides, e.g., using LC-MS/MS analysis, e.g., as described herein.
219. A lipid nanoparticle (LNP) comprising the system, polypeptide (or RNA encoding the same), nucleic acid molecule, or DNA encoding the system or polypeptide, of any preceding embodiment.
220. A system comprising a first lipid nanoparticle comprising the polypeptide (or DNA or RNA encoding the same) of a Gene Writing system (e.g., as described herein); and
   a second lipid nanoparticle comprising a nucleic acid molecule of a Gene Writing System (e.g., as described herein).
221. The system, fusion protein, or method of any preceding embodiment, wherein the system, nucleic acid molecule, polypeptide, and/or DNA encoding the same, is formulated as a lipid nanoparticle (LNP).
222. The LNP of embodiment 221, comprising a cationic lipid.
223. The LNP of embodiment 221 or 222, wherein the cationic lipid has a structure according to:

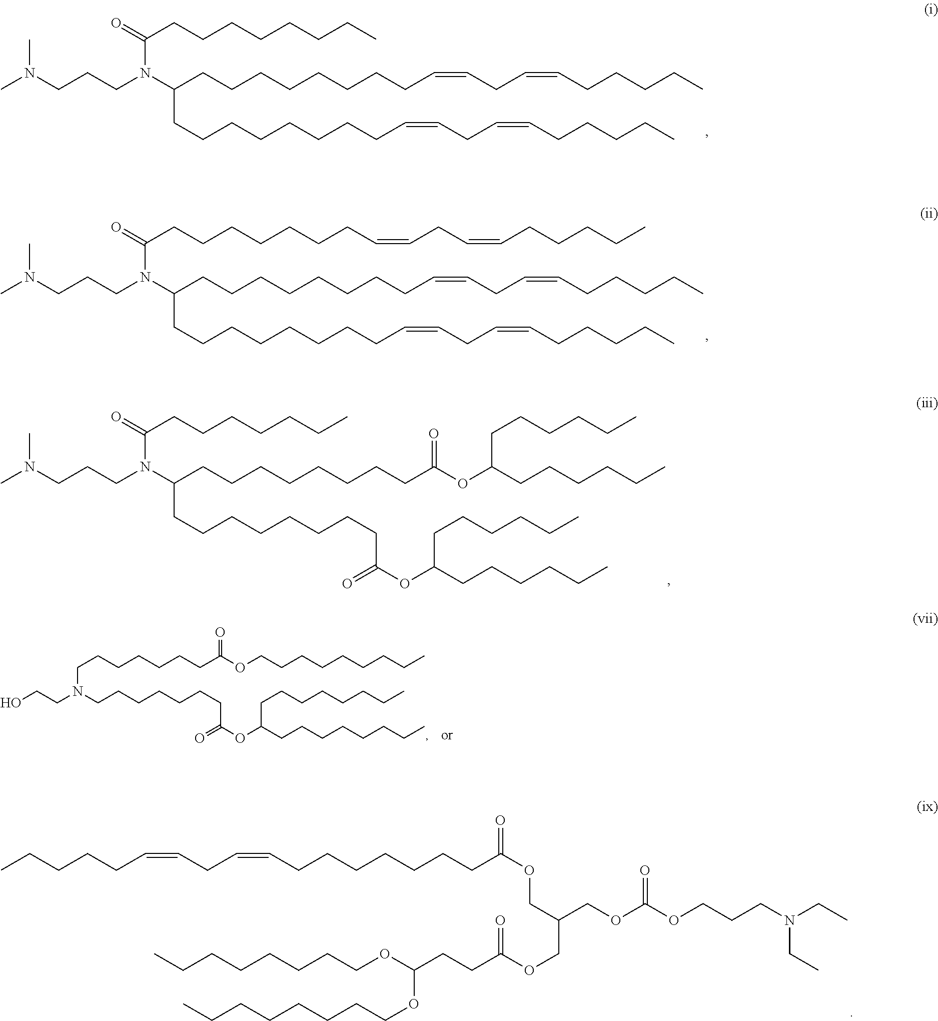

224. The LNP of any of embodiments 221-223, further comprising one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.
225. The system, fusion protein, or method of any of the preceding embodiments, wherein the system comprises one or more circular RNA molecules (circRNAs).
226. The system, fusion protein, or method of embodiment 225, wherein the circRNA encodes the Gene Writer polypeptide.
227. The system, fusion protein, or method of any of embodiments 225-226, wherein circRNA is delivered to a host cell.
228. The system, fusion protein, or method of any of the preceding embodiments, wherein the circRNA is capable of being linearized, e.g., in a host cell, e.g., in the nucleus of the host cell.
229. The system, fusion protein, or method of any of the preceding embodiments, wherein the circRNA comprises a cleavage site.
230. The system, fusion protein, or method of embodiment 229, wherein the circRNA further comprises a second cleavage site.
231. The system, fusion protein, or method of embodiment 229 or 230, wherein the cleavage site can be cleaved by a ribozyme, e.g., a ribozyme comprised in the circRNA (e.g., by autocleavage).
232. The system, fusion protein, or method of any of the preceding embodiments, wherein the circRNA comprises a ribozyme sequence.
233. The system, fusion protein, or method of embodiment 232, wherein the ribozyme sequence is capable of autocleavage, e.g., in a host cell, e.g., in the nucleus of the host cell.
234. The system, fusion protein, or method of any of embodiments 232-233, wherein the ribozyme is an inducible ribozyme.
235. The system, fusion protein, or method of any of embodiments 232-234 wherein the ribozyme is a protein-responsive ribozyme, e.g., a ribozyme responsive to a nuclear protein, e.g., a genome-interacting protein, e.g., an epigenetic modifier, e.g., EZH2.
236. The system, fusion protein, or method of any of embodiments 232-235, wherein the ribozyme is a nucleic acid-responsive ribozyme.
237. The system, fusion protein, or method of embodiment 236, wherein the catalytic activity (e.g., autocatalytic activity) of the ribozyme is activated in the presence of a target nucleic acid molecule (e.g., an RNA molecule, e.g., an mRNA, miRNA, ncRNA, lncRNA, tRNA, snRNA, or mtRNA).
238. The system, fusion protein, or method of any of embodiments 232-235, wherein the ribozyme is responsive to a target protein (e.g., an MS2 coat protein).
239. The system, fusion protein, or method of embodiment 238, wherein the target protein localized to the cytoplasm or localized to the nucleus (e.g., an epigenetic modifier or a transcription factor).
240. The system, fusion protein, or method of any of embodiments 232-236, wherein the ribozyme comprises the ribozyme sequence of a B2 or ALU retrotransposon, or a nucleic acid sequence having at least 85%, 90%, 95%, 96%, 97%, 98%, or 99% sequence identity thereto.
241. The system, fusion protein, or method of any of embodiments 232-236, wherein the ribozyme comprises the sequence of a tobacco ringspot virus hammerhead ribozyme, or a nucleic acid sequence having at least 85%, 90%, 95%, 96%, 97%, 98%, or 99% sequence identity thereto.
242. The system, fusion protein, or method of any of embodiments 232-236, wherein the ribozyme comprises the sequence of a hepatitis delta virus (HDV) ribozyme, or a nucleic acid sequence having at least 85%, 90%, 95%, 96%, 97%, 98%, or 99% sequence identity thereto.
243. The system, fusion protein, or method of any of embodiments 232-242, wherein the ribozyme is activated by a moiety expressed in a target cell or target tissue.
244. The system, fusion protein, or method of any of embodiments 232-243, wherein the ribozyme is activated by a moiety expressed in a target subcellular compartment (e.g., a nucleus, nucleolus, cytoplasm, or mitochondria).
245. The system, fusion protein, or method of any of the preceding embodiments, wherein the ribozyme is comprised in a circular RNA or a linear RNA.
246. A system comprising a first circular RNA encoding the polypeptide of a Gene Writing system; and a second circular RNA comprising the template RNA of a Gene Writing system.
247. The system of any of the preceding embodiments, wherein the template RNA, e.g., the 5′ UTR, comprises a ribozyme which cleaves the template RNA (e.g., in the 5′ UTR).
248. The system of any of the preceding embodiments, wherein the template RNA comprises a ribozyme that is heterologous to (a)(i), (a)(ii), (b)(i), or a combination thereof.
249. The system of any of the preceding embodiments, wherein the heterologous ribozyme is capable of cleaving RNA comprising the ribozyme, e.g., 5′ of the ribozyme, 3′ of the ribozyme, or within the ribozyme.
250. A method of making a system for modifying DNA (e.g., as described herein), the method comprising:
   (a) providing a template nucleic acid (e.g., a template RNA or DNA) comprising a heterologous homology sequence having at least 85%, 90%, 95%, 96%, 97%, 98%, 99%, or 100% homology to a sequence comprised in a target DNA molecule, and/or
   (b) providing a polypeptide of the system (e.g., comprising a DNA-binding domain (DBD) and/or an endonuclease domain) comprising a heterologous targeting domain that binds specifically to a sequence comprised in the target DNA molecule.
251. The method of embodiment 250, wherein:
   (a) comprises introducing into the template nucleic acid (e.g., a template RNA or DNA) a heterologous homology sequence having at least 85%, 90%, 95%, 96%, 97%, 98%, 99%, or 100% homology to the sequence comprised in a target DNA molecule, and/or
   (b) comprises introducing into the polypeptide of the system (e.g., comprising a DNA-binding domain (DBD) and/or an endonuclease domain) the heterologous targeting domain that binds specifically to a sequence comprised in the target DNA molecule.
252. The method of embodiment 251, wherein the introducing of (a) comprises inserting the homology sequence into the template nucleic acid.
253. The method of embodiment 251, wherein the introducing of (a) comprises replacing a segment of the template nucleic acid with the homology sequence.
254. The method of embodiment 251, wherein the introducing of (a) comprises mutating one or more nucleotides (e.g., at least 2, 3, 4, 5, 10, 15, 20, 25, 30, 35, 40, 50, 60, 70, 80, 90, or 100 nucleotides) of the template nucleic acid, thereby producing a segment of the template nucleic acid having the sequence of the homology sequence.
255. The method of embodiment 251, wherein the introducing of (b) comprises inserting the amino acid sequence of the targeting domain into the amino acid sequence of the polypeptide.
256. The method of embodiment 255, wherein the introducing of (b) comprises inserting a nucleic acid sequence encoding the targeting domain into a coding sequence of the polypeptide comprised in a nucleic acid molecule.
257. The method of embodiment 255, wherein the introducing of (b) comprises replacing at least a portion of the polypeptide with the targeting domain.
258. The method of embodiment 251, wherein the introducing of (a) comprises mutating one or more amino acids (e.g., at least 1, 2, 3, 4, 5, 6, 7, 8, 9, 10, 15, 20, 25, 30, 40, 50, 60, 70, 80, 90, 100, 150, 200, 250, 300, 400, 500, or more amino acids) of the polypeptide.
259. A method for modifying a target site in genomic DNA in a cell, the method comprising contacting the cell with:
   (a) a polypeptide or a nucleic acid encoding the polypeptide, wherein the polypeptide comprises (i) a reverse transcriptase (RT) domain, (ii) a DNA-binding domain (DBD); and (iii) an endonuclease domain, e.g., a nickase domain; and
   (b) a template RNA (or DNA encoding the template RNA) comprising (e.g., from 5′ to 3′) (i) optionally a sequence that binds the target site (e.g., a second strand of a site in a target genome), (ii) optionally a sequence that binds the polypeptide, (iii) a heterologous object sequence, and (iv) a 3′ target homology domain,
   wherein:
      (i) the polypeptide comprises a heterologous targeting domain (e.g., in the DBD or the endonuclease domain) that binds specifically to a sequence comprised in or adjacent to the target site of the genomic DNA; and/or
      (ii) the template RNA comprises a heterologous homology sequence having at least 85%, 90%, 95%, 96%, 97%, 98%, 99%, or 100% homology to a sequence comprised in or adjacent to the target site of the genomic DNA;
   thereby modifying the target site in genomic DNA in a cell.
260. A system for modifying DNA comprising:
   (a) a polypeptide or a nucleic acid encoding the polypeptide, wherein the polypeptide comprises (i) a reverse transcriptase (RT) domain, (ii) a DNA-binding domain (DBD); and (iii) an endonuclease domain, e.g., a nickase domain; and
   (b) a template RNA (or DNA encoding the template RNA) comprising (e.g., from 5′ to 3′) (i) optionally a sequence that binds a target site (e.g., a second strand of a site in a target genome), (ii) optionally a sequence that binds the polypeptide, (iii) a heterologous object sequence, and (iv) a 3′ target homology domain;
   wherein:
      (i) the polypeptide comprises a heterologous targeting domain (e.g., in the DBD or the endonuclease domain) that binds specifically to a sequence comprised in the target site; and/or
      (ii) the template RNA comprises a heterologous homology sequence having at least 85%, 90%, 95%, 96%, 97%, 98%, 99%, or 100% homology to a sequence comprised in a target site.
261. A template RNA (or DNA encoding the template RNA) comprising a targeting domain (e.g., a heterologous targeting domain) that binds specifically to a sequence comprised in the target DNA molecule (e.g., a genomic DNA), a sequence that specifically binds an RT domain of a polypeptide, and a heterologous object sequence.
262. A polypeptide or a nucleic acid encoding the polypeptide, wherein the polypeptide comprises (i) a reverse transcriptase (RT) domain, (ii) a DNA-binding domain (DBD); and (iii) an endonuclease domain; wherein the DBD and/or the endonuclease domain comprise a heterologous targeting domain that binds specifically to a sequence comprised in a target DNA molecule (e.g., a genomic DNA).
263. The system, fusion protein, or method of any of the preceding embodiments, wherein the polypeptide comprises a heterologous targeting domain that binds specifically to a sequence comprised in the target DNA molecule (e.g., a genomic DNA).
264. The system, fusion protein, or method of embodiment 263, wherein the heterologous target domain binds to a different nucleic acid sequence than the unmodified polypeptide.
265. The system, fusion protein, or method of embodiment 263 or 264, wherein the polypeptide does not comprise a functional endogenous targeting domain (e.g., wherein the polypeptide does not comprise an endogenous targeting domain).
266. The system, fusion protein, or method of any of embodiments 263-265, wherein the heterologous targeting domain comprises a zinc finger (e.g., a zinc finger that binds specifically to the sequence comprised in the target DNA molecule).
267. The system, fusion protein, or method of any of embodiments 263-266, wherein the heterologous targeting domain comprises a Cas domain (e.g., a Cas9 domain, or a mutant or variant thereof, e.g., a Cas9 domain that binds specifically to the sequence comprised in the target DNA molecule).
268. The system, fusion protein, or method of embodiment 267, wherein the Cas domain is associated with a guide RNA (gRNA).
269. The system, fusion protein, or method of any of embodiments 623-268, wherein the heterologous targeting domain comprises an endonuclease domain (e.g., a heterologous endonuclease domain).
270. The system, fusion protein, or method of embodiment 269, wherein the endonuclease domain comprises a Cas domain (e.g., a Cas9 or a mutant or variant thereof).
271. The system, fusion protein, or method of embodiment 270, wherein the Cas domain is associated with a guide RNA (gRNA).
272. The system, fusion protein, or method of embodiment 269, wherein the endonuclease domain comprises a Fok1 domain.
273. The system, fusion protein, or method of any of the preceding embodiments, wherein the template nucleic acid molecule comprises at least one (e.g., one or two) heterologous homology sequence having at least 85%, 90%, 95%, 96%, 97%, 98%, 99%, or 100% homology to a sequence comprised in a target DNA molecule (e.g., a genomic DNA).
274. The system, fusion protein, or method of embodiment 273, wherein one of the at least one heterologous homology sequences is positioned at or within about 1, 2, 3, 4, 5, 6, 7, 8, 9, 10, 15, 20, 25, 30, 35, 40, 45, 50, 60, 70, 80, 90, or 100 nucleotides of the 5′ end of the template nucleic acid molecule.
275. The system, fusion protein, or method of embodiment 273 or 274, wherein one of the at least one heterologous homology sequences is positioned at or within about 1, 2, 3, 4, 5, 6, 7, 8, 9, 10, 15, 20, 25, 30, 35, 40, 45, 50, 60, 70, 80, 90, or 100 nucleotides of the 3′ end of the template nucleic acid molecule.
276. The system, fusion protein, or method of embodiment 275, wherein the heterologous homology sequence binds within 1, 2, 3, 4, 5, 6, 7, 8, 9, or 10 nucleotides of a nick site (e.g., produced by a nickase, e.g., an endonuclease domain, e.g., as described herein) in the target DNA molecule.
277. The system, fusion protein, or method of embodiment 273, wherein the heterologous homology sequence has less than 50%, 40%, 30%, 20%, 10%, 5%, 4%, 3%, 2%, or 1% sequence identity with a nucleic acid sequence complementary to an endogenous homology sequence of an unmodified form of the template RNA.
278. The system, fusion protein, or method of embodiment 277, wherein the heterologous homology sequence has having at least 85%, 90%, 95%, 96%, 97%, 98%, 99%, or 100% homology to a sequence of the target DNA molecule that is different the sequence bound by an endogenous homology sequence (e.g., replaced by the heterologous homology sequence).
279. The system, fusion protein, or method of embodiment 273 or 277, wherein the heterologous homology sequence comprises a sequence (e.g., at its 3′ end) having at least 85%, 90%, 95%, 96%, 97%, 98%, 99%, or 100% homology to a sequence positioned 5′ to a nick site of the target DNA molecule (e.g., a site nicked by a nickase, e.g., an endonuclease domain as described herein).
280. The system, fusion protein, or method of any of embodiments 273-279, wherein the heterologous homology sequence comprises a sequence (e.g., at its 5′ end) suitable for priming target-primed reverse transcription (TPRT) initiation.
281. The system, fusion protein, or method of any of embodiments 273-280, wherein the heterologous homology sequence has at least 85%, 90%, 95%, 96%, 97%, 98%, 99%, or 100% homology to a sequence positioned within about 1, 2, 3, 4, 5, 6, 7, 8, 9, 10, 15, 20, 25, 30, 35, 40, 45, 50, 60, 70, 80, 90, or 100 nucleotides of (e.g., 3′ relative to) a target insertion site, e.g., for a heterologous object sequence (e.g., as described herein), in the target DNA molecule.
282. The system, fusion protein, or method of any of embodiments 273-281, wherein the template nucleic acid molecule comprises a guide RNA (gRNA), e.g., as described herein.
283. The system, fusion protein, or method of embodiment 282, wherein the template nucleic acid molecule comprises a gRNA spacer sequence (e.g., at or within 1, 2, 3, 4, 5, 10, 15, 20, 25, 30, 40, 50, 60, 70, 80, 90, or 100 nucleotides of its 5′ end).
284. A template RNA (or DNA encoding the template RNA) comprising (e.g., from 5′ to 3′) (i) a sequence that binds a target site (e.g., a second strand of a site in a target genome), (ii) a sequence that specifically binds an RT domain of a polypeptide, (iii) a heterologous object sequence, and (iv) a 3′ target homology domain.
285. The template RNA of embodiment 284, further comprising (v) a sequence that binds an endonuclease and/or a DNA-binding domain of a polypeptide (e.g., the same polypeptide comprising the RT domain).
286. The template RNA of either of embodiments 284 or 285, wherein the RT domain comprises a sequence selected of Table 1 or 3 or a sequence of a reverse transcriptase domain of Table 2 or a sequence that has at least 70%, 75%, 80%, 85%, 90%, 95%, 96%, 97%, 98%, or 99% identity thereto.
287. The template RNA of any of embodiments 284-286, wherein the RT domain comprises a sequence selected of Table 1 or 3 or a sequence of a reverse transcriptase domain of Table 2, wherein the RT domain further comprises a number of substitutions relative to the natural sequence, e.g., at least 1, 2, 3, 4, 5, 10, 20, 30, 40, 50, 60, 70, 80, 90, or 100 substitutions.
288. The template RNA of embodiments 284-287, wherein the sequence of (ii) specifically binds the RT domain.
289. The template RNA of any of embodiments 284-288, wherein the sequence that specifically binds the RT domain is a sequence, e.g., a UTR sequence, of Table 1 or from a domain of Table 2, or a sequence having at least 70, 75, 80, 85, 90, 95, or 99% identity thereto.
290. A template RNA (or DNA encoding the template RNA) comprising from 5′ to 3′: (ii) a sequence that binds an endonuclease and/or a DNA-binding domain of a polypeptide, (i) a sequence that binds a target site (e.g., a second strand of a site in a target genome), (iii) a heterologous object sequence, and (iv) a 3′ target homology domain.
291. A template RNA (or DNA encoding the template RNA) comprising from 5′ to 3′: (iii) a heterologous object sequence, (iv) a 3′ target homology domain, (i) a sequence that binds a target site (e.g., a second strand of a site in a target genome), and (ii) a sequence that binds an endonuclease and/or a DNA-binding domain of a polypeptide.
292. The system, template RNA, DNA molecule, or method of any of the preceding embodiments, wherein the template RNA comprises at least 2, 3, or 4 miRNA binding sites, e.g., wherein the miRNA binding sites are recognized by the same or different miRNAs.
293. The system, template RNA, DNA molecule, or method of any of the preceding embodiments, wherein the RNA encoding the polypeptide of (a) comprises at least 2, 3, or 4 miRNA binding sites, e.g., wherein the miRNA binding sites are recognized by the same or different miRNAs.
294. The system, template RNA, DNA molecule, or method of any of the preceding embodiments, wherein the RNA expressed from a heterologous object sequence integrated into a target DNA comprises at least 2, 3, or 4 miRNA binding sites, e.g., wherein the miRNA binding sites are recognized by the same or different miRNAs.
295. The system, template RNA, DNA molecule, or method of any of the preceding embodiments, wherein the system comprises one or more elements comprising a sequence as set out in Table S1, or a sequence having at least 70%, 75%, 80%, 85%, 90%, 95%, 96%, 97%, 98%, or 99% identity thereto.
296. The system, template RNA, DNA molecule, or method of any of the preceding embodiments, wherein the system comprises one or more elements comprising a sequence as set out in Table S2, or a sequence having at least 70%, 75%, 80%, 85%, 90%, 95%, 96%, 97%, 98%, or 99% identity thereto.
297. The system, template RNA, DNA molecule, or method of any of the preceding embodiments, wherein the system comprises one or more elements comprising a sequence as set out in Table S3, or a sequence having at least 70%, 75%, 80%, 85%, 90%, 95%, 96%, 97%, 98%, or 99% identity thereto.
298. The system, template RNA, DNA molecule, or method of any of the preceding embodiments, wherein the system comprises one or more elements comprising a sequence as set out in Table S4, or a sequence having at least 70%, 75%, 80%, 85%, 90%, 95%, 96%, 97%, 98%, or 99% identity thereto.
299. The system, template RNA, DNA molecule, or method of any of the preceding embodiments, wherein the system comprises one or more elements comprising a sequence as set out in Table S5, or a sequence having at least 70%, 75%, 80%, 85%, 90%, 95%, 96%, 97%, 98%, or 99% identity thereto.
300. The system, template RNA, DNA molecule, or method of any of the preceding embodiments, wherein the template RNA, the nucleic acid molecule encoding the structural polypeptide domain, and the nucleic acid molecule encoding the reverse transcriptase polypeptide domain are comprised in the same nucleic acid molecule.
301. The system, template RNA, DNA molecule, or method of any of the preceding embodiments, wherein the template RNA and the nucleic acid molecules encoding the structural polypeptide domain and/or the reverse transcriptase polypeptide domain are comprised in different nucleic acid molecules.

### Definitions

About, approximately: “About” or “approximately” as the terms are used herein applied to one or more values of interest, refer to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Domain: The term “domain” as used herein refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, a nuclear localization sequence, a recombinase domain, a retroviral (e.g., endogenous retroviral) structural polypeptide domain, a retroviral (e.g., endogenous retroviral) reverse transcriptase polypeptide domain, a retrotransposon structural polypeptide domain, a retrotransposon reverse transcriptase polypeptide domain, a DNA recognition domain (e.g., that binds to or is capable of binding to a recognition site, e.g. as described herein), a recombinase N-terminal domain (also called a catalytic domain), a C-terminal zinc ribbon domain. In some embodiments the zinc ribbon domain further comprises a coiled-coiled motif. In some embodiments, the recombinase domain and the zinc ribbon domain are collectively referred to as the C-terminal domain. In some embodiments the N-terminal domain is linked to the C-terminal domain by an aE linker or helix. In some embodiments the N-terminal domain is between 50 and 250 amino acids, or 100-200 amino acids, or 130-170 amino acids, e.g., about 150 amino acids. In some embodiments the C-terminal domain is 200-800 amino acids, or 300-500 amino acids. In some embodiments the recombinase domain is between 50 and 150 amino acids. In some embodiments the zinc ribbon domain is between 30 and 100 amino acids; an example of a domain of a nucleic acid is a regulatory domain, such as a transcription factor binding domain, a recognition sequence, an arm of a recognition sequence (e.g. a 5′ or 3′ arm), a core sequence, or an object sequence (e.g., a heterologous object sequence).

Exogenous: As used herein, the term exogenous, when used with reference to a biomolecule (such as a nucleic acid sequence or polypeptide) means that the biomolecule was introduced into a host genome, cell or organism by the hand of man. For example, a nucleic acid that is as added into an existing genome, cell, tissue or subject using recombinant DNA techniques or other methods is exogenous to the existing nucleic acid sequence, cell, tissue or subject.

Genomic safe harbor site (GSH site): A genomic safe harbor site is a site in a host genome that is able to accommodate the integration of new genetic material, e.g., such that the inserted genetic element does not cause significant alterations of the host genome posing a risk to the host cell or organism. A GSH site generally meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconservered element; (vi) has low transcriptional activity (i.e. no mRNA+/−25 kb); (vii) is not in copy number variable region; (viii) is in open chromatin; and/or (ix) is unique, with 1 copy in the human genome. Examples of GSH sites in the human genome that meet some or all of these criteria include (i) the adeno-associated virus site 1 (AAVS1), a naturally occurring site of integration of AAV virus on chromosome 19; (ii) the chemokine (C-C motif) receptor 5 (CCR5) gene, a chemokine receptor gene known as an HIV-1 coreceptor; (iii) the human ortholog of the mouse Rosa26 locus; (iv) the rDNA locus. Additional GSH sites are known and described, e.g., in Pellenz et al. epub Aug. 20, 2018 (https://doi.org/10.1101/396390).

Heterologous: The term heterologous, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector). In some embodiments, a domain is heterologous relative to another domain, if the first domain is not naturally comprised in the same polypeptide as the other domain (e.g., a fusion between two domains of different proteins from the same organism).

Long Terminal Repeat: The term “long terminal repeat” (LTR), as used herein, refers to a nucleic acid sequence, which in a wild-type context are found in pairs (which may be identical or have sequence similarity) that flank a retrovirus or an LTR retrotransposon. The term “LTR” also encompasses variants and fragments of a wild-type LTR which are functional for integration of a region of the nucleic acid molecule comprising the LTR into a target DNA molecule in the presence of factors from the retrovirus or LTR retrotransposon. An LTR is typically located at or near one end (e.g., the 5′ end or the 3′ end) of a template DNA or RNA, e.g., as described herein. In some instances, an LTR participates in integration of a heterologous object sequence comprised in the template DNA or RNA into a target DNA molecule (e.g., a genomic DNA). In some instances, the LTR, or a fragment thereof, is integrated into the target DNA molecule. In some instances, the LTR is not integrated into the target DNA molecule. In some instances, a first LTR of a template DNA or RNA (e.g., as described herein) has at least 50%, 60%, 70%, 75%, 80%, 85%, 90%, 95%, 96%, 97%, 98%, 99%, or 100% sequence identity to a second LTR sequence of the template DNA or RNA. In some instances, an LTR of a system or composition described herein has at least 75%, 80%, 85%, 90%, 95%, 96%, 97%, 98%, or 99% sequence identity to an LTR sequence of a naturally occurring retrovirus (e.g., endogenous retrovirus) or LTR retrotransposon. In some instances, an LTR of a system or composition described herein has at least one modification (e.g., an addition, substitution, or deletion) relative to an LTR sequence of a naturally occurring retrovirus (e.g., endogenous retrovirus) or LTR retrotransposon. In some embodiments, an LTR has promoter and/or enhancer activity.

Mutation or Mutated: The term “mutated” when applied to nucleic acid sequences means that nucleotides in a nucleic acid sequence may be inserted, deleted or changed compared to a reference (e.g., native) nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art. In some embodiments a mutation occurs naturally. In some embodiments a desired mutation can be produced by any suitable method.

Nucleic acid molecule: Nucleic acid molecule refers to both RNA and DNA molecules including, without limitation, cDNA, genomic DNA and mRNA, and also includes synthetic nucleic acid molecules, such as those that are chemically synthesized or recombinantly produced, such as RNA templates, as described herein. The nucleic acid molecule can be double-stranded or single-stranded, circular or linear. If single-stranded, the nucleic acid molecule can be the sense strand or the antisense strand. Unless otherwise indicated, and as an example for all sequences described herein under the general format “SEQ. ID NO:,” “nucleic acid comprising SEQ. ID NO:1” refers to a nucleic acid, at least a portion which has either (i) the sequence of SEQ. ID NO:1, or (ii) a sequence complementary to SEQ. ID NO:1. The choice between the two is dictated by the context in which SEQ. ID NO:1 is used. For instance, if the nucleic acid is used as a probe, the choice between the two is dictated by the requirement that the probe be complementary to the desired target. Nucleic acid sequences of the present disclosure may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule. Other modifications can include, for example, analogs in which the ribose ring contains a bridging moiety or other structure such as modifications found in “locked” nucleic acids.

Gene expression unit: a gene expression unit is a nucleic acid sequence comprising at least one regulatory nucleic acid sequence operably linked to at least one effector sequence. A first nucleic acid sequence is operably linked with a second nucleic acid sequence when the first nucleic acid sequence is placed in a functional relationship with the second nucleic acid sequence. For instance, a promoter or enhancer is operably linked to a coding sequence if the promoter or enhancer affects the transcription or expression of the coding sequence. Operably linked DNA sequences may be contiguous or non-contiguous. Where necessary to join two protein-coding regions, operably linked sequences may be in the same reading frame.

Host: The terms host genome or host cell, as used herein, refer to a cell and/or its genome into which protein and/or genetic material has been introduced. It should be understood that such terms are intended to refer not only to the particular subject cell and/or genome, but to the progeny of such a cell and/or the genome of the progeny of such a cell. Because certain modifications may occur in succeeding generations due to either mutation or environmental influences, such progeny may not, in fact, be identical to the parent cell, but are still included within the scope of the term “host cell” as used herein. A host genome or host cell may be an isolated cell or cell line grown in culture, or genomic material isolated from such a cell or cell line, or may be a host cell or host genome which composing living tissue or an organism. In some instances, a host cell may be an animal cell or a plant cell, e.g., as described herein. In certain instances, a host cell may be a bovine cell, horse cell, pig cell, goat cell, sheep cell, chicken cell, or turkey cell. In certain instances, a host cell may be a corn cell, soy cell, wheat cell, or rice cell.

Introducing: As used herein, the term “introducing”, in the context of introducing an agent into a call, refers to causing the agent to be comprised by the cell. For example, the cell may be contacted with the agent in a way that allows the agent to pass through the cell membrane to enter the cell. Alternatively, the agent can be introduced into the cell by causing the cell to produce the agent. For instance, an agent that is a polypeptide can be introduced into the cell by contacting the cell with a nucleic acid encoding the polypeptide, under conditions that the nucleic acid enters the cell and is translated to produce the polypeptide.

Contacting: As used herein, the term “contacting”, in the context of contacting a cell with an agent, comprises placing the agent at a location that allows the agent to come into physical contact with the cell. Physical contact with the cell includes, e.g., binding to the cell surface or being internalized into the cell. In some embodiments, e.g., ex vivo, contacting a cell with an agent comprises introducing the agent into media, wherein the media is in contact with the cell. In some embodiments, e.g., in vivo, contacting a cell with an agent comprises administering the agent to a subject comprising the cell, under conditions that allow the agent to come into physical contact with the cell.

Object sequence: As used herein, the term object sequence refers to a nucleic acid segment that can be desirably inserted into a target nucleic acid molecule, e.g., by a recombinase polypeptide, e.g., as described herein. In some embodiments, a template RNA or template DNA comprises a DNA recognition sequence and an object sequence that is heterologous to the DNA recognition sequence and/or the remainder of the template RNA or template DNA, generally referred to herein as a “heterologous object sequence.” An object sequence may, in some instances, be heterologous relative to the nucleic acid molecule into which it is inserted (e.g., a target DNA molecule, e.g., as described herein). In some instances, an object sequence comprises a nucleic acid sequence encoding a gene (e.g., a eukaryotic gene, e.g., a mammalian gene, e.g., a human gene) or other cargo of interest (e.g., a sequence encoding a functional RNA, e.g., an siRNA or miRNA), e.g., as described herein. In certain instances, the gene encodes a polypeptide (e.g., a blood factor or enzyme). In some instances, an object sequence comprises one or more of a nucleic acid sequence encoding a selectable marker (e.g., an auxotrophic marker or an antibiotic marker), and/or a nucleic acid control element (e.g., a promoter, enhancer, silencer, or insulator).

Pseudoknot: A “pseudoknot sequence” sequence, as used herein, refers to a nucleic acid (e.g., RNA) having a sequence with suitable self-complementarity to form a pseudoknot structure, e.g., having: a first segment, a second segment between the first segment and a third segment, wherein the third segment is complementary to the first segment, and a fourth segment, wherein the fourth segment is complementary to the second segment. The pseudoknot may optionally have additional secondary structure, e.g., a stem loop disposed in the second segment, a stem-loop disposed between the second segment and third segment, sequence before the first segment, or sequence after the fourth segment. The pseudoknot may have additional sequence between the first and second segments, between the second and third segments, or between the third and fourth segments. In some embodiments, the segments are arranged, from 5′ to 3′: first, second, third, and fourth. In some embodiments, the first and third segments comprise five base pairs of perfect complementarity. In some embodiments, the second and fourth segments comprise 10 base pairs, optionally with one or more (e.g., two) bulges. In some embodiments, the second segment comprises one or more unpaired nucleotides, e.g., forming a loop. In some embodiments, the third segment comprises one or more unpaired nucleotides, e.g., forming a loop.

Stem-loop sequence: As used herein, a “stem-loop sequence” refers to a nucleic acid sequence (e.g., RNA sequence) with sufficient self-complementarity to form a stem-loop, e.g., having a stem comprising at least two (e.g., 3, 4, 5, 6, 7, 8, 9, or 10) base pairs, and a loop with at least three (e.g., four) base pairs. The stem may comprise mismatches or bulges.

Structural polypeptide domain: As used herein, the term “structural polypeptide domain” refers to a polypeptide domain that can form part of a proteinaceous exterior (e.g., a viral capsid) encapsulating a viral nucleic acid (e.g., a template RNA, e.g., as described herein). Retroviral env is not a structural polypeptide domain, as the term is used herein. In some instances, a structural polypeptide domain is encoded by a viral gene (e.g., a retroviral gag gene). In some instances, a structural polypeptide domain comprises a capsid protein (e.g., a CA protein and/or an NC protein, e.g., encoded by a retroviral gag gene), or a functional fragment thereof. In some instances, a structural polypeptide domain comprises a matrix protein (e.g., a MA protein, e.g., encoded by a retroviral gag gene), or a functional fragment thereof. In some instances, a structural polypeptide domain comprises a domain encoded by a retroviral gag (e.g., an endogenous retroviral gag). In some embodiments, a structural polypeptide domain comprises one or more mutations (e.g., point mutations, additions, substitutions, or deletions) relative to the amino acid sequence of a corresponding wild-type protein (e.g., a wild-type retroviral gag, CA, NC, or MA protein). In some embodiments, a structural polypeptide domain is part of a polyprotein or a fusion protein. In some embodiments, a structural polypeptide domain is not part of a polyprotein or a fusion protein.

Reverse transcriptase domain: As used herein, the term “reverse transcriptase domain” refers to a polypeptide domain capable of producing complementary DNA from a template RNA (e.g., as described herein). In some instances, a reverse transcriptase domain comprises a viral (e.g., retroviral, e.g., endogenous retroviral) reverse transcriptase, or a functional fragment thereof. In some instances, a reverse transcriptase domain produces complementary DNA from a template RNA via a primer (e.g., a tRNA primer, e.g., a lysyl tRNA primer). In some instances, a reverse transcriptase domain produces a double stranded template DNA (e.g., as described herein) from the template RNA. In some instances, a reverse transcriptase domain is encoded by a viral (e.g., retroviral, e.g., endogenous retroviral) pol gene. In some instances, a reverse transcriptase domain is encoded by a pol gene that also encodes a viral (e.g., retroviral, e.g., endogenous retroviral) integrase (IN). In some instances, a reverse transcriptase domain is encoded by a pol gene that also encodes a viral (e.g., retroviral, e.g., endogenous retroviral) protease (PR) and/or dTUPase (DU). In some embodiments, a reverse transcriptase polypeptide domain comprises one or more mutations (e.g., point mutations, additions, substitutions, or deletions) relative to the amino acid sequence of a corresponding wild-type protein (e.g., a wild-type retroviral pol, IN, PR, or DU protein). In some embodiments, a reverse transcriptase domain is part of a polyprotein or a fusion protein. In some embodiments, a reverse transcriptase domain is not part of a polyprotein or a fusion protein. In some embodiments, the reverse transcriptase domain comprises RNaseH activity. In some embodiments, a functional reverse transcriptase comprises a single protein subunit, e.g., is monomeric. In some embodiments, a functional reverse transcriptase comprises at least two subunits, e.g., is dimeric. In some embodiments, the reverse transcriptase domain is less active (or inactive) in monomeric form compared to in dimeric form. In some embodiments, a dimeric reverse transcriptase comprises two identical subunits. In some embodiments, a dimeric reverse transcriptase comprises different subunits, e.g., a p51 and a p66 subunit. In some embodiments, a reverse transcriptase comprises at least three subunits, e.g., two p51 subunits and at least one p15 subunit. In some embodiments, a reverse transcriptase comprises an RNase H domain. In some embodiments, a reverse transcriptase comprises an inactivated RNase H domain. In some embodiments, a reverse transcriptase does not comprise an RNase H domain.

LTR retrotransposon: As used herein, the term “LTR retrotransposon” in the context of a domain (e.g., LTR retrotransposon structural polypeptide domain or LTR retrotransposon reverse transcriptase polypeptide domain) refers to a polypeptide domain having sequence similarity to a corresponding domain from a wild-type LTR retrotransposon, and at least one biological function (e.g., capsid formation or reverse transcription) in common with the corresponding domain. A wild-type LTR retrotransposon does not comprise an env gene. In some embodiments, an LTR retrotransposon may comprise a retrovirus (eg an endogenous retrovirus) engineered to lacka functional env gene.

Retroviral: As used herein, the term “retroviral” in the context of a domain (e.g., retroviral structural polypeptide domain or retroviral reverse transcriptase polypeptide domain) refers to a polypeptide domain having sequence similarity to a corresponding domain from a wild-type retrovirus (e.g., endogenous retrovirus) and at least one biological function (e.g., capsid formation or reverse transcription) in common with the corresponding domain. A wild-type retrovirus comprises an env gene.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 schematically shows an exemplary LTR or endogenous retrovirus (ERV) engineered for integrating a gene into a genome and delivered in the form of episomal DNA.

FIG. 2 schematically shows an exemplary LTR or ERV engineered for integrating a gene into a genome and delivered in the form of RNA.

FIG. 3 schematically shows an exemplary LTR or ERV engineered for introducing a gene episomally and delivered in the form of RNA.

FIG. 4 schematically shows an exemplary LTR or ERV engineered for integrating an intron-bearing gene into a genome and delivered in the form of RNA.

FIG. 5 schematically shows exemplary strategies for modifying an ERV or a retrovirus to be an LTR retrotransposon.

FIG. 6 schematically shows the design of an exemplary template.

FIGS. 7A and 7B describe luciferase activity assay for primary cells. LNPs formulated as according to Example 2 were analyzed for delivery of cargo to primary human (A) and mouse (B) hepatocytes, as according to Example 3. The luciferase assay revealed dose-responsive luciferase activity from cell lysates, indicating successful delivery of RNA to the cells and expression of Firefly luciferase from the mRNA cargo.

FIG. 8 shows LNP-mediated delivery of RNA cargo to the murine liver. Firefly lusciferase mRNA-containing LNPs were formulated and delivered to mice by iv, and liver samples were harvested and assayed for luciferase activity at 6, 24, and 48 hours post administration. Reporter activity by the various formulations followed the ranking LIPIDV005>LIPIDV004>LIPIDV003. RNA expression was transient and enzyme levels returned near vehicle background by 48 hours, post-administration.

FIGS. 9A-9D are a series of diagrams showing exemplary driver constructs and template constructs for plasmid delivery of LTR retrotransposons in trans.

FIG. 10 is a diagram showing integration efficiency measured in HEK293T cells transfected with the indicated driver construct and template construct, as determined by ddPCR.

FIGS. 11A-11B are a series of diagrams showing exemplary constructs for plasmid delivery of LTR retrotransposons in cis. (A) Comparison of a natural LTR retrotransposon (top panel) with an exemplary artificial cis configuration (bottom panel). (B) Three additional exemplary cis configurations, including one with a deletion of the reverse transcriptase/integrase (middle panel) and one with the PBS* modification (bottom panel).

FIG. 12 is a graph showing percentage of GFP+ cells after introduction of a template plasmid carrying a GFP payload and a driver plasmid utilizing an IAP retrotransposon or variants thereof (i.e., a variant with a mutated PBS, “PBS*”; and a variant in which pol was deleted, “IAP Pol Deletion”).

FIG. 13 is a graph showing integration efficiency after introduction of a template plasmid carrying a GFP payload and a driver plasmid utilizing the IAP retrotransposon or variants, as measured by ddPCR.

### DETAILED DESCRIPTION

This disclosure relates to compositions, systems and methods for targeting, editing, modifying or manipulating a DNA sequence (e.g., inserting a heterologous object DNA sequence into a target site of a mammalian genome) at one or more locations in a DNA sequence in a cell, tissue or subject, e.g., in vivo or in vitro. Generally, the systems and compositions include a template RNA comprising a pair of long terminal repeats (LTRs) flanking a heterologous object sequence (e.g., encoding a therapeutic effector). In some instances, the LTRs are derived from a retrovirus (e.g., an endogenous retrovirus). In some instances, the LTRs are derived from a retrotransposon (e.g., an LTR retrotransposon). The template RNA is typically introduced into a target cell with a structural polypeptide domain and a reverse transcriptase polypeptide domain, or nucleic acid molecules encoding the structural polypeptide domain and the reverse transcriptase polypeptide domain. In some instances, the structural polypeptide and/or reverse transcriptase polypeptide domain are derived from a retrovirus (e.g., an endogenous retrovirus). In some instances, the structural polypeptide and/or reverse transcriptase polypeptide domain are derived from a retrotransposon (e.g., an LTR retrotransposon). The template RNA and reverse transcriptase polypeptide domain can be enclosed within a proteinaceous exterior (e.g., a capsid) in the cell, e.g., to form a virus-like particle (VLP). Within the VLP, the reverse transcriptase polypeptide domain can generate a template DNA (e.g., a linear and/or double-stranded DNA) from the template RNA. The template DNA can then optionally be integrated into the genome of the cell, e.g., by an integrase from a retrovirus (e.g., an endogenous retrovirus) or a retrotransposon, e.g., an LTR retrotransposon. The heterologous object sequence may include, e.g., a coding sequence, a regulatory sequence, and/or a gene expression unit.

In some instances, the disclosure provides retrovirus- or retrotransposon-based systems for inserting a sequence of interest into the genome. Additional examples of retrotransposon elements are listed, e.g., in Tables 3A, 3B, 10, 11, X, and Y of PCT Application No. PCT/US2021/020943, and Tables 1 and 2 of PCT Application No. PCT/US2019/048607, each of which applications is incorporated herein by reference in its entirety.

### LTR Retrotransposon Systems

Long terminal repeat (LTR) retrotransposons are a type of mobile genetic elements that are widespread in eukaryotic genomes. Naturally-occurring LTR retrotransposons typically have a coding region flanked by direct (i.e., not inverted) long terminal repeats. The LTR typically includes a promoter whereby the coding region may be transcribed. The coding region typically codes for the Gag and Pol polyproteins. Gag is typically processed by protease to produce structural proteins matrix (MA), capsid (CA), and nucleocapsid (NC) proteins that form the virus-like particle (VLP), and inside of which reverse transcription of the LTR retrotransposon transcript takes place. Pol typically has protease, reverse transcriptase that copies the LTR retrotransposon transcript into cDNA, Rnase H, and integrase, which integrates the cDNA into the host genome. LTR retrotransposons also typically include a primer binding site (PBS) immediately downstream of the 5′LTR and a polypurine tract (PPT) immediately upstream of the 3′LTR.

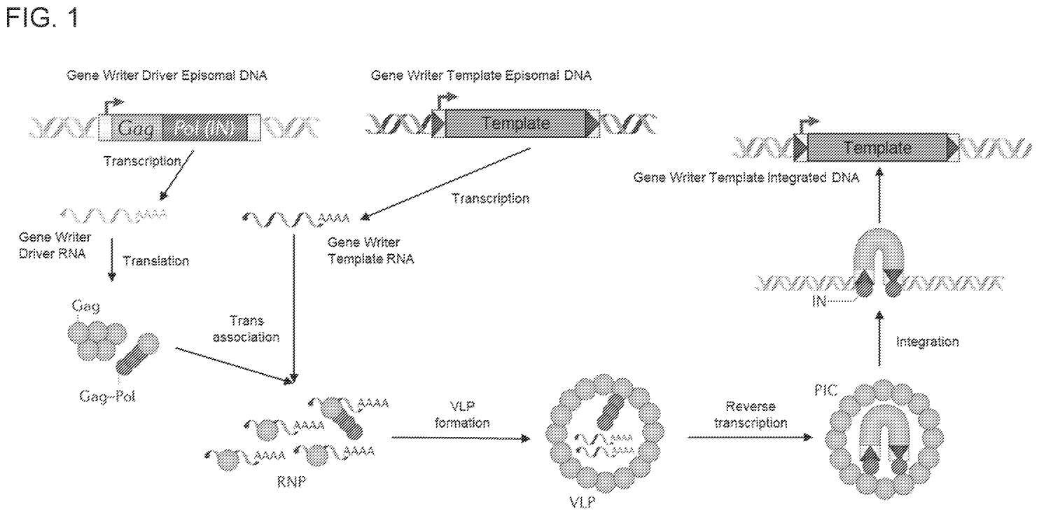

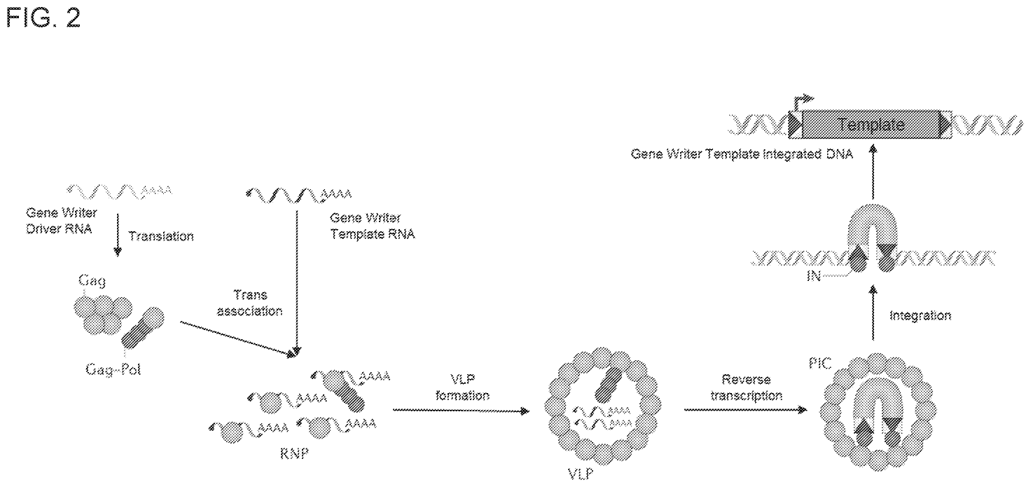

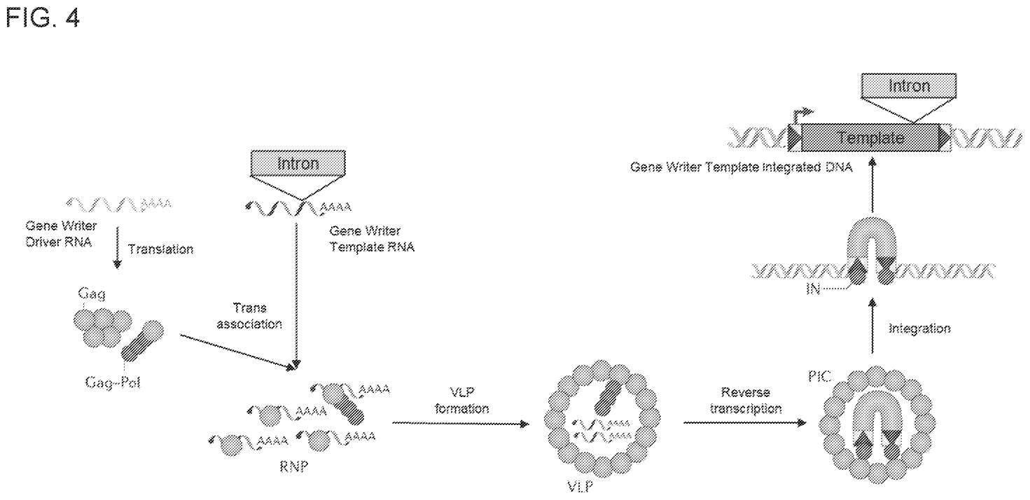

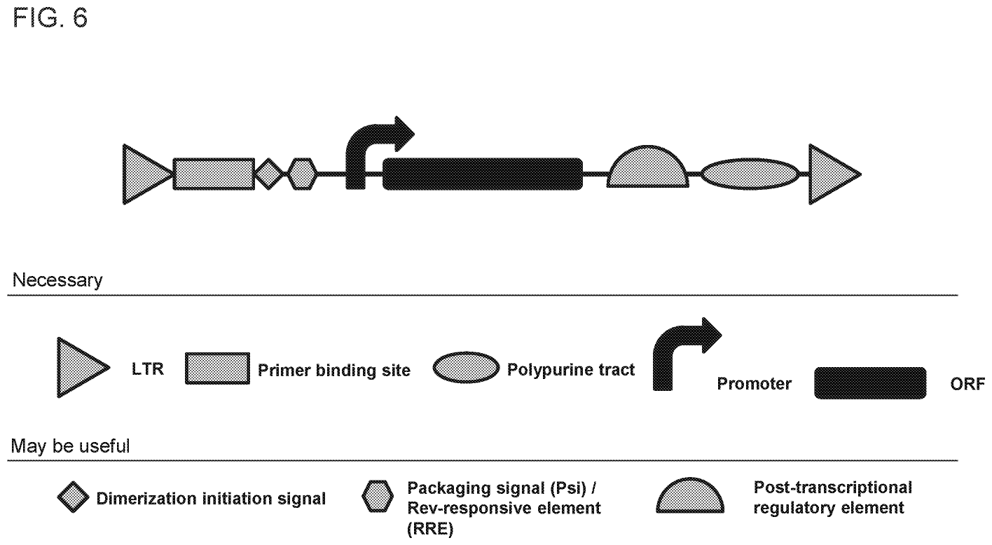

FIG. 1 and FIG. 2 schematically depict systems for integrating a gene of interest in a genome. In both schema, a gene of interest is encoded in a template flanked with LTRs and other components (shown in more detail in FIG. 6). A driver encodes the remaining components of the ERV, retrovirus, or LTR retrotransposon, such as Gag and Pol. The driver and template may be introduced in DNA form (FIG. 1) or RNA form (FIG. 2). If introduced in DNA form, they are transcribed, the driver-derived transcripts are translated to produce the required proteins, which act on the template transcript to produce a cDNA of the template transcript and integrate it into the genome. If introduced in RNA form, the initial transcription step is skipped. A gene of interest may also be introduced with an intron (FIG. 4).

### LTR Retrotransposon and Retroviral-Based Genome Delivery Systems

The present disclosure provides compositions, systems, and methods for integrating a heterologous object sequence (e.g., encoding a therapeutic effector) into the genome of a target cell. Generally, a template RNA is introduced into a cell (e.g., as an RNA molecule, or in the form of a DNA molecule (e.g., an episome) that is transcribed into RNA in the cell). The template RNA is then enclosed in a proteinaceous exterior (e.g., capsid) within the cell, thereby forming a virus-like particle (VLP) in the cell. The template RNA is then reverse-transcribed in the VLP to generate a template DNA, e.g., thereby forming a pre-integration complex (PIC) comprising the template DNA enclosed in the proteinaceous exterior. In some embodiments, the VLP is initially formed in the cytoplasm. In some embodiments, the VLP is initially localized to the endoplasmic reticulum. The VLP does not obtain an envelope. In some embodiments, reverse transcription of the template RNA occurs while the VLP is in the cytoplasm. In some embodiments, reverse transcription of the template RNA occurs while the VLP is in the endoplasmic reticulum or another organeller compartment. In some embodiments, reverse transcription of the template RNA occurs while the VLP is in the nucleus.

Once in the nucleus, the template DNA (or a portion thereof, e.g., the heterologous object sequence) may be integrated into the genome of the cell, e.g., by an integrase (e.g., a retrotransposon integrase or a retroviral integrase, e.g., a lentiviral integrase, e.g., an HIV integrase). In some embodiments, the template DNA is not integrated into the genome of the cell. In certain embodiments, the non-integrated template DNA is circularized, e.g., to form an episome comprising the heterologous object sequence. In some embodiments, the integrated heterologous object sequence may be flanked by one or more LTRs (e.g., the first LTR and/or the second LTR). In some embodiments, the integrant comprises one or more target site duplications (e.g., having a length of about 4, 5, or 6 nucleotides each). In some embodiments, the integration site has one or more (e.g., 1, 2, 3, 4, 5, 6, 7, 8, 9, 10, 11, 12, 13, 14, 15, 16, or all 17) of the following characteristics:
(i) about 1 kb upstream of a gene transcribed by RNA pol III;
(ii) about 2-3 kb (e.g., about 2, 2.5, or 3 kb) upstream of a gene transcribed by RNA pol III;
(iii) comprises a silent mating locus;
(iv) positioned within 10, 20, 30, 40, 50, 60, 70, 80, 90, 100, 500, or 1000 bp of a telomere;
(v) within a promoter, e.g., a promoter for a gene transcribed by RNA pol II;
(vi) within heterochromatin;
(vii) within an enhancer;
(viii) within a transcriptional start site;
(ix) within a gene-rich region of a chromosome;
(x) within a chromosomal region proximal to the nuclear periphery;
(xi) within a nucleosome-free region;
(xii) within a site hypersensitive to DNAse I;
(xiii) located about 40-150 bp (e.g., about 40, 50, 51, 52, 53, 54, 60, 70, 80, 90, 100, 110, 120, 130, 140, or 150 bp of a tRNA coding region);
(xiv) within an exon;
(xv) within an intron;
(xvi) within a gene (e.g., having a parallel orientation to the gene or having an antiparallel orientation to the gene); and/or
(xvii) within a region into which one or more of the following retrotransposons and/or retroviruses is capable of integrating: Ty1, Ty3, Ty5, Tf1, Maggy, MLV, HIV, or PFV.

### Template RNA Component

In some embodiments, the template RNA comprises one or more (e.g., 1, 2, 3, 4, 5, or all 6) of the following (e.g., in order from 5′ to 3′): (i) a first long terminal repeat (LTR), (ii) a primer binding site (PBS), (iii) a promoter, (iv) a heterologous object sequence (e.g., comprising an open reading frame), (v) a polypurine tract, and/or (vi) a second LTR. In some embodiments, the PBS has a length of about 15, 16, 17, 18, 19, or 20 nucleotides (e.g., 18 nucleotides). In some embodiments, the PBS is complementary to a sequence comprised in a tRNA (e.g., a sequence located at the 3′ end of the tRNA) normally provided by the host cell in order to start the reverse transcription. In some embodiments, the polypurine tract (PPT) comprises at least 50%, 75%, 80%, 85%, 90%, 95%, 96%, 97%, 98%, 99%, or 100% A or G nucleotides. The PPT is responsible for starting the synthesis of the proviral (+) DNA strand. In some embodiments, the PPT has a length of about 7, 8, 9, 10, 11, 12, or 13 nucleotides (e.g., 10 nucleotides). In some embodiments, the packaging signal is capable of being specifically bound by a zinc finger protein or a nucleocapsid protein.

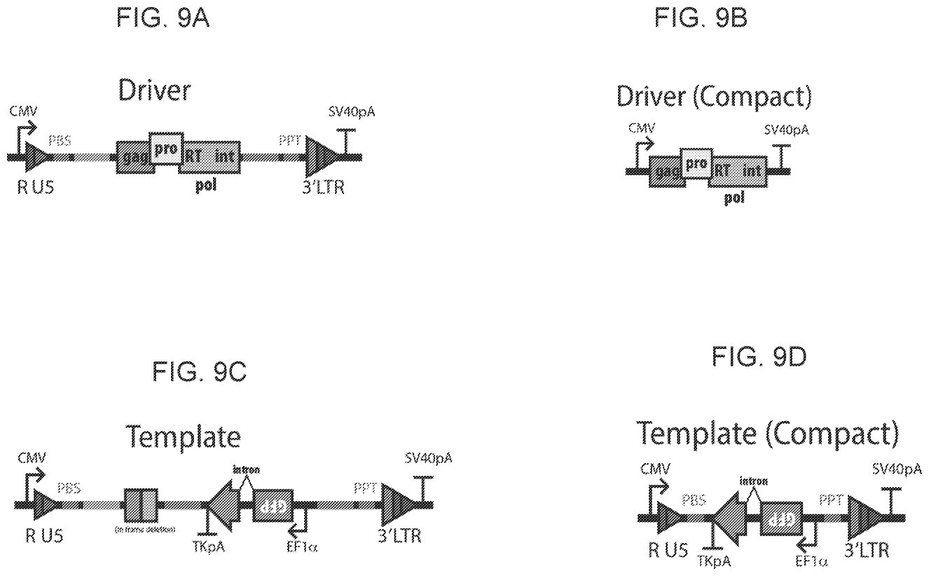

In some embodiments, the template RNA does not comprise a sequence encoding a functional viral protein (e.g., gag, pol, or a viral reverse transcriptase and/or integrase as described herein, or functional fragments thereof). In some embodiments, the template RNA comprises an in-frame deletion of a viral gene, e.g., a gene encoding a functional viral protein (e.g., gag, pol, or a viral reverse transcriptase and/or integrase as described herein, or functional fragments thereof). In some embodiments, the template RNA is introduced into a cell with (e.g., prior to, concurrently with, or after) a driver construct as described herein (e.g., a driver construct comprising one or more genes encoding functional viral proteins, e.g., gag, pol, or a viral reverse transcriptase and/or integrase as described herein, or functional fragments thereof). In some embodiments, a driver construct has a structure as shown in any of FIGS. 9-13. In some embodiments, a template RNA has a structure as shown in any of FIGS. 9-13. In some embodiments, the heterologous object sequence is between the first LTR and the second LTR, and one or more sequences encoding functional viral proteins (e.g., gag, pol, or a viral reverse transcriptase and/or integrase as described herein, or functional fragments thereof) is between the first LTR and second LTR (e.g., between the first LTR and the heterologous object sequence).

In some embodiments, the template RNA comprises one or more sequences encoding a functional viral protein (e.g., gag, pol, or a viral reverse transcriptase and/or integrase as described herein, or functional fragments thereof). In some embodiments, the template RNA comprises a sequence encoding a functional viral gag protein, or a functional fragment thereof. In some embodiments, template RNA comprises a sequence encoding a functional viral pol protein, or a functional fragment thereof. In some embodiments, template RNA comprises a sequence encoding a functional viral reverse transcriptase protein, or a functional fragment thereof. In some embodiments, template RNA comprises a sequence encoding a functional viral integrase protein, or a functional fragment thereof. In certain embodiments, the template RNA comprises a sequence encoding a functional viral gag protein, a functional viral pol protein, and a functional viral reverse transcriptase and/or integrase protein, e.g., as described herein, or functional fragments thereof. In certain embodiments, the sequences encoding functional viral proteins, or functional fragments thereof, are positioned between the primer binding site and the heterologous object sequence. In some embodiments, a template RNA has a structure as shown in any of FIGS. 9-13.

In some embodiments, the first LTR is located at, or within 1, 2, 3, 4, 5, 6, 7, 8, 9, 10, 15, 20, 25, 30, 40, 50, 60, 70, 80, 90, or 100 nucleotides of the 5′ end of the template RNA. In some embodiments, the second LTR is located at, or within 1, 2, 3, 4, 5, 6, 7, 8, 9, 10, 15, 20, 25, 30, 40, 50, 60, 70, 80, 90, or 100 nucleotides of the 3′ end of the template RNA. In some embodiments, one or more of the LTRs has a length of about 100-200, 200-300, 300-400, 400-500, 500-600, 600-700, 700-800, 800-900, 900-1000, 1000-1500, or 1500-2000 nucleotides. In some embodiments, one or more of the LTRs comprises a U3 region (e.g., comprising a promoter). In embodiments, the U3 region is about 200-300, 300-400, 400-500, 500-600, 600-700, 700-800, 800-900, 900-1000, 1000-1100, or 1100-1200 nucleotides. In some embodiments, one or more of the LTRs comprises a repeated region (R). In some embodiments, one or more of the LTRs comprises a U5 region (e.g., having a length of about 75-100, 100-125, 125-150, 150-175, 175-200, 200-225, or 225-250 nucleotides). In some embodiments, one or more of the LTRs comprises a sequence that can be specifically bound by an integrase (e.g., a retroviral or retrotransposon integrase, e.g., as described herein). In embodiments, the sequence that can be specifically bound by an integrase has a length of about 8-10, 10-15, or 15-20 nucleotides. In some embodiments, one or more of the LTRs (e.g., a 5′ LTR) comprises a promoter (e.g., a promoter recognized by PolII). In some embodiments, the LTRs are known as terminal direct repeats or short inverted repeats. In some embodiments the 5′ LTR comprises a R and U5 region and the 3′ LTR comprises a U3 and R region. In some embodiments the 5′ LTR lacks a U3 region and the 3′ LTR lacks a U5 region. In some embodiments. In some embodiments the LTR is a self-inactivating (SIN) LTR that has a ΔU3 modification intended to remove promoter or enhancer activity.

The template RNA of the system typically comprises an object sequence for insertion into a target DNA. The object sequence may be coding or non-coding. In some embodiments, the heterologous object sequence (e.g., of a system as described herein) is about 1-50, 50-100, 100-200, 200-300, 300-400, 400-500, 500-600, 600-700, 700-800, 800-900, 900-1000, 1000-2000, 2000-3000, 3000-4000, 4000-5000, 5000-6000, 6000-7000, 7000-8000, 8000-9000, 9000-10000, or more, nucleotides in length.

In some embodiments, the object sequence may contain an open reading frame. In some embodiments, the template RNA has a Kozak sequence. In some embodiments, the template RNA has an internal ribosome entry site. In some embodiments, the template RNA has a self-cleaving peptide such as a T2A or P2A site. In some embodiments, the template RNA has a start codon. In some embodiments, the template RNA has a splice acceptor site. In some embodiments, splice donor and acceptor sites are removed. In some embodiments, the template RNA has a splice donor site. Exemplary splice acceptor and splice donor sites are described in WO2016044416, incorporated herein by reference in its entirety. Exemplary splice acceptor site sequences are known to those of skill in the art and include, by way of example only, CTGACCCTTCTCTCTCTCCCCCAGAG (SEQ ID NO: 4) (from human HBB gene) and TTTCTCTCCCACAAG (SEQ ID NO: 5) (from human immunoglobulin-gamma gene). In some embodiments the template RNA, has a microRNA binding site downstream of the stop codon. In some embodiments, the template RNA has a polyA tail downstream of the stop codon of an open reading frame. In some embodiments, the template RNA comprises one or more exons. In some embodiments, the template RNA comprises one or more introns. In some embodiments, the template RNA comprises a eukaryotic transcriptional terminator. In some embodiments, the template RNA comprises an enhanced translation element or a translation enhancing element. In some embodiments, the RNA comprises the human T-cell leukemia virus (HTLV-1) R region. In some embodiments, the RNA comprises a posttranscriptional regulatory element that enhances nuclear export, such as that of Hepatitis B Virus (HPRE) or Woodchuck Hepatitis Virus (WPRE). In some embodiments, in the template RNA, the heterologous object sequence encodes a polypeptide and is coded in an antisense direction with respect to the 5′ and 3′ UTR. In some embodiments, in the template RNA, the heterologous object sequence encodes a polypeptide and is coded in a sense direction with respect to the 5′ and 3′ UTR.

In some embodiments, the object sequence may contain a non-coding sequence. For example, the template RNA may comprise a promoter or enhancer sequence. In some embodiments, the template RNA comprises a tissue specific promoter or enhancer, each of which may be unidirectional or bidirectional. In some embodiments, the promoter is an RNA polymerase I promoter, RNA polymerase II promoter, or RNA polymerase III promoter. In some embodiments, the promoter comprises a TATA element. In some embodiments, the promoter comprises a B recognition element. In some embodiments, the promoter has one or more binding sites for transcription factors. In some embodiments, the non-coding sequence is transcribed in an antisense-direction with respect to the 5′ and 3′ UTR. In some embodiments, the non-coding sequence is transcribed in a sense direction with respect to the 5′ and 3′ UTR.

It is understood that, when a template RNA is described as comprising an open reading frame or the reverse complement thereof, in some embodiments the template RNA must be converted into double stranded DNA (e.g., through reverse transcription) before the open reading frame can be transcribed and translated.

In certain embodiments, customized RNA sequence template can be identified, designed, engineered and constructed to contain sequences altering or specifying host genome function, for example by introducing a heterologous coding region into a genome; affecting or causing exon structure/alternative splicing; causing disruption of an endogenous gene; causing transcriptional activation of an endogenous gene; causing epigenetic regulation of an endogenous DNA; causing up- or down-regulation of operably liked genes, etc. In certain embodiments, a customized RNA sequence template can be engineered to contain sequences coding for exons and/or transgenes, provide for binding sites to transcription factor activators, repressors, enhancers, etc., and combinations of thereof. In other embodiments, the coding sequence can be further customized with splice acceptor sites, poly-A tails.

In some embodiments, the template RNA further comprises one or more (e.g., 1, 2, 3, or all 4) of the following: a dimerization initiation signal, a packaging signal (Psi), a Rev-responsive element (RRE), and/or a post-transcriptional regulatory element. A Psi sequence is a Packaging signal that has a secondary RNA structure specifically recognized by either the Zn-fingers or the basic residues of the nucleocapsid domain of the GAG proteins. The PSI sequence is generally located just after the PBS (primer-binding site) but before the Gag AUG. For HIV- and SIV-like retroviruses, the important and selective components of the PSI are an RCC sequence within a 7-base loop, followed or preceded by a less specific GAYC loop with a GC-rich stem (Harrison et al., 1995; Clever et al., 2002). Accessory stem-loop formations ensure a high level of specificity in packaging. A dimerization initiation signal (DIS) triggers dimerization, which allows the recognition and the interaction of the two RNAs, even in the absence of proteins. The signal is formed by a symmetrical loop near the PSI (reviewed by Paillart et al., 2004). This noncovalent, symmetrical intermolecular interaction is called a ‘kissing-loop complex’ for retroviruses, and is further stabilized by a more extended duplex (Paillart et al., 2004, https://www.nature.com/articles/nrimicro90) In a way analogous to that for the PSI sequence, the dimerization mechanism may be specific. Thus, elements of the non-autonomous groups would either harbor the same DIS as their active partners (forming specific heterodimers), or their competitive packaging efficiency must allow them to be preferentially packaged and therefore strictly homodimeric.

FIG. 6 schematically shows the design of a template DNA or RNA. A template typically will contain, in 5′-to-3′ order, a 5′ UTR, a primer binding site, optionally a dimerization initiation signal, optionally a Psi packing signal and/or Rev-responsive element (RRE), a promoter for the gene of interest, the gene of interest, optionally a post-transcriptional regulatory element, a polypurine tract, and a 3′ LTR.

In certain embodiments, the dimerization initiation signal is positioned between the PBS and the promoter. In certain embodiments, the packaging signal (Psi) and/or the RRE are positioned between the dimerization initial signal and the promoter. In certain embodiments, the post-transcriptional regulatory element is positioned between the heterologous object sequence and the polypurine tract. In some embodiments, the template RNA does not comprise a PBS. In some embodiments, the template RNA does not comprise a dimerization initiation signal. In some embodiments, the template RNA does not comprise a packing signal (Psi). In some embodiments, the template RNA does not comprise an RRE. In some embodiments, the template RNA does not comprise a post-transcriptional regulatory element. In some embodiments, the template RNA does not comprise a sequence encoding a structural polypeptide domain (e.g., a gag protein or a functional fragment thereof). In some embodiments, the template RNA does not comprise a sequence encoding a reverse transcriptase polypeptide domain (e.g., a pol protein or a functional fragment thereof).

In some embodiments, the template RNA associates with a protein complex (e.g., comprising gag proteins and/or pol proteins, e.g., a gag-pol polyprotein), e.g., prior to enclosure within the proteinaceous exterior. In some embodiments, the proteinaceous exterior comprises gag proteins and/or pol proteins, e.g., gag-pol polyproteins. In some embodiments, association of the template RNA with the protein complex locally enriches the template RNA for enclosure within the proteinaceous exterior. In some embodiments, the proteinaceous exterior encloses a reverse transcriptase polypeptide domain (e.g., an LTR retrotransposon reverse transcriptase polypeptide domain or a retroviral (e.g., endogenous retroviral) reverse transcriptase polypeptide domain). In some embodiments, the enclosed reverse transcriptase polypeptide domain reverse transcribes the template RNA in the VLP to generate the template DNA. In some embodiments, the proteinaceous exterior encloses an integrase domain (e.g., an LTR retrotransposon integrase domain or a retroviral (e.g., endogenous retroviral) integrase domain). In some embodiments, the enclosed integrase domain integrates the template DNA into the genome of the cell.

In some embodiments, the template RNA comprises a non-canonical RNA. In some embodiments, the template RNA comprises one or more modified nucleobases. In some embodiments, the template RNA is circular. In some embodiments, the template RNA comprises a non-translated cap region. In some embodiments, the template RNA comprises a non-translated tail region (e.g., a poly-A tail).

In some embodiments, the template RNA comprises a ribozyme, e.g., as described in PCT Publication No. WO 2020/142725 (incorporated herein by reference in its entirety). In some embodiments, the ribozyme is capable of self-cleavage (e.g., cleaving the template RNA). In some embodiments, ribozyme self-cleavage results in production of discrete 5′ or 3′ ends. viral RNA genome and subsequent production of infectious RNA viruses. Exemplary ribozymes include, without limitation, the Hammerhead ribozyme (e.g., the Hammerhead ribozymes shown in FIG. 23), the Varkud satellite (VS) ribozyme, the hairpin ribozyme, the GIR branching ribozyme, the glmS ribozyme, the twister ribozyme, the twister sister ribozyme, the pistol ribozyme (e.g., Pistol and Pistol 2 shown in FIG. 24), the hatchet ribozyme, and the Hepatitis delta virus ribozyme. In some embodiments, the template RNA comprises non-viral 5′ and 3′ sequences that enable generation of discrete 5′ and 3′ ends substantially identical to those of a retrovirus or retrotransposon (e.g., as described herein). In some embodiments, the template comprises one or more targeting sites for an endonuclease enzyme (e.g., an RNase, e.g., RNase H), e.g., as described in PCT Publication No. WO 2020/142725, supra. In some embodiments, the template RNA comprises a restriction site that, when cleaved by a restriction enzyme, results in the generation of discrete ends. In embodiments, the template RNA comprises a Type IIS restriction site. Exemplary Type IIS restriction enzymes include, without limitation, Acul, Alwl, Bael, Bbsl, Bbvl, BccI, BceAI, Bcgl, BciVI, BcoDI, BfuAI, Bmrl, Bpml, BpuEI, Bsal, BsaXI, BseRI, Bsgl, BsmAI, BsmBi, Bs F, Bsml, BspCNI, BspMI, BspQI, BsrDI, Bsrl, BtgZI, BtsCI, Bstl, CaspCI, Earl, Ecil, Esp3I, Faul, Fokl, Hgal, Hphl, HpyAV, Mboll, Mlyl, Mmel, MnlL, NmeATTT, Plel, Sapl, and SfaNI.

In some embodiments, the template RNA comprises a sequence encoding an intron (e.g., within the heterologous object sequence). In some embodiments, the intron is integrated into the genome of the cell (e.g., as part of the heterologous object sequence).

In some embodiments, the template RNA comprises a microRNA sequence, a siRNA sequence, a guide RNA sequence, a piwi RNA sequence.

In some embodiments, the template RNA comprises a non-coding heterologous object sequence, e.g., a regulatory sequence. In some embodiments, integration of the heterologous object sequence thus alters the expression of an endogenous gene. In some embodiments, integration of the heterologous object sequence upregulates expression of an endogenous gene. In some embodiments, integration of the heterologous object sequence downregulated expression of an endogenous gene.

In some embodiments, the template RNA comprises a site that coordinates epigenetic modification. In some embodiments, the template RNA comprises an element that inhibits, e.g., prevents, epigenetic silencing. In some embodiments, the template RNA comprises a chromatin insulator. For example, the template RNA comprises a CTCF site or a site targeted for DNA methylation.

In order to promote higher level or more stable gene expression, the template RNA may include features that prevent or inhibit gene silencing. In some embodiments, these features prevent or inhibit DNA methylation. In some embodiments, these features promote DNA demethylation. In some embodiments, these features prevent or inhibit histone deacetylation. In some embodiments, these features prevent or inhibit histone methylation. In some embodiments, these features promote histone acetylation. In some embodiments, these features promote histone demethylation. In some embodiments, multiple features may be incorporated into the template RNA to promote one or more of these modifications. CpG dinculeotides are subject to methylation by host methyl transferases. In some embodiments, the template RNA is depleted of CpG dinucleotides, e.g., does not comprise CpG nucleotides or comprises a reduced number of CpG dinucleotides compared to a corresponding unaltered sequence. In some embodiments, the promoter driving transgene expression from integrated DNA is depleted of CpG dinucleotides.

In some embodiments, the template RNA comprises a gene expression unit composed of at least one regulatory region operably linked to an effector sequence. The effector sequence may be a sequence that is transcribed into RNA (e.g., a coding sequence or a non-coding sequence such as a sequence encoding a micro RNA).

In some embodiments, the object sequence of the template RNA is inserted into a target genome in an endogenous intron. In some embodiments, the object sequence of the template RNA is inserted into a target genome and thereby acts as a new exon. In some embodiments, the insertion of the object sequence into the target genome results in replacement of a natural exon or the skipping of a natural exon.

In some embodiments, the object sequence of the template RNA is inserted into the target genome in a genomic safe harbor site, such as AAVS1, CCR5, or ROSA26. In some embodiments, the object sequence of the template RNA is inserted into the albumin locus. In some embodiments, the object sequence of the template RNA is inserted into the TRAC locus. In some embodiments, the object sequence of the template RNA is added to the genome in an intergenic or intragenic region. In some embodiments, the object sequence of the template RNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments, the object sequence of the template RNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments, the object sequence of the template RNA can be, e.g., 50-50,000 base pairs (e.g., between 50-40,000 bp, between 500-30,000 bp between 500-20,000 bp, between 100-15,000 bp, between 500-10,000 bp, between 50-10,000 bp, between 50-5,000 bp. In some embodiments, the heterologous object sequence is less than 1,000, 1,300, 1500, 2,000, 3,000, 4,000, 5,000, or 7,500 nucleotides in length.

In some embodiments the template RNA has a poly-A tail at the 3′ end. In some embodiments the template RNA does not have a poly-A tail at the 3′ end.

In some embodiments a system or method described herein comprises a single template RNA. In some embodiments a system or method described herein comprises a plurality of template RNAs. In some embodiments, when the system comprises a plurality of nucleic acids, one or more nucleic acid comprises a conjugating domain. In some embodiments, a conjugating domain enables association of nucleic acid molecules, e.g., by hybridization of complementary sequences.

In some embodiments, the template (e.g., template RNA) comprises certain structural features, e.g., determined in silico. In embodiments, the template RNA is predicted to have minimal energy structures between −280 and −480 kcal/mol (e.g., between −280 to −300, −300 to −350, −350 to −400, −400 to −450, or −450 to −480 kcal/mol), e.g., as measured by RNAstructure, e.g., as described in Turner and Mathews Nucleic Acids Res 38:D280-282 (2009) (incorporated herein by reference in its entirety).

In some embodiments, the template (e.g., template RNA) comprises certain structural features, e.g., determined in vitro. In embodiments, the template RNA is sequence optimized, e.g., to reduce secondary structure as determined in vitro, for example, by SHAPE-MaP (e.g., as described in Siegfried et al. Nat Methods 11:959-965 (2014); incorporated herein by reference in its entirety). In some embodiments, the template (e.g., template RNA) comprises certain structural features, e.g., determined in cells. In embodiments, the template RNA is sequence optimized, e.g., to reduce secondary structure as measured in cells, for example, by DMS-MaPseq (e.g., as described in Zubradt et al. Nat Methods 14:75-82 (2017); incorporated by reference herein in its entirety).

It is understood that in referring to nucleotide distances between elements in nucleotides, unless specified otherwise, distance refers to the number of nucleotides (of a single strand) or base pairs (in a double strand) that are between the elements but not part of the elements. As an example, if a first element occupies nucleotides 1-100, and a second element occupies nucleotides 102-200 of the same nucleic acid, the distance between the first element and the second element is 1 nucleotide.

### Polypeptide Components

Gag. Gag is processed by protease into matrix (MA), capsid (CA), and nucleocapsid (NC) proteins. MA is necessary for membrane targeting of gag polyprotein and for capsid assembly. Matrix interacts with viral membrane. CA forms the prominent hydrophobic core of the virion. (viral capsid). The best-conserved part of the gag polyprotein is the CA-like major homology region (MHR), which usually displays a central QG-X2-E-X5-F-X2-L-X2-H motif (SEQ ID NO: 6) implicated in the transposition. NC is involved in RNA packaging through recognition of a specific region of the viral genome called Ψ (PSI genome packaging). A second similarity within gag polyproteins is found in the C-terminus of the NC as a Cys-X2-Cys-X4-His-X4-Cys (CCHC) motif (SEQ ID NO: 7), which may be absent or found one, two, or three times duplicated depending on the viral species. CCHC arrays have been found to be critical for many steps in the viral life cycle, and several studies have shown they are involved in virion assembly, RNA packaging, reverse transcription, and integration processes. Each CCHC motif coordinates a zinc atom. Gag may lack Matrix in some cases, e.g. Ty3 (https://onlinelibrary.wiley.com/doi/abs/10.1128/9781555819217.ch42). Gag may lack NC in some cases, e.g., Ty1. Gag in LTR retrotransposons typically lacks functional sequence for myristoylation and plasma membrane targeting (Ribet al 2006). In the systems described herein, therefore, gag sequence can be taken from ERVs or retroviruses with myristoylation knocked out.

Pol. Pol translation can be mediated by several mechanisms. For examples, the retrotransposon may include an internal ribosome entry site (IRES) for Pol. The sequence between Gag and Pol ORFs may include a small repetitive motif (such as AAAAA) that induces slippage of the ribosome, which then allows the translation of the second ORF by frameshifting. Another possible means is the use of a specific and rare transfer RNA (tRNA), causing ribosomal stalling and slippage and allowing entry into the second ORF. Gag and Pol may also occue in a ORF along with gag. The component proteins of Pol may occur in various orders (e.g., TY1/Copia like: PR-INT-RT-RH; TY3/Gypsy like: PR-RT-RH-INT). They may also be frameshifted from each other, as in intracisternal A particle (IAP) elements.

Protease. Proteases (PR) play a key role in the maturation process during which several peptides involved in the life cycle of the retroelement are scissed by this enzyme. LTR retroelement PRs belong to clan AA of aspartic peptidases. They dimerize in their active form and may be encoded as a part of the pol polyprotein, alone or as a part of the gag polyprotein, or in frame with a dUTPase. It is well known that the structural PR homodomain is founded in a core ˜90-150 residues long wherein the catalytic DTG motif is the most prominent feature along with a glycine at the C-terminal end preceded by two hydrophobic residues. At the primary structure level the most conserved part (core) of all clan peptidases may be divided in six amino acidic patterns constituting a template we have called “DTG/ILG”. The “DTG/ILG” template is the primary structure phenotype of a structural supersecondary structure, called “Andreeva's” template (Andreeva 1991) that was previously used to describe pepsins and retropepsin. The “Andreeva's” template is constituted by the following structural elements: an N-terminal loop (A1), a loop containing the catalytic motif (B1), an α-helix (C1) usually not preserved in retropepsins, a β-hairpin loop (D1), a hairpin loop (A2), a wide loop (B2), an α-helix (C2) towards C-terminal, and a loop (D2), which in empirically characterized retropepsins is substituted by a strand or a helical turn (Wlodawer and Gustchina 2000; Dunn et al. 2002). These elements are responsible of keep both function and three-dimensional (3D) structure in characterized retropepsins and other characterized clan AA peptidases (Wlodawer and Gustchina 2000; Dunn et al. 2002). It has also recently suggested that the structure of the HIV-1 (see the figure below) and other clan AA PRs have a flexibility-assisted mechanism evolutionarily preserved to favor the reactive conformation of the enzyme (Piana, Carloni, and Rothlisberger 2002; Piana, Carloni, and Parrinello 2002; Perryman, Lin, and McCammon 2004).

Reverse transcriptase. The Reverse Transcriptase (RT) is an enzyme capable of catalyzing the synthesis of DNA from a single strain of RNA or DNA. The reverse-transcription process is common among a wide range of prokaryotic and eukaryotic mobile genetic elements, and requires a primer of 12-18 bases in length usually provided by the 3′end of a host tRNA. At the primary structure level, RTs codified by Ty3 Gypsy and Retroviridae elements expand approximately 350 residues of the pol polyprotein, including an alignable core of approximately 180 aa wherein seven conserved regions can be distinguished. At the three-dimensional (3D) structure level the RT codified by the HIV-1 retrovirus is an asymmetrical heterodimer composed of two subunits of 66 and 51 kDa, p66 and p51 respectively. P66 can be divided into five structural subdomains consisting in the RNaseH domain and four subdomains which, due to their similarity to a human right hand, are referred to as fingers, palm, thumb, and connection (Kohlstaedt et al. 1992). P51 is a p-66′ derivative after proteolytic processing and excision of the RNase H. Although several evidences indicate that RTs encoded by other vertebrate retroviruses also form a heterodimer, the RT may also be functionally active as a monomer

Ribonuclease H. Ribonuclease H (RNase H) is a hydrolytic enzyme widely distributed in both prokaryotes and eukaryotes (Johnson et al. 1986; Doolittle et al. 1989). In Ty3 Gypsy and Retroviridae and other LTR retroelements this enzyme is encoded as a part of the pol polyprotein and constitutes the C-terminal end of the Reverse Transcriptase (RT). RNase H is responsible for the hydrolysis of the original RNA template that is part of the RNA/DNA hybrid generated after the retrotranscription process in the viral life cycle. The three dimensional (3D) structure of the HIV-1 RNase H is characterized by four or five α-helices and five β-sheets that interact aligning in parallel to conform the active site (Davies et al. 1991). The activity of this enzyme normally requires the presence of divalent cations like Mg2+ or Mn2+ that bind to an active site constituted by a catalytic triad (Asp-443-Glu-478-Asp-498). These three residues have been proposed to be important in RNase H-mediated catalysis by HIV-1 RT (Mizarhi et al. 1990; Davies et al. 1991). Mutations in any of these resides inhibit the RNase H activity but have small effects on polymerase activity of the HIV-1 retrovirus (Schatz et al. 1989; Mizarhi et al. 1990; Davies et al. 1991; Destefano et al. 1994).

Integrase. Retroelement integrases (INTs) are zinc finger nucleic acid-processing enzymes that catalyze the insertion of reverse-transcribed retroviral DNA into the host genome (Chiu and Davies 2004; Nowotny 2009). These enzymes remove two bases from the end of the LTR and are responsible for the insertion of the linear double-stranded viral DNA copy into the host cell DNA. INT amino acid architecture includes three subdomains: (a) The N-terminal subdomain, which displays a conserved Zinc finger “HHCC” binding motif (Lodi et al. 1995); (b) The central subdomain, which contains a catalytic core characterized by the presence of a conserved D-D-E motif (Kan et al. 1991; Polard and Chandler 1995); and (c) The C-terminal subdomain, which is less preserved than the others. INT enzyme seems to be related to unspecific DNA-binding although several studies of chimeric integrases assign this function to the central core (Katzman and Sudol 1995; Shibagaki and Chow 1997), while other authors alternatively suggest that the C-terminal subdomain might interact with a sub-terminal region of the viral DNA (Jenkins et al. 1997; Heuer and Brown 1997; Esposito and Craigie 1998; Heuer and Brown 1998). The functional structure of LTR retroelement-like INTs is already under study although it seems to be, together with a proviral DNA molecule and other viral and host proteins, part of a pre-integration complex of which little is known. Several studies suggest that this enzyme could act as a multimer or at least as a dimer (for a review in this topic see Craigie 2001).

Chromodomain. LTR retrotransposons may include a Chromatin Organization Modifier Domain (chromodomain). The chromodomain is a protein domain of approximately 50 residues in length, originally identified as a motif common to the Drosophila chromatin proteins Polycomb (Pc) and the heterochromatin protein1 HP1. Chromodomains are involved in chromatin remodeling and regulation of the gene expression in eukaryotes (Koonin, Zhou and Lucchesi 1995; Cavalli and Paro 1998). Almost but not all elements belonging to a lineage ofMetaviridae Ty3 Gypsy LTR retrotransposons described in the genomes of plants, fungi, and vertebrates, are carriers of a chromodomain displayed at the C-terminal end of their integrases (Malik and Eickbush 1999).

dUTPase. dUTPases (DUTs) are cellular enzymes closely similar to Uracil-DNA glycosylases and that hydrolyze dUTP to dUMP and PPi, providing a substrate for thymidylate synthase (an enzyme that converts dUMP to TMP). The expression of cellular DUTs is regulated by the cell cycle; at high levels in dividing undifferentiated cells; and at low levels in terminally non-dividing differentiated cells (Miller et al. 2000). Certain retroviral lineages such as non-primate lentiviruses, betaretroviruses, and ERV-L elements encode and package DUTs into virus particles. However, depending on the genus, the dut gene is located in different zones of the internal region. While betaretroviruses codify for this enzyme in frame and N-terminal to the protease domain, lentiviruses and ERV-L elements present the ORF of this gene between or downstream to the RNaseH and INT domains (Elder et al. 1992; Turelli et al. 1997; Payne and Elder 2001 and references therein). In lentiviruses, DUT facilitates viral replication in non-dividing cells and prevents accumulation of G-to-A transitions in the viral genome, the role of DUT in betaretroviruses and ERV-L elements is still unclear. DUTPase domains have been also described in the genome of some Ty3 Gypsy LTR retrotransposons (Novikova and Blinov 2008) as well as in that of two plant paretroviruses belonging to Badnavirus genus [Dioscorea bacilliform virus (DBV) and Taro bacilliform virus (TaBV)].

In some embodiments, one or more of the gag, pol, gag-pol, reverse transcriptase polypeptide domain, and/or integrase domain are derived from an LTR retrotransposon, e.g., as described herein. In some embodiments, one or more of the gag, pol, gag-pol, reverse transcriptase polypeptide domain, and/or integrase domain are derived from a retrovirus (e.g., a an endogenous retrovirus), e.g., as described herein. In some embodiments, one or more of the gag, pol, gag-pol, reverse transcriptase polypeptide domain, and/or integrase domain are derived from an endogenous retrovirus, e.g., as described herein. In some embodiments, one or more of the gag, pol, gag-pol, reverse transcriptase polypeptide domain, and/or integrase domain are introduced into the cell as proteins. In some embodiments, one or more of the gag, pol, gag-pol, reverse transcriptase polypeptide domain, and/or integrase domain are introduced into the cell as RNA (e.g., mRNA that is translated to produce the proteins). In some embodiments, one or more of the gag, pol, gag-pol, reverse transcriptase polypeptide domain, and/or integrase domain are introduced into the cell as DNA (e.g., a plasmid or episome), e.g., wherein genes encoding the gag, pol, gag-pol, reverse transcriptase polypeptide domain, and/or integrase domain are transcribed from the DNA and the resultant mRNA subsequently translated to produce the protein. In some embodiments, one or more of the gag, pol, gag-pol, reverse transcriptase polypeptide domain, and/or integrase domain is introduced into the cell by electroporation. In some instances, one or more of the gag, pol, gag-pol, reverse transcriptase polypeptide domain, and/or integrase domain is introduced into the cell via a lipid nanoparticle (LNP).

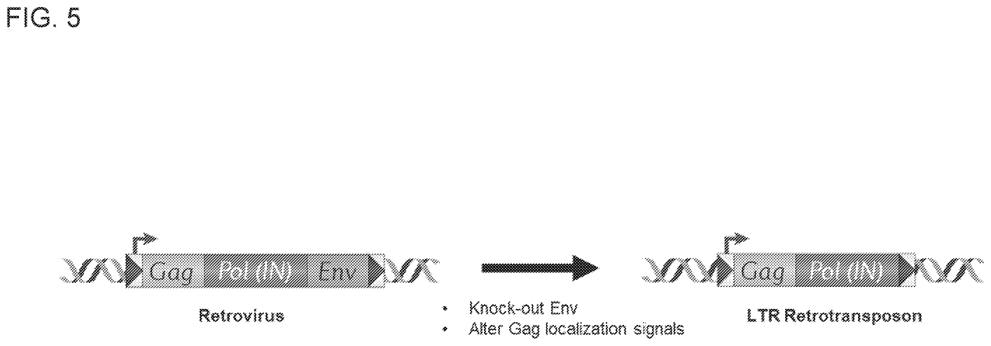

In some embodiments, a nucleic acid molecule (e.g., a DNA or RNA) encoding one or more of the gag, pol, gag-pol, reverse transcriptase polypeptide domain, and/or integrase domain does not comprise a sequence encoding an Env protein (e.g., as described in Magiorkinis et al. 2012, PNAS 109(19) 7385-7390; incorporated herein by reference in its entirety). In some embodiments, the cell does not comprise an Env protein or any nucleic acid molecules encoding an Env protein. In some embodiments, the gag, pol, gag-pol, reverse transcriptase polypeptide domain, and/or integrase domain are derived from a retrovirus, which has been engineered to remove the Env protein and/or to remove a nucleic acid sequence encoding the Env protein (e.g., to produce an LTR retrotransposon). In some embodiments, the gag, pol, gag-pol, reverse transcriptase polypeptide domain, and/or integrase domain are derived from a retrovirus that has been rendered nontransferable, e.g., via 5-azacytidine. In some embodiments, the gag, pol, gag-pol, reverse transcriptase polypeptide domain, and/or integrase domain are derived from a retrovirus that has been engineered to delete a myristoylation signal in the gag protein (or a functional fragment thereof). In some embodiments, the gag, pol, gag-pol, reverse transcriptase polypeptide domain, and/or integrase domain are derived from a retrovirus that has been engineered to remove a signal sequence for plasma membrane targeting. In some embodiments, the gag, pol, gag-pol, reverse transcriptase polypeptide domain, and/or integrase domain are derived from a retrovirus that has been engineered to modify the localization signal in the gag protein (or a functional fragment thereof), e.g., such that the gag protein or functional fragment thereof remains in the cell and/or localizes to the endoplasmic reticulum (e.g., FIG. 5).

In some embodiments, the structural polypeptide domain comprises a gag polyprotein, or a functional fragment (e.g., domain) thereof (e.g., a P24, P17, or P7/P9 domain). In some embodiments, the structural polypeptide domain lacks a myristoylation sequence. In some embodiments, the structural polypeptide domain lacks a plasma membrane targeting sequence. In some embodiments, the structural polypeptide domain comprises a matrix (MA) protein (e.g., a P17 protein). In some embodiments, the structural polypeptide domain comprises a capsid (CA) protein (e.g., a P24 protein). In some embodiments, the structural polypeptide domain comprises a nucleocapsid (NC) protein (e.g., a P7/P9 protein). In some embodiments, the structural polypeptide domain does not comprise a matrix protein. In some embodiments, the structural polypeptide domain does not comprise a nucleocapsid protein.

In some embodiments, the reverse transcriptase polypeptide domain comprises a pol polyprotein, or a functional fragment (e.g., domain) thereof (e.g., an RT, IN, PR, or DU domain). In some embodiments, the reverse transcriptase polypeptide domain comprises a retroviral or retrotransposon reverse transcriptase (RT). In some embodiments, the reverse transcriptase polypeptide domain comprises a retroviral or retrotransposon protease (PR). In some embodiments, the reverse transcriptase polypeptide domain comprises a retroviral or retrotransposon integrase (IN). In some embodiments, the reverse transcriptase polypeptide domain comprises a retroviral or retrotransposon dUTPase (DU). In some embodiments, the reverse transcriptase polypeptide domain comprises a RNase H. In some embodiments, the reverse transcriptase polypeptide domain comprises a chromodomain. In some embodiments, the reverse transcriptase polypeptide domain does not comprise a chromodomain.

In some embodiments, the structural polypeptide domain and the reverse transcriptase polypeptide domain are part of the same polypeptide (e.g., a gag-pol). In some embodiments, the structural polypeptide domain and the reverse transcriptase polypeptide domain are different polypeptides. In some embodiments, the structural polypeptide domain and the reverse transcriptase polypeptide domain are encoded by the same nucleic acid molecule (e.g., comprising an internal ribosome entry site (IRES) between the sequences encoding the structural polypeptide domain and the reverse transcriptase polypeptide domain).

In some embodiments, a system or composition as described herein comprises elements (e.g., polypeptides or nucleic acid molecules) derived from a retrotransposon (e.g., an LTR retrotransposon). Non-limiting examples of retrotransposons that may be used as described herein include MusD, Gypsy/Ty3 (clades CRM, Del, Galadriel, Reina, REM1, G-Rhodo, Pyggy, MGLR3, Pyret, Maggy, MarY1, Tse3, TF1-2, Ty3, V-clade, Skipper, Athila, Tat, 17.6, Gypsy, 412/mdg1, Micropia/mdg3, A-clade, B-clade, C-clade, Gmr1, Osvaldo, Cer2-3, Cer1, CsRN1, Tor1, Tor4, Tor2, and Cigr-1), Copia/Ty1 (clades Ty (Pseudovirus), CoDi-I or CoDi-A, CoDi-II or CoDi-B, CoDi-C, CoDi-D, GalEA, p-Cretro, Sire, Oryco, Retrofit, Tork, Osser, PyREIG1, Hydra, Copia, 1731, Tricopia, Mtanga, and Humnum), Copia/Ty1 (clades Ty (Pseudovirus), CoDi-I or CoDi-A, CoDi-II or CoDi-B, CoDi-C, CoDi-D, GalEA, p-Cretro, Sire, Oryco, Retrofit, Tork, Osser, PyREIG1, Hydra, Copia, 1731, Tricopia, Mtanga, and Humnum), Bel/Pao, Morgane, BARE2, Large Retrotransposon Derivative (LARD), Terminal-repeat Retrotransposon in Miniature (TRIM), IAP, and ETn. In some embodiments, a system or composition as described herein comprises elements of an LTR retrotransposon derived from a rodent (e.g., a rodent of family Muridae, e.g., a mouse).

In some embodiments, a system or composition as described herein comprises elements (e.g., polypeptides or nucleic acid molecules) derived from a MusD retrotransposon (e.g., a U3, R, U5, 5′ LTR, 3′ LTR, PBS, gag, pro, pol, 5′ flank, 3′ flank, PBS*, or PPT element of a MusD retrotransposon, e.g., as described herein, e.g., in Table S2). In certain embodiments, a system or composition as described herein comprises elements derived from a MusD retrotransposon as described in Ribet et al. (2004, Genome Res. 14: 2261-2267; incorporated herein by reference in its entirety). In certain embodiments, a system or composition as described herein comprises elements (e.g., polypeptides or nucleic acid molecules) derived from a MusD1 retrotransposon (e.g., a sequence as listed in Table S2 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from a MusD2 retrotransposon (e.g., a sequence as listed in Table S2 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from a MusD3 retrotransposon (e.g., a sequence as listed in Table S2 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from a MusD4 retrotransposon (e.g., a sequence as listed in Table S2 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from a MusD5 retrotransposon (e.g., a sequence as listed in Table S2 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from a MusD6 retrotransposon (e.g., a sequence as listed in Table S2 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from a MusD7 retrotransposon (e.g., a sequence as listed in Table S2 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from a MusD8 retrotransposon (e.g., a sequence as listed in Table S2 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from a MusD9 retrotransposon (e.g., a sequence as listed in Table S2 or S5, or a sequence having at least 75%, 80%, 85%, 90%, 95%, 96%, 97%, 98%, or 99% sequence identity thereto).

In some embodiments, a system or composition as described herein comprises elements (e.g., polypeptides or nucleic acid molecules) derived from an ETnII retrotransposon (e.g., a U3, R, U5, 5′ LTR, 3′ LTR, PBS, 5′ flank, 3′ flank, or PPT element of a ETnII retrotransposon, e.g., as described herein, e.g., in Table S3). In certain embodiments, a system or composition as described herein comprises elements derived from an ETnII retrotransposon as described in Ribet et al. (2004, Genome Res. 14: 2261-2267; incorporated herein by reference in its entirety). In certain embodiments, a system or composition as described herein comprises elements (e.g., polypeptides or nucleic acid molecules) derived from an ETnII A1 retrotransposon (e.g., a sequence as listed in Table S3 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from an ETnII B1 retrotransposon (e.g., a sequence as listed in Table S3 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from an ETnII B2 retrotransposon (e.g., a sequence as listed in Table S3 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from an ETnII B3 retrotransposon (e.g., a sequence as listed in Table S3 or S5, or a sequence having at least 75%, 80%, 85%, 90%, 95%, 96%, 97%, 98%, or 99% sequence identity thereto).

In some embodiments, a system or composition as described herein comprises elements (e.g., polypeptides or nucleic acid molecules) derived from an ETnI 1 retrotransposon (e.g., a sequence as listed in Table S3 or S5, or a sequence having at least 75%, 80%, 85%, 90%, 95%, 96%, 97%, 98%, or 99% sequence identity thereto). In certain embodiments, a system or composition as described herein comprises elements derived from an ETnI retrotransposon as described in Ribet et al. (2004, Genome Res. 14: 2261-2267; incorporated herein by reference in its entirety).

In some embodiments, a system or composition as described herein comprises elements (e.g., polypeptides or nucleic acid molecules) derived from an IAP retrotransposon (e.g., a U3, R, U5, 5′ LTR, 3′ LTR, PBS, PBS*, gag, pro, or pol element of an IAP retrotransposon, e.g., as described herein, e.g., in Table S4). In certain embodiments, a system or composition as described herein comprises elements derived from an IAP retrotransposon as described in Dewannieux et al. (2004, Nat. Genetics 36(5): 534-539; incorporated herein by reference in its entirety). In certain embodiments, a system or composition as described herein comprises elements (e.g., polypeptides or nucleic acid molecules) derived from an IAP-RP23 retrotransposon (e.g., a sequence as listed in Table S4 or S5, or a sequence having at least 75%, 80%, 85%, 90%, 95%, 96%, 97%, 98%, or 99% sequence identity thereto). In certain embodiments, a system or composition as described herein comprises elements (e.g., polypeptides or nucleic acid molecules) derived from an IAP-92L23 retrotransposon (e.g., a sequence as listed in Table S4 or S5, or a sequence having at least 75%, 80%, 85%, 90%, 95%, 96%, 97%, 98%, or 99% sequence identity thereto).

In some embodiments, the retrotransposon comprises a DIRS element. DIRS elements encode tyrosine recombinase (YR) to perform genome integration, which is the feature the most distinguishing feature from other LTR retrotransposons. YR-encoding retroelements can be classified in 3 groups: (a) DIRS-like: A sub-group of YR elements phylogenetically close to the DIRS1 retrotransposon from Dictyostelium; (b) Ngaro-like: A sub-group of YR elements phylogenetically close to DrNgaro1 from Danio rerio; and (c) PAT-like: A sub-group of YR elements phylogenetically close to PAT from Panagrellus. DIRS elements may have three long ORFs: ORF1 (putative gag-like), ORF2 (tyrosine recombinase or YR ORF) and ORF3 (reverse transcriptase/RNAaseH/N6 deoxy-adenosine methylase or RT/RH/DAM ORF). Portions of the ORFs may overlap. The uncorrupted YR ORFs of all the full-length DIRS-like, PAT-like and Ngaro-like retroelements encode proteins bearing highly conserved RHRY tetrads similar to those of tyrosine recombinases. Templates based on DIRS may have, e.g., terminal inverted repeats (ITRs) that may be non-identical, and/or an internal complementary region, with sequence that is complementary to portions of one or both ITRs. An internal complementary region may be a circular junction. In certain embodiments, systems using portions of DIRS elements do generate a target-site duplication. For example, the recombination of a circular DNA into the genome using a site-specific recombinase may not generate a target site duplication. Exemplary DIRS elements are identified in http://www.biomedcentral.com/1471-2164/12/621. A functional study of DIRS elements (doi: 10.1093/nar/gkaa160) reported that DIRS-1 produces a mixture of single-stranded, mostly linear extrachromosomal cDNA intermediates and that if this cDNA is isolated and transformed into D. discoideum cells, it can be used by DIRS-1 proteins to complete productive retrotransposition.

In some embodiments, a system or composition as described herein comprises elements (e.g., polypeptides or nucleic acid molecules) derived from a retrovirus (e.g., an endogenous retrovirus). Non-limiting examples of retroviruses that may be used as described herein include: lentivirus (e.g., an HIV, e.g. HIV-1 or HIV-2), metavirus, pseudovirus, belpaovirus, betaretrovirus, picornavirus (e.g., enterovirus, e.g., enterovirus 71, coxsackievirus A16, or poliovirus), hepatovirus (e.g., a hepatitis virus, e.g., hepatitis A virus), calcivirus (e.g., norovirus or vesivirus), alphavirus (e.g., Semliki Forest virus, Sindbis virus, and Venezuelan equine encephalitis virus), flavivirus (e.g., Kunjin virus, yellow fever virus, West Nile virus, dengue virus, Zika virus, encephalitis virus, or hepacivirus, e.g., hepatitis C virus), coronavirus (e.g., murine hepatitis virus, SARS-CoV, or SARS-CoV-2), hepevirus (e.g., hepatitis E virus), reovirus, birnavirus (e.g., avibirnavirus), arenavirus, and vesicular stomatitis virus.

In some embodiments, the system comprises an inhibitor of one or more retrovirus restriction factors, including APOBEC3, APOBEC3G (Esnault et al., Nature 433, 2005), APOBE3G, APOBEC3F, APOBEC3, AID (activation induced deaminase doi:10.1093/nar/gk1054), APOBEC3A (DOI 10.1016/j.cub.2006.01.031), APOBEC3B (doi:10.1093/nar/gkj416), APOBEC1 (doi:10.1093/nar/gkr124), Dnmt, Dnmt1, Dnmt1o, Dnmt3a, Dnmt3b, Dnmt31, Edg2, Fv1, Mst1r, Fv4, Fv5, Lsh, Nxf1, Ref1/lv1/Trim5, Rfv1/2/3, Rmcf1, Rmv1/2/3, Slc20a2, Xpr1, and ZAP) and/or comprises one or more retroviral accessory genes (e.g., vpr, vif), to promote replication.

### Integration-Deficient Systems

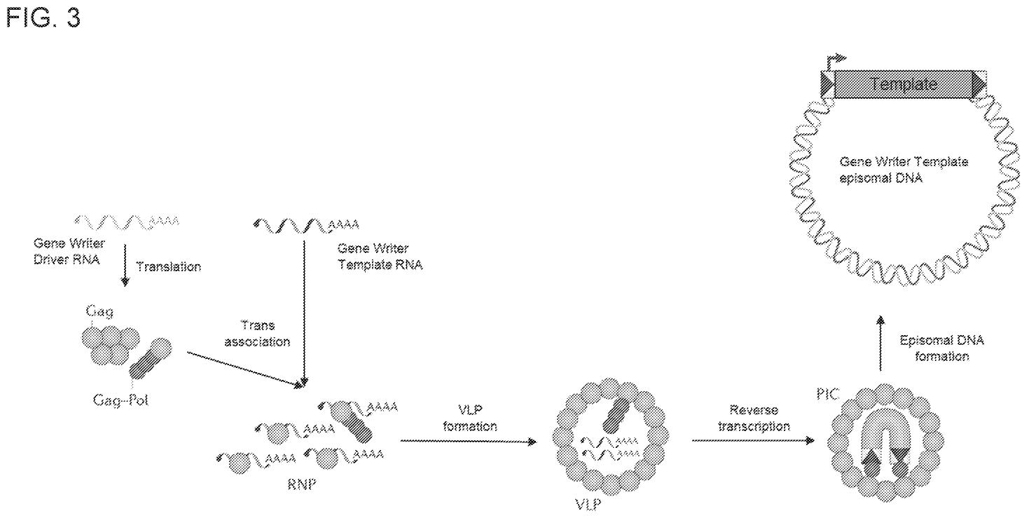

The retroviral or retrotransposon systems described herein may, in some instances, be integration-deficient. In some embodiments, the integrase of the retrovirus or retrotransposon is substantially unable to integrate the template DNA into a target DNA (e.g., a genomic DNA). In some embodiments, the retroviral or retrotransposon system is integration-deficient independent of host cell repair machinery. In some embodiments, the retroviral or retrotransposon system is integration-deficient independent of a transposase, recombinase, and/or nuclease of the host cell. In embodiments, the integrase of the retrovirus or retrotransposon has reduced integrase activity, e.g., to at least 50%, 40%, 30%, 20%, 10%, 5%, 2%, or 1% of that of a corresponding wild-type sequence, e.g., as measured in an assay as described in Moldt et al. 2008 (BMC Biotechnol. 8:60; incorporated herein by reference). In some embodiments, the integrase of the retrovirus or retrotransposon comprises a mutation that reduces integrase activity, e.g., to at least 50%, 40%, 30%, 20%, 10%, 5%, 2%, or 1% of a corresponding wild-type sequence (e.g., a class I mutation, e.g., a mutation in a catalytic triad residue, such as mutations corresponding to D64, D116, and E152 for HIV-1 integrase). In some embodiments, one or both of the U3 and U5 attachment (att) sites at either end of the element may be mutated or deleted to impair integrase binding. In some embodiments, the system comprises an inhibitor (e.g., a small molecule inhibitor) of the integrase of the retrovirus or retrotransposon. Examples of inhibitors include, for HIV-1, strand-transfer inhibitors raltegravir elvitegravir. In some embodiments, the template RNA and/or template DNA does not comprise a DNA recognition site bound by and/or recognized by the integrase of the retrovirus or retrotransposon. ERVs, retrovirus, and LTR transposons engineering to be episomal are shown schematically in FIG. 3.

### Episomes

In some embodiments, an LTR retrotransposon-based system or method described herein can produce an episome (e.g., an episome comprising a heterologous object sequence), a circular DNA molecule. In some embodiments, an episome produced by a system or method described herein comprises an LTR. In certain embodiments, an episome produced by a system or method described herein comprises a plurality of LTRs (e.g., two LTRs). In some embodiments, an episome (e.g., an episome comprising two LTRs) is formed by non-homologous end joining (NHEJ), e.g., ligating together the 5′ and 3′ ends of a linear DNA (e.g., a vector DNA as described herein). In some embodiments, an episome (e.g., an episome comprising one LTR) is produced by homologous recombination (e.g., between viral 5′ and 3′ LTRs, e.g., via strand-invasion or single-strand annealing). In some embodiments, an episome (e.g., an episome comprising on LTR) is produced by ligation of nicks, e.g., present in intermediate products of reverse transcription.

### Introduction of a CAR in T Cells

A LTR retrotransposon-based system described herein may be used to modify immune cells. In some embodiments, a system described herein may be used to modify T cells. In some embodiments, T-cells may include any subpopulation of T-cells, e.g., CD4+, CD8+, gamma-delta, naïve T cells, stem cell memory T cells, central memory T cells, or a mixture of subpopulations. In some embodiments, a system described herein may be used to deliver or modify a T-cell receptor (TCR) in a T cell. In some embodiments, a system described herein may be used to deliver at least one chimeric antigen receptor (CAR) to T-cells. In some embodiments, a system described herein may be used to deliver at least one CAR to natural killer (NK) cells. In some embodiments, a system described herein may be used to deliver at least one CAR to natural killer T (NKT) cells. In some embodiments, a system described herein may be used to deliver at least one CAR to a progenitor cell, e.g., a progenitor cell of T, NK, or NKT cells. In some embodiments, cells modified with at least one CAR (e.g., CAR-T cells, CAR-NK cells, CAR-NKT cells), or a combination of cells modified with at least one CAR (e.g., a mixture of CAR-NK/T cells) are used to treat a condition as identified in the targetable landscape of CAR therapies in MacKay, et al. Nat Biotechnol 38, 233-244 (2020), incorporated by reference herein in its entirety. In some embodiments, the immune cells comprise a CAR specific to a tumor or a pathogen antigen selected from a group consisting of AChR (fetal acetylcholine receptor), ADGRE2, AFP (alpha fetoprotein), BAFF-R, BCMA, CAIX (carbonic anhydrase IX), CCR1, CCR4, CEA (carcinoembryonic antigen), CD3, CD5, CD8, CD7, CD10, CD13, CD14, CD15, CD19, CD20, CD22, CD30, CD33, CLLI, CD34, CD38, CD41, CD44, CD49f, CD56, CD61, CD64, CD68, CD70,CD74, CD99,CD117, CD123, CD133, CD138, CD44v6, CD267, CD269, CDS, CLEC12A, CS1, EGP-2 (epithelial glycoprotein-2), EGP-40 (epithelial glycoprotein-40), EGFR(HER1), EGFR-VIII, EpCAM (epithelial cell adhesion molecule), EphA2, ERBB2 (HER2, human epidermal growth factor receptor 2), ERBB3, ERBB4, FBP (folate-binding protein), Flt3 receptor, folate receptor-a, GD2 (ganglioside G2), GD3 (ganglioside G3), GPC3 (glypican-3), GPI00, hTERT (human telomerase reverse transcriptase), ICAM-1, integrin B7, interleukin 6 receptor, IL13Ra2 (interleukin-13 receptor 30 subunit alpha-2), kappa-light chain, KDR (kinase insert domain receptor), LeY (Lewis Y), L1CAM (LI cell adhesion molecule), LILRB2 (leukocyte immunoglobulin like receptor B2), MARTI, MAGE-A1 (melanoma associated antigen A1), MAGE-A3, MSLN (mesothelin), MUC16 (mucin 16), MUCI (mucin I), KG2D ligands, NY-ESO-1 (cancer-testis antigen), PRI (proteinase 3), TRBCI, TRBC2, TFM-3, TACI, tyrosinase, survivin, hTERT, oncofetal antigen (h5T4), p53, PSCA (prostate stem cell antigen), PSMA (prostate-specific membrane antigen), hRORl, TAG-72 (tumor-associated glycoprotein 72), VEGF-R2 (vascular endothelial growth factor R2), WT-1 (Wilms tumor protein), and antigens of HIV (human immunodeficiency virus), hepatitis B, hepatitis C, CMV (cytomegalovirus), EBV (Epstein-Barr virus), HPV (human papilloma virus).

The LNP formulation C14-4, comprising cholesterol, phospholipid, lipid-anchored PEG, and the ionizable lipid C14-4 (FIG. 2C of Billingsley et al. Nano Lett 20(3):1578-1589 (2020)) can be used for delivery to T cells, such as ex vivo delivery.

Additional edits can be performed on T-cells in order to improve activity of the CAR-T cells against their cognate target. In some embodiments, a second LNP formulation of C14-4 as described comprises a Cas9/gRNA preformed RNP complex, wherein the gRNA targets the Pdcd1 exon 1 for PD-1 inactivation, which can enhance anti-tumor activity of CAR-T cells by disruption of this inhibitory checkpoint that can otherwise trigger suppression of the cells (see Rupp et al. Sci Rep 7:737 (2017)). The application of both nanoparticle formulation thus enables lymphoma targeting by providing the anti-CD19 cargo, while simultaneously boosting efficacy by knocking out the PD-1 checkpoint inhibitor. In some embodiments, cells may be treated with the nanoparticles simultaneously. In some embodiments, the cells may be treated with the nanoparticles in separate steps, e.g., first deliver the RNP for generating the PD-1 knockout, and subsequently treat cells with the nanoparticles carrying the anti-CD19 CAR. In some embodiments, the second component of the system that improves T cell efficacy may result in the knockout of PD-1, TCR, CTLA-4, HLA-I, HLA-II, CSi, CD52, B2M, MHC-I, MHC-II, CD3, FAS, PDC1, CISH, TRAC, or a combination thereof. In some embodiments, knockdown of PD-1, TCR, CTLA-4, HLA-I, HLA-II, CSi, CD52, B2M, MHC-I, MHC-II, CD3, FAS, PDC1, CISH, or TRAC may be preferred, e.g., using siRNA targeting PD-1. In some embodiments, siRNA targeting PD-1 may be achieved using self-delivering RNAi as described by Ligtenberg et al. Mol Ther 26(6):1482-1493 (2018) and in WO2010033247, incorporated herein by reference in its entirety, in which extensive chemical modifications of siRNAs, conferring the resulting hydrophobically modified siRNA molecules the ability to penetrate all cell types ex vivo and in vivo and achieve long-lasting specific target gene knockdown without any additional delivery formulations or techniques. In some embodiments, one or more components of the system may be delivered by other methods, e.g., electroporation. In some embodiments, additional regulators are knocked in to the cells for overexpression to control T cell- and NK cell-mediated immune responses and macrophage engulfment, e.g., PD-L1, HLA-G, CD47 (Han et al. PNAS 116(21):10441-10446 (2019)). Knock-in may be accomplished through application of an additional genome editing system as described herein with a template carrying an expression cassette for one or more such factors (3) with targeting to a safe harbor locus, e.g., AAVS1, e.g., using gRNA GGGGCCACTAGGGACAGGAT (SEQ ID NO: 1) to target the Gene Writer polypeptide to AAVS1.

In order to achieve delivery specifically to T-cells, targeted LNPs (tLNPs) may generated that carry a conjugated mAb against CD4. See, e.g., Ramishetti et al. ACS Nano 9(7):6706-6716 (2015). Alternatively, conjugating a mAb against CD3 can be used to target both CD4+ and CD8⁺ T-cells (Smith et al. Nat Nanotechnol 12(8):813-820 (2017)). In other embodiments, the nanoparticle used to deliver to T-cells in vivo is a constrained nanoparticle that lacks a targeting ligand, as taught by Lokugamage et al. Adv Mater 31(41):e1902251 (2019).

### Retrotransposon Discovery Tools

As the result of repeated mobilization over time, transposable elements in genomic DNA often exist as tandem or interspersed repeats (Jurka Curr Opin Struct Biol 8, 333-337 (1998)). Tools capable of recognizing such repeats can be used to identify new elements from genomic DNA and for populating databases, e.g., Repbase (Jurka et al Cytogenet Genome Res 110, 462-467 (2005)). One such tool for identifying repeats that may comprise transposable elements is RepeatFinder (Volfovsky et al Genome Biol 2 (2001)), which analyzes the repetitive structure of genomic sequences. Repeats can further be collected and analyzed using additional tools, e.g., Censor (Kohany et al BMC Bioinformatics 7, 474 (2006)). The Censor package takes genomic repeats and annotates them using various BLAST approaches against known transposable elements. An all-frames translation can be used to generate the ORF(s) for comparison.

Other exemplary methods for identification of transposable elements include RepeatModeler2, which automates the discovery and annotation of transposable elements in genome sequences (Flynn et al bioRxiv (2019)). In addition to accomplishing this via available packages like Censor, one can perform an all-frames translation of a given genome or sequence and annotate with a protein domain tool like InterProScan, which tags the domains of a given amino acid sequence using the InterPro database (Mitchell et al. Nucleic Acids Res 47, D351-360 (2019)), allowing the identification of potential proteins comprising domains associated with known transposable elements.

In some embodiments, the LTR_STRUC program (e.g., as described by McCarthy et al. 2003, Bioinformatics 19(3): 362-367; incorporated herein by reference in its entirety) can be used to identify LTR retrotransposons suitable for use in the systems, compositions, or methods described herein. In some embodiments, the LTR_FINDER program (e.g., as described by Xu et al. 2007, Nucleic Acids Res. 35(2): W265-W268; incorporated herein by reference in its entirety) can be used to identify LTR retrotransposons suitable for use in the systems, compositions, or methods described herein. In some embodiments, the LTRharvest program (e.g., as described by Ellinghaus et al. 2008, BMC Bioinformatics 9:18; incorporated herein by reference in its entirety) can be used to identify LTR retrotransposons suitable for use in the systems, compositions, or methods described herein. In embodiments, one or more of the following characteristics are used to identify suitable LTR retrotransposons: 1) Elements are generally young based on the nucleotide divergence between the two LTR regions of the retrotransposons; 2) Many LTR elements at different genomic locations share high overall sequence similarity, indicating that they may be the products of recent transposition events; and 3) Target site duplications (TSDs) have been found for most of the complete elements and solo-LTRs. In some instances, retrotransposon integrases create staggered cuts at the target sites, resulting in TSDs as they insert new elements. As such, detection of TSDs flanking genomic retroelement copies can provide evidence for retrotransposition. In certain embodiments, LTR retrotransposons that are active in trans are identified by the presence of copies in the genome that comprise LTRs flanking incomplete gag and pol coding sequences.

Retrotransposons can be further classified according to the reverse transcriptase domain using a tool such as RTclassl (Kapitonov et al Gene 448, 207-213 (2009)).

### Polypeptide Component of Gene Writer Gene Editor System

### RT Domain:

In certain aspects of the present invention, the reverse transcriptase domain of the Gene Writer system is based on a reverse transcriptase domain of an LTR retrotransposon. A wild-type reverse transcriptase domain of an LTR retrotransposon can be used in a Gene Writer system or can be modified (e.g., by insertion, deletion, or substitution of one or more residues) to alter the reverse transcriptase activity for target DNA sequences. In some embodiments the reverse transcriptase is altered from its natural sequence to have altered codon usage, e.g. improved for human cells. In some embodiments the reverse transcriptase domain is a heterologous reverse transcriptase from a different retrovirus, retron, diversity-generating retroelement, retroplasmid, Group II intron, LTR-retrotransposon, non-LTR retrotransposon, or other source, e.g., as exemplified in Table Z1 or as comprising a domain listed in Table Z2 of PCT Application No. PCT/US2021/020943. In certain embodiments, a Gene Writer system includes a polypeptide that comprises a reverse transcriptase domain comprised in Table 10, Table 11, Table X, Table 30, Table 31, or Table 3A or 3B of PCT Application No. PCT/US2021/020943. In embodiments, the amino acid sequence of the reverse transcriptase domain of a Gene Writer system is at least about 50%, at least about 60%, at least about 70%, at least about 80%, at least about 85%, at least about 90%, at least about 95%, at least about 96%, at least about 97%, at least about 98%, at least about 99% identical to the amino acid sequence of a reverse transcriptase domain of a retrotransposon whose DNA sequence is referenced in Table 10, Table 11, Table X, Table Z1, Table Z2, Table 30, Table 31, or Table 3A or 3B of PCT Application No. PCT/US2021/020943. Reverse transcription domains can be identified, for example, based upon homology to other known reverse transcription domains using routine tools as Basic Local Alignment Search Tool (BLAST). In some embodiments, reverse transcriptase domains are modified, for example by site-specific mutation. In some embodiments, the reverse transcriptase domain is engineered to bind a heterologous template RNA. In some embodiments, a polypeptide (e.g., RT domain) comprises an RNA-binding domain, e.g., that specifically binds to an RNA sequence. In some embodiments, a template RNA comprises an RNA sequence that is specifically bound by the RNA-binding domain.

In some embodiments, the RT domain forms a dimer (e.g., a heterodimer or homodimer). In some embodiments, the RT domain is monomeric. In some embodiments, an RT domain, e.g., a retroviral RT domain, naturally functions as a monomer or as a dimer (e.g., heterodimer or homodimer). In some embodiments, an RT domain naturally functions as a monomer, e.g., is derived from a virus wherein it functions as a monomer. Exemplary monomeric RT domains, their viral sources, and the RT signatures associated with them can be found in Table 30 of PCT Application No. PCT/US2021/020943 with descriptions of domain signatures in Table 32. In some embodiments, the RT domain of a system described herein comprises an amino acid sequence of Table 30 in PCT Application No. PCT/US2021/020943, or a functional fragment or variant thereof, or a sequence having at least 70%, 80%, 90%, 95%, or 99% identity thereto. In embodiments, the RT domain is selected from an RT domain from murine leukemia virus (MLV; sometimes referred to as MoMLV) (e.g., P03355), porcine endogenous retrovirus (PERV) (e.g., UniProt Q4VFZ2), mouse mammary tumor virus (MMTV) (e.g., UniProt P03365), Mason-Pfizer monkey virus (MPMV) (e.g., UniProt P07572), bovine leukemia virus (BLV) (e.g., UniProt P03361), human T-cell leukemia virus-1 (HTLV-1) (e.g., UniProt P03362), human foamy virus (HFV) (e.g., UniProt P14350), simian foamy virus (SFV) (e.g., UniProt P23074), or bovine foamy/syncytial virus (BFV/BSV) (e.g., UniProt 041894), or a functional fragment or variant thereof (e.g., an amino acid sequence having at least 70%, 80%, 90%, 95%, or 99% identity thereto). In some embodiments, an RT domain is dimeric in its natural functioning. Exemplary dimeric RT domains, their viral sources, and the RT signatures associated with them can be found in Table 31 of PCT Application No. PCT/US2021/020943 with descriptions of domain signatures in Table 32. In some embodiments, the RT domain of a system described herein comprises an amino acid sequence of Table 31 in PCT Application No. PCT/US2021/020943, or a functional fragment or variant thereof, or a sequence having at least 70%, 80%, 90%, 95%, or 99% identity thereto. In some embodiments, the RT domain is derived from a virus wherein it functions as a dimer. In embodiments, the RT domain is selected from an RT domain from avian sarcoma/leukemia virus (ASLV) (e.g., UniProt AOA142BKH1), Rous sarcoma virus (RSV) (e.g., UniProt P03354), avian myeloblastosis virus (AMV) (e.g., UniProt Q83133), human immunodeficiency virus type I (HIV-1) (e.g., UniProt P03369), human immunodeficiency virus type II (HIV-2) (e.g., UniProt P15833), simian immunodeficiency virus (SIV) (e.g., UniProt P05896), bovine immunodeficiency virus (BIV) (e.g., UniProt P19560), equine infectious anemia virus (EIAV) (e.g., UniProt P03371), or feline immunodeficiency virus (FIV) (e.g., UniProt P16088) (Herschhorn and Hizi Cell Mol Life Sci 67(16):2717-2747 (2010)), or a functional fragment or variant thereof (e.g., an amino acid sequence having at least 70%, 80%, 90%, 95%, or 99% identity thereto). Naturally heterodimeric RT domains may, in some embodiments, also be functional as homodimers. In some embodiments, dimeric RT domains are expressed as fusion proteins, e.g., as homodimeric fusion proteins or heterodimeric fusion proteins. In some embodiments, the RT function of the system is fulfilled by multiple RT domains (e.g., as described herein). In further embodiments, the multiple RT domains are fused or separate, e.g., may be on the same polypeptide or on different polypeptides.

In some embodiments, an RT domain is mutated to increase fidelity compared to an otherwise similar domain without the mutation. For instance, in some embodiments, a YADD (SEQ ID NO: 8) or YMDD (SEQ ID NO: 9) motif in an RT domain (e.g., in a reverse transcriptase) is replaced with YVDD (SEQ ID NO: 10). In embodiments, replacement of the YADD (SEQ ID NO: 8) or YMDD (SEQ ID NO: 9) or YVDD (SEQ ID NO: 10) results in higher fidelity in retroviral reverse transcriptase activity (e.g., as described in Jamburuthugoda and Eickbush J Mol Biol 2011; incorporated herein by reference in its entirety).

The diversity of reverse transcriptases (e.g., comprising RT domains) has been described in, but not limited to, those used by prokaryotes (Zimmerly et al. Microbiol Spectr 3(2):MDNA3-0058-2014 (2015); Lampson B. C. (2007) Prokaryotic Reverse Transcriptases. In: Polaina J., MacCabe A. P. (eds) Industrial Enzymes. Springer, Dordrecht), viruses (Herschhorn et al. Cell Mol Life Sci 67(16):2717-2747 (2010); Menendez-Arias et al. Virus Res 234:153-176 (2017)), and mobile elements (Eickbush et al. Virus Res 134(1-2):221-234 (2008); Craig et al. Mobile DNA II 3rd Ed. DOI:10.1128/9781555819217 (2015)), each of which is incorporated herein by reference.

In some embodiments, a Gene Writing polypeptide comprises the RT domain from a retroviral reverse transcriptase, e.g., a wild-type M-HLV RT, e.g., comprising the following sequence, or a sequence with at least 98% identity thereto:

(SEQ ID NO: 11)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLLI

In some embodiments, a Gene Writing polypeptide comprises the RT domain from a retroviral reverse transcriptase comprising the sequence of amino acids 659-1329 of NP_057933, e.g., as shown below:

(SEQ ID NO: 12)
TLNIEDEHRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGORKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAA

Core RT (bold), annotated per above
RNAseH (underlined), annotated per above

In embodiments, the Gene Writing polypeptide further comprises one additional amino acid at the N-terminus of the sequence of amino acids 659-1329 of NP_057933. In embodiments, the Gene Writing polypeptide further comprises one additional amino acid at the C-terminus of the sequence of amino acids 659-1329 of NP_057933. In embodiments, the Gene Writing polypeptide comprises an RNaseH1 domain (e.g., amino acids 1178-1318 of NP_057933).

In some embodiments, a retroviral reverse transcriptase domain, e.g., M-MLV RT, may comprise one or more mutations from a wild-type sequence that may improve features of the RT, e.g., thermostability, processivity, and/or template binding. In some embodiments, an M-MLV RT domain comprises, relative to the M-MLV (WT) sequence above, one or more mutations, e.g., selected from D200N, L603W, T330P, T306K, W313F, D524G, E562Q, D583N, P51L, S67R, E67K, T197A, H204R, E302K, F309N, L435G, N454K, H594Q, D653N, R110S, K103L, e.g., a combination of mutations, such as D200N, L603W, and T330P, optionally further including T306K and W313F. In some embodiments, an M-MLV RT used herein comprises the mutations D200N, L603W, T330P, T306K and W313F. In embodiments, the mutant M-MLV RT comprises the following amino acid sequence:

(SEQ ID NO: 13)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFN
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGKAGFCRLFIPGFAEMAAPLYPLTKPGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGWLTSEGKEIKNKDEILALLKALFLPKRLSIIHCPGHQKGHSAEARGNR
MADQAARKAAITETPDTSTLLI

### Integrase Domain:

In certain aspects of the present invention, the integrase domain of the Gene Writer system is based on an integrase domain of an LTR retrotransposon. In some embodiments, a Gene Writer polypeptide described herein comprises an integrase domain, e.g., wherein the integrase domain may be part of the RT domain. In some embodiments, an RT domain (e.g., as described herein) comprises an integrase domain. In some embodiments, an RT domain (e.g., as described herein) lacks an integrase domain, or comprises an integrase domain that has been inactivated by mutation or deleted.

In some embodiments, the integrase domain (e.g., a retroviral integrase domain, e.g., a lentiviral integrase domain, e.g., an HIV integrase domain) comprises one or mutations relative to a wild-type equivalent of the integrase domain, wherein the mutated integrase domain has less than 10%, 9%, 8%, 7%, 6%, 5%, 4%, 3%, 2%, or 1% of the activity of the wild-type equivalent of the integrase domain. In certain embodiments, the integrase domain comprises a class I mutation (e.g., as described in Wanisch et al. 2009, Mol. Therap. 17(8): 1316-1332). In certain embodiments, the integrase domain comprises a mutation in a catalytic triad residue (e.g., mutations in 1, 2, or 3 catalytic triad residues). In certain embodiments, the integrase domain comprises a substitution at D64 (e.g., D64V), D116, and/or E152 of the amino acid sequence of an HIV-1 integrase protein. In certain embodiments, the integrase domain comprises a substitution at one or more of the following residues: H12, D64, D116, N120, Q148, F185, W235, R262, R263, K264, K266, and/or K273 of the amino acid sequence of an HIV-1 integrase protein. In certain embodiments, the integrase domain comprises one or more of the following substitutions of the amino acid sequence of an HIV-1 integrase protein: H12A, D64V, D64A, D64E, D116N, N120L, Q148A, F185A, W235E, R262A, R263A, K264H, K264R, K264E, K266R, and/or K273R. In an embodiment, the integrase domain comprises a D64V substitution. In some embodiments, the integrase domain comprises a class II mutation.

In some embodiments, the integrase domain of a Gene Writer system possesses the integration specificity of the native LTR retrotransposon system, e.g., catalyzes integration at the same profile of DNA target sequences. In some embodiments, the integrase domain is modified to have altered DNA target specificity. In some embodiments, the altered DNA target specificity is conferred by mutation or the use of a heterologous integrase domain with a different DNA target sequence preference. In some embodiments, the altered DNA target specificity is conferred by the addition or substitution of a heterologous DNA binding domain in the integrase domain, e.g., a heterologous DNA binding domain as described below.

### DNA Binding Domain:

In certain aspects, the system comprises a DNA-binding domain that is selected, designed, or constructed for binding to a desired host DNA target sequence. In certain embodiments, the DNA-binding domain is a heterologous DNA-binding protein. In some embodiments, the heterologous DNA-binding domain is fused to a domain of a polypeptide of the system, e.g., an integrase domain, to alter the activity of the polypeptide. In some embodiments, the heterologous DNA binding element is a zinc-finger element or a TAL effector element, e.g., a zinc-finger or TAL polypeptide or functional fragment thereof. In some embodiments, the heterologous DNA binding element is a sequence-guided DNA binding element, such as Cas9, Cpf1, or other CRISPR-related protein that has been altered to have no endonuclease activity. In some embodiments the heterologous DNA binding element retains endonuclease activity. In some embodiments, the heterologous DNA-binding domain can be any one or more of Cas9 (e.g., Cas9, Cas9 nickase, dCas9), TAL domain, zinc finger (ZF) domain, Myb domain, combinations thereof, or multiples thereof.

In some embodiments, the DNA binding domain comprises a meganuclease domain (e.g., as described herein, e.g., in the endonuclease domain section), or a functional fragment thereof. In some embodiments, the meganuclease domain possesses endonuclease activity, e.g., double-strand cleavage and/or nickase activity. In other embodiments, the meganuclease domain has reduced activity, e.g., lacks endonuclease activity, e.g., the meganuclease is catalytically inactive. In some embodiments, a catalytically inactive meganuclease is used as a DNA binding domain, e.g., as described in Fonfara et al. Nucleic Acids Res 40(2):847-860 (2012), incorporated herein by reference in its entirety. In embodiments, the DNA binding domain comprises one or more modifications relative to a wild-type DNA binding domain, e.g., a modification via directed evolution, e.g., phage-assisted continuous evolution (PACE).

In certain aspects of the present invention, the host DNA-binding site integrated into by the Gene Writer system can be in a gene, in an intron, in an exon, an ORF, outside of a coding region of any gene, in a regulatory region of a gene, or outside of a regulatory region of a gene. In other aspects, the engineered retrotransposon may bind to one or more than one host DNA sequence. In other aspects, the engineered retrotransposon may have low sequence specificity, e.g., bind to multiple sequences or lack sequence preference.

In some embodiments, a Gene Writing system is used to edit a target locus in multiple alleles. In some embodiments, a Gene Writing system is designed to edit a specific allele. For example, a Gene Writing polypeptide may be directed to a specific sequence that is only present on one allele, but not to a second cognate allele. In some embodiments, a Gene Writing system can alter a haplotype-specific allele. In some embodiments, a Gene Writing system that targets a specific allele preferentially targets that allele, e.g., has at least a 2, 4, 6, 8, or 10-fold preference for a target allele.

### RNase H Domain:

In certain aspects of the present invention, the RNase H domain of the Gene Writer system is based on an RNase H domain of an LTR retrotransposon. In some embodiments, a Gene Writer polypeptide described herein comprises an RNase H domain, e.g., wherein the RNase H domain may be part of the RT domain. In some embodiments, an RT domain (e.g., as described herein) comprises an RNase H domain, e.g., an endogenous RNase H domain or a heterologous RNase H domain. In some embodiments, an RT domain (e.g., as described herein) lacks an RNase H domain. In some embodiments, an RT domain (e.g., as described herein) comprises an RNase H domain that has been added, deleted, mutated, or swapped for a heterologous RNase H domain. In some embodiments, mutation of an RNase H domain yields a polypeptide exhibiting lower RNase activity, e.g., as determined by the methods described in Kotewicz et al. Nucleic Acids Res 16(1):265-277 (1988) (incorporated herein by reference in its entirety), e.g., lower by at least 10%, 20%, 30%, 40%, 50%, 60%, 70%, 80%, or 90% compared to an otherwise similar domain without the mutation. In some embodiments, RNase H activity is abolished.

### Linker Domains:

In some embodiments, a Gene Writer polypeptide may comprise a linker, e.g., a peptide linker, e.g., a linker as described in Table 7. In some embodiments, a Gene Writer polypeptide comprises a flexible linker between the domains, e.g., a linker comprising the amino acid sequence SGGSSGGSSGSETPGTSESATPESSGGSSGGSS (SEQ TD NO: 14).

TABLE 7
Exemplary linker sequences

| | SEQ |
|---|---|
| | ID |
| Amino Acid Sequence | NO: |
| GGS | |
| GGSGGS | 15 |
| GGSGGSGGS | 16 |
| GGSGGSGGSGGS | 17 |
| GGSGGSGGSGGSGGS | 18 |
| GGSGGSGGSGGSGGSGGS | 19 |
| GGGGS | 20 |
| GGGGSGGGGS | 21 |
| GGGGSGGGGSGGGGS | 22 |
| GGGGSGGGGSGGGGSGGGGS | 23 |
| GGGGSGGGGSGGGGSGGGGSGGGGS | 24 |
| GGGGSGGGGSGGGGSGGGGSGGGGSGGGGS | 25 |
| GGG | |
| GGGG | 26 |
| GGGGG | 27 |
| GGGGGG | 28 |
| GGGGGGG | 29 |
| GGGGGGGG | 30 |
| GSS | |
| GSSGSS | 31 |
| GSSGSSGSS | 32 |
| GSSGSSGSSGSS | 33 |
| GSSGSSGSSGSSGSS | 34 |
| GSSGSSGSSGSSGSSGSS | 35 |
| EAAAK | 36 |
| EAAAKEAAAK | 37 |
| EAAAKEAAAKEAAAK | 38 |
| EAAAKEAAAKEAAAKEAAAK | 39 |
| EAAAKEAAAKEAAAKEAAAKEAAAK | 40 |
| EAAAKEAAAKEAAAKEAAAKEAAAKEAAAK | 41 |
| PAP | |
| PAPAP | 42 |
| PAPAPAP | 43 |
| PAPAPAPAP | 44 |
| PAPAPAPAPAP | 45 |
| PAPAPAPAPAPAP | 46 |
| GGSGGG | 47 |
| GGGGGS | 48 |
| GGSGSS | 49 |
| GSSGGS | 50 |
| GGSEAAAK | 51 |
| EAAAKGGS | 52 |
| GGSPAP | 53 |
| PAPGGS | 54 |
| GGGGSS | 55 |
| GSSGGG | 56 |
| GGGEAAAK | 57 |
| EAAAKGGG | 58 |
| GGGPAP | 59 |
| PAPGGG | 60 |
| GSSEAAAK | 61 |
| EAAAKGSS | 62 |
| GSSPAP | 63 |
| PAPGSS | 64 |
| EAAAKPAP | 65 |
| PAPEAAAK | 66 |
| GGSGGGGSS | 67 |
| GGSGSSGGG | 68 |
| GGGGGSGSS | 69 |
| GGGGSSGGS | 70 |
| GSSGGSGGG | 71 |
| GSSGGGGGS | 72 |
| GGSGGGEAAAK | 73 |
| GGSEAAAKGGG | 74 |
| GGGGGSEAAAK | 75 |
| GGGEAAAKGGS | 76 |
| EAAAKGGSGGG | 77 |
| EAAAKGGGGGS | 78 |
| GGSGGGPAP | 79 |
| GGSPAPGGG | 80 |
| GGGGGSPAP | 81 |
| GGGPAPGGS | 82 |
| PAPGGSGGG | 83 |
| PAPGGGGGS | 84 |
| GGSGSSEAAAK | 85 |
| GGSEAAAKGSS | 86 |
| GSSGGSEAAAK | 87 |
| GSSEAAAKGGS | 88 |
| EAAAKGGSGSS | 89 |
| EAAAKGSSGGS | 90 |
| GGSGSSPAP | 91 |
| GGSPAPGSS | 92 |
| GSSGGSPAP | 93 |
| GSSPAPGGS | 94 |
| PAPGGSGSS | 95 |
| PAPGSSGGS | 96 |
| GGSEAAAKPAP | 97 |
| GGSPAPEAAAK | 98 |
| EAAAKGGSPAP | 99 |
| EAAAKPAPGGS | 100 |
| PAPGGSEAAAK | 101 |
| PAPEAAAKGGS | 102 |
| GGGGSSEAAAK | 103 |
| GGGEAAAKGSS | 104 |
| GSSGGGEAAAK | 105 |
| GSSEAAAKGGG | 106 |
| EAAAKGGGGSS | 107 |
| EAAAKGSSGGG | 108 |
| GGGGSSPAP | 109 |
| GGGPAPGSS | 110 |
| GSSGGGPAP | 111 |
| GSSPAPGGG | 112 |
| PAPGGGGSS | 113 |
| PAPGSSGGG | 114 |
| GGGEAAAKPAP | 115 |
| GGGPAPEAAAK | 116 |
| EAAAKGGGPAP | 117 |
| EAAAKPAPGGG | 118 |
| PAPGGGEAAAK | 119 |
| PAPEAAAKGGG | 120 |
| GSSEAAAKPAP | 121 |
| GSSPAPEAAAK | 122 |
| EAAAKGSSPAP | 123 |
| EAAAKPAPGSS | 124 |
| PAPGSSEAAAK | 125 |
| PAPEAAAKGSS | 126 |
| AEAAAKEAAAKEAAAKEAAAKALEAEAAAKEAAAKEAAAKE | 127 |
| AAAKA | |
| GGGGSEAAAKGGGGS | 128 |
| EAAAKGGGGSEAAAK | 129 |
| SGSETPGTSESATPES | 130 |
| GSAGSAAGSGEF | 131 |
| SGGSSGGSSGSETPGTSESATPESSGGSSGGSS | 14 |

### Nucleic Acid Molecules

### Circular RNAs

It is contemplated that it may be useful to employ circular and/or linear RNA states during the formulation, delivery, or Gene Writing reaction within the target cell. Thus, in some embodiments of any of the aspects described herein, a Gene Writing system comprises one or more circular RNAs (circRNAs). In some embodiments of any of the aspects described herein, a Gene Writing system comprises one or more linear RNAs. In some embodiments, a nucleic acid as described herein (e.g., a template nucleic acid, a nucleic acid molecule encoding a Gene Writer polypeptide, or both) is a circRNA. In some embodiments, a circular RNA molecule encodes the Gene Writer polypeptide. In some embodiments, the circRNA molecule encoding the Gene Writer polypeptide is delivered to a host cell. In some embodiments, a circular RNA molecule encodes a recombinase, e.g., as described herein. In some embodiments, the circRNA molecule encoding the recombinase is delivered to a host cell. In some embodiments, the circRNA molecule encoding the Gene Writer polypeptide is linearized (e.g., in the host cell, e.g., in the nucleus of the host cell) prior to translation.

Circular RNAs (circRNAs) have been found to occur naturally in cells and have been found to have diverse functions, including both non-coding and protein coding roles in human cells. It has been shown that a circRNA can be engineered by incorporating a self-splicing intron into an RNA molecule (or DNA encoding the RNA molecule) that results in circularization of the RNA, and that an engineered circRNA can have enhanced protein production and stability (Wesselhoeft et al. Nature Communications 2018). In some embodiments, the Gene Writer™ polypeptide is encoded as circRNA. In certain embodiments, the template nucleic acid is a DNA, such as a dsDNA or ssDNA.

In some embodiments, the circRNA comprises one or more ribozyme sequences. In some embodiments, the ribozyme sequence is activated for autocleavage, e.g., in a host cell, e.g., thereby resulting in linearization of the circRNA. In some embodiments, the ribozyme is activated when the concentration of magnesium reaches a sufficient level for cleavage, e.g., in a host cell. In some embodiments the circRNA is maintained in a low magnesium environment prior to delivery to the host cell. In some embodiments, the ribozyme is a protein-responsive ribozyme. In some embodiments, the ribozyme is a nucleic acid-responsive ribozyme. In some embodiments, the circRNA comprises a cleavage site. In some embodiments, the circRNA comprises a second cleavage site.

In some embodiments, the circRNA is linearized in the nucleus of a target cell. In some embodiments, linearization of a circRNA in the nucleus of a cell involves components present in the nucleus of the cell, e.g., to activate a cleavage event. For example, the B2 and ALU retrotransposons contain self-cleaving ribozymes whose activity is enhanced by interaction with the Polycomb protein, EZH2 (Hernandez et al. PNAS 117(1):415-425 (2020)). Thus, in some embodiments, a ribozyme, e.g., a ribozyme from a B2 or ALU element, that is responsive to a nuclear element, e.g., a nuclear protein, e.g., a genome-interacting protein, e.g., an epigenetic modifier, e.g., EZH2, is incorporated into a circRNA, e.g., of a Gene Writing system. In some embodiments, nuclear localization of the circRNA results in an increase in autocatalytic activity of the ribozyme and linearization of the circRNA.

In some embodiments, the ribozyme is heterologous to one or more of the other components of the Gene Writing system. In some embodiments, an inducible ribozyme (e.g., in a circRNA as described herein) is created synthetically, for example, by utilizing a protein ligand-responsive aptamer design. A system for utilizing the satellite RNA of tobacco ringspot virus hammerhead ribozyme with an MS2 coat protein aptamer has been described (Kennedy et al. Nucleic Acids Res 42(19):12306-12321 (2014), incorporated herein by reference in its entirety) that results in activation of the ribozyme activity in the presence of the MS2 coat protein. In embodiments, such a system responds to protein ligand localized to the cytoplasm or the nucleus. In some embodiments the protein ligand is not MS2. Methods for generating RNA aptamers to target ligands have been described, for example, based on the systematic evolution of ligands by exponential enrichment (SELEX) (Tuerk and Gold, Science 249(4968):505-510 (1990); Ellington and Szostak, Nature 346(6287):818-822 (1990); the methods of each of which are incorporated herein by reference) and have, in some instances, been aided by in silico design (Bell et al. PNAS 117(15):8486-8493, the methods of which are incorporated herein by reference). Thus, in some embodiments, an aptamer for a target ligand is generated and incorporated into a synthetic ribozyme system, e.g., to trigger ribozyme-mediated cleavage and circRNA linearization, e.g., in the presence of the protein ligand. In some embodiments, circRNA linearization is triggered in the cytoplasm, e.g., using an aptamer that associates with a ligand in the cytoplasm. In some embodiments, circRNA linearization is triggered in the nucleus, e.g., using an aptamer that associates with a ligand in the nucleus. In embodiments, the ligand in the nucleus comprises an epigenetic modifier or a transcription factor. In some embodiments the ligand that triggers linearization is present at higher levels in on-target cells than off-target cells.

It is further contemplated that a nucleic acid-responsive ribozyme system can be employed for circRNA linearization. For example, biosensors that sense defined target nucleic acid molecules to trigger ribozyme activation are described, e.g., in Penchovsky (Biotechnology Advances 32(5):1015-1027 (2014), incorporated herein by reference). By these methods, a ribozyme naturally folds into an inactive state and is only activated in the presence of a defined target nucleic acid molecule (e.g., an RNA molecule). In some embodiments, a circRNA of a Gene Writing system comprises a nucleic acid-responsive ribozyme that is activated in the presence of a defined target nucleic acid, e.g., an RNA, e.g., an mRNA, miRNA, guide RNA, gRNA, sgRNA, ncRNA, lncRNA, tRNA, snRNA, or mtRNA. In some embodiments the nucleic acid that triggers linearization is present at higher levels in on-target cells than off-target cells.

In some embodiments of any of the aspects herein, a Gene Writing system incorporates one or more ribozymes with inducible specificity to a target tissue or target cell of interest, e.g., a ribozyme that is activated by a ligand or nucleic acid present at higher levels in a target tissue or target cell of interest. In some embodiments, the Gene Writing system incorporates a ribozyme with inducible specificity to a subcellular compartment, e.g., the nucleus, nucleolus, cytoplasm, or mitochondria. In some embodiments, the ribozyme that is activated by a ligand or nucleic acid present at higher levels in the target subcellular compartment. In some embodiments, an RNA component of a Gene Writing system is provided as circRNA, e.g., that is activated by linearization. In some embodiments, linearization of a circRNA encoding a Gene Writing polypeptide activates the molecule for translation. In some embodiments, a signal that activates a circRNA component of a Gene Writing system is present at higher levels in on-target cells or tissues, e.g., such that the system is specifically activated in these cells.

In some embodiments, an RNA component of a Gene Writing system is provided as a circRNA that is inactivated by linearization. In some embodiments, a circRNA encoding the Gene Writer polypeptide is inactivated by cleavage and degradation. In some embodiments, a circRNA encoding the Gene Writing polypeptide is inactivated by cleavage that separates a translation signal from the coding sequence of the polypeptide. In some embodiments, a signal that inactivates a circRNA component of a Gene Writing system is present at higher levels in off-target cells or tissues, such that the system is specifically inactivated in these cells.

### Doggybone DNA

In some embodiments, nucleic acid (e.g., encoding a polypeptide, or a template DNA, or both) delivered to cells is covalently closed linear DNA, or so-called “doggybone” DNA. During its lifecycle, the bacteriophage N15 employs protelomerase to convert its genome from circular plasmid DNA to a linear plasmid DNA (Ravin et al. J Mol Biol 2001). This process has been adapted for the production of covalently closed linear DNA in vitro (see, for example, WO2010086626A1). In some embodiments, a protelomerase is contacted with a DNA containing one or more protelomerase recognition sites, wherein protelomerase results in a cut at the one or more sites and subsequent ligation of the complementary strands of DNA, resulting in the covalent linkage between the complementary strands. In some embodiments, nucleic acid (e.g., encoding a transposase, or a template DNA, or both) is first generated as circular plasmid DNA containing a single protelomerase recognition site that is then contacted with protelomerase to yield a covalently closed linear DNA. In some embodiments, nucleic acid (e.g., encoding a transposase, or a template DNA, or both) flanked by protelomerase recognition sites on plasmid or linear DNA is contacted with protelomerase to generate a covalently closed linear DNA containing only the DNA contained between the protelomerase recognition sites. In some embodiments, the approach of flanking the desired nucleic acid sequence by protelomerase recognition sites results in covalently closed circular DNA lacking plasmid elements used for bacterial cloning and maintenance. In some embodiments, the plasmid or linear DNA containing the nucleic acid and one or more protelomerase recognition sites is optionally amplified prior to the protelomerase reaction, e.g., by rolling circle amplification or PCR.

### Chemically Modified Nucleic Acids and Nucleic Acid End Features:

A nucleic acid described herein (e.g., a template nucleic acid, e.g., a template RNA; or a nucleic acid (e.g., mRNA) encoding a GeneWriter) can comprise unmodified or modified nucleobases. Naturally occurring RNAs are synthesized from four basic ribonucleotides: ATP, CTP, UTP and GTP, but may contain post-transcriptionally modified nucleotides. Further, approximately one hundred different nucleoside modifications have been identified in RNA (Rozenski, J, Crain, P, and McCloskey, J. (1999). The RNA Modification Database: 1999 update. Nucl Acids Res 27:196-197). An RNA can also comprise wholly synthetic nucleotides that do not occur in nature.

In some embodiments, the chemically modification is one provided in PCT/US2016/032454, US Pat. Pub. No. 20090286852, of International Application No. WO/2012/019168, WO/2012/045075, WO/2012/135805, WO/2012/158736, WO/2013/039857, WO/2013/039861, WO/2013/052523, WO/2013/090648, WO/2013/096709, WO/2013/101690, WO/2013/106496, WO/2013/130161, WO/2013/151669, WO/2013/151736, WO/2013/151672, WO/2013/151664, WO/2013/151665, WO/2013/151668, WO/2013/151671, WO/2013/151667, WO/2013/151670, WO/2013/151666, WO/2013/151663, WO/2014/028429, WO/2014/081507, WO/2014/093924, WO/2014/093574, WO/2014/113089, WO/2014/144711, WO/2014/144767, WO/2014/144039, WO/2014/152540, WO/2014/152030, WO/2014/152031, WO/2014/152027, WO/2014/152211, WO/2014/158795, WO/2014/159813, WO/2014/164253, WO/2015/006747, WO/2015/034928, WO/2015/034925, WO/2015/038892, WO/2015/048744, WO/2015/051214, WO/2015/051173, WO/2015/051169, WO/2015/058069, WO/2015/085318, WO/2015/089511, WO/2015/105926, WO/2015/164674, WO/2015/196130, WO/2015/196128, WO/2015/196118, WO/2016/011226, WO/2016/011222, WO/2016/011306, WO/2016/014846, WO/2016/022914, WO/2016/036902, WO/2016/077125, or WO/2016/077123, each of which is herein incorporated by reference in its entirety. It is understood that incorporation of a chemically modified nucleotide into a polynucleotide can result in the modification being incorporated into a nucleobase, the backbone, or both, depending on the location of the modification in the nucleotide. In some embodiments, the backbone modification is one provided in EP 2813570, which is herein incorporated by reference in its entirety. In some embodiments, the modified cap is one provided in US Pat. Pub. No. 20050287539, which is herein incorporated by reference in its entirety.

In some embodiments, the chemically modified nucleic acid (e.g., RNA, e.g., mRNA) comprises one or more of ARCA: anti-reverse cap analog (m27.3′-OGP3G), GP3G (Unmethylated Cap Analog), m7GP3G (Monomethylated Cap Analog), m32.2.7GP3G (Trimethylated Cap Analog), m5CTP (5′-methyl-cytidine triphosphate), m6ATP (N6-methyl-adenosine-5′-triphosphate), s2UTP (2-thio-uridine triphosphate), and Ψ (pseudouridine triphosphate).

In some embodiments, the chemically modified nucleic acid comprises a 5′ cap, e.g.: a 7-methylguanosine cap (e.g., a 0-Me-m7G cap); a hypermethylated cap analog; an NAD+-derived cap analog (e.g., as described in Kiledjian, Trends in Cell Biology 28, 454-464 (2018)); or a modified, e.g., biotinylated, cap analog (e.g., as described in Bednarek et al., Phil Trans R Soc B 373, 20180167 (2018)).

In some embodiments, the chemically modified nucleic acid comprises a 3′ feature selected from one or more of: a polyA tail; a 16-nucleotide long stem-loop structure flanked by unpaired 5 nucleotides (e.g., as described by Mannironi et al., Nucleic Acid Research 17, 9113-9126 (1989)); a triple-helical structure (e.g., as described by Brown et al., PNAS 109, 19202-19207 (2012)); a tRNA, Y RNA, or vault RNA structure (e.g., as described by Labno et al., Biochemica et Biophysica Acta 1863, 3125-3147 (2016)); incorporation of one or more deoxyribonucleotide triphosphates (dNTPs), 2′O-Methylated NTPs, or phosphorothioate-NTPs; a single nucleotide chemical modification (e.g., oxidation of the 3′ terminal ribose to a reactive aldehyde followed by conjugation of the aldehyde-reactive modified nucleotide); or chemical ligation to another nucleic acid molecule.

In some embodiments, the nucleic acid (e.g., template nucleic acid) comprises one or more modified nucleotides, e.g., selected from dihydrouridine, inosine, 7-methylguanosine, 5-methylcytidine (5mC), 5′ Phosphate ribothymidine, 2′-O-methyl ribothymidine, 2′-O-ethyl ribothymidine, 2′-fluoro ribothymidine, C-5 propynyl-deoxycytidine (pdC), C-5 propynyl-deoxyuridine (pdU), C-5 propynyl-cytidine (pC), C-5 propynyl-uridine (pU), 5-methyl cytidine, 5-methyl uridine, 5-methyl deoxycytidine, 5-methyl deoxyuridine methoxy, 2,6-diaminopurine, 5′-Dimethoxytrityl-N4-ethyl-2′-deoxycytidine, C-5 propynyl-f-cytidine (pfC), C-5 propynyl-f-uridine (pfU), 5-methyl f-cytidine, 5-methyl f-uridine, C-5 propynyl-m-cytidine (pmC), C-5 propynyl-f-uridine (pmU), 5-methyl m-cytidine, 5-methyl m-uridine, LNA (locked nucleic acid), MGB (minor groove binder) pseudouridine (Ψ), 1-N-methylpseudouridine (1-Me-Ψ), or 5-methoxyuridine (5-MO-U).

In some embodiments, the nucleic acid comprises a backbone modification, e.g., a modification to a sugar or phosphate group in the backbone. In some embodiments, the nucleic acid comprises a nucleobase modification.

In some embodiments, the nucleic acid comprises one or more chemically modified nucleotides of Table M1, one or more chemical backbone modifications of Table M2, one or more chemically modified caps of Table M3. For instance, in some embodiments, the nucleic acid comprises two or more (e.g., 3, 4, 5, 6, 7, 8, 9, or 10 or more) different types of chemical modifications. As an example, the nucleic acid may comprise two or more (e.g., 3, 4, 5, 6, 7, 8, 9, or 10 or more) different types of modified nucleobases, e.g., as described herein, e.g., in Table M1. Alternatively or in combination, the nucleic acid may comprise two or more (e.g., 3, 4, 5, 6, 7, 8, 9, or 10 or more) different types of backbone modifications, e.g., as described herein, e.g., in Table M2. Alternatively or in combination, the nucleic acid may comprise one or more modified cap, e.g., as described herein, e.g., in Table M3. For instance, in some embodiments, the nucleic acid comprises one or more type of modified nucleobase and one or more type of backbone modification; one or more type of modified nucleobase and one or more modified cap; one or more type of modified cap and one or more type of backbone modification; or one or more type of modified nucleobase, one or more type of backbone modification, and one or more type of modified cap.

In some embodiments, the nucleic acid comprises one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) modified nucleobases. In some embodiments, all nucleobases of the nucleic acid are modified. In some embodiments, the nucleic acid is modified at one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) positions in the backbone. In some embodiments, all backbone positions of the nucleic acid are modified.

TABLE M1
Modified nucleotides

| | |
|---|---|
| 5-aza-uridine | N2-methyl-6-thio-guanosine |
| 2-thio-5-aza-midine | N2,N2-dimethyl-6-thio-guanosine |
| 2-thiouridine | pyridin-4-one ribonucleoside |
| 4-thio-pseudouridine | 2-thio-5-aza-uridine |
| 2-thio-pseudouridine | 2-thiomidine |
| 5-hydroxyuridine | 4-thio-pseudomidine |
| 3-methyluridine | 2-thio-pseudowidine |
| 5-carboxymethyl-uridine | 3-methylmidine |
| 1-carboxymethyl-pseudouridine | 1-propynyl-pseudomidine |
| 5-propynyl-uridine | 1-methyl-1-deaza-pseudomidine |
| 1-propynyl-pseudouridine | 2-thio-1-methyl-1-deaza-pseudouridine |
| 5-taurinomethyluridine | 4-methoxy-pseudomidine |
| 1-taurinomethyl-pseudouridine | 5′-O-(1-Thiophosphate)-Adenosine |
| 5-taurinomethyl-2-thio-uridine | 5′-O-(1-Thiophosphate)-Cytidine |
| 1-taurinomethyl-4-thio-uridine | 5′-O-(1-thiophosphate)-Guanosine |
| 5-methyl-uridine | 5′-O-(1-Thiophophate)-Uridine |
| 1-methyl-pseudouridine | 5′-O-(1-Thiophosphate)-Pseudouridine |
| 4-thio-1-methyl-pseudouridine | 2′-O-methyl-Adenosine |
| 2-thio-1-methyl-pseudouridine | 2′-O-methyl-Cytidine |
| 1-methyl-1-deaza-pseudouridine | 2′-O-methyl-Guanosine |
| 2-thio-1-methyl-1-deaza-pseudomidine | 2′-O-methyl-Uridine |
| dihydrouridine | 2′-O-methyl-Pseudouridine |
| dihydropseudouridine | 2′-O-methyl-Inosine |
| 2-thio-dihydromidine | 2-methyladenosine |
| 2-thio-dihydropseudouridine | 2-methylthio-N6-methyladenosine |
| 2-methoxyuridine | 2-methylthio-N6 isopentenyladenosine |
| 2-methoxy-4-thio-uridine | 2-methylthio-N6-(cis- |
| 4-methoxy-pseudouridine | hydroxyisopentenyl)adenosine |
| 4-methoxy-2-thio-pseudouridine | N6-methyl-N6-threonylcarbamoyladenosine |
| 5-aza-cytidine | N6-hydroxynorvalylcarbamoyladenosine |
| pseudoisocytidine | 2-methylthio-N6-hydroxynorvalyl |
| 3-methyl-cytidine | carbamoyladenosine |
| N4-acetylcytidine | 2′-O-ribosyladenosine (phosphate) |
| 5-formylcytidine | 1,2′-O-dimethylinosine |
| N4-methylcytidine | 5,2′-O-dimethylcytidine |
| 5-hydroxymethylcytidine | N4-acetyl-2′-O-methylcytidine |
| 1-methyl-pseudoisocytidine | Lysidine |
| pyrrolo-cytidine | 7-methylguanosine |
| pyrrolo-pseudoisocytidine | N2,2′-O-dimethylguanosine |
| 2-thio-cytidine | N2,N2,2′-O-trimethylguanosine |
| 2-thio-5-methyl-cytidine | 2′-O-ribosylguanosine (phosphate) |
| 4-thio-pseudoisocytidine | Wybutosine |
| 4-thio-1-methyl-pseudoisocytidine | Peroxywybutosine |
| 4-thio-1-methyl-1-deaza-pseudoisocytidine | Hydroxywybutosine |
| 1-methyl-1-deaza-pseudoisocytidine | undermodified hydroxywybutosine |
| zebularine | methylwyosine |
| 5-aza-zebularine | queuosine |
| 5-methyl-zebularine | epoxyqueuosine |
| 5-aza-2-thio-zebularine | galactosyl-queuosine |
| 2-thio-zebularine | mannosyl-queuosine |
| 2-methoxy-cytidine | 7-cyano-7-deazaguanosine |
| 2-methoxy-5-methyl-cytidine | 7-aminomethyl-7-deazaguanosine |
| 4-methoxy-pseudoisocytidine | archaeosine |
| 4-methoxy-1-methyl-pseudoisocytidine | 5,2′-O-dimethyluridine |
| 2-aminopurine | 4-thiouridine |
| 2,6-diaminopurine | 5-methyl-2-thiouridine |
| 7-deaza-adenine | 2-thio-2′-O-methyluridine |
| 7-deaza-8-aza-adenine | 3-(3-amino-3-carboxypropyl)uridine |
| 7-deaza-2-aminopurine | 5-methoxyuridine |
| 7-deaza-8-aza-2-aminopurine | uridine 5-oxyacetic acid |
| 7-deaza-2,6-diaminopurine | uridine 5-oxyacetic acid methyl ester |
| 7-deaza-8-aza-2,6-diaminopurine | 5-(carboxyhydroxymethyl)uridine) |
| 1-methyladenosine | 5-(carboxyhydroxymethyl)uridine methyl ester |
| N6-isopentenyladenosine | 5-methoxycarbonylmethyluridine |
| N6-(cis-hydroxyisopentenyl)adenosine | 5-methoxycarbonylmethyl-2′-O-methyluridine |
| 2-methylthio-N6-(cis-hydroxyisopentenyl) | 5-methoxycarbonylmethyl-2-thiouridine |
| adenosine | 5-aminomethyl-2-thiouridine |
| N6-glycinylcarbamoyladenosine | 5-methylaminomethyluridine |
| N6-threonylcarbamoyladenosine | 5-methylaminomethyl-2-thiouridine |
| 2-methylthio-N6-threonyl carbamoyladenosine | 5-methylaminomethyl-2-selenouridine |
| N6,N6-dimethyladenosine | 5-carbamoylmethyluridine |
| 7-methyladenine | 5-carbamoylmethyl-2′-O-methyluridine |
| 2-methylthio-adenine | 5-carboxymethylaminomethyluridine |
| 2-methoxy-adenine | 5-carboxymethylaminomethyl-2′-O-methyluridine |
| inosine | 5-carboxymethylaminomethyl-2-thiouridine |
| 1-methyl-inosine | N4,2′-O-dimethylcytidine |
| wyosine | 5-carboxymethyluridine |
| wybutosine | N6,2′-O-dimethyladenosine |
| 7-deaza-guanosine | N,N6,O-2′-trimethyladenosine |
| 7-deaza-8-aza-guanosine | N2,7-dimethylguanosine |
| 6-thio-guanosine | N2,N2,7-trimethylguanosine |
| 6-thio-7-deaza-guanosine | 3,2′-O-dimethyluridine |
| 6-thio-7-deaza-8-aza-guanosine | 5-methyldihydrouridine |
| 7-methyl-guanosine | 5-formyl-2′-O-methylcytidine |
| 6-thio-7-methyl-guanosine | 1,2′-O-dimethylguanosine |
| 7-methylinosine | 4-demethylwyosine |
| 6-methoxy-guanosine | Isowyosine |
| 1-methylguanosine | N6-acetyladenosine |
| N2-methylguanosine | |
| N2,N2-dimethylguanosine | |
| 8-oxo-guanosine | |
| 7-methyl-8-oxo-guanosine | |
| 1-methyl-6-thio-guanosine | |

TABLE M2
Backbone modifications

2′-O-Methyl backbone
Peptide Nucleic Acid (PNA) backbone
phosphorothioate backbone
morpholino backbone
carbamate backbone
siloxane backbone
sulfide backbone
sulfoxide backbone
sulfone backbone
formacetyl backbone
thioformacetyl backbone
methyleneformacetyl backbone
riboacetyl backbone
alkene containing backbone
sulfamate backbone
sulfonate backbone
sulfonamide backbone
methyleneimino backbone
methylenehydrazino backbone
amide backbone

TABLE M3
Modified caps

m7GpppA
m7GpppC
m2,7GpppG
m2,2,7GpppG
m7Gpppm7G
m7,2′OmeGpppG
m72′dGpppG
m7,3′OmeGpppG
m7,3′dGpppG
GppppG
m7GppppG
m7GppppA
m7GppppC
m2,7GppppG
m2,2,7GppppG
m7Gppppm7G
m7,2OmeGppppG
m72′dGppppG
m7,3′OmeGppppG
m7,3′dGppppG

### Production of Compositions and Systems

Methods of designing and constructing nucleic acid constructs and proteins or polypeptides (such as the systems, constructs and polypeptides described herein) are known. Generally, recombinant methods may be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012).

The disclosure provides, in part, a nucleic acid, e.g., vector, encoding a Gene Writer polypeptide described herein, a template nucleic acid described herein, or both. In some embodiments, a vector comprises a selective marker, e.g., an antibiotic resistance marker. In some embodiments, the antibiotic resistance marker is a kanamycin resistance marker. In some embodiments, the antibiotic resistance marker does not confer resistance to beta-lactam antibiotics. In some embodiments, the vector does not comprise an ampicillin resistance marker. In some embodiments, the vector comprises a kanamycin resistance marker and does not comprise an ampicillin resistance marker. In some embodiments, a vector encoding a Gene Writer polypeptide is integrated into a target cell genome (e.g., upon administration to a target cell, tissue, organ, or subject). In some embodiments, a vector encoding a Gene Writer polypeptide is not integrated into a target cell genome (e.g., upon administration to a target cell, tissue, organ, or subject). In some embodiments, a vector encoding a template nucleic acid (e.g., template RNA) is not integrated into a target cell genome (e.g., upon administration to a target cell, tissue, organ, or subject). In some embodiments, if a vector is integrated into a target site in a target cell genome, the selective marker is not integrated into the genome. In some embodiments, if a vector is integrated into a target site in a target cell genome, genes or sequences involved in vector maintenance (e.g., plasmid maintenance genes) are not integrated into the genome. In some embodiments, if a vector is integrated into a target site in a target cell genome, transfer regulating sequences (e.g., inverted terminal repeats, e.g., from an AAV) are not integrated into the genome. In some embodiments, administration of a vector (e.g., encoding a Gene Writer polypeptide described herein, a template nucleic acid described herein, or both) to a target cell, tissue, organ, or subject results in integration of a portion of the vector into one or more target sites in the genome(s) of said target cell, tissue, organ, or subject. In some embodiments, less than 99, 95, 90, 80, 70, 60, 50, 40, 30, 20, 10, 5, 4, 3, 2, or 1% of target sites (e.g., no target sites) comprising integrated material comprise a selective marker (e.g., an antibiotic resistance gene), a transfer regulating sequence (e.g., an inverted terminal repeat, e.g., from an AAV), or both from the vector.

Exemplary methods for producing a therapeutic pharmaceutical protein or polypeptide described herein involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture recombinant protein. Examples of mammalian expression systems include CHO, COS, HEK293, HeLA, and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

### Production of RNA Components:

Further included here are compositions and methods for the assembly of full or partial template RNA molecules. In some embodiments, RNA molecules may be assembled by the connection of two or more (e.g., two, three, four, five, six, seven, eight, nine, ten, or more) RNA segments with each other. In an aspect, the disclosure provides methods for producing nucleic acid molecules, the methods comprising contacting two or more linear RNA segments with each other under conditions that allow for the 5′ terminus of a first RNA segment to be covalently linked with the 3′ terminus of a second RNA segment. In some embodiments, the joined molecule may be contacted with a third RNA segment under conditions that allow for the 5′ terminus of the joined molecule to be covalently linked with the 3′ terminus of the third RNA segment. In embodiments, the method further comprises joining a fourth, fifth, or additional RNA segments to the elongated molecule. This form of assembly may, in some instances, allow for rapid and efficient assembly of RNA molecules.

In some embodiments, RNA segments may be produced by chemical synthesis. In some embodiments, RNA segments may be produced by in vitro transcription of a nucleic acid template, e.g., by providing an RNA polymerase to act on a cognate promoter of a DNA template to produce an RNA transcript. In some embodiments, in vitro transcription is performed using, e.g., a T7, T3, or SP6 RNA polymerase, or a derivative thereof, acting on a DNA, e.g., dsDNA, ssDNA, linear DNA, plasmid DNA, linear DNA amplicon, linearized plasmid DNA, e.g., encoding the RNA segment, e.g., under transcriptional control of a cognate promoter, e.g., a T7, T3, or SP6 promoter. In some embodiments, a combination of chemical synthesis and in vitro transcription is used to generate the RNA segments for assembly. In embodiments, the gRNA, upstream target homology, and Gene Writer polypeptide binding segments are produced by chemical synthesis and the heterologous object sequence segment is produced by in vitro transcription. Without wishing to be bound by theory, in vitro transcription may be better suited for the production of longer RNA molecules. In some embodiments, reaction temperature for in vitro transcription may be lowered, e.g., be less than 37° C. (e.g., between 0-10 C, 10-20 C, or 20-30 C), to result in a higher proportion of full-length transcripts (Krieg Nucleic Acids Res 18:6463 (1990)). In some embodiments, a protocol for improved synthesis of long transcripts is employed to synthesize a long template RNA, e.g., a template RNA greater than 5 kb, such as the use of e.g., T7 RiboMAX Express, which can generate 27 kb transcripts in vitro (Thiel et al. J Gen Virol 82(6):1273-1281 (2001)). In some embodiments, modifications to RNA molecules as described herein may be incorporated during synthesis of RNA segments (e.g., through the inclusion of modified nucleotides or alternative binding chemistries), following synthesis of RNA segments through chemical or enzymatic processes, following assembly of one or more RNA segments, or a combination thereof.

In some embodiments, an mRNA of the system (e.g., an mRNA encoding a Gene Writer polypeptide) is synthesized in vitro using T7 polymerase-mediated DNA-dependent RNA transcription from a linearized DNA template, where UTP is optionally substituted with 1-methylpseudoUTP. In some embodiments, the transcript incorporates 5′ and 3′ UTRs, e.g., GGGAAAUAAGAGAGAAAAGAAGAGUAAGAAGAAAUAUAAGAGCCACC (SEQ ID NO: 132) and UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 133), or functional fragments or variants thereof, and optionally includes a poly-A tail, which can be encoded in the DNA template or added enzymatically following transcription. In some embodiments, a donor methyl group, e.g., S-adenosylmethionine, is added to a methylated capped RNA with cap 0 structure to yield a cap 1 structure that increases mRNA translation efficiency (Richner et al. Cell 168(6): P1114-1125 (2017)).

In some embodiments, the transcript from a T7 promoter starts with a GGG motif. In some embodiments, a transcript from a T7 promoter does not start with a GGG motif. It has been shown that a GGG motif at the transcriptional start, despite providing superior yield, may lead to T7 RNAP synthesizing a ladder of poly(G) products as a result of slippage of the transcript on the three C residues in the template strand from +1 to +3 (Imburgio et al. Biochemistry 39(34):10419-10430 (2000). For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

In some embodiments, RNA segments may be connected to each other by covalent coupling. In some embodiments, an RNA ligase, e.g., T4 RNA ligase, may be used to connect two or more RNA segments to each other. When a reagent such as an RNA ligase is used, a 5′ terminus is typically linked to a 3′ terminus. In some embodiments, if two segments are connected, then there are two possible linear constructs that can be formed (i.e., (1) 5′-Segment 1-Segment 2-3′ and (2) 5′-Segment 2-Segment 1-3′). In some embodiments, intramolecular circularization can also occur. Both of these issues can be addressed, for example, by blocking one 5′ terminus or one 3′ terminus so that RNA ligase cannot ligate the terminus to another terminus. In embodiments, if a construct of 5′-Segment 1-Segment 2-3′ is desired, then placing a blocking group on either the 5′ end of Segment 1 or the 3′ end of Segment 2 may result in the formation of only the correct linear ligation product and/or prevent intramolecular circularization. Compositions and methods for the covalent connection of two nucleic acid (e.g., RNA) segments are disclosed, for example, in US20160102322A1 (incorporated herein by reference in its entirety), along with methods including the use of an RNA ligase to directionally ligate two single-stranded RNA segments to each other.

One example of an end blocker that may be used in conjunction with, for example, T4 RNA ligase, is a dideoxy terminator. T4 RNA ligase typically catalyzes the ATP-dependent ligation of phosphodiester bonds between 5′-phosphate and 3′-hydroxyl termini. In some embodiments, when T4 RNA ligase is used, suitable termini must be present on the termini being ligated. One means for blocking T4 RNA ligase on a terminus comprises failing to have the correct terminus format. Generally, termini of RNA segments with a 5-hydroxyl or a 3′-phosphate will not act as substrates for T4 RNA ligase.

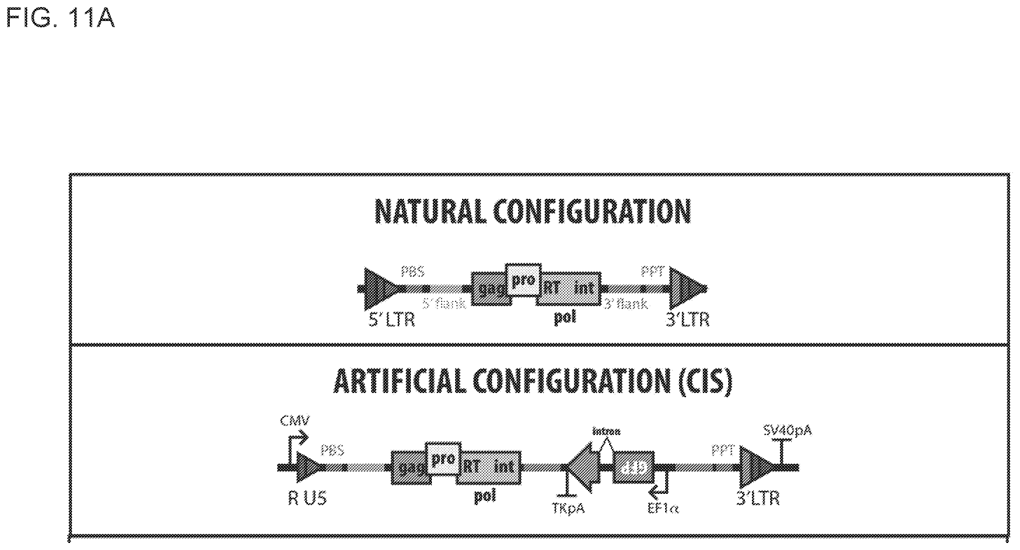

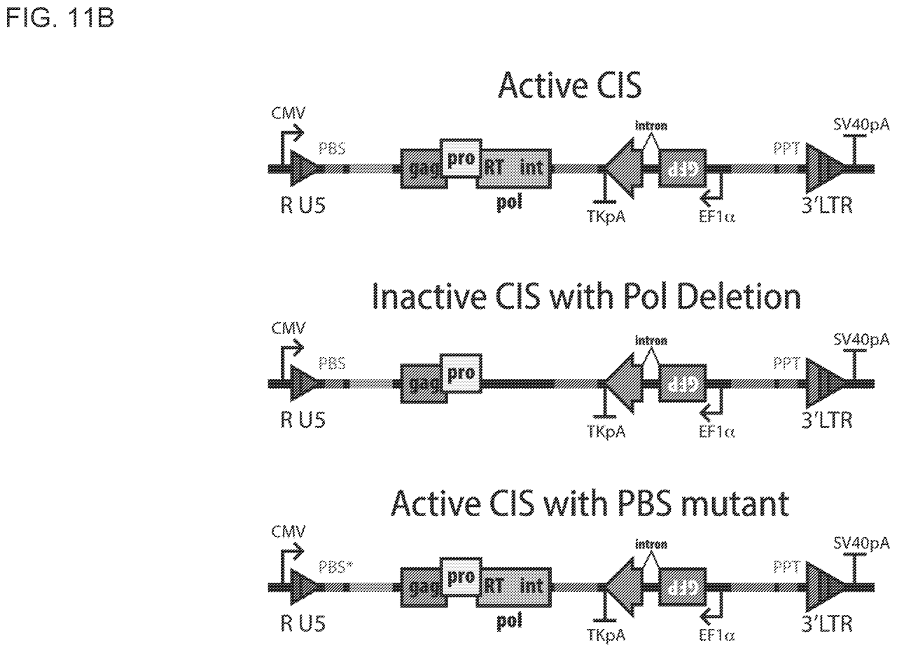

Additional exemplary methods that may be used to connect RNA segments is by click chemistry (e.g., as described in U.S. Pat. Nos. 7,375,234 and 7,070,941, and US Patent Publication No. 2013/0046084, the entire disclosures of which are incorporated herein by reference). For example, one exemplary click chemistry reaction is between an alkyne group and an azide group (see FIG. 11 of US20160102322A1, which is incorporated herein by reference in its entirety). Any click reaction may potentially be used to link RNA segments (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). In some embodiments, ligation of RNA molecules using a click chemistry reaction is advantageous because click chemistry reactions are fast, modular, efficient, often do not produce toxic waste products, can be done with water as a solvent, and/or can be set up to be stereospecific.

In some embodiments, RNA segments may be connected using an Azide-Alkyne Huisgen Cycloaddition. reaction, which is typically a 1,3-dipolar cycloaddition between an azide and a terminal or internal alkyne to give a 1,2,3-triazole for the ligation of RNA segments. Without wishing to be bound by theory, one advantage of this ligation method may be that this reaction can initiated by the addition of required Cu(I) ions. Other exemplary mechanisms by which RNA segments may be connected include, without limitatoin, the use of halogens (F, Br—, I—)/alkynes addition reactions, carbonyls/sulfhydryls/maleimide, and carboxyl/amine linkages. For example, one RNA molecule may be modified with thiol at 3′ (using disulfide amidite and universal support or disulfide modified support), and the other RNA molecule may be modified with acrydite at 5′ (using acrylic phosphoramidite), then the two RNA molecules can be connected by a Michael addition reaction. This strategy can also be applied to connecting multiple RNA molecules stepwise. Also provided are methods for linking more than two (e.g., three, four, five, six, etc.) RNA molecules to each other. Without wishing to be bound by theory, this may be useful when a desired RNA molecule is longer than about 40 nucleotides, e.g., such that chemical synthesis efficiency degrades, e.g., as noted in US20160102322A1 (incorporated herein by reference in its entirety).

### Kits, Articles of Manufacture, and Pharmaceutical Compositions:

In an aspect the disclosure provides a kit comprising a Gene Writer or a Gene Writing system, e.g., as described herein. In some embodiments, the kit comprises a Gene Writer polypeptide (or a nucleic acid encoding the polypeptide) and a template RNA (or DNA encoding the template RNA). In some embodiments, the kit further comprises a reagent for introducing the system into a cell, e.g., transfection reagent, LNP, and the like. In some embodiments, the kit is suitable for any of the methods described herein. In some embodiments, the kit comprises one or more elements, compositions (e.g., pharmaceutical compositions), Gene Writers, and/or Gene Writer systems, or a functional fragment or component thereof, e.g., disposed in an article of manufacture. In some embodiments, the kit comprises instructions for use thereof.

In an aspect, the disclosure provides an article of manufacture, e.g., in which a kit as described herein, or a component thereof, is disposed.

In an aspect, the disclosure provides a pharmaceutical composition comprising a Gene Writer or a Gene Writing system, e.g., as described herein. In some embodiments, the pharmaceutical composition further comprises a pharmaceutically acceptable carrier or excipient.

In some embodiments, the pharmaceutical composition comprises a template RNA and/or an RNA encoding the polypeptide. In embodiments, the pharmaceutical composition has one or more (e.g., 1, 2, 3, or 4) of the following characteristics:
(a) less than 1% (e.g., less than 0.5%, 0.4%, 0.3%, 0.2%, or 0.1%) DNA template relative to the template RNA and/or the RNA encoding the polypeptide, e.g., on a molar basis;
(b) less than 1% (e.g., less than 0.5%, 0.4%, 0.3%, 0.2%, or 0.1%) uncapped RNA relative to the template RNA and/or the RNA encoding the polypeptide, e.g., on a molar basis;
(c) less than 1% (e.g., less than 0.5%, 0.4%, 0.3%, 0.2%, or 0.1%) partial length RNAs relative to the template RNA and/or the RNA encoding the polypeptide, e.g., on a molar basis;
(d) substantially lacks unreacted cap dinucleotides.

### Chemistry, Manufacturing, and Controls (CMC):

Purification of protein therapeutics is described, for example, in Franks, Protein Biotechnology: Isolation, Characterization, and Stabilization, Humana Press (2013); and in Cutler, Protein Purification Protocols (Methods in Molecular Biology), Humana Press (2010).

In some embodiments, a Gene Writer™ system, polypeptide or nucleic acid encoding a polypeptide (e.g., mRNA), and/or template nucleic acid (e.g., template RNA) conforms to certain quality standards. In some embodiments, a Gene Writer™ system, polypeptide or nucleic acid encoding a polypeptide (e.g., mRNA), and/or template nucleic acid (e.g., template RNA) produced by a method described herein conforms to certain quality standards. Accordingly, the disclosure is directed, in some aspects, to methods of manufacturing a Gene Writer™ system, polypeptide or nucleic acid encoding a polypeptide (e.g., mRNA), and/or template nucleic acid (e.g., template RNA) that conforms to certain quality standards, e.g., in which said quality standards are assayed. The disclosure is also directed, in some aspects, to methods of assaying said quality standards in a Gene Writer™ system, polypeptide or nucleic acid encoding a polypeptide (e.g., mRNA), and/or template nucleic acid (e.g., template RNA). In some embodiments, quality standards include, but are not limited to:
(i) the length of an RNA, e.g., an mRNA encoding the GeneWriter polypeptide or a Template RNA, e.g., whether the RNA has a length that is above a reference length or within a reference length range, e.g., whether at least 80, 85, 90, 95, 96, 97, 98, or 99% of the RNA present is greater than 1000, 2000, 3000, 4000, or 5000 nucleotides long;
(ii) the presence, absence, and/or length of LTRs, e.g., 5′ or 3′ LTRs, in a Template RNA, e.g., whether at least 80, 85, 90, 95, 96, 97, 98, or 99% of the Template RNA present contains full-length 5′ and 3′ LTRs;
(iii) the presence, absence, and/or length of a polyA tail on the RNA, e.g., whether at least 80, 85, 90, 95, 96, 97, 98, or 99% of the mRNA, or Template RNA, where applicable, present contains a polyA tail (e.g., a polyA tail that is at least 5, 10, 20, 30, 50, 70, 100 nucleotides in length);
(iv) the presence, absence, and/or type of a 5′ cap on the RNA, e.g., whether at least 80, 85, 90, 95, 96, 97, 98, or 99% of the mRNA, or Template RNA, where applicable, present contains a 5′ cap, e.g., whether that cap is a 7-methylguanosine cap, e.g., a 0-Me-m7G cap;
(v) the presence, absence, and/or type of one or more modified nucleotides (e.g., selected from pseudouridine, dihydrouridine, inosine, 7-methylguanosine, 1-N-methylpseudouridine (1-Me-Ψ), 5-methoxyuridine (5-MO-U), 5-methylcytidine (5mC), or a locked nucleotide) in the RNA, e.g., whether at least 80, 85, 90, 95, 96, 97, 98, or 99% of the RNA present contains one or more modified nucleotides;
(vi) the stability of the RNA (e.g., over time and/or under a pre-selected condition), e.g., whether at least 80, 85, 90, 95, 96, 97, 98, or 99% of the RNA remains intact (e.g., greater than 1000, 2000, 3000, 4000, or 5000 nucleotides long) after a stability test; or
(vii) the potency of the RNA in a system for modifying DNA, e.g., whether at least 1%, 2%, 3%, 4%, 5%, 6%, 7%, 8%, 9%, 10%, 15%, 20%, or at least 25% of cells are modified after a system comprising the RNA is assayed for potency.
(viii) the length of the polypeptide, first polypeptide, or second polypeptide, e.g., whether the polypeptide, first polypeptide, or second polypeptide has a length that is above a reference length or within a reference length range, e.g., whether at least 80, 85, 90, 95, 96, 97, 98, or 99% of the polypeptide, first polypeptide, or second polypeptide present is greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long);
(ix) the presence, absence, and/or type of post-translational modification on the polypeptide, first polypeptide, or second polypeptide, e.g., whether at least 80, 85, 90, 95, 96, 97, 98, or 99% of the polypeptide, first polypeptide, or second polypeptide contains phosphorylation, methylation, acetylation, myristoylation, palmitoylation, isoprenylation, glipyatyon, or lipoylation, or any combination thereof,
(x) the presence, absence, and/or type of one or more artificial, synthetic, or non-canonical amino acids (e.g., selected from ornithine, β-alanine, GABA, δ-Aminolevulinic acid, PABA, a D-amino acid (e.g., D-alanine or D-glutamate), aminoisobutyric acid, dehydroalanine, cystathionine, lanthionine, Djenkolic acid, Diaminopimelic acid, Homoalanine, Norvaline, Norleucine, Homonorleucine, homoserine, O-methyl-homoserine and O-ethyl-homoserine, ethionine, selenocysteine, selenohomocysteine, selenomethionine, selenoethionine, tellurocysteine, or telluromethionine) in the polypeptide, first polypeptide, or second polypeptide, e.g., whether at least 80, 85, 90, 95, 96, 97, 98, or 99% of the polypeptide, first polypeptide, or second polypeptide present contains one or more artificial, synthetic, or non-canonical amino acids;
(xi) the stability of the polypeptide, first polypeptide, or second polypeptide (e.g., over time and/or under a pre-selected condition), e.g., whether at least 80, 85, 90, 95, 96, 97, 98, or 99% of the polypeptide, first polypeptide, or second polypeptide remains intact (e.g., greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long)) after a stability test;
(xii) the potency of the polypeptide, first polypeptide, or second polypeptide in a system for modifying DNA, e.g., whether at least 1% of target sites are modified after a system comprising the polypeptide, first polypeptide, or second polypeptide is assayed for potency; or
(xiii) the presence, absence, and/or level of one or more of a pyrogen, virus, fungus, bacterial pathogen, or host cell protein, e.g., whether the system is free or substantially free of pyrogen, virus, fungus, bacterial pathogen, or host cell protein contamination.

In some embodiments, a system or pharmaceutical composition described herein is endotoxin free.

In some embodiments, the presence, absence, and/or level of one or more of a pyrogen, virus, fungus, bacterial pathogen, and/or host cell protein is determined. In embodiments, whether the system is free or substantially free of pyrogen, virus, fungus, bacterial pathogen, and/or host cell protein contamination is determined.

In some embodiments, a pharmaceutical composition or system as described herein has one or more (e.g., 1, 2, 3, or 4) of the following characteristics:
(a) less than 1% (e.g., less than 0.5%, 0.4%, 0.3%, 0.2%, or 0.1%) DNA template relative to the template RNA and/or the RNA encoding the polypeptide, e.g., on a molar basis;
(b) less than 1% (e.g., less than 0.5%, 0.4%, 0.3%, 0.2%, or 0.1%) uncapped RNA relative to the template RNA and/or the RNA encoding the polypeptide, e.g., on a molar basis;
(c) less than 1% (e.g., less than 0.5%, 0.4%, 0.3%, 0.2%, or 0.1%) partial length RNAs relative to the template RNA and/or the RNA encoding the polypeptide, e.g., on a molar basis;
(d) substantially lacks unreacted cap dinucleotides.

### Regulation of System Components

It is highly desirable for a Gene Writer system of this invention to exhibit activity in target cells, while simultaneously having reduced activity in non-target cells. Thus, regulatory control of one or more components of the system is contemplated in preferred embodiments.

### Promoters and Enhancers:

In some embodiments, a nucleic acid described herein (e.g., a nucleic acid encoding a Gene Writer polypeptide, Template RNA, or an open reading frame in a heterologous object sequence) comprises a promoter sequence, e.g., a tissue specific promoter sequence. In some embodiments, the tissue-specific promoter is used to increase the target-cell specificity of a GeneWriter system. For instance, the promoter can be chosen on the basis that it is active in a target cell type but not active in (or active at a lower level in) a non-target cell type. Thus, a tissue-specific promoter used to drive expression of a nucleic acid encoding a Gene Writer polypeptide or Template RNA would result in reduced expression of the component in non-target cells, leading to a reduction in integration in non-target cells, as compared to target cells. In some embodiments, a tissue-specific promoter is used to drive expression of an open reading frame of a heterologous object sequence, such that even if heterologous object sequence integrated into the genome of a non-target cell, the promoter would not drive expression (or only drive low level expression) of the open reading frame.

In some embodiments, one or more promoter or enhancer elements are operably linked to a nucleic acid encoding a Gene Writer protein or a template nucleic acid, e.g., that controls expression of the heterologous object sequence. In certain embodiments, the one or more promoter or enhancer elements comprise cell-type or tissue specific elements. In some embodiments, the promoter or enhancer is the same or derived from the promoter or enhancer that naturally controls expression of the heterologous object sequence. For example, the ornithine transcarbomylase promoter and enhancer may be used to control expression of the ornithine transcarbomylase gene in a system or method provided by the invention for correcting ornithine transcarbomylase deficiencies. In some embodiments, the promoter is a promoter of Table 33 or a functional fragment or variant thereof.

Exemplary tissue specific promoters that are commercially available can be found, for example, at a uniform resource locator (e.g., https://www.invivogen.com/tissue-specific-promoters). In some embodiments, a promoter is a native promoter or a minimal promoter, e.g., which consists of a single fragment from the 5′ region of a given gene. In some embodiments, a native promoter comprises a core promoter and its natural 5′ UTR, In some embodiments, the 5′ UTR comprises an intron. In other embodiments, these include composite promoters, which combine promoter elements of different origins or were generated by assembling a distal enhancer with a minimal promoter of the same origin.

Exemplary cell or tissue specific promoters are provided in the tables, below, and exemplary nucleic acid sequences encoding them are known in the art and can be readily accessed using a variety of resources, such as the NCBI database, including RefSeq, as well as the Eukaryotic Promoter Database (//epd.epfl.ch//index.php).

TABLE 33
Exemplary cell or tissue-specific promoters

| Promoter | Target cells |
|---|---|
| B29 Promoter | B cells |
| CD14 Promoter | Monocytic Cells |
| CD43 Promoter | Leukocytes and platelets |
| CD45 Promoter | Hematopoeitic cells |
| CD68 promoter | macrophages |
| Desmin promoter | muscle cells |
| Elastase-1 promoter | pancreatic acinar cells |
| Endoglin promoter | endothelial cells |
| fibronectin promoter | differentiating cells, healing tissue |
| Flt-1 promoter | endothelial cells |
| GFAP promoter | Astrocytes |
| GPIIB promoter | megakaryocytes |
| ICAM-2 Promoter | Endothelial cells |
| INF-Beta promoter | Hematopoeitic cells |
| Mb promoter | muscle cells |
| Nphs1 promoter | podocytes |
| OG-2 promoter | Osteoblasts, Odonblasts |
| SP-B promoter | Lung |
| Syn1 promoter | Neurons |
| WASP promoter | Hematopoeitic cells |
| SV40/bAlb promoter | Liver |
| SV40/bAlb promoter | Liver |
| SV40/Cd3 promoter | Leukocytes and platelets |
| SV40/CD45 promoter | hematopoeitic cells |
| NSE/RU5′ promoter | Mature Neurons |

TABLE 34
Additional exemplary cell or tissue-specific promoters

| Promoter | Gene Description | Gene Specificity |
|---|---|---|
| APOA2 | Apolipoprotein A-II | Hepatocytes (from hepatocyte progenitors) |
| SERPINA 1 (hAAT) | Serpin peptidase inhibitor, clade A (alpha-1 antiproteinase, | Hepatocytes (from definitive endoderm stage) |
| | antitrypsin), member 1 (also named alpha 1 anti-tryps in) | |
| CYP3A | Cytochrome P450, family 3, subfamily A, polypeptide | Mature Hepatocytes |
| MIR122 | MicroRNA 122 | Hepatocytes (from early stage embryonic liver cells) |
| | | and endoderm |

Pancreatic specific promoters

| | | |
|---|---|---|
| INS | Insulin | Pancreatic beta cells (from definitive endoderm stage) |
| IRS2 | Insulin receptor substrate 2 | Pancreatic beta cells |
| Pdx1 | Pancreatic and duodenal homeobox 1 | Pancreas (from definitive endoderm stage) |
| Alx3 | Aristaless-like homeobox 3 | Pancreatic beta cells (from definitive endoderm stage) |
| Ppy | Pancreatic polypeptide | PP pancreatic cells (gamma cells) |

Cardiac specific promoters

| | | |
|---|---|---|
| Myh6 (aMHC) | Myosin, heavy chain 6, cardiac muscle, alpha | Late differentiation marker of cardiac muscle cells |
| | | (atrial specificity) |
| MYL2 (MLC-2v) | Myosin, light chain 2, regulatory, cardiac, slow | Late differentiation marker of cardiac muscle cells |
| | | (ventricular specificity) |
| NPPA (ANF) | Natriuretic peptide precursor A (also named Atrial | Atrial specificity in adult cells |
| | Natriuretic Factor) | |
| Slc8a1 (Ncx1) | Solute carrier family 8 (sodium/calcium exchanger), | Cardiomyocytes from early developmental stages |
| | member 1 | |

CNS specific promoters

| | | |
|---|---|---|
| SYN1 (hSyn) | Synapsin I | Neurons |
| GFAP | Glial fibrillary acidic protein | Astrocytes |
| INA | Internexin neuronal intermediate filament protein, | Neuroprogenitors |
| | alpha (a-internexin) | |
| NES | Nestin | Neuroprogenitors and ectoderm |
| MOBP | Myelin-associated oligodendrocyte basic protein | Oligodendrocytes |
| MBP | Myelin basic protein | Oligodendrocytes |
| TH | Tyrosine hydroxylase | Dopaminergic neurons |
| FOXA2 (HNF3 beta) | Forkhead box A2 | Dopaminergic neurons (also used as a marker of endoderm) |

Skin specific promoters

| | | |
|---|---|---|
| FLG | Filaggrin | Keratinocytes from granular layer |
| TGM3 | Transglutaminase 3 | Keratinocytes from granular layer |

Immune cell specific promoters
Urogential cell specific promoters

| | | |
|---|---|---|
| Pbsn | Probasin | Prostatic epithelium |
| Upk2 | Uroplakin 2 | Bladder |
| Sbp | Spermine binding protein | Prostate |
| Fer114 | Fer-1-like 4 | Bladder |

Endothelial cell specific promoters

| | | |
|---|---|---|
| ENG | Endoglin | Endothelial cells |

Pluripotent and embryonic cell specific promoters

| | | |
|---|---|---|
| Synthetic Oct4 | Synthetic promoter based on a Oct-4 core enhancer element | Pluripotent cells (ES cells, iPS cells) |
| T brachyury | Brachyury | Mesoderm |
| NES | Nestin | Neuroprogenitors and Ectoderm |
| SOX17 | SRY (sex determining region Y)-box 17 | Endoderm |
| FOXA2 (HNFJ beta) | Forkhead box A2 | Endoderm (also used as a marker of dopaminergic neurons) |

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544: incorporated herein by reference in its entirety).

In some embodiments, a nucleic acid encoding a Gene Writer or template nucleic acid is operably linked to a control element, e.g., a transcriptional control element, such as a promoter. The transcriptional control element may, in some embodiment, be functional in either a eukaryotic cell, e.g., a mammalian cell; or a prokaryotic cell (e.g., bacterial or archaeal cell). In some embodiments, a nucleotide sequence encoding a polypeptide is operably linked to multiple control elements, e.g., that allow expression of the nucleotide sequence encoding the polypeptide in both prokaryotic and eukaryotic cells.

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter, a neurofilament promoter (see, e.g., GenBank HUMNFL, L04147), a synapsin promoter (see, e g., GenBank HUMSYNIB, M55301); a thy-1 promoter (see, e.g., Chen et al. (1987) Cell 51:7-19: and Llewellyn, et al. (2010) Nat. Med. 16(10):1161-1166); a serotonin receptor promoter (see, e g., GenBank S62283); a tyrosine hydroxylase promoter (TH) (see, e.g., Oh et al. (2009) Gene Ther 16:437; Sasaoka et al. (1992) Mol. Brain Res. 16:274: Boundy et al (1998) J. Neurosci. 18:9989; and Kaneda et al. (1991) Neuron 6:583-594); a GnRH promoter (see, e.g., Radovick et al. (1991) Proc. Natd. Acad. Sci. USA 88:3402-3406); an L7 promoter (see, e.g., Oberdick et al. (1990) Science 248.223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805): a myelin basic protein (MBP) promoter; a Ca2+-calmodulin-dependent protein kinase II-alpha (CamKIIα) promoter (see, e.g., Mayford et al. (1996) Proc. Natl. Acad. Sci. USA 93:13250; and Casanova et al (2001) Genesis 31:37); a CMV enhancer/platelet-derived growth factor-β promoter (see, e.g., Liu et al. (2004) Gene Therapy 11:52-60); and the like.

Adipocyte-specific spatially restricted promoters include, but are not limited to, the aP2 gene promoter/enhancer. e.g., a region from −5.4 kb to +21 bp of a human aP2 gene (see, e.g., Tozzo et al. (1997) Endocrinol. 381604; Ross et al. (1990) Proc. Natd. Acad. Sci USA 87:9590; and Pavjani et al. (2005) Nat. Med. 11:797); a glucose transporter-4 (GLUT4) promoter (see, e g., Knight et al. (2003) Proc. Natl. Acad. Sci. USA 100.14725); a fatty acid translocase (FAT/CD36) promoter (see, e.g., Kuriki et al. (2002) Biol. Pharm. Bull. 25:1476; and Sato et al. (2002) J Biol Chem 277.15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013, and Chen et al. (1999) Biochem. Biophys. Res. Comm. 262:187); an adiponectin promoter (see, e.g., Kita et al. (2005) Biochem. Biophys. Res Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e g., Seo et al. (2003) Molec Endocrinol 17.1522); and the like.

Cardiomyocyte-specific spatially restricted promoters include, but are not limited to, control sequences derived from the following genes: myosin light chain-2, α-myosin heavy chain, AE3, cardiac troponin C, cardiac actin, and the like. Franz et al (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22.608-617; and Sartorelli et al. (1992) Proc Natl Acad. Sci. USA 89:4047-4051.

Smooth muscle-specific spatially restricted promoters include, but are not limited to, an SM22α promoter (see, e g., Akyurek et al. (2000) Mol. Med. 6:983; and U.S. Pat. No. 7,169,874); a smoothelin promoter (see, e.g., WO 2001/018048); an α-smooth muscle actin promoter; and the like. For example, a 0.4 kb region of the SM22α promoter, within which lie two CArG elements, has been shown to mediate vascular smooth muscle cell-specific expression (see, e.g., Kim, et al. (1997) Mol. Cell Biol. 17, 2266-2278, Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter, a rhodopsin kinase promoter (Young et al (2003) Ophthalmol Vis. Sci 44:4076); a beta phosphodiesterase gene promoter (Nicoud et al. (2007) J. Gene Med. 9:1015); a retinitis pigmentosa gene promoter (Nicoud et al (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

Cell-specific promoters known in the art may be used to direct expression of a Gene Writer protein, e.g., as described herein. Nonlimiting exemplary mammalian cell-specific promoters have been characterized and used in mice expressing Cre recombinase in a cell-specific manner. Certain nonlimiting exemplary mammalian cell-specific promoters are listed in Table 1 of U.S. Pat. No. 9,845,481, incorporated herein by reference.

In some embodiments, a cell-specific promoters is a promoter that is active in plants. Many exemplary cell-specific plant promoters are known in the art See, e g., U.S. Pat. Nos. 5,097,025; 5,783,393; 5,880,330; 5,981,727; 7,557,264; 6,291,666; 7,132,526; and 7,323,622; and U.S. Publication Nos. 2010/0269226; 2007/0180580, 2005/0034192: and 2005/0086712, which are incorporated by reference herein in their entireties for any purpose.

In some embodiments, a vector as described herein comprises an expression cassette. The term “expression cassette”, as used herein, refers to a nucleic acid construct comprising nucleic acid elements sufficient for the expression of the nucleic acid molecule of the instant invention. Typically, an expression cassette comprises the nucleic acid molecule of the instant invention operatively linked to a promoter sequence. The term“operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e g., the coding sequence is under the transcriptional control of the promoter). Encoding sequences can be operatively linked to regulatory sequences in sense or antisense orientation. In certain embodiments, the promoter is a heterologous promoter. The term“heterologous promoter”, as used herein, refers to a promoter that is not found to be operatively linked to a given encoding sequence in nature. In certain embodiments, an expression cassette may comprise additional elements, for example, an intron, an enhancer, a polyadenylation site, a woodchuck response element (WRE), and/or other elements known to affect expression levels of the encoding sequenceA“promoter” typically controls the expression of a coding sequence or functional RNA. In certain embodiments, a promoter sequence comprises proximal and more distal upstream elements and can further comprise an enhancer element. An “enhancer” can typically stimulate promoter activity and may be an innate element of the promoter or a heterologous element inserted to enhance the level or tissue-specificity of a promoter. In certain embodiments, the promoter is derived in its entirety from a native gene. In certain embodiments, the promoter is composed of different elements derived from different naturally occurring promoters. In certain embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known to those of skill in the art. Examples of promoter include, but are not limited to, the phosphoglycerate kinase (PKG) promoter, CAG (composite of the CMV enhancer the chicken beta actin promoter (CBA) and the rabbit beta globin intron.), NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerate kinase promoter, the human alpha-1 antitrypsin (hAAT) promoter, the transthyretin promoter, the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter, all of which are promoters well known and readily available to those of skill in the art, can be used to obtain high-level expression of the coding sequence of interest. In addition, sequences derived from non-viral genes, such as the murine metallothionein gene, will also find use herein. Such promoter sequences are commercially available from, e.g., Stratagene (San Diego, CA). Additional exemplary promoter sequences are described, for example, in WO2018213786A1 (incorporated by reference herein in its entirety).

In some embodiments, the apolipoprotein E enhancer (ApoE) or a functional fragment thereof is used, e.g., to drive expression in the liver. In some embodiments, two copies of the ApoE enhancer or a functional fragment thereof is used. In some embodiments, the ApoE enhancer or functional fragment thereof is used in combination with a promoter, e.g., the human alpha-1 antitrypsin (hAAT) promoter.

In some embodiments, the regulatory sequences impart tissue-specific gene expression capabilities in some cases, the tissue-specific regulatory sequences bind tissue-specific transcription factors that induce transcription in a tissue specific manner. Various tissue-specific regulatory sequences (e g., promoters, enhancers, etc.) are known in the art. Exemplary tissue-specific regulatory sequences include, but are not limited to, the following tissue-specific promoters: a liver-specific thyroxin binding globulin (TBG) promoter, a insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996): alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 5.373-84 (1995)), and others. Additional exemplary promoter sequences are described, for example, in U.S. patent Ser. No. 10/300,146 (incorporated herein by reference in its entirety). In some embodiments, a tissue-specific regulatory element, e.g., a tissue-specific promoter, is selected from one known to be operably linked to a gene that is highly expressed in a given tissue, e.g., as measured by RNA-seq or protein expression data, or a combination thereof. Methods for analyzing tissue specificity by expression are taught in Fagerberg et al. Mol Cell Proteomics 13(2):397-406 (2014), which is incorporated herein by reference in its entirety.

In some embodiments, a vector described herein is a multicistronic expression construct. Multicistronic expression constructs include, for example, constructs harboring a first expression cassette, e.g. comprising a first promoter and a first encoding nucleic acid sequence, and a second expression cassette, e.g. comprising a second promoter and a second encoding nucleic acid sequence. Such multicistronic expression constructs may, in some instances, be particularly useful in the delivery of non-translated gene products, such as hairpin RNAs, together with a polypeptide, for example, a Gene Writer polypeptide and Gene Writer template. In some embodiments, multicistronic expression constructs may exhibit reduced expression levels of one or more of the included transgenes, for example, because of promoter interference or the presence of incompatible nucleic acid elements in close proximity. If a multicistronic expression construct is part of a viral vector, the presence of a self-complementary nucleic acid sequence may, in some instances, interfere with the formation of structures necessary for viral reproduction or packaging.

In some embodiments, the sequence encodes an RNA with a hairpin. In some embodiments, the hairpin RNA is a guide RNA, a template RNA, shRNA, or a microRNA. In some embodiments, the first promoter is an RNA polymerase I promoter. In some embodiments, the first promoter is an RNA polymerase 11 promoter. In some embodiments, the second promoter is an RNA polymerase III promoter. In some embodiments, the second promoter is a U6 or H1 promoter. In some embodiments, the nucleic acid construct comprises the structure of AAV construct B1 or B2.

Without wishing to be bound by theory, multicistronic expression constructs may not achieve optimal expression levels as compared to expression systems containing only one cistron One of the suggested causes of lower expression levels achieved with multicistronic expression constructs comprising two or more promoter elements is the phenomenon of promoter interference (see, e.g., Curtin J A, Dane A P, Swanson A, Alexander i E, Ginn S L. Bidirectional promoter interference between two widely used internal heterologous promoters in a late-generation lentiviral construct. Gene Ther. 2008 March; 15(5):384-90: and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15(10):995-1002; both references incorporated herein by reference for disclosure of promoter interference phenomenon). In some embodiments, the problem of promoter interference may be overcome, e.g., by producing multicistronic expression constructs comprising only one promoter driving transcription of multiple encoding nucleic acid sequences separated by internal ribosomal entry sites, or by separating cistrons comprising their own promoter with transcriptional insulator elements. In some embodiments, single-promoter driven expression of multiple cistrons may result in uneven expression levels of the cistrons. In some embodiments, a promoter cannot efficiently be isolated and isolation elements may not be compatible with some gene transfer vectors, for example, some retroviral vectors.

miRNAs, Inhibitors, and miRNA Binding Sites:

miRNAs and other small interfering nucleic acids generally regulate gene expression via target RNA transcript cleavage/degradation or translational repression of the target messenger RNA (mRNA). miRNAs may, in some instances, be natively expressed, typically as final 19-25 non-translated RNA products. miRNAs generally exhibit their activity through sequence-specific interactions with the 3′ untranslated regions (UTR) of target mRNAs. These endogenously expressed miRNAs may form hairpin precursors that are subsequently processed into an miRNA duplex, and further into a mature single stranded miRNA molecule. This mature miRNA generally guides a multiprotein complex, miRISC, which identifies target 3′ UTR regions of target mRNAs based upon their complementarity to the mature miRNA. Useful transgene products may include, for example, miRNAs or miRNA binding sites that regulate the expression of a linked polypeptide. A non-limiting list of miRNA genes; the products of these genes and their homologues are useful as transgenes or as targets for small interfering nucleic acids (e.g., miRNA sponges, antisense oligonucleotides), e.g., in methods such as those listed in U.S. Ser. No. 10/300,146, 22.25-25:48, incorporated by reference. In some embodiments, one or more binding sites for one or more of the foregoing miRNAs are incorporated in a transgene, e.g., a transgene delivered by a rAAV vector, e.g., to inhibit the expression of the transgene in one or more tissues of an animal harboring the transgene. In some embodiments, a binding site may be selected to control the expression of a trangene in a tissue specific manner. For example, binding sites for the liver-specific miR-122 may be incorporated into a transgene to inhibit expression of that transgene in the liver. Additional exemplary miRNA sequences are described, for example, in U.S. patent Ser. No. 10/300,146 (incorporated herein by reference in its entirety). For liver-specific Gene Writing, however, overexpression of miR-122 may be utilized instead of using binding sites to effect miR-122-specific degradation. This miRNA is positively associated with hepatic differentiation and maturation, as well as enhanced expression of liver specific genes. Thus, in some embodiments, the coding sequence for miR-122 may be added to a component of a Gene Writing system to enhance a liver-directed therapy.

A miR inhibitor or miRNA inhibitor is generally an agent that blocks miRNA expression and/or processing. Examples of such agents include, but are not limited to, microRNA antagonists, microRNA specific antisense, microRNA sponges, and microRNA oligonucleotides (double-stranded, hairpin, short oligonucleotides) that inhibit miRNA interaction with a Drosha complex. MicroRNA inhibitors, e.g., miRNA sponges, can be expressed in cells from transgenes (e.g., as described in Ebert, M S. Nature Methods, Epub Aug. 12, 2007; incorporated by reference herein in its entirety). In some embodiments, microRNA sponges, or other miR inhibitors, are used with the AAVs. microRNA sponges generally specifically inhibit miRNAs through a complementary heptameric seed sequence. In some embodiments, an entire family of miRNAs can be silenced using a single sponge sequence. Other methods for silencing miRNA function (derepression of miRNA targets) in cells will be apparent to one of ordinary skill in the art.

In some embodiments, a miRNA as described herein comprises a sequence listed in Table 4 of PCT Publication No. WO2020014209, incorporated herein by reference. Also incorporated herein by reference are the listing of exemplary miRNA sequences from WO2020014209.

In some embodiments, it is advantageous to silence one or more components of a Gene Writing system (e.g., mRNA encoding a Gene Writer polypeptide, a Gene Writer Template RNA, or a heterologous object sequence expressed from the genome after successful Gene Writing) in a portion of cells. In some embodiments, it is advantageous to restrict expression of a component of a Gene Writing system to select cell types within a tissue of interest.

For example, it is known that in a given tissue, e.g., liver, macrophages and immune cells, e.g., Kupffer cells in the liver, may engage in uptake of a delivery vehicle for one or more components of a Gene Writing system. In some embodiments, at least one binding site for at least one miRNA highly expressed in macrophages and immune cells, e.g., Kupffer cells, is included in at least one component of a Gene Writing system, e.g., nucleic acid encoding a Gene Writing polypeptide or a transgene. In some embodiments, a miRNA that targets the one or more binding sites is listed in a table referenced herein, e.g., miR-142, e.g., mature miRNA hsa-miR-142-5p or hsa-miR-142-3p.

In some embodiments, there may be a benefit to decreasing Gene Writer levels and/or Gene Writer activity in cells in which Gene Writer expression or overexpression of a transgene may have a toxic effect. For example, it has been shown that delivery of a transgene overexpression cassette to dorsal root ganglion neurons may result in toxicity of a gene therapy (see Hordeaux et al Sci Transl Med 12(569):eaba9188 (2020), incorporated herein by reference in its entirety). In some embodiments, at least one miRNA binding site may be incorporated into a nucleic acid component of a Gene Writing system to reduce expression of a system component in a neuron, e.g., a dorsal root ganglion neuron. In some embodiments, the at least one miRNA binding site incorporated into a nucleic acid component of a Gene Writing system to reduce expression of a system component in a neuron is a binding site of miR-182, e.g., mature miRNA hsa-miR-182-5p or hsa-miR-182-3p. In some embodiments, the at least one miRNA binding site incorporated into a nucleic acid component of a Gene Writing system to reduce expression of a system component in a neuron is a binding site of miR-183, e.g., mature miRNA hsa-miR-183-5p or hsa-miR-183-3p. In some embodiments, combinations of miRNA binding sites may be used to enhance the restriction of expression of one or more components of a Gene Writing system to a tissue or cell type of interest.

Table A5 below provides exemplary miRNAs and corresponding expressing cells, e.g., a miRNA for which one can, in some embodiments, incorporate binding sites (complementary sequences) in the transgene or polypeptide nucleic acid, e.g., to decrease expression in that off-target cell.

TABLE A5
Exemplary miRNA from off-target cells and tissues

| | miRNA | | | |
|---|---|---|---|---|
| Silenced cell type | name | Mature miRNA | miRNA sequence | SEQ ID NO: |
| Kupffer cells | miR-142 | hsa-miR-142-5p | cauaaaguagaaagcacuacu | 134 |
| Kupffer cells | miR-142 | hsa-miR-142-3p | uguaguguuuccuacuuuaugga | 135 |
| Dorsal root | miR-182 | hsa-miR-182-5p | uuuggcaaugguagaacucacacu | 136 |
| ganglion neurons | | | | |
| Dorsal root | miR-182 | hsa-miR-182-3p | ugguucuagacuugccaacua | 137 |
| ganglion neurons | | | | |
| Dorsal root | miR-183 | hsa-miR-183-5p | uauggcacugguagaauucacu | 138 |
| ganglion neurons | | | | |
| Table XDorsal | miR-183 | hsa-miR-183-3p | gugaauuaccgaagggccauaa | 139 |
| root ganglion | | | | |
| neurons | | | | |
| Hepatocytes | miR-122 | hsa-miR-122-5p | uggagugugacaaugguguuug | 140 |
| Hepatocytes | miR-122 | hsa-miR-122-3p | aacgccauuaucacacuaaaua | 141 |

In some embodiments, a nucleic acid described herein (e.g., a nucleic acid encoding a Gene Writer polypeptide, Gene Writer Template, and/or an open reading frame in a heterologous object sequence) comprises at least one microRNA binding site. In some embodiments, the microRNA binding site is used to increase the target-cell specificity of a Gene Writer system. For instance, the microRNA binding site can be chosen on the basis that it is recognized by a miRNA that is present in a non-target cell type, but that is not present (or is present at a reduced level relative to the non-target cell) in a target cell type. Thus, when the RNA (e.g., the RNA encoding a Gene Writer polypeptide, Gene Writer Template, and/or transcript from an open reading frame in a heterologous object sequence) is present in a non-target cell, it would be bound by the miRNA, and when the RNA (e.g., the RNA encoding a Gene Writer polypeptide, Gene Writer Template, and/or transcript from an open reading frame in a heterologous object sequence) is present in a target cell, it would not be bound by the miRNA (or bound but at reduced levels relative to the non-target cell). While not wishing to be bound by theory, binding of the miRNA to an RNA of the system (e.g., the RNA encoding a Gene Writer polypeptide, Gene Writer Template, and/or transcript from an open reading frame in a heterologous object sequence) may result in destabilization or degradation of the RNA molecule or interference with translation of a coding RNA. Accordingly, the heterologous object sequence would be inserted into the genome of target cells more efficiently than into the genome of non-target cells. It is contemplated that incorporation of one or more appropriate miRNA binding sites into a nucleic acid encoding the Gene Writer polypeptide or Template RNA would thus reduce integration in off-target cells, while incorporation into a heterologous object sequence would reduce expression of a transgene in off-target cells. A system having a microRNA binding site in a nucleic acid would be expected to exhibit increased specificity for target cells by the addition of more miRNA binding sites on the same or on an additional nucleic acid component of the system. In some embodiments, one or more component of a Gene Writing system comprises one or more miRNA binding sites to reduce activity in off-target cells.

In some embodiments, a system comprising one or more tissue-specific promoter sequences may be used in combination with one or more microRNA binding sites, e.g., as described herein. When used in combination, it is contemplated that the one or more tissue-specific promoters would drive lower transcription of operably linked open reading frames, while one or more miRNA binding sites would simultaneously reduce the stability and/or translation of the comprising transcripts, leading to highly reduced activity of a Gene Writer system in one or more non-target cells.

In some embodiments, a heterologous object sequence comprised by a template RNA (or DNA encoding the template RNA) is operably linked to at least one regulatory sequence. In some embodiments, the heterologous object sequence is operably linked to a tissue-specific promoter, such that expression of the heterologous object sequence, e.g., a therapeutic protein, is upregulated in target cells, as above. In some embodiments, the heterologous object sequence is operably linked to a miRNA binding site, such that expression of the heterologous object sequence, e.g., a therapeutic protein, is downregulated in cells with higher levels of the corresponding miRNA, e.g., non-target cells, as above.

### Small Molecule Regulation

In some embodiments a polypeptide described herein (e.g., a Gene Writer polypeptide, or a domain or variant thereof) is controllable via a small molecule. In some embodiments the polypeptide is dimerized via a small molecule.

In some embodiment, the polypeptide is controllable via Chemical Induction of Dimerization (CID) with small molecules. CID is generally used to generate switches of protein function to alter cell physiology. An exemplary high specificity, efficient dimerizer is rimiducid (AP1903), which has two identical, protein-binding surfaces arranged tail-to-tail, each with high affinity and specificity for a mutant of FKBP12: FKBP12(F36V) (FKBP12v36, Fv36 or Fv), Attachment of one or more Fv domains onto one or more cell signaling molecules that normally rely on homodimerization can convert that protein to rimiducid control. Homodimerization with rimiducid is used in the context of an inducible caspase safety switch. This molecular switch that is controlled by a distinct dimerizer ligand, based on the heterodimerizing small molecule, rapamycin, or rapamycin analogs (“rapalogs”). Rapamycin binds to FKBP12, and its variants, and can induce heterodimerization of signaling domains that are fused to FKBP12 by binding to both FKBP12 and to polypeptides that contain the FKBP-rapamycin-binding (FRB) domain of mTOR. Provided in some embodiments of the present application are molecular switches that greatly augment the use of rapamycin, rapalogs and rimiducid as agents for therapeutic applications.

In some embodiments of the dual switch technology, a homodimerizer, such as AP1903 (rimiducid), directly induces dimerization or multimerization of polypeptides comprising an FKBP12 multimerizing region. In other embodiments, a polypeptide comprising an FKBP12 multimerization is multimerized, or aggregated by binding to a heterodimerizer, such as rapamycin or a rapalog, which also binds to an FRB or FRB variant multimerizing region on a chimeric polypeptide, also expressed in the modified cell, such as, for example, a chimeric antigen receptor. Rapamycin is a natural product macrolide that binds with high affinity (<1 nM) to FKBP12 and together initiates the high-affinity, inhibitory interaction with the FKBP-Rapamycin-Binding (FRB) domain of mTOR. FRB is small (89 amino acids) and can thereby be used as a protein “tag” or “handle” when appended to many proteins. Coexpression of a FRB-fused protein with a FKBP12-fused protein renders their approximation rapamycin-inducible (12-16). This can serve as the basis for a cell safety switch regulated by the orally available ligand, rapamycin, or derivatives of rapamycin (rapalogs) that do not inhibit mTOR at a low, therapeutic dose but instead bind with selected, Caspase-9-fused mutant FRB domains. (see Sabatini D N, et al., Cell. 1994; 78(1):35-43; Brown E J, et al., Nature. 1994; 369(6483):756-8; Chen J, et al., Proc Natl Acad Sci USA. 1995; 92(11):4947-51; and Choi J, Science. 1996; 273(5272):239-42).

In some embodiments, two levels of control are provided in the therapeutic cells. In embodiments, the first level of control may be tunable, i.e., the level of removal of the therapeutic cells may be controlled so that it results in partial removal of the therapeutic cells. In some embodiments, the chimeric antigen polypeptide comprises a binding site for rapamycin, or a rapamycin analog. In embodiments, also present in the therapeutic cell is a suicide gene, such as, for example, one encoding a caspase polypeptide. Using this controllable first level, the need for continued therapy may, in some embodiments, be balanced with the need to eliminate or reduce the level of negative side effects. In some embodiments, a rapamycin analog, a rapalog is administered to the patient, which then binds to both the caspase polypeptide and the chimeric antigen receptor, thus recruiting the caspase polypeptide to the location of the CAR, and aggregating the caspase polypeptide. Upon aggregation, the caspase polypeptide induces apoptosis. The amount of rapamycin or rapamycin analog administered to the patient may vary, if the removal of a lower level of cells by apoptosis is desired in order to reduce side effects and continue CAR therapy, a lower level of rapamycin or rapamycin may be administered to the patient. In some embodiments, the second level of control may be designed to achieve the maximum level of cell elimination. This second level may be based, for example, on the use of rimiducid, or AP1903. If there is a need to rapidly eliminate up to 100% of the therapeutic cells, the AP1903 may be administered to the patient. The multimeric AP1903 binds to the caspase polypeptide, leading to multimerization of the caspase polypeptide and apoptosis. In certain examples, second level may also be tunable, or controlled, by the level of AP1903 administered to the subject.

In certain embodiments, small molecules can be used to control genes, as described in for example, U.S. Ser. No. 10/584,351 at 47:53-56:47 (incorporated by reference herein in its entirety), together suitable ligands for the control features, e.g., in U.S. Ser. No. 10/584,351 at 56:48, et seq. as well as U10046049 at 43:27-52:20, incorporated by reference as well as the description of ligands for such control systems at 52:21, et seq.

### Modifications to Proteins of the System

### Subcellular Localization Signals:

In some embodiments, a polypeptide described herein (e.g., a Gene Writer polypeptide or a polypeptide encoded by a heterologous object sequence), comprises one or more (e.g., 2, 3, 4, 5) nuclear targeting sequences, for example, a nuclear localization sequence (NLS), e.g., as described above. In some embodiments, the NLS is a bipartite NLS. In some embodiments, an NLS facilitates the import of a protein comprising an NLS into the cell nucleus. In some embodiments, the NLS is fused to the N-terminus of a polypeptide described herein. In some embodiments, the NLS is fused to the C-terminus of a polypeptide described herein. In some embodiments, the NLS is fused to the N-terminus or the C-terminus of a polypeptide or domain described herein. In some embodiments, a linker sequence is disposed between the NLS and the neighboring domain of a polypeptide described herein, e.g., a Gene Writer polypeptide.

In some embodiments, an NLS comprises the amino acid sequence MDSLLMNRRKFLYQFKNVRWAKGRRETYLC (SEQ ID NO: 142), PKKRKVEGADKRTADGSEFESPKKKRKV (SEQ ID NO: 143), RKSGKIAAIWKRPRKPKKKRKV KRTADGSEFESPKKKRKV (SEQ ID NO: 144), KKTELQTTNAENKTKKL (SEQ ID NO: 145), or KRGINDRNFWRGENGRKTR (SEQ ID NO: 146), KRPAATKKAGQAKKKK (SEQ ID NO: 147), or a functional fragment or variant thereof. Exemplary NLS sequences are also described in PCT/EP2000/011690, the contents of which are incorporated herein by reference for their disclosure of exemplary nuclear localization sequences. In some embodiments, an NLS comprises an amino acid sequence as disclosed in Table 8. An NLS of this table may be utilized with one or more copies in a polypeptide in one or more locations in a polypeptide, e.g., 1, 2, 3 or more copies of an NLS in an N-terminal domain, between peptide domains, in a C-terminal domain, or in a combination of locations, in order to improve subcellular localization to the nucleus. Multiple unique sequences may be used within a single polypeptide. Sequences may be naturally monopartite or bipartite, e.g., having one or two stretches of basic amino acids, or may be used as chimeric bipartite sequences. Sequence references correspond to UniProt accession numbers, except where indicated as SeqNLS for sequences mined using a subcellular localization prediction algorithm (Lin et al BMC Bioinformat 13:157 (2012), incorporated herein by reference in its entirety).

TABLE 8
Exemplary nuclear localization signals for use in Gene Writing systems

| Sequence | Sequence References | SEQ ID NO: |
|---|---|---|
| AHFKISGEKRPSTDPGKKAK | Q76IQ7 | 148 |
| NPKKKKKKDP | | |
| AHRAKKMSKTHA | P21827 | 149 |
| ASPEYVNLPINGNG | SeqNLS | 150 |
| CTKRPRW | O88622, Q86W56, Q9QYM2, O02776 | 151 |
| DKAKRVSRNKSEKKRR | O15516, Q5RAK8, Q91YB2, Q91YB0, | 152 |
| | Q8QGQ6, O08785, Q9WVS9, Q6YGZ4 | |
| EELRLKEELLKGIYA | Q9QY16, Q9UHL0, Q2TBP1, Q9QY15 | 153 |
| EEQLRRRKNSRLNNTG | G5EFF5 | 154 |
| EVLKVIRTGKRKKKAWKR | SeqNLS | 155 |
| MVTKVC | | |
| HHHHHHHHHHHHQPH | Q63934, G3V7L5, Q12837 | 156 |
| HKKKHPDASVNFSEFSK | P10103, Q4R844, P12682, B0CM99, | 157 |
| | A9RA84, Q6YKA4, P09429, P63159, | |
| | Q08IE6, P63158, Q9YH06, B1MTB0 | |
| HKRTKK | Q2R2D5 | 158 |
| IINGRKLKLKKSRRRSSQTS | SeqNLS | 159 |
| NNSFTSRRS | | |
| KAEQERRK | Q8LH59 | 160 |
| KEKRKRREELFIEQKKRK | SeqNLS | 161 |
| KKGKDEWFSRGKKP | P30999 | 162 |
| KKGPSVQKRKKT | Q6ZN17 | 163 |
| KKKTVINDLLHYKKEK | SeqNLS, P32354 | 164 |
| KKNGGKGKNKPSAKIKK | SeqNLS | 165 |
| KKPKWDDFKKKKK | Q15397, Q8BKS9, Q562C7 | 166 |
| KKRKKD | SeqNLS, Q91Z62, Q1A730, Q969P5, | 167 |
| | Q2KHT6, Q9CPU7 | |
| KKRRKRRRK | SeqNLS | 168 |
| KKRRRRARK | Q9UMS6, D4A702, Q91YE8 | 169 |
| KKSKRGR | Q9UBS0 | 170 |
| KKSRKRGS | B4FG96 | 171 |
| KKSTALSRELGKIMRRR | SeqNLS, P32354 | 172 |
| KKSYQDPEIIAHSRPRK | Q9U7C9 | 173 |
| KKTGKNRKLKSKRVKTR | Q9Z301, O54943, Q8K3T2 | 174 |
| KKVSIAGQSGKLWRWKR | Q6YUL8 | 175 |
| KKYENVVIKRSPRKRGRPR | SeqNLS | 176 |
| K | | |
| KNKKRK | SeqNLS | 177 |
| KPKKKR | SeqNLS | 178 |
| KRAMKDDSHGNSTSPKRRK | Q0E671 | 179 |
| KRANSNLVAAYEKAKKK | P23508 | 180 |
| KRASEDTTSGSPPKKSSAGP | Q9BZZ5, Q5R644 | 181 |
| KR | | |
| KRFKRRWMVRKMKTKK | SeqNLS | 182 |
| KRGLNSSFETSPKKVK | Q8IV63 | 183 |
| KRGNSSIGPNDLSKRKQRK | SeqNLS | 184 |
| K | | |
| KRIHSVSLSQSQIDPSKKVK | SeqNLS | 185 |
| RAK | | |
| KRKGKLKNKGSKRKK | O15381 | 186 |
| KRRRRRRREKRKR | Q96GM8 | 187 |
| KRSNDRTYSPEEEKORRA | Q91ZF2 | 188 |
| KRTVATNGDASGAHRAKK | SeqNLS | 189 |
| MSK | | |
| KRVYNKGEDEQEHLPKGKK | SeqNLS | 190 |
| R | | |
| KSGKAPRRRAVSMDNSNK | Q9WVH4, O43524 | 191 |
| KVNFLDMSLDDIIIYKELE | Q9P127 | 192 |
| KVQHRIAKKTTRRRR | Q9DXE6 | 193 |
| LSPSLSPL | Q9Y261, P32182, P35583 | 194 |
| MDSLLMNRRKFLYQFKNVR | Q9GZX7 | 142 |
| WAKGRRETYLC | | |
| MPQNEYIELHRKRYGYRLD | SeqNLS | 195 |
| YHEKKRKKESREAHERSKK | | |
| AKKMIGLKAKLYHK | | |
| MVQLRPRASR | SeqNLS | 196 |
| NNKLLAKRRKGGASPKDDP | Q965G5 | 197 |
| MDDIK | | |
| NYKRPMDGTYGPPAKRHEG | O14497, A2BH40 | 198 |
| E | | |
| PDTKRAKLDSSETTMVKKK | SeqNLS | 199 |
| PEKRTKI | SegNLS | 200 |
| PGGRGKKK | Q719N1, Q9UBP0, A2VDN5 | 201 |
| PGKMDKGEHRQERRDRPY | Q01844, Q61545 | 202 |
| PKKGDKYDKTD | Q45FA5 | 203 |
| PKKKSRK | O35914, Q01954 | 204 |
| PKKNKPE | Q22663 | 205 |
| PKKRAKV | P04295, P89438 | 206 |
| PKPKKLKVE | P55263, P55262, P55264, Q64640 | 207 |
| PKRGRGR | Q9FYS5, Q43386 | 208 |
| PKRRLVDDA | P0C797 | 209 |
| PKRRRTY | SeqNLS | 210 |
| PLFKRR | A8X6H4, Q9TXJ0 | 211 |
| PLRKAKR | Q86WB0, Q5R8V9 | 212 |
| PPAKRKCIF | Q6AZ28, O75928, Q8C5D8 | 213 |
| PPARRRRL | Q8NAG6 | 214 |
| PPKKKRKV | Q3L6L5, P03070, P14999, P03071 | 215 |
| PPNKRMKVKH | Q8BN78 | 216 |
| PPRIYPQLPSAPT | P0C799 | 217 |
| PQRSPFPKSSVKR | SeqNLS | 218 |
| PRPRKVPR | P0C799 | 219 |
| PRRRVQRKR | SeqNLS, Q5R448, Q5TAQ9 | 220 |
| PRRVRLK | Q58DJ0, P56477, Q13568 | 221 |
| PSRKRPR | Q62315, Q5F363, Q92833 | 222 |
| PSSKKRKV | SeqNLS | 223 |
| PTKKRVK | P07664 | 224 |
| QRPGPYDRP | SeqNLS | 225 |
| RGKGGKGLGKGGAKRHRK | SeqNLS | 226 |
| RKAGKGGGGHKTTKKRSA | B4FG96 | 227 |
| KDEKVP | | |
| RKIKLKRAK | A1L3G9 | 228 |
| RKIKRKRAK | B9X187 | 229 |
| RKKEAPGPREELRSRGR | O35126, P54258, Q5IS70, P54259 | 230 |
| RKKRKGK | SeqNLS, Q29243, Q62165, Q28685, | 231 |
| | O18738, Q9TSZ6, Q14118 | |
| RKKRRQRRR | P04326, P69697, P69698, P05907, | 232 |
| | P20879, P04613, P19553, P0C1J9, | |
| | P20893, P12506, P04612, Q73370, | |
| | P0C1K0, P05906, P35965, P04609, | |
| | P04610, P04614, P04608, P05905 | |
| RKKSIPLSIKNLKRKHKRKK | Q9C0C9 | 233 |
| NKITR | | |
| RKLVKPKNTKMKTKLRTNP | Q14190 | 234 |
| Y | | |
| RKRLILSDKGQLDWKK | SeqNLS, Q91Z62, Q1A730, Q2KHT6, | 235 |
| | Q9CPU7 | |
| RKRLKSK | Q13309 | 236 |
| RKRRVRDNM | Q8QPH4, Q809M7, A8C8X1, Q2VNC5, | 237 |
| | Q38SQ0, 089749, Q6DNQ9, Q809L9, | |
| | Q0A429, Q20NV3, P16509, P16505, | |
| | Q6DNQ5, P16506, Q6XT06, P26118, | |
| | Q2ICQ2, Q2RCG8, Q0A2D0, Q0A2H9, | |
| | Q9IQ46, Q809M3, Q6J847, Q6J856, | |
| | B4URE4, A4GCM7, Q0A440, P26120, | |
| | P16511, | |
| RKRSPKDKKEKDLDGAGKR | Q7RTP6 | 238 |
| RKT | | |
| RKRTPRVDGQTGENDMNK | O94851 | 239 |
| RRRK | | |
| RLPVRRRRRR | P04499, P12541, P03269, P48313, | 240 |
| | P03270 | |
| RLRFRKPKSK | P69469 | 241 |
| RQQRKR | Q14980 | 242 |
| RRDLNSSFETSPKKVK | Q8K3G5 | 243 |
| RRDRAKLR | Q9SLB8 | 244 |
| RRGDGRRR | Q80WE1, Q5R9B4, Q06787, P35922 | 245 |
| RRGRKRKAEKQ | Q812D1, Q5XXA9, Q99JF8, Q8MJG1, | 246 |
| | Q66T72, O75475 | |
| RRKKRR | QOVD86, Q58DS6, Q5R6G2, Q9ERI5, | 247 |
| | Q6AYK2, Q6NYC1 | |
| RRKRSKSEDMDSVESKRRR | Q7TT18 | 248 |
| RRKRSR | Q99PU7, D3ZHS6, Q92560, A2VDM8 | 249 |
| RRPKGKTLQKRKPK | Q6ZN17 | 250 |
| RRRGFERFGPDNMGRKRK | Q63014, Q9DBR0 | 251 |
| RRRGKNKVAAQNCRK | SeqNLS | 252 |
| RRRKRR | Q5FVH8, Q6MZT1, Q08DH5, Q8BQP9 | 253 |
| RRRQKQKGGASRRR | SeqNLS | 254 |
| RRRREGPRARRRR | P08313, P10231 | 255 |
| RRTIRLKLVYDKCDRSCKIQ | SeqNLS | 256 |
| KKNRNKCQYCRFHKCLSVG | | |
| MSHNAIRFGRMPRSEKAKL | | |
| KAE | | |
| RRVPQRKEVSRCRKCRK | Q5RJN4, Q32L09, Q8CAK3, Q9NUL5 | 257 |
| RVGGRRQAVECIEDLLNEP | P03255 | 258 |
| GQPLDLSCKRPRP | | |
| RVVKLRIAP | P52639, Q8JMN0 | 259 |
| RVVRRR | P70278 | 260 |
| SKRKTKISRKTR | Q5RAY1, O00443 | 261 |
| SYVKTVPNRTRTYIKL | P21935 | 262 |
| TGKNEAKKRKIA | P52739, Q8K3J5, Q5RAU9 | 263 |
| TLSPASSPSSVSCPVIPASTD | SeqNLS | 264 |
| ESPGSALNI | | |
| VSKKQRTGKKIH | P52739, Q8K3J5, Q5RAU9 | 265 |
| SPKKKRKVE | | 266 |
| KRTAD GSEFE SPKKKRKVE | | 267 |
| PAAKRVKLD | | 268 |
| PKKKRKV | | 269 |
| MDSLLMNRRKFLYQFKNVR | | 142 |
| WAKGRRETYLC | | |
| SPKKKRKVEAS | | 270 |
| MAPKKKRKVGIHRGVP | | 271 |

In some embodiments, the NLS is a bipartite NLS. A bipartite NLS typically comprises two basic amino acid clusters separated by a spacer sequence (which may be, e.g., about 10 amino acids in length). A monopartite NLS typically lacks a spacer. An example of a bipartite NLS is the nucleoplasmin NLS, having the sequence KR[PAATKKAGQA]KKKK (SEQ ID NO: 272), wherein the spacer is bracketed. Another exemplary bipartite NLS has the sequence PKKKRKVEGADKRTADGSEFESPKKKRKV (SEQ ID NO: 273). Exemplary NLSs are described in International Application WO2020051561, which is herein incorporated by reference in its entirety, including for its disclosures regarding nuclear localization sequences.

### Linkers:

In some embodiments, domains of the compositions and systems described herein (e.g., the endonuclease and reverse transcriptase domains of a polypeptide or the DNA binding domain and reverse transcriptase domains of a polypeptide) may be joined by a linker. A composition described herein comprising a linker element has the general form S1-L-S2, wherein S1 and S2 may be the same or different and represent two domain moieties (e.g., each a polypeptide or nucleic acid domain) associated with one another by the linker. In some embodiments, a linker may connect two polypeptides. In some embodiments, a linker may connect two nucleic acid molecules. In some embodiments, a linker may connect a polypeptide and a nucleic acid molecule. A linker may be a chemical bond, e.g., one or more covalent bonds or non-covalent bonds. A linker may be flexible, rigid, and/or cleavable. In some embodiments, the linker is a peptide linker. Generally, a peptide linker is at least 2, 3, 4, 5, 6, 7, 8, 9, 10 or more amino acids in length, e.g., 2-50 amino acids in length, 2-30 amino acids in length.

The most commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of the linker in aqueous solutions by forming hydrogen bonds with the water molecules, and therefore reduce unfavorable interactions between the linker and the other moieties. Examples of such linkers include those having the structure [GGS]^{≥1} or [GGGS]^{≥1} (SEQ ID NO: 2). Rigid linkers are useful to keep a fixed distance between domains and to maintain their independent functions. Rigid linkers may also be useful when a spatial separation of the domains is critical to preserve the stability or bioactivity of one or more components in the agent. Rigid linkers may have an alpha helix-structure or Pro-rich sequence, (XP)n, with X designating any amino acid, preferably Ala, Lys, or Glu. Cleavable linkers may release free functional domains in vivo. In some embodiments, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between the two Cys residues. In vitro thrombin treatment of CPRSC (SEQ ID NO: 3) results in the cleavage of the thrombin-sensitive sequence, while the reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in compositions described herein may also be carried out by proteases that are expressed in vivo under pathological conditions (e.g. cancer or inflammation), in specific cells or tissues, or constrained within certain cellular compartments. The specificity of many proteases offers slower cleavage of the linker in constrained compartments.

In some embodiments the amino acid linkers are (or are homologous to) the endogenous amino acids that exist between such domains in a native polypeptide. In some embodiments the endogenous amino acids that exist between such domains are substituted but the length is unchanged from the natural length. In some embodiments, additional amino acid residues are added to the naturally existing amino acid residues between domains.

In some embodiments, the amino acid linkers are designed computationally or screened to maximize protein function (Anad et al., FEBS Letters, 587:19, 2013).

In addition to being fully encoded on a single transcript, a polypeptide can be generated by separately expressing two or more polypeptide fragments that reconstitute the holoenzyme. In some embodiments, the Gene Writer polypeptide is generated by expressing as separate subunits that reassemble the holoenzyme through engineered protein-protein interactions. In some embodiments, reconstitution of the holoenzyme does not involve covalent binding between subunits. Peptides may also fuse together through trans-splicing of inteins (Tornabene et al. Sci Transl Med 11, eaav4523 (2019)). In some embodiments, the Gene Writer holoenzyme is expressed as separate subunits that are designed to create a fusion protein through the presence of split inteins (e.g., as described herein) in the subunits. In some embodiments, the Gene Writer holoenzyme is reconstituted through the formation of covalent linkages between subunits. In some embodiments, protein subunits reassemble through engineered protein-protein binding partners, e.g., SpyTag and SpyCatcher (Zakeri et al. PNAS 109, E690-E697 (2012)). In some embodiments, an additional domain described herein, e.g., a Cas9 nickase, is expressed as a separate polypeptide that associates with the Gene Writer polypeptide through covalent or non-covalent interactions as described above. In some embodiments, the breaking up of a Gene Writer polypeptide into subunits may aid in delivery of the protein by keeping the nucleic acid encoding each part within optimal packaging limits of a viral delivery vector, e.g., AAV (Tornabene et al. Sci Transl Med 11, eaav4523 (2019)). In some embodiments, the Gene Writer polypeptide is designed to be dimerized through the use of covalent or non-covalent interactions as described above.

### Inteins

In some embodiments, the Gene Writer system comprises an intein. Generally, an intein comprises a polypeptide that has the capacity to join two polypeptides or polypeptide fragments together via a peptide bond. In some embodiments, the intein is a trans-splicing intein that can join two polypeptide fragments, e.g., to form the polypeptide component of a system as described herein. In some embodiments, an intein may be encoded on the same nucleic acid molecule encoding the two polypeptide fragments. In certain embodiments, the intein may be translated as part of a larger polypeptide comprising, e.g., in order, the first polypeptide fragment, the intein, and the second polypeptide fragment. In embodiments, the translated intein may be capable of excising itself from the larger polypeptide, e.g., resulting in separation of the attached polypeptide fragments. In embodiments, the excised intein may be capable of joining the two polypeptide fragments to each other directly via a peptide bond. Exemplary inteins are described in, e.g., Table X of PCT Application No. PCT/US2021/020943.

### Evolved Variants of Polypeptide Components:

In some embodiments, the invention provides evolved variants of Gene Writers. Evolved variants can, in some embodiments, be produced by mutagenizing a reference Gene Writer, or one of the fragments or domains comprised therein. In some embodiments, one or more of the domains (e.g., a protein domain described herein, e.g., a structural polypeptide, reverse transcriptase, integrase, DNA binding (including, for example, sequence-guided DNA binding elements), or RNA-binding domain) is evolved. One or more of such evolved variant domains can, in some embodiments, be evolved alone or together with other domains, An evolved variant domain or domains may, in some embodiments, be combined with unevolved cognate component(s) or evolved variants of the cognate component(s), e.g., which may have been evolved in either a parallel or serial manner.

In some embodiments, the process of mutagenizing a reference Gene Writer polypeptide, or fragment or domain thereof, comprises mutagenizing the reference Gene Writer polypeptide or fragment or domain thereof. In embodiments, the mutagenesis comprises a continuous evolution method (e.g., PACE) or non-continuous evolution method (e.g., PANCE), e.g., as described herein. In some embodiments, the evolved Gene Writer poly peptide, or a fragment or domain thereof, comprises one or more amino acid variations introduced into its amino acid sequence relative to the amino acid sequence of the reference Gene Writer polypeptide, or fragment or domain thereof. In embodiments, amino acid sequence variations may include one or more mutated residues (e.g., conservative substitutions, non-conservative substitutions, or a combination thereof) within the amino acid sequence of a reference Gene Writer polypeptide, e.g., as a result of a change in the nucleotide sequence encoding the Gene Writer polypeptide that results in, e.g., a change in the codon at any particular position in the coding sequence, the deletion of one or more amino acids (e.g., a truncated protein), the insertion of one or more amino acids, or any combination of the foregoing. The evolved variant Gene Writer polypeptide may include variants in one or more components or domains of the Gene Writer polypeptide (e.g., variants introduced into a domain described herein, e.g., a structural polypeptide, reverse transcriptase, integrase, DNA binding (including, for example, sequence-guided DNA binding elements), or RNA-binding domain, or combinations thereof).

In some aspects, the invention provides Gene Writer genome editors, systems, kits, and methods using or comprising an evolved variant of a Gene Writer polypeptide, e.g., employs an evolved variant of a Gene Writer polypeptide or a Gene Writer poly peptide produced or produceable by PACE. or PANCE. In embodiments, the unevolved reference Gene Writer polypeptide is a Gene Writer polypeptide as disclosed herein.

The term “phage-assisted continuous evolution (PACE),” as used herein, generally refers to continuous evolution that employs phage as viral vectors. Examples of PACE technology have been described, for example, in International PCT Application No. PCT/US 2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012: U.S. Pat. No. 9,023,594, issued May 5, 2015: U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; and International PCT Application, PCT/US2016/027795, filed Apr. 15, 2016, published as WO 2016/168631 on Oct. 20, 2016, the entire contents of each of which are incorporated herein by reference.

The term “phage-assisted non-continuous evolution (PANCE),” as used herein, generally refers to non-continuous evolution that employs phage as viral vectors. Examples of PANCE technology have been described, for example, in Suzuki T. et al., Crystal structures reveal an elusive functional domain of pyrrolysyl-tRNA synthetase, Nat Chen Biol. 13(12): 1261-1266 (2017), incorporated herein by reference in its entirety. Briefly, PANCE is a technique for rapid in vivo directed evolution using serial flask transfers of evolving selection phage (SP), which contain a gene of interest to be evolved, across fresh host cells (e.g., E. coli cells). Genes inside the host cell may be held constant while genes contained in the SP continuously evolve. Following phage growth, an aliquot of infected cells may be used to transfect a subsequent flask containing host E. coli. This process can be repeated and/or continued until the desired phenotype is evolved, e.g., for as many transfers as desired.

Methods of applying PACE and PANCE to Gene Writer components may be readily appreciated by the skilled artisan by reference to, inter alia, the foregoing references. Additional exemplary methods for directing continuous evolution of genome-identifying proteins or systems, e.g., in a population of host cells, e.g., using phage particles, can be applied to generate evolved variants of Gene Writer polypeptides, or fragments or subdomains thereof. Non-limiting examples of such methods are described in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012: U.S. Pat. No. 9,023,594, issued May 5, 2015: U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; international Application No. PCT/US2019/37216, filed Jun. 14, 2019, International Patent Publication WO 2019/023680, published Jan. 31, 2019, International PCT Application, PCT/US2016/027795, filed Apr. 15, 2016, published as WO 2016/168631 on Oct. 20, 2016, and International Patent Publication No. PCT/US2019/47996, filed Aug. 23, 2019, each of which is incorporated herein by reference in its entirety.

In some non-limiting illustrative embodiments, a method of evolution of a evolved variant Gene Writer polypeptide, of a fragment or domain thereof, comprises: (a) contacting a population of host cells with a population of viral vectors comprising the gene of interest (the starting Gene Writer polypeptide or fragment or domain thereof), wherein: (1) the host cell is amenable to infection by the viral vector; (2) the host cell expresses viral genes required for the generation of viral particles; (3) the expression of at least one viral gene required for the production of an infectious viral particle is dependent on a function of the gene of interest; and/or (4) the viral vector allows for expression of the protein in the host cell, and can be replicated and packaged into a viral particle by the host cell. In some embodiments, the method comprises (b) contacting the host cells with a mutagen, using host cells with mutations that elevate mutation rate (e.g., either by carrying a mutation plasmid or some genome modification—e.g., proofing-impaired DNA polymerase, SOS genes, such as UmuC, UmuD′, and/or RecA, which mutations, if plasmid-bound, may be under control of an inducible promoter), or a combination thereof. In some embodiments, the method comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. In some embodiments, the cells are incubated under conditions allowing for the gene of interest to acquire a mutation. In some embodiments, the method further comprises (d) isolating a mutated version of the viral vector, encoding an evolved gene product (e.g., an evolved variant Gene Writer polypeptide, or fragment or domain thereof), from the population of host cells.

The skilled artisan will appreciate a variety of features employable within the above-described framework. For example, in some embodiments, the viral vector or the phage is a filamentous phage, for example, an M13 phage, e.g., an M13 selection phage. In certain embodiments, the gene required for the production of infectious viral particles is the M13 gene III (gIII). In embodiments, the phage may lack a functional gill, but otherwise comprise gI, gII, gIV, gV, gVI, gVII gVIII, gIX, and a gX. In some embodiments, the generation of infectious VSV particles involves the envelope protein VSV-G, Various embodiments can use different retroviral vectors, for example, Murine Leukemia Virus vectors, or Lentiviral vectors. In embodiments, the retroviral vectors can efficiently be packaged with VSV-G envelope protein, e.g., as a substitute for the native envelope protein of the virus.

In some embodiments, host cells are incubated according to a suitable number of viral life cycles, e.g., at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles, which in on illustrative and non-limiting examples of M13 phage is 10-20 minutes per virus life cycle. Similarly, conditions can be modulated to adjust the time a host cell remains in a population of host cells, e.g., about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes. Host cell populations can be controlled in part by density of the host cells, or, in some embodiments, the host cell density in an inflow, e.g., 10³ cells/ml, about 10⁴ cells/ml, about 10⁵ cells/ml, about 5-10⁵ cells/ml, about 10⁶ cells/ml, about 5-10⁶ cells/ml, about 10⁷ cells/ml, about 5-10⁷ cells/ml, about 10⁸ cells/ml, about 5-10⁸ cells/ml, about 10⁹ cells/ml, about 5-10⁹ cells/ml, about 10¹⁰ cells/ml, or about 5-10¹⁰ cells/ml.

### Other Sequence Modifications and Improvements:

In some embodiments, a polypeptide for use in any of the systems described herein can be a molecular reconstruction or ancestral reconstruction based upon the aligned polypeptide sequence of multiple instances, e.g., from sequences representing multiple copies of an LTR retrotransposon in a host genome. In some embodiments, a 5′ or 3′ untranslated region for use in any of the systems described herein can be a molecular reconstruction based upon the aligned 5′ or 3′ untranslated region of multiple retrotransposons. Based on the Accession numbers provided herein, polypeptides or nucleic acid sequences can be aligned, e.g., by using routine sequence analysis tools as Basic Local Alignment Search Tool (BLAST) or CD-Search for conserved domain analysis. Molecular reconstructions can be created based upon sequence consensus, e.g. using approaches described in Ivics et al., Cell 1997, 501-510; Wagstaff et al., Molecular Biology and Evolution 2013, 88-99. In some embodiments, the retrotransposon from which the 5′ or 3′ untranslated region or polypeptide is derived is a young or a recently active mobile element, as assessed via phylogenetic methods such as those described in Boissinot et al., Molecular Biology and Evolution 2000, 915-928.

### Gene Writer System Modifications of DNA Target Sites

In some embodiments, a Gene Writer system is capable of producing an insertion into the target site of at least 45, 50, 55, 60, 65, 70, 75, 80, 85, 90, 95, or 100 nucleotides (and optionally no more than 500, 400, 300, 200, or 100 nucleotides). In some embodiments, a Gene Writer system is capable of producing an insertion into the target site of at least 1, 2, 3, 4, 5, 6, 7, 8, 9, 10, 15, 20, 25, 30, 35, 40, 45, 50, 55, 60, 65, 70, 75, 80, 85, 90, 95, or 100 nucleotides (and optionally no more than 500, 400, 300, 200, or 100 nucleotides). In some embodiments, a Gene Writer system is capable of producing an insertion into the target site of at least 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 kilobases (and optionally no more than 1, 5, 10, or 20 kilobases).

In some embodiments, an insertion as described herein increases or decreases expression (e.g. transcription or translation) of a gene. In some embodiments, an insertion increases or decreases expression (e.g. transcription or translation) of a gene by adding sequences in a promoter or enhancer, e.g. sequences that bind transcription factors. In some embodiments, an insertion alters translation of a gene (e.g. alters an amino acid sequence), inserts or disrupts a start or stop codon, or alters or fixes the translation frame of a gene. In some embodiments, an insertion results in the functional knockout of an endogenous gene by disruption of a coding or regulatory sequence. In some embodiments, an insertion alters splicing of a gene, e.g. by inserting or disrupting a splice acceptor or donor site. In some embodiments, an insertion alters transcript or protein half-life. In some embodiments, an insertion alters protein localization in the cell (e.g. from the cytoplasm to a mitochondria, from the cytoplasm into the extracellular space (e.g. adds a secretion tag)). In some embodiments, an insertion alters (e.g. improves) protein folding (e.g. to prevent accumulation of misfolded proteins). In some embodiments, an insertion alters, increases, or decreases the activity of a gene, e.g., a protein encoded by the gene.

In some embodiments, the GeneWriter polypeptide results in insertion of the heterologous object sequence (e.g., the GFP gene) into the target locus (e.g., rDNA) at an average copy number of at least 0.01, 0.025, 0.05, 0.075, 0.1, 0.15, 0.2, 0.25, 0.3, 0.4, 0.5, 0.75, 1, 1.25, 1.5, 1.75, 2, 2.5, 3, 4, or 5 copies per genome. In some embodiments, a cell described herein (e.g., a cell comprising a heterologous sequence at a target insertion site) comprises the heterologous object sequence at an average copy number of at least 0.01, 0.025, 0.05, 0.075, 0.1, 0.15, 0.2, 0.25, 0.3, 0.4, 0.5, 0.75, 1, 1.25, 1.5, 1.75, 2, 2.5, 3, 4, or 5 copies per genome.

In some embodiments, a system or method described herein results in insertion of the heterologous object sequence only at one target site in the genome of the target cell. Insertion can be measured, e.g., using a threshold of above 1%, 1.5%, 2%, 2.5%, 3%, 3.5%, 4%, 4.5%, 5%, e.g., as described in Example 8 of PCT Application No. PCT/US2019/048607, incorporated herein by reference in its entirety. In some embodiments, a system or method described herein results in insertion of the heterologous object sequence wherein less than 1%, 1.5%, 2%, 2.5%, 3%, 3.5%, 4%, 4.5%, 5%, 10%, 20%, 30%, 40%, or 50% of insertions are at a site other than the target site, e.g., using an assay described herein, e.g., an assay of Example 8 of PCT Application No. PCT/US2019/048607, incorporated herein by reference in its entirety.

In some embodiments, a system or method described herein results in “scarless” insertion of the heterologous object sequence, while in some embodiments, the target site can show deletions or duplications of endogenous DNA as a result of insertion of the heterologous sequence. The mechanisms of different retrotransposons could result in different patterns of duplications or deletions in the host genome occurring during retrotransposition at the target site. In some embodiments, the system results in a scarless insertion, with no duplications or deletions in the surrounding genomic DNA. In some embodiments, the system results in a deletion of less than 1, 2, 3, 4, 5, 10, 50, or 100 bp of genomic DNA upstream of the insertion. In some embodiments, the system results in a deletion of less than 1, 2, 3, 4, 5, 10, 50, or 100 bp of genomic DNA downstream of the insertion. In some embodiments, the system results in a duplication of less than 1, 2, 3, 4, 5, 10, 50, or 100 bp of genomic DNA upstream of the insertion. In some embodiments, the system results in a duplication of less than 1, 2, 3, 4, 5, 10, 50, or 100 bp of genomic DNA downstream of the insertion.

In some embodiments, a system or method described herein results in insertion of a heterologous sequence into a target site in the human genome. In some embodiments, the target site in the human genome has sequence similarity to the corresponding target site of the corresponding wild-type retrotransposase (e.g., the retrotransposase from which the GeneWriter was derived) in the genome of the organism to which it is native. For instance, in some embodiments, the identity between the 40 nucleotides of human genome sequence centered at the insertion site and the 40 nucleotides of native organism genome sequence centered at the insertion site is less than 99.5%, 99%, 98%, 97%, 96%, 95%, 90%, 85%, 80%, 75%, 70%, 60%, or 50%, or is between 50-60%, 60-70%, 70-80%, 80-90%, or 90-100%. In some embodiments, the identity between the 100 nucleotides of human genome sequence centered at the insertion site and the 100 nucleotides of native organism genome sequence centered at the insertion site is less than 99.5%, 99%, 98%, 97%, 96%, 95%, 90%, 85%, 80%, 75%, 70%, 60%, or 50%, or is between 50-60%, 60-70%, 70-80%, 80-90%, or 90-100%. In some embodiments, the identity between the 500 nucleotides of human genome sequence centered at the insertion site and the 500 nucleotides of native organism genome sequence centered at the insertion site is less than 99.5%, 99%, 98%, 97%, 96%, 95%, 90%, 85%, 80%, 75%, 70%, 60%, or 50%, or is between 50-60%, 60-70%, 70-80%, 80-90%, or 90-100%.

In some embodiments, after Gene Writing, the target site surrounding the integrated sequence contains a limited number of insertions or deletions, for example, in less than about 50% or 10% of integration events, e.g., as determined by long-read amplicon sequencing of the target site, e.g., as described in Karst et al. (2020) bioRxiv doi.org/10.1101/645903 (incorporated by reference herein in its entirety). In some embodiments, the target site does not show multiple insertion events, e.g., head-to-tail or head-to-head duplications, e.g., as determined by long-read amplicon sequencing of the target site, e.g., as described in Karst et al. bioRxiv doi.org/10.1101/645903 (2020) (incorporated herein by reference in its entirety). In some embodiments, the target site contains an integrated sequence corresponding to the template RNA. In some embodiments, the target site does not contain insertions resulting from endogenous RNA in more than about 1% or 10% of events, e.g., as determined by long-read amplicon sequencing of the target site, e.g., as described in Karst et al. bioRxiv doi.org/10.1101/645903 (2020) (incorporated herein by reference in its entirety). In some embodiments, the target site contains the integrated sequence corresponding to the template RNA.

In some embodiments, after Gene Writing, the target site contains an integrated sequence corresponding to the template RNA. In embodiments, the target site does not comprise sequence outside of the template RNA (e.g., reverse transcribed endogenous RNA, vector backbone, and/or ITRs), e.g., as determined by long-read amplicon sequencing of the target site (for example, as described in Karst et al. bioRxiv doi.org/10.1101/645903 (2020); incorporated herein by reference in its entirety).

### Applications

By integrating coding genes into a RNA sequence template, the Gene Writer system can address therapeutic needs, for example, by providing expression of a therapeutic transgene in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, the RNA sequence template encodes a promotor region specific to the therapeutic needs of the host cell, for example a tissue specific promotor or enhancer. In still other embodiments, a promotor can be operably linked to a coding sequence.

In embodiments, the Gene Writer™ gene editor system can provide therapeutic transgenes expressing, e.g., replacement blood factors or replacement enzymes, e.g., lysosomal enzymes. For example, the compositions, systems and methods described herein are useful to express, in a target human genome, agalsidase alpha or beta for treatment of Fabry Disease; imiglucerase, taliglucerase alfa, velaglucerase alfa, or alglucerase for Gaucher Disease; sebelipase alpha for lysosomal acid lipase deficiency (Wolman disease/CESD); laronidase, idursulfase, elosulfase alpha, or galsulfase for mucopolysaccharidoses; alglucosidase alpha for Pompe disease. For example, the compositions, systems and methods described herein are useful to express, in a target human genome factor I, II, V, VII, X, XI, XII or XIII for blood factor deficiencies.

In some embodiments, the heterologous object sequence encodes an intracellular protein (e.g., a cytoplasmic protein, a nuclear protein, an organellar protein such as a mitochondrial protein or lysosomal protein, or a membrane protein). In some embodiments, the heterologous object sequence encodes a membrane protein, e.g., a membrane protein other than a CAR, and/or an endogenous human membrane protein. In some embodiments, the heterologous object sequence encodes an extracellular protein. In some embodiments, the heterologous object sequence encodes an enzyme, a structural protein, a signaling protein, a regulatory protein, a transport protein, a sensory protein, a motor protein, a defense protein, or a storage protein. Other exemplary proteins that may be encoded by a heterologous object sequence include, without limitation, a immune receptor protein, e.g. a synthetic immune receptor protein such as a chimeric antigen receptor protein (CAR), a T cell receptor, a B cell receptor, or an antibody.

### Administration

The composition and systems described herein may be used in vitro or in vivo. In some embodiments the system or components of the system are delivered to cells (e.g., mammalian cells, e.g., human cells), e.g., in vitro or in vivo. In some embodiments, the cells are eukaryotic cells, e.g., cells of a multicellular organism, e.g., an animal, e.g., a mammal (e.g., human, swine, bovine) a bird (e.g., poultry, such as chicken, turkey, or duck), or a fish. In some embodiments, the cells are non-human animal cells (e.g., a laboratory animal, a livestock animal, or a companion animal). In some embodiments, the cell is a stem cell (e.g., a hematopoietic stem cell), a fibroblast, or a T cell. In some embodiments, the cell is a non-dividing cell, e.g., a non-dividing fibroblast or non-dividing T cell. In some embodiments, the cell is an HSC and p53 is not upregulated or is upregulated by less than 10%, 5%, 2%, or 1%, e.g., as determined according to the method described in Example 30 of PCT Application No. PCT/US2019/048607, incorporated herein by reference in its entirety. In some embodiments, a Gene Writing system described herein is used to make an edit in HEK293, K562, U2OS, or HeLa cells. In some embodiment, a Gene Writing system is used to make an edit in primary cells, e.g., primary cortical neurons from E18.5 mice. The components of the Gene Writer system may, in some instances, be delivered in the form of polypeptide, nucleic acid (e.g., DNA, RNA), and combinations thereof.

For instance, delivery can use any of the following combinations for delivering one or more retroviral or retrotransposon proteins (e.g., an integrase, structural polypeptide domain, and/or reverse transcriptase polypeptide domain, e.g., as described herein) (e.g., as DNA encoding the retroviral or retrotransposon protein, as RNA encoding the integrase protein, or as the protein itself) and the template RNA (e.g., as DNA encoding the RNA, or as RNA):
1. Retroviral or retrotransposon protein-coding DNA+template DNA
2. Retroviral or retrotransposon protein-coding RNA+template DNA
3. Retroviral or retrotransposon protein-coding DNA+template RNA
4. Retroviral or retrotransposon protein-coding RNA+template RNA
5. Retroviral or retrotransposon protein+template DNA
6. Retroviral or retrotransposon protein+template RNA
7. Retroviral or retrotransposon protein-coding virus+template virus
8. Retroviral or retrotransposon protein-coding virus+template DNA
9. Retroviral or retrotransposon protein-coding virus+template RNA
10. Retroviral or retrotransposon protein-coding DNA+template virus
11. Retroviral or retrotransposon protein-coding RNA+template virus
12. Retroviral or retrotransposon protein+template virus

In some embodiments, the ratio of the construct delivering the retroviral or retrotransposon protein-coding (e.g., a driver construct as described herein) and the template RNA is between 10:1 and 1:10 (e.g., between 10:5 to 10:1, 10:5 to 10:2, 10:5 to 10:1, 5:1 to 2:1, 5:1 to 1:1, 4:1 to 2:1, 4:1 to 1:1, 3:1 to 2:1, 3:1 to 1:1, 2:1 to 1:1, 1:1 to 1:2, 1:1 to 1:3, 1:2 to 1:3, 1:1 to 1:4, 1:2 to 1:4, 1:1 to 1:5, 1:2 to 1:5, 1:1 to 1:10, 1:2 to 1:10, or 1:5 to 1:10). In certain embodiments, the ratio of the construct delivering the retroviral or retrotransposon protein-coding (e.g., a driver construct as described herein) and the template RNA is 1:1.

As indicated above, in some embodiments, the DNA or RNA that encodes the integrase protein is delivered using a virus, and in some embodiments, the template RNA (or the DNA encoding the template RNA) is delivered using a virus.

In some embodiments, a template DNA or RNA does not comprise a sequence encoding a functional viral protein (e.g., gag, pol, or a viral reverse transcriptase and/or integrase as described herein, or functional fragments thereof). In some embodiments, the template DNA or RNA comprises an in-frame deletion of a viral gene, e.g., a gene encoding a functional viral protein (e.g., gag, pol, or a viral reverse transcriptase and/or integrase as described herein, or functional fragments thereof). In some embodiments, the template DNA or RNA is introduced into a cell with (e.g., prior to, concurrently with, or after) a driver construct (e.g., a DNA or RNA driver construct) as described herein (e.g., a driver construct comprising one or more genes encoding functional viral proteins, e.g., gag, pol, or a viral reverse transcriptase and/or integrase as described herein, or functional fragments thereof). In some embodiments, a driver construct has a structure as shown in any of FIGS. 9-13. In some embodiments, a template DNA or RNA has a structure as shown in any of FIGS. 9-13. In some embodiments, the heterologous object sequence is between the first LTR and the second LTR, and one or more sequences encoding functional viral proteins (e.g., gag, pol, or a viral reverse transcriptase and/or integrase as described herein, or functional fragments thereof) is between the first LTR and second LTR (e.g., between the first LTR and the heterologous object sequence).

In some embodiments, a template DNA or RNA comprises one or more sequences encoding a functional viral protein (e.g., gag, pol, or a viral reverse transcriptase and/or integrase as described herein, or functional fragments thereof). In some embodiments, template DNA or RNA comprises a sequence encoding a functional viral gag protein, or a functional fragment thereof. In some embodiments, template DNA or RNA comprises a sequence encoding a functional viral pol protein, or a functional fragment thereof. In some embodiments, template DNA or RNA comprises a sequence encoding a functional viral reverse transcriptase protein, or a functional fragment thereof. In some embodiments, template DNA or RNA comprises a sequence encoding a functional viral integrase protein, or a functional fragment thereof. In certain embodiments, the template DNA or RNA comprises a sequence encoding a functional viral gag protein, a functional viral pol protein, and a functional viral reverse transcriptase and/or integrase protein, e.g., as described herein, or functional fragments thereof. In certain embodiments, the sequences encoding functional viral proteins are positioned between the primer binding site and the heterologous object sequence. In some embodiments, a template DNA or RNA has a structure as shown in any of FIGS. 9-13.

In one embodiments the system and/or components of the system are delivered as nucleic acid. For example, the Gene Writer polypeptide may be delivered in the form of a DNA or RNA encoding the polypeptide, and the template RNA may be delivered in the form of RNA or its complementary DNA to be transcribed into RNA. In some embodiments the system or components of the system are delivered on 1, 2, 3, 4, or more distinct nucleic acid molecules. In some embodiments the system or components of the system are delivered as a combination of DNA and RNA. In some embodiments the system or components of the system are delivered as a combination of DNA and protein. In some embodiments the system or components of the system are delivered as a combination of RNA and protein. In some embodiments the Gene Writer genome editor polypeptide is delivered as a protein.

In some embodiments the system or components of the system are delivered to cells, e.g. mammalian cells or human cells, using a vector. The vector may be, e.g., a plasmid or a virus. In some embodiments delivery is in vivo, in vitro, ex vivo, or in situ. In some embodiments the virus is an adeno associated virus (AAV), a lentivirus, an adenovirus. In some embodiments the system or components of the system are delivered to cells with a viral-like particle or a virosome. In some embodiments the delivery uses more than one virus, viral-like particle or virosome.

In one embodiment, the compositions and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the pharmaceutical compositions described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

Fusosomes interact and fuse with target cells, and thus can be used as delivery vehicles for a variety of molecules. They generally consist of a bilayer of amphipathic lipids enclosing a lumen or cavity and a fusogen that interacts with the amphipathic lipid bilayer. The fusogen component has been shown to be engineerable in order to confer target cell specificity for the fusion and payload delivery, allowing the creation of delivery vehicles with programmable cell specificity (see, for example, the relating to fusosome design, preparation, and usage in PCT Publication No. WO/2020014209, incorporated herein by reference in its entirety).

A Gene Writer system can be introduced into cells, tissues and multicellular organisms. In some embodiments the system or components of the system are delivered to the cells via mechanical means or physical means.

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

### 1.1.1 Tissue Specific Activity/Administration

In some embodiments, a system, template RNA, or polypeptide described herein is administered to or is active in (e.g., is more active in) a target tissue, e.g., a first tissue. In some embodiments, the system, template RNA, or polypeptide is not administered to or is less active in (e.g., not active in) a non-target tissue. In some embodiments, a system, template RNA, or polypeptide described herein is useful for modifying DNA in a target tissue, e.g., a first tissue, (e.g., and not modifying DNA in a non-target tissue).

In some embodiments, a system comprises (a) a polypeptide described herein or a nucleic acid encoding the same, (b) a template nucleic acid (e.g., template RNA) described herein, and (c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the polypeptide.

In some embodiments, the nucleic acid in (b) comprises RNA.

In some embodiments, the nucleic acid in (b) comprises DNA.

In some embodiments, the nucleic acid in (b): (i) is single-stranded or comprises a single-stranded segment, e.g., is single-stranded DNA or comprises a single-stranded segment and one or more double stranded segments; (ii) has inverted terminal repeats; or (iii) both (i) and (ii).

In some embodiments, the nucleic acid in (b) is double-stranded or comprises a double-stranded segment.

In some embodiments, (a) comprises a nucleic acid encoding the polypeptide.

In some embodiments, the nucleic acid in (a) comprises RNA.

In some embodiments, the nucleic acid in (a) comprises DNA.

In some embodiments, the nucleic acid in (a): (i) is single-stranded or comprises a single-stranded segment, e.g., is single-stranded DNA or comprises a single-stranded segment and one or more double stranded segments; (ii) has inverted terminal repeats; or (iii) both (i) and (ii).

In some embodiments, the nucleic acid in (a) is double-stranded or comprises a double-stranded segment.

In some embodiments, the nucleic acid in (a), (b), or (a) and (b) is linear.

In some embodiments, the nucleic acid in (a), (b), or (a) and (b) is circular, e.g., a plasmid or minicircle.

In some embodiments, the heterologous object sequence is in operative association with a first promoter.

In some embodiments, the one or more first tissue-specific expression-control sequences comprises a tissue specific promoter.

In some embodiments, the tissue-specific promoter comprises a first promoter in operative association with: i. the heterologous object sequence, ii. a nucleic acid encoding the transposase, or iii. (i) and (ii).

In some embodiments, the one or more first tissue-specific expression-control sequences comprises a tissue-specific microRNA recognition sequence in operative association with: i. the heterologous object sequence, ii. a nucleic acid encoding the transposase, or iii. (i) and (ii).

In some embodiments, a system comprises a tissue-specific promoter, and the system further comprises one or more tissue-specific microRNA recognition sequences, wherein: i. the tissue specific promoter is in operative association with: I. the heterologous object sequence, II. a nucleic acid encoding the transposase, or III. (I) and (II); and/or ii. the one or more tissue-specific microRNA recognition sequences are in operative association with: I. the heterologous object sequence, II. a nucleic acid encoding the transposase, or III.(I) and (II).

In some embodiments, wherein (a) comprises a nucleic acid encoding the polypeptide, the nucleic acid comprises a promoter in operative association with the nucleic acid encoding the polypeptide.

In some embodiments, the nucleic acid encoding the polypeptide comprises one or more second tissue-specific expression-control sequences specific to the target tissue in operative association with the polypeptide coding sequence.

In some embodiments, the one or more second tissue-specific expression-control sequences comprises a tissue specific promoter.

In some embodiments, the tissue-specific promoter is the promoter in operative association with the nucleic acid encoding the polypeptide.

In some embodiments, the one or more second tissue-specific expression-control sequences comprises a tissue-specific microRNA recognition sequence.

In some embodiments, the promoter in operative association with the nucleic acid encoding the polypeptide is a tissue-specific promoter, the system further comprising one or more tissue-specific microRNA recognition sequences.

In some embodiments, a Gene Writer™ system described herein is delivered to a tissue or cell from the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type. In some embodiments, a Gene Writer™ system described herein is used to treat a disease, such as a cancer, inflammatory disease, infectious disease, genetic defect, or other disease. A cancer can be cancer of the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type, and can include multiple cancers.

In some embodiments, a Gene Writer™ system described herein described herein is administered by enteral administration (e.g. oral, rectal, gastrointestinal, sublingual, sublabial, or buccal administration). In some embodiments, a Gene Writer™ system described herein is administered by parenteral administration (e.g., intravenous, intramuscular, subcutaneous, intradermal, epidural, intracerebral, intracerebroventricular, epicutaneous, nasal, intra-arterial, intra-articular, intracavernous, intraocular, intraosseous infusion, intraperitoneal, intrathecal, intrauterine, intravaginal, intravesical, perivascular, or transmucosal administration). In some embodiments, a Gene Writer™ system described herein is administered by topical administration (e.g., transdermal administration).

In some embodiments, a Gene Writer™ system as described herein can be used to modify an animal cell, plant cell, or fungal cell. In some embodiments, a Gene Writer™ system as described herein can be used to modify a mammalian cell (e.g., a human cell). In some embodiments, a Gene Writer™ system as described herein can be used to modify a cell from a livestock animal (e.g., a cow, horse, sheep, goat, pig, llama, alpaca, camel, yak, chicken, duck, goose, or ostrich). In some embodiments, a Gene Writer™ system as described herein can be used as a laboratory tool or a research tool, or used in a laboratory method or research method, e.g., to modify an animal cell, e.g., a mammalian cell (e.g., a human cell), a plant cell, or a fungal cell.

In some embodiments, a Gene Writer™ system as described herein can be used to express a protein, template, or heterologous object sequence (e.g., in an animal cell, e.g., a mammalian cell (e.g., a human cell), a plant cell, or a fungal cell). In some embodiments, a Gene Writer™ system as described herein can be used to express a protein, template, or heterologous object sequence under the control of an inducible promoter (e.g., a small molecule inducible promoter). In some embodiments, a Gene Writing system or payload thereof is designed for tunable control, e.g., by the use of an inducible promoter. For example, a promoter, e.g., Tet, driving a gene of interest may be silent at integration, but may, in some instances, activated upon exposure to a small molecule inducer, e.g., doxycycline. In some embodiments, the tunable expression allows post-treatment control of a gene (e.g., a therapeutic gene), e.g., permitting a small molecule-dependent dosing effect. In embodiments, the small molecule-dependent dosing effect comprises altering levels of the gene product temporally and/or spatially, e.g., by local administration. In some embodiments, a promoter used in a system described herein may be inducible, e.g., responsive to an endogenous molecule of the host and/or an exogenous small molecule administered thereto.

In some embodiments, a Gene Writing system is used to make changes to non-coding and/or regulatory control regions, e.g., to tune the expression of endogenous genes. In some embodiments, a Gene Writing system is used to induce upregulation or downregulation of gene expression. In some embodiments, a regulatory control region comprises one or more of a promoter, enhancer, UTR, CTCF site, and/or a gene expression control region.

In some embodiments, a Gene Writing system may be used to treat a healthy individual, e.g., as a preventative therapy. Gene Writing systems can, in some embodiments, be targeted to generate mutations, e.g., that have been shown to be protective towards a disease of interest. An exemplary list of such diseases and protective mutation targets can be found in Table 22.

In some embodiments, a nucleic acid component of a system provided by the invention a sequence (e.g., encoding the polypeptide or comprising a heterologous object sequence) is flanked by untranslated regions (UTRs) that modify protein expression levels. Various 5′ and 3′ UTRs can affect protein expression. For example, in some embodiments, the coding sequence may be preceded by a 5′ UTR that modifies RNA stability or protein translation. In some embodiments, the sequence may be followed by a 3′ UTR that modifies RNA stability or translation. In some embodiments, the sequence may be preceded by a 5′ UTR and followed by a 3′ UTR that modify RNA stability or translation. In some embodiments, the 5′ and/or 3′ UTR may be selected from the 5′ and 3′ UTRs of complement factor 3 (C3) (cactcctccccatcctctccctctgtccctctgtccctctgaccctgcactgtcccagcacc (SEQ ID NO: 274)) or orosomucoid 1 (ORM1) (caggacacagccttggatcaggacagagacttgggggccatcctgcccctccaacccgacatgtgtacctcagctttttccctcacttgcat caataaagcttctgtgtttggaacagctaa (SEQ ID NO: 275)) (Asrani et al. RNA Biology 2018). In certain embodiments, the 5′ UTR is the 5′ UTR from C3 and the 3′ UTR is the 3′ UTR from ORM1.

In certain embodiments, a 5′ UTR and 3′ UTR for protein expression, e.g., mRNA (or DNA encoding the RNA) for a Gene Writer polypeptide or heterologous object sequence, comprise optimized expression sequences. In some embodiments, the 5′ UTR comprises GGGAAAUAAGAGAGAAAAGAAGAGUAAGAAGAAAUAUAAGAGCCACC (SEQ ID NO: 132) and/or the 3′ UTR comprising UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 133), e.g., as described in Richner et al. Cell 168(6): P1114-1125 (2017), the sequences of which are incorporated herein by reference.

In some embodiments, a 5′ and/or 3′ UTR may be selected to enhance protein expression. In some embodiments, a 5′ and/or 3′ UTR may be selected to modify protein expression such that overproduction inhibition is minimized. In some embodiments, UTRs are around a coding sequence, e.g., outside the coding sequence and in other embodiments proximal to the coding sequence. In some embodiments additional regulatory elements (e.g., miRNA binding sites, cis-regulatory sites) are included in the UTRs.

In some embodiments, an open reading frame (ORF) of a Gene Writer system, e.g., an ORF of an mRNA (or DNA encoding an mRNA) encoding a Gene Writer polypeptide or one or more ORFs of an mRNA (or DNA encoding an mRNA) of a heterologous object sequence, is flanked by a 5′ and/or 3′ untranslated region (UTR) that enhances the expression thereof. In some embodiments, the 5′ UTR of an mRNA component (or transcript produced from a DNA component) of the system comprises the sequence 5′-GGGAAAUAAGAGAGAAAAGAAGAGUAAGAAGAAAUAUAAGAGCCACC-3′ (SEQ ID NO: 132). In some embodiments, the 3′ UTR of an mRNA component (or transcript produced from a DNA component) of the system comprises the sequence 5′-UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA-3′ (SEQ ID NO: 133). This combination of 5′ UTR and 3′ UTR has been shown to result in desirable expression of an operably linked ORF by Richner et al. Cell 168(6): P1114-1125 (2017), the teachings and sequences of which are incorporated herein by reference. In some embodiments, a system described herein comprises a DNA encoding a transcript, wherein the DNA comprises the corresponding 5′ UTR and 3′ UTR sequences, with T substituting for U in the above-listed sequence). In some embodiments, a DNA vector used to produce an RNA component of the system further comprises a promoter upstream of the 5′ UTR for initiating in vitro transcription, e.g., a T7, T3, or SP6 promoter. The 5′ UTR above begins with GGG, which is a suitable start for optimizing transcription using T7 RNA polymerase. For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

### 1.1.2 Viral Vectors and Components Thereof

Viruses are a useful source of delivery vehicles for the systems described herein, in addition to a source of relevant enzymes or domains as described herein, e.g., as sources of polymerases and polymerase functions used herein, e.g., DNA-dependent DNA polymerase, RNA-dependent RNA polymerase, RNA-dependent DNA polymerase, DNA-dependent RNA polymerase, reverse transcriptase. Some enzymes, e.g., reverse transcriptases, may have multiple activities, e.g., be capable of both RNA-dependent DNA polymerization and DNA-dependent DNA polymerization, e.g., first and second strand synthesis. In some embodiments, the virus used as a Gene Writer delivery system or a source of components thereof may be selected from a group as described by Baltimore Bacteriol Rev 35(3):235-241 (1971).

In some embodiments, the virus is selected from a Group I virus, e.g., is a DNA virus and packages dsDNA into virions. In some embodiments, the Group I virus is selected from, e.g., Adenoviruses, Herpesviruses, Poxviruses.

In some embodiments, the virus is selected from a Group II virus, e.g., is a DNA virus and packages ssDNA into virions. In some embodiments, the Group II virus is selected from, e.g., Parvoviruses. In some embodiments, the parvovirus is a dependoparvovirus, e.g., an adeno-associated virus (AAV).

In some embodiments, the virus is selected from a Group III virus, e.g., is an RNA virus and packages dsRNA into virions. In some embodiments, the Group III virus is selected from, e.g., Reoviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group IV virus, e.g., is an RNA virus and packages ssRNA(+) into virions. In some embodiments, the Group IV virus is selected from, e.g., Coronaviruses, Picornaviruses, Togaviruses. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group V virus, e.g., is an RNA virus and packages ssRNA(−) into virions. In some embodiments, the Group V virus is selected from, e.g., Orthomyxoviruses, Rhabdoviruses. In some embodiments, an RNA virus with an ssRNA(−) genome also carries an enzyme inside the virion that is transduced to host cells with the viral genome, e.g., an RNA-dependent RNA polymerase, capable of copying the ssRNA(−) into ssRNA(+) that can be translated directly by the host.

In some embodiments, the virus is selected from a Group VI virus, e.g., is a retrovirus and packages ssRNA(+) into virions. In some embodiments, the Group VI virus is selected from, e.g., Retroviruses. In some embodiments, the retrovirus is a lentivirus, e.g., HIV-1, HIV-2, SIV, BIV. In some embodiments, the retrovirus is a spumavirus, e.g., a foamy virus, e.g., HFV, SFV, BFV. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps. In some embodiments, the ssRNA(+) is first reverse transcribed and copied to generate a dsDNA genome intermediate from which mRNA can be transcribed in the host cell. In some embodiments, an RNA virus with an ssRNA(+) genome also carries an enzyme inside the virion that is transduced to host cells with the viral genome, e.g., an RNA-dependent DNA polymerase, capable of copying the ssRNA(+) into dsDNA that can be transcribed into mRNA and translated by the host. In some embodiments, the reverse transcriptase from a Group VI retrovirus is incorporated as the reverse transcriptase domain of a Gene Writer polypeptide.

In some embodiments, the virus is selected from a Group VII virus, e.g., is a retrovirus and packages dsRNA into virions. In some embodiments, the Group VII virus is selected from, e.g., Hepadnaviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps. In some embodiments, one or both strands of the dsRNA contained in such virions is first reverse transcribed and copied to generate a dsDNA genome intermediate from which mRNA can be transcribed in the host cell. In some embodiments, an RNA virus with a dsRNA genome also carries an enzyme inside the virion that is transduced to host cells with the viral genome, e.g., an RNA-dependent DNA polymerase, capable of copying the dsRNA into dsDNA that can be transcribed into mRNA and translated by the host. In some embodiments, the reverse transcriptase from a Group VII retrovirus is incorporated as the reverse transcriptase domain of a Gene Writer polypeptide.

In some embodiments, virions used to deliver nucleic acid in this invention may also carry enzymes involved in the process of Gene Writing. For example, a retroviral virion may contain a reverse transcriptase domain that is delivered into a host cell along with the nucleic acid. In some embodiments, an RNA template may be associated with a Gene Writer polypeptide within a virion, such that both are co-delivered to a target cell upon transduction of the nucleic acid from the viral particle. In some embodiments, the nucleic acid in a virion may comprise DNA, e.g., linear ssDNA, linear dsDNA, circular ssDNA, circular dsDNA, minicircle DNA, dbDNA, ceDNA. In some embodiments, the nucleic acid in a virion may comprise RNA, e.g., linear ssRNA, linear dsRNA, circular ssRNA, circular dsRNA. In some embodiments, a viral genome may circularize upon transduction into a host cell, e.g., a linear ssRNA molecule may undergo a covalent linkage to form a circular ssRNA, a linear dsRNA molecule may undergo a covalent linkage to form a circular dsRNA or one or more circular ssRNA. In some embodiments, a viral genome may replicate by rolling circle replication in a host cell. In some embodiments, a viral genome may comprise a single nucleic acid molecule, e.g., comprise a non-segmented genome. In some embodiments, a viral genome may comprise two or more nucleic acid molecules, e.g., comprise a segmented genome. In some embodiments, a nucleic acid in a virion may be associated with one or proteins. In some embodiments, one or more proteins in a virion may be delivered to a host cell upon transduction. In some embodiments, a natural virus may be adapted for nucleic acid delivery by the addition of virion packaging signals to the target nucleic acid, wherein a host cell is used to package the target nucleic acid containing the packaging signals.

In some embodiments, a virion used as a delivery vehicle may comprise a commensal human virus. In some embodiments, a virion used as a delivery vehicle may comprise an anellovirus, the use of which is described in WO2018232017A1, which is incorporated herein by reference in its entirety.

### Adeno-Associated Viruses

In some embodiments, the virus is an adeno-associated virus (AAV). In some embodiments, the AAV genome comprises two genes that encode four replication proteins and three capsid proteins, respectively. In some embodiments, the genes are flanked on either side by 145-bp inverted terminal repeats (ITRs). In some embodiments, the virion comprises up to three capsid proteins (Vp1, Vp2, and/or Vp3), e.g., produced in a 1:1:10 ratio. In some embodiments, the capsid proteins are produced from the same open reading frame and/or from differential splicing (Vp1) and alternative translational start sites (Vp2 and Vp3, respectively). Generally, Vp3 is the most abundant subunit in the virion and participates in receptor recognition at the cell surface defining the tropism of the virus. In some embodiments, Vp1 comprises a phospholipase domain, e.g., which functions in viral infectivity, in the N-terminus of Vp1.

In some embodiments, packaging capacity of the viral vectors limits the size of the base editor that can be packaged into the vector. For example, the packaging capacity of the AAVs can be about 4.5 kb (e.g., about 3.0, 3.5, 4.0, 4.5, 5.0, 5.5, or 6.0 kb), e.g., including one or two inverted terminal repeats (ITRs), e.g., 145 base ITRs.

In some embodiments, recombinant AAV (rAAV) comprises cis-acting 145-bp ITRs flanking vector transgene cassettes, e.g., providing up to 4.5 kb for packaging of foreign DNA. Subsequent to infection, rAAV can, in some instances, express a fusion protein of the invention and persist without integration into the host genome by existing episomally in circular head-to-tail concatemers. rAAV can be used, for example, in vitro and in vivo. In some embodiments, AAV-mediated gene delivery requires that the length of the coding sequence of the gene is equal or greater in size than the wild-type AAV genome.

AAV delivery of genes that exceed this size and/or the use of large physiological regulatory elements can be accomplished, for example, by dividing the protein(s) to be delivered into two or more fragments. In some embodiments, the N-terminal fragment is fused to a split intein-N. In some embodiments, the C-terminal fragment is fused to a split intein-C. In embodiments, the fragments are packaged into two or more AAV vectors.

In some embodiments, dual AAV vectors are generated by splitting a large transgene expression cassette in two separate halves (5 and 3 ends, or head and tail), e.g., wherein each half of the cassette is packaged in a single AAV vector (of <5 kb). The re-assembly of the full-length transgene expression cassette can, in some embodiments, then be achieved upon co-infection of the same cell by both dual AAV vectors. In some embodiments, co-infection is followed by one or more of: (1) homologous recombination (HR) between 5 and 3 genomes (dual AAV overlapping vectors); (2) ITR-mediated tail-to-head concatemerization of 5 and 3 genomes (dual AAV trans-splicing vectors); and/or (3) a combination of these two mechanisms (dual AAV hybrid vectors). In some embodiments, the use of dual AAV vectors in vivo results in the expression of full-length proteins. In some embodiments, the use of the dual AAV vector platform represents an efficient and viable gene transfer strategy for transgenes of greater than about 4.0, 4.1, 4.2, 4.3, 4.4, 4.5, 4.6, 4.7, 4.8, 4.9, or 5.0 kb in size. In some embodiments, AAV vectors can also be used to transduce cells with target nucleic acids, e.g., in the in vitro production of nucleic acids and peptides. In some embodiments, AAV vectors can be used for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994); each of which is incorporated herein by reference in their entirety). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989) (incorporated by reference herein in their entirety).

In some embodiments, a Gene Writer described herein (e.g., with or without one or more guide nucleic acids) can be delivered using AAV, lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as described in U.S. Pat. No. 8,454,972 and as in clinical trials involving AAV. For Adenovirus, the route of administration, formulation and dose can be as described in U.S. Pat. No. 8,404,658 and as in clinical trials involving adenovirus. For plasmid delivery, the route of administration, formulation and dose can be as described in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. In some embodiments, the viral vectors can be injected into the tissue of interest. For cell-type specific Gene Writing, the expression of the Gene Writer and optional guide nucleic acid can, in some embodiments, be driven by a cell-type specific promoter.

In some embodiments, AAV allows for low toxicity, for example, due to the purification method not requiring ultracentrifugation of cell particles that can activate the immune response. In some embodiments, AAV allows low probability of causing insertional mutagenesis, for example, because it does not substantially integrate into the host genome.

In some embodiments, AAV has a packaging limit of about 4.4, 4.5, 4.6, 4.7, or 4.75 kb. In some embodiments, a Gene Writer, promoter, and transcription terminator can fit into a single viral vector. SpCas9 (4.1 kb) may, in some instances, be difficult to package into AAV. Therefore, in some embodiments, a Gene Writer is used that is shorter in length than other Gene Writers or base editors. In some embodiments, the Gene Writers are less than about 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb.

An AAV can be AAV1, AAV2, AAV5 or any combination thereof. In some embodiments, the type of AAV is selected with respect to the cells to be targeted; e.g., AAV serotypes 1, 2, 5 or a hybrid capsid AAV1, AAV2, AAV5 or any combination thereof can be selected for targeting brain or neuronal cells; or AAV4 can be selected for targeting cardiac tissue. In some embodiments, AAV8 is selected for delivery to the liver. Exemplary AAV serotypes as to these cells are described, for example, in Grimm, D. et al., J. Virol. 82: 5887-5911 (2008) (incorporated herein by reference in its entirety). In some embodiments, AAV refers all serotypes, subtypes, and naturally-occurring AAV as well as recombinant AAV. AAV may be used to refer to the virus itself or a derivative thereof. In some embodiments, AAV includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrhlO, AAVLK03, AV10, AAV11, AAV 12, rhlO, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. Additional exemplary AAV serotypes are listed in Table 36.

TABLE 36
Exemplary AAV serotypes.

| Target Tissue | Vehicle | Reference |
|---|---|---|
| Liver | AAV (AAV8¹, AAVrh.8¹, AAVhu.37¹, AAV2/8, AAV2/rh10², | 1. Wang et al., Mol. Ther. 18, 118-25 (2010) |
| | AAV9, AAV2, NP40³, NP59^{2, 3}, AAV3B⁵, AAV-DJ⁴, AAV- | 2. Ginn et al., JHEP Reports, 100065 (2019) |
| | LK01⁴, AAV-LK02⁴, AAV-LK03⁴, AAV-LK19⁴ Adenovirus | 3. Paulk et al., Mol. Ther. 26, 289-303 (2018). |
| | (Ad5, HC-AdV⁶) | 4. L. Lisowski et al., Nature. 506, 382-6 (2014). |
| | | 5. L. Wang et al., Mol. Ther. 23, 1877-87 (2015). |
| | | 6. Hausl Mol Ther (2010) |
| Lung | AAV (AAV4, AAV5, AAV6¹, AAV9, H22²) Adenovirus | 1. Duncan et al., Mol Ther Methods Clin Dev (2018) |
| | (Ad5, Ad3, Ad21, Ad14)³ | 2. Cooney et al., Am J Respir Cell Mol Biol (2019) |
| | | 3. Li et al., Mol Ther Methods Clin Dev (2019) |
| Skin | AAV (AAV6¹, AAV-LK19²) | 1. Petek et al., Mol. Ther. (2010) |
| | | 2. L. Lisowski et al., Nature. 506, 382-6 (2014). |
| HSCs | Adenovirus (HDAd5/35⁺⁺) | Wang et al. Blood Adv (2019) |

In some embodiments, a pharmaceutical composition (e.g., comprising an AAV as described herein) has less than 1000 empty capsids, less than 8% empty capsids, less than 7% empty capsids, less than 500 empty capsids, less than 3% empty capsids, or less than 10% empty capsids. In some embodiments, the pharmaceutical composition has less than about 50% empty capsids. In some embodiments, the number of empty capsids is below the limit of detection. In some embodiments, it is advantageous for the pharmaceutical composition to have low amounts of empty capsids, e.g., because empty capsids may generate an adverse response (e.g., immune response, inflammatory response, liver response, and/or cardiac response), e.g., with little or no substantial therapeutic benefit.

In some embodiments, the residual host cell protein (rHCP) in the pharmaceutical composition is less than or equal to 100 ng/ml rHCP per 1×10¹³ vg/ml, e.g., less than or equal to 40 ng/ml rHCP per 1×10¹³ vg/ml or 1-50 ng/ml rHCP per 1×10¹³ vg/ml. In some embodiments, the pharmaceutical composition comprises less than 10 ng rHCP per 1.0×10¹³ vg, or less than 5 ng rHCP per 1.0×10¹³ vg, less than 4 ng rHCP per 1.0×10¹³ vg, or less than 3 ng rHCP per 1.0×10¹³ vg, or any concentration in between. In some embodiments, the residual host cell DNA (hcDNA) in the pharmaceutical composition is less than or equal to 5×10⁶ pg/ml hcDNA per 1×10¹³ vg/ml, less than or equal to 1.2×10⁶ pg/ml hcDNA per 1×10¹³ vg/ml, or 1×10⁵ pg/ml hcDNA per 1×10¹³ vg/ml. In some embodiments, the residual host cell DNA in said pharmaceutical composition is less than 5.0×10⁵ pg per 1×10¹³ vg, less than 2.0×10⁵ pg per 1.0×10¹³ vg, less than 1.1×10⁵ pg per 1.0×10¹³ vg, less than 1.0×10⁵ pg hcDNA per 1.0×10¹³ vg, less than 0.9×10⁵ pg hcDNA per 1.0×10¹³ vg, less than 0.8×10⁵ pg hcDNA per 1.0×10¹³ vg, or any concentration in between.

In some embodiments, the residual plasmid DNA in the pharmaceutical composition is less than or equal to 1.7×10⁵ pg/ml per 1.0×10¹³ vg/ml, or 1×10⁵ pg/ml per 1×1.0×10¹³ vg/ml, or 1.7×10⁶ pg/ml per 1.0×10¹³ vg/ml. In some embodiments, the residual DNA plasmid in the pharmaceutical composition is less than 10.0×10⁵ pg by 1.0×10¹³ vg, less than 8.0×10⁵ pg by 1.0×10¹³ vg or less than 6.8×10⁵ pg by 1.0×10¹³ vg. In embodiments, the pharmaceutical composition comprises less than 0.5 ng per 1.0×10¹³ vg, less than 0.3 ng per 1.0×10¹³ vg, less than 0.22 ng per 1.0×10¹³ vg or less than 0.2 ng per 1.0×10¹³ vg or any intermediate concentration of bovine serum albumin (BSA). In embodiments, the benzonase in the pharmaceutical composition is less than 0.2 ng by 1.0×10¹³ vg, less than 0.1 ng by 1.0×10¹³ vg, less than 0.09 ng by 1.0×10¹³ vg, less than 0.08 ng by 1.0×10¹³ vg or any intermediate concentration. In embodiments, Poloxamer 188 in the pharmaceutical composition is about 10 to 150 ppm, about 15 to 100 ppm or about 20 to 80 ppm. In embodiments, the cesium in the pharmaceutical composition is less than 50 pg/g (ppm), less than 30 pg/g (ppm) or less than 20 pg/g (ppm) or any intermediate concentration.

In embodiments, the pharmaceutical composition comprises total impurities, e.g., as determined by SDS-PAGE, of less than 10%, less than 8%, less than 7%, less than 6%, less than 5%, less than 4%, less than 3%, less than 2%, or any percentage in between. In embodiments, the total purity, e.g., as determined by SDS-PAGE, is greater than 90%, greater than 92%, greater than 93%, greater than 94%, greater than 95%, greater than 96%, greater than 97%, greater than 98%, or any percentage in between. In embodiments, no single unnamed related impurity, e.g., as measured by SDS-PAGE, is greater than 5%, greater than 4%, greater than 3% or greater than 2%, or any percentage in between. In embodiments, the pharmaceutical composition comprises a percentage of filled capsids relative to total capsids (e.g., peak 1+peak 2 as measured by analytical ultracentrifugation) of greater than 85%, greater than 86%, greater than 87%, greater than 88%, greater than 89%, greater than 90%, greater than 91%, greater than 91.9%, greater than 92%, greater than 93%, or any percentage in between. In embodiments of the pharmaceutical composition, the percentage of filled capsids measured in peak 1 by analytical ultracentrifugation is 20-80%, 25-75%, 30-75%, 35-75%, or 37.4-70.3%. In embodiments of the pharmaceutical composition, the percentage of filled capsids measured in peak 2 by analytical ultracentrifugation is 20-80%, 20-70%, 22-65%, 24-62%, or 24.9-60.1%.

In one embodiment, the pharmaceutical composition comprises a genomic titer of 1.0 to 5.0×10¹³ vg/mL, 1.2 to 3.0×10¹³ vg/mL or 1.7 to 2.3×10¹³ vg/ml. In one embodiment, the pharmaceutical composition exhibits a biological load of less than 5 CFU/mL, less than 4 CFU/mL, less than 3 CFU/mL, less than 2 CFU/mL or less than 1 CFU/mL or any intermediate contraction. In embodiments, the amount of endotoxin according to USP, for example, USP <85> (incorporated by reference in its entirety) is less than 1.0 EU/mL, less than 0.8 EU/mL or less than 0.75 EU/mL. In embodiments, the osmolarity of a pharmaceutical composition according to USP, for example, USP <785> (incorporated by reference in its entirety) is 350 to 450 mOsm/kg, 370 to 440 mOsm/kg or 390 to 430 mOsm/kg. In embodiments, the pharmaceutical composition contains less than 1200 particles that are greater than 25 m per container, less than 1000 particles that are greater than 25 m per container, less than 500 particles that are greater than 25 m per container or any intermediate value. In embodiments, the pharmaceutical composition contains less than 10,000 particles that are greater than 10 m per container, less than 8000 particles that are greater than 10 m per container or less than 600 particles that are greater than 10 pm per container.

In one embodiment, the pharmaceutical composition has a genomic titer of 0.5 to 5.0×10¹³ vg/mL, 1.0 to 4.0×10¹³ vg/mL, 1.5 to 3.0×10¹³ vg/ml or 1.7 to 2.3×10¹³ vg/ml. In one embodiment, the pharmaceutical composition described herein comprises one or more of the following: less than about 0.09 ng benzonase per 1.0×10¹³ vg, less than about 30 pg/g (ppm) of cesium, about 20 to 80 ppm Poloxamer 188, less than about 0.22 ng BSA per 1.0×10¹³ vg, less than about 6.8×10⁵ pg of residual DNA plasmid per 1.0×10¹³ vg, less than about 1.1×10⁵ pg of residual hcDNA per 1.0×10¹³ vg, less than about 4 ng of rHCP per 1.0×10¹³ vg, pH 7.7 to 8.3, about 390 to 430 mOsm/kg, less than about 600 particles that are >25 m in size per container, less than about 6000 particles that are >10 m in size per container, about 1.7×10¹³-2.3×10¹³ vg/mL genomic titer, infectious titer of about 3.9×10⁸ to 8.4×10¹⁰ IU per 1.0×10¹³ vg, total protein of about 100-300 pg per 1.0×10¹³ vg, mean survival of >24 days in A7SMA mice with about 7.5×10¹³ vg/kg dose of viral vector, about 70 to 130% relative potency based on an in vitro cell based assay and/or less than about 5% empty capsid. In various embodiments, the pharmaceutical compositions described herein comprise any of the viral particles discussed here, retain a potency of between ±20%, between +15%, between ±10% or within +5% of a reference standard. In some embodiments, potency is measured using a suitable in vitro cell assay or in vivo animal model.

Additional methods of preparation, characterization, and dosing AAV particles are taught in WO2019094253, which is incorporated herein by reference in its entirety.

Additional rAAV constructs that can be employed consonant with the invention include those described in Wang et al 2019, available at: //doi.org/10.1038/s41573-019-0012-9, including Table 1 thereof, which is incorporated by reference in its entirety.

### 1.1.3 AAV Administration

In some embodiments, an adeno-associated virus (AAV) is used in conjunction with the system, template nucleic acid, and/or polypeptide described herein. In some embodiments, an AAV is used to deliver, administer, or package the system, template nucleic acid, and/or polypeptide described herein. In some embodiments, the AAV is a recombinant AAV (rAAV).

In some embodiments, a system comprises (a) a polypeptide described herein or a nucleic acid encoding the same, (b) a template nucleic acid (e.g., template RNA) described herein, and (c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the polypeptide.

In some embodiments, a system described herein further comprises a first recombinant adeno-associated virus (rAAV) capsid protein; wherein the at least one of (a) or (b) is associated with the first rAAV capsid protein, wherein at least one of (a) or (b) is flanked by AAV inverted terminal repeats (ITRs).

In some embodiments, (a) and (b) are associated with the first rAAV capsid protein.

In some embodiments, (a) and (b) are on a single nucleic acid.

In some embodiments, the system further comprises a second rAAV capsid protein, wherein at least one of (a) or (b) is associated with the second rAAV capsid protein, and wherein the at least one of (a) or (b) associated with the second rAAV capsid protein is different from the at least one of (a) or (b) is associated with the first rAAV capsid protein.

In some embodiments, the at least one of (a) or (b) is associated with the first or second rAAV capsid protein is dispersed in the interior of the first or second rAAV capsid protein, which first or second rAAV capsid protein is in the form of an AAV capsid particle.

In some embodiments, the system further comprises a nanoparticle, wherein the nanoparticle is associated with at least one of (a) or (b).

In some embodiments, (a) and (b), respectively are associated with: a) a first rAAV capsid protein and a second rAAV capsid protein; b) a nanoparticle and a first rAAV capsid protein; c) a first rAAV capsid protein; d) a first adenovirus capsid protein; e) a first nanoparticle and a second nanoparticle; or f) a first nanoparticle.

Viral vectors are useful for delivering all or part of a system provided by the invention, e.g., for use in methods provided by the invention. Systems derived from different viruses have been employed for the delivery of polypeptides, nucleic acids, or transposons; for example: integrase-deficient lentivirus, adenovirus, adeno-associated virus (AAV), herpes simplex virus, and baculovirus (reviewed in Hodge et al. Hum Gene Ther 2017; Narayanavari et al. Crit Rev Biochem Mol Biol 2017; Boehme et al. Curr Gene Ther 2015).

Adenoviruses are common viruses that have been used as gene delivery vehicles given well-defined biology, genetic stability, high transduction efficiency, and ease of large-scale production (see, for example, review by Lee et al. Genes & Diseases 2017). They possess linear dsDNA genomes and come in a variety of serotypes that differ in tissue and cell tropisms. In order to prevent replication of infectious virus in recipient cells, adenovirus genomes used for packaging are deleted of some or all endogenous viral proteins, which are provided in trans in viral production cells. This renders the genomes helper-dependent, meaning they can only be replicated and packaged into viral particles in the presence of the missing components provided by so-called helper functions. A helper-dependent adenovirus system with all viral ORFs removed may be compatible with packaging foreign DNA of up to −37 kb (Parks et al. J Virol 1997). In some embodiments, an adenoviral vector is used to deliver DNA corresponding to the polypeptide or template component of the Gene Writing™ system, or both are contained on separate or the same adenoviral vector. In some embodiments, the adenovirus is a helper-dependent adenovirus (HD-AdV) that is incapable of self-packaging. In some embodiments, the adenovirus is a high-capacity adenovirus (HC-AdV) that has had all or a substantial portion of endogenous viral ORFs deleted, while retaining the necessary sequence components for packaging into adenoviral particles. For this type of vector, the only adenoviral sequences required for genome packaging are noncoding sequences: the inverted terminal repeats (ITRs) at both ends and the packaging signal at the 5′-end (Jager et al. Nat Protoc 2009). In some embodiments, the adenoviral genome also comprises stuffer DNA to meet a minimal genome size for optimal production and stability (see, for example, Hausl et al. Mol Ther 2010). Adenoviruses have been used in the art for the delivery of transposons to various tissues. In some embodiments, an adenovirus is used to deliver a Gene Writing™ system to the liver.

In some embodiments, an adenovirus is used to deliver a Gene Writing™ system to HSCs, e.g., HDAd5/35++. HDAd5/35++ is an adenovirus with modified serotype 35 fibers that de-target the vector from the liver (Wang et al. Blood Adv 2019). In some embodiments, the adenovirus that delivers a Gene Writing™ system to HSCs utilizes a receptor that is expressed specifically on primitive HSCs, e.g., CD46.

Adeno-associated viruses (AAV) belong to the parvoviridae family and more specifically constitute the dependoparvovirus genus. The AAV genome is composed of a linear single-stranded DNA molecule which contains approximately 4.7 kilobases (kb) and consists of two major open reading frames (ORFs) encoding the non-structural Rep (replication) and structural Cap (capsid) proteins. A second ORF within the cap gene was identified that encodes the assembly-activating protein (AAP). The DNAs flanking the AAV coding regions are two cis-acting inverted terminal repeat (ITR) sequences, approximately 145 nucleotides in length, with interrupted palindromic sequences that can be folded into energetically stable hairpin structures that function as primers of DNA replication. In addition to their role in DNA replication, the ITR sequences have been shown to be involved in viral DNA integration into the cellular genome, rescue from the host genome or plasmid, and encapsidation of viral nucleic acid into mature virions (Muzyczka, (1992) Curr. Top. Micro. Immunol. 158:97-129). In some embodiments, one or more Gene Writing™ nucleic acid components is flanked by ITRs derived from AAV for viral packaging. See, e.g., WO2019113310.

In some embodiments, one or more components of the Gene Writing™ system are carried via at least one AAV vector. In some embodiments, the at least one AAV vector is selected for tropism to a particular cell, tissue, organism. In some embodiments, the AAV vector is pseudotyped, e.g., AAV2/8, wherein AAV2 describes the design of the construct but the capsid protein is replaced by that from AAV8. It is understood that any of the described vectors could be pseudotype derivatives, wherein the capsid protein used to package the AAV genome is derived from that of a different AAV serotype. In some embodiments, an AAV to be employed for Gene Writing™ may be evolved for novel cell or tissue tropism as has been demonstrated in the literature (e.g., Davidsson et al. Proc Natl Acad Sci USA 2019).

In some embodiments, the AAV delivery vector is a vector which has two AAV inverted terminal repeats (ITRs) and a nucleotide sequence of interest (for example, a sequence coding for a Gene Writer™ polypeptide or a DNA template, or both), each of said ITRs having an interrupted (or noncontiguous) palindromic sequence, i.e., a sequence composed of three segments: a first segment and a last segment that are identical when read 5′→3′ but hybridize when placed against each other, and a segment that is different that separates the identical segments. Such sequences, notably the ITRs, form hairpin structures. See, for example, WO2012123430.

Conventionally, AAV virions with capsids are produced by introducing a plasmid or plasmids encoding the rAAV or scAAV genome, Rep proteins, and Cap proteins (Grimm et al., 1998). Upon introduction of these helper plasmids in trans, the AAV genome is “rescued” (i.e., released and subsequently recovered) from the host genome, and is further encapsidated to produce infectious AAV. In some embodiments, one or more Gene Writing™ nucleic acids are packaged into AAV particles by introducing the ITR-flanked nucleic acids into a packaging cell in conjunction with the helper functions.

In some embodiments, the AAV genome is a so called self-complementary genome (referred to as scAAV), such that the sequence located between the ITRs contains both the desired nucleic acid sequence (e.g., DNA encoding the Gene Writer™ polypeptide or template, or both) in addition to the reverse complement of the desired nucleic acid sequence, such that these two components can fold over and self-hybridize. In some embodiments, the self-complementary modules are separated by an intervening sequence that permits the DNA to fold back on itself, e.g., forms a stem-loop. An scAAV has the advantage of being poised for transcription upon entering the nucleus, rather than being first dependent on ITR priming and second-strand synthesis to form dsDNA. In some embodiments, one or more Gene Writing™ components is designed as an scAAV, wherein the sequence between the AAV ITRs contains two reverse complementing modules that can self-hybridize to create dsDNA.

In some embodiments, nucleic acid (e.g., encoding a polypeptide, or a template, or both) delivered to cells is closed-ended, linear duplex DNA (CELiD DNA or ceDNA). In some embodiments, ceDNA is derived from the replicative form of the AAV genome (Li et al. PLoS One 2013). In some embodiments, the nucleic acid (e.g., encoding a polypeptide, or a template DNA, or both) is flanked by ITRs, e.g., AAV ITRs, wherein at least one of the ITRs comprises a terminal resolution site and a replication protein binding site (sometimes referred to as a replicative protein binding site). In some embodiments, the ITRs are derived from an adeno-associated virus, e.g., AAV1, AAV2, AAV3, AAV4, AAV5, AAV6, AAV7, AAV8, AAV9, AAV10, AAV 11, AAV12, or a combination thereof. In some embodiments, the ITRs are symmetric. In some embodiments, the ITRs are asymmetric. In some embodiments, at least one Rep protein is provided to enable replication of the construct. In some embodiments, the at least one Rep protein is derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, ceDNA is generated by providing a production cell with (i) DNA flanked by ITRs, e.g., AAV ITRs, and (ii) components required for ITR-dependent replication, e.g., AAV proteins Rep78 and Rep52 (or nucleic acid encoding the proteins). In some embodiments, ceDNA is free of any capsid protein, e.g., is not packaged into an infectious AAV particle. In some embodiments, ceDNA is formulated into LNPs (see, for example, WO2019051289A1). In some embodiments, the ceDNA vector consists of two self complementary sequences, e.g., asymmetrical or symmetrical or substantially symmetrical ITRs as defined herein, flanking said expression cassette, wherein the ceDNA vector is not associated with a capsid protein. In some embodiments, the ceDNA vector comprises two self-complementary sequences found in an AAV genome, where at least one ITR comprises an operative Rep-binding element (RBE) (also sometimes referred to herein as “RBS”) and a terminal resolution site (trs) of AAV or a functional variant of the RBE. See, for example, WO2019113310.

### Inteins

In some embodiments, as described in more detail below, Intein-N may be fused to the N-terminal portion of a first domain described herein, and intein-C may be fused to the C-terminal portion of a second domain described herein for the joining of the N-terminal portion to the C-terminal portion, thereby joining the first and second domains. In some embodiments, the first and second domains are each independent chosen from a DNA binding domain, an RNA binding domain, an RT domain, and an endonuclease domain.

As used herein, “intein” refers to a self-splicing protein intron (e.g., peptide), e.g., which ligates flanking N-terminal and C-terminal exteins (e.g., fragments to be joined). An intein may, in some instances, comprise a fragment of a protein that is able to excise itself and join the remaining fragments (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein (e.g., split intein-N and split intein-C). For example, in cyanobacteria, DnaE, the catalytic subunit a of DNA polymerase III, is encoded by two separate genes, dnaE-n and dnaE-c. The intein encoded by the dnaE-n gene may be herein referred as “intein-N.” The intein encoded by the dnaE-c gene may be herein referred as “intein-C.”

Use of inteins for joining heterologous protein fragments is described, for example, in Wood et al., J. Biol. Chem.289(21); 14512-9 (2014) (incorporated herein by reference in its entirety). For example, when fused to separate protein fragments, the inteins IntN and IntC may recognize each other, splice themselves out, and/or simultaneously ligate the flanking N- and C-terminal exteins of the protein fragments to which they were fused, thereby reconstituting a full-length protein from the two protein fragments.

In some embodiments, a synthetic intein based on the dnaE intein, the Cfa-N (e.g., split intein-N) and Cfa-C(e.g., split intein-C) intein pair, is used. Examples of such inteins have been described, e.g., in Stevens et al., J Am Chem Soc. 2016 Feb. 24; 138(7):2162-5 (incorporated herein by reference in its entirety). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

In some embodiments, Intein-N and intein-C may be fused to the N-terminal portion of the split Cas9 and the C-terminal portion of a split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N [N-terminal portion of the split Cas9]-[intein-N]˜C. In some embodiments, an intein-C is fused to the N-terminus of the C-terminal portion of the split Cas9, i.e., to form a structure of N-[intein-C]˜[C-terminal portion of the split Cas9]-C. The mechanism of intein-mediated protein splicing for joining the proteins the inteins are fused to (e.g., split Cas9) is described in Shah et al., Chem Sci. 2014; 5(1):446-461, incorporated herein by reference. Methods for designing and using inteins are known in the art and described, for example by WO2020051561, WO2014004336, WO2017132580, US20150344549, and US20180127780, each of which is incorporated herein by reference in their entirety.

In some embodiments, a split refers to a division into two or more fragments. In some embodiments, a split Cas9 protein or split Cas9 comprises a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a reconstituted Cas9 protein. In embodiments, the Cas9 protein is divided into two fragments within a disordered region of the protein, e.g., as described in Nishimasu et al., Cell, Volume 156, Issue 5, pp. 935-949, 2014, or as described in Jiang et al. (2016) Science 351: 867-871 and PDB file: 5F9R (each of which is incorporated herein by reference in its entirety). A disordered region may be determined by one or more protein structure determination techniques known in the art, including, without limitation, X-ray crystallography, NMR spectroscopy, electron microscopy (e.g., cryoEM), and/or in silico protein modeling. In some embodiments, the protein is divided into two fragments at any C, T, A, or S, e.g., within a region of SpCas9 between amino acids A292-G364, F445-K483, or E565-T637, or at corresponding positions in any other Cas9, Cas9 variant (e.g., nCas9, dCas9), or other napDNAbp. In some embodiments, protein is divided into two fragments at SpCas9 T310, T313, A456, S469, or C574. In some embodiments, the process of dividing the protein into two fragments is referred to as splitting the protein.

In some embodiments, a protein fragment ranges from about 2-1000 amino acids (e.g., between 2-10, 10-50, 50-100, 100-200, 200-300, 300-400, 400-500, 500-600, 600-700, 700-800, 800-900, or 900-1000 amino acids) in length. In some embodiments, a protein fragment ranges from about 5-500 amino acids (e.g., between 5-10, 10-50, 50-100, 100-200, 200-300, 300-400, or 400-500 amino acids) in length. In some embodiments, a protein fragment ranges from about 20-200 amino acids (e.g., between 20-30, 30-40, 40-50, 50-100, or 100-200 amino acids) in length.

In some embodiments, a portion or fragment of a Gene Writer (e.g., Cas9-R2Tg) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In some embodiments, an endonuclease domain (e.g., a nickase Cas9 domain) is fused to intein-N and a polypeptide comprising an RT domain is fused to an intein-C.

Exemplary nucleotide and amino acid sequences of interns are provided below:

DnaE Intein-N DNA:
(SEQ ID NO: 276)
TGCCTGTCATACGAAACCGAGATACTGACAGTAGAATATGGCCTTCTGCC
AATCGGGAAGATTGTGGAGAAACGGATAGAATGCACAGTTTACTCTGTCG
ATAACAATGGTAACATTTATACTCAGCCAGTTGCCCAGTGGCACGACCGG
GGAGAGCAGGAAGTATTCGAATACTGTCTGGAGGATGGAAGTCTCATTAG
GGCCACTAAGGACCACAAATTTATGACAGTCGATGGCCAGATGCTGCCTA
TAGACGAAATCTTTGAGCGAGAGTTGGACCTCATGCGAGTTGACAACCTT
CCTAAT
DnaE Intein-N Protein:
(SEQ ID NO: 277)
CLSYETEILTVEYGLLPIGKIVEKRIECTVYSVDNNGNIYTQPVAQWHDR
GEQEVFEYCLEDGSLIRATKDHKFMTVDGQMLPIDEIFERELDLMRVDNL
PN
DnaE Intein-C DNA:
(SEQ ID NO: 278)
ATGATCAAGATAGCTACAAGGAAGTATCTTGGCAAACAAAACGTTTATGA
TATTGGAGTCGAAAGAGATCACAACTTTGCTCTGAAGAACGGATTCATAG
CTTCTAAT
Intein-C:
(SEQ ID NO: 279)
MIKIATRKYLGKQNVYDIGVERDHNFALKNGFIASN
Cfa-N DNA:
(SEQ ID NO: 280)
TGCCTGTCTTATGATACCGAGATACTTACCGTTGAATATGGCTTCTTGCC
TATTGGAAAGATTGTCGAAGAGAGAATTGAATGCACAGTATATACTGTAG
ACAAGAATGGTTTCGTTTACACACAGCCCATTGCTCAATGGCACAATCGC
GGCGAACAAGAAGTATTTGAGTACTGTCTCGAGGATGGAAGCATCATACG
AGCAACTAAAGATCATAAATTCATGACCACTGACGGGCAGATGTTGCCAA
TAGATGAGATATTCGAGCGGGGCTTGGATCTCAAACAAGTGGATGGATTG
CCA
Cfa-N Protein:
(SEQ ID NO: 281)
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLKQVDGL
P
Cfa-C DNA:
(SEQ ID NO: 282)
ATGAAGAGGACTGCCGATGGATCAGAGTTTGAATCTCCCAAGAAGAAGAG
GAAAGTAAAGATAATATCTCGAAAAAGTCTTGGTACCCAAAATGTCTATG
ATATTGGAGTGGAGAAAGATCACAACTTCCTTCTCAAGAACGGTCTCGTA
GCCAGCAAC
Cfa-C Protein:
(SEQ ID NO: 283)
MKRTADGSEFESPKKKRKVKIISRKSLGTQNVYDIGVEKDHNFLLKNGLV
ASN

### Lipid Nanoparticles

The methods and systems provided by the invention, may employ any suitable carrier or delivery modality, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference e.g., a lipid-containing nanoparticle can comprise one or more of the lipids in table 4 of WO2019217941. Lipid nanoparticles can include additional elements, such as polymers, such as the polymers described in table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid (e.g., encoding the Gene Writer or template nucleic acid) can be varied as desired. For example, the total lipid to nucleic acid (mass or weight) ratio can be from about 10:1 to about 30:1.

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA) described herein (e.g., a template nucleic acid or a nucleic acid encoding a GeneWriter), encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule, e.g., template RNA and/or a mRNA encoding the Gene Writer polypeptide.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety). In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01) e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is; Imidazole cholesterol ester (ICE) lipid (3S,10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

Some non-limiting example of lipid compounds that may be used (e.g., in combination with other lipid components) to form lipid nanoparticles for the delivery of compositions described herein, e.g., nucleic acid (e.g., RNA) described herein (e.g., a template nucleic acid or a nucleic acid encoding a GeneWriter) includes,

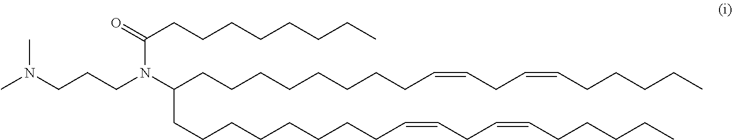 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a GeneWriter composition described herein to the liver and/or hepatocyte cells.

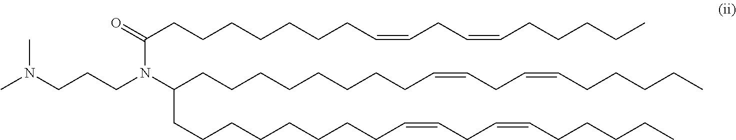 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a GeneWriter composition described herein to the liver and/or hepatocyte cells.

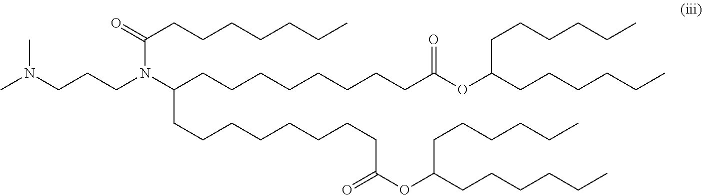 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a GeneWriter composition described herein to the liver and/or hepatocyte cells.

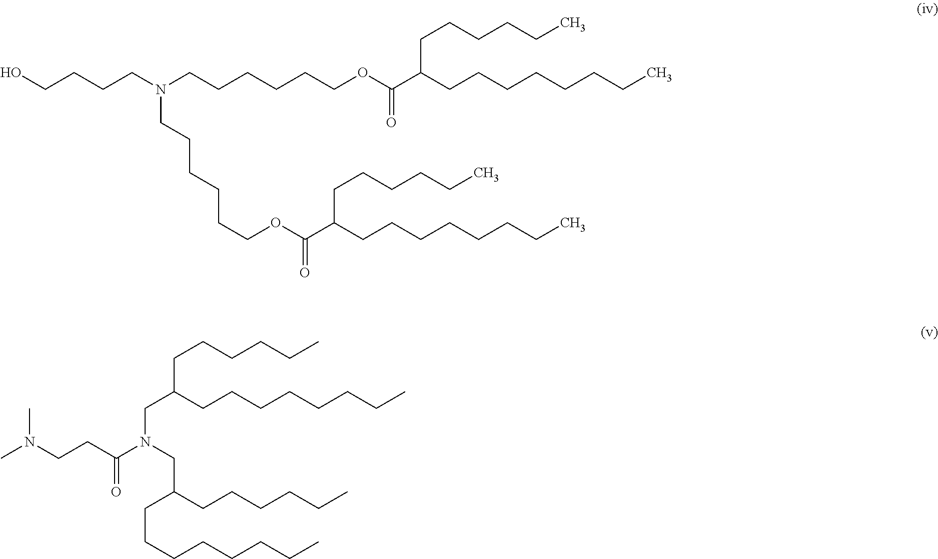

In some embodiments an LNP comprising Formula (v) is used to deliver a GeneWriter composition described herein to the liver and/or hepatocyte cells.

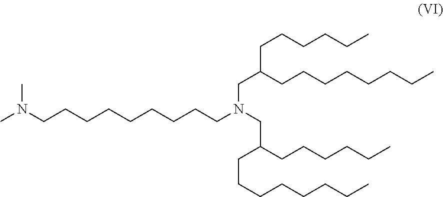 [SMILES: CCCCCCCCC(CCCCCC)CN(CCCCCCCCCN(C)C)CC(CCCCCC)CCCCCCCC]

In some embodiments an LNP comprising Formula (vi) is used to deliver a GeneWriter composition described herein to the liver and/or hepatocyte cells.

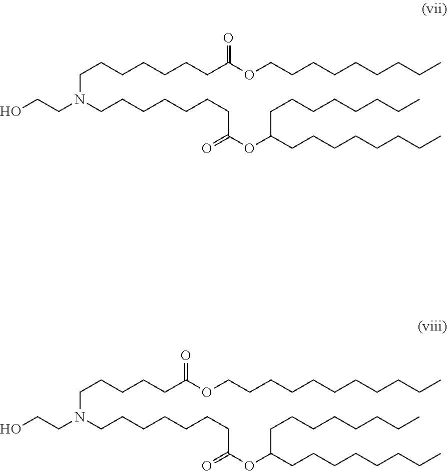 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a GeneWriter composition described herein to the liver and/or hepatocyte cells.

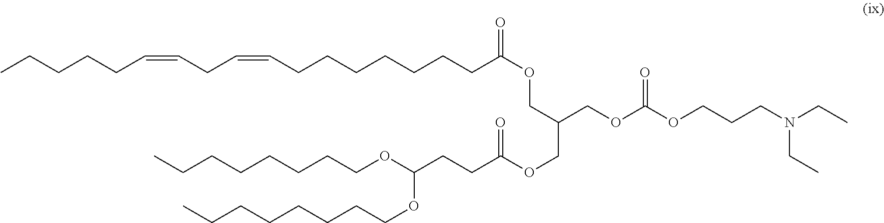 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a GeneWriter composition described herein to the liver and/or hepatocyte cells.

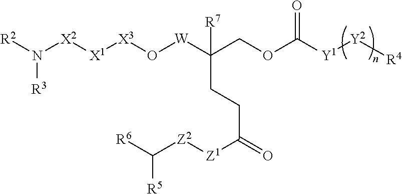

wherein
X¹ is O, NR¹ or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y is selected from

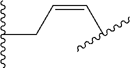 [SMILES: */C=C\C*]

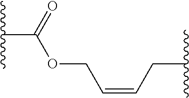 [SMILES: *C/C=C\COC(*)=O]

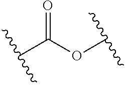 [SMILES: *OC(*)=O]

(in either orientation), (in either orientation), (in either orientation),
n is 0 to 3, Z¹ is Ci-15 alkyl, Z is Ci-6 alkylene or a direct bond,
   Z² is

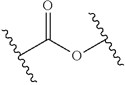 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

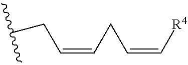

R⁴ is linear C5 alkyl, Z¹ is C2 alkylene, Z is absent, W is methylene, and R⁷ is H, then R⁵ and R⁶ are not Cx alkoxy.

In some embodiments an LNP comprising Formula (xii) is used to deliver a GeneWriter composition described herein to the liver and/or hepatocyte cells.

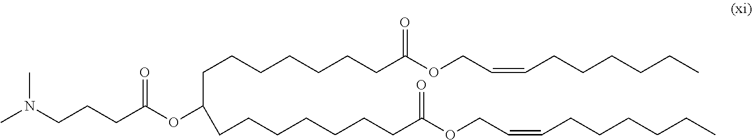 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a GeneWriter composition described herein to the liver and/or hepatocyte cells.

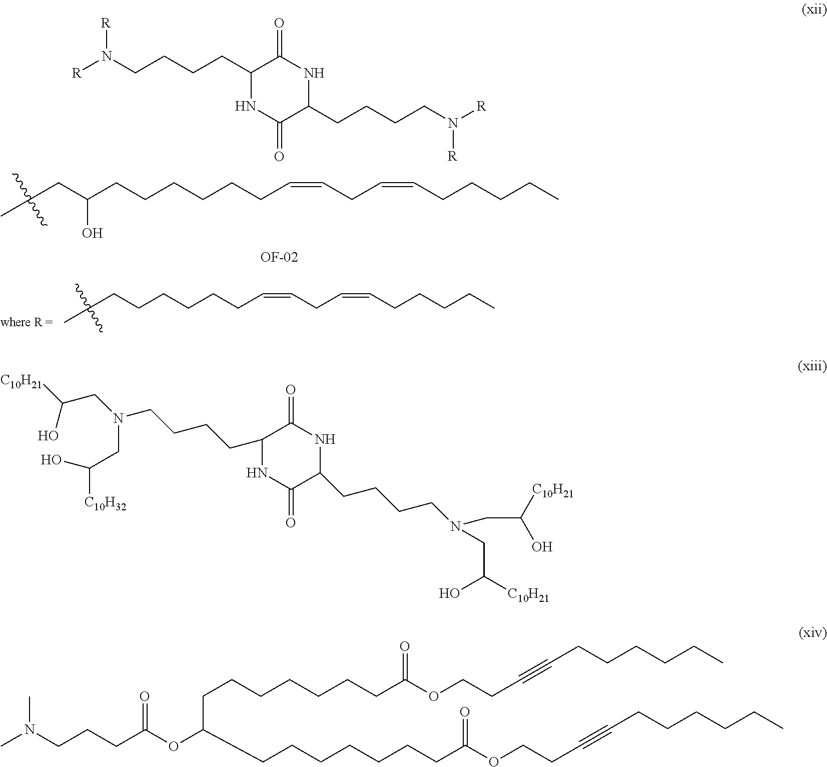

In some embodiments an LNP comprises a compound of Formula (xiii) and a compound of Formula (xiv).

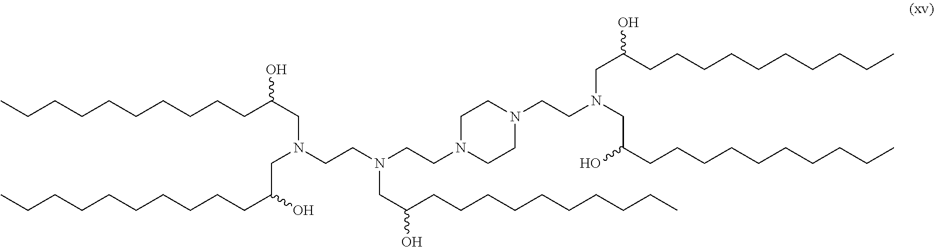 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a GeneWriter composition described herein to the liver and/or hepatocyte cells.

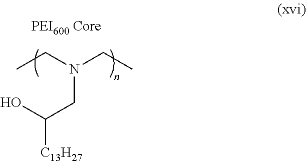

In some embodiments an LNP comprising a formulation of Formula (xvi) is used to deliver a GeneWriter composition described herein to the lung endothelial cells.

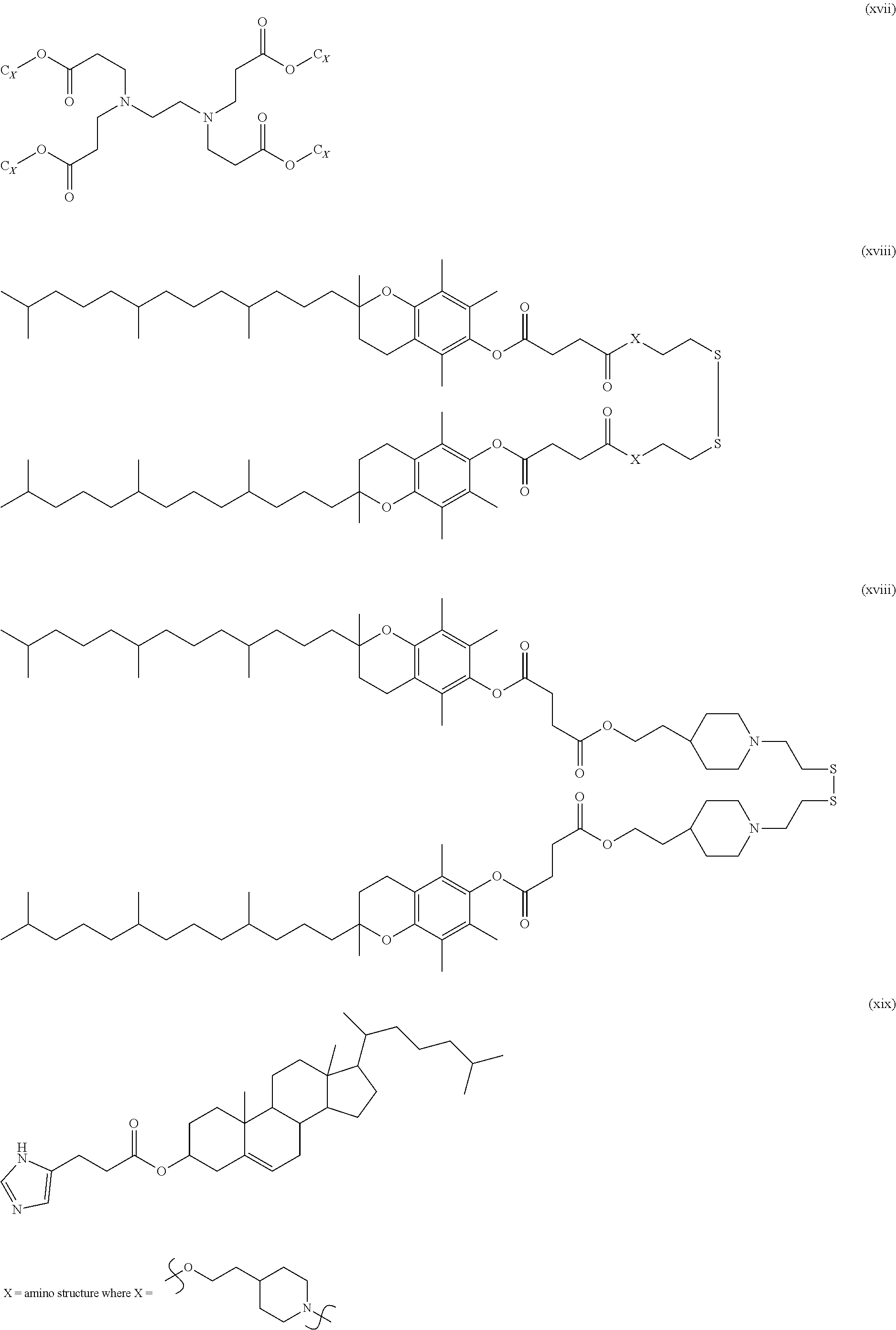

In some embodiments, a lipid compound used to form lipid nanoparticles for the delivery of compositions described herein, e.g., nucleic acid (e.g., RNA) described herein (e.g., a template nucleic acid or a nucleic acid encoding a GeneWriter) is made by one of the following reactions:

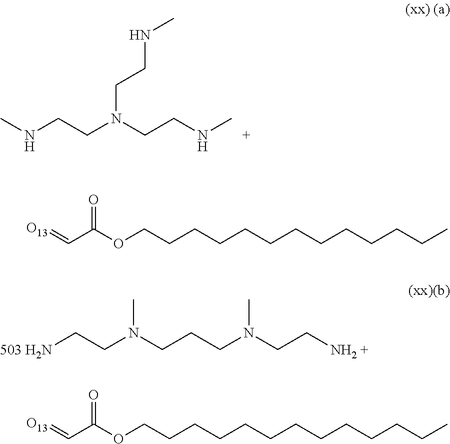

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), 1,2-dioleoyl-sn-glycero-3-phosphoethanolamine (DOPE), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS In some embodiments, the non-cationic lipid may have the following structure,

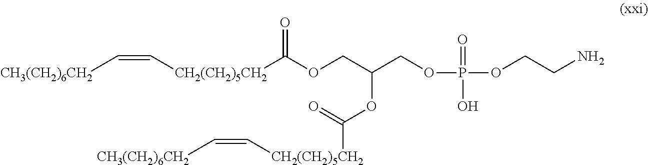

Other examples of non-cationic lipids suitable for use in the lipid nanopartieles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), 1,2-dimyristoyl-sn-glycerol, methoxypoly ethylene glycol (DMG-PEG-2K), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

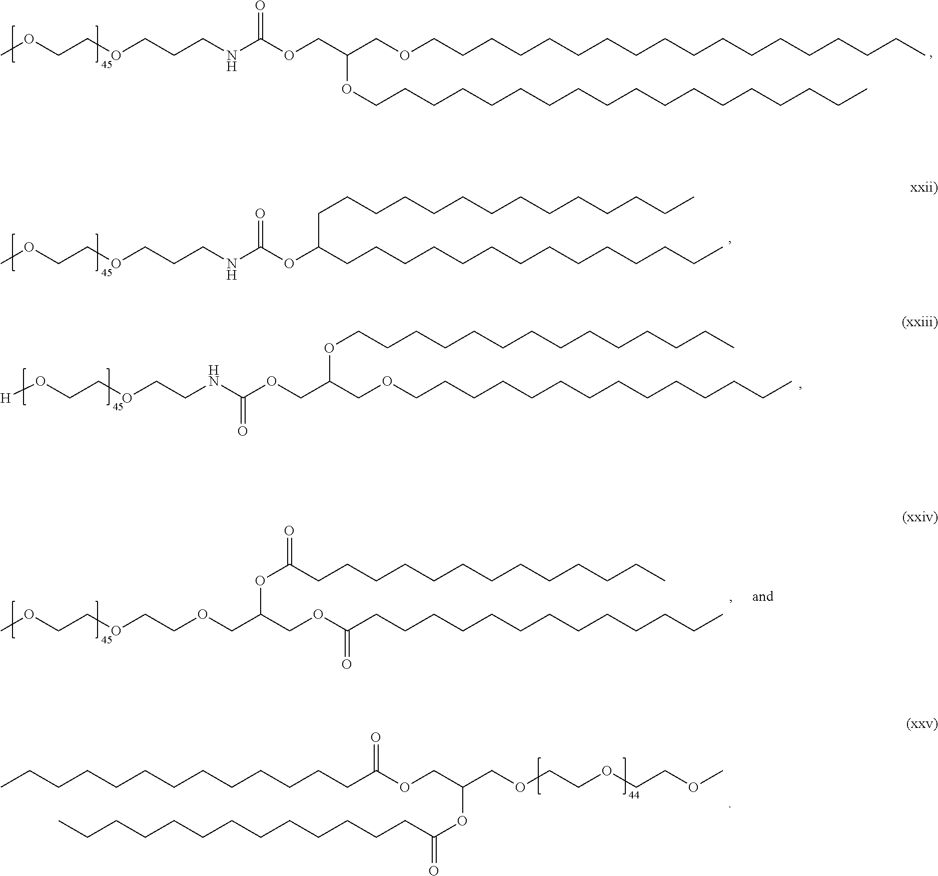

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9 and in WO2020106946A1, the contents of all of which are incorporated herein by reference in their entirety.

In some embodiments an LNP comprises a compound of Formula (xix), a compound of Formula (xxi) and a compound of Formula (xxv). In some embodiments a LNP comprising a formulation of Formula (xix), Formula (xxi) and Formula (xxv) is used to deliver a GeneWriter composition described herein to the lung or pulmonary cells.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, a lipid nanoparticle (or a formulation comprising lipid nanoparticles) lacks reactive impurities (e.g., aldehydes or ketones), or comprises less than a preselected level of reactive impurities (e.g., aldehydes or ketones). While not wishing to be bound by theory, in some embodiments, a lipid reagent is used to make a lipid nanoparticle formulation, and the lipid reagent may comprise a contaminating reactive impurity (e.g., an aldehyde or ketone). A lipid regent may be selected for manufacturing based on having less than a preselected level of reactive impurities (e.g., aldehydes or ketones). Without wishing to be bound by theory, in some embodiments, aldehydes can cause modification and damage of RNA, e.g., cross-linking between bases and/or covalently conjugating lipid to RNA (e.g., forming lipid-RNA adducts). This may, in some instances, lead to failure of a reverse transcriptase reaction and/or incorporation of inappropriate bases, e.g., at the site(s) of lesion(s), e.g., a mutation in a newly synthesized target DNA.

In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, a lipid nanoparticle formulation is produced using a lipid reagent comprising: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation is produced using a plurality of lipid reagents, and each lipid reagent of the plurality independently meets one or more criterion described in this paragraph. In some embodiments, each lipid reagent of the plurality meets the same criterion, e.g., a criterion of this paragraph.

In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation comprises: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, one or more, or optionally all, of the lipid reagents used for a lipid nanoparticle as described herein or a formulation thereof comprise: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, total aldehyde content and/or quantity of any single reactive impurity (e.g., aldehyde) species is determined by liquid chromatography (LC), e.g., coupled with tandem mass spectrometry (MS/MS), e.g., as described herein. In some embodiments, reactive impurity (e.g., aldehyde) content and/or quantity of reactive impurity (e.g., aldehyde) species is determined by detecting one or more chemical modifications of a nucleic acid molecule (e.g., an RNA molecule, e.g., as described herein) associated with the presence of reactive impurities (e.g., aldehydes), e.g., in the lipid reagents. In some embodiments, reactive impurity (e.g., aldehyde) content and/or quantity of reactive impurity (e.g., aldehyde) species is determined by detecting one or more chemical modifications of a nucleotide or nucleoside (e.g., a ribonucleotide or ribonucleoside, e.g., comprised in or isolated from a template nucleic acid, e.g., as described herein) associated with the presence of reactive impurities (e.g., aldehydes), e.g., in the lipid reagents, e.g., as described. In embodiments, chemical modifications of a nucleic acid molecule, nucleotide, or nucleoside are detected by determining the presence of one or more modified nucleotides or nucleosides, e.g., using LC-MS/MS analysis, e.g., as described.

In some embodiments, a nucleic acid (e.g., RNA) described herein (e.g., a template nucleic acid or a nucleic acid encoding a GeneWriter) does not comprise an aldehyde modification, or comprises less than a preselected amount of aldehyde modifications. In some embodiments, on average, a nucleic acid has less than 50, 20, 10, 5, 2, or 1 aldehyde modifications per 1000 nucleotides, e.g., wherein a single cross-linking of two nucleotides is a single aldehyde modification. In some embodiments, the aldehyde modification is an RNA adduct (e.g., a lipid-RNA adduct). In some embodiments, the aldehyde-modified nucleotide is cross-linking between bases. In some embodiments, a nucleic acid (e.g., RNA) described herein comprises less than 50, 20, 10, 5, 2, or 1 cross-links between nucleotide.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, multiple components of a Gene Writer system may be prepared as a single LNP formulation, e.g., an LNP formulation comprises mRNA encoding for the Gene Writer polypeptide and an RNA template. Ratios of nucleic acid components may be varied in order to maximize the properties of a therapeutic. In some embodiments, the ratio of RNA template to mRNA encoding a Gene Writer polypeptide is about 1:1 to 100:1, e.g., about 1:1 to 20:1, about 20:1 to 40:1, about 40:1 to 60:1, about 60:1 to 80:1, or about 80:1 to 100:1, by molar ratio. In other embodiments, a system of multiple nucleic acids may be prepared by separate formulations, e.g., one LNP formulation comprising a template RNA and a second LNP formulation comprising an mRNA encoding a Gene Writer polypeptide. In some embodiments, the system may comprise more than two nucleic acid components formulated into LNPs. In some embodiments, the system may comprise a protein, e.g., a Gene Writer polypeptide, and a template RNA formulated into at least one LNP formulation.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, e.g., Gene Writer polypeptide or mRNA encoding the polypeptide, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of Gene Writer LNP may include about 0.1, 0.25, 0.3, 0.5, 1, 2, 3, 4, 5, 6, 8, 10, or 100 mg/kg (RNA). Exemplary dosing of AAV comprising a nucleic acid encoding one or more components of the system may include an MOI of about 10¹¹, 10¹², 10¹³, and 1014 vg/kg.

### 1.1.3.1 Suitable Indications

Exemplary suitable diseases and disorders that can be treated by the systems or methods provided herein, for example, those comprising Gene Writers, include, without limitation: Baraitser-Winter syndromes 1 and 2; Diabetes mellitus and insipidus with optic atrophy and deafness; Alpha-1-antitrypsin deficiency; Heparin cofactor II deficiency; Adrenoleukodystrophy; Keppen-Lubinsky syndrome; Treacher collins syndrome 1; Mitochondrial complex I, 11, III, III (nuclear type 2, 4, or 8) deficiency; Hypermanganesemia with dystonia, polycythemia and cirrhosis; Carcinoid tumor of intestine; Rhabdoid tumor predisposition syndrome 2; Wilson disease: Hyperphenylalaninemia, bh4-deficient, a, due to partial pts deficiency, BH4-deficient, D, and non-pku; Hyperinsulinemic hypoglycemia familial 3, 4, and 5; Keratosis follicularis; Oral-facial-digital syndrome; SeSAME syndrome; Deafness, nonsyndromic sensorineural, mitochondrial; Proteinuria; Insulin-dependent diabetes mellitus secretory diarrhea syndrome; Moyamoya disease 5; Diamond-Blackfan anemia 1, 5, 8, and 10; Pseudoachondroplastic spondyloepiphyseal dysplasia syndrome; Brittle cornea syndrome 2; Methylmalonic acidemia with homocystinuria, Adams-Oliver syndrome 5 and 6; autosomal recessive Agammaglobulinemia 2, Cortical malformations, occipital, Febrile seizures, familial, 11; Mucopolysaccharidosis type VI, type VI (severe), and type VII; Marden Walker like syndrome, Pseudoneonatal adrenoleukodystrophy; Spheroid body myopathy; Cleidocranial dysostosis; Multiple Cutaneous and Mucosal Venous Malformations; Liver failure acute infantile; Neonatal intrahepatic cholestasis caused by citrin deficiency, Ventricular septal defect 1; Oculodentodigital dysplasia; Wilms tumor I; Weill-Marchesani-like syndrome; Renal adysplasia; Cataract 1, 4, autosomal dominant, autosomal dominant, multiple types, with microcornea, coppock-like, juvenile, with microcomea and glucosuria, and nuclear diffuse nonprogressive; Odontohypophosphatasia; Cerebro-oculo-facio-skeletal syndrome; Schizophrenia 15; Cerebral amyloid angiopathy, APP-related; Hemophagocytic lymphohistiocytosis, familial, 3; Porphobilinogen synthase deficiency; Episodic ataxia type 2; Trichorhinophalangeal syndrome type 3; Progressive familial heart block type IB; Glioma susceptibility I; Lichtenstein-Knorr Syndrome; Hypohidrotic X-linked ectodermal dysplasia; Bartter syndrome types 3, 3 with hypocalciuria, and 4; Carbonic anhydrase VA deficiency, hyperammonemia due to; Cardiomyopathy; Poikiloderma, hereditary fibrosing, with tendon contractures, myopathy, and pulmonary fibrosis; Combined d-2- and 1-2-hydroxyglutaric aciduria; Arginase deficiency, Cone-rod dystrophy 2 and 6; Smith-Lemli-Opitz syndrome, Mucolipidosis III Gamma; Blau syndrome; Weiner syndrome; Meningioma; Iodotyrosyl coupling defect; Dubin-Johnson syndrome; 3-Oxo-5 alpha-steroid delta 4-dehydrogenase deficiency; Boucher Neuhauser syndrome; Iron accumulation in brain; Mental Retardation, X-Linked 102 and syndromic 13; familial, Pituitary adenoma predisposition; Hypoplasia of the corpus callosum; Hyperalphalipoproteinemia 2; Deficiency of ferroxidase; Growth hormone insensitivity with immunodeficiency; Marinesco-Sj\xc3\xb6gren syndrome; Martsolf syndrome; Gaze palsy, familial horizontal, with progressive scoliosis; Mitchell-Riley syndrome; Hypocalciuric hypercalcemia, familial, types 1 and 3; Rubinstein-Taybi syndrome, Epstein syndrome; Juvenile retinoschisis; Becker muscular dystrophy; Loeys-Dietz syndrome 1, 2, 3; Congenital muscular hypertrophy-cerebral syndrome; Familial juvenile gout, Spermatogenic failure 11, 3, and 8; Orofacial cleft 11 and 7. Cleft lip/palate-ectodermal dysplasia syndrome; Mental retardation, X-linked, nonspecific, syndromic, Hedera type, and syndromic, wu type; Combined oxidative phosphorylation deficiencies 1, 3, 4, 12, 15, and 25; Frontotemporal dementia; Kniest dysplasia; Familial cardiomyopathy; Benign familial hematuria; Pheochromocytoma, Aminoglycoside-induced deafness; Gamma-aminobutyric acid transaminase deficiency; Oculocutaneous albinism type IB, type 3, and type 4; Renal coloboma syndrome, CNS hypomyelination; Hennekam lymphangiectasia-lymphedema syndrome 2; Migraine, familial basilar; Distal spinal muscular atrophy, X-linked 3; X-linked periventricular heterotopia, Microcephaly, Mucopolysaccharidosis, MPS-I-H/S, MPS-II, MPS-III-A, MPS-III-B, MPS-III-C, MPS-IV-A, MPS-IV-B; Infantile Parkinsonism-dystonia; Frontotemporal dementia with TDP43 inclusions, TARDBP-related; Hereditary diffuse gastric cancer; Sialidosis type I and II; Microcephaly-capillary malformation syndrome, Hereditary breast and ovarian cancer syndrome; Brain small vessel disease with hemorrhage; Non-ketotic hyperglycinemia; Navajo neurohepatopathy; Auriculocondylar syndrome 2; Spastic paraplegia 15, 2, 3, 35, 39, 4, autosomal dominant, 55, autosomal recessive, and 5A; Autosomal recessive cutis laxa type IA and IB; Hemolytic anemia, nonspherocytic, due to glucose phosphate isomerase deficiency; Hutchinson-Gilford syndrome; Familial amyloid nephropathy with urticaria and deafness: Supravalvar aortic stenosis; Diffuse palmoplantar keratoderma, Bothnian type, Holt-Oram syndrome; Coffin Siris/Intellectual Disability; Left-right axis malformations; Rapadilino syndrome; Nanophthalmos 2; Craniosynostosis and dental anomalies; Paragangliomas 1; Snyder Robinson syndrome; Ventricular fibrillation: Activated PI3K-delta syndrome; Howel-Evans syndrome; Larsen syndrome, dominant type; Van Maldergem syndrome 2; MYH-associated polyposis, 6-pymvoyl-tetrahydropterin synthase deficiency; Alagille syndromes 1 and 2; Lymphangiomyomatosis; Muscle eye brain disease; WFSI-Related Disorders; Primary hypertrophic osteoarthropathy, autosomal recessive 2; Infertility; Nestor-Guillermo progeria syndrome; Mitochondrial trifunctional protein deficiency; Hypoplastic left heart syndrome 2; Primary dilated cardiomyopathy; Retinitis pigmentosa; Hirschsprung disease 3, Upshaw-Schulman syndrome; Desbuquois dysplasia 2; Diarrhea 3 (secretory sodium, congenital, syndromic) and 5 (with tufting enteropathy, congenital); Pachyonychia congenita 4 and type 2, Cerebral autosomal dominant and recessive arteriopathy with subcortical infarcts and leukoencephalopathy, Vi tel Ii form dystrophy; type II, type IV, IV (combined hepatic and myopathic), type V, and type VI; Atypical Rett syndrome; Atrioventricular septal defect 4; Papillon-Lef\xc3\xa8vre syndrome; Leber amaurosis; X-linked hereditary motor and sensory neuropathy; Progressive sclerosing poliodystrophy; Goldmann-Favre syndrome; Renal-hepatic-pancreatic dysplasia; Pallister-Hall syndrome; Amyloidogenic transthyretin amyloidosis; Melnick-Needles syndrome; 1-yperimmunoglobulin E syndrome; Posterior column ataxia with retinitis pigmentosa; Chondrodysplasia punctata 1, X-linked recessive and 2 X-linked dominant; Ectopia lentis, isolated autosomal recessive and dominant; Familial cold urticarial; Familial adenomatous polyposis 1 and 3; Porokeratosis 8, disseminated superficial actinic type; PIK3CA Related Overgrowth Spectrum; Cerebral cavernous malformations 2; Exudative vitreoretinopathy 6; Megalencephaly cutis marmorata telangiectatica congenital; TARP syndrome; Diabetes mellitus, permanent neonatal, with neurologic features; Short-rib thoracic dysplasia 11 or 3 with or without polydactyly; Hypertrichotic osteochondrodysplasia; beta Thalassemia; Niemann-Pick disease type C1, C2, type A, and type C1, adult form; Charcot-Marie-Tooth disease types 1B, 2B2, 2C, 2F, 21, 2U (axonal), 1C (demyelinating), dominant intermediate C. recessive intermediate A, 2A2, 4C, 4D, 4H, IF, IVF, and X; Tyrosinemia type I; Paroxysmal atrial fibrillation; UV-sensitive syndrome; Tooth agenesis, selective, 3 and 4; Merosin deficient congenital muscular dystrophy; Long-chain 3-hydroxyacyl-CoA dehydrogenase deficiency, Congenital aniridia; Left ventricular noncompaction 5; Deficiency of aromatic-L-amino-acid decarboxylase; Coronary heart disease: Leukonychia totalis; Distal arthrogryposis type 2B; Retinitis pigmentosa 10, 11, 12, 14, 15, 17, and 19; Robinow Sorauf syndrome, Tenorio Syndrome; Prolactinoma, Neurofibromatosis, type land type 2; Congenital muscular dystrophy-dystroglycanopathy with brain and eye anomalies, types A2, A7, A8, A1 1, and A14; Heterotaxy, visceral, 2, 4, and 6, autosomal, Jankovic Rivera syndrome, Lipodystrophy, familial partial, type 2 and 3; Hemoglobin H disease, nondeletional; Multicentric osteolysis, nodulosis and arthropathy; Thyroid agenesis; deficiency of Acyl-CoA dehydrogenase family, member 9; Alexander disease; Phytanic acid storage disease; Breast-ovarian cancer, familial 1, 2, and 4; Proline dehydrogenase deficiency; Childhood hypophosphatasia; Pancreatic agenesis and congenital heart disease; Vitamin D-dependent rickets, types land 2; Iridogoniodysgenesis dominant type and type 1, Autosomal recessive hypohidrotic ectodermal dysplasia syndrome; Mental retardation, X-linked, 3, 21, 30, and 72; Hereditary hemorrhagic telangiectasia type 2, Blepharophimosis, ptosis, and epicanthus inversus; Adenine phosphoribosyltransferase deficiency; Seizures, benign familial infantile, 2; Acrodysostosis 2, with or without hormone resistance, Tetralogy of Fallot; Retinitis pigmentosa 2, 20, 25, 35, 36, 38, 39, 4, 40, 43, 45, 48, 66, 7, 70, 72; Lysosomal acid lipase deficiency; Eichsfeld type congenital muscular dystrophy; Walker-Warburg congenital muscular dystrophy; TNF receptor-associated periodic fever syndrome (TRAPS); Progressive myoclonus epilepsy with ataxia; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Long QT syndrome; Dicarboxylic aminoaciduria, Brachydactyly types A1 and A2; Pseudoxanthoma elasticum-like disorder with multiple coagulation factor deficiency; Multisystemic smooth muscle dysfunction syndrome; Syndactyly Cenani Lenz type; Joubert syndrome 1, 6, 7, 9/15 (digenic), 14, 16, and 17, and Orofaciodigital syndrome xiv; Digitorenocerebral syndrome, Retinoblastoma; Dyskinesia, familial, with facial myokymia; Hereditary sensory and autonomic neuropathy type IIB amd IIA; familial hyperinsulinism; Megalencephalic leukoencephalopathy with subcortical cysts land 2a; Aase syndrome; Wiedemann-Steiner syndrome; Ichthvosis exfoliativa; Myotonia congenital; Granulomatous disease, chronic, X-linked, variant; Deficiency of 2-methylbutyryl-CoA dehydrogenase; Sarcoidosis, early-onset; Glaucoma, congenital and Glaucoma, congenital, Coloboma, Breast cancer, susceptibility to, Ceroid lipofuscinosis neuronal 2, 6, 7, and 10; Congenital generalized lipodystrophy type 2; Fructose-biphosphatase deficiency; Congenital contractural arachnodactyly; Lynch syndrome I and II; Phosphoglycerate dehydrogenase deficiency; Burn-Mckeown syndrome, Myocardial infarction 1; Achromatopsia 2 and 7; Retinitis Pigmentosa 73; Protan defect; Polymicrogyria, asymmetric, bilateral frontoparietal; Spinal muscular atrophy, distal, autosomal recessive, 5; Methylmalonic aciduria due to methylmalonyl-CoA mutase deficiency; Familial porencephaly; Hurler syndrome; Oto-palato-digital syndrome, types I and II: Sotos syndrome I or 2; Cardioencephalomyopathy, fatal infantile, due to cytochrome c oxidase deficiency; Parastremmatic dwarfism: Thyrotropin releasing hormone resistance, generalized; Diabetes mellitus, type 2, and insulin-dependent, 20; Thoracic aortic aneurysms and aortic dissections; Estrogen resistance; Maple syrup urine disease type 1A and type 3, Hypospadias 1 and 2, X-linked, Metachromatic leukodystrophy juvenile, late infantile, and adult types: Early T cell progenitor acute lymphoblastic leukemia; Neuropathy, Hereditary Sensory, Type IC; Mental retardation, autosomal dominant 31; Retinitis pigmentosa 39; Breast cancer, early-onset; May-Hegglin anomaly; Gaucher disease type I and Subacute neuronopathic; Temtamy syndrome; Spinal muscular atrophy, lower extremity predominant 2, autosomal dominant; Fanconi anemia, complementation group E, I, N, and O; Alkaptonuria; Hirschsprung disease: Combined malonic and methylmalonic aciduria; Arrhythmogenic right ventricular cardiomyopathy types 5, 8, and 10; Congenital lipomatous overgrowth, vascular malformations, and epidermal nevi; Timothy syndrome; Deficiency of guanidinoacetate methyltransferase; Myoclonic dystonia, Kanzaki disease; Neutral 1 amino acid transport defect; Neurohypophyseal diabetes insipidus: Thyroid hormone metabolism, abnormal; Benign scapuloperoneal muscular dystrophy with cardiomyopathy; Hypoglycemia with deficiency of glycogen synthetase in the liver; Hypertrophic cardiomyopathy: Myasthenic Syndrome, Congenital, 11, associated with acetylcholine receptor deficiency; Mental retardation X-linked syndromic 5; Stormorken syndrome; Aplastic anemia; Intellectual disability; Normokalemic periodic paralysis, potassium-sensitive; Danon disease; Nephronophthisis 13, 15 and 4; Thyrotoxic periodic paralysis and Thyrotoxic periodic paralysis 2, Infertility associated with multi-tailed spermatozoa and excessive DNA; Glaucoma, primary open angle, juvenile-onset; Afibrinogenemia and congenital Afibrinogenemia; Polycystic kidney disease 2, adult type, and infantile type; Familial porphyria cutanea tarda; Cerebello-oculo-renal syndrome (nephronophthisis, oculomotor apraxia and cerebellar abnormalities); Frontotemporal Dementia Chromosome 3-Linked and Frontotemporal dementia ubiquitin-positive; Metatrophic dysplasia: Immunodeficiency-centromeric instability-facial anomalies syndrome 2; Anemia, nonspherocytic hemolytic, due to G6PD deficiency; Bronchiectasis with or without elevated sweat chloride 3; Congenital myopathy with fiber type disproportion; Carney complex, type 1; Cryptorchidism, unilateral or bilateral: Ichthyosis bullosa of Siemens; Isolated lutropin deficiency; DFNA 2 Nonsyndromic Hearing Loss: Klein-Waardenberg syndrome: Gray platelet syndrome; Bile acid synthesis defect, congenital, 2; 46, XY sex reversal, type 1, 3, and 5; Acute intermittent porphyria; Cornelia de Fange syndromes 1 and 5; Hyperglycinuria; Cone-rod dystrophy 3; Dysfibrinogenemia; Karak syndrome, Congenital muscular dystrophy-dystroglycanopathy without mental retardation, type B5; Infantile nystagmus, X-linked; Dyskeratosis congenita, autosomal recessive, 1, 3, 4, and 5; Microcephaly with or without chorioretinopathy, lymphedema, or mental retardation; Hyperlysinemia; Bardet-Biedi syndromes 1, 11, 16, and 19; Autosomal recessive centronuclear myopathy, Frasier syndrome, Caudal regression syndrome; Fibrosis of extraocular muscles, congenital, 1, 2, 3a (with or without extraocular involvement), 3b; Prader-Willi-like syndrome, Malignant melanoma; Bloom syndrome, Darier disease, segmental; Multicentric osteolysis nephropathy; Hemochromatosis type 1, 2B, and 3; Cerebellar ataxia infantile with progressive external ophthalmoplegi and Cerebellar ataxia, mental retardation, and dysequilibrium syndrome 2; Hypoplastic left heart syndrome; Epilepsy, Hearing Loss, And Mental Retardation Syndrome; Transferrin serum level quantitative trait locus 2, Ocular albinism, type 1, Marfan syndrome; Congenital muscular dystrophy-dystroglycanopathy with brain and eye anomalies, type A14 and B14; Hyperammonemia, type III; Cryptophthalmos syndrome, Alopecia universalis congenital; Adult hypophosphatasia; Mannose-binding protein deficiency; Bull eye macular dystrophy; Autosomal dominant torsion dystonia 4; Nephrotic syndrome, type 3, type 5, with or without ocular abnormalities, type 7, and type 9; Seizures, Early infantile epileptic encephalopathy 7; Persistent hyperinsulinemic hypoglycemia of infancy; Thrombocytopenia, X-linked; Neonatal hypotonia; Orstavik Lindemann Solberg syndrome; Pulmonary hypertension, primary, 1, with hereditary hemorrhagic telangiectasia; Pituitary dependent hypercortisolism; Epidermodysplasia vemiciformis; Epidermolysis bullosa, junctional, localisata variant; Cytochrome c oxidase i deficiency; Kindler syndrome; Myosclerosis, autosomal recessive; Truncus arteriosus: Duane syndrome type 2; ADULT syndrome; Zellweger syndrome spectrum; Leukoencephalopathy with ataxia, with Brainstem and Spinal Cord Involvement and Lactate Elevation, with vanishing white matter, and progressive, with ovarian failure: Antithrombin III deficiency; Holoprosencephaly 7; Roberts-SC phocomelia syndrome; Mitochondrial DNA-depletion syndrome 3 and 7, hepatocerebral types, and 13 (encephalomyopathic type); Porencephaly 2; Microcephaly, normal intelligence and immunodeficiency, Giant axonal neuropathy; Sturge-Weber syndrome, Capillary malformations, congenital, 1; Fabry disease and Fabry disease, cardiac variant; Glutamate formiminotransferase deficiency; Fanconi-Bickel syndrome; Acromicric dysplasia; Epilepsy, idiopathic generalized, susceptibility to, 12; Basal ganglia calcification, idiopathic, 4; Polyglucosan body myopathy 1 with or without immunodeficiency; Malignant tumor of prostate, Congenital ectodermal dysplasia of face; Congenital heart disease; Age-related macular degeneration 3, 6, 11, and 12, Congenital myotonia, autosomal dominant and recessive forms, Hypomagnesemia 1, intestinal; Sulfite oxidase deficiency, isolated; Pick disease; Plasminogen deficiency, type 1; Syndactyly type 3, Cone-rod dystrophy amelogenesis imperfecta, Pseudoprimary hyperaldosteronism; Terminal osseous dysplasia; Batter syndrome antenatal type 2, Congenital muscular dystrophy-dystroglycanopathy with mental retardation, types B2, B3, B5, and B15; Familial infantile myasthenia; Lymphoproliferative syndrome 1, 1 (X-linked), and 2; Hypercholesterolaemia and Hypercholesterolemia, autosomal recessive; Neoplasm of ovary; Infantile GM1 gangliosidosis; Syndromic X-linked mental retardation 16; Deficiency of ribose-5-phosphate isomerase; Alzheimer disease, types, 1, 3, and 4; Andersen Tawil syndrome; Multiple synostoses syndrome 3; Chilbain lupus L; Hemophagocytic lymphohistiocytosis, familial, 2; Axenfeld-Rieger syndrome type 3; Myopathy, congenital with cores; Osteoarthritis with mild chondrodysplasia; Peroxisome biogenesis disorders; Severe congenital neutropenia; Hereditary neuralgic amyotrophy; Palmoplantar keratoderma, nonepidermolytic, focal or diffuse; Dysplasminogenemia; Familial colorectal cancer; Spastic ataxia 5, autosomal recessive, Charlevoix-Saguenay type, 1,10, or 11, autosomal recessive; Frontometaphyseal dysplasia land 3; Hereditary factors II, IX, VIII deficiency disease; Spondylocheirodysplasia, Ehlers-Danlos syndrome-like, with immune dysregulation, Aggrecan type, with congenital joint dislocations, short limb-hand type, Sedaghatian type, with cone-rod dystrophy, and Kozlowski type; Ichlhyosis prematurity syndrome, Stickler syndrome type 1, Focal segmental glomerulosclerosis 5; 5-Oxoprolinase deficiency; Microphthalmia syndromic 5, 7, and 9; Juvenile polyposis/hereditary hemorrhagic telangiectasia syndrome; Deficiency of butyryl-CoA dehydrogenase; Maturity-onset diabetes of the young, type 2; Mental retardation, syndromic. Claes-Jensen type, X-linked; Deafness, cochlear, with myopia and intellectual impairment, without vestibular involvement, autosomal dominant, X-linked 2; Spondylocarpotarsal synostosis syndrome; Sting-associated vasculopathy, infantile-onset; Neutral lipid storage disease with myopathy; Immune dysfunction with T-cell inactivation due to calcium entry defect 2; Cardiofaciocutaneous syndrome; Corticosterone methyloxidase type 2 deficiency; Hereditary myopathy with early respiratory failure; Interstitial nephritis, karyomegalic; Trimethylaminuria; Hyperimmunoglobulin D with periodic fever; Malignant hyperthermia susceptibility type 1; Trichomegaly with mental retardation, dwarfism and pigmentary degeneration of retina: Breast adenocarcinoma; Complement factor B deficiency; Ullrich congenital muscular dystrophy; Left ventricular noncompaction cardiomyopathy; Fish-eye disease; Finnish congenital nephrotic syndrome; Limb-girdle muscular dystrophy, type IB, 2A, 2B, 2D, C1, C5, C9, C14; Idiopathic fibrosing alveolitis, chronic form; Primary familial hypertrophic cardiomyopathy; Angiotensin i-converting enzyme, benign serum increase; Cd8 deficiency, familial; Proteus syndrome; Glucose-6-phosphate transport defect; Borjeson-Forssman-Lehmann syndrome; Zellweger syndrome, Spinal muscular atrophy, type II, Prostate cancer, hereditary, 2, Thrombocytopenia, platelet dysfunction. hemolysis, and imbalanced globin synthesis: Congenital disorder of glycosylation types IB, ID, 1G, 1H, 1J, 1K, IN, IP, 2C, 2J, 2K, llm; Junctional epidermolysis bullosa gravis of Herlitz; Generalized epilepsy with febrile seizures plus 3, type 1, type 2; Schizophrenia 4, Coronary artery disease, autosomal dominant 2; Dyskeratosis congenita, autosomal dominant, 2 and 5; Subcortical laminar heterotopia, X-linked; Adenylate kinase deficiency, X-linked severe combined immunodeficiency; Coproporphyria; Amyloid Cardiomyopathy, Transthyretin-related; Hypocalcemia, autosomal dominant 1; Brugada syndrome; Congenital myasthenic syndrome, acetazolamide-responsive; Primary hypomagnesemia; Sclerosteosis, Frontotemporal dementia and/or amyotrophic lateral sclerosis 3 and 4; Mevalonic aciduria; Schwannomatosis 2; Hereditary motor and sensory neuropathy with optic atrophy, Porphyria cutanea tarda; Osteochondritis dissecans; Seizures, benign familial neonatal, 1, and/or myokymia; Long QT syndrome, LQT1 subtype; Mental retardation, anterior maxillary protrusion, and strabismus, Idiopathic hypercalcemia of infancy; Hypogonadotropic hypogonadism 11 with or without anosmia; Polycystic lipomembranous osteodysplasia with sclerosing leukoencephalopathy, Primary autosomal recessive microcephaly 10, 2, 3, and 5; Interrupted aortic arch; Congenital amegakarvocytic thrombocytopenia; Hermansky-Pudlak syndrome 1, 3, 4, and 6; Long QT syndrome 1, 2, 2/9, 2/5, (digenic), 3, 5 and 5, acquired, susceptibility to; Andermann syndrome; Retinal cone dystrophy 3B; Erythropoietic protoporphyria; Sepiapterin reductase deficiency; Very long chain acyl-CoA dehydrogenase deficiency, Hyperferritinemia cataract syndrome; Silver spastic paraplegia syndrome; Charcot-Marie-Tooth disease; Atrial septal defect 2; Carnevale syndrome; Hereditary insensitivity to pain with anhidrosis; Catecholaminergic polymorphic ventricular tachycardia; Hypokalemic periodic paralysis 1 and 2; Sudden infant death syndrome; Hypochromic microcytic anemia with iron overload; GLUT deficiency syndrome 2; Leukodystrophy, Hypomyelinating, II and 6; Cone monochromatism; Osteopetrosis autosomal dominant type I and 2, recessive 4, recessive 1, recessive 6, Severe congenital neutropenia 3, autosomal recessive or dominant, Methionine adenosyltransferase deficiency, autosomal dominant; Paroxysmal familial ventricular fibrillation; Pyruvate kinase deficiency of red cells; Schneckenbecken dysplasia; Torsades de pointes; Distal myopathy Markesbery-Griggs type; Deficiency of UDPglucose-hexose-1-phosphate uridylyltransferase; Sudden cardiac death, Neu-Laxova syndrome 1; Atransferrinemia; Hyperparathyroidism 1 and 2; Cutaneous malignant melanoma 1; Symphalangism, proximal, lb; Progressive pseudorheumatoid dysplasia; Werdnig-Hoffmann disease; Achondrogenesis type 2; Holoprosencephaly 2, 3,7, and 9, Schindler disease, type 1; Cerebroretinal microangiopathy with calcifications and cysts; Heterotaxy, visceral, X-linked; Tuberous sclerosis syndrome; Kartagener syndrome; Thyroid hormone resistance, generalized, autosomal dominant; Bestrophinopathy, autosomal recessive; Nail disorder, nonsyndromic congenital, 8; Mohr-Tranebjaerg syndrome; Cone-rod dystrophy 12; Hearing impairment; Ovarioleukodystrophy, Renal tubular acidosis, proximal, with ocular abnormalities and mental retardation; Dihydropteridine reductase deficiency; Focal epilepsy with speech disorder with or without mental retardation, Ataxia-telangiectasia syndrome, Brown-Vialetto-Van laere syndrome and Brown-Vialetto-Van Laere syndrome 2; Cardiomyopathy; Peripheral demyelinating neuropathy, central dysmyelination, Comeal dystrophy, Fuchs endothelial, 4, Cowden syndrome 3, Dystonia 2 (torsion, autosomal recessive), 3 (torsion, X-linked), 5 (Dopa-responsive type), 10, 12, 16, 25, 26 (Myoclonic); Epiphyseal dysplasia, multiple, with myopia and conductive deafness; Cardiac conduction defect, nonspecific; Branchiootic syndromes 2 and 3; Peroxisome biogenesis disorder 14B, 2A, 4A, 5B, 6A, 7A, and 7B; Familial renal glucosuria; Candidiasis, familial, 2, 5, 6, and 8; Autoimmune disease, multisystem, infantile-onset; Early infantile epileptic encephalopathy 2, 4, 7, 9, 10, 11, 13, and 14; Segawa syndrome, autosomal recessive; Deafness, autosomal dominant 3a, 4, 12, 13, 15, autosomal dominant nonsyndromic sensorineural 17, 20, and 65, Congenital dyserythropoietic anemia, type I and II; Enhanced s-cone syndrome; Adult neuronal ceroid lipofuscinosis; Atrial fibrillation, familial, 11, 12, 13, and 16; Norum disease; Osteosarcoma; Partial albinism; Biotinidase deficiency; Combined cellular and humoral immune defects with granulomas; Alpers encephalopathy; Holocarboxylase synthetase deficiency; Maturity-onset diabetes of the young, type 1, type 2, type 11, type 3, and type 9; Variegate porphyria; Infantile cortical hyperostosis; Testosterone 17-beta-dehydrogenase deficiency; L-2-hydroxyglutaric aciduria; Tyrosinase-negative oculocutaneous albinism; Primary ciliary dyskinesia 24; Pontocerebellar hypoplasia type 4; Ciliary dyskinesia, primary, 7, 11, 15, 20 and 22; Idiopathic basal ganglia calcification 5; Brain atrophy; Craniosynostosis 1 and 4; Keratoconus 1: Rasopathy; Congenital adrenal hyperplasia and Congenital adrenal hypoplasia, X-linked, Mitochondrial DNA depletion syndrome 11, 12 (cardiomyopathic type), 2, 4B (MNGIE type), 8B (MNGIE type); Brachydactyly with hypertension; Cornea plana 2; Aarskog syndrome; Multiple epiphyseal dysplasia 5 or Dominant, Comeal endothelial dystrophy type 2; Aminoacylase 1 deficiency; Delayed speech and language development; Nicolaides-Baraitser syndrome; Enterokinase deficiency, Ectrodactyly, ectodermal dysplasia, and cleft lip/palate syndrome 3; Arthrogryposis multiplex congenita, distal, X-linked; Perrault syndrome 4; Jervell and Lange-Nielsen syndrome 2; Hereditary Nonpolyposis Colorectal Neoplasms, Robinow syndrome, autosomal recessive, autosomal recessive, with brachy-syn-polydactyly: Neurofibrosarcoma; Cytochrome-c oxidase deficiency; Vesicoureteral reflux 8; Dopamine beta hydroxylase deficiency; Carbohydrate-deficient glycoprotein syndrome type I and II; Progressive familial intrahepatic cholestasis 3; Benign familial neonatal-infantile seizures; Pancreatitis, chronic, susceptibility to, Rhizomelic chondrodysplasia punctata type 2 and type 3; Disordered steroidogenesis due to cytochrome p450 oxidoreductase deficiency; Deafness with labyrinthine aplasia microtia and microdontia (FAMM); Rothmund-Thomson syndrome; Cortical dysplasia, complex, with other brain malformations 5 and 6; Myasthenia, familial infantile. 1; Trichorhinophalangeal dysplasia type I; Worth disease, Splenic hypoplasia; Molybdenum cofactor deficiency, complementation group A; Sebastian syndrome: Progressive familial intrahepatic cholestasis 2 and 3, Weill-Marchesani syndrome 1 and 3; Microcephalic osteodysplastic primordial dwarfism type 2; Surfactant metabolism dysfunction, pulmonary, 2 and 3; Severe X-linked myotubular myopathy; Pancreatic cancer 3; Platelet-type bleeding disorder 15 and 8, Tyrosinase-positive oculocutaneous albinism, Borrone Di Rocco Crovato syndrome; ATR-X syndrome; Sucrase-isomaltase deficiency; Complement component 4, partial deficiency of, due to dysfunctional cl inhibitor, Congenital central hypoventilation; Infantile hypophosphatasia; Plasminogen activator inhibitor type 1 deficiency; Malignant lymphoma, non-Hodgkin; Hyperornithinemia-hyperammonemia-homocitrullinuria syndrome; Schwartz Jampel syndrome type 1; Fetal hemoglobin quantitative trait locus 1; Myopathy, distal, with anterior tibial onset; Noonan syndrome 1 and 4, LEOPARD syndrome 1; Glaucoma 1, open angle, e, F, and G; Kenny-Caffey syndrome type 2; PTEN hamartoma tumor syndrome; Duchenne muscular dystrophy; Insulin-resistant diabetes mellitus and acanthosis nigricans; Microphthalmia, isolated 3, 5, 6, 8, and with coloboma 6; Raine syndrome; Premature ovarian failure 4, 5, 7, and 9; Allan-Hemdon-Dudley syndrome; Citrullinemia type I; Alzheimer disease, familial, 3, with spastic paraparesis and apraxia; Familial hemiplegic migraine types I and 2; Ventriculomegaly with cystic kidney disease; Pseudoxanthoma elasticum; Homocysteinemia due to MTHFR deficiency, CBS deficiency, and Homocystinuria, pyridoxine-responsive; Dilated cardiomyopathy 1A, 1AA, 1C, 1G, IBB, 1DD, IFF, 1HH, II, IKK, IN, IS, 1Y, and 3B; Muscle AMP guanine oxidase deficiency, Familial cancer of breast; Hereditary sideroblastic anemia, Myoglobinuria, acute recurrent, autosomal recessive; Neuroferritinopathy; Cardiac arrhythmia; Glucose transporter type 1 deficiency syndrome; Holoprosencephaly sequence, Angiopathy, hereditary, with nephropathy, aneurysms, and muscle cramps; Isovaleryl-CoA dehydrogenase deficiency; Kallmann syndrome 1, 2, and 6; Permanent neonatal diabetes mellitus, Acrocallosal syndrome, Schinzel type; Gordon syndrome; MYH9 related disorders; Donnai Barrow syndrome; Severe congenital neutropenia and 6, autosomal recessive; Charcot-Marie-Tooth disease, types ID and IVF; Coffin-Lowry syndrome; mitochondrial 3-hydroxy-3-methylglutaryl-CoA synthase deficiency; Hypomagnesemia, seizures, and mental retardation; Ischiopatellar dysplasia; Multiple congenital anomalies-hypotonia-seizures syndrome 3; Spastic paraplegia 50, autosomal recessive; Short stature with nonspecific skeletal abnormalities; Severe myoclonic epilepsy in infancy, Propionic academia; Adolescent nephronophthisis; Macrocephaly, macrosomia, facial dysmorphism syndrome; Stargardt disease 4; Ehlers-Danlos syndrome type 7 (autosomal recessive), classic type, type 2 (progeroid), hydroxylysine-deficient, type 4, type 4 variant, and due to tenascin-X deficiency; Myopia 6; Coxa plana; Familial cold autoinflammatory syndrome 2; Malformation of the heart and great vessels; von Willebrand disease type 2M and type 3; Deficiency of galactokinase; Brugada syndrome 1, X-linked ichthyosis with steryl-sulfatase deficiency; Congenital ocular coloboma; Histiocytosis-lymphadenopathy plus syndrome, Aniridia, cerebellar ataxia, and mental retardation: Left ventricular noncompaction 3; Amyotrophic lateral sclerosis types 1, 6, 15 (with or without frontotemporal dementia), 22 (with or without frontotemporal dementia), and 10; Osteogenesis imperfecta type 12, type 5, type 7, type 8, type I, type III, with normal sclerae, dominant form, recessive perinatal lethal; Hematologic neoplasm; Favism, susceptibility to; Pulmonary Fibrosis And/Or Bone Marrow Failure, Telomere-Related. 1 and 3; Dominant hereditary optic atrophy; Dominant dystrophic epidermolysis bullosa with absence of skin; Muscular dystrophy, congenital, megaconial type; Multiple gastrointestinal atresias; McCune-Albright syndrome; Nail-patella syndrome; McLeod neuroacanthocytosis syndrome; Common variable immunodeficiency 9; Partial hypoxanthine-guanine phosphoribosyltransferase deficiency; Pseudohypoaldosteronism type I autosomal dominant and recessive and type 2; Urocanate hydratase deficiency, Heterotopia; Meckel syndrome type 7, Ch\xc3\xa9diak-Higashi syndrome, Chediak-Higashi syndrome, adult type: Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Insulin resistance, Deficiency of steroid 11-beta-monooxygenase; Popliteal pterygium syndrome; Pulmonary arterial hypertension related to hereditary hemorrhagic telangiectasia; Deafness, autosomal recessive IA, 2, 3, 6, 8, 9, 12, 15, 16, 18b, 22, 28, 31, 44, 49, 63, 77, 86, and 89; Primary hyperoxaluria, type 1, type, and type 11I; Paramyotonia congenita of von Eulenburg; Desbuquois syndrome; Camitine palmitoyltransferase I, II, II (late onset), and II (infantile) deficiency; Secondary hypothyroidism; Mandibulofacial dysostosis, Treacher Collins type, autosomal recessive; Cowden syndrome 1; Li-Fraumeni syndrome 1; Asparagine synthetase deficiency, Malattia leventinese; Optic atrophy 9; Infantile convulsions and paroxysmal choreoathetosis, familial; Ataxia with vitamin E deficiency; Islet cell hyperplasia; Miyoshi muscular dystrophy 1; Thrombophilia, hereditary, due to protein C deficiency, autosomal dominant and recessive; Fechtner syndrome; Properdin deficiency, X-linked; Mental retardation, stereotypic movements, epilepsy, and/or cerebral malformations; Creatine deficiency, X-linked, Pilomatrixoma; Cyanosis, transient neonatal and atypical nephropathic; Adult onset ataxia with oculomotor apraxia; Hemangioma, capillary infantile; PC-K6a; Generalized dominant dystrophic epidermolysis bullosa; Pelizaeus-Merzbacher disease; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Benign familial neonatal seizures I and 2; Primary pulmonary hypertension: Lymphedema, primary, with myelodysplasia; Congenital long QT syndrome; Familial exudative vitreoretinopathy, X-linked; Autosomal dominant hypohidrotic ectodermal dysplasia; Primordial dwarfism; Familial pulmonary capillary hemangiomatosis, Carnitine acylcamitine translocase deficiency: Visceral myopathy; Familial Mediterranean fever and Familial mediterranean fever, autosomal dominant, Combined partial and complete 17-alpha-hydroxylase/17, 20-lyase deficiency; Oto-palato-digital syndrome, type I; Nephrolithiasis/osteoporosis, hypophosphatemic, 2; Familial type 1 and 3 hyperlipoproteinemia; Phenotypes: CHARGE association; Fuhrmann syndrome; Hypotrichosis-lymphedema-telangiectasia syndrome; Chondrodysplasia Blomstrand type, Acroerythrokeratoderma; Slowed nerve conduction velocity, autosomal dominant; Hereditary cancer-predisposing syndrome; Craniodiaphyseal dysplasia, autosomal dominant; Spinocerebellar ataxia autosomal recessive I and 16; Proprotein convertase 1/3 deficiency, D-2-hydroxyglutaric aciduria 2; Hyperekplexia 2 and Hyperekplexia hereditary; Central core disease: Opitz G/BBB syndrome; Cystic fibrosis; Thiel-Behnke corneal dystrophy, Deficiency of bisphosphoglycerate mutase; Mitochondrial short-chain Enoyl-CoA Hydratase 1 deficiency; Ectodermal dysplasia skin fragility syndrome; Wolfram-like syndrome, autosomal dominant; Microcytic anemia; Pyruvate carboxylase deficiency; Leukocyte adhesion deficiency type I and III; Multiple endocrine neoplasia, types land 4; Transient bullous dermolysis of the newborn: Primrose syndrome; Non-small cell lung cancer; Congenital muscular dystrophy; Lipase deficiency combined; COLE-CARPENTER SYNDROME 2; Atrioventricular septal defect and common atrioventricular junction; Deficiency of xanthine oxidase, Waardenburg syndrome type 1, 4C, and 2E (with neurologic involvement); Stickler syndrome, types l(nonsyndromic ocular) and 4; Comeal fragility keratoglobus, blue sclerae and joint hypermobility; Microspherophakia; Chudley-McCullough syndrome; Epidermolysa bullosa simplex and limb girdle muscular dystrophy, simplex with mottled pigmentation, simplex with pyloric atresia, simplex, autosomal recessive, and with pyloric atresia; Rett disorder; Abnormality of neuronal migration; Growth hormone deficiency with pituitary anomalies; Leigh disease; Keratosis palmoplantaris striata 1; Weissenbacher-Zweynuller syndrome; Medium-chain acyl-coenzyme A dehydrogenase deficiency; UDPglucose-4-epimerase deficiency: susceptibility to Autism, X-linked 3; Rhegmatogenous retinal detachment, autosomal dominant; Familial febrile seizures 8; Ulna and fibula absence of with severe limb deficiency; Left ventricular noncompaction 6; Centromeric instability of chromosomes 1.9 and 16 and immunodeficiency; Hereditary diffuse leukoencephalopathy with spheroids; Cushing syndrome: Dopamine receptor d2, reduced brain density of: C-like syndrome; Renal dysplasia, retinal pigmentary dystrophy, cerebellar ataxia and skeletal dysplasia; Ovarian dysgenesis 1; Pierson syndrome; Polyneuropathy, hearing loss, ataxia, retinitis pigmentosa, and cataract, Progressive intrahepatic cholestasis; autosomal dominant, autosomal recessive, and X-linked recessive Alport syndromes; Angelman syndrome; Amish infantile epilepsy syndrome: Autoimmune lymphoproliferative syndrome, type la; Hydrocephalus: Marfanoid habitus; Bare lymphocyte syndrome type 2, complementation group E: Recessive dystrophic epidermolysis bullosa; Factor 1-I, V11, X, v and factor viii, combined deficiency of 2, xiii, a subunit, deficiency; Zonular pulverulent cataract 3; Warts, hypogammaglobulinemia, infections, and myelokathexis; Benign hereditary chorea: Deficiency of hyaluronoglucosaminidase; Microcephaly, hiatal hernia and nephrotic syndrome; Growth and mental retardation, mandibulofacial dysostosis, microcephaly, and cleft palate; Lymphedema, hereditary, id; Delayed puberty; Apparent mineralocorticoid excess, Generalized arterial calcification of infancy 2; METHYLMALONIC ACIDURIA, mut(0) TYPE; Congenital heart disease, multiple types, 2, Familial hypoplastic, glomerulocystic kidney; Cerebrooculofacioskeletal syndrome 2; Stargardt disease 1; Mental retardation, autosomal recessive 15, 44, 46, and 5; Prolidase deficiency; Methylmalonic aciduria cblB type, Oguchi disease; Endocrine-cerebroosteodysplasia; Lissencephaly 1, 2 (X-linked), 3, 6 (with microcephaly), X-linked: Somatotroph adenoma; Gamstorp-Wohlfart syndrome; Lipid proteinosis; Inclusion body myopathy 2 and 3; Enlarged vestibular aqueduct syndrome; Osteoporosis with pseudoglioma: Acquired long QT syandrome; Phenylketonuria; CHOPS syndrome, Global developmental delay; Bietti crystalline corneoretinal dystrophy; Noonan syndrome-like disorder with or without juvenile myelomonocytic leukemia; Congenital erythropoietic porphyria, Atrophia bulborum hereditaria; Paragangliomas 3; Van der Woude syndrome; Aromatase deficiency; Birk Barel mental retardation dysmorphism syndrome; Amyotrophic lateral sclerosis type 5; Methemoglobinemia types 11 and 2; Congenital stationary night blindness, type 1A, B, 1C, IE, IF, and 2A; Seizures, Thyroid cancer, follicular; Lethal congenital contracture syndrome 6; Distal hereditary motor neuronopathy type 2B: Sex cord-stromal tumor; Epileptic encephalopathy, childhood-onset, early infantile, 1, 19, 23, 25, 30, and 32; Myofibrillar myopathy 1 and ZASP-related; Cerebellar ataxia infantile with progressive external ophthalmoplegia; Purine-nucleoside phosphorylase deficiency; Forebrain defects; Epileptic encephalopathy Lennox-Gastaut type: Obesity; 4, Left ventricular noncompaction 10; Verheij syndrome, Mowat-Wilson syndrome; Odontotrichomelic syndrome, Patterned dystrophy of retinal pigment epithelium; Lig4 syndrome; Barakat syndrome; IRAK4 deficiency; Somatotroph adenoma; Branched-chain ketoacid dehydrogenase kinase deficiency; Cystinuria, Familial aplasia of the vermis; Succinyl-CoA acetoacetate transferase deficiency; Scapuloperoneal spinal muscular atrophy; Pigmentary retinal dystrophy; Glanzmann thrombasthenia; Primary open angle glaucoma juvenile onset 1; Aicardi Goutieres syndromes 1, 4, and 5; Renal dysplasia; Intrauterine growth retardation, metaphyseal dysplasia, adrenal hypoplasia congenita, and genital anomalies, Beaded hair; Short stature, onychodysplasia, facial dysmorphism, and hypotrichosis; Metachromatic leukodystrophy; Cholestanol storage disease; Three M syndrome 2; Leber congenital anaurosis 11, 12, 13, 16, 4, 7, and 9; Mandibuloacral dysplasia with type A or B lipodystrophy, atypical; Meier-Gorlin syndromes land 4; Hypotrichosis 8 and 12; Short QT syndrome 3; Ectodermal dysplasia 1 ib, Anonychia; Pseudohypoparathyroidism type 1A, Pseudopseudohvpoparathvroidism; Leber optic atrophy; Bainbridge-Ropers syndrome; Weaver syndrome; Short stature, auditory canal atresia, mandibular hypoplasia, skeletal abnormalities: Deficiency of alpha-mannosidase; Macular dystrophy, vitelliform, adult-onset; Glutaric aciduria, type 1; Gangliosidosis GM1 typel (with cardiac involvenment) 3; Mandibuloacral dysostosis; Hereditary lymphedema type I, Atrial standstill 2; Kabuki make-up syndrome; Bethlem myopathy and Bethlem myopathy 2; Myeloperoxidase deficiency, Fleck corneal dystrophy; Hereditary acrodermatitis enteropathica; Hypobetalipoproteinemia, familial, associated with apob32; Cockayne syndrome type A,; Hyperparathyroidism, neonatal severe; A taxia-telangiectasia-like disorder: Pendred syndrome; I blood group system: Familial benign pemphigus; Visceral heterotaxy 5, autosomal; Nephrogenic diabetes insipidus, Nephrogenic diabetes insipidus, X-linked; Minicore myopathy with external ophthalmoplegia; Perry syndrome; hypohidrotic/hair/tooth type, autosomal recessive: Hereditary pancreatitis; Mental retardation and microcephaly with pontine and cerebellar hypoplasia; Glycogen storage disease 0 (muscle), II (adult form), IXa2, IXc, type IA, Osteopathia striata with cranial sclerosis; Gluthathione synthetase deficiency; Brugada syndrome and Brugada syndrome 4; Endometrial carcinoma, Hypohidrotic ectodermal dysplasia with immune deficiency; Cholestasis, intrahepatic, of pregnancy 3; Bemard-Soulier syndrome, types A1 and A2 (autosomal dominant); Salla disease; Ornithine aminotransferase deficiency; PTEN hamartoma tumor syndrome; Distichiasis-lymphedema syndrome; Corticosteroid-binding globulin deficiency; Adult neuronal ceroid lipofiscinosis; Dejerine-Sottas disease; Tetraamelia, autosomal recessive; Senior-Loken syndrome 4 and 5, Glutaric acidemia IIA and IIB; Aortic aneurysm, familial thoracic 4, 6, and 9; Hyperphosphatasia with mental retardation syndrome 2, 3, and 4; Dyskeratosis congenita X-linked; Arthrogryposis, renal dysfunction, and cholestasis 2; Bannayan-Riley-Ruvalcaba syndrome; 3-Methylglutaconic aciduria; Isolated 17,20-lyase deficiency; Gorlin syndrome; Hand foot uterus syndrome; Tay-Sachs disease, B1 variant, Gm2-gangliosidosis (adult), Gm2-gangliosidosis (adult-onset); Dowling-degos disease 4; Parkinson disease 14, 15, 19 (juvenile-onset), 2, 20 (early-onset), 6, (autosomal recessive early-onset, and 9; Ataxia, sensory, autosomal dominant; Congenital microvillous atrophy; Myoclonic-Atonic Epilepsy; Tangier disease; 2-methyl-3-hydroxybutyric aciduria; Familial renal hypouricemia; Schizencephaly; Mitochondrial DNA depletion syndrome 4B, MNGIE type; Feingold syndrome 1; Renal carnitine transport defect; Familial hypercholesterolemia, Townes-Brocks-branchiootorenal-like syndrome; Griscelli syndrome type 3; Meckel-Gruber syndrome; Bullous ichthyosiform erythroderma; Neutrophil immunodeficiency syndrome; Myasthenic Syndrome, Congenital, 17, 2A (slow-channel), 4B (fast-channel), and without tubular aggregates; Microvascular complications of diabetes 7; McKusick Kaufman syndrome; Chronic granulomatous disease, autosomal recessive cytochrome b-positive, types 1 and 2; Arginino succinate lyase deficiency; Mitochondrial phosphate carrier and pyruvate carrier deficiency; Lattice corneal dystrophy Type III, Ectodermal dysplasia-syndactyly syndrome 1, Hypomyelinating leukodystrophy 7; Mental retardation, autosomal dominant 12, 13, 15, 24, 3, 30, 4, 5, 6, and 9; Generalized epilepsy with febrile seizures plus, types 1 and 2; Psoriasis susceptibility 2; Frank Ter Haar syndrome; Thoracic aortic aneurysms and aortic dissections; Crouzon syndrome; Granulosa cell tumor of the ovary; Epidermolytic palmoplantar keratoderma, Leri Weill dyschondrosteosis; 3 beta-Hydroxysteroid dehydrogenase deficiency; Familial restrictive cardiomyopathy 1; Autosomal dominant progressive external ophthalmoplegia with mitochondrial DNA deletions 1 and 3; Antley-Bixler syndrome with genital anomalies and disordered steroidogenesis; Hajdu-Cheney syndrome; Pigmented nodular adrenocortical disease, primary, 1, Episodic pain syndrome, familial, 3; Dejerine-Sottas syndrome, autosomal dominant, FG syndrome and FG syndrome 4; Dendritic cell, monocyte, B lymphocyte, and natural killer lymphocyte deficiency; Hypothyroidism, congenital, nongoitrous, 1; Miller syndrome; Nemaline myopathy 3 and 9; Oligodontia-colorectal cancer syndrome; Cold-induced sweating syndrome 1, Van Buchem disease type 2; Glaucoma 3, primary congenital, d; Citrullinemia type I and II; Nonaka myopathy; Congenital muscular dystrophy due to partial LAMA2 deficiency; Myoneural gastrointestinal encephalopathy syndrome; Leigh syndrome due to mitochondrial complex I deficiency; Medulloblastoma; Pyruvate dehydrogenase El-alpha deficiency; Carcinoma of colon; Nance-Horan syndrome, Sandhoff disease, adult and infantil types; Arthrogryposis renal dysfunction cholestasis syndrome; Autosomal recessive hypophosphatemic bone disease; Doyne honeycomb retinal dystrophy; Spinocerebellar ataxia 14, 21, 35, 40, and 6; Lewy body dementia; RRM2B-related mitochondrial disease; Brody myopathy, Megalencephaly-polymicrogyria-polydactyly-hydrocephalus syndrome 2; Usher syndrome, types 1, IB, ID, 1G, 2A, 2C, and 2D; hypocalcification type and hypomaturation type, IIA1 Amelogenesis imperfecta, Pituitary hormone deficiency, combined 1, 2, 3, and 4; Cushing symphalangism; Renal tubular acidosis, distal, autosomal recessive, with late-onset sensorineural hearing loss, or with hemolytic anemia, Infantile nephronophthisis; Juvenile polyposis syndrome; Sensory ataxic neuropathy, dysarthria, and ophthalmoparesis; Deficiency of 3-hydroxyacyl-CoA dehydrogenase, Parathyroid carcinoma; X-linked agammaglobulinemia; Megaloblastic anemia, thiamine-responsive, with diabetes mellitus and sensorineural deafness; Multiple sulfatase deficiency; Neurodegeneration with brain iron accumulation 4 and 6; Cholesterol monooxygenase (side-chain cleaving) deficiency; hemolytic anemia due to Adenylosuccinate lyase deficiency; Myoclonus with epilepsy with ragged red fibers; Pitt-Hopkins syndrome; Multiple pterygium syndrome Escobar type; Homocystinuria-Megaloblastic anemia due to defect in cobalamin metabolism. cblE complementation type; Cholecystitis; Spherocytosis types 4 and 5; Multiple congenital anomalies; Xeroderma pigmentosum, complementation group b, group D. group E. and group G; Leiner disease; Groenouw corneal dystrophy type 1; Coenzyme Q10 deficiency, primary 1, 4, and 7; Distal spinal muscular atrophy, congenital nonprogressive; Warburg micro syndrome 2 and 4; Bile acid synthesis defect, congenital, 3; Acth-independent macronodular adrenal hyperplasia 2; Acrocapitofemoral dysplasia; Paget disease of bone, familial; Severe neonatal-onset encephalopathy with microcephaly; Zimmermann-Laband syndrome and Zimmermann-Laband syndrome 2; Reifenstein syndrome; Familial hypokalemia-hypomagnesemia, Photosensitive trichothiodystrophy; Adult junctional epidermolysis bullosa; Lung cancer; Freeman-Sheldon syndrome; Hyperinsulinism-hyperammonemia syndrome; Posterior polar cataract type 2: Sclerocornea, autosomal recessive; Juvenile GM.%>1<gangliosidosis; Cohen syndrome, Hereditary Paraganglioma-Pheochromocytoma Syndromes, Neonatal insulin-dependent diabetes mellitus; Hypochondrogenesis; Floating-Harbor syndrome; Cutis laxa with osteodystrophy and with severe pulmonary, gastrointestinal, and urinary abnormalities; Congenital contractures of the limbs and face, hypotonia, and developmental delay: Dyskeratosis congenita autosomal dominant and autosomal dominant, 3; Histiocytic medullary reticulosis; Costello syndrome, Immunodeficiency 15, 16, 19, 30, 31C, 38, 40, 8, due to defect in cd3-zeta, with hyper IgM type 1 and 2, and X-Linked, with magnesium defect, Epstein-Barr vims infection, and neoplasia; Atrial septal defects 2, 4, and 7 (with or without atrioventricular conduction defects); GTP cyclohydrolase I deficiency; Talipes equinovarus; Phosphoglycerate kinase I deficiency: Tuberous sclerosis 1 and 2; Autosomal recessive congenital ichthyosis 1, 2, 3, 4A, and 4B; and Familial hypertrophic cardiomyopathy 1, 2, 3, 4, 7, 10, 23 and 24.

### 1.1.3.2 Indications by Tissue

Additional suitable diseases and disorders that can be treated by the systems and methods provided herein include, without limitation, diseases of the central nervous system (CNS) (see exemplary diseases and affected genes in Table 13), diseases of the eye (see exemplary diseases and affected genes in Table 14), diseases of the heart (see exemplary diseases and affected genes in Table 15), diseases of the hematopoietic stem cells (HSC) (see exemplary diseases and affected genes in Table 16), diseases of the kidney (see exemplary diseases and affected genes in Table 17), diseases of the liver (see exemplary diseases and affected genes in Table 18), diseases of the lung (see exemplary diseases and affected genes in Table 19), diseases of the skeletal muscle (see exemplary diseases and affected genes in Table 20), and diseases of the skin (see exemplary diseases and affected genes in Table 21). Table 22 provides exemplary protective mutations that reduce risks of the indicated diseases. In some embodiments, a Gene Writer system described herein is used to treat an indication of any of Tables 13-21. In some embodiments, the GeneWriter system modifies a target site in genomic DNA in a cell, wherein the target site is in a gene of any of Tables 13-21, e.g., in a subject having the corresponding indication listed in any of Tables 13-21. In some embodiments, the GeneWriter corrects a mutation in the gene. In some embodiments, the GeneWriter inserts a sequence that had been deleted from the gene (e.g., through a disease-causing mutation). In some embodiments, the GeneWriter deletes a sequence that had been duplicated in the gene (e.g., through a disease-causing mutation). In some embodiments, the GeneWriter replaces a mutation (e.g., a disease-causing mutation) with the corresponding wild-type sequence. In some embodiments, the mutation is a substitution, insertion, deletion, or inversion.

TABLE 13
1.1.3.2.1 CNS diseases and genes affected.

| Disease | Gene Affected |
|---|---|
| Alpha-mannosidosis | MAN2B1 |
| Ataxia-telangiectasia | ATM |
| CADASIL | NOTCH3 |
| Canavan disease | ASPA |
| Carbamoyl-phosphate synthetase 1 deficiency | CPS1 |
| CLN1 disease | PPT1 |
| CLN2 Disease | TPP1 |
| CLN3 Disease (Juvenile neuronal ceroid | CLN3 |
| lipofuscinosis, Batten Disease) | |
| Coffin-Lowry syndrome | RPS6KA3 |
| Congenital myasthenic syndrome 5 | COLQ |
| Cornelia de Lange syndrome (NIPBL) | NIPBL |
| Cornelia de Lange syndrome (SMC1A) | SMC1A |
| Dravet syndrome (SCN1A) | SCN1A |
| Glycine encephalopathy (GLDC) | GLDC |
| GM1 gangliosidosis | GLB1 |
| Huntington's Disease | HTT |
| Hydrocephalus with stenosis of the aqueduct | L1CAM |
| of Sylvius | |
| Leigh Syndrome | SURF1 |
| Metachromatic leukodystrophy (ARSA) | ARSA |
| MPS type 2 | IDS |
| MPS type 3 | Type 3a: SGSH |
| | Type 3b: NAGLU |
| Mucolipidosis IV | MCOLN1 |
| Neurofibromatosis Type 1 | NF1 |
| Neurofibromatosis type 2 | NF2 |
| Pantothenate kinase-associated neurodegeneration | PANK2 |
| Pyridoxine-dependent epilepsy | ALDH7A1 |
| Rett syndrome (MECP2) | MECP2 |
| Sandhoff disease | HEXB |
| Semantic dementia (Frontotemporal dementia) | MAPT |
| Spinocerebellar ataxia with axonal neuropathy | SETX |
| (Ataxia with Oculomotor Apraxia) | |
| Tay-Sachs disease | HEXA |
| X-linked Adrenoleukodystrophy | ABCD1 |

TABLE 14
1.1.3.2.2 Eye diseases and genes affected.

| Disease | Gene Affected |
|---|---|
| Achromatopsia | CNGB3 |
| Amaurosis Congenita (LCA1) | GUCY2D |
| Amaurosis Congenita (LCA10) | CEP290 |
| Amaurosis Congenita (LCA2) | RPE65 |
| Amaurosis Congenita (LCA8) | CRB1 |
| Choroideremia | CHM |
| Cone Rod Dystrophy (ABCA4) | ABCA4 |
| Cone Rod Dystrophy (CRX) | CRX |
| Cone Rod Dystrophy (GUCY2D) | GUCY2D |
| Cystinosis, Ocular Nonnephropathic | CTNS |
| Lattice corneal dystrophy type I | TGFBI |
| Macular Corneal Dystrophy (MCD) | CHST6 |
| Optic Atrophy | OPA1 |
| Retinitis Pigmentosa (AR) | USH2A |
| Retinitis Rigmentosa (AD) | RHO |
| Stargardt Disease | ABCA4 |
| Vitelliform Macular Dystrophy | BEST1; PRPH2 |

TABLE 15
1.1.3.2.3 Heart diseases and genes affected.

| | Gene |
|---|---|
| Disease | Affected |
| Arrhythmogenic right ventricular cardiomyopathy (ARVC) | PKP2 |
| Barth syndrome | TAZ |
| Becker muscular dystrophy | DMD |
| Brugada syndrome | SCN5A |
| Catecholaminergic polymorphic ventricular tachycardia | RYR2 |
| (RYR2) | |
| Dilated cardiomyopathy (LMNA) | LMNA |
| Dilated cardiomyopathy (TTN) | TTN |
| Duchenne muscular dystrophy | DMD |
| Emery-Dreifuss Muscular Dystrophy Type I | EMD |
| Familial hypertrophic cardiomyopathy | MYH7 |
| Familial hypertrophic cardiomyopathy | MYBPC3 |
| Jervell Lange-Nielsen syndrome | KCNQ1 |
| LCHAD deficiency | HADHA |
| Limb-girdle muscular dystrophy type 1B (Emery-Dreifuss | LMNA |
| EDMD2) | |
| Limb-girdle muscular dystrophy, type 2D | SGCA |
| Long QT syndrome 1 (Romano Ward) | KCNQ1 |

TABLE 16
1.1.3.2.4 HSC diseases and genes affected.

| Disease | Gene Affected |
|---|---|
| ADA-SCID | ADA |
| Adrenoleukodystrophy (CALD) | ABCD1 |
| Alpha-mannosidosis | MAN2B1 |
| Chronic granulomatous disease | CYBB; CYBA; |
| | NCF1; NCF2; NCF4 |
| Common variable immunodeficiency | TNFRSF13B |
| Fanconi anemia | FANCA; FANCC; |
| | FANCG |
| Gaucher disease | GBA |
| Globoid cell leukodystrophy (Krabbe disease) | GALC |
| Hemophagocytic lymphohistiocytosis | PRF1; STX11; |
| | STXBP2; UNC13D |
| IL-7R SCID | IL7R |
| JAK-3 SCID | JAK3 |
| Malignant infantile osteopetrosis- autosomal | TCIRG1; Many genes |
| recessive osteopetrosis | implicated |
| Metachromatic leukodystrophy | ARSA; PSAP |
| MPS 1S (Scheie syndrome) | IDUA |
| MPS2 | IDS |
| MPS7 | GUSB |
| Mucolipidosis II | GNPTAB |
| Niemann-Pick disease A and B | SMPD1 |
| Niemann-Pick disease C | NPC1 |
| Paroxysmal Nocturnal Hemoglobinuria | PIGA |
| Pompe disease | GAA |
| Pyruvate kinase deficiency (PKD) | PKLR |
| RAG ½ Deficiency (SCID with granulomas) | RAG1/RAG2 |
| Severe Congenital Neutropenia | ELANE; HAX1 |
| Sickle cell disease (SCD) | HBB |
| Tay Sachs | HEXA |
| Thalassemia | HBB |
| Wiskott-Aldrich Syndrome | WAS |
| X-linked agammaglobulinemia | BTK |
| X-linked SCID | IL2RG |

TABLE 17
1.1.3.2.5 Kidney diseases and genes affected.

| | Gene |
|---|---|
| Disease | Affected |
| Alport syndrome | COL4A5 |
| Autosomal dominant polycystic kidney disease (PKD1) | PKD1 |
| Autosomal dominant polycystic kidney disease (PKD2) | PDK2 |
| Autosomal dominant tubulointerstitial kidney disease (MUC1) | MUC1 |
| Autosomal dominant tubulointerstitial kidney disease | UMOD |
| (UMOD) | |
| Autosomal recessive polycystic kidney disease | PKHD1 |
| Congenital nephrotic syndrome | NPHS2 |
| Cystinosis | CTNS |

TABLE 18
1.1.3.2.6 Liver diseases and genes affected.

| Disease | Gene Affected |
|---|---|
| Acute intermittent porphyria | HMBS |
| Alagille syndrome | JAG1 |
| Alpha-1-antitrypsin deficiency | SERPINA1 |
| Carbamoyl phosphate synthetase I | CPS1 |
| deficiency | |
| Citrullinemia I | ASS1 |
| Crigler-Najjar | UGT1A1 |
| Fabry | LPL |
| Familial chylomicronemia syndrome | GLA |
| Gaucher | GBE1 |
| GSD IV | GBA |
| Heme A | F8 |
| Heme B | F9 |
| Hereditary amyloidosis (hTTR) | TTR |
| Hereditary angioedema | SERPING1 |
| | (KLKB1 for CRISPR) |
| HoFH | LDLRAP1 |
| Hypercholesterolemia | PCSK9 |
| Methylmalonic acidemia | MMUT |
| MPS II | IDS |
| MPS III | Type IIIa: SGSH |
| | Type IIIb: NAGLU |
| | Type IIIc: HGSNAT |
| | Type IIId: GNS |
| MPS IV | Type IVA: GALNS |
| | Type IVB: GLB1 |
| MPS VI | ARSB |
| MSUD | Type Ia: BCKDHA |
| | Type Ib: BCKDHB |
| | Type II: DBT |
| OTC Deficiency | OTC |
| Polycystic Liver Disease | PRKCSH |
| Pompe | GAA |
| Primary Hyperoxaluria 1 | AGXT (HAO1 or |
| | LDHA for CRISPR) |
| Progressive familial intrahepatic | ATP8B1 |
| cholestasis type 1 | |
| Progressive familial intrahepatic | ABCB11 |
| cholestasis type 2 | |
| Progressive familial intrahepatic | ABCB4 |
| cholestasis type 3 | |
| Propionic acidemia | PCCB; PCCA |
| Wilson's Disease | ATP7B |
| Glycogen storage disease, Type 1a | G6PC |
| Glycogen storage disease, Type IIIb | AGL |
| Isovaleric acidemia | IVD |
| Wolman disease | LIPA |

TABLE 19
1.1.3.2.7 Lung diseases and genes affected.

| Disease | Gene Affected |
|---|---|
| Alpha-1 antitrypsin deficiency | SERPINA1 |
| Cystic fibrosis | CFTR |
| Primary ciliary dyskinesia | DNAI1 |
| Primary ciliary dyskinesia | DNAH5 |
| Primary pulmonary hypertension I | BMPR2 |
| Surfactant Protein B (SP-B) Deficiency (pulmonary | SFTPB |
| surfactant metabolism dysfunction 1) | |

TABLE 20
1.1.3.2.8 Skeletal muscle diseases and genes affected.

| Disease | Gene Affected |
|---|---|
| Becker muscular dystrophy | DMD |
| Becker myotonia | CLCN1 |
| Bethlem myopathy | COL6A2 |
| Centronuclear myopathy, X-linked (myotubular) | MTM1 |
| Congenital myasthenic syndrome | CHRNE |
| Duchenne muscular dystrophy | DMD |
| Emery-Dreifuss muscular dystrophy, AD | LMNA |
| Facioscapulohumeral Muscular Dystrophy | DUX4 - D4Z4 |
| | chromosomal region |
| Hyperkalemic periodic paralysis | SCN4A |
| Hypokalemic periodic paralysis | CACNA1S |
| Limb-girdle muscular dystrophy 2A | CAPN3 |
| Limb-girdle muscular dystrophy 2B | DYSF |
| Limb-girdle muscular dystrophy, type 2D | SGCA |
| Miyoshi muscular dystrophy 1 | DYSF |
| Paramyotonia congenita | SCN4A |
| Thomsen myotonia | CLCN1 |
| VCP myopathy (IBMPFD) 1 | VCP |

TABLE 21
1.1.3.2.9 Skin diseases and genes affected.

| Disease | Gene Affected |
|---|---|
| Epidermolysis Bullosa Dystrophica Dominant | COL7A1 |
| Epidermolysis Bullosa Dystrophica Recessive | COL7A1 |
| (Hallopeau-Siemens) | |
| Epidermolysis Bullosa Junctional | LAMB3 |
| Epidermolysis Bullosa Simplex | KRT5; KRT14 |
| Epidermolytic Ichthyosis | KRT1; KRT10 |
| Hailey-Hailey Disease | ATP2C1 |
| Lamellar Ichthyosis/Nonbullous Congenital | TGM1 |
| Ichthyosiform Erythroderma (ARCI) | |
| Netherton Syndrome | SPINK5 |

### 1.1.3.3 Regulatory Edits

In some embodiments, the systems or methods provided herein can be used to introduce a regulatory edit. In some embodiments, the regulatory edit is introduced to a regulatory sequence of a gene, for example, a gene promoter, gene enhancer, gene repressor, or a sequence that regulates gene splicing. In some embodiments, the regulatory edit increases or decreases the expression level of a target gene. In some embodiments, the target gene is the same as the gene containing a disease-causing mutation. In some embodiment, the target gene is different from the gene containing a disease-causing mutation. For example, the systems or methods provided herein can be used to upregulate the expression of fetal hemoglobin by introducing a regulatory edit at the promoter of bcl11a, thereby treating sickle cell disease.

### 1.1.3.4 Exemplary Heterologous Object Sequences

In some embodiments, the systems or methods provided herein comprise a heterologous object sequence, wherein the heterologous object sequence or a reverse complementary sequence thereof, encodes a protein (e.g., an antibody) or peptide. In some embodiments, the therapy is one approved by a regulatory agency such as FDA.

In some embodiments, the protein or peptide is a protein or peptide from the THPdb database (Usmani et al. PLoS One 12(7):e0181748 (2017), herein incorporated by reference in its entirety. In some embodiments, the protein or peptide is a protein or peptide disclosed in Table 28. In some embodiments, the systems or methods disclosed herein, for example, those comprising Gene Writers, may be used to integrate an expression cassette for a protein or peptide from Table 28 into a host cell to enable the expression of the protein or peptide in the host. In some embodiments, the sequences of the protein or peptide in the first column of Table 28 can be found in the patents or applications provided in the third column of Table 28, incorporated by reference in their entireties.

In some embodiments, the protein or peptide is an antibody disclosed in Table 1 of Lu et al. J Biomed Sci 27(1):1 (2020), herein incorporated by reference in its entirety. In some embodiments, the protein or peptide is an antibody disclosed in Table 29. In some embodiments, the systems or methods disclosed herein, for example, those comprising Gene Writers, may be used to integrate an expression cassette for an antibody from Table 29 into a host cell to enable the expression of the antibody in the host. In some embodiments, a system or method described herein is used to express an agent that binds a target of column 2 of Table 29 (e.g., a monoclonal antibody of column 1 of Table 29) in a subject having an indication of column 3 of Table 29.

TABLE 28
1.1.3.4.1 Exemplary protein and peptide therapeutics.

| Therapeutic peptide | Category | Patent Number |
|---|---|---|
| Lepirudin | Antithrombins and Fibrinolytic Agents | CA1339104 |
| Cetuximab | Antineoplastic Agents | CA1340417 |
| Dor se alpha | Enzymes | CA2184581 |
| Denileukin diftitox | Antineoplastic Agents | |
| Etanercept | Immunosuppressive Agents | CA2476934 |
| Bivalirudin | Antithrombins | U.S. Pat. No. 7,582,727 |
| Leuprolide | Antineoplastic Agents | |
| Peginterferon alpha-2a | Immunosuppressive Agents | CA2203480 |
| Alteplase | Thrombolytic Agents | |
| Interferon alpha-n1 | Antiviral Agents | |
| Darbepoetin alpha | Anti-anemic Agents | CA2165694 |
| Reteplase | Fibrinolytic Agents | CA2107476 |
| Epoetin alpha | Hematinics | CA1339047 |
| Salmon Calcitonin | Bone Density Conservation Agents | U.S. Pat. No. 6,440,392 |
| Interferon alpha-n3 | Immunosuppressive Agents | |
| Pegfilgrastim | Immunosuppressive Agents | CA1341537 |
| Sargramostim | Immunosuppressive Agents | CA1341150 |
| Secretin | Diagnostic Agents | |
| Peginterferon alpha-2b | Immunosuppressive Agents | CA1341567 |
| Asparaginase | Antineoplastic Agents | |
| Thyrotropin alpha | Diagnostic Agents | U.S. Pat. No. 5,840,566 |
| Antihemophilic Factor | Coagulants and Thrombotic agents | CA2124690 |
| A kinra | Antirheumatic Agents | CA2141953 |
| Gramicidin D | Anti-Bacterial Agents | |
| Intravenous Immunoglobulin | Immunologic Factors | |
| Anistreplase | Fibrinolytic Agents | |
| Insulin Regular | Antidiabetic Agents | |
| Tenecteplase | Fibrinolytic Agents | CA2129660 |
| Menotropins | Fertility Agents | |
| Interferon gamma-1b | Immunosuppressive Agents | U.S. Pat. No. 6,936,695 |
| Interferon alpha-2a, Recombinant | | CA2172664 |
| Coagulation factor VIIa | Coagulants | |
| Oprelvekin | Antineoplastic Agents | |
| Palifermin | Anti-Mucositis Agents | |
| Glucagon recombinant | Hypoglycemic Agents | |
| Aldesleukin | Antineoplastic Agents | |
| Botulinum Toxin Type B | Antidystonic Agents | |
| Omalizumab | Anti-Allergic Agents | CA2113813 |
| Lutropin alpha | Fertility Agents | U.S. Pat. No. 5,767,251 |
| Insulin Lispro | Hypoglycemic Agents | U.S. Pat. No. 5,474,978 |
| Insulin Glargine | Hypoglycemic Agents | U.S. Pat. No. 7,476,652 |
| Collage se | | |
| Rasburicase | Gout Suppressants | CA2175971 |
| Adalimumab | Antirheumatic Agents | CA2243459 |
| Imiglucerase | Enzyme Replacement Agents | U.S. Pat. No. 5,549,892 |
| Abciximab | Anticoagulants | CA1341357 |
| Alpha-1-protei se inhibitor | Serine Proteinase Inhibitors | |
| Pegaspargase | Antineoplastic Agents | |
| Interferon beta-1a | Antineoplastic Agents | CA1341604 |
| Pegademase bovine | Enzyme Replacement Agents | |
| Human Serum Albumin | Serum substitutes | U.S. Pat. No. 6,723,303 |
| Eptifibatide | Platelet Aggregation Inhibitors | U.S. Pat. No. 6,706,681 |
| Serum albumin iodo ted | Diagnostic Agents | |
| Infliximab | Antirheumatic Agents, Anti- | CA2106299 |
| | Inflammatory Agents, Non- | |
| | Steroidal, Dermatologic Agents, | |
| | Gastrointestinal Agents and | |
| | Immunosuppressive Agents | |
| Follitropin beta | Fertility Agents | U.S. Pat. No. 7,741,268 |
| Vasopressin | Antidiuretic Agents | |
| Interferon beta-1b | Adjuvants, Immunologic and | CA1340861 |
| | Immunosuppressive Agents | |
| Interferon alphacon-1 | Antiviral Agents and | CA1341567 |
| | Immunosuppressive Agents | |
| Hyaluronidase | Adjuvants, Anesthesia and | |
| | Permeabilizing Agents | |
| Insulin, porcine | Hypoglycemic Agents | |
| Trastuzumab | Antineoplastic Agents | CA2103059 |
| Rituximab | Antineoplastic Agents, Immunologic | CA2149329 |
| | Factors and Antirheumatic Agents | |
| Basiliximab | Immunosuppressive Agents | CA2038279 |
| Muromo b | Immunologic Factors and | |
| | Immunosuppressive Agents | |
| Digoxin Immune Fab (Ovine) | Antidotes | |
| Ibritumomab | | CA2149329 |
| Daptomycin | | U.S. Pat. No. 6,468,967 |
| Tositumomab | | |
| Pegvisomant | Hormone Replacement Agents | U.S. Pat. No. 5,849,535 |
| Botulinum Toxin Type A | Neuromuscular Blocking Agents, Anti- | CA2280565 |
| | Wrinkle Agents and Antidystonic Agents | |
| Pancrelipase | Gastrointestinal Agents and Enzyme | |
| | Replacement Agents | |
| Streptoki se | Fibrinolytic Agents and Thrombolytic | |
| | Agents | |
| Alemtuzumab | | CA1339198 |
| Alglucerase | Enzyme Replacement Agents | |
| Capromab | Indicators, Reagents and Diagnostic | |
| | Agents | |
| Laronidase | Enzyme Replacement Agents | |
| Urofollitropin | Fertility Agents | U.S. Pat. No. 5,767,067 |
| Efalizumab | Immunosuppressive Agents | |
| Serum albumin | Serum substitutes | U.S. Pat. No. 6,723,303 |
| Choriogonadotropin alpha | Fertility Agents and Gonadotropins | U.S. Pat. No. 6,706,681 |
| Antithymocyte globulin | Immunologic Factors and | |
| | Immunosuppressive Agents | |
| Filgrastim | Immunosuppressive Agents, | CA1341537 |
| | Antineutropenic Agents and | |
| | Hematopoietic Agents | |
| Coagulation factor ix | Coagulants and Thrombotic Agents | |
| Becaplermin | Angiogenesis Inducing Agents | CA1340846 |
| Agalsidase beta | Enzyme Replacement Agents | CA2265464 |
| Interferon alpha-2b | Immunosuppressive Agents | CA1341567 |
| Oxytocin | Oxytocics, Anti-tocolytic Agents | |
| | and Labor Induction Agents | |
| Enfuvirtide | HIV Fusion Inhibitors | U.S. Pat. No. 6,475,491 |
| Palivizumab | Antiviral Agents | CA2197684 |
| Daclizumab | Immunosuppressive Agents | |
| Bevacizumab | Angiogenesis Inhibitors | CA2286330 |
| Arcitumomab | Diagnostic Agents | U.S. Pat. No. 8,420,081 |
| Arcitumomab | Diagnostic Agents | U.S. Pat. No. 7,790,142 |
| Eculizumab | | CA2189015 |
| Panitumumab | | |
| Ranibizumab | Ophthalmics | CA2286330 |
| Idursulfase | Enzyme Replacement Agents | |
| Alglucosidase alpha | Enzyme Replacement Agents | CA2416492 |
| Exe tide | Hypoglycemic Agents | U.S. Pat. No. 6,872,700 |
| Mecasermin | | U.S. Pat. No. 5,681,814 |
| Pramlintide | | U.S. Pat. No. 5,686,411 |
| Galsulfase | Enzyme Replacement Agents | |
| Abatacept | Antirheumatic Agents and | CA2110518 |
| | Immunosuppressive Agents | |
| Cosyntropin | Hormones and Diagnostic Agents | |
| Corticotropin | | |
| Insulin aspart | Hypoglycemic Agents and | U.S. Pat. No. 5,866,538 |
| | Antidiabetic Agents | |
| Insulin detemir | Antidiabetic Agents | U.S. Pat. No. 5,750,497 |
| Insulin glulisine | Antidiabetic Agents | U.S. Pat. No. 6,960,561 |
| Pegaptanib | Intended for the prevention of | |
| | respiratory distress syndrome | |
| | (RDS) in premature infants at | |
| | high risk for RDS. | |
| Nesiritide | | |
| Thymalphasin | | |
| Defibrotide | Antithrombins | |
| tural alpha interferon | | |
| OR multiferon | | |
| Glatiramer acetate | | |
| Preotact | | |
| Teicoplanin | Anti-Bacterial Agents | |
| Ca kinumab | Anti-Inflammatory Agents and | |
| | Monoclonal antibodies | |
| Ipilimumab | Antineoplastic Agents and | CA2381770 |
| | Monoclonal antibodies | |
| Sulodexide | Antithrombins and Fibrinolytic | |
| | Agents and Hypoglycemic Agents | |
| | and Anticoagulants and | |
| | Hypolipidemic Agents | |
| Tocilizumab | | CA2201781 |
| Teriparatide | Bone Density Conservation Agents | U.S. Pat. No. 6,977,077 |
| Pertuzumab | Monoclonal antibodies | CA2376596 |
| Rilo cept | Immunosuppressive Agents | U.S. Pat. No. 5,844,099 |
| Denosumab | Bone Density Conservation Agents | CA2257247 |
| | and Monoclo 1 antibodies | |
| Liraglutide | | U.S. Pat. No. 6,268,343 |
| Golimumab | Antipsoriatic Agents and Monoclo 1 | |
| | antibodies andTNF inhibitor | |
| Belatacept | Antirheumatic Agents and | |
| | Immunosuppressive Agents | |
| Buserelin | | |
| Velaglucerase alpha | Enzymes | U.S. Pat. No. 7,138,262 |
| Tesamorelin | | U.S. Pat. No. 5,861,379 |
| Brentuximab vedotin | | |
| Taliglucerase alpha | Enzymes | |
| Belimumab | Monoclonal antibodies | |
| Aflibercept | Antineoplastic Agents and Ophthalmics | U.S. Pat. No. 7,306,799 |
| Asparagi se erwinia chrysanthemi | Enzymes | |
| Ocriplasmin | Ophthalmics | |
| Glucarpidase | Enzymes | |
| Teduglutide | | U.S. Pat. No. 5,789,379 |
| Raxibacumab | Anti-Infective Agents and Monoclonal | |
| | antibodies | |
| Certolizumab pegol | TNF inhibitor | CA2380298 |
| Insulin, isophane | Hypoglycemic Agents and Antidiabetic Agents | |
| Epoetin zeta | | |
| Obinutuzumab | Antineoplastic Agents | |
| Fibrinolysin aka plasmin | | U.S. Pat. No. 3,234,106 |
| Follitropin alpha | | |
| Romiplostim | Colony-Stimulating Factors and | |
| | Thrombopoietic Agents | |
| Luci ctant | Pulmonary surfactants | U.S. Pat. No. 5,407,914 |
| talizumab | Immunosuppressive agents | |
| Aliskiren | Renin inhibitor | |
| Ragweed Pollen Extract | | |
| Secukinumab | Inhibitor | US20130202610 |
| Somatotropin Recombi nt | Hormone Replacement Agents | CA1326439 |
| Drotrecogin alpha | Antisepsis | CA2036894 |
| Alefacept | Dermatologic and Immunosupressive agents | |
| OspA lipoprotein | Vaccines | |
| Uroki se | | U.S. Pat. No. 4,258,030 |
| Abarelix | Anti-Testosterone Agents | U.S. Pat. No. 5,968,895 |
| Sermorelin | Hormone Replacement Agents | |
| Aprotinin | | U.S. Pat. No. 5,198,534 |
| Gemtuzumab ozogamicin | Antineoplastic agents and Immunotoxins | U.S. Pat. No. 5,585,089 |
| Satumomab Pendetide | Diagnostic Agents | |
| Albiglutide | Drugs used in diabetes; alimentary tract and | |
| | metabolism; blood glucose lowering drugs, | |
| | excl. insulins. | |
| Alirocumab | | |
| Ancestim | | |
| Antithrombin alpha | | |
| Antithrombin III human | | |
| Asfotase alpha | Enzymes Alimentary Tract and Metabolism | |
| Atezolizumab | | |
| Autologous cultured chondrocytes | | |
| Beractant | | |
| Bli tumomab | Antineoplastic Agents Immunosuppressive | US20120328618 |
| | Agents Monoclonal antibodies Antineoplastic | |
| | and Immunomodulating Agents | |
| C1 Esterase Inhibitor (Human) | | |
| Coagulation Factor XIII A-Subunit | | |
| (Recombi nt) | | |
| Conestat alpha | | |
| Daratumumab | Antineoplastic Agents | |
| Desirudin | | |
| Dulaglutide | Hypoglycemic Agents; Drugs Used in Diabetes; | |
| | AlimentaryTract and Metabolism; Blood Glucose | |
| | Lowering Drugs, Excl.Insulins | |
| Elosulfase alpha | Enzymes; Alimentary Tract and Metabolism | |
| Elotuzumab | | US2014055370 |
| Evolocumab | Lipid Modifying Agents, Plain; Cardiovascular | |
| | System | |
| Fibrinogen Concentrate (Human) | | |
| Filgrastim-sndz | | |
| Gastric intrinsic factor | | |
| Hepatitis B immune globulin | | |
| Human calcitonin | | |
| Human clostridium tetani | | |
| toxoid immune globulin | | |
| Human rabies virus | | |
| immune globulin | | |
| Human Rho(D) immune | | |
| globulin | | |
| Hyaluronidase (Human | | U.S. Pat. No. 7,767,429 |
| Recombi nt) | | |
| Idarucizumab | Anticoagulant | |
| Immune Globulin Human | Immunologic Factors; | |
| | Immunosuppressive Agents; | |
| | Anti-Infective Agents | |
| Vedolizumab | Immunosupressive agent, | US2012151248 |
| | Antineoplastic agent | |
| Ustekinumab | Deramtologic agent, | |
| | Immunosuppressive agent, | |
| | antineoplastic agent | |
| Turoctocog alpha | | |
| Tuberculin Purified Protein | | |
| Derivative | | |
| Simoctocog alpha | Antihaemorrhagics: blood | |
| | coagulation factor VIII | |
| Siltuximab | Antineoplastic and | U.S. Pat. No. 7,612,182 |
| | Immunomodulating Agents, | |
| | Immunosuppressive Agents | |
| Sebelipase alpha | Enzymes | |
| Sacrosidase | Enzymes | |
| Ramucirumab | Antineoplastic and | US2013067098 |
| | Immunomodulating Agents | |
| Prothrombin complex | | |
| concentrate | | |
| Poractant alpha | Pulmonary Surfactants | |
| Pembrolizumab | Antineoplastic and | US2012135408 |
| | Immunomodulating Agents | |
| Peginterferon beta-1a | | |
| Ofatumumab | Antineoplastic and | U.S. Pat. No. 8,337,847 |
| | Immunomodulating Agents | |
| Obiltoxaximab | | |
| Nivolumab | Antineoplastic and | US2013173223 |
| | Immunomodulating Agents | |
| Necitumumab | | |
| Metreleptin | | US20070099836 |
| Methoxy polyethylene | | |
| glycol-epoetin beta | | |
| Mepolizumab | Antineoplastic and Immunomodulating | US2008134721 |
| | Agents, Immunosuppressive Agents, | |
| | Interleukin Inhibitors | |
| Ixekizumab | | |
| Insulin Pork | Hypoglycemic Agents, Antidiabetic Agents | |
| Insulin Degludec | | |
| Insulin Beef | | |
| Thyroglobulin | Hormone therapy | U.S. Pat. No. 5,099,001 |
| Anthrax immune globulin | Plasma derivative | |
| human | | |
| Anti-inhibitor coagulant | Blood Coagulation Factors, Antihemophilic | |
| complex | Agent | |
| Anti-thymocyte Globulin | Antibody | |
| (Equine) | | |
| Anti-thymocyte Globulin | Antibody | |
| (Rabbit) | | |
| Brodalumab | Antineoplastic and Immunomodulating | |
| | Agents | |
| C1 Esterase Inhibitor | Blood and Blood Forming Organs | |
| (Recombinant) | | |
| Ca kinumab | Antineoplastic and Immunomodulating | |
| | Agents | |
| Chorionic Gonadotropin | Hormones | U.S. Pat. No. 6,706,681 |
| (Human) | | |
| Chorionic Gonadotropin | Hormones | U.S. Pat. No. 5,767,251 |
| (Recombi nt) | | |
| Coagulation factor X | Blood Coagulation Factors | |
| human | | |
| Dinutuximab | Antibody, Immunosuppresive | US20140170155 |
| | agent, Antineoplastic agent | |
| Efmoroctocog alpha | Antihemophilic Factor | |
| Factor IX Complex | Antihemophilic agent | |
| (Human) | | |
| Hepatitis A Vaccine | Vaccine | |
| Human Varicella-Zoster | Antibody | |
| Immune Globulin | | |
| Ibritumomab tiuxetan | Antibody, Immunosuppressive Agents | CA2149329 |
| Lenograstim | Antineoplastic and Immunomodulating | |
| | Agents | |
| Pegloticase | Enzymes | |
| Protamine sulfate | Heparin Antagonists, Hematologic Agents | |
| Protein S human | Anticoagulant plasma protein | |
| Sipuleucel-T | Antineoplastic and | U.S. Pat. No. 8,153,120 |
| | Immunomodulating Agents | |
| Somatropin recombi nt | Hormones, Hormone Substitutes, | CA1326439, CA2252535, |
| | and Hormone Antagonists | U.S. Pat. No. 5,288,703, |
| | | U.S. Pat. No. 5,849,700, |
| | | U.S. Pat. No. 5,849,704, |
| | | U.S. Pat. No. 5,898,030, |
| | | U.S. Pat. No. 6,004,297, |
| | | U.S. Pat. No. 6,152,897, |
| | | U.S. Pat. No. 6,235,004, |
| | | U.S. Pat. No. 6,899,699 |
| Susoctocog alpha | Blood coagulation factors, | |
| | Antihemorrhagics | |
| Thrombomodulin alpha | Anticoagulant agent, Antiplatelet agent | |

TABLE 29
1.1.3.4.2 Exemplary monoclonal antibody therapies.

| mAb | Target | Indication |
|---|---|---|
| Muromonab-CD3 | CD3 | Kidney transplant rejection |
| Abciximab | GPIIb/IIIa | Prevention of blood clots in |
| | | angioplasty |
| Rituximab | CD20 | Non-Hodgkin lymphoma |
| Palivizumab | RSV | Prevention of respiratory |
| | | syncytial virus infection |
| Infliximab | TNFα | Crohn's disease |
| Trastuzumab | HER2 | Breast cancer |
| Alemtuzumab | CD52 | Chronic myeloid leukemia |
| Adalimumab | TNFα | Rheumatoid arthritis |
| Ibritumomab | CD20 | Non-Hodgkin lymphoma |
| tiuxetan | | |
| Omalizumab | IgE | Asthma |
| Cetuximab | EGFR | Colorectal cancer |
| Bevacizumab | VEGF-A | Colorectal cancer |
| Natalizumab | ITGA4 | Multiple sclerosis |
| Panitumumab | EGFR | Colorectal cancer |
| Ranibizumab | VEGF-A | Macular degeneration |
| Eculizumab | CS | Paroxysmal nocturnal |
| | | hemoglobinuria |
| Certolizumab | TNFα | Crohn's disease |
| pegol | | |
| Ustekinumab | IL-12/23 | Psoriasis |
| Canakinumab | IL-1β | Muckle-Wells syndrome |
| Golimumab | TNFα | Rheumatoid and psoriatic |
| | | arthritis, ankylosing spondylitis |
| Ofatumumab | CD20 | Chronic lymphocytic leukemia |
| Tocilizumab | IL-6R | Rheumatoid arthritis |
| Denosumab | RANKL | Bone loss |
| Belimumab | BLyS | Systemic lupus erythematosus |
| Ipilimumab | CTLA-4 | Metastatic melanoma |
| Brentuximab | CD30 | Hodgkin lymphoma, systemic |
| vedotin | | anaplastic large cell lymphoma |
| Pertuzumab | HER2 | Breast Cancer |
| Trastuzumab | HER2 | Breast cancer |
| emtansine | | |
| Raxibacumab | B. anthrasis PA | Anthrax infection |
| Obinutuzumab | CD20 | Chronic lymphocytic leukemia |
| Siltuximab | IL-6 | Castleman disease |
| Ramucirumab | VEGFR2 | Gastric cancer |
| Vedolizumab | α4β7 | Ulcerative colitis, Crohn |
| | integrin | disease |
| Blinatumomab | CD19, CD3 | Acute lymphoblastic leukemia |
| Nivolumab | PD-1 | Melanoma, non-small cell |
| | | lung cancer |
| Pembrolizumab | PD-1 | Melanoma |
| Idarucizumab | Dabigatran | Reversal of dabigatran- |
| | | induced anticoagulation |
| Necitumumab | EGFR | Non-small cell lung cancer |
| Dinutuximab | GD2 | Neuroblastoma |
| Secukinumab | IL-17α | Psoriasis |
| Mepolizumab | IL-5 | Severe eosinophilic asthma |
| Alirocumab | PCSK9 | High cholesterol |
| Evolocumab | PCSK9 | High cholesterol |
| Daratumumab | CD38 | Multiple myeloma |
| Elotuzumab | SLAMF7 | Multiple myeloma |
| Ixekizumab | IL-17α | Psoriasis |
| Reslizumab | IL-5 | Asthma |
| Olaratumab | PDGFRα | Soft tissue sarcoma |
| Bezlotoxumab | Clostridium | Prevention of Clostridium |
| | difficile | difficile infection recurrence |
| | enterotoxin B | |
| Atezolizumab | PD-L1 | Bladder cancer |
| Obiltoxaximab | B. anthrasis PA | Prevention of inhalational |
| | | anthrax |
| Inotuzumab | CD22 | Acute lymphoblastic |
| ozogamicin | | leukemia |
| Brodalumab | IL-17R | Plaque psoriasis |
| Guselkumab | IL-23 p19 | Plaque psoriasis |
| Dupilumab | IL-4Rα | Atopic dermatitis |
| Sarilumab | IL-6R | Rheumatoid arthritis |
| Avelumab | PD-L1 | Merkel cell carcinoma |
| Ocrelizumab | CD20 | Multiple sclerosis |
| Emicizumab | Factor IXa, X | Hemophilia A |
| Benralizumab | IL-5Rα | Asthma |
| Gemtuzumab | CD33 | Acute myeloid leukemia |
| ozogamicin | | |
| Durvalumab | PD-L1 | Bladder cancer |
| Burosumab | FGF23 | X-linked hypophosphatemia |
| Lanadelumab | Plasma | Hereditary angioedema |
| | kallikrein | attacks |
| Mogamulizumab | CCR4 | Mycosis fungoides or |
| | | Sézary syndrome |
| Erenumab | CGRPR | Migraine prevention |
| Galcanezumab | CGRP | Migraine prevention |
| Tildrakizumab | IL-23 p19 | Plaque psoriasis |
| Cemiplimab | PD-1 | Cutaneous squamous cell |
| | | carcinoma |
| Emapalumab | IFNγ | Primary hemophagocytic |
| | | lymphohistiocytosis |
| Fremanezumab | CGRP | Migraine prevention |
| Ibalizumab | CD4 | HIV infection |
| Moxetumomab | CD22 | Hairy cell leukemia |
| pasudodox | | |
| Ravulizumab | C5 | Paroxysmal nocturnal |
| | | hemoglobinuria |
| Caplacizumab | von Willebrand | Acquired thrombotic |
| | factor | thrombocytopenia purpura |
| Romosozumab | Sclerostin | Osteoporosis in |
| | | postmenopausal women at |
| | | increased risk of fracture |
| Risankizumab | IL-23 p19 | Plaque psoriasis |
| Polatuzumab | CD79β | Diffuse large B-cell |
| vedotin | | lymphoma |
| Brolucizumab | VEGF-A | Macular degeneration |
| Crizanlizumab | P-selectin | Sickle cell disease |

### 1.2 Plant-modification Methods

Gene Writer systems described herein may be used to modify a plant or a plant part (e.g., leaves, roots, flowers, fruits, or seeds), e.g., to increase the fitness of a plant.

### A. Delivery to a Plant

Provided herein are methods of delivering a Gene Writer system described herein to a plant. Included are methods for delivering a Gene Writer system to a plant by contacting the plant, or part thereof, with a Gene Writer system. The methods are useful for modifying the plant to, e.g., increase the fitness of a plant.

More specifically, in some embodiments, a nucleic acid described herein (e.g., a nucleic acid encoding a GeneWriter) may be encoded in a vector, e.g., inserted adjacent to a plant promoter, e.g., a maize ubiquitin promoter (ZmUBI) in a plant vector (e.g., pHUC411). In some embodiments, the nucleic acids described herein are introduced into a plant (e.g., japonica rice) or part of a plant (e.g., a callus of a plant) via agrobacteria. In some embodiments, the systems and methods described herein can be used in plants by replacing a plant gene (e.g., hygromycin phosphotransferase (HPT)) with a null allele (e.g., containing a base substitution at the start codon). Systems and methods for modifying a plant genome are described in Xu et. al. Development of plant prime-editing systems for precise genome editing, 2020, Plant Communications.

In one aspect, provided herein is a method of increasing the fitness of a plant, the method including delivering to the plant the Gene Writer system described herein (e.g., in an effective amount and duration) to increase the fitness of the plant relative to an untreated plant (e.g., a plant that has not been delivered the Gene Writer system).

An increase in the fitness of the plant as a consequence of delivery of a Gene Writer system can manifest in a number of ways, e.g., thereby resulting in a better production of the plant, for example, an improved yield, improved vigor of the plant or quality of the harvested product from the plant, an improvement in pre- or post-harvest traits deemed desirable for agriculture or horticulture (e.g., taste, appearance, shelf life), or for an improvement of traits that otherwise benefit humans (e.g., decreased allergen production). An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional plant-modifying agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. In some instances, the method is effective to increase yield by about 2×-fold, 5×-fold, 10×-fold, 25×-fold, 50×-fold, 75×-fold, 100×-fold, or more than 100×-fold relative to an untreated plant. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a Gene Writer system can also be measured by other means, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional plant-modifying agents (e.g., plant-modifying agents delivered without PMPs).

Accordingly, provided herein is a method of modifying a plant, the method including delivering to the plant an effective amount of any of the Gene Writer systems provided herein, wherein the method modifies the plant and thereby introduces or increases a beneficial trait in the plant (e.g., by about 1%, 2%, 5%, 10%, 20%, 30%, 40%, 50%, 60%, 70%, 80%, 90%, 100%, or more than 100%) relative to an untreated plant. In particular, the method may increase the fitness of the plant (e.g., by about 1%, 2%, 5%, 10%, 20%, 30%, 40%, 50%, 60%, 70%, 80%, 90%, 100%, or more than 100%) relative to an untreated plant.

In some instances, the increase in plant fitness is an increase (e.g., by about 1%, 2%, 5%, 10%, 20%, 30%, 40%, 50%, 60%, 70%, 80%, 90%, 100%, or more than 100%) in disease resistance, drought tolerance, heat tolerance, cold tolerance, salt tolerance, metal tolerance, herbicide tolerance, chemical tolerance, water use efficiency, nitrogen utilization, resistance to nitrogen stress, nitrogen fixation, pest resistance, herbivore resistance, pathogen resistance, yield, yield under water-limited conditions, vigor, growth, photosynthetic capability, nutrition, protein content, carbohydrate content, oil content, biomass, shoot length, root length, root architecture, seed weight, or amount of harvestable produce.

In some instances, the increase in fitness is an increase (e.g., by about 1%, 2%, 5%, 10%, 20%, 30%, 40%, 50%, 60%, 70%, 80%, 90%, 100%, or more than 100%) in development, growth, yield, resistance to abiotic stressors, or resistance to biotic stressors. An abiotic stress refers to an environmental stress condition that a plant or a plant part is subjected to that includes, e.g., drought stress, salt stress, heat stress, cold stress, and low nutrient stress. A biotic stress refers to an environmental stress condition that a plant or plant part is subjected to that includes, e.g. nematode stress, insect herbivory stress, fungal pathogen stress, bacterial pathogen stress, or viral pathogen stress. The stress may be temporary, e.g. several hours, several days, several months, or permanent, e.g. for the life of the plant.

In some instances, the increase in plant fitness is an increase (e.g., by about 1%, 2%, 5%, 10%, 20%, 30%, 40%, 50%, 60%, 70%, 80%, 90%, 100%, or more than 100%) in quality of products harvested from the plant. For example, the increase in plant fitness may be an improvement in commercially favorable features (e.g., taste or appearance) of a product harvested from the plant. In other instances, the increase in plant fitness is an increase in shelf-life of a product harvested from the plant (e.g., by about 1%, 2%, 5%, 10%, 20%, 30%, 40%, 50%, 60%, 70%, 80%, 90%, 100%, or more than 100%).

Alternatively, the increase in fitness may be an alteration of a trait that is beneficial to human or animal health, such as a reduction in allergen production. For example, the increase in fitness may be a decrease (e.g., by about 1%, 2%, 5%, 10%, 20%, 30%, 40%, 50%, 60%, 70%, 80%, 90%, 100%, or more than 100%) in production of an allergen (e.g., pollen) that stimulates an immune response in an animal (e.g., human).

The modification of the plant (e.g., increase in fitness) may arise from modification of one or more plant parts. For example, the plant can be modified by contacting leaf, seed, pollen, root, fruit, shoot, flower, cells, protoplasts, or tissue (e.g., meristematic tissue) of the plant. As such, in another aspect, provided herein is a method of increasing the fitness of a plant, the method including contacting pollen of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In yet another aspect, provided herein is a method of increasing the fitness of a plant, the method including contacting a seed of the plant with an effective amount of any of the Gene Writer systems disclosed herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method including contacting a protoplast of the plant with an effective amount of any of the Gene Writer systems described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In a further aspect, provided herein is a method of increasing the fitness of a plant, the method including contacting a plant cell of the plant with an effective amount of any of the Gene Writer system described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting meristematic tissue of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting an embryo of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

### B. Application Methods

A plant described herein can be exposed to any of the Gene Writer system compositions described herein in any suitable manner that permits delivering or administering the composition to the plant. The Gene Writer system may be delivered either alone or in combination with other active (e.g., fertilizing agents) or inactive substances and may be applied by, for example, spraying, injection (e.g., microinjection), through plants, pouring, dipping, in the form of concentrated liquids, gels, solutions, suspensions, sprays, powders, pellets, briquettes, bricks and the like, formulated to deliver an effective concentration of the plant-modifying composition. Amounts and locations for application of the compositions described herein are generally determined by the habitat of the plant, the lifecycle stage at which the plant can be targeted by the plant-modifying composition, the site where the application is to be made, and the physical and functional characteristics of the plant-modifying composition.

In some instances, the composition is sprayed directly onto a plant, e.g., crops, by e.g., backpack spraying, aerial spraying, crop spraying/dusting etc. In instances where the Gene Writer system is delivered to a plant, the plant receiving the Gene Writer system may be at any stage of plant growth. For example, formulated plant-modifying compositions can be applied as a seed-coating or root treatment in early stages of plant growth or as a total plant treatment at later stages of the crop cycle. In some instances, the plant-modifying composition may be applied as a topical agent to a plant.

Further, the Gene Writer system may be applied (e.g., in the soil in which a plant grows, or in the water that is used to water the plant) as a systemic agent that is absorbed and distributed through the tissues of a plant. In some instances, plants or food organisms may be genetically transformed to express the Gene Writer system.

Delayed or continuous release can also be accomplished by coating the Gene Writer system or a composition with the plant-modifying composition(s) with a dissolvable or bioerodable coating layer, such as gelatin, which coating dissolves or erodes in the environment of use, to then make the plant-modifying com Gene Writer system position available, or by dispersing the agent in a dissolvable or erodable matrix. Such continuous release and/or dispensing means devices may be advantageously employed to consistently maintain an effective concentration of one or more of the plant-modifying compositions described herein.

In some instances, the Gene Writer system is delivered to a part of the plant, e.g., a leaf, seed, pollen, root, fruit, shoot, or flower, or a tissue, cell, or protoplast thereof. In some instances, the Gene Writer system is delivered to a cell of the plant. In some instances, the Gene Writer system is delivered to a protoplast of the plant. In some instances, the Gene Writer system is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the Gene Writer system is delivered to a plant embryo.

### C. Plants

A variety of plants can be delivered to or treated with a Gene Writer system described herein. Plants that can be delivered a Gene Writer system (i.e., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple, Arabidopsis, banana, barley, canola, castor bean, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat and vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes (e.g., a vineyard), kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. In some instances, the composition is delivered to pollen of the plant. In some instances, the composition is delivered to a seed of the plant. In some instances, the composition is delivered to a protoplast of the plant. In some instances, the composition is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the composition is delivered to a plant embryo. In some instances, the composition is delivered to a plant cell. The stages of vegetative and reproductive growth are also referred to herein as “adult” or “mature” plants.

In instances where the Gene Writer system is delivered to a plant part, the plant part may be modified by the plant-modifying agent. Alternatively, the Gene Writer system may be distributed to other parts of the plant (e.g., by the plant's circulatory system) that are subsequently modified by the plant-modifying agent.

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequences specified herein (e.g., by gene name in RepBase or by accession number), including in any Table herein, refer to the database entries current as of Mar. 4, 2020. When one gene or protein references a plurality of sequence accession numbers, all of the sequence variants are encompassed.

### EXAMPLES

The invention is further illustrated by the following examples. The examples are provided for illustrative purposes only and are not to be construed as limiting the scope or content of the invention in any way.

### Example 1: Formulation of Lipid Nanoparticles Encapsulating Firefly Luciferase mRNA

In this example, a reporter mRNA encoding firefly luciferase was formulated into lipid nanoparticles comprising different ionizable lipids. Lipid nanoparticle (LNP) components (ionizable lipid, helper lipid, sterol, PEG) were dissolved in 100% ethanol with the lipid component. These were then prepared at molar ratios of 50:10:38.5:1.5 using ionizable lipid LIPIDV004 or LIPIDV005 (Table A1), DSPC, cholesterol, and DMG-PEG 2000, respectively. Firefly Luciferase mRNA-LNPs containing the ionizable lipid LIPIDV003 (Table A1) were prepared at a molar ratio of 45:9:44:2 using LIPIDV003, DSPC, cholesterol, and DMG-PEG 2000, respectively. Firefly luciferase mRNA used in these formulations was produced by in vitro transcription and encoded the Firefly Luciferase protein, further comprising a 5′ cap, 5′ and 3′ UTRs, and a polyA tail. The mRNA was synthesized under standard conditions for T7 RNA polymerase in vitro transcription with co-transcriptional capping, but with the nucleotide triphosphate UTP 100% substituted with N1-methyl-pseudouridine triphosphate in the reaction. Purified mRNA was dissolved in 25 mM sodium citrate, pH 4 to a concentration of 0.1 mg/mL.

Firefly Luciferase mRNA was formulated into LNPs with a lipid amine to RNA phosphate (N:P) molar ratio of 6. The LNPs were formed by microfluidic mixing of the lipid and RNA solutions using a Precision Nanosystems NanoAssemblr™ Benchtop Instrument, using the manufacturer's recommended settings. A 3:1 ratio of aqueous to organic solvent was maintained during mixing using differential flow rates. After mixing, the LNPs were collected and dialyzed in 15 mM Tris, 5% sucrose buffer at 4° C. overnight. The Firefly Luciferase mRNA-LNP formulation was concentrated by centrifugation with Amicon 10 kDa centrifugal filters (Millipore). The resulting mixture was then filtered using a 0.2 m sterile filter. The final LNP was stored at −80° C. until further use.

TABLE A1
Ionizable Lipids used in Example 1 ( Formula (ix), (vii), and (iii))

| | | Molecular | |
|---|---|---|---|
| LIPID ID | Chemical Name | Weight | Structure |
| LIPIDV003 | (9Z,12Z)-3- ((4,4- bis(octyloxy) butanoyl) oxy)-2- ((((3- (diethylamino) propoxy) carbonyl) oxy)methyl) propyl octadeca- 9, 12-dienoate | 852.29 | 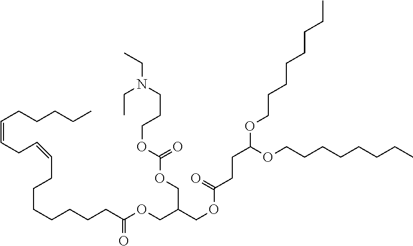 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| LIPIDV004 | Heptadecan- 9-yl 8-((2- hydroxyethyl) (8- (nonyloxy)-8- oxooctyl) amino) octanoate | 710.18 | 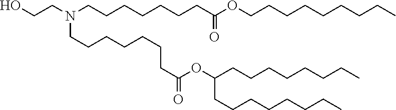 [SMILES: CCCCCCCCCOC(=O)CCCCCCCN(CCO)CCCCCCCC(=O)OC(CCCCCCCC)CCCCCCCC] |
| LIPIDV005 | | 919.56 | 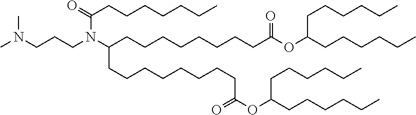 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |

Prepared LNPs were analyzed for size, uniformity, and % RNA encapsulation. The size and uniformity measurements were performed by dynamic light scattering using a Malvern Zetasizer DLS instrument (Malvern Panalytical). LNPs were diluted in PBS prior to being measured by DLS to determine the average particle size (nanometers, nm) and polydispersity index (pdi). The particle sizes of the Firefly Luciferase mRNA-LNPs are shown in Table A2.

TABLE A2
LNP particle size and uniformity

| LNP ID | Ionizable Lipid | Particle Size (nm) | pdi |
|---|---|---|---|
| LNPV019-002 | LIPIDV005 | 77 | 0.04 |
| LNPV006-006 | LIPIDV004 | 71 | 0.08 |
| LNPV011-003 | LIPIDV003 | 87 | 0.08 |

The percent encapsulation of luciferase mRNA was measured by the fluorescence-based RNA quantification assay Ribogreen (ThermoFisher Scientific). LNP samples were diluted in 1×TE buffer and mixed with the Ribogreen reagent per manufacturer's recommendations and measured on a i3 SpectraMax spectrophotomer (Molecular Devices) using 644 nm excitation and 673 nm emission wavelengths. To determine the percent encapsulation, LNPs were measured using the Ribogreen assay with intact LNPs and disrupted LNPs, where the particles were incubated with 1×TE buffer containing 0.2% (w/w) Triton-X100 to disrupt particles to allow encapsulated RNA to interact with the Ribogreen reagent. The samples were again measured on the i3 SpectraMax spectrophotometer to determine the total amount of RNA present. Total RNA was subtracted from the amount of RNA detected when the LNPs were intact to determine the fraction encapsulated. Values were multiplied by 100 to determine the percent encapsulation. The Firefly Luciferase mRNA-LNPs that were measured by Ribogreen and the percent RNA encapsulation is reported in Table A3.

TABLE A3
RNA encapsulation after LNP formulation

| LNP ID | Ionizable Lipid | % mRNA encapsulation |
|---|---|---|
| LNPV019-002 | LIPIDV005 | 98 |
| LNPV006-006 | LIPIDV004 | 92 |
| LNPV011-003 | LIPIDV003 | 97 |

### Example 2: In Vitro Activity Testing of mRNA-LNPs in Primary Hepatocytes

In this example, LNPs comprising the luciferase reporter mRNA were used to deliver the RNA cargo into cells in culture. Primary mouse or primary human hepatocytes were thawed and plated in collagen-coated 96-well tissue culture plates at a density of 30,000 or 50,000 cells per well, respectively. The cells were plated in 1× William's Media E with no phenol red and incubated at 37° C. with 5% CO₂. After 4 hours, the medium was replaced with maintenance medium (lx William's Media E with no phenol containing Hepatocyte Maintenance Supplement Pack (ThermoFisher Scientific)) and cells were grown overnight at 37° C. with 5% CO₂. Firefly Luciferase mRNA-LNPs were thawed at 4° C. and gently mixed. The LNPs were diluted to the appropriate concentration in maintenance media containing 7.5% fetal bovine serum. The LNPs were incubated at 37° C. for 5 minutes prior to being added to the plated primary hepatocytes. To assess delivery of RNA cargo to cells, LNPs were incubated with primary hepatocytes for 24 hours and cells were then harvested and lysed for a Luciferase activity assay. Briefly, medium was aspirated from each well followed by a wash with 1×PBS. The PBS was aspirated from each well and 200 μL passive lysis buffer (PLB) (Promega) was added back to each well and then placed on a plate shaker for 10 minutes. The lysed cells in PLB were frozen and stored at −80° C. until luciferase activity assay was performed.

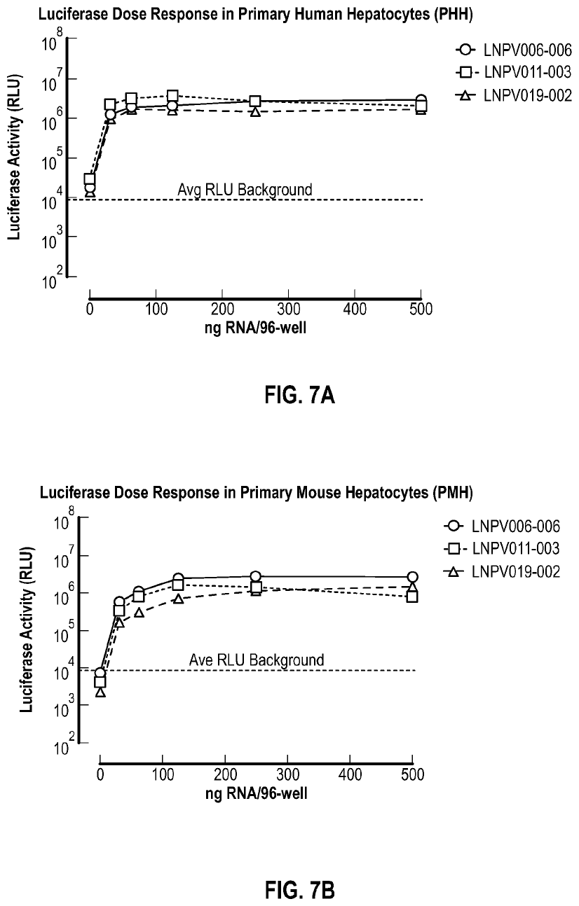

To perform the luciferase activity assay, cellular lysates in passive lysis buffer were thawed, transferred to a round bottom 96-well microtiter plate and spun down at 15,000 g at 4° C. for 3 min to remove cellular debris. The concentration of protein was measured for each sample using the Pierce™ BCA Protein Assay Kit (ThermoFisher Scientific) according to the manufacturer's instructions. Protein concentrations were used to normalize for cell numbers and determine appropriate dilutions of lysates for the luciferase assay. The luciferase activity assay was performed in white-walled 96-well microtiter plates using the luciferase assay reagent (Promega) according to manufacturer's instructions and luminescence was measured using an i3X SpectraMax plate reader (Molecular Devices). The results of the dose-response of Firefly luciferase activity mediated by the Firefly mRNA-LNPs are shown in FIG. 7 and indicate successful LNP-mediated delivery of RNA into primary cells in culture. As shown in FIG. 7A, LNPs formulated as according to Example 1 were analyzed for delivery of cargo to primary human (A) and mouse (B) hepatocytes, as according to Example 2. The luciferase assay revealed dose-responsive luciferase activity from cell lysates, indicating successful delivery of RNA to the cells and expression of Firefly luciferase from the mRNA cargo.

### Example 3: LNP-Mediated Delivery of RNA to the Mouse Liver

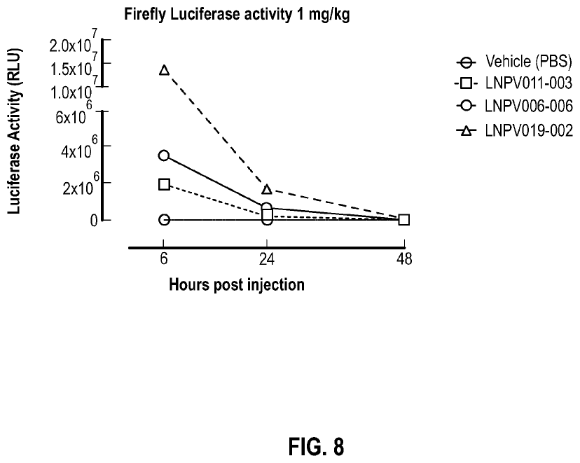

To measure the effectiveness of LNP-mediated delivery of firefly luciferase containing particles to the liver, LNPs were formulated and characterized as described in Example 1 and tested in vitro prior (Example 2) to administration to mice. C57BL/6 male mice (Charles River Labs) at approximately 8 weeks of age were dosed with LNPs via intravenous (i.v.) route at 1 mg/kg. Vehicle control animals were dosed i.v. with 300 μL phosphate buffered saline. Mice were injected via intraperitoneal route with dexamethasone at 5 mg/kg 30 minutes prior to injection of LNPs. Tissues were collected at necropsy at or 6, 24, 48 hours after LNP administration with a group size of 5 mice per time point. Liver and other tissue samples were collected, snap-frozen in liquid nitrogen, and stored at −80° C. until analysis. Frozen liver samples were pulverized on dry ice and transferred to homogenization tubes containing lysing matrix D beads (MP Biomedical). Ice-cold 1× luciferase cell culture lysis reagent (CCLR) (Promega) was added to each tube and the samples were homogenized in a Fast Prep-24 5G Homogenizer (MP Biomedical) at 6 m/s for 40 seconds. The samples were transferred to a clean microcentrifuge tube and clarified by centrifugation. Prior to luciferase activity assay, the protein concentration of liver homogenates was determined for each sample using the Pierce™ BCA Protein Assay Kit (ThermoFisher Scientific) according to the manufacturer's instructions. Luciferase activity was measured with 200 pg (total protein) of liver homogenate using the luciferase assay reagent (Promega) according to manufacturer's instructions using an i3X SpectraMax plate reader (Molecular Devices). Liver samples revealed successful delivery of mRNA by all lipid formulations, with reporter activity following the ranking LIPIDV005>LIPIDV004>LIPIDV003 (FIG. 8). As shown in FIG. 8, Firefly luciferase mRNA-containing LNPs were formulated and delivered to mice by iv, and liver samples were harvested and assayed for luciferase activity at 6, 24, and 48 hours post administration. Reporter activity by the various formulations followed the ranking LIPIDV005>LIPIDV004>LIPIDV003. RNA expression was transient and enzyme levels returned near vehicle background by 48 hours. Post-administration. This assay validated the use of these ionizable lipids and their respective formulations for RNA systems for delivery to the liver.

Without wishing to be limited by example, the lipids and formulations described in this example are support the efficacy for the in vivo delivery of other RNA molecules beyond a reporter mRNA. All-RNA Gene Writing systems can be delivered by the formulations described herein. For example, all-RNA systems employing a Gene Writer polypeptide mRNA, Template RNA, and an optional second-nick gRNA are described for editing the genome in vitro by nucleofection, by using modified nucleotides, by lipofection), and editing cells, e.g., primary T cells. As described in this application, these all-RNA systems have many unique advantages in cellular immunogenicity and toxicity, which is of importance when dealing with more sensitive primary cells, especially immune cells, e.g., T cells, as opposed to immortalized cell culture cell lines. Further, it is contemplated that these all RNA systems could be targeted to alternate tissues and cell types using novel lipid delivery systems as referenced herein, e.g., for delivery to the liver, the lungs, muscle, immune cells, and others, given the function of Gene Writing systems has been validated in multiple cell types in vitro here, and the function of other RNA systems delivered with targeted LNPs is known in the art. The in vivo delivery of Gene Writing systems has potential for great impact in many therapeutic areas, e.g., correcting pathogenic mutations), instilling protective variants, and enhancing cells endogenous to the body, e.g., T cells. Given an appropriate formulation, all-RNA Gene Writing is conceived to enable the manufacture of cell-based therapies in situ in the patient.

### Example 4: Plasmid Delivery of, e.g., MusD

This example demonstrates LTR retrotransposon-mediated integration of a genetic therapeutic payload into the genome of human cells. In order to assess integration, the stability of therapeutic protein expression is measured over time as cells divide. Protein expression stability is conceived to occur as a result of the integration of the therapeutic protein gene expression cassette into the human genome.

To assess expression stability, HEK293T and HepG2 cells are transfected with (1) a template plasmid and an active driver plasmid, (2) a template plasmid and an inactive driver plasmid, or (3) a template plasmid alone. The template plasmid comprises a promoter that mediates transcription of an RNA template which comprises a 5′ LTR, a psi/RRE sequence, a promoter, a CD19-targeted chimeric antigen receptor (CAR) coding sequence, and a 3′ LTR. The driver plasmid comprises a promoter that mediates transcription of a driver RNA that codes for LTR retrotransposon gag proteins (Matrix, Nucleocapsid, and Capsid) and pol proteins (Reverse transcriptase, integrase, and protease). In this example, the 5′ and 3′ LTR of the template RNA, and the gag and pol proteins of the driver RNA are derived from the MusD LTR retrotransposon. The inactive driver plasmid has an inactivating mutation in the reverse transcriptase protein coding sequence, which prevents the reverse transcriptase from reverse transcribing the template RNA.

Beginning three days after transfection, CAR expression is measured via flow cytometry after staining with CD19 antigen fused to FC, with a secondary stain with a fluorophore conjugated to an anti-FC domain (eg as described in doi: 10.3389/fimmu.2020.01770). CAR expression is measured on days 7, 10, 14, 21, 28, and 60 post-transduction. The integration frequency is approximated by determining stable expression of the CAR at day 21, e.g., the fraction of cells that are CAR+ by flow cytometry at day 21. Stability profiles of CAR expression for each system are determined by assaying the frequency (e.g., percent CAR+) and/or the expression level (e.g., median fluorescence signal) of cells at days 28 and 60 post-transduction, as measured by flow cytometry for CAR fluorescence.

In some embodiments, cells treated with a template and active driver plasmid (1) show a decrease in the loss of frequency of CAR expression (e.g., percent CAR+) and/or loss of expression level (e.g., median fluorescence signal) at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold lower than cells treated with (2) and/or (3). In some embodiments, cells treated with a template and active driver plasmid (1) show a higher frequency of expression (e.g., percent CAR+) and/or a higher level of expression (e.g., median fluorescence signal) at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold higher than cells treated with (2) and/or (3).

### Example 5: All-RNA Delivery of, e.g., MusD

This example demonstrates LTR retrotransposon-mediated integration of a genetic payload into the genome of human cells via RNA delivery. In order to assess integration, the stability of therapeutic protein expression is measured over time as cells divide. Protein expression stability is conceived to occur as a result of the integration of a gene expression cassette into the human genome.

To assess expression stability, HEK293T and HepG2 cells are transfected with (1) a template RNA and an active driver mRNA, (2) a template RNA and an inactive driver mRNA, or (3) a template RNA alone. The template RNA comprises a 5′ LTR, a psi/RRE sequence, a promoter, a GFP coding sequence, and a 3′ LTR. The driver mRNA codes for LTR retrotransposon gag proteins (Matrix, Nucleocapsid, and Capsid) and pol proteins (Reverse transcriptase, integrase, and protease). In this example, the 5′ and 3′ LTR of the template RNA, and the gag and pol proteins of the driver RNA are derived from the MusD LTR retrotransposon. The inactive driver mRNA has an inactivating mutation in the reverse transcriptase protein coding sequence, which prevents the reverse transcriptase from reverse transcribing the template RNA.

Beginning three days after transfection, GFP expression is measured via flow cytometry. GFP expression is measured on days 7, 10, 14, 21, 28, and 60 post-transduction. The integration frequency is approximated by determining stable expression of the GFP at day 21, e.g., the fraction of cells that are GFP+ by flow cytometry at day 21. Stability profiles of GFP expression for each system are determined by assaying the frequency (e.g., percent GFP+) and/or the expression level (e.g., median GFP signal) of cells at days 28 and 60 post-transduction, as measured by flow cytometry for GFP fluorescence.

In some embodiments, cells treated with a template RNA and active driver RNA (1) show a decrease in the loss of frequency of GFP expression (e.g., percent GFP+) and/or loss of expression level (e.g., median fluorescence signal) at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold lower than cells treated with (2) and/or (3). In some embodiments, cells treated with a template and active driver plasmid (1) show a higher frequency of expression (e.g., percent GFP+) and/or a higher level of expression (e.g., median fluorescence signal) at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold higher than cells treated with (2) and/or (3).

### Example 6: RNA Delivery of Integration-Deficient LTR

This example demonstrates LTR retrotransposon-mediated establishment of a genetic payload episome in human cells via RNA delivery. In order to assess integration, the stability of therapeutic protein expression is measured over time after RNA delivery. Protein expression stability is conceived to occur as a result of the formation of a DNA episome in the nucleus.

To assess expression stability, unstimulated non-dividing T cells are transfected with (1) a template RNA and an active driver mRNA, (2) a template RNA and an inactive driver mRNA, or (3) a template RNA alone. The template RNA comprises a 5′ LTR, a psi/RRE sequence, a promoter, a GFP coding sequence, and a 3′ LTR. The driver mRNA codes for LTR retrotransposon gag proteins (Matrix, Nucleocapsid, and Capsid) and pol proteins (Reverse transcriptase, integrase, and protease), wherein the integrase protein has a mutation that inactivates integrase functionality (discussed elsewhere herein), resulting in the template RNA being reverse transcribed but not integrated into the genome. In this example, the 5′ and 3′ LTR of the template RNA, and the gag and pol proteins of the driver RNA are derived from the MusD LTR retrotransposon. The inactive driver mRNA further comprises an inactivating mutation in the reverse transcriptase protein coding sequence, which prevents the reverse transcriptase from reverse transcribing the template RNA.

Beginning 6 hours after transfection, GFP expression is measured via flow cytometry. GFP expression is measured at 6 hours, 12 hours, 16 hours, 24 hours, 36 hours, 48 hours, and then on days 3 and day 7 post-transduction. Episomal DNA formation is approximated by determining stable expression of GFP at day 3, e.g., the fraction of cells that are GFP+ by flow cytometry at day 3. Stability profiles of GFP expression for each system are determined by assaying the frequency (e.g., percent GFP+) and/or the expression level (e.g., median GFP signal) of cells 48 hours and 3 days post-transduction, as measured by flow cytometry for GFP fluorescence.

In some embodiments, cells treated with a template RNA and active driver RNA (1) show a decrease in the loss of frequency of GFP expression (e.g., percent GFP+) and/or loss of expression level (e.g., median fluorescence signal) at day 48 and/or day 3 and 7 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold lower than cells treated with (2) and/or (3). In some embodiments, cells treated with a template and active driver plasmid (1) show a higher frequency of expression (e.g., percent GFP+) and/or a higher level of expression (e.g., median fluorescence signal) at 48 hours and/or day 3 and 7 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold higher than cells treated with (2) and/or (3).

Subsequently, the T cells that were transfected with template and active driver RNA are stimulated to divide with T cell activation reagents known in the art. GFP expression is further measured 1, 3, and 7 days post T cell stimulation. The percent of GFP positive cells and median GFP expression decreases at day 3 and 7 relative to day 1. This demonstrates that the episomal DNA is not integrated into the genome.

### Example 7: Plasmid Delivery of LTR Retrotransposons in Trans

This example demonstrates LTR retrotransposon-mediated integration of a genetic payload into the genome of human cells in a trans configuration. In order to assess integration, the stability of therapeutic protein expression was measured over a period of time as cells divide. Protein expression stability was conceived to occur as a result of the integration of the gene expression cassette into the human genome.

To assess expression stability, HEK293T cells were transfected with (1) a template plasmid and an active driver plasmid, (2) a template plasmid and an inactive driver plasmid, or (3) a template plasmid alone. The template plasmid comprised a promoter that mediates transcription of an RNA template which comprised a promoter, an R sequence, a U5 sequence, a primer binding site (PBS) sequence, a heterologous object sequence, polypurine tract (PPT), a 3′ LTR and an SV40 polyA sequence. The 3′LTR comprised a U3, R and U5 sequence. The driver plasmid comprised a promoter that mediates transcription of a driver RNA that codes for LTR retrotransposon gag proteins (Matrix, Nucleocapsid, and Capsid), protease (pro) proteins, and pol proteins (Reverse transcriptase and integrase). In this example, the 5′ R/U5 and 3′ LTR of the template RNA, and the gag, pro and pol proteins of the driver RNA were derived from the MusD6 LTR retrotransposon. In some instances, the 5′ R/U5 and 3′ LTR of the template RNA was derived from the ETnII-B3 retrotransposon. The inactive driver plasmid had an inactivating deletion of the reverse transcriptase protein coding sequence, which prevents the reverse transcriptase from reverse transcribing the template RNA.

A more detailed description of exemplary driver and template configurations are provided below. Sequences used in the exemplary driver and template constructs are listed in Tables S1-S5.

TABLE S1
Common LTR construct elements

| | | SEQ | | | SEQ |
|---|---|---|---|---|---|
| | | ID | | | ID |
| name | nucleic_acid_sequence | NO: | length | protein_sequence | NO: |
| CMV promoter | CGTTACATAACTTACGGTAAATGGCCCGCCTGGCTGACCGCC | 284 | 525 | N/A | |
| (full length for | CAACGACCCCCGCCCATTGACGTCAATAATGACGTATGTTCC | | | | |
| CMV MusD/ETnII | CATAGTAACGCCAATAGGGACTTTCCATTGACGTCAATGGGT | | | | |
| v1, MusD/ETnII | GGAGTATTTACGGTAAACTGCCCACTTGGCAGTACATCAAGT | | | | |
| v2 and IAP v1 | GTATCATATGCCAAGTACGCCCCCTATTGACGTCAATGACGG | | | | |
| designs) | TAAATGGCCCGCCTGGCATTATGCCCAGTACATGACCTTATG | | | | |
| | GGACTTTCCTACTTGGCAGTACATCTACGTATTAGTCATCGCT | | | | |
| | ATTACCATGGTGATGCGGTTTTGGCAGTACATCAATGGGCGT | | | | |
| | GGATAGCGGTTTGACTCACGGGGATTTCCAAGTCTCCACCCC | | | | |
| | ATTGACGTCAATGGGAGTTTGTTTTGGCACCAAAATCAACGG | | | | |
| | GACTTTCCAAAATGTCGTAACAACTCCGCCCCATTGACGCAA | | | | |
| | ATGGGCGGTAGGCGTGTACGGTGGGAGGTCTATATAAGCAG | | | | |
| | AGCTCGTTTAGTGAACCGTCA | | | | |
| CMV promoter | CGTTACATAACTTACGGTAAATGGCCCGCCTGGCTGACCGCC | 285 | 508 | N/A | |
| (3′ truncated | CAACGACCCCCGCCCATTGACGTCAATAATGACGTATGTTCC | | | | |
| for | CATAGTAACGCCAATAGGGACTTTCCATTGACGTCAATGGGT | | | | |
| CMV MusD/ETnII | GGAGTATTTACGGTAAACTGCCCACTTGGCAGTACATCAAGT | | | | |
| v3 and IAP v2 | GTATCATATGCCAAGTACGCCCCCTATTGACGTCAATGACGG | | | | |
| designs) | TAAATGGCCCGCCTGGCATTATGCCCAGTACATGACCTTATG | | | | |
| | GGACTTTCCTACTTGGCAGTACATCTACGTATTAGTCATCGCT | | | | |
| | ATTACCATGGTGATGCGGTTTTGGCAGTACATCAATGGGCGT | | | | |
| | GGATAGCGGTTTGACTCACGGGGATTTCCAAGTCTCCACCCC | | | | |
| | ATTGACGTCAATGGGAGTTTGTTTTGGCACCAAAATCAACGG | | | | |
| | GACTTTCCAAAATGTCGTAACAACTCCGCCCCATTGACGCAA | | | | |
| | ATGGGCGGTAGGCGTGTACGGTGGGAGGTCTATATAAGCAG | | | | |
| | AGCT | | | | |
| SV40 polyA | GATCCAGACATGATAAGATACATTGATGAGTTTGGACAAACC | 286 | 135 | N /A | |
| | ACAACTAGAATGCAGTGAAAAAAATGCTTTATTTGTGAAATT | | | | |
| | TGTGATGCTATTGCTTTATTTGTAACCATTATAAGCTGCAATA | | | | |
| | AACAAGTT | | | | |
| EF1 alpha | GGGCAGAGCGCACATCGCCCACAGTCCCCGAGAAGTTGGGG | 287 | 212 | N/A | |
| promoter (used | GGAGGGGTCGGCAATTGATCCGGTGCCTAGAGAAGGTGGC | | | | |
| in heterologous | GCGGGGTAAACTGGGAAAGTGATGTCGTGTACTGGCTCCGC | | | | |
| object sequence) | CTTTTTCCCGAGGGTGGGGGAGAACCGTATATAAGTGCAGT | | | | |
| | AGTCGCCGTGAACGTTCTTTTTCGCAACGGGTTTGCCGCCAG | | | | |
| | AACACAG | | | | |
| Kozak sequence | GACCCAAGCTTGGCATTCCGGTACTGTTGGTAAAGCCACC | 288 | 40 | N/A | |
| (used in | | | | | |
| heterologous | | | | | |
| object sequence) | | | | | |
| Kozak sequence | GCCGCCACC | | 9 | N/A | |
| (used in compact | | | | | |
| drivers) | | | | | |
| GFP (used in | ATGGTGAGCAAGGGCGAGGAGCTGTTCACCGGGGTGGTGC | 289 | 720 | MVSKGEELFTGVVPILVELDGDVNGHK | 295 |
| heterologous | CCATCCTGGTCGAGCTGGACGGCGACGTAAACGGCCACAAG | | | FSVSGEGEGDATYGKLTLKFICTTGKLPV | |
| object sequence) | TTCAGCGTGTCCGGCGAGGGCGAGGGCGATGCCACCTACGG | | | PWPTLVTTLTYGVQCFSRYPDHMKQH | |
| | CAAGCTGACCCTGAAGTTCATCTGCACCACCGGCAAGCTGCC | | | DFFKSAMPEGYVQERTIFFKDDGNYKT | |
| | CGTGCCCTGGCCCACCCTCGTGACCACCCTGACCTACGGCGT | | | RAEVKFEGDTLVNRIELKGIDFKEDGNIL | |
| | GCAGTGCTTCAGCCGCTACCCCGACCACATGAAGCAGCACGA | | | GHKLEYNYNSHNVYIMADKQKNGIKV | |
| | CTTCTTCAAGTCCGCCATGCCCGAAGGCTACGTCCAGGAGCG | | | NFKIRHNIEDGSVQLADHYQQNTPIGD | |
| | CACCATCTTCTTCAAGGACGACGGCAACTACAAGACCCGCGC | | | GPVLLPDNHYLSTQSALSKDPNEKRDH | |
| | CGAGGTGAAGTTCGAGGGCGACACCCTGGTGAACCGCATCG | | | MVLLEFVTAAGITLGMDELYK* | |
| | AGCTGAAGGGCATCGACTTCAAGGAGGACGGCAACATCCTG | | | | |
| | GGGCACAAGCTGGAGTACAACTACAACAGCCACAACGTCTAT | | | | |
| | ATCATGGCCGACAAGCAGAAGAACGGCATCAAGGTGAACTT | | | | |
| | CAAGATCCGCCACAACATCGAGGACGGCAGCGTGCAGCTCG | | | | |
| | CCGACCACTACCAGCAGAACACCCCCATCGGCGACGGCCCCG | | | | |
| | TGCTGCTGCCCGACAACCACTACCTGAGCACCCAGTCCGCCC | | | | |
| | TGAGCAAAGACCCCAACGAGAAGCGCGATCACATGGTCCTG | | | | |
| | CTGGAGTTCGTGACCGCCGCCGGGATCACTCTCGGCATGGAC | | | | |
| | GAGCTGTACAAGTAA | | | | |
| SplitGFP (used | ATGGTGAGCAAGGGCGAGGAGCTGTTCACCGGGGTGGTGC | 290 | 853 | N/A | |
| in heterologous | CCATCCTGGTCGAGCTGGACGGCGACGTAAACGGCCACAAG | | | | |
| object sequence) | TTCAGCGTGTCCGGCGAGGGCGAGGGCGATGCCACCTACGG | | | | |
| | CAAGCTGACCCTGAAGTTCATCTGCACCACCGGCAAGCTGCC | | | | |
| | CGTGCCCTGGCCCACCCTCGTGACCACCCTGACCTACGGCGT | | | | |
| | GCAGTGCTTCAGCCGCTACCCCGACCACATGAAGCAGCACGA | | | | |
| | CTTCTTCAAGTCCGCCATGCCCGAAGGCTACGTCCAGGAGCG | | | | |
| | CACCATCTTCTTCAAGGACGACGGCAACTACAAGACCCGCGC | | | | |
| | CGAGGTGAAGTTCGAGGGCGACACCCTGGTGAACCGCATCG | | | | |
| | AGCTGAAGGGCATCGACTTCAAGGAGGACGGCAACATCCTG | | | | |
| | GGGCACAAGCTGGAGTACAACTACAACAGCCACAACGTCTAT | | | | |
| | ATCATGGCCGACAAGCAGAAGAACGGCATCAAGGTGAACTT | | | | |
| | CAAGATCCGCCACAACATCGAGGACGGCAGCGTGCAGCTCG | | | | |
| | CCGACCACTACCAGCAGAACACCCCCATCGGCGACGGCCCCG | | | | |
| | TGCTGCTGCCCGACAACCACTACCTGTGGAGAGAAAGGCAA | | | | |
| | AGTGGATGTCAGTAAGACCAATAGGTGCCTATCAGAAACGC | | | | |
| | AAGAGTCTTCTCTGTCTCGACAAGCCCAGTTTCTATTGGTCTC | | | | |
| | CTTAAACCTGTCTTGTAACCTTGATACTTACCTGAGCACCCAG | | | | |
| | TCCGCCCTGAGCAAAGACCCCAACGAGAAGCGCGATCACAT | | | | |
| | GGTCCTGCTGGAGTTCGTGACCGCCGCCGGGATCACTCTCGG | | | | |
| | CATGGACGAGCTGTACAAGTAA | | | | |
| Intron (used in | GTAAGTATCAAGGTTACAAGACAGGTTTAAGGAGACCAATA | 291 | 133 | N/A | |
| within split GFP | GAAACTGGGCTTGTCGAGACAGAGAAGACTCTTGCGTTTCTG | | | | |
| in the antisense | ATAGGCACCTATTGGTCTTACTGACATCCACTTTGCCTTTCTCT | | | | |
| direction) | CCACAG | | | | |
| TK polyA (used | CGGCAATAAAAAGACAGAATAAAACGCACGGGTGTTGGGTC | 292 | 49 | N/A | |
| in heterologous | GTTTGTTC | | | | |
| object sequence) | | | | | |
| EF1 | GGGCAGAGCGCACATCGCCCACAGTCCCCGAGAAGTTGGGG | 293 | 1168 | N/A | |
| alpha + | GGAGGGGTCGGCAATTGATCCGGTGCCTAGAGAAGGTGGC | | | | |
| SplitGFP + | GCGGGGTAAACTGGGAAAGTGATGTCGTGTACTGGCTCCGC | | | | |
| TKpolyA | CTTTTTCCCGAGGGTGGGGGAGAACCGTATATAAGTGCAGT | | | | |
| (heterologous | AGTCGCCGTGAACGTTCTTTTTCGCAACGGGTTTGCCGCCAG | | | | |
| object sequence) | AACACAGGACCCAAGCTTGGCATTCCGGTACTGTTGGTAAAG | | | | |
| | CCACCATGGTGAGCAAGGGCGAGGAGCTGTTCACCGGGGTG | | | | |
| | GTGCCCATCCTGGTCGAGCTGGACGGCGACGTAAACGGCCA | | | | |
| | CAAGTTCAGCGTGTCCGGCGAGGGCGAGGGCGATGCCACCT | | | | |
| | ACGGCAAGCTGACCCTGAAGTTCATCTGCACCACCGGCAAGC | | | | |
| | TGCCCGTGCCCTGGCCCACCCTCGTGACCACCCTGACCTACG | | | | |
| | GCGTGCAGTGCTTCAGCCGCTACCCCGACCACATGAAGCAGC | | | | |
| | ACGACTTCTTCAAGTCCGCCATGCCCGAAGGCTACGTCCAGG | | | | |
| | AGCGCACCATCTTCTTCAAGGACGACGGCAACTACAAGACCC | | | | |
| | GCGCCGAGGTGAAGTTCGAGGGCGACACCCTGGTGAACCGC | | | | |
| | ATCGAGCTGAAGGGCATCGACTTCAAGGAGGACGGCAACAT | | | | |
| | CCTGGGGCACAAGCTGGAGTACAACTACAACAGCCACAACG | | | | |
| | TCTATATCATGGCCGACAAGCAGAAGAACGGCATCAAGGTG | | | | |
| | AACTTCAAGATCCGCCACAACATCGAGGACGGCAGCGTGCA | | | | |
| | GCTCGCCGACCACTACCAGCAGAACACCCCCATCGGCGACG | | | | |
| | GCCCCGTGCTGCTGCCCGACAACCACTACCTGTGGAGAGAAA | | | | |
| | GGCAAAGTGGATGTCAGTAAGACCAATAGGTGCCTATCAGA | | | | |
| | AACGCAAGAGTCTTCTCTGTCTCGACAAGCCCAGTTTCTATTG | | | | |
| | GTCTCCTTAAACCTGTCTTGTAACCTTGATACTTACCTGAGCA | | | | |
| | CCCAGTCCGCCCTGAGCAAAGACCCCAACGAGAAGCGCGAT | | | | |
| | CACATGGTCCTGCTGGAGTTCGTGACCGCCGCCGGGATCACT | | | | |
| | CTCGGCATGGACGAGCTGTACAAGTAAAGCGGCCGGGGGAT | | | | |
| | CGGCAATAAAAAGACAGAATAAAACGCACGGGTGTTGGGTC | | | | |
| | GTTTGTTC | | | | |
| EF1 | GGGCAGAGCGCACATCGCCCACAGTCCCCGAGAAGTTGGGG | 294 | 1035 | N/A | |
| alpha + GFP + | GGAGGGGTCGGCAATTGATCCGGTGCCTAGAGAAGGTGGC | | | | |
| TKpolyA | GCGGGGTAAACTGGGAAAGTGATGTCGTGTACTGGCTCCGC | | | | |
| (heterologous | CTTTTTCCCGAGGGTGGGGGAGAACCGTATATAAGTGCAGT | | | | |
| object sequence) | AGTCGCCGTGAACGTTCTTTTTCGCAACGGGTTTGCCGCCAG | | | | |
| | AACACAGGACCCAAGCTTGGCATTCCGGTACTGTTGGTAAAG | | | | |
| | CCACCATGGTGAGCAAGGGCGAGGAGCTGTTCACCGGGGTG | | | | |
| | GTGCCCATCCTGGTCGAGCTGGACGGCGACGTAAACGGCCA | | | | |
| | CAAGTTCAGCGTGTCCGGCGAGGGCGAGGGCGATGCCACCT | | | | |
| | ACGGCAAGCTGACCCTGAAGTTCATCTGCACCACCGGCAAGC | | | | |
| | TGCCCGTGCCCTGGCCCACCCTCGTGACCACCCTGACCTACG | | | | |
| | GCGTGCAGTGCTTCAGCCGCTACCCCGACCACATGAAGCAGC | | | | |
| | ACGACTTCTTCAAGTCCGCCATGCCCGAAGGCTACGTCCAGG | | | | |
| | AGCGCACCATCTTCTTCAAGGACGACGGCAACTACAAGACCC | | | | |
| | GCGCCGAGGTGAAGTTCGAGGGCGACACCCTGGTGAACCGC | | | | |
| | ATCGAGCTGAAGGGCATCGACTTCAAGGAGGACGGCAACAT | | | | |
| | CCTGGGGCACAAGCTGGAGTACAACTACAACAGCCACAACG | | | | |
| | TCTATATCATGGCCGACAAGCAGAAGAACGGCATCAAGGTG | | | | |
| | AACTTCAAGATCCGCCACAACATCGAGGACGGCAGCGTGCA | | | | |
| | GCTCGCCGACCACTACCAGCAGAACACCCCCATCGGCGACG | | | | |
| | GCCCCGTGCTGCTGCCCGACAACCACTACCTGAGCACCCAGT | | | | |
| | CCGCCCTGAGCAAAGACCCCAACGAGAAGCGCGATCACATG | | | | |
| | GTCCTGCTGGAGTTCGTGACCGCCGCCGGGATCACTCTCGGC | | | | |
| | ATGGACGAGCTGTACAAGTAAAGCGGCCGGGGGATCGGCA | | | | |
| | ATAAAAAGACAGAATAAAACGCACGGGTGTTGGGTCGTTTG | | | | |
| | TTC | | | | |

TABLE S2
MusD LTR retrotransposon driver and template construct elements

| | | | SEQ | | SEQ |
|---|---|---|---|---|---|
| | plasmid_ | | ID | | ID |
| Name | id | nucleic_acid_sequence | NO: | protein_sequence | NO: |
| MusD6 U3 | N/A | TGTAGTCTCCCCTCCCCCAGCCTGAAACCTGCTTGCTCAGGGGTGGAG | 296 | N/A | |
| | | CTTCCCGCTCATCGCTCTGCCACGCCCACTGCTGGAACCTGCGGAGCC | | | |
| | | ACACACGTGCACCTTTCTACTGGACCAGAGATTATTCGGCGGGAATC | | | |
| | | GGGTCCCCTCCCCCTTCCTTCATAACTAGTGTCCCAACAATAAAATTT | | | |
| MusD6 R | N/A | GAGCTTTGATCA | 297 | N/A | |
| (full | | | | | |
| length for | | | | | |
| CMV v1 | | | | | |
| and v3 | | | | | |
| designs) | | | | | |
| MusD6 R | N/A | GCTTTGATCA | 298 | N/A | |
| (5′ | | | | | |
| truncated | | | | | |
| for CMV | | | | | |
| v2 | | | | | |
| designs) | | | | | |
| MusD6 U5 | N/A | GAATGAATTTGTCTTGGCTCCGTTTCTTCTTTCGCCCCGTCTAGATTCC | 299 | N/A | |
| | | TCTCTTACAGCTCGAGTGGCCTTCTCAGTCGAACCGTTCACGTTGCGA | | | |
| | | GCTGCTGGCGGCCGCAACA | | | |
| MusD6 | N/A | TGTAGTCTCCCCTCCCCCAGCCTGAAACCTGCTTGCTCAGGGGTGGAG | 300 | N/A | |
| 5′/3′ LTR | | CTTCCCGCTCATCGCTCTGCCACGCCCACTGCTGGAACCTGCGGAGCC | | | |
| | | ACACACGTGCACCTTTCTACTGGACCAGAGATTATTCGGCGGGAATC | | | |
| | | GGGTCCCCTCCCCCTTCCTTCATAACTAGTGTCCCAACAATAAAATTTG | | | |
| | | AGCTTTGATCAGAATGAATTTGTCTTGGCTCCGTTTCTTCTTTCGCCCC | | | |
| | | GTCTAGATTCCTCTCTTACAGCTCGAGTGGCCTTCTCAGTCGAACCGT | | | |
| | | TCACGTTGCGAGCTGCTGGCGGCCGCAACA | | | |
| MusD6 | N/A | TTTTGGCGCCAGAACTGGGACCTGAAGAATGGC | 301 | N/A | |
| PBS | | | | | |
| (larger | | | | | |
| annota- | | | | | |
| tion) | | | | | |
| MusD6 | N/A | TGGCGCCAGAACTGGGAC | 302 | N/A | |
| PBS | | | | | |
| (shorter | | | | | |
| annota- | | | | | |
| tion) | | | | | |
| MusD6 | N/A | ATGGATCAGGCGGTTGCCCATAGTTTTCAGGAGTTGTTTCAGGCCAG | 303 | MDQAVAHSFQELFQARGVRLEVQLVKNFLGKIDSCCP | 329 |
| gag | | AGGAGTAAGGCTTGAAGTACAATTAGTAAAAAATTTTTTAGGTAAGA | | WFKEEETLDCGTWEKVGEALKITQADNFTLGLWALIND | |
| | | TAGATAGCTGTTGCCCATGGTTCAAGGAAGAAGAAACACTAGATTGT | | AIKDATSPGLSCPQAELVVSQEECLSERASSEKDLLNSKID | |
| | | GGAACCTGGGAGAAAGTTGGTGAGGCCTTAAAAATCACTCAGGCAG | | KCGNSDEKLIFNKNHSDRGAAHYLNENWSSCESPAQPV | |
| | | ATAATTTTACCCTAGGCCTCTGGGCACTCATAAATGATGCAATAAAAG | | VPTSGGATHRDTRLSELEFEIKLQRLTNELRELKKMSEAE | |
| | | ATGCCACTTCCCCAGGGCTAAGTTGCCCCCAGGCGGAGCTTGTGGTA | | KSNSSVVHQVPLEKVVSQAHGKGQNISNTLAFPVVEVV | |
| | | TCTCAGGAGGAGTGCCTGTCAGAGAGGGCCTCCTCAGAAAAAGATCT | | DQQDTRGRHYQTLDFKLIKELKAAVVQYGPSAPFTQALL | |
| | | TCTTAACTCAAAAATTGATAAATGTGGAAACTCGGATGAAAAACTGA | | DTVVESHLTPLDWKTLSKATLSGGDFLLWDSEWRDASK | |
| | | TTTTTAACAAAAATCACTCAGATAGAGGAGCTGCCCATTACCTTAATG | | KTAASNAQAGNSDWDSNMLLGEGPYEGQTNQIDFPV | |
| | | AGAATTGGTCCTCTTGTGAATCTCCTGCTCAACCTGTAGTCCCCACTTC | | AVYAQIATAARRAWGRLPVKGEIGGSLASIRQSSDEPYQ | |
| | | GGGAGGTGCCACTCATAGGGACACACGACTAAGCGAGTTAGAGTTT | | DFVDRLLISASRILGNPDTGSPFVMQLAYENANAICRAAI | |
| | | GAGATTAAGCTTCAGAGGCTGACTAATGAGCTTCGGGAACTAAAAAA | | QPHKGTTDLAGYVRLCADIGPSCETLQGTHAQAMFSRK | |
| | | GATGTCAGAAGCGGAGAAGAGTAACTCTTCTGTAGTTCACCAGGTGC | | RGNSACFKCGSLDHFRIDCPQNKGAEVRQTGRAPGICP | |
| | | CGCTAGAAAAGGTTGTGAGTCAGGCTCATGGGAAAGGACAGAATAT | | RCGKGRHWAKDCKHKTRVLSRPVPGNEERGQPQAPSY | |
| | | CTCTAATACGCTAGCCTTTCCTGTGGTTGAGGTAGTTGATCAGCAAGA | | SKKTAYGALNLLPSQQDQFLSLSGQTQETQDWTSVPLS | |
| | | TACTAGGGGCAGACATTACCAGACCTTAGATTTCAAGTTGATAAAAG | | MQH* | |
| | | AGTTAAAGGCGGCTGTTGTGCAATATGGCCCTTCAGCCCCATTCACTC | | | |
| | | AAGCATTACTGGACACAGTTGTGGAGTCACACTTAACCCCTTTAGATT | | | |
| | | GGAAGACTCTTTCTAAGGCTACCCTGTCAGGAGGAGATTTTTTGCTTT | | | |
| | | GGGATTCTGAATGGCGAGACGCCAGTAAGAAAACTGCTGCTTCTAAC | | | |
| | | GCTCAGGCTGGTAATTCAGACTGGGATAGCAACATGCTTTTAGGAGA | | | |
| | | GGGCCCTTATGAGGGACAGACAAATCAGATTGATTTTCCCGTTGCAG | | | |
| | | TGTACGCGCAAATTGCGACGGCCGCACGCCGTGCTTGGGGAAGGTT | | | |
| | | GCCAGTCAAAGGAGAGATTGGTGGAAGTTTAGCTAGCATTCGGCAG | | | |
| | | AGTTCTGATGAACCATATCAGGATTTTGTGGACAGGCTATTGATTTCA | | | |
| | | GCTAGTAGAATCCTTGGAAATCCGGACACGGGAAGTCCTTTCGTTAT | | | |
| | | GCAATTGGCTTATGAGAATGCTAACGCAATTTGCCGAGCTGCGATTC | | | |
| | | AACCGCATAAGGGAACGACAGATTTGGCGGGATATGTCCGTCTTTGC | | | |
| | | GCAGACATCGGGCCTTCCTGCGAGACCTTGCAGGGAACCCACGCGCA | | | |
| | | GGCAATGTTCTCTAGGAAACGAGGGAATAGTGCATGCTTTAAATGTG | | | |
| | | GAAGTTTAGATCATTTTAGAATTGATTGTCCTCAGAACAAGGGCGCC | | | |
| | | GAGGTTAGACAAACAGGCCGTGCCCCGGGAATATGTCCCCGATGTG | | | |
| | | GAAAGGGCCGCCACTGGGCGAAAGATTGCAAGCATAAAACGAGGGT | | | |
| | | TTTGAGCCGCCCGGTGCCGGGAAACGAGGAAAGGGGTCAGCCCCAG | | | |
| | | GCCCCAAGTTACTCAAAGAAGACAGCTTATGGGGCTCTAAATCTGCT | | | |
| | | GCCCAGCCAACAAGATCAGTTCTTGAGCTTGTCAGGTCAAACCCAGG | | | |
| | | AAACGCAAGACTGGACCTCTGTTCCACTGTCCATGCAGCATTAA | | | |
| MusD6 | N/A | AACGAGGGTTTTGAGCCGCCCGGTGCCGGGAAACGAGGAAAGGGGT | 304 | NEGFEPPGAGKRGKGSAPGPKLLKEDSLWGSKSAAQPT | 330 |
| pro | | CAGCCCCAGGCCCCAAGTTACTCAAAGAAGACAGCTTATGGGGCTCT | | RSVLELVRSNPGNARLDLCSTVHAALTPEVGVQTLPTGV | |
| | | AAATCTGCTGCCCAGCCAACAAGATCAGTTCTTGAGCTTGTCAGGTCA | | FGPLPVGTCGFLLGRSSSIVEGLQIYPGVISNDYEGEIKIIA | |
| | | AACCCAGGAAACGCAAGACTGGACCTCTGTTCCACTGTCCATGCAGC | | ACPRGAITIPANQKIAQLTLIPLRWSLSKFSKNEEGQINFD | |
| | | ATTAACCCCAGAAGTGGGAGTCCAAACTCTGCCTACCGGAGTCTTTG | | SSGVNWVKSITNQRPNLKLILDGKSFEGLIDTGADVTIIR | |
| | | GACCACTACCTGTAGGAACCTGTGGTTTTCTCTTAGGACGAAGCAGTT | | GQDWPSNWPLSVSLTHLQGIGYASNPKRSSKLLTWRDE | |
| | | CTATTGTAGAAGGCCTGCAGATTTATCCAGGTGTTATAAGTAATGATT | | DGKSGNIQPYVMQNLPVTLWGRDLLSQMGVILCSSKE | |
| | | ATGAGGGAGAAATTAAAATCATAGCCGCTTGCCCTCGTGGTGCTATA | | MVTEQTFRQGPLPDRGLIKKGQKIKTFEDLKPHSNVRGL | |
| | | ACTATACCCGCTAATCAGAAAATTGCTCAACTTACCTTGATCCCCTTGC | | KYFQ* | |
| | | GCTGGTCACTATCTAAATTCTCTAAAAATGAAGAAGGACAGATTAACT | | | |
| | | TTGACTCCTCTGGCGTAAATTGGGTGAAATCTATCACTAATCAGAGAC | | | |
| | | CTAACCTTAAATTGATTCTTGATGGAAAAAGCTTTGAAGGATTAATAG | | | |
| | | ATACCGGGGCCGATGTAACCATTATTAGAGGGCAGGACTGGCCCTCA | | | |
| | | AACTGGCCCCTGTCTGTTTCCTTGACTCACCTTCAAGGAATTGGTTAT | | | |
| | | GCCAGTAACCCAAAACGTAGTTCCAAATTGCTAACCTGGAGAGATGA | | | |
| | | GGATGGAAAATCAGGAAATATTCAGCCGTATGTTATGCAAAATTTGC | | | |
| | | CTGTAACCCTGTGGGGAAGAGATCTGTTGTCACAGATGGGCGTTATC | | | |
| | | CTGTGCAGTTCTAAGGAAATGGTGACTGAACAGACGTTCAGGCAGG | | | |
| | | GACCCCTGCCTGATCGTGGACTAATAAAGAAGGGACAGAAAATTAAG | | | |
| | | ACTTTTGAAGATCTTAAACCCCACTCTAACGTGAGAGGTTTAAAGTAT | | | |
| | | TTTCAGTAG | | | |
| MusD6 | N/A | CGTGAGAGGTTTAAAGTATTTTCAGTAGTGGCCGCTGTCTTGCCTGCA | 305 | RERFKVFSVVAAVLPASHAEKIQWRNDIPVWVDQWSLP | 331 |
| pol | | TCCCACGCCGAAAAAATTCAATGGCGTAATGATATTCCGGTGTGGGT | | KEKIEAASLLVQEQLEAGHLVESHSPWNTPIFIIRKKSGK | |
| | | AGATCAGTGGTCTTTACCTAAAGAGAAAATAGAGGCCGCTTCTCTGCT | | WRLLQDLRKVNETMVLMGTLQPGLPSPVAIPKGYYKIVI | |
| | | AGTGCAGGAGCAGTTAGAAGCAGGACATTTGGTGGAGTCTCATTCTC | | DLKDCFFTIPLHPEDCERFAFSVPSVNFKEPMKRYQWTV | |
| | | CCTGGAATACACCCATTTTCATTATCAGGAAGAAATCGGGAAAATGG | | LPQGMANSPTLCQKFVAKAIQPVRQQWPNIYIIHFTDD | |
| | | AGACTGTTGCAAGATTTAAGAAAGGTTAATGAAACCATGGTACTTAT | | VLMAGKDPQDLLLCYGDLRKALADKGLQIASEKIQTQDP | |
| | | GGGAACTTTACAACCGGGGCTCCCCTCCCCAGTAGCCATTCCTAAGG | | YNYLGFRLTDQAVFHQKIVIRRDNLRTLNDFQKLLGDIN | |
| | | GATACTATAAGATTGTTATAGATTTGAAAGATTGTTTCTTTACCATCCC | | WLRPYLKLTTGELKPLFDILKGSSDPTSPRSLTSEGLLALQL | |
| | | TTTGCATCCAGAGGATTGTGAGAGATTTGCTTTTAGTGTTCCTTCTGT | | VEKAIEEQFVTYIDYSLPLHLLIFNTTHVPTGLLWQKFPIM | |
| | | AAATTTCAAGGAACCCATGAAAAGATATCAATGGACAGTTCTCCCGC | | WIHSRISPKRNILPYHEAVAQMIITGRRQALTYFGKEPDII | |
| | | AGGGGATGGCTAATAGTCCCACCTTATGTCAAAAGTTTGTGGCAAAG | | VQPYSVSQDTWLKQHSTDWLLAQLGFEGTIDSHYPQDR | |
| | | GCAATTCAGCCTGTTAGACAACAATGGCCAAATATTTACATCATTCAT | | LIKFLNVHDMIFPKMTSLQPLNNALLIFTDGSSKGRAGYL | |
| | | TTCACAGATGATGTTTTGATGGCGGGAAAGGACCCCCAAGATTTGCT | | ISNQQVIVETPGLSAQLAELTAVLKVFQSVQEAFNIFTDSL | |
| | | TTTGTGTTATGGAGACTTACGAAAGGCCCTGGCTGATAAGGGATTAC | | YVAQSVPLLETCGTFNFNTPSGSLFSELQNIILARKNPFYI | |
| | | AAATTGCTTCTGAAAAGATACAAACTCAGGATCCTTATAATTATTTGG | | GHIRSHSGLPGPLAEGNNCIDRALIGEALVSDRVALAQR | |
| | | GTTTTAGACTCACTGACCAAGCTGTTTTTCACCAGAAAATTGTTATTCG | | DHERFHLSSHTLRLRHKITKEQARMIVKQCPKCITLSPVP | |
| | | TAGAGATAACTTAAGGACCTTAAATGATTTTCAAAAATTGTTAGGTGA | | HLGVNPRGLMPNHIWQMDITHYAEFGKLKYIHVCIDTC | |
| | | TATAAACTGGCTTCGCCCCTATCTAAAGCTTACTACAGGGGAGTTGAA | | SGFLFASLHTGEASKNVIDHCLQAFNAMGLPKLIKTDNG | |
| | | ACCTTTATTTGATATTCTTAAAGGGAGTTCTGATCCTACTTCCCCTAGA | | PSYSSKNFISFCKEFGIKHKTGIPYNPMGQGIVERAHRTLK | |
| | | TCCCTAACCTCAGAAGGTTTACTGGCCTTACAGCTAGTGGAAAAGGCT | | NWLFKTKEGQLYPPRSPKAHLAFTLFVLNFLHTDIKGQS | |
| | | ATTGAAGAACAGTTTGTCACTTACATAGATTACTCCCTGCCGCTGCAC | | AADRHWHPVTSNSYALVKWKDPLTNEWKGPDPVLIW | |
| | | CTGTTAATTTTTAACACGACTCATGTGCCTACGGGATTGCTATGGCAA | | GRGSVCVFSRDEDGARWLPERLIRQTNTDSDSSGKYHSK | |
| | | AAATTTCCTATAATGTGGATACATTCAAGGATTTCTCCCAAACGTAAT | | D* | |
| | | ATTTTGCCATATCATGAAGCAGTGGCTCAGATGATTATCACTGGAAGA | | | |
| | | AGGCAGGCATTGACTTATTTTGGAAAGGAGCCAGATATCATTGTCCA | | | |
| | | GCCTTACAGCGTGAGTCAGGACACTTGGCTGAAACAGCATAGTACAG | | | |
| | | ATTGGTTGCTTGCACAATTAGGGTTTGAAGGAACTATAGATAGCCACT | | | |
| | | ACCCCCAAGATAGGTTGATAAAATTCTTAAATGTACATGATATGATAT | | | |
| | | TTCCTAAGATGACTTCCTTACAGCCTTTAAATAATGCTCTATTGATTTT | | | |
| | | TACTGATGGCTCCTCTAAAGGGCGAGCTGGATATCTTATTAGTAATCA | | | |
| | | ACAGGTTATCGTAGAGACTCCTGGTCTCTCGGCTCAGCTCGCCGAACT | | | |
| | | AACAGCAGTACTGAAGGTTTTTCAGTCTGTACAGGAGGCTTTTAATAT | | | |
| | | TTTTACTGACAGTTTATATGTTGCTCAGTCAGTACCCTTATTGGAAACC | | | |
| | | TGTGGTACTTTTAACTTCAATACGCCGTCAGGATCTTTATTTTCAGAAT | | | |
| | | TACAAAACATCATTCTCGCCCGGAAAAATCCGTTTTATATTGGCCACA | | | |
| | | TACGGTCTCACTCTGGTCTTCCTGGACCTCTGGCAGAGGGTAATAATT | | | |
| | | GCATTGACAGAGCTCTAATAGGAGAAGCCTTAGTTTCAGATCGGGTT | | | |
| | | GCTTTGGCCCAACGTGATCATGAAAGGTTTCATCTCTCTAGCCATACC | | | |
| | | CTAAGGCTCCGACATAAGATCACCAAGGAGCAAGCGAGAATGATTGT | | | |
| | | AAAACAATGTCCTAAATGTATTACTTTATCTCCAGTGCCGCATCTAGG | | | |
| | | AGTTAATCCTAGAGGCCTTATGCCTAATCATATTTGGCAAATGGATAT | | | |
| | | AACCCATTATGCAGAATTTGGAAAACTAAAATATATACATGTTTGCAT | | | |
| | | TGATACTTGTTCAGGATTTCTCTTTGCTTCTCTGCATACAGGAGAAGCT | | | |
| | | TCAAAAAACGTAATTGATCATTGCCTACAAGCATTTAATGCCATGGGA | | | |
| | | TTACCTAAACTTATTAAGACAGACAATGGGCCATCTTATTCCAGTAAA | | | |
| | | AACTTTATTTCATTCTGTAAAGAATTCGGTATTAAACATAAAACTGGA | | | |
| | | ATTCCTTACAACCCCATGGGACAAGGAATAGTTGAACGTGCTCATCGC | | | |
| | | ACCTTAAAGAATTGGCTCTTTAAGACAAAAGAGGGGCAGCTATATCC | | | |
| | | CCCAAGGTCTCCAAAGGCCCACCTTGCCTTCACCTTATTTGTCCTAAAT | | | |
| | | TTCTTGCACACCGATATCAAGGGCCAGTCTGCAGCGGATCGCCACTG | | | |
| | | GCATCCAGTTACTTCTAATTCTTATGCATTGGTAAAATGGAAGGACCC | | | |
| | | CCTGACTAATGAATGGAAGGGTCCAGATCCAGTTCTAATTTGGGGTA | | | |
| | | GAGGCTCAGTTTGTGTTTTTTCACGAGATGAAGATGGAGCACGGTGG | | | |
| | | CTGCCAGAGAGATTAATTCGTCAGACGAACACAGATTCTGACTCTTCT | | | |
| | | GGTAAGTATCATTCTAAAGACTAA | | | |
| MusD6 5′ | N/A | AGAGAGATGCTAAGAGGAACGCTGCTTTGGAGCTCCACAGGAAAGG | 306 | N/A | |
| flank | | ATCTTCGTATCGGACATCGGAGCAACGGACAGGTACACATGCTAGCG | | | |
| | | CTAGCTTAAAATTTCAGTTTTGTAAAGTGTTGCTGAGGATGTGGTAGG | | | |
| | | ATACGAATTAAGCTTGAATCAGTGCTAACCCAACGCTGGTTCTGCTTG | | | |
| | | GGTCAGCAGCGTGTTAATCGGAACTAGAAACGGAAACAGGCAGGTT | | | |
| | | AGCCGCAGCTTTTTAGGAAGCTGCTTAGGTGGAAGAAGAAAGGGTTT | | | |
| | | AAAGTC | | | |
| MusD6 3′ | N/A | AATTCCTTTTGTGCTTAAAATTCAGCTGAGAGCAACAGCTCTCAAAGC | 307 | N/A | |
| flank | | TGTTCTCCAGCTACTCTCTGAGCCAGCTCCCGACAGGAGGCCGGAGA | | | |
| | | CTAGCCTCAGCTTTACAATTTGCATTTAAATAAAGTACCTAGACTTCCC | | | |
| | | CGAAAAAAGTTCTGCTTTTCTACTTTCTCACTGTCTTTCAAGATTTTGT | | | |
| | | CTTTCAAGCAGGTAAATCAACATTCTCGAGGCGGACCAGCGGATGTG | | | |
| | | CATCCCCGCCCCCCTAGAGCACTCAGGTGGCAGCTGTTATCCCCAGTC | | | |
| | | TCAGGACATTCCAGCATGTGGCCTTCAGTCTGAGTTAAAAATTAGGTT | | | |
| | | TACCCAGAGGACTAGAATAGTAGATATTTCTATATTAATAAAGATTGG | | | |
| | | TTTTTATTTTGATAGACAGGCTTAGCCCCTTAGCTGACCTCTGGCTTTT | | | |
| | | CACCCTTGCTGTTACTGCAAGGTGTCTTTAGCTCAATAAGGCTGTGGA | | | |
| | | AAAAAACAGGGATGAGGAGGAACGGCTCCCAGCTCCTATTTTAGCCA | | | |
| | | CAAATCGTGGTGTTACTAACGACATAATTCTTGCTTAGGCTTTGCTAA | | | |
| | | ATCTGAGGTTGATAATTCTCCTTTAGGAGCTGCACAGCGCTCAGAACT | | | |
| | | GTGCATACTGATTTGTGATGGTACAAATTCAGTATGGGCATCGCTTG | | | |
| | | GTGCAGATGGAGGTACTGCAAGGAAAGGTCCCAGCTTGACCATTTCT | | | |
| | | GAGTTTCCTGTGAGATAAACCCGGTTTGAAAGAGGTTGGTACCAAAT | | | |
| | | TATATATCCCTCGGCTCTACCTCGCCTCCCCAAAAGGTACCAGAGCCA | | | |
| | | CAGGTGTGGATTTTAACAGAATCCACGGGAGGAATCGGGTCCATGTC | | | |
| | | CACCCAAGCCAAGGTTAAAAGCCCACTCATCTACGGATGAGAAAATC | | | |
| | | ATTTGATCACCTCAGTTAAGCGCTGCCTTATTTTAACTTAATTAATAGG | | | |
| | | GGGGAGAGAGATTGGAGACTTACTATTGAAAGGGCAAGCCCTTCACT | | | |
| | | GCCTCCCACCCAAATAAAAAAGCCAATTGGCCTTGTACTACAGAGCTG | | | |
| | | GCCGGACCCCTTATCCCTGTTACCCACCAATCATCCAAAAATGCGGAG | | | |
| | | GAATATCAACTTAGTGTTATTCTTATTATAGTGTATTTCACACTTGTTC | | | |
| | | AGTCAAACTTAGCCAGAGTTCCAACGCCCTACTTAAAATTCAACTAGA | | | |
| | | AAGTTACCTACCAAGTACTAATTAGCATTATAAAGTCAGAGCCTACAG | | | |
| | | CTCCAGGCTTTTCAGTTAGTTGTTTACTAAGATAAGAAAAGACAGTCT | | | |
| | | TAGCCAGATACAGTTTACCATAATAAAAGTTAAAGAATCCCAGGGAA | | | |
| | | GCAAGTTTTTTCTTTTAGCCCTAGATTCCAGGCAGAACTATTGAGCAT | | | |
| | | AGATAATTTTTCCCCC | | | |
| MusD | N/A | TTTTAGCGACTAAACACATCACTGAAGAATCCG | 308 | N/A | |
| PBS* | | | | | |
| (larger | | | | | |
| annota- | | | | | |
| tion) | | | | | |
| MusD | N/A | TAGCGACTAAACACATCA | 309 | N/A | |
| PBS* | | | | | |
| (smaller | | | | | |
| annota- | | | | | |
| tion) | | | | | |
| MusD6 | N/A | TCAGGCCAGCTTTTTCTTTTTTTTTAATTTTGTTAATAAAAGGGAGGAG | 310 | N/A | |
| PPT | | A | | | |
| (larger | | | | | |
| annota- | | | | | |
| tion) | | | | | |
| MusD6 | N/A | AAAAGGGAGGAGA | 311 | N/A | |
| PPT | | | | | |
| (shorter | | | | | |
| annota- | | | | | |
| tion) | | | | | |
| DRIVER_ | PLV10038 | GAATTCGAGCTTGCATGCCTGCAGGTCGTTACATAACTTACGGTAAAT | 312 | N/A | |
| COMPACT_ | | GGCCCGCCTGGCTGACCGCCCAACGACCCCCGCCCATTGACGTCAAT | | | |
| pCMV_ | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| delPol | | AAGTGTATCATATGCCAAGTACGCCCCCTATTGACGTCAATGACGGTA | | | |
| | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGATCGCCGCCACCATGGAT | | | |
| | | CAGGCGGTTGCCCATAGTTTTCAGGAGTTGTTTCAGGCCAGAGGAGT | | | |
| | | AAGGCTTGAAGTACAATTAGTAAAAAATTTTTTAGGTAAGATAGATA | | | |
| | | GCTGTTGCCCATGGTTCAAGGAAGAAGAAACACTAGATTGTGGAACC | | | |
| | | TGGGAGAAAGTTGGTGAGGCCTTAAAAATCACTCAGGCAGATAATTT | | | |
| | | TACCCTAGGCCTCTGGGCACTCATAAATGATGCAATAAAAGATGCCA | | | |
| | | CTTCCCCAGGGCTAAGTTGCCCCCAGGCGGAGCTTGTGGTATCTCAG | | | |
| | | GAGGAGTGCCTGTCAGAGAGGGCCTCCTCAGAAAAAGATCTTCTTAA | | | |
| | | CTCAAAAATTGATAAATGTGGAAACTCGGATGAAAAACTGATTTTTAA | | | |
| | | CAAAAATCACTCAGATAGAGGAGCTGCCCATTACCTTAATGAGAATT | | | |
| | | GGTCCTCTTGTGAATCTCCTGCTCAACCTGTAGTCCCCACTTCGGGAG | | | |
| | | GTGCCACTCATAGGGACACACGACTAAGCGAGTTAGAGTTTGAGATT | | | |
| | | AAGCTTCAGAGGCTGACTAATGAGCTTCGGGAACTAAAAAAGATGTC | | | |
| | | AGAAGCGGAGAAGAGTAACTCTTCTGTAGTTCACCAGGTGCCGCTAG | | | |
| | | AAAAGGTTGTGAGTCAGGCTCATGGGAAAGGACAGAATATCTCTAAT | | | |
| | | ACGCTAGCCTTTCCTGTGGTTGAGGTAGTTGATCAGCAAGATACTAG | | | |
| | | GGGCAGACATTACCAGACCTTAGATTTCAAGTTGATAAAAGAGTTAA | | | |
| | | AGGCGGCTGTTGTGCAATATGGCCCTTCAGCCCCATTCACTCAAGCAT | | | |
| | | TACTGGACACAGTTGTGGAGTCACACTTAACCCCTTTAGATTGGAAGA | | | |
| | | CTCTTTCTAAGGCTACCCTGTCAGGAGGAGATTTTTTGCTTTGGGATT | | | |
| | | CTGAATGGCGAGACGCCAGTAAGAAAACTGCTGCTTCTAACGCTCAG | | | |
| | | GCTGGTAATTCAGACTGGGATAGCAACATGCTTTTAGGAGAGGGCCC | | | |
| | | TTATGAGGGACAGACAAATCAGATTGATTTTCCCGTTGCAGTGTACGC | | | |
| | | GCAAATTGCGACGGCCGCACGCCGTGCTTGGGGAAGGTTGCCAGTC | | | |
| | | AAAGGAGAGATTGGTGGAAGTTTAGCTAGCATTCGGCAGAGTTCTGA | | | |
| | | TGAACCATATCAGGATTTTGTGGACAGGCTATTGATTTCAGCTAGTAG | | | |
| | | AATCCTTGGAAATCCGGACACGGGAAGTCCTTTCGTTATGCAATTGGC | | | |
| | | TTATGAGAATGCTAACGCAATTTGCCGAGCTGCGATTCAACCGCATAA | | | |
| | | GGGAACGACAGATTTGGCGGGATATGTCCGTCTTTGCGCAGACATCG | | | |
| | | GGCCTTCCTGCGAGACCTTGCAGGGAACCCACGCGCAGGCAATGTTC | | | |
| | | TCTAGGAAACGAGGGAATAGTGCATGCTTTAAATGTGGAAGTTTAGA | | | |
| | | TCATTTTAGAATTGATTGTCCTCAGAACAAGGGCGCCGAGGTTAGAC | | | |
| | | AAACAGGCCGTGCCCCGGGAATATGTCCCCGATGTGGAAAGGGCCG | | | |
| | | CCACTGGGCGAAAGATTGCAAGCATAAAACGAGGGTTTTGAGCCGCC | | | |
| | | CGGTGCCGGGAAACGAGGAAAGGGGTCAGCCCCAGGCCCCAAGTTA | | | |
| | | CTCAAAGAAGACAGCTTATGGGGCTCTAAATCTGCTGCCCAGCCAAC | | | |
| | | AAGATCAGTTCTTGAGCTTGTCAGGTCAAACCCAGGAAACGCAAGAC | | | |
| | | TGGACCTCTGTTCCACTGTCCATGCAGCATTAACCCCAGAAGTGGGA | | | |
| | | GTCCAAACTCTGCCTACCGGAGTCTTTGGACCACTACCTGTAGGAACC | | | |
| | | TGTGGTTTTCTCTTAGGACGAAGCAGTTCTATTGTAGAAGGCCTGCAG | | | |
| | | ATTTATCCAGGTGTTATAAGTAATGATTATGAGGGAGAAATTAAAATC | | | |
| | | ATAGCCGCTTGCCCTCGTGGTGCTATAACTATACCCGCTAATCAGAAA | | | |
| | | ATTGCTCAACTTACCTTGATCCCCTTGCGCTGGTCACTATCTAAATTCT | | | |
| | | CTAAAAATGAAGAAGGACAGATTAACTTTGACTCCTCTGGCGTAAATT | | | |
| | | GGGTGAAATCTATCACTAATCAGAGACCTAACCTTAAATTGATTCTTG | | | |
| | | ATGGAAAAAGCTTTGAAGGATTAATAGATACCGGGGCCGATGTAACC | | | |
| | | ATTATTAGAGGGCAGGACTGGCCCTCAAACTGGCCCCTGTCTGTTTCC | | | |
| | | TTGACTCACCTTCAAGGAATTGGTTATGCCAGTAACCCAAAACGTAGT | | | |
| | | TCCAAATTGCTAACCTGGAGAGATGAGGATGGAAAATCAGGAAATAT | | | |
| | | TCAGCCGTATGTTATGCAAAATTTGCCTGTAACCCTGTGGGGAAGAG | | | |
| | | ATCTGTTGTCACAGATGGGCGTTATCCTGTGCAGTTCTAAGGAAATG | | | |
| | | GTGACTGAACAGACGTTCAGGCAGGGACCCCTGCCTGATCGTGGACT | | | |
| | | AATAAAGAAGGGACAGAAAATTAAGACTTTTGAAGATCTTAAACCCC | | | |
| | | ACTCTAACGTGAGAGGTTTAAAGTATTTTCAGTAGTGTAAGCGGCCG | | | |
| | | CAACAGGGGATCCAGACATGATAAGATACATTGATGAGTTTGGACAA | | | |
| | | ACCACAACTAGAATGCAGTGAAAAAAATGCTTTATTTGTGAAATTTGT | | | |
| | | GATGCTATTGCTTTATTTGTAACCATTATAAGCTGCAATAAACAAGTT | | | |
| | | AACAACAACAATTGCATTCATTTTATGTTTCAGGTTCAGGGGGAGGTG | | | |
| | | TGGGAGGTTTTTTCGGATCCTCTAGAGTCGACCTGCAGGCATGCAAG | | | |
| | | CTTGGCGTAATCATGGTCATAGCTGTTTCCTGTGTGAAATTGTTATCC | | | |
| | | GCTCACAATTCCACACAACATACGAGCCGGAAGCATAAAGTGTAAAG | | | |
| | | CCTGGGGTGCCTAATGAGTGAGCTAACTCACATTAATTGCGTTGCGCT | | | |
| | | CACTGCCCGCTTTCCAGTCGGGAAACCTGTCGTGCCAGCTGCATTAAT | | | |
| | | GAATCGGCCAACGCGCGGGGAGAGGCGGTTTGCGTATTGGGCGCTC | | | |
| | | TTCCGCTTCCTCGCTCACTGACTCGCTGCGCTCGGTCGTTCGGCTGCG | | | |
| | | GCGAGCGGTATCAGCTCACTCAAAGGCGGTAATACGGTTATCCACAG | | | |
| | | AATCAGGGGATAACGCAGGAAAGAACATGTGAGCAAAAGGCCAGCA | | | |
| | | AAAGGCCAGGAACCGTAAAAAGGCCGCGTTGCTGGCGTTTTTCCATA | | | |
| | | GGCTCCGCCCCCCTGACGAGCATCACAAAAATCGACGCTCAAGTCAG | | | |
| | | AGGTGGCGAAACCCGACAGGACTATAAAGATACCAGGCGTTTCCCCC | | | |
| | | TGGAAGCTCCCTCGTGCGCTCTCCTGTTCCGACCCTGCCGCTTACCGG | | | |
| | | ATACCTGTCCGCCTTTCTCCCTTCGGGAAGCGTGGCGCTTTCTCATAG | | | |
| | | CTCACGCTGTAGGTATCTCAGTTCGGTGTAGGTCGTTCGCTCCAAGCT | | | |
| | | GGGCTGTGTGCACGAACCCCCCGTTCAGCCCGACCGCTGCGCCTTATC | | | |
| | | CGGTAACTATCGTCTTGAGTCCAACCCGGTAAGACACGACTTATCGCC | | | |
| | | ACTGGCAGCAGCCACTGGTAACAGGATTAGCAGAGCGAGGTATGTA | | | |
| | | GGCGGTGCTACAGAGTTCTTGAAGTGGTGGCCTAACTACGGCTACAC | | | |
| | | TAGAAGGACAGTATTTGGTATCTGCGCTCTGCTGAAGCCAGTTACCTT | | | |
| | | CGGAAAAAGAGTTGGTAGCTCTTGATCCGGCAAACAAACCACCGCTG | | | |
| | | GTAGCGGTGGTTTTTTTGTTTGCAAGCAGCAGATTACGCGCAGAAAA | | | |
| | | AAAGGATCTCAAGAAGATCCTTTGATCTTTTCTACGGGGTCTGACGCT | | | |
| | | CAGTGGAACGAAAACTCACGTTAAGGGATTTTGGTCATGAGATTATC | | | |
| | | AAAAAGGATCTTCACCTAGATCCTTTTAAATTAAAAATGAAGTTTTAA | | | |
| | | ATCAATCTAAAGTATATATGAGTAAACTTGGTCTGACAGTTACCAATG | | | |
| | | CTTAATCAGTGAGGCACCTATCTCAGCGATCTGTCTATTTCGTTCATCC | | | |
| | | ATAGTTGCCTGACTCCCCGTCGTGTAGATAACTACGATACGGGAGGG | | | |
| | | CTTACCATCTGGCCCCAGTGCTGCAATGATACCGCGAGACCCACGCTC | | | |
| | | ACCGGCTCCAGATTTATCAGCAATAAACCAGCCAGCCGGAAGGGCCG | | | |
| | | AGCGCAGAAGTGGTCCTGCAACTTTATCCGCCTCCATCCAGTCTATTA | | | |
| | | ATTGTTGCCGGGAAGCTAGAGTAAGTAGTTCGCCAGTTAATAGTTTG | | | |
| | | CGCAACGTTGTTGCCATTGCTACAGGCATCGTGGTGTCACGCTCGTCG | | | |
| | | TTTGGTATGGCTTCATTCAGCTCCGGTTCCCAACGATCAAGGCGAGTT | | | |
| | | ACATGATCCCCCATGTTGTGCAAAAAAGCGGTTAGCTCCTTCGGTCCT | | | |
| | | CCGATCGTTGTCAGAAGTAAGTTGGCCGCAGTGTTATCACTCATGGTT | | | |
| | | ATGGCAGCACTGCATAATTCTCTTACTGTCATGCCATCCGTAAGATGC | | | |
| | | TTTTCTGTGACTGGTGAGTACTCAACCAAGTCATTCTGAGAATAGTGT | | | |
| | | ATGCGGCGACCGAGTTGCTCTTGCCCGGCGTCAATACGGGATAATAC | | | |
| | | CGCGCCACATAGCAGAACTTTAAAAGTGCTCATCATTGGAAAACGTTC | | | |
| | | TTCGGGGCGAAAACTCTCAAGGATCTTACCGCTGTTGAGATCCAGTTC | | | |
| | | GATGTAACCCACTCGTGCACCCAACTGATCTTCAGCATCTTTTACTTTC | | | |
| | | ACCAGCGTTTCTGGGTGAGCAAAAACAGGAAGGCAAAATGCCGCAA | | | |
| | | AAAAGGGAATAAGGGCGACACGGAAATGTTGAATACTCATACTCTTC | | | |
| | | CTTTTTCAATATTATTGAAGCATTTATCAGGGTTATTGTCTCATGAGCG | | | |
| | | GATACATATTTGAATGTATTTAGAAAAATAAACAAATAGGGGTTCCG | | | |
| | | CGCACATTTCCCCGAAAAGTGCCACCTGACGTCTAAGAAACCATTATT | | | |
| | | ATCATGACATTAACCTATAAAAATAGGCGTATCACGAGGCCCTTTCGT | | | |
| | | CTCGCGCGTTTCGGTGATGACGGTGAAAACCTCTGACACATGCAGCT | | | |
| | | CCCGGAGACGGTCACAGCTTGTCTGTAAGCGGATGCCGGGAGCAGA | | | |
| | | CAAGCCCGTCAGGGCGCGTCAGCGGGTGTTGGCGGGTGTCGGGGCT | | | |
| | | GGCTTAACTATGCGGCATCAGAGCAGATTGTACTGAGAGTGCACCAT | | | |
| | | ATGCGGTGTGAAATACCGCACAGATGCGTAAGGAGAAAATACCGCA | | | |
| | | TCAGGCGCCATTCGCCATTCAGGCTGCGCAACTGTTGGGAAGGGCGA | | | |
| | | TCGGTGCGGGCCTCTTCGCTATTACGCCAGCTGGCGAAAGGGGGATG | | | |
| | | TGCTGCAAGGCGATTAAGTTGGGTAACGCCAGGGTTTTCCCAGTCAC | | | |
| | | GACGTTGTAAAACGACGGCCAGT | | | |
| DRIVER_ | PLV10039 | GAATTCGAGCTTGCATGCCTGCAGGTCGTTACATAACTTACGGTAAAT | 313 | N/A | |
| COMPACT_ | | GGCCCGCCTGGCTGACCGCCCAACGACCCCCGCCCATTGACGTCAAT | | | |
| pCMV_ | | AATGACGTATGTTCCCATAGTAACGCCAATAGGGACTTTCCATTGACG | | | |
| MusD6 | | TCAATGGGTGGAGTATTTACGGTAAACTGCCCACTTGGCAGTACATC | | | |
| | | AAGTGTATCATATGCCAAGTACGCCCCCTATTGACGTCAATGACGGTA | | | |
| | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGATCGCCGCCACCATGGAT | | | |
| | | CAGGCGGTTGCCCATAGTTTTCAGGAGTTGTTTCAGGCCAGAGGAGT | | | |
| | | AAGGCTTGAAGTACAATTAGTAAAAAATTTTTTAGGTAAGATAGATA | | | |
| | | GCTGTTGCCCATGGTTCAAGGAAGAAGAAACACTAGATTGTGGAACC | | | |
| | | TGGGAGAAAGTTGGTGAGGCCTTAAAAATCACTCAGGCAGATAATTT | | | |
| | | TACCCTAGGCCTCTGGGCACTCATAAATGATGCAATAAAAGATGCCA | | | |
| | | CTTCCCCAGGGCTAAGTTGCCCCCAGGCGGAGCTTGTGGTATCTCAG | | | |
| | | GAGGAGTGCCTGTCAGAGAGGGCCTCCTCAGAAAAAGATCTTCTTAA | | | |
| | | CTCAAAAATTGATAAATGTGGAAACTCGGATGAAAAACTGATTTTTAA | | | |
| | | CAAAAATCACTCAGATAGAGGAGCTGCCCATTACCTTAATGAGAATT | | | |
| | | GGTCCTCTTGTGAATCTCCTGCTCAACCTGTAGTCCCCACTTCGGGAG | | | |
| | | GTGCCACTCATAGGGACACACGACTAAGCGAGTTAGAGTTTGAGATT | | | |
| | | AAGCTTCAGAGGCTGACTAATGAGCTTCGGGAACTAAAAAAGATGTC | | | |
| | | AGAAGCGGAGAAGAGTAACTCTTCTGTAGTTCACCAGGTGCCGCTAG | | | |
| | | AAAAGGTTGTGAGTCAGGCTCATGGGAAAGGACAGAATATCTCTAAT | | | |
| | | ACGCTAGCCTTTCCTGTGGTTGAGGTAGTTGATCAGCAAGATACTAG | | | |
| | | GGGCAGACATTACCAGACCTTAGATTTCAAGTTGATAAAAGAGTTAA | | | |
| | | AGGCGGCTGTTGTGCAATATGGCCCTTCAGCCCCATTCACTCAAGCAT | | | |
| | | TACTGGACACAGTTGTGGAGTCACACTTAACCCCTTTAGATTGGAAGA | | | |
| | | CTCTTTCTAAGGCTACCCTGTCAGGAGGAGATTTTTTGCTTTGGGATT | | | |
| | | CTGAATGGCGAGACGCCAGTAAGAAAACTGCTGCTTCTAACGCTCAG | | | |
| | | GCTGGTAATTCAGACTGGGATAGCAACATGCTTTTAGGAGAGGGCCC | | | |
| | | TTATGAGGGACAGACAAATCAGATTGATTTTCCCGTTGCAGTGTACGC | | | |
| | | GCAAATTGCGACGGCCGCACGCCGTGCTTGGGGAAGGTTGCCAGTC | | | |
| | | AAAGGAGAGATTGGTGGAAGTTTAGCTAGCATTCGGCAGAGTTCTGA | | | |
| | | TGAACCATATCAGGATTTTGTGGACAGGCTATTGATTTCAGCTAGTAG | | | |
| | | AATCCTTGGAAATCCGGACACGGGAAGTCCTTTCGTTATGCAATTGGC | | | |
| | | TTATGAGAATGCTAACGCAATTTGCCGAGCTGCGATTCAACCGCATAA | | | |
| | | GGGAACGACAGATTTGGCGGGATATGTCCGTCTTTGCGCAGACATCG | | | |
| | | GGCCTTCCTGCGAGACCTTGCAGGGAACCCACGCGCAGGCAATGTTC | | | |
| | | TCTAGGAAACGAGGGAATAGTGCATGCTTTAAATGTGGAAGTTTAGA | | | |
| | | TCATTTTAGAATTGATTGTCCTCAGAACAAGGGCGCCGAGGTTAGAC | | | |
| | | AAACAGGCCGTGCCCCGGGAATATGTCCCCGATGTGGAAAGGGCCG | | | |
| | | CCACTGGGCGAAAGATTGCAAGCATAAAACGAGGGTTTTGAGCCGCC | | | |
| | | CGGTGCCGGGAAACGAGGAAAGGGGTCAGCCCCAGGCCCCAAGTTA | | | |
| | | CTCAAAGAAGACAGCTTATGGGGCTCTAAATCTGCTGCCCAGCCAAC | | | |
| | | AAGATCAGTTCTTGAGCTTGTCAGGTCAAACCCAGGAAACGCAAGAC | | | |
| | | TGGACCTCTGTTCCACTGTCCATGCAGCATTAACCCCAGAAGTGGGA | | | |
| | | GTCCAAACTCTGCCTACCGGAGTCTTTGGACCACTACCTGTAGGAACC | | | |
| | | TGTGGTTTTCTCTTAGGACGAAGCAGTTCTATTGTAGAAGGCCTGCAG | | | |
| | | ATTTATCCAGGTGTTATAAGTAATGATTATGAGGGAGAAATTAAAATC | | | |
| | | ATAGCCGCTTGCCCTCGTGGTGCTATAACTATACCCGCTAATCAGAAA | | | |
| | | ATTGCTCAACTTACCTTGATCCCCTTGCGCTGGTCACTATCTAAATTCT | | | |
| | | CTAAAAATGAAGAAGGACAGATTAACTTTGACTCCTCTGGCGTAAATT | | | |
| | | GGGTGAAATCTATCACTAATCAGAGACCTAACCTTAAATTGATTCTTG | | | |
| | | ATGGAAAAAGCTTTGAAGGATTAATAGATACCGGGGCCGATGTAACC | | | |
| | | ATTATTAGAGGGCAGGACTGGCCCTCAAACTGGCCCCTGTCTGTTTCC | | | |
| | | TTGACTCACCTTCAAGGAATTGGTTATGCCAGTAACCCAAAACGTAGT | | | |
| | | TCCAAATTGCTAACCTGGAGAGATGAGGATGGAAAATCAGGAAATAT | | | |
| | | TCAGCCGTATGTTATGCAAAATTTGCCTGTAACCCTGTGGGGAAGAG | | | |
| | | ATCTGTTGTCACAGATGGGCGTTATCCTGTGCAGTTCTAAGGAAATG | | | |
| | | GTGACTGAACAGACGTTCAGGCAGGGACCCCTGCCTGATCGTGGACT | | | |
| | | AATAAAGAAGGGACAGAAAATTAAGACTTTTGAAGATCTTAAACCCC | | | |
| | | ACTCTAACGTGAGAGGTTTAAAGTATTTTCAGTAGTGGCCGCTGTCTT | | | |
| | | GCCTGCATCCCACGCCGAAAAAATTCAATGGCGTAATGATATTCCGGT | | | |
| | | GTGGGTAGATCAGTGGTCTTTACCTAAAGAGAAAATAGAGGCCGCTT | | | |
| | | CTCTGCTAGTGCAGGAGCAGTTAGAAGCAGGACATTTGGTGGAGTCT | | | |
| | | CATTCTCCCTGGAATACACCCATTTTCATTATCAGGAAGAAATCGGGA | | | |
| | | AAATGGAGACTGTTGCAAGATTTAAGAAAGGTTAATGAAACCATGGT | | | |
| | | ACTTATGGGAACTTTACAACCGGGGCTCCCCTCCCCAGTAGCCATTCC | | | |
| | | TAAGGGATACTATAAGATTGTTATAGATTTGAAAGATTGTTTCTTTAC | | | |
| | | CATCCCTTTGCATCCAGAGGATTGTGAGAGATTTGCTTTTAGTGTTCC | | | |
| | | TTCTGTAAATTTCAAGGAACCCATGAAAAGATATCAATGGACAGTTCT | | | |
| | | CCCGCAGGGGATGGCTAATAGTCCCACCTTATGTCAAAAGTTTGTGG | | | |
| | | CAAAGGCAATTCAGCCTGTTAGACAACAATGGCCAAATATTTACATCA | | | |
| | | TTCATTTCACAGATGATGTTTTGATGGCGGGAAAGGACCCCCAAGATT | | | |
| | | TGCTTTTGTGTTATGGAGACTTACGAAAGGCCCTGGCTGATAAGGGA | | | |
| | | TTACAAATTGCTTCTGAAAAGATACAAACTCAGGATCCTTATAATTATT | | | |
| | | TGGGTTTTAGACTCACTGACCAAGCTGTTTTTCACCAGAAAATTGTTA | | | |
| | | TTCGTAGAGATAACTTAAGGACCTTAAATGATTTTCAAAAATTGTTAG | | | |
| | | GTGATATAAACTGGCTTCGCCCCTATCTAAAGCTTACTACAGGGGAGT | | | |
| | | TGAAACCTTTATTTGATATTCTTAAAGGGAGTTCTGATCCTACTTCCCC | | | |
| | | TAGATCCCTAACCTCAGAAGGTTTACTGGCCTTACAGCTAGTGGAAAA | | | |
| | | GGCTATTGAAGAACAGTTTGTCACTTACATAGATTACTCCCTGCCGCT | | | |
| | | GCACCTGTTAATTTTTAACACGACTCATGTGCCTACGGGATTGCTATG | | | |
| | | GCAAAAATTTCCTATAATGTGGATACATTCAAGGATTTCTCCCAAACG | | | |
| | | TAATATTTTGCCATATCATGAAGCAGTGGCTCAGATGATTATCACTGG | | | |
| | | AAGAAGGCAGGCATTGACTTATTTTGGAAAGGAGCCAGATATCATTG | | | |
| | | TCCAGCCTTACAGCGTGAGTCAGGACACTTGGCTGAAACAGCATAGT | | | |
| | | ACAGATTGGTTGCTTGCACAATTAGGGTTTGAAGGAACTATAGATAG | | | |
| | | CCACTACCCCCAAGATAGGTTGATAAAATTCTTAAATGTACATGATAT | | | |
| | | GATATTTCCTAAGATGACTTCCTTACAGCCTTTAAATAATGCTCTATTG | | | |
| | | ATTTTTACTGATGGCTCCTCTAAAGGGCGAGCTGGATATCTTATTAGT | | | |
| | | AATCAACAGGTTATCGTAGAGACTCCTGGTCTCTCGGCTCAGCTCGCC | | | |
| | | GAACTAACAGCAGTACTGAAGGTTTTTCAGTCTGTACAGGAGGCTTTT | | | |
| | | AATATTTTTACTGACAGTTTATATGTTGCTCAGTCAGTACCCTTATTGG | | | |
| | | AAACCTGTGGTACTTTTAACTTCAATACGCCGTCAGGATCTTTATTTTC | | | |
| | | AGAATTACAAAACATCATTCTCGCCCGGAAAAATCCGTTTTATATTGG | | | |
| | | CCACATACGGTCTCACTCTGGTCTTCCTGGACCTCTGGCAGAGGGTAA | | | |
| | | TAATTGCATTGACAGAGCTCTAATAGGAGAAGCCTTAGTTTCAGATCG | | | |
| | | GGTTGCTTTGGCCCAACGTGATCATGAAAGGTTTCATCTCTCTAGCCA | | | |
| | | TACCCTAAGGCTCCGACATAAGATCACCAAGGAGCAAGCGAGAATGA | | | |
| | | TTGTAAAACAATGTCCTAAATGTATTACTTTATCTCCAGTGCCGCATCT | | | |
| | | AGGAGTTAATCCTAGAGGCCTTATGCCTAATCATATTTGGCAAATGGA | | | |
| | | TATAACCCATTATGCAGAATTTGGAAAACTAAAATATATACATGTTTG | | | |
| | | CATTGATACTTGTTCAGGATTTCTCTTTGCTTCTCTGCATACAGGAGAA | | | |
| | | GCTTCAAAAAACGTAATTGATCATTGCCTACAAGCATTTAATGCCATG | | | |
| | | GGATTACCTAAACTTATTAAGACAGACAATGGGCCATCTTATTCCAGT | | | |
| | | AAAAACTTTATTTCATTCTGTAAAGAATTCGGTATTAAACATAAAACT | | | |
| | | GGAATTCCTTACAACCCCATGGGACAAGGAATAGTTGAACGTGCTCA | | | |
| | | TCGCACCTTAAAGAATTGGCTCTTTAAGACAAAAGAGGGGCAGCTAT | | | |
| | | ATCCCCCAAGGTCTCCAAAGGCCCACCTTGCCTTCACCTTATTTGTCCT | | | |
| | | AAATTTCTTGCACACCGATATCAAGGGCCAGTCTGCAGCGGATCGCC | | | |
| | | ACTGGCATCCAGTTACTTCTAATTCTTATGCATTGGTAAAATGGAAGG | | | |
| | | ACCCCCTGACTAATGAATGGAAGGGTCCAGATCCAGTTCTAATTTGG | | | |
| | | GGTAGAGGCTCAGTTTGTGTTTTTTCACGAGATGAAGATGGAGCACG | | | |
| | | GTGGCTGCCAGAGAGATTAATTCGTCAGACGAACACAGATTCTGACT | | | |
| | | CTTCTGGTAAGTATCATTCTAAAGACTAAGCGGCCGCAACAGGGGAT | | | |
| | | CCAGACATGATAAGATACATTGATGAGTTTGGACAAACCACAACTAG | | | |
| | | AATGCAGTGAAAAAAATGCTTTATTTGTGAAATTTGTGATGCTATTGC | | | |
| | | TTTATTTGTAACCATTATAAGCTGCAATAAACAAGTTAACAACAACAA | | | |
| | | TTGCATTCATTTTATGTTTCAGGTTCAGGGGGAGGTGTGGGAGGTTTT | | | |
| | | TTCGGATCCTCTAGAGTCGACCTGCAGGCATGCAAGCTTGGCGTAAT | | | |
| | | CATGGTCATAGCTGTTTCCTGTGTGAAATTGTTATCCGCTCACAATTCC | | | |
| | | ACACAACATACGAGCCGGAAGCATAAAGTGTAAAGCCTGGGGTGCC | | | |
| | | TAATGAGTGAGCTAACTCACATTAATTGCGTTGCGCTCACTGCCCGCT | | | |
| | | TTCCAGTCGGGAAACCTGTCGTGCCAGCTGCATTAATGAATCGGCCA | | | |
| | | ACGCGCGGGGAGAGGCGGTTTGCGTATTGGGCGCTCTTCCGCTTCCT | | | |
| | | CGCTCACTGACTCGCTGCGCTCGGTCGTTCGGCTGCGGCGAGCGGTA | | | |
| | | TCAGCTCACTCAAAGGCGGTAATACGGTTATCCACAGAATCAGGGGA | | | |
| | | TAACGCAGGAAAGAACATGTGAGCAAAAGGCCAGCAAAAGGCCAGG | | | |
| | | AACCGTAAAAAGGCCGCGTTGCTGGCGTTTTTCCATAGGCTCCGCCCC | | | |
| | | CCTGACGAGCATCACAAAAATCGACGCTCAAGTCAGAGGTGGCGAA | | | |
| | | ACCCGACAGGACTATAAAGATACCAGGCGTTTCCCCCTGGAAGCTCC | | | |
| | | CTCGTGCGCTCTCCTGTTCCGACCCTGCCGCTTACCGGATACCTGTCC | | | |
| | | GCCTTTCTCCCTTCGGGAAGCGTGGCGCTTTCTCATAGCTCACGCTGT | | | |
| | | AGGTATCTCAGTTCGGTGTAGGTCGTTCGCTCCAAGCTGGGCTGTGT | | | |
| | | GCACGAACCCCCCGTTCAGCCCGACCGCTGCGCCTTATCCGGTAACTA | | | |
| | | TCGTCTTGAGTCCAACCCGGTAAGACACGACTTATCGCCACTGGCAGC | | | |
| | | AGCCACTGGTAACAGGATTAGCAGAGCGAGGTATGTAGGCGGTGCT | | | |
| | | ACAGAGTTCTTGAAGTGGTGGCCTAACTACGGCTACACTAGAAGGAC | | | |
| | | AGTATTTGGTATCTGCGCTCTGCTGAAGCCAGTTACCTTCGGAAAAAG | | | |
| | | AGTTGGTAGCTCTTGATCCGGCAAACAAACCACCGCTGGTAGCGGTG | | | |
| | | GTTTTTTTGTTTGCAAGCAGCAGATTACGCGCAGAAAAAAAGGATCTC | | | |
| | | AAGAAGATCCTTTGATCTTTTCTACGGGGTCTGACGCTCAGTGGAACG | | | |
| | | AAAACTCACGTTAAGGGATTTTGGTCATGAGATTATCAAAAAGGATC | | | |
| | | TTCACCTAGATCCTTTTAAATTAAAAATGAAGTTTTAAATCAATCTAAA | | | |
| | | GTATATATGAGTAAACTTGGTCTGACAGTTACCAATGCTTAATCAGTG | | | |
| | | AGGCACCTATCTCAGCGATCTGTCTATTTCGTTCATCCATAGTTGCCTG | | | |
| | | ACTCCCCGTCGTGTAGATAACTACGATACGGGAGGGCTTACCATCTG | | | |
| | | GCCCCAGTGCTGCAATGATACCGCGAGACCCACGCTCACCGGCTCCA | | | |
| | | GATTTATCAGCAATAAACCAGCCAGCCGGAAGGGCCGAGCGCAGAA | | | |
| | | GTGGTCCTGCAACTTTATCCGCCTCCATCCAGTCTATTAATTGTTGCCG | | | |
| | | GGAAGCTAGAGTAAGTAGTTCGCCAGTTAATAGTTTGCGCAACGTTG | | | |
| | | TTGCCATTGCTACAGGCATCGTGGTGTCACGCTCGTCGTTTGGTATGG | | | |
| | | CTTCATTCAGCTCCGGTTCCCAACGATCAAGGCGAGTTACATGATCCC | | | |
| | | CCATGTTGTGCAAAAAAGCGGTTAGCTCCTTCGGTCCTCCGATCGTTG | | | |
| | | TCAGAAGTAAGTTGGCCGCAGTGTTATCACTCATGGTTATGGCAGCA | | | |
| | | CTGCATAATTCTCTTACTGTCATGCCATCCGTAAGATGCTTTTCTGTGA | | | |
| | | CTGGTGAGTACTCAACCAAGTCATTCTGAGAATAGTGTATGCGGCGA | | | |
| | | CCGAGTTGCTCTTGCCCGGCGTCAATACGGGATAATACCGCGCCACA | | | |
| | | TAGCAGAACTTTAAAAGTGCTCATCATTGGAAAACGTTCTTCGGGGC | | | |
| | | GAAAACTCTCAAGGATCTTACCGCTGTTGAGATCCAGTTCGATGTAAC | | | |
| | | CCACTCGTGCACCCAACTGATCTTCAGCATCTTTTACTTTCACCAGCGT | | | |
| | | TTCTGGGTGAGCAAAAACAGGAAGGCAAAATGCCGCAAAAAAGGGA | | | |
| | | ATAAGGGCGACACGGAAATGTTGAATACTCATACTCTTCCTTTTTCAA | | | |
| | | TATTATTGAAGCATTTATCAGGGTTATTGTCTCATGAGCGGATACATA | | | |
| | | TTTGAATGTATTTAGAAAAATAAACAAATAGGGGTTCCGCGCACATTT | | | |
| | | CCCCGAAAAGTGCCACCTGACGTCTAAGAAACCATTATTATCATGACA | | | |
| | | TTAACCTATAAAAATAGGCGTATCACGAGGCCCTTTCGTCTCGCGCGT | | | |
| | | TTCGGTGATGACGGTGAAAACCTCTGACACATGCAGCTCCCGGAGAC | | | |
| | | GGTCACAGCTTGTCTGTAAGCGGATGCCGGGAGCAGACAAGCCCGTC | | | |
| | | AGGGCGCGTCAGCGGGTGTTGGCGGGTGTCGGGGCTGGCTTAACTA | | | |
| | | TGCGGCATCAGAGCAGATTGTACTGAGAGTGCACCATATGCGGTGTG | | | |
| | | AAATACCGCACAGATGCGTAAGGAGAAAATACCGCATCAGGCGCCAT | | | |
| | | TCGCCATTCAGGCTGCGCAACTGTTGGGAAGGGCGATCGGTGCGGG | | | |
| | | CCTCTTCGCTATTACGCCAGCTGGCGAAAGGGGGATGTGCTGCAAGG | | | |
| | | CGATTAAGTTGGGTAACGCCAGGGTTTTCCCAGTCACGACGTTGTAA | | | |
| | | AACGACGGCCAGT | | | |
| DRIVER_ | PLV10046 | GAATTCGAGCTTGCATGCCTGCAGGTCGTTACATAACTTACGGTAAAT | 314 | N/A | |
| pCMV_R- | | GGCCCGCCTGGCTGACCGCCCAACGACCCCCGCCCATTGACGTCAAT | | | |
| U5- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| LTR_v1 | | AAGTGTATCATATGCCAAGTACGCCCCCTATTGACGTCAATGACGGTA | | | |
| | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGAGCTTTGATCAGAATGAA | | | |
| | | TTTGTCTTGGCTCCGTTTCTTCTTTCGCCCCGTCTAGATTCCTCTCTTAC | | | |
| | | AGCTCGAGTGGCCTTCTCAGTCGAACCGTTCACGTTGCGAGCTGCTG | | | |
| | | GCGGCCGCAACATTTTGGCGCCAGAACTGGGACCTGAAGAATGGCA | | | |
| | | GAGAGATGCTAAGAGGAACGCTGCTTTGGAGCTCCACAGGAAAGGA | | | |
| | | TCTTCGTATCGGACATCGGAGCAACGGACAGGTACACATGCTAGCGC | | | |
| | | TAGCTTAAAATTTCAGTTTTGTAAAGTGTTGCTGAGGATGTGGTAGG | | | |
| | | ATACGAATTAAGCTTGAATCAGTGCTAACCCAACGCTGGTTCTGCTTG | | | |
| | | GGTCAGCAGCGTGTTAATCGGAACTAGAAACGGAAACAGGCAGGTT | | | |
| | | AGCCGCAGCTTTTTAGGAAGCTGCTTAGGTGGAAGAAGAAAGGGTTT | | | |
| | | AAAGTCATGGATCAGGCGGTTGCCCATAGTTTTCAGGAGTTGTTTCA | | | |
| | | GGCCAGAGGAGTAAGGCTTGAAGTACAATTAGTAAAAAATTTTTTAG | | | |
| | | GTAAGATAGATAGCTGTTGCCCATGGTTCAAGGAAGAAGAAACACTA | | | |
| | | GATTGTGGAACCTGGGAGAAAGTTGGTGAGGCCTTAAAAATCACTCA | | | |
| | | GGCAGATAATTTTACCCTAGGCCTCTGGGCACTCATAAATGATGCAAT | | | |
| | | AAAAGATGCCACTTCCCCAGGGCTAAGTTGCCCCCAGGCGGAGCTTG | | | |
| | | TGGTATCTCAGGAGGAGTGCCTGTCAGAGAGGGCCTCCTCAGAAAAA | | | |
| | | GATCTTCTTAACTCAAAAATTGATAAATGTGGAAACTCGGATGAAAA | | | |
| | | ACTGATTTTTAACAAAAATCACTCAGATAGAGGAGCTGCCCATTACCT | | | |
| | | TAATGAGAATTGGTCCTCTTGTGAATCTCCTGCTCAACCTGTAGTCCC | | | |
| | | CACTTCGGGAGGTGCCACTCATAGGGACACACGACTAAGCGAGTTAG | | | |
| | | AGTTTGAGATTAAGCTTCAGAGGCTGACTAATGAGCTTCGGGAACTA | | | |
| | | AAAAAGATGTCAGAAGCGGAGAAGAGTAACTCTTCTGTAGTTCACCA | | | |
| | | GGTGCCGCTAGAAAAGGTTGTGAGTCAGGCTCATGGGAAAGGACAG | | | |
| | | AATATCTCTAATACGCTAGCCTTTCCTGTGGTTGAGGTAGTTGATCAG | | | |
| | | CAAGATACTAGGGGCAGACATTACCAGACCTTAGATTTCAAGTTGAT | | | |
| | | AAAAGAGTTAAAGGCGGCTGTTGTGCAATATGGCCCTTCAGCCCCAT | | | |
| | | TCACTCAAGCATTACTGGACACAGTTGTGGAGTCACACTTAACCCCTT | | | |
| | | TAGATTGGAAGACTCTTTCTAAGGCTACCCTGTCAGGAGGAGATTTTT | | | |
| | | TGCTTTGGGATTCTGAATGGCGAGACGCCAGTAAGAAAACTGCTGCT | | | |
| | | TCTAACGCTCAGGCTGGTAATTCAGACTGGGATAGCAACATGCTTTTA | | | |
| | | GGAGAGGGCCCTTATGAGGGACAGACAAATCAGATTGATTTTCCCGT | | | |
| | | TGCAGTGTACGCGCAAATTGCGACGGCCGCACGCCGTGCTTGGGGA | | | |
| | | AGGTTGCCAGTCAAAGGAGAGATTGGTGGAAGTTTAGCTAGCATTCG | | | |
| | | GCAGAGTTCTGATGAACCATATCAGGATTTTGTGGACAGGCTATTGA | | | |
| | | TTTCAGCTAGTAGAATCCTTGGAAATCCGGACACGGGAAGTCCTTTCG | | | |
| | | TTATGCAATTGGCTTATGAGAATGCTAACGCAATTTGCCGAGCTGCGA | | | |
| | | TTCAACCGCATAAGGGAACGACAGATTTGGCGGGATATGTCCGTCTT | | | |
| | | TGCGCAGACATCGGGCCTTCCTGCGAGACCTTGCAGGGAACCCACGC | | | |
| | | GCAGGCAATGTTCTCTAGGAAACGAGGGAATAGTGCATGCTTTAAAT | | | |
| | | GTGGAAGTTTAGATCATTTTAGAATTGATTGTCCTCAGAACAAGGGC | | | |
| | | GCCGAGGTTAGACAAACAGGCCGTGCCCCGGGAATATGTCCCCGATG | | | |
| | | TGGAAAGGGCCGCCACTGGGCGAAAGATTGCAAGCATAAAACGAGG | | | |
| | | GTTTTGAGCCGCCCGGTGCCGGGAAACGAGGAAAGGGGTCAGCCCC | | | |
| | | AGGCCCCAAGTTACTCAAAGAAGACAGCTTATGGGGCTCTAAATCTG | | | |
| | | CTGCCCAGCCAACAAGATCAGTTCTTGAGCTTGTCAGGTCAAACCCAG | | | |
| | | GAAACGCAAGACTGGACCTCTGTTCCACTGTCCATGCAGCATTAACCC | | | |
| | | CAGAAGTGGGAGTCCAAACTCTGCCTACCGGAGTCTTTGGACCACTA | | | |
| | | CCTGTAGGAACCTGTGGTTTTCTCTTAGGACGAAGCAGTTCTATTGTA | | | |
| | | GAAGGCCTGCAGATTTATCCAGGTGTTATAAGTAATGATTATGAGGG | | | |
| | | AGAAATTAAAATCATAGCCGCTTGCCCTCGTGGTGCTATAACTATACC | | | |
| | | CGCTAATCAGAAAATTGCTCAACTTACCTTGATCCCCTTGCGCTGGTC | | | |
| | | ACTATCTAAATTCTCTAAAAATGAAGAAGGACAGATTAACTTTGACTC | | | |
| | | CTCTGGCGTAAATTGGGTGAAATCTATCACTAATCAGAGACCTAACCT | | | |
| | | TAAATTGATTCTTGATGGAAAAAGCTTTGAAGGATTAATAGATACCG | | | |
| | | GGGCCGATGTAACCATTATTAGAGGGCAGGACTGGCCCTCAAACTGG | | | |
| | | CCCCTGTCTGTTTCCTTGACTCACCTTCAAGGAATTGGTTATGCCAGTA | | | |
| | | ACCCAAAACGTAGTTCCAAATTGCTAACCTGGAGAGATGAGGATGGA | | | |
| | | AAATCAGGAAATATTCAGCCGTATGTTATGCAAAATTTGCCTGTAACC | | | |
| | | CTGTGGGGAAGAGATCTGTTGTCACAGATGGGCGTTATCCTGTGCAG | | | |
| | | TTCTAAGGAAATGGTGACTGAACAGACGTTCAGGCAGGGACCCCTGC | | | |
| | | CTGATCGTGGACTAATAAAGAAGGGACAGAAAATTAAGACTTTTGAA | | | |
| | | GATCTTAAACCCCACTCTAACGTGAGAGGTTTAAAGTATTTTCAGTAG | | | |
| | | TGGCCGCTGTCTTGCCTGCATCCCACGCCGAAAAAATTCAATGGCGTA | | | |
| | | ATGATATTCCGGTGTGGGTAGATCAGTGGTCTTTACCTAAAGAGAAA | | | |
| | | ATAGAGGCCGCTTCTCTGCTAGTGCAGGAGCAGTTAGAAGCAGGACA | | | |
| | | TTTGGTGGAGTCTCATTCTCCCTGGAATACACCCATTTTCATTATCAGG | | | |
| | | AAGAAATCGGGAAAATGGAGACTGTTGCAAGATTTAAGAAAGGTTA | | | |
| | | ATGAAACCATGGTACTTATGGGAACTTTACAACCGGGGCTCCCCTCCC | | | |
| | | CAGTAGCCATTCCTAAGGGATACTATAAGATTGTTATAGATTTGAAAG | | | |
| | | ATTGTTTCTTTACCATCCCTTTGCATCCAGAGGATTGTGAGAGATTTGC | | | |
| | | TTTTAGTGTTCCTTCTGTAAATTTCAAGGAACCCATGAAAAGATATCA | | | |
| | | ATGGACAGTTCTCCCGCAGGGGATGGCTAATAGTCCCACCTTATGTCA | | | |
| | | AAAGTTTGTGGCAAAGGCAATTCAGCCTGTTAGACAACAATGGCCAA | | | |
| | | ATATTTACATCATTCATTTCACAGATGATGTTTTGATGGCGGGAAAGG | | | |
| | | ACCCCCAAGATTTGCTTTTGTGTTATGGAGACTTACGAAAGGCCCTGG | | | |
| | | CTGATAAGGGATTACAAATTGCTTCTGAAAAGATACAAACTCAGGAT | | | |
| | | CCTTATAATTATTTGGGTTTTAGACTCACTGACCAAGCTGTTTTTCACC | | | |
| | | AGAAAATTGTTATTCGTAGAGATAACTTAAGGACCTTAAATGATTTTC | | | |
| | | AAAAATTGTTAGGTGATATAAACTGGCTTCGCCCCTATCTAAAGCTTA | | | |
| | | CTACAGGGGAGTTGAAACCTTTATTTGATATTCTTAAAGGGAGTTCTG | | | |
| | | ATCCTACTTCCCCTAGATCCCTAACCTCAGAAGGTTTACTGGCCTTACA | | | |
| | | GCTAGTGGAAAAGGCTATTGAAGAACAGTTTGTCACTTACATAGATT | | | |
| | | ACTCCCTGCCGCTGCACCTGTTAATTTTTAACACGACTCATGTGCCTAC | | | |
| | | GGGATTGCTATGGCAAAAATTTCCTATAATGTGGATACATTCAAGGAT | | | |
| | | TTCTCCCAAACGTAATATTTTGCCATATCATGAAGCAGTGGCTCAGAT | | | |
| | | GATTATCACTGGAAGAAGGCAGGCATTGACTTATTTTGGAAAGGAGC | | | |
| | | CAGATATCATTGTCCAGCCTTACAGCGTGAGTCAGGACACTTGGCTG | | | |
| | | AAACAGCATAGTACAGATTGGTTGCTTGCACAATTAGGGTTTGAAGG | | | |
| | | AACTATAGATAGCCACTACCCCCAAGATAGGTTGATAAAATTCTTAAA | | | |
| | | TGTACATGATATGATATTTCCTAAGATGACTTCCTTACAGCCTTTAAAT | | | |
| | | AATGCTCTATTGATTTTTACTGATGGCTCCTCTAAAGGGCGAGCTGGA | | | |
| | | TATCTTATTAGTAATCAACAGGTTATCGTAGAGACTCCTGGTCTCTCG | | | |
| | | GCTCAGCTCGCCGAACTAACAGCAGTACTGAAGGTTTTTCAGTCTGTA | | | |
| | | CAGGAGGCTTTTAATATTTTTACTGACAGTTTATATGTTGCTCAGTCA | | | |
| | | GTACCCTTATTGGAAACCTGTGGTACTTTTAACTTCAATACGCCGTCA | | | |
| | | GGATCTTTATTTTCAGAATTACAAAACATCATTCTCGCCCGGAAAAAT | | | |
| | | CCGTTTTATATTGGCCACATACGGTCTCACTCTGGTCTTCCTGGACCTC | | | |
| | | TGGCAGAGGGTAATAATTGCATTGACAGAGCTCTAATAGGAGAAGCC | | | |
| | | TTAGTTTCAGATCGGGTTGCTTTGGCCCAACGTGATCATGAAAGGTTT | | | |
| | | CATCTCTCTAGCCATACCCTAAGGCTCCGACATAAGATCACCAAGGAG | | | |
| | | CAAGCGAGAATGATTGTAAAACAATGTCCTAAATGTATTACTTTATCT | | | |
| | | CCAGTGCCGCATCTAGGAGTTAATCCTAGAGGCCTTATGCCTAATCAT | | | |
| | | ATTTGGCAAATGGATATAACCCATTATGCAGAATTTGGAAAACTAAA | | | |
| | | ATATATACATGTTTGCATTGATACTTGTTCAGGATTTCTCTTTGCTTCTC | | | |
| | | TGCATACAGGAGAAGCTTCAAAAAACGTAATTGATCATTGCCTACAA | | | |
| | | GCATTTAATGCCATGGGATTACCTAAACTTATTAAGACAGACAATGG | | | |
| | | GCCATCTTATTCCAGTAAAAACTTTATTTCATTCTGTAAAGAATTCGGT | | | |
| | | ATTAAACATAAAACTGGAATTCCTTACAACCCCATGGGACAAGGAAT | | | |
| | | AGTTGAACGTGCTCATCGCACCTTAAAGAATTGGCTCTTTAAGACAAA | | | |
| | | AGAGGGGCAGCTATATCCCCCAAGGTCTCCAAAGGCCCACCTTGCCT | | | |
| | | TCACCTTATTTGTCCTAAATTTCTTGCACACCGATATCAAGGGCCAGTC | | | |
| | | TGCAGCGGATCGCCACTGGCATCCAGTTACTTCTAATTCTTATGCATT | | | |
| | | GGTAAAATGGAAGGACCCCCTGACTAATGAATGGAAGGGTCCAGAT | | | |
| | | CCAGTTCTAATTTGGGGTAGAGGCTCAGTTTGTGTTTTTTCACGAGAT | | | |
| | | GAAGATGGAGCACGGTGGCTGCCAGAGAGATTAATTCGTCAGACGA | | | |
| | | ACACAGATTCTGACTCTTCTGGTAAGTATCATTCTAAAGACTAAAATT | | | |
| | | CCTTTTGTGCTTAAAATTCAGCTGAGAGCAACAGCTCTCAAAGCTGTT | | | |
| | | CTCCAGCTACTCTCTGAGCCAGCTCCCGACAGGAGGCCGGAGACTAG | | | |
| | | CCTCAGCTTTACAATTTGCATTTAAATAAAGTACCTAGACTTCCCCGAA | | | |
| | | AAAAGTTCTGCTTTTCTACTTTCTCACTGTCTTTCAAGATTTTGTCTTTC | | | |
| | | AAGCAGGTAAATCAACATTCTCGAGGCGGACCAGCGGATGTGCATCC | | | |
| | | CCGCCCCCCTAGAGCACTCAGGTGGCAGCTGTTATCCCCAGTCTCAGG | | | |
| | | ACATTCCAGCATGTGGCCTTCAGTCTGAGTTAAAAATTAGGTTTACCC | | | |
| | | AGAGGACTAGAATAGTAGATATTTCTATATTAATAAAGATTGGTTTTT | | | |
| | | ATTTTGATAGACAGGCTTAGCCCCTTAGCTGACCTCTGGCTTTTCACCC | | | |
| | | TTGCTGTTACTGCAAGGTGTCTTTAGCTCAATAAGGCTGTGGAAAAAA | | | |
| | | ACAGGGATGAGGAGGAACGGCTCCCAGCTCCTATTTTAGCCACAAAT | | | |
| | | CGTGGTGTTACTAACGACATAATTCTTGCTTAGGCTTTGCTAAATCTG | | | |
| | | AGGTTGATAATTCTCCTTTAGGAGCTGCACAGCGCTCAGAACTGTGCA | | | |
| | | TACTGATTTGTGATGGTACAAATTCAGTATGGGCATCGCTTGGTGCA | | | |
| | | GATGGAGGTACTGCAAGGAAAGGTCCCAGCTTGACCATTTCTGAGTT | | | |
| | | TCCTGTGAGATAAACCCGGTTTGAAAGAGGTTGGTACCAAATTATAT | | | |
| | | ATCCCTCGGCTCTACCTCGCCTCCCCAAAAGGTACCAGAGCCACAGGT | | | |
| | | GTGGATTTTAACAGAATCCACGGGAGGAATCGGGTCCATGTCCACCC | | | |
| | | AAGCCAAGGTTAAAAGCCCACTCATCTACGGATGAGAAAATCATTTG | | | |
| | | ATCACCTCAGTTAAGCGCTGCCTTATTTTAACTTAATTAATAGGGGGG | | | |
| | | AGAGAGATTGGAGACTTACTATTGAAAGGGCAAGCCCTTCACTGCCT | | | |
| | | CCCACCCAAATAAAAAAGCCAATTGGCCTTGTACTACAGAGCTGGCC | | | |
| | | GGACCCCTTATCCCTGTTACCCACCAATCATCCAAAAATGCGGAGGAA | | | |
| | | TATCAACTTAGTGTTATTCTTATTATAGTGTATTTCACACTTGTTCAGTC | | | |
| | | AAACTTAGCCAGAGTTCCAACGCCCTACTTAAAATTCAACTAGAAAGT | | | |
| | | TACCTACCAAGTACTAATTAGCATTATAAAGTCAGAGCCTACAGCTCC | | | |
| | | AGGCTTTTCAGTTAGTTGTTTACTAAGATAAGAAAAGACAGTCTTAGC | | | |
| | | CAGATACAGTTTACCATAATAAAAGTTAAAGAATCCCAGGGAAGCAA | | | |
| | | GTTTTTTCTTTTAGCCCTAGATTCCAGGCAGAACTATTGAGCATAGAT | | | |
| | | AATTTTTCCCCCTCAGGCCAGCTTTTTCTTTTTTTTTAATTTTGTTAATA | | | |
| | | AAAGGGAGGAGATGTAGTCTCCCCTCCCCCAGCCTGAAACCTGCTTG | | | |
| | | CTCAGGGGTGGAGCTTCCCGCTCATCGCTCTGCCACGCCCACTGCTG | | | |
| | | GAACCTGCGGAGCCACACACGTGCACCTTTCTACTGGACCAGAGATT | | | |
| | | ATTCGGCGGGAATCGGGTCCCCTCCCCCTTCCTTCATAACTAGTGTCC | | | |
| | | CAACAATAAAATTTGAGCTTTGATCAGAATGAATTTGTCTTGGCTCCG | | | |
| | | TTTCTTCTTTCGCCCCGTCTAGATTCCTCTCTTACAGCTCGAGTGGCCT | | | |
| | | TCTCAGTCGAACCGTTCACGTTGCGAGCTGCTGGCGGCCGCAACAGG | | | |
| | | GGATCCAGACATGATAAGATACATTGATGAGTTTGGACAAACCACAA | | | |
| | | CTAGAATGCAGTGAAAAAAATGCTTTATTTGTGAAATTTGTGATGCTA | | | |
| | | TTGCTTTATTTGTAACCATTATAAGCTGCAATAAACAAGTTAACAACA | | | |
| | | ACAATTGCATTCATTTTATGTTTCAGGTTCAGGGGGAGGTGTGGGAG | | | |
| | | GTTTTTTCGGATCCTCTAGAGTCGACCTGCAGGCATGCAAGCTTGGCG | | | |
| | | TAATCATGGTCATAGCTGTTTCCTGTGTGAAATTGTTATCCGCTCACAA | | | |
| | | TTCCACACAACATACGAGCCGGAAGCATAAAGTGTAAAGCCTGGGGT | | | |
| | | GCCTAATGAGTGAGCTAACTCACATTAATTGCGTTGCGCTCACTGCCC | | | |
| | | GCTTTCCAGTCGGGAAACCTGTCGTGCCAGCTGCATTAATGAATCGG | | | |
| | | CCAACGCGCGGGGAGAGGCGGTTTGCGTATTGGGCGCTCTTCCGCTT | | | |
| | | CCTCGCTCACTGACTCGCTGCGCTCGGTCGTTCGGCTGCGGCGAGCG | | | |
| | | GTATCAGCTCACTCAAAGGCGGTAATACGGTTATCCACAGAATCAGG | | | |
| | | GGATAACGCAGGAAAGAACATGTGAGCAAAAGGCCAGCAAAAGGCC | | | |
| | | AGGAACCGTAAAAAGGCCGCGTTGCTGGCGTTTTTCCATAGGCTCCG | | | |
| | | CCCCCCTGACGAGCATCACAAAAATCGACGCTCAAGTCAGAGGTGGC | | | |
| | | GAAACCCGACAGGACTATAAAGATACCAGGCGTTTCCCCCTGGAAGC | | | |
| | | TCCCTCGTGCGCTCTCCTGTTCCGACCCTGCCGCTTACCGGATACCTGT | | | |
| | | CCGCCTTTCTCCCTTCGGGAAGCGTGGCGCTTTCTCATAGCTCACGCT | | | |
| | | GTAGGTATCTCAGTTCGGTGTAGGTCGTTCGCTCCAAGCTGGGCTGT | | | |
| | | GTGCACGAACCCCCCGTTCAGCCCGACCGCTGCGCCTTATCCGGTAAC | | | |
| | | TATCGTCTTGAGTCCAACCCGGTAAGACACGACTTATCGCCACTGGCA | | | |
| | | GCAGCCACTGGTAACAGGATTAGCAGAGCGAGGTATGTAGGCGGTG | | | |
| | | CTACAGAGTTCTTGAAGTGGTGGCCTAACTACGGCTACACTAGAAGG | | | |
| | | ACAGTATTTGGTATCTGCGCTCTGCTGAAGCCAGTTACCTTCGGAAAA | | | |
| | | AGAGTTGGTAGCTCTTGATCCGGCAAACAAACCACCGCTGGTAGCGG | | | |
| | | TGGTTTTTTTGTTTGCAAGCAGCAGATTACGCGCAGAAAAAAAGGAT | | | |
| | | CTCAAGAAGATCCTTTGATCTTTTCTACGGGGTCTGACGCTCAGTGGA | | | |
| | | ACGAAAACTCACGTTAAGGGATTTTGGTCATGAGATTATCAAAAAGG | | | |
| | | ATCTTCACCTAGATCCTTTTAAATTAAAAATGAAGTTTTAAATCAATCT | | | |
| | | AAAGTATATATGAGTAAACTTGGTCTGACAGTTACCAATGCTTAATCA | | | |
| | | GTGAGGCACCTATCTCAGCGATCTGTCTATTTCGTTCATCCATAGTTG | | | |
| | | CCTGACTCCCCGTCGTGTAGATAACTACGATACGGGAGGGCTTACCA | | | |
| | | TCTGGCCCCAGTGCTGCAATGATACCGCGAGACCCACGCTCACCGGC | | | |
| | | TCCAGATTTATCAGCAATAAACCAGCCAGCCGGAAGGGCCGAGCGCA | | | |
| | | GAAGTGGTCCTGCAACTTTATCCGCCTCCATCCAGTCTATTAATTGTTG | | | |
| | | CCGGGAAGCTAGAGTAAGTAGTTCGCCAGTTAATAGTTTGCGCAACG | | | |
| | | TTGTTGCCATTGCTACAGGCATCGTGGTGTCACGCTCGTCGTTTGGTA | | | |
| | | TGGCTTCATTCAGCTCCGGTTCCCAACGATCAAGGCGAGTTACATGAT | | | |
| | | CCCCCATGTTGTGCAAAAAAGCGGTTAGCTCCTTCGGTCCTCCGATCG | | | |
| | | TTGTCAGAAGTAAGTTGGCCGCAGTGTTATCACTCATGGTTATGGCA | | | |
| | | GCACTGCATAATTCTCTTACTGTCATGCCATCCGTAAGATGCTTTTCTG | | | |
| | | TGACTGGTGAGTACTCAACCAAGTCATTCTGAGAATAGTGTATGCGG | | | |
| | | CGACCGAGTTGCTCTTGCCCGGCGTCAATACGGGATAATACCGCGCC | | | |
| | | ACATAGCAGAACTTTAAAAGTGCTCATCATTGGAAAACGTTCTTCGGG | | | |
| | | GCGAAAACTCTCAAGGATCTTACCGCTGTTGAGATCCAGTTCGATGTA | | | |
| | | ACCCACTCGTGCACCCAACTGATCTTCAGCATCTTTTACTTTCACCAGC | | | |
| | | GTTTCTGGGTGAGCAAAAACAGGAAGGCAAAATGCCGCAAAAAAGG | | | |
| | | GAATAAGGGCGACACGGAAATGTTGAATACTCATACTCTTCCTTTTTC | | | |
| | | AATATTATTGAAGCATTTATCAGGGTTATTGTCTCATGAGCGGATACA | | | |
| | | TATTTGAATGTATTTAGAAAAATAAACAAATAGGGGTTCCGCGCACAT | | | |
| | | TTCCCCGAAAAGTGCCACCTGACGTCTAAGAAACCATTATTATCATGA | | | |
| | | CATTAACCTATAAAAATAGGCGTATCACGAGGCCCTTTCGTCTCGCGC | | | |
| | | GTTTCGGTGATGACGGTGAAAACCTCTGACACATGCAGCTCCCGGAG | | | |
| | | ACGGTCACAGCTTGTCTGTAAGCGGATGCCGGGAGCAGACAAGCCC | | | |
| | | GTCAGGGCGCGTCAGCGGGTGTTGGCGGGTGTCGGGGCTGGCTTAA | | | |
| | | CTATGCGGCATCAGAGCAGATTGTACTGAGAGTGCACCATATGCGGT | | | |
| | | GTGAAATACCGCACAGATGCGTAAGGAGAAAATACCGCATCAGGCG | | | |
| | | CCATTCGCCATTCAGGCTGCGCAACTGTTGGGAAGGGCGATCGGTGC | | | |
| | | GGGCCTCTTCGCTATTACGCCAGCTGGCGAAAGGGGGATGTGCTGCA | | | |
| | | AGGCGATTAAGTTGGGTAACGCCAGGGTTTTCCCAGTCACGACGTTG | | | |
| | | TAAAACGACGGCCAGT | | | |
| DRIVER_ | PLV10047 | GAATTCGAGCTTGCATGCCTGCAGGTCGTTACATAACTTACGGTAAAT | 315 | N/A | |
| pCMV_R- | | GGCCCGCCTGGCTGACCGCCCAACGACCCCCGCCCATTGACGTCAAT | | | |
| U5- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| LTR_v2 | | AAGTGTATCATATGCCAAGTACGCCCCCTATTGACGTCAATGACGGTA | | | |
| | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGCTTTGATCAGAATGAATTT | | | |
| | | GTCTTGGCTCCGTTTCTTCTTTCGCCCCGTCTAGATTCCTCTCTTACAGC | | | |
| | | TCGAGTGGCCTTCTCAGTCGAACCGTTCACGTTGCGAGCTGCTGGCG | | | |
| | | GCCGCAACATTTTGGCGCCAGAACTGGGACCTGAAGAATGGCAGAG | | | |
| | | AGATGCTAAGAGGAACGCTGCTTTGGAGCTCCACAGGAAAGGATCTT | | | |
| | | CGTATCGGACATCGGAGCAACGGACAGGTACACATGCTAGCGCTAGC | | | |
| | | TTAAAATTTCAGTTTTGTAAAGTGTTGCTGAGGATGTGGTAGGATAC | | | |
| | | GAATTAAGCTTGAATCAGTGCTAACCCAACGCTGGTTCTGCTTGGGTC | | | |
| | | AGCAGCGTGTTAATCGGAACTAGAAACGGAAACAGGCAGGTTAGCC | | | |
| | | GCAGCTTTTTAGGAAGCTGCTTAGGTGGAAGAAGAAAGGGTTTAAA | | | |
| | | GTCATGGATCAGGCGGTTGCCCATAGTTTTCAGGAGTTGTTTCAGGC | | | |
| | | CAGAGGAGTAAGGCTTGAAGTACAATTAGTAAAAAATTTTTTAGGTA | | | |
| | | AGATAGATAGCTGTTGCCCATGGTTCAAGGAAGAAGAAACACTAGAT | | | |
| | | TGTGGAACCTGGGAGAAAGTTGGTGAGGCCTTAAAAATCACTCAGGC | | | |
| | | AGATAATTTTACCCTAGGCCTCTGGGCACTCATAAATGATGCAATAAA | | | |
| | | AGATGCCACTTCCCCAGGGCTAAGTTGCCCCCAGGCGGAGCTTGTGG | | | |
| | | TATCTCAGGAGGAGTGCCTGTCAGAGAGGGCCTCCTCAGAAAAAGAT | | | |
| | | CTTCTTAACTCAAAAATTGATAAATGTGGAAACTCGGATGAAAAACTG | | | |
| | | ATTTTTAACAAAAATCACTCAGATAGAGGAGCTGCCCATTACCTTAAT | | | |
| | | GAGAATTGGTCCTCTTGTGAATCTCCTGCTCAACCTGTAGTCCCCACTT | | | |
| | | CGGGAGGTGCCACTCATAGGGACACACGACTAAGCGAGTTAGAGTTT | | | |
| | | GAGATTAAGCTTCAGAGGCTGACTAATGAGCTTCGGGAACTAAAAAA | | | |
| | | GATGTCAGAAGCGGAGAAGAGTAACTCTTCTGTAGTTCACCAGGTGC | | | |
| | | CGCTAGAAAAGGTTGTGAGTCAGGCTCATGGGAAAGGACAGAATAT | | | |
| | | CTCTAATACGCTAGCCTTTCCTGTGGTTGAGGTAGTTGATCAGCAAGA | | | |
| | | TACTAGGGGCAGACATTACCAGACCTTAGATTTCAAGTTGATAAAAG | | | |
| | | AGTTAAAGGCGGCTGTTGTGCAATATGGCCCTTCAGCCCCATTCACTC | | | |
| | | AAGCATTACTGGACACAGTTGTGGAGTCACACTTAACCCCTTTAGATT | | | |
| | | GGAAGACTCTTTCTAAGGCTACCCTGTCAGGAGGAGATTTTTTGCTTT | | | |
| | | GGGATTCTGAATGGCGAGACGCCAGTAAGAAAACTGCTGCTTCTAAC | | | |
| | | GCTCAGGCTGGTAATTCAGACTGGGATAGCAACATGCTTTTAGGAGA | | | |
| | | GGGCCCTTATGAGGGACAGACAAATCAGATTGATTTTCCCGTTGCAG | | | |
| | | TGTACGCGCAAATTGCGACGGCCGCACGCCGTGCTTGGGGAAGGTT | | | |
| | | GCCAGTCAAAGGAGAGATTGGTGGAAGTTTAGCTAGCATTCGGCAG | | | |
| | | AGTTCTGATGAACCATATCAGGATTTTGTGGACAGGCTATTGATTTCA | | | |
| | | GCTAGTAGAATCCTTGGAAATCCGGACACGGGAAGTCCTTTCGTTAT | | | |
| | | GCAATTGGCTTATGAGAATGCTAACGCAATTTGCCGAGCTGCGATTC | | | |
| | | AACCGCATAAGGGAACGACAGATTTGGGGGATATGTCCGTCTTTGC | | | |
| | | GCAGACATCGGGCCTTCCTGCGAGACCTTGCAGGGAACCCACGCGCA | | | |
| | | GGCAATGTTCTCTAGGAAACGAGGGAATAGTGCATGCTTTAAATGTG | | | |
| | | GAAGTTTAGATCATTTTAGAATTGATTGTCCTCAGAACAAGGGCGCC | | | |
| | | GAGGTTAGACAAACAGGCCGTGCCCCGGGAATATGTCCCCGATGTG | | | |
| | | GAAAGGGCCGCCACTGGGCGAAAGATTGCAAGCATAAAACGAGGGT | | | |
| | | TTTGAGCCGCCCGGTGCCGGGAAACGAGGAAAGGGGTCAGCCCCAG | | | |
| | | GCCCCAAGTTACTCAAAGAAGACAGCTTATGGGGCTCTAAATCTGCT | | | |
| | | GCCCAGCCAACAAGATCAGTTCTTGAGCTTGTCAGGTCAAACCCAGG | | | |
| | | AAACGCAAGACTGGACCTCTGTTCCACTGTCCATGCAGCATTAACCCC | | | |
| | | AGAAGTGGGAGTCCAAACTCTGCCTACCGGAGTCTTTGGACCACTAC | | | |
| | | CTGTAGGAACCTGTGGTTTTCTCTTAGGACGAAGCAGTTCTATTGTAG | | | |
| | | AAGGCCTGCAGATTTATCCAGGTGTTATAAGTAATGATTATGAGGGA | | | |
| | | GAAATTAAAATCATAGCCGCTTGCCCTCGTGGTGCTATAACTATACCC | | | |
| | | GCTAATCAGAAAATTGCTCAACTTACCTTGATCCCCTTGCGCTGGTCA | | | |
| | | CTATCTAAATTCTCTAAAAATGAAGAAGGACAGATTAACTTTGACTCC | | | |
| | | TCTGGCGTAAATTGGGTGAAATCTATCACTAATCAGAGACCTAACCTT | | | |
| | | AAATTGATTCTTGATGGAAAAAGCTTTGAAGGATTAATAGATACCGG | | | |
| | | GGCCGATGTAACCATTATTAGAGGGCAGGACTGGCCCTCAAACTGGC | | | |
| | | CCCTGTCTGTTTCCTTGACTCACCTTCAAGGAATTGGTTATGCCAGTAA | | | |
| | | CCCAAAACGTAGTTCCAAATTGCTAACCTGGAGAGATGAGGATGGAA | | | |
| | | AATCAGGAAATATTCAGCCGTATGTTATGCAAAATTTGCCTGTAACCC | | | |
| | | TGTGGGGAAGAGATCTGTTGTCACAGATGGGCGTTATCCTGTGCAGT | | | |
| | | TCTAAGGAAATGGTGACTGAACAGACGTTCAGGCAGGGACCCCTGCC | | | |
| | | TGATCGTGGACTAATAAAGAAGGGACAGAAAATTAAGACTTTTGAAG | | | |
| | | ATCTTAAACCCCACTCTAACGTGAGAGGTTTAAAGTATTTTCAGTAGT | | | |
| | | GGCCGCTGTCTTGCCTGCATCCCACGCCGAAAAAATTCAATGGCGTA | | | |
| | | ATGATATTCCGGTGTGGGTAGATCAGTGGTCTTTACCTAAAGAGAAA | | | |
| | | ATAGAGGCCGCTTCTCTGCTAGTGCAGGAGCAGTTAGAAGCAGGACA | | | |
| | | TTTGGTGGAGTCTCATTCTCCCTGGAATACACCCATTTTCATTATCAGG | | | |
| | | AAGAAATCGGGAAAATGGAGACTGTTGCAAGATTTAAGAAAGGTTA | | | |
| | | ATGAAACCATGGTACTTATGGGAACTTTACAACCGGGGCTCCCCTCCC | | | |
| | | CAGTAGCCATTCCTAAGGGATACTATAAGATTGTTATAGATTTGAAAG | | | |
| | | ATTGTTTCTTTACCATCCCTTTGCATCCAGAGGATTGTGAGAGATTTGC | | | |
| | | TTTTAGTGTTCCTTCTGTAAATTTCAAGGAACCCATGAAAAGATATCA | | | |
| | | ATGGACAGTTCTCCCGCAGGGGATGGCTAATAGTCCCACCTTATGTCA | | | |
| | | AAAGTTTGTGGCAAAGGCAATTCAGCCTGTTAGACAACAATGGCCAA | | | |
| | | ATATTTACATCATTCATTTCACAGATGATGTTTTGATGGCGGGAAAGG | | | |
| | | ACCCCCAAGATTTGCTTTTGTGTTATGGAGACTTACGAAAGGCCCTGG | | | |
| | | CTGATAAGGGATTACAAATTGCTTCTGAAAAGATACAAACTCAGGAT | | | |
| | | CCTTATAATTATTTGGGTTTTAGACTCACTGACCAAGCTGTTTTTCACC | | | |
| | | AGAAAATTGTTATTCGTAGAGATAACTTAAGGACCTTAAATGATTTTC | | | |
| | | AAAAATTGTTAGGTGATATAAACTGGCTTCGCCCCTATCTAAAGCTTA | | | |
| | | CTACAGGGGAGTTGAAACCTTTATTTGATATTCTTAAAGGGAGTTCTG | | | |
| | | ATCCTACTTCCCCTAGATCCCTAACCTCAGAAGGTTTACTGGCCTTACA | | | |
| | | GCTAGTGGAAAAGGCTATTGAAGAACAGTTTGTCACTTACATAGATT | | | |
| | | ACTCCCTGCCGCTGCACCTGTTAATTTTTAACACGACTCATGTGCCTAC | | | |
| | | GGGATTGCTATGGCAAAAATTTCCTATAATGTGGATACATTCAAGGAT | | | |
| | | TTCTCCCAAACGTAATATTTTGCCATATCATGAAGCAGTGGCTCAGAT | | | |
| | | GATTATCACTGGAAGAAGGCAGGCATTGACTTATTTTGGAAAGGAGC | | | |
| | | CAGATATCATTGTCCAGCCTTACAGCGTGAGTCAGGACACTTGGCTG | | | |
| | | AAACAGCATAGTACAGATTGGTTGCTTGCACAATTAGGGTTTGAAGG | | | |
| | | AACTATAGATAGCCACTACCCCCAAGATAGGTTGATAAAATTCTTAAA | | | |
| | | TGTACATGATATGATATTTCCTAAGATGACTTCCTTACAGCCTTTAAAT | | | |
| | | AATGCTCTATTGATTTTTACTGATGGCTCCTCTAAAGGGCGAGCTGGA | | | |
| | | TATCTTATTAGTAATCAACAGGTTATCGTAGAGACTCCTGGTCTCTCG | | | |
| | | GCTCAGCTCGCCGAACTAACAGCAGTACTGAAGGTTTTTCAGTCTGTA | | | |
| | | CAGGAGGCTTTTAATATTTTTACTGACAGTTTATATGTTGCTCAGTCA | | | |
| | | GTACCCTTATTGGAAACCTGTGGTACTTTTAACTTCAATACGCCGTCA | | | |
| | | GGATCTTTATTTTCAGAATTACAAAACATCATTCTCGCCCGGAAAAAT | | | |
| | | CCGTTTTATATTGGCCACATACGGTCTCACTCTGGTCTTCCTGGACCTC | | | |
| | | TGGCAGAGGGTAATAATTGCATTGACAGAGCTCTAATAGGAGAAGCC | | | |
| | | TTAGTTTCAGATCGGGTTGCTTTGGCCCAACGTGATCATGAAAGGTTT | | | |
| | | CATCTCTCTAGCCATACCCTAAGGCTCCGACATAAGATCACCAAGGAG | | | |
| | | CAAGCGAGAATGATTGTAAAACAATGTCCTAAATGTATTACTTTATCT | | | |
| | | CCAGTGCCGCATCTAGGAGTTAATCCTAGAGGCCTTATGCCTAATCAT | | | |
| | | ATTTGGCAAATGGATATAACCCATTATGCAGAATTTGGAAAACTAAA | | | |
| | | ATATATACATGTTTGCATTGATACTTGTTCAGGATTTCTCTTTGCTTCTC | | | |
| | | TGCATACAGGAGAAGCTTCAAAAAACGTAATTGATCATTGCCTACAA | | | |
| | | GCATTTAATGCCATGGGATTACCTAAACTTATTAAGACAGACAATGG | | | |
| | | GCCATCTTATTCCAGTAAAAACTTTATTTCATTCTGTAAAGAATTCGGT | | | |
| | | ATTAAACATAAAACTGGAATTCCTTACAACCCCATGGGACAAGGAAT | | | |
| | | AGTTGAACGTGCTCATCGCACCTTAAAGAATTGGCTCTTTAAGACAAA | | | |
| | | AGAGGGGCAGCTATATCCCCCAAGGTCTCCAAAGGCCCACCTTGCCT | | | |
| | | TCACCTTATTTGTCCTAAATTTCTTGCACACCGATATCAAGGGCCAGTC | | | |
| | | TGCAGCGGATCGCCACTGGCATCCAGTTACTTCTAATTCTTATGCATT | | | |
| | | GGTAAAATGGAAGGACCCCCTGACTAATGAATGGAAGGGTCCAGAT | | | |
| | | CCAGTTCTAATTTGGGGTAGAGGCTCAGTTTGTGTTTTTTCACGAGAT | | | |
| | | GAAGATGGAGCACGGTGGCTGCCAGAGAGATTAATTCGTCAGACGA | | | |
| | | ACACAGATTCTGACTCTTCTGGTAAGTATCATTCTAAAGACTAAAATT | | | |
| | | CCTTTTGTGCTTAAAATTCAGCTGAGAGCAACAGCTCTCAAAGCTGTT | | | |
| | | CTCCAGCTACTCTCTGAGCCAGCTCCCGACAGGAGGCCGGAGACTAG | | | |
| | | CCTCAGCTTTACAATTTGCATTTAAATAAAGTACCTAGACTTCCCCGAA | | | |
| | | AAAAGTTCTGCTTTTCTACTTTCTCACTGTCTTTCAAGATTTTGTCTTTC | | | |
| | | AAGCAGGTAAATCAACATTCTCGAGGCGGACCAGCGGATGTGCATCC | | | |
| | | CCGCCCCCCTAGAGCACTCAGGTGGCAGCTGTTATCCCCAGTCTCAGG | | | |
| | | ACATTCCAGCATGTGGCCTTCAGTCTGAGTTAAAAATTAGGTTTACCC | | | |
| | | AGAGGACTAGAATAGTAGATATTTCTATATTAATAAAGATTGGTTTTT | | | |
| | | ATTTTGATAGACAGGCTTAGCCCCTTAGCTGACCTCTGGCTTTTCACCC | | | |
| | | TTGCTGTTACTGCAAGGTGTCTTTAGCTCAATAAGGCTGTGGAAAAAA | | | |
| | | ACAGGGATGAGGAGGAACGGCTCCCAGCTCCTATTTTAGCCACAAAT | | | |
| | | CGTGGTGTTACTAACGACATAATTCTTGCTTAGGCTTTGCTAAATCTG | | | |
| | | AGGTTGATAATTCTCCTTTAGGAGCTGCACAGCGCTCAGAACTGTGCA | | | |
| | | TACTGATTTGTGATGGTACAAATTCAGTATGGGCATCGCTTGGTGCA | | | |
| | | GATGGAGGTACTGCAAGGAAAGGTCCCAGCTTGACCATTTCTGAGTT | | | |
| | | TCCTGTGAGATAAACCCGGTTTGAAAGAGGTTGGTACCAAATTATAT | | | |
| | | ATCCCTCGGCTCTACCTCGCCTCCCCAAAAGGTACCAGAGCCACAGGT | | | |
| | | GTGGATTTTAACAGAATCCACGGGAGGAATCGGGTCCATGTCCACCC | | | |
| | | AAGCCAAGGTTAAAAGCCCACTCATCTACGGATGAGAAAATCATTTG | | | |
| | | ATCACCTCAGTTAAGCGCTGCCTTATTTTAACTTAATTAATAGGGGGG | | | |
| | | AGAGAGATTGGAGACTTACTATTGAAAGGGCAAGCCCTTCACTGCCT | | | |
| | | CCCACCCAAATAAAAAAGCCAATTGGCCTTGTACTACAGAGCTGGCC | | | |
| | | GGACCCCTTATCCCTGTTACCCACCAATCATCCAAAAATGCGGAGGAA | | | |
| | | TATCAACTTAGTGTTATTCTTATTATAGTGTATTTCACACTTGTTCAGTC | | | |
| | | AAACTTAGCCAGAGTTCCAACGCCCTACTTAAAATTCAACTAGAAAGT | | | |
| | | TACCTACCAAGTACTAATTAGCATTATAAAGTCAGAGCCTACAGCTCC | | | |
| | | AGGCTTTTCAGTTAGTTGTTTACTAAGATAAGAAAAGACAGTCTTAGC | | | |
| | | CAGATACAGTTTACCATAATAAAAGTTAAAGAATCCCAGGGAAGCAA | | | |
| | | GTTTTTTCTTTTAGCCCTAGATTCCAGGCAGAACTATTGAGCATAGAT | | | |
| | | AATTTTTCCCCCTCAGGCCAGCTTTTTCTTTTTTTTTAATTTTGTTAATA | | | |
| | | AAAGGGAGGAGATGTAGTCTCCCCTCCCCCAGCCTGAAACCTGCTTG | | | |
| | | CTCAGGGGTGGAGCTTCCCGCTCATCGCTCTGCCACGCCCACTGCTG | | | |
| | | GAACCTGCGGAGCCACACACGTGCACCTTTCTACTGGACCAGAGATT | | | |
| | | ATTCGGCGGGAATCGGGTCCCCTCCCCCTTCCTTCATAACTAGTGTCC | | | |
| | | CAACAATAAAATTTGAGCTTTGATCAGAATGAATTTGTCTTGGCTCCG | | | |
| | | TTTCTTCTTTCGCCCCGTCTAGATTCCTCTCTTACAGCTCGAGTGGCCT | | | |
| | | TCTCAGTCGAACCGTTCACGTTGCGAGCTGCTGGCGGCCGCAACAGG | | | |
| | | GGATCCAGACATGATAAGATACATTGATGAGTTTGGACAAACCACAA | | | |
| | | CTAGAATGCAGTGAAAAAAATGCTTTATTTGTGAAATTTGTGATGCTA | | | |
| | | TTGCTTTATTTGTAACCATTATAAGCTGCAATAAACAAGTTAACAACA | | | |
| | | ACAATTGCATTCATTTTATGTTTCAGGTTCAGGGGGAGGTGTGGGAG | | | |
| | | GTTTTTTCGGATCCTCTAGAGTCGACCTGCAGGCATGCAAGCTTGGCG | | | |
| | | TAATCATGGTCATAGCTGTTTCCTGTGTGAAATTGTTATCCGCTCACAA | | | |
| | | TTCCACACAACATACGAGCCGGAAGCATAAAGTGTAAAGCCTGGGGT | | | |
| | | GCCTAATGAGTGAGCTAACTCACATTAATTGCGTTGCGCTCACTGCCC | | | |
| | | GCTTTCCAGTCGGGAAACCTGTCGTGCCAGCTGCATTAATGAATCGG | | | |
| | | CCAACGCGCGGGGAGAGGCGGTTTGCGTATTGGGCGCTCTTCCGCTT | | | |
| | | CCTCGCTCACTGACTCGCTGCGCTCGGTCGTTCGGCTGCGGCGAGCG | | | |
| | | GTATCAGCTCACTCAAAGGCGGTAATACGGTTATCCACAGAATCAGG | | | |
| | | GGATAACGCAGGAAAGAACATGTGAGCAAAAGGCCAGCAAAAGGCC | | | |
| | | AGGAACCGTAAAAAGGCCGCGTTGCTGGCGTTTTTCCATAGGCTCCG | | | |
| | | CCCCCCTGACGAGCATCACAAAAATCGACGCTCAAGTCAGAGGTGGC | | | |
| | | GAAACCCGACAGGACTATAAAGATACCAGGCGTTTCCCCCTGGAAGC | | | |
| | | TCCCTCGTGCGCTCTCCTGTTCCGACCCTGCCGCTTACCGGATACCTGT | | | |
| | | CCGCCTTTCTCCCTTCGGGAAGCGTGGCGCTTTCTCATAGCTCACGCT | | | |
| | | GTAGGTATCTCAGTTCGGTGTAGGTCGTTCGCTCCAAGCTGGGCTGT | | | |
| | | GTGCACGAACCCCCCGTTCAGCCCGACCGCTGCGCCTTATCCGGTAAC | | | |
| | | TATCGTCTTGAGTCCAACCCGGTAAGACACGACTTATCGCCACTGGCA | | | |
| | | GCAGCCACTGGTAACAGGATTAGCAGAGCGAGGTATGTAGGCGGTG | | | |
| | | CTACAGAGTTCTTGAAGTGGTGGCCTAACTACGGCTACACTAGAAGG | | | |
| | | ACAGTATTTGGTATCTGCGCTCTGCTGAAGCCAGTTACCTTCGGAAAA | | | |
| | | AGAGTTGGTAGCTCTTGATCCGGCAAACAAACCACCGCTGGTAGCGG | | | |
| | | TGGTTTTTTTGTTTGCAAGCAGCAGATTACGCGCAGAAAAAAAGGAT | | | |
| | | CTCAAGAAGATCCTTTGATCTTTTCTACGGGGTCTGACGCTCAGTGGA | | | |
| | | ACGAAAACTCACGTTAAGGGATTTTGGTCATGAGATTATCAAAAAGG | | | |
| | | ATCTTCACCTAGATCCTTTTAAATTAAAAATGAAGTTTTAAATCAATCT | | | |
| | | AAAGTATATATGAGTAAACTTGGTCTGACAGTTACCAATGCTTAATCA | | | |
| | | GTGAGGCACCTATCTCAGCGATCTGTCTATTTCGTTCATCCATAGTTG | | | |
| | | CCTGACTCCCCGTCGTGTAGATAACTACGATACGGGAGGGCTTACCA | | | |
| | | TCTGGCCCCAGTGCTGCAATGATACCGCGAGACCCACGCTCACCGGC | | | |
| | | TCCAGATTTATCAGCAATAAACCAGCCAGCCGGAAGGGCCGAGCGCA | | | |
| | | GAAGTGGTCCTGCAACTTTATCCGCCTCCATCCAGTCTATTAATTGTTG | | | |
| | | CCGGGAAGCTAGAGTAAGTAGTTCGCCAGTTAATAGTTTGCGCAACG | | | |
| | | TTGTTGCCATTGCTACAGGCATCGTGGTGTCACGCTCGTCGTTTGGTA | | | |
| | | TGGCTTCATTCAGCTCCGGTTCCCAACGATCAAGGCGAGTTACATGAT | | | |
| | | CCCCCATGTTGTGCAAAAAAGCGGTTAGCTCCTTCGGTCCTCCGATCG | | | |
| | | TTGTCAGAAGTAAGTTGGCCGCAGTGTTATCACTCATGGTTATGGCA | | | |
| | | GCACTGCATAATTCTCTTACTGTCATGCCATCCGTAAGATGCTTTTCTG | | | |
| | | TGACTGGTGAGTACTCAACCAAGTCATTCTGAGAATAGTGTATGCGG | | | |
| | | CGACCGAGTTGCTCTTGCCCGGCGTCAATACGGGATAATACCGCGCC | | | |
| | | ACATAGCAGAACTTTAAAAGTGCTCATCATTGGAAAACGTTCTTCGGG | | | |
| | | GCGAAAACTCTCAAGGATCTTACCGCTGTTGAGATCCAGTTCGATGTA | | | |
| | | ACCCACTCGTGCACCCAACTGATCTTCAGCATCTTTTACTTTCACCAGC | | | |
| | | GTTTCTGGGTGAGCAAAAACAGGAAGGCAAAATGCCGCAAAAAAGG | | | |
| | | GAATAAGGGCGACACGGAAATGTTGAATACTCATACTCTTCCTTTTTC | | | |
| | | AATATTATTGAAGCATTTATCAGGGTTATTGTCTCATGAGCGGATACA | | | |
| | | TATTTGAATGTATTTAGAAAAATAAACAAATAGGGGTTCCGCGCACAT | | | |
| | | TTCCCCGAAAAGTGCCACCTGACGTCTAAGAAACCATTATTATCATGA | | | |
| | | CATTAACCTATAAAAATAGGCGTATCACGAGGCCCTTTCGTCTCGCGC | | | |
| | | GTTTCGGTGATGACGGTGAAAACCTCTGACACATGCAGCTCCCGGAG | | | |
| | | ACGGTCACAGCTTGTCTGTAAGCGGATGCCGGGAGCAGACAAGCCC | | | |
| | | GTCAGGGCGCGTCAGCGGGTGTTGGCGGGTGTCGGGGCTGGCTTAA | | | |
| | | CTATGCGGCATCAGAGCAGATTGTACTGAGAGTGCACCATATGCGGT | | | |
| | | GTGAAATACCGCACAGATGCGTAAGGAGAAAATACCGCATCAGGCG | | | |
| | | CCATTCGCCATTCAGGCTGCGCAACTGTTGGGAAGGGCGATCGGTGC | | | |
| | | GGGCCTCTTCGCTATTACGCCAGCTGGCGAAAGGGGGATGTGCTGCA | | | |
| | | AGGCGATTAAGTTGGGTAACGCCAGGGTTTTCCCAGTCACGACGTTG | | | |
| | | TAAAACGACGGCCAGT | | | |
| DRIVER_ | PLV10048 | GAATTCGAGCTTGCATGCCTGCAGGTCGTTACATAACTTACGGTAAAT | 316 | N/A | |
| pCMV_R- | | GGCCCGCCTGGCTGACCGCCCAACGACCCCCGCCCATTGACGTCAAT | | | |
| U5- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| LTR_v3 | | AAGTGTATCATATGCCAAGTACGCCCCCTATTGACGTCAATGACGGTA | | | |
| | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTGAGCTTTGATCAGAATGAATTTGTCTTGGCTCCGTTT | | | |
| | | CTTCTTTCGCCCCGTCTAGATTCCTCTCTTACAGCTCGAGTGGCCTTCT | | | |
| | | CAGTCGAACCGTTCACGTTGCGAGCTGCTGGCGGCCGCAACATTTTG | | | |
| | | GCGCCAGAACTGGGACCTGAAGAATGGCAGAGAGATGCTAAGAGGA | | | |
| | | ACGCTGCTTTGGAGCTCCACAGGAAAGGATCTTCGTATCGGACATCG | | | |
| | | GAGCAACGGACAGGTACACATGCTAGCGCTAGCTTAAAATTTCAGTT | | | |
| | | TTGTAAAGTGTTGCTGAGGATGTGGTAGGATACGAATTAAGCTTGAA | | | |
| | | TCAGTGCTAACCCAACGCTGGTTCTGCTTGGGTCAGCAGCGTGTTAAT | | | |
| | | CGGAACTAGAAACGGAAACAGGCAGGTTAGCCGCAGCTTTTTAGGA | | | |
| | | AGCTGCTTAGGTGGAAGAAGAAAGGGTTTAAAGTCATGGATCAGGC | | | |
| | | GGTTGCCCATAGTTTTCAGGAGTTGTTTCAGGCCAGAGGAGTAAGGC | | | |
| | | TTGAAGTACAATTAGTAAAAAATTTTTTAGGTAAGATAGATAGCTGTT | | | |
| | | GCCCATGGTTCAAGGAAGAAGAAACACTAGATTGTGGAACCTGGGA | | | |
| | | GAAAGTTGGTGAGGCCTTAAAAATCACTCAGGCAGATAATTTTACCCT | | | |
| | | AGGCCTCTGGGCACTCATAAATGATGCAATAAAAGATGCCACTTCCCC | | | |
| | | AGGGCTAAGTTGCCCCCAGGCGGAGCTTGTGGTATCTCAGGAGGAG | | | |
| | | TGCCTGTCAGAGAGGGCCTCCTCAGAAAAAGATCTTCTTAACTCAAAA | | | |
| | | ATTGATAAATGTGGAAACTCGGATGAAAAACTGATTTTTAACAAAAA | | | |
| | | TCACTCAGATAGAGGAGCTGCCCATTACCTTAATGAGAATTGGTCCTC | | | |
| | | TTGTGAATCTCCTGCTCAACCTGTAGTCCCCACTTCGGGAGGTGCCAC | | | |
| | | TCATAGGGACACACGACTAAGCGAGTTAGAGTTTGAGATTAAGCTTC | | | |
| | | AGAGGCTGACTAATGAGCTTCGGGAACTAAAAAAGATGTCAGAAGC | | | |
| | | GGAGAAGAGTAACTCTTCTGTAGTTCACCAGGTGCCGCTAGAAAAGG | | | |
| | | TTGTGAGTCAGGCTCATGGGAAAGGACAGAATATCTCTAATACGCTA | | | |
| | | GCCTTTCCTGTGGTTGAGGTAGTTGATCAGCAAGATACTAGGGGCAG | | | |
| | | ACATTACCAGACCTTAGATTTCAAGTTGATAAAAGAGTTAAAGGCGG | | | |
| | | CTGTTGTGCAATATGGCCCTTCAGCCCCATTCACTCAAGCATTACTGG | | | |
| | | ACACAGTTGTGGAGTCACACTTAACCCCTTTAGATTGGAAGACTCTTT | | | |
| | | CTAAGGCTACCCTGTCAGGAGGAGATTTTTTGCTTTGGGATTCTGAAT | | | |
| | | GGCGAGACGCCAGTAAGAAAACTGCTGCTTCTAACGCTCAGGCTGGT | | | |
| | | AATTCAGACTGGGATAGCAACATGCTTTTAGGAGAGGGCCCTTATGA | | | |
| | | GGGACAGACAAATCAGATTGATTTTCCCGTTGCAGTGTACGCGCAAA | | | |
| | | TTGCGACGGCCGCACGCCGTGCTTGGGGAAGGTTGCCAGTCAAAGG | | | |
| | | AGAGATTGGTGGAAGTTTAGCTAGCATTCGGCAGAGTTCTGATGAAC | | | |
| | | CATATCAGGATTTTGTGGACAGGCTATTGATTTCAGCTAGTAGAATCC | | | |
| | | TTGGAAATCCGGACACGGGAAGTCCTTTCGTTATGCAATTGGCTTATG | | | |
| | | AGAATGCTAACGCAATTTGCCGAGCTGCGATTCAACCGCATAAGGGA | | | |
| | | ACGACAGATTTGGCGGGATATGTCCGTCTTTGCGCAGACATCGGGCC | | | |
| | | TTCCTGCGAGACCTTGCAGGGAACCCACGCGCAGGCAATGTTCTCTA | | | |
| | | GGAAACGAGGGAATAGTGCATGCTTTAAATGTGGAAGTTTAGATCAT | | | |
| | | TTTAGAATTGATTGTCCTCAGAACAAGGGCGCCGAGGTTAGACAAAC | | | |
| | | AGGCCGTGCCCCGGGAATATGTCCCCGATGTGGAAAGGGCCGCCACT | | | |
| | | GGGCGAAAGATTGCAAGCATAAAACGAGGGTTTTGAGCCGCCCGGT | | | |
| | | GCCGGGAAACGAGGAAAGGGGTCAGCCCCAGGCCCCAAGTTACTCA | | | |
| | | AAGAAGACAGCTTATGGGGCTCTAAATCTGCTGCCCAGCCAACAAGA | | | |
| | | TCAGTTCTTGAGCTTGTCAGGTCAAACCCAGGAAACGCAAGACTGGA | | | |
| | | CCTCTGTTCCACTGTCCATGCAGCATTAACCCCAGAAGTGGGAGTCCA | | | |
| | | AACTCTGCCTACCGGAGTCTTTGGACCACTACCTGTAGGAACCTGTGG | | | |
| | | TTTTCTCTTAGGACGAAGCAGTTCTATTGTAGAAGGCCTGCAGATTTA | | | |
| | | TCCAGGTGTTATAAGTAATGATTATGAGGGAGAAATTAAAATCATAG | | | |
| | | CCGCTTGCCCTCGTGGTGCTATAACTATACCCGCTAATCAGAAAATTG | | | |
| | | CTCAACTTACCTTGATCCCCTTGCGCTGGTCACTATCTAAATTCTCTAA | | | |
| | | AAATGAAGAAGGACAGATTAACTTTGACTCCTCTGGCGTAAATTGGG | | | |
| | | TGAAATCTATCACTAATCAGAGACCTAACCTTAAATTGATTCTTGATG | | | |
| | | GAAAAAGCTTTGAAGGATTAATAGATACCGGGGCCGATGTAACCATT | | | |
| | | ATTAGAGGGCAGGACTGGCCCTCAAACTGGCCCCTGTCTGTTTCCTTG | | | |
| | | ACTCACCTTCAAGGAATTGGTTATGCCAGTAACCCAAAACGTAGTTCC | | | |
| | | AAATTGCTAACCTGGAGAGATGAGGATGGAAAATCAGGAAATATTCA | | | |
| | | GCCGTATGTTATGCAAAATTTGCCTGTAACCCTGTGGGGAAGAGATC | | | |
| | | TGTTGTCACAGATGGGCGTTATCCTGTGCAGTTCTAAGGAAATGGTG | | | |
| | | ACTGAACAGACGTTCAGGCAGGGACCCCTGCCTGATCGTGGACTAAT | | | |
| | | AAAGAAGGGACAGAAAATTAAGACTTTTGAAGATCTTAAACCCCACT | | | |
| | | CTAACGTGAGAGGTTTAAAGTATTTTCAGTAGTGGCCGCTGTCTTGCC | | | |
| | | TGCATCCCACGCCGAAAAAATTCAATGGCGTAATGATATTCCGGTGT | | | |
| | | GGGTAGATCAGTGGTCTTTACCTAAAGAGAAAATAGAGGCCGCTTCT | | | |
| | | CTGCTAGTGCAGGAGCAGTTAGAAGCAGGACATTTGGTGGAGTCTCA | | | |
| | | TTCTCCCTGGAATACACCCATTTTCATTATCAGGAAGAAATCGGGAAA | | | |
| | | ATGGAGACTGTTGCAAGATTTAAGAAAGGTTAATGAAACCATGGTAC | | | |
| | | TTATGGGAACTTTACAACCGGGGCTCCCCTCCCCAGTAGCCATTCCTA | | | |
| | | AGGGATACTATAAGATTGTTATAGATTTGAAAGATTGTTTCTTTACCA | | | |
| | | TCCCTTTGCATCCAGAGGATTGTGAGAGATTTGCTTTTAGTGTTCCTTC | | | |
| | | TGTAAATTTCAAGGAACCCATGAAAAGATATCAATGGACAGTTCTCCC | | | |
| | | GCAGGGGATGGCTAATAGTCCCACCTTATGTCAAAAGTTTGTGGCAA | | | |
| | | AGGCAATTCAGCCTGTTAGACAACAATGGCCAAATATTTACATCATTC | | | |
| | | ATTTCACAGATGATGTTTTGATGGCGGGAAAGGACCCCCAAGATTTG | | | |
| | | CTTTTGTGTTATGGAGACTTACGAAAGGCCCTGGCTGATAAGGGATT | | | |
| | | ACAAATTGCTTCTGAAAAGATACAAACTCAGGATCCTTATAATTATTT | | | |
| | | GGGTTTTAGACTCACTGACCAAGCTGTTTTTCACCAGAAAATTGTTAT | | | |
| | | TCGTAGAGATAACTTAAGGACCTTAAATGATTTTCAAAAATTGTTAGG | | | |
| | | TGATATAAACTGGCTTCGCCCCTATCTAAAGCTTACTACAGGGGAGTT | | | |
| | | GAAACCTTTATTTGATATTCTTAAAGGGAGTTCTGATCCTACTTCCCCT | | | |
| | | AGATCCCTAACCTCAGAAGGTTTACTGGCCTTACAGCTAGTGGAAAA | | | |
| | | GGCTATTGAAGAACAGTTTGTCACTTACATAGATTACTCCCTGCCGCT | | | |
| | | GCACCTGTTAATTTTTAACACGACTCATGTGCCTACGGGATTGCTATG | | | |
| | | GCAAAAATTTCCTATAATGTGGATACATTCAAGGATTTCTCCCAAACG | | | |
| | | TAATATTTTGCCATATCATGAAGCAGTGGCTCAGATGATTATCACTGG | | | |
| | | AAGAAGGCAGGCATTGACTTATTTTGGAAAGGAGCCAGATATCATTG | | | |
| | | TCCAGCCTTACAGCGTGAGTCAGGACACTTGGCTGAAACAGCATAGT | | | |
| | | ACAGATTGGTTGCTTGCACAATTAGGGTTTGAAGGAACTATAGATAG | | | |
| | | CCACTACCCCCAAGATAGGTTGATAAAATTCTTAAATGTACATGATAT | | | |
| | | GATATTTCCTAAGATGACTTCCTTACAGCCTTTAAATAATGCTCTATTG | | | |
| | | ATTTTTACTGATGGCTCCTCTAAAGGGCGAGCTGGATATCTTATTAGT | | | |
| | | AATCAACAGGTTATCGTAGAGACTCCTGGTCTCTCGGCTCAGCTCGCC | | | |
| | | GAACTAACAGCAGTACTGAAGGTTTTTCAGTCTGTACAGGAGGCTTTT | | | |
| | | AATATTTTTACTGACAGTTTATATGTTGCTCAGTCAGTACCCTTATTGG | | | |
| | | AAACCTGTGGTACTTTTAACTTCAATACGCCGTCAGGATCTTTATTTTC | | | |
| | | AGAATTACAAAACATCATTCTCGCCCGGAAAAATCCGTTTTATATTGG | | | |
| | | CCACATACGGTCTCACTCTGGTCTTCCTGGACCTCTGGCAGAGGGTAA | | | |
| | | TAATTGCATTGACAGAGCTCTAATAGGAGAAGCCTTAGTTTCAGATCG | | | |
| | | GGTTGCTTTGGCCCAACGTGATCATGAAAGGTTTCATCTCTCTAGCCA | | | |
| | | TACCCTAAGGCTCCGACATAAGATCACCAAGGAGCAAGCGAGAATGA | | | |
| | | TTGTAAAACAATGTCCTAAATGTATTACTTTATCTCCAGTGCCGCATCT | | | |
| | | AGGAGTTAATCCTAGAGGCCTTATGCCTAATCATATTTGGCAAATGGA | | | |
| | | TATAACCCATTATGCAGAATTTGGAAAACTAAAATATATACATGTTTG | | | |
| | | CATTGATACTTGTTCAGGATTTCTCTTTGCTTCTCTGCATACAGGAGAA | | | |
| | | GCTTCAAAAAACGTAATTGATCATTGCCTACAAGCATTTAATGCCATG | | | |
| | | GGATTACCTAAACTTATTAAGACAGACAATGGGCCATCTTATTCCAGT | | | |
| | | AAAAACTTTATTTCATTCTGTAAAGAATTCGGTATTAAACATAAAACT | | | |
| | | GGAATTCCTTACAACCCCATGGGACAAGGAATAGTTGAACGTGCTCA | | | |
| | | TCGCACCTTAAAGAATTGGCTCTTTAAGACAAAAGAGGGGCAGCTAT | | | |
| | | ATCCCCCAAGGTCTCCAAAGGCCCACCTTGCCTTCACCTTATTTGTCCT | | | |
| | | AAATTTCTTGCACACCGATATCAAGGGCCAGTCTGCAGCGGATCGCC | | | |
| | | ACTGGCATCCAGTTACTTCTAATTCTTATGCATTGGTAAAATGGAAGG | | | |
| | | ACCCCCTGACTAATGAATGGAAGGGTCCAGATCCAGTTCTAATTTGG | | | |
| | | GGTAGAGGCTCAGTTTGTGTTTTTTCACGAGATGAAGATGGAGCACG | | | |
| | | GTGGCTGCCAGAGAGATTAATTCGTCAGACGAACACAGATTCTGACT | | | |
| | | CTTCTGGTAAGTATCATTCTAAAGACTAAAATTCCTTTTGTGCTTAAAA | | | |
| | | TTCAGCTGAGAGCAACAGCTCTCAAAGCTGTTCTCCAGCTACTCTCTG | | | |
| | | AGCCAGCTCCCGACAGGAGGCCGGAGACTAGCCTCAGCTTTACAATT | | | |
| | | TGCATTTAAATAAAGTACCTAGACTTCCCCGAAAAAAGTTCTGCTTTTC | | | |
| | | TACTTTCTCACTGTCTTTCAAGATTTTGTCTTTCAAGCAGGTAAATCAA | | | |
| | | CATTCTCGAGGCGGACCAGCGGATGTGCATCCCCGCCCCCCTAGAGC | | | |
| | | ACTCAGGTGGCAGCTGTTATCCCCAGTCTCAGGACATTCCAGCATGTG | | | |
| | | GCCTTCAGTCTGAGTTAAAAATTAGGTTTACCCAGAGGACTAGAATA | | | |
| | | GTAGATATTTCTATATTAATAAAGATTGGTTTTTATTTTGATAGACAGG | | | |
| | | CTTAGCCCCTTAGCTGACCTCTGGCTTTTCACCCTTGCTGTTACTGCAA | | | |
| | | GGTGTCTTTAGCTCAATAAGGCTGTGGAAAAAAACAGGGATGAGGA | | | |
| | | GGAACGGCTCCCAGCTCCTATTTTAGCCACAAATCGTGGTGTTACTAA | | | |
| | | CGACATAATTCTTGCTTAGGCTTTGCTAAATCTGAGGTTGATAATTCTC | | | |
| | | CTTTAGGAGCTGCACAGCGCTCAGAACTGTGCATACTGATTTGTGATG | | | |
| | | GTACAAATTCAGTATGGGCATCGCTTGGTGCAGATGGAGGTACTGCA | | | |
| | | AGGAAAGGTCCCAGCTTGACCATTTCTGAGTTTCCTGTGAGATAAACC | | | |
| | | CGGTTTGAAAGAGGTTGGTACCAAATTATATATCCCTCGGCTCTACCT | | | |
| | | CGCCTCCCCAAAAGGTACCAGAGCCACAGGTGTGGATTTTAACAGAA | | | |
| | | TCCACGGGAGGAATCGGGTCCATGTCCACCCAAGCCAAGGTTAAAAG | | | |
| | | CCCACTCATCTACGGATGAGAAAATCATTTGATCACCTCAGTTAAGCG | | | |
| | | CTGCCTTATTTTAACTTAATTAATAGGGGGGAGAGAGATTGGAGACT | | | |
| | | TACTATTGAAAGGGCAAGCCCTTCACTGCCTCCCACCCAAATAAAAAA | | | |
| | | GCCAATTGGCCTTGTACTACAGAGCTGGCCGGACCCCTTATCCCTGTT | | | |
| | | ACCCACCAATCATCCAAAAATGCGGAGGAATATCAACTTAGTGTTATT | | | |
| | | CTTATTATAGTGTATTTCACACTTGTTCAGTCAAACTTAGCCAGAGTTC | | | |
| | | CAACGCCCTACTTAAAATTCAACTAGAAAGTTACCTACCAAGTACTAA | | | |
| | | TTAGCATTATAAAGTCAGAGCCTACAGCTCCAGGCTTTTCAGTTAGTT | | | |
| | | GTTTACTAAGATAAGAAAAGACAGTCTTAGCCAGATACAGTTTACCAT | | | |
| | | AATAAAAGTTAAAGAATCCCAGGGAAGCAAGTTTTTTCTTTTAGCCCT | | | |
| | | AGATTCCAGGCAGAACTATTGAGCATAGATAATTTTTCCCCCTCAGGC | | | |
| | | CAGCTTTTTCTTTTTTTTTAATTTTGTTAATAAAAGGGAGGAGATGTAG | | | |
| | | TCTCCCCTCCCCCAGCCTGAAACCTGCTTGCTCAGGGGTGGAGCTTCC | | | |
| | | CGCTCATCGCTCTGCCACGCCCACTGCTGGAACCTGCGGAGCCACAC | | | |
| | | ACGTGCACCTTTCTACTGGACCAGAGATTATTCGGCGGGAATCGGGT | | | |
| | | CCCCTCCCCCTTCCTTCATAACTAGTGTCCCAACAATAAAATTTGAGCT | | | |
| | | TTGATCAGAATGAATTTGTCTTGGCTCCGTTTCTTCTTTCGCCCCGTCT | | | |
| | | AGATTCCTCTCTTACAGCTCGAGTGGCCTTCTCAGTCGAACCGTTCAC | | | |
| | | GTTGCGAGCTGCTGGCGGCCGCAACAGGGGATCCAGACATGATAAG | | | |
| | | ATACATTGATGAGTTTGGACAAACCACAACTAGAATGCAGTGAAAAA | | | |
| | | AATGCTTTATTTGTGAAATTTGTGATGCTATTGCTTTATTTGTAACCAT | | | |
| | | TATAAGCTGCAATAAACAAGTTAACAACAACAATTGCATTCATTTTAT | | | |
| | | GTTTCAGGTTCAGGGGGAGGTGTGGGAGGTTTTTTCGGATCCTCTAG | | | |
| | | AGTCGACCTGCAGGCATGCAAGCTTGGCGTAATCATGGTCATAGCTG | | | |
| | | TTTCCTGTGTGAAATTGTTATCCGCTCACAATTCCACACAACATACGA | | | |
| | | GCCGGAAGCATAAAGTGTAAAGCCTGGGGTGCCTAATGAGTGAGCT | | | |
| | | AACTCACATTAATTGCGTTGCGCTCACTGCCCGCTTTCCAGTCGGGAA | | | |
| | | ACCTGTCGTGCCAGCTGCATTAATGAATCGGCCAACGCGCGGGGAGA | | | |
| | | GGCGGTTTGCGTATTGGGCGCTCTTCCGCTTCCTCGCTCACTGACTCG | | | |
| | | CTGCGCTCGGTCGTTCGGCTGCGGCGAGCGGTATCAGCTCACTCAAA | | | |
| | | GGCGGTAATACGGTTATCCACAGAATCAGGGGATAACGCAGGAAAG | | | |
| | | AACATGTGAGCAAAAGGCCAGCAAAAGGCCAGGAACCGTAAAAAGG | | | |
| | | CCGCGTTGCTGGCGTTTTTCCATAGGCTCCGCCCCCCTGACGAGCATC | | | |
| | | ACAAAAATCGACGCTCAAGTCAGAGGTGGCGAAACCCGACAGGACT | | | |
| | | ATAAAGATACCAGGCGTTTCCCCCTGGAAGCTCCCTCGTGCGCTCTCC | | | |
| | | TGTTCCGACCCTGCCGCTTACCGGATACCTGTCCGCCTTTCTCCCTTCG | | | |
| | | GGAAGCGTGGCGCTTTCTCATAGCTCACGCTGTAGGTATCTCAGTTCG | | | |
| | | GTGTAGGTCGTTCGCTCCAAGCTGGGCTGTGTGCACGAACCCCCCGT | | | |
| | | TCAGCCCGACCGCTGCGCCTTATCCGGTAACTATCGTCTTGAGTCCAA | | | |
| | | CCCGGTAAGACACGACTTATCGCCACTGGCAGCAGCCACTGGTAACA | | | |
| | | GGATTAGCAGAGCGAGGTATGTAGGCGGTGCTACAGAGTTCTTGAA | | | |
| | | GTGGTGGCCTAACTACGGCTACACTAGAAGGACAGTATTTGGTATCT | | | |
| | | GCGCTCTGCTGAAGCCAGTTACCTTCGGAAAAAGAGTTGGTAGCTCT | | | |
| | | TGATCCGGCAAACAAACCACCGCTGGTAGCGGTGGTTTTTTTGTTTGC | | | |
| | | AAGCAGCAGATTACGCGCAGAAAAAAAGGATCTCAAGAAGATCCTTT | | | |
| | | GATCTTTTCTACGGGGTCTGACGCTCAGTGGAACGAAAACTCACGTTA | | | |
| | | AGGGATTTTGGTCATGAGATTATCAAAAAGGATCTTCACCTAGATCCT | | | |
| | | TTTAAATTAAAAATGAAGTTTTAAATCAATCTAAAGTATATATGAGTA | | | |
| | | AACTTGGTCTGACAGTTACCAATGCTTAATCAGTGAGGCACCTATCTC | | | |
| | | AGCGATCTGTCTATTTCGTTCATCCATAGTTGCCTGACTCCCCGTCGTG | | | |
| | | TAGATAACTACGATACGGGAGGGCTTACCATCTGGCCCCAGTGCTGC | | | |
| | | AATGATACCGCGAGACCCACGCTCACCGGCTCCAGATTTATCAGCAAT | | | |
| | | AAACCAGCCAGCCGGAAGGGCCGAGCGCAGAAGTGGTCCTGCAACT | | | |
| | | TTATCCGCCTCCATCCAGTCTATTAATTGTTGCCGGGAAGCTAGAGTA | | | |
| | | AGTAGTTCGCCAGTTAATAGTTTGCGCAACGTTGTTGCCATTGCTACA | | | |
| | | GGCATCGTGGTGTCACGCTCGTCGTTTGGTATGGCTTCATTCAGCTCC | | | |
| | | GGTTCCCAACGATCAAGGCGAGTTACATGATCCCCCATGTTGTGCAA | | | |
| | | AAAAGCGGTTAGCTCCTTCGGTCCTCCGATCGTTGTCAGAAGTAAGTT | | | |
| | | GGCCGCAGTGTTATCACTCATGGTTATGGCAGCACTGCATAATTCTCT | | | |
| | | TACTGTCATGCCATCCGTAAGATGCTTTTCTGTGACTGGTGAGTACTC | | | |
| | | AACCAAGTCATTCTGAGAATAGTGTATGCGGCGACCGAGTTGCTCTT | | | |
| | | GCCCGGCGTCAATACGGGATAATACCGCGCCACATAGCAGAACTTTA | | | |
| | | AAAGTGCTCATCATTGGAAAACGTTCTTCGGGGCGAAAACTCTCAAG | | | |
| | | GATCTTACCGCTGTTGAGATCCAGTTCGATGTAACCCACTCGTGCACC | | | |
| | | CAACTGATCTTCAGCATCTTTTACTTTCACCAGCGTTTCTGGGTGAGCA | | | |
| | | AAAACAGGAAGGCAAAATGCCGCAAAAAAGGGAATAAGGGCGACA | | | |
| | | CGGAAATGTTGAATACTCATACTCTTCCTTTTTCAATATTATTGAAGCA | | | |
| | | TTTATCAGGGTTATTGTCTCATGAGCGGATACATATTTGAATGTATTT | | | |
| | | AGAAAAATAAACAAATAGGGGTTCCGCGCACATTTCCCCGAAAAGTG | | | |
| | | CCACCTGACGTCTAAGAAACCATTATTATCATGACATTAACCTATAAA | | | |
| | | AATAGGCGTATCACGAGGCCCTTTCGTCTCGCGCGTTTCGGTGATGA | | | |
| | | CGGTGAAAACCTCTGACACATGCAGCTCCCGGAGACGGTCACAGCTT | | | |
| | | GTCTGTAAGCGGATGCCGGGAGCAGACAAGCCCGTCAGGGCGCGTC | | | |
| | | AGCGGGTGTTGGCGGGTGTCGGGGCTGGCTTAACTATGCGGCATCA | | | |
| | | GAGCAGATTGTACTGAGAGTGCACCATATGCGGTGTGAAATACCGCA | | | |
| | | CAGATGCGTAAGGAGAAAATACCGCATCAGGCGCCATTCGCCATTCA | | | |
| | | GGCTGCGCAACTGTTGGGAAGGGCGATCGGTGCGGGCCTCTTCGCT | | | |
| | | ATTACGCCAGCTGGCGAAAGGGGGATGTGCTGCAAGGCGATTAAGT | | | |
| | | TGGGTAACGCCAGGGTTTTCCCAGTCACGACGTTGTAAAACGACGGC | | | |
| | | CAGT | | | |
| DRIVER_ | PLV10049 | GAATTCGAGCTTGCATGCCTGCAGGTCGTTACATAACTTACGGTAAAT | 317 | N/A | |
| pCMV_R- | | GGCCCGCCTGGCTGACCGCCCAACGACCCCCGCCCATTGACGTCAAT | | | |
| U5- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| delPol- | | AAGTGTATCATATGCCAAGTACGCCCCCTATTGACGTCAATGACGGTA | | | |
| LTR_v1 | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGAGCTTTGATCAGAATGAA | | | |
| | | TTTGTCTTGGCTCCGTTTCTTCTTTCGCCCCGTCTAGATTCCTCTCTTAC | | | |
| | | AGCTCGAGTGGCCTTCTCAGTCGAACCGTTCACGTTGCGAGCTGCTG | | | |
| | | GCGGCCGCAACATTTTGGCGCCAGAACTGGGACCTGAAGAATGGCA | | | |
| | | GAGAGATGCTAAGAGGAACGCTGCTTTGGAGCTCCACAGGAAAGGA | | | |
| | | TCTTCGTATCGGACATCGGAGCAACGGACAGGTACACATGCTAGCGC | | | |
| | | TAGCTTAAAATTTCAGTTTTGTAAAGTGTTGCTGAGGATGTGGTAGG | | | |
| | | ATACGAATTAAGCTTGAATCAGTGCTAACCCAACGCTGGTTCTGCTTG | | | |
| | | GGTCAGCAGCGTGTTAATCGGAACTAGAAACGGAAACAGGCAGGTT | | | |
| | | AGCCGCAGCTTTTTAGGAAGCTGCTTAGGTGGAAGAAGAAAGGGTTT | | | |
| | | AAAGTCATGGATCAGGCGGTTGCCCATAGTTTTCAGGAGTTGTTTCA | | | |
| | | GGCCAGAGGAGTAAGGCTTGAAGTACAATTAGTAAAAAATTTTTTAG | | | |
| | | GTAAGATAGATAGCTGTTGCCCATGGTTCAAGGAAGAAGAAACACTA | | | |
| | | GATTGTGGAACCTGGGAGAAAGTTGGTGAGGCCTTAAAAATCACTCA | | | |
| | | GGCAGATAATTTTACCCTAGGCCTCTGGGCACTCATAAATGATGCAAT | | | |
| | | AAAAGATGCCACTTCCCCAGGGCTAAGTTGCCCCCAGGCGGAGCTTG | | | |
| | | TGGTATCTCAGGAGGAGTGCCTGTCAGAGAGGGCCTCCTCAGAAAAA | | | |
| | | GATCTTCTTAACTCAAAAATTGATAAATGTGGAAACTCGGATGAAAA | | | |
| | | ACTGATTTTTAACAAAAATCACTCAGATAGAGGAGCTGCCCATTACCT | | | |
| | | TAATGAGAATTGGTCCTCTTGTGAATCTCCTGCTCAACCTGTAGTCCC | | | |
| | | CACTTCGGGAGGTGCCACTCATAGGGACACACGACTAAGCGAGTTAG | | | |
| | | AGTTTGAGATTAAGCTTCAGAGGCTGACTAATGAGCTTCGGGAACTA | | | |
| | | AAAAAGATGTCAGAAGCGGAGAAGAGTAACTCTTCTGTAGTTCACCA | | | |
| | | GGTGCCGCTAGAAAAGGTTGTGAGTCAGGCTCATGGGAAAGGACAG | | | |
| | | AATATCTCTAATACGCTAGCCTTTCCTGTGGTTGAGGTAGTTGATCAG | | | |
| | | CAAGATACTAGGGGCAGACATTACCAGACCTTAGATTTCAAGTTGAT | | | |
| | | AAAAGAGTTAAAGGCGGCTGTTGTGCAATATGGCCCTTCAGCCCCAT | | | |
| | | TCACTCAAGCATTACTGGACACAGTTGTGGAGTCACACTTAACCCCTT | | | |
| | | TAGATTGGAAGACTCTTTCTAAGGCTACCCTGTCAGGAGGAGATTTTT | | | |
| | | TGCTTTGGGATTCTGAATGGCGAGACGCCAGTAAGAAAACTGCTGCT | | | |
| | | TCTAACGCTCAGGCTGGTAATTCAGACTGGGATAGCAACATGCTTTTA | | | |
| | | GGAGAGGGCCCTTATGAGGGACAGACAAATCAGATTGATTTTCCCGT | | | |
| | | TGCAGTGTACGCGCAAATTGCGACGGCCGCACGCCGTGCTTGGGGA | | | |
| | | AGGTTGCCAGTCAAAGGAGAGATTGGTGGAAGTTTAGCTAGCATTCG | | | |
| | | GCAGAGTTCTGATGAACCATATCAGGATTTTGTGGACAGGCTATTGA | | | |
| | | TTTCAGCTAGTAGAATCCTTGGAAATCCGGACACGGGAAGTCCTTTCG | | | |
| | | TTATGCAATTGGCTTATGAGAATGCTAACGCAATTTGCCGAGCTGCGA | | | |
| | | TTCAACCGCATAAGGGAACGACAGATTTGGCGGGATATGTCCGTCTT | | | |
| | | TGCGCAGACATCGGGCCTTCCTGCGAGACCTTGCAGGGAACCCACGC | | | |
| | | GCAGGCAATGTTCTCTAGGAAACGAGGGAATAGTGCATGCTTTAAAT | | | |
| | | GTGGAAGTTTAGATCATTTTAGAATTGATTGTCCTCAGAACAAGGGC | | | |
| | | GCCGAGGTTAGACAAACAGGCCGTGCCCCGGGAATATGTCCCCGATG | | | |
| | | TGGAAAGGGCCGCCACTGGGCGAAAGATTGCAAGCATAAAACGAGG | | | |
| | | GTTTTGAGCCGCCCGGTGCCGGGAAACGAGGAAAGGGGTCAGCCCC | | | |
| | | AGGCCCCAAGTTACTCAAAGAAGACAGCTTATGGGGCTCTAAATCTG | | | |
| | | CTGCCCAGCCAACAAGATCAGTTCTTGAGCTTGTCAGGTCAAACCCAG | | | |
| | | GAAACGCAAGACTGGACCTCTGTTCCACTGTCCATGCAGCATTAACCC | | | |
| | | CAGAAGTGGGAGTCCAAACTCTGCCTACCGGAGTCTTTGGACCACTA | | | |
| | | CCTGTAGGAACCTGTGGTTTTCTCTTAGGACGAAGCAGTTCTATTGTA | | | |
| | | GAAGGCCTGCAGATTTATCCAGGTGTTATAAGTAATGATTATGAGGG | | | |
| | | AGAAATTAAAATCATAGCCGCTTGCCCTCGTGGTGCTATAACTATACC | | | |
| | | CGCTAATCAGAAAATTGCTCAACTTACCTTGATCCCCTTGCGCTGGTC | | | |
| | | ACTATCTAAATTCTCTAAAAATGAAGAAGGACAGATTAACTTTGACTC | | | |
| | | CTCTGGCGTAAATTGGGTGAAATCTATCACTAATCAGAGACCTAACCT | | | |
| | | TAAATTGATTCTTGATGGAAAAAGCTTTGAAGGATTAATAGATACCG | | | |
| | | GGGCCGATGTAACCATTATTAGAGGGCAGGACTGGCCCTCAAACTGG | | | |
| | | CCCCTGTCTGTTTCCTTGACTCACCTTCAAGGAATTGGTTATGCCAGTA | | | |
| | | ACCCAAAACGTAGTTCCAAATTGCTAACCTGGAGAGATGAGGATGGA | | | |
| | | AAATCAGGAAATATTCAGCCGTATGTTATGCAAAATTTGCCTGTAACC | | | |
| | | CTGTGGGGAAGAGATCTGTTGTCACAGATGGGCGTTATCCTGTGCAG | | | |
| | | TTCTAAGGAAATGGTGACTGAACAGACGTTCAGGCAGGGACCCCTGC | | | |
| | | CTGATCGTGGACTAATAAAGAAGGGACAGAAAATTAAGACTTTTGAA | | | |
| | | GATCTTAAACCCCACTCTAACGTGAGAGGTTTAAAGTATTTTCAGTAG | | | |
| | | TGTAAAATTCCTTTTGTGCTTAAAATTCAGCTGAGAGCAACAGCTCTC | | | |
| | | AAAGCTGTTCTCCAGCTACTCTCTGAGCCAGCTCCCGACAGGAGGCC | | | |
| | | GGAGACTAGCCTCAGCTTTACAATTTGCATTTAAATAAAGTACCTAGA | | | |
| | | CTTCCCCGAAAAAAGTTCTGCTTTTCTACTTTCTCACTGTCTTTCAAGA | | | |
| | | TTTTGTCTTTCAAGCAGGTAAATCAACATTCTCGAGGCGGACCAGCGG | | | |
| | | ATGTGCATCCCCGCCCCCCTAGAGCACTCAGGTGGCAGCTGTTATCCC | | | |
| | | CAGTCTCAGGACATTCCAGCATGTGGCCTTCAGTCTGAGTTAAAAATT | | | |
| | | AGGTTTACCCAGAGGACTAGAATAGTAGATATTTCTATATTAATAAAG | | | |
| | | ATTGGTTTTTATTTTGATAGACAGGCTTAGCCCCTTAGCTGACCTCTG | | | |
| | | GCTTTTCACCCTTGCTGTTACTGCAAGGTGTCTTTAGCTCAATAAGGCT | | | |
| | | GTGGAAAAAAACAGGGATGAGGAGGAACGGCTCCCAGCTCCTATTTT | | | |
| | | AGCCACAAATCGTGGTGTTACTAACGACATAATTCTTGCTTAGGCTTT | | | |
| | | GCTAAATCTGAGGTTGATAATTCTCCTTTAGGAGCTGCACAGCGCTCA | | | |
| | | GAACTGTGCATACTGATTTGTGATGGTACAAATTCAGTATGGGCATC | | | |
| | | GCTTGGTGCAGATGGAGGTACTGCAAGGAAAGGTCCCAGCTTGACC | | | |
| | | ATTTCTGAGTTTCCTGTGAGATAAACCCGGTTTGAAAGAGGTTGGTAC | | | |
| | | CAAATTATATATCCCTCGGCTCTACCTCGCCTCCCCAAAAGGTACCAG | | | |
| | | AGCCACAGGTGTGGATTTTAACAGAATCCACGGGAGGAATCGGGTCC | | | |
| | | ATGTCCACCCAAGCCAAGGTTAAAAGCCCACTCATCTACGGATGAGA | | | |
| | | AAATCATTTGATCACCTCAGTTAAGCGCTGCCTTATTTTAACTTAATTA | | | |
| | | ATAGGGGGGAGAGAGATTGGAGACTTACTATTGAAAGGGCAAGCCC | | | |
| | | TTCACTGCCTCCCACCCAAATAAAAAAGCCAATTGGCCTTGTACTACA | | | |
| | | GAGCTGGCCGGACCCCTTATCCCTGTTACCCACCAATCATCCAAAAAT | | | |
| | | GCGGAGGAATATCAACTTAGTGTTATTCTTATTATAGTGTATTTCACA | | | |
| | | CTTGTTCAGTCAAACTTAGCCAGAGTTCCAACGCCCTACTTAAAATTC | | | |
| | | AACTAGAAAGTTACCTACCAAGTACTAATTAGCATTATAAAGTCAGAG | | | |
| | | CCTACAGCTCCAGGCTTTTCAGTTAGTTGTTTACTAAGATAAGAAAAG | | | |
| | | ACAGTCTTAGCCAGATACAGTTTACCATAATAAAAGTTAAAGAATCCC | | | |
| | | AGGGAAGCAAGTTTTTTCTTTTAGCCCTAGATTCCAGGCAGAACTATT | | | |
| | | GAGCATAGATAATTTTTCCCCCTCAGGCCAGCTTTTTCTTTTTTTTTAAT | | | |
| | | TTTGTTAATAAAAGGGAGGAGATGTAGTCTCCCCTCCCCCAGCCTGAA | | | |
| | | ACCTGCTTGCTCAGGGGTGGAGCTTCCCGCTCATCGCTCTGCCACGCC | | | |
| | | CACTGCTGGAACCTGCGGAGCCACACACGTGCACCTTTCTACTGGACC | | | |
| | | AGAGATTATTCGGCGGGAATCGGGTCCCCTCCCCCTTCCTTCATAACT | | | |
| | | AGTGTCCCAACAATAAAATTTGAGCTTTGATCAGAATGAATTTGTCTT | | | |
| | | GGCTCCGTTTCTTCTTTCGCCCCGTCTAGATTCCTCTCTTACAGCTCGA | | | |
| | | GTGGCCTTCTCAGTCGAACCGTTCACGTTGCGAGCTGCTGGCGGCCG | | | |
| | | CAACAGGGGATCCAGACATGATAAGATACATTGATGAGTTTGGACAA | | | |
| | | ACCACAACTAGAATGCAGTGAAAAAAATGCTTTATTTGTGAAATTTGT | | | |
| | | GATGCTATTGCTTTATTTGTAACCATTATAAGCTGCAATAAACAAGTT | | | |
| | | AACAACAACAATTGCATTCATTTTATGTTTCAGGTTCAGGGGGAGGTG | | | |
| | | TGGGAGGTTTTTTCGGATCCTCTAGAGTCGACCTGCAGGCATGCAAG | | | |
| | | CTTGGCGTAATCATGGTCATAGCTGTTTCCTGTGTGAAATTGTTATCC | | | |
| | | GCTCACAATTCCACACAACATACGAGCCGGAAGCATAAAGTGTAAAG | | | |
| | | CCTGGGGTGCCTAATGAGTGAGCTAACTCACATTAATTGCGTTGCGCT | | | |
| | | CACTGCCCGCTTTCCAGTCGGGAAACCTGTCGTGCCAGCTGCATTAAT | | | |
| | | GAATCGGCCAACGCGCGGGGAGAGGCGGTTTGCGTATTGGGCGCTC | | | |
| | | TTCCGCTTCCTCGCTCACTGACTCGCTGCGCTCGGTCGTTCGGCTGCG | | | |
| | | GCGAGCGGTATCAGCTCACTCAAAGGCGGTAATACGGTTATCCACAG | | | |
| | | AATCAGGGGATAACGCAGGAAAGAACATGTGAGCAAAAGGCCAGCA | | | |
| | | AAAGGCCAGGAACCGTAAAAAGGCCGCGTTGCTGGCGTTTTTCCATA | | | |
| | | GGCTCCGCCCCCCTGACGAGCATCACAAAAATCGACGCTCAAGTCAG | | | |
| | | AGGTGGCGAAACCCGACAGGACTATAAAGATACCAGGCGTTTCCCCC | | | |
| | | TGGAAGCTCCCTCGTGCGCTCTCCTGTTCCGACCCTGCCGCTTACCGG | | | |
| | | ATACCTGTCCGCCTTTCTCCCTTCGGGAAGCGTGGCGCTTTCTCATAG | | | |
| | | CTCACGCTGTAGGTATCTCAGTTCGGTGTAGGTCGTTCGCTCCAAGCT | | | |
| | | GGGCTGTGTGCACGAACCCCCCGTTCAGCCCGACCGCTGCGCCTTATC | | | |
| | | CGGTAACTATCGTCTTGAGTCCAACCCGGTAAGACACGACTTATCGCC | | | |
| | | ACTGGCAGCAGCCACTGGTAACAGGATTAGCAGAGCGAGGTATGTA | | | |
| | | GGCGGTGCTACAGAGTTCTTGAAGTGGTGGCCTAACTACGGCTACAC | | | |
| | | TAGAAGGACAGTATTTGGTATCTGCGCTCTGCTGAAGCCAGTTACCTT | | | |
| | | CGGAAAAAGAGTTGGTAGCTCTTGATCCGGCAAACAAACCACCGCTG | | | |
| | | GTAGCGGTGGTTTTTTTGTTTGCAAGCAGCAGATTACGCGCAGAAAA | | | |
| | | AAAGGATCTCAAGAAGATCCTTTGATCTTTTCTACGGGGTCTGACGCT | | | |
| | | CAGTGGAACGAAAACTCACGTTAAGGGATTTTGGTCATGAGATTATC | | | |
| | | AAAAAGGATCTTCACCTAGATCCTTTTAAATTAAAAATGAAGTTTTAA | | | |
| | | ATCAATCTAAAGTATATATGAGTAAACTTGGTCTGACAGTTACCAATG | | | |
| | | CTTAATCAGTGAGGCACCTATCTCAGCGATCTGTCTATTTCGTTCATCC | | | |
| | | ATAGTTGCCTGACTCCCCGTCGTGTAGATAACTACGATACGGGAGGG | | | |
| | | CTTACCATCTGGCCCCAGTGCTGCAATGATACCGCGAGACCCACGCTC | | | |
| | | ACCGGCTCCAGATTTATCAGCAATAAACCAGCCAGCCGGAAGGGCCG | | | |
| | | AGCGCAGAAGTGGTCCTGCAACTTTATCCGCCTCCATCCAGTCTATTA | | | |
| | | ATTGTTGCCGGGAAGCTAGAGTAAGTAGTTCGCCAGTTAATAGTTTG | | | |
| | | CGCAACGTTGTTGCCATTGCTACAGGCATCGTGGTGTCACGCTCGTCG | | | |
| | | TTTGGTATGGCTTCATTCAGCTCCGGTTCCCAACGATCAAGGCGAGTT | | | |
| | | ACATGATCCCCCATGTTGTGCAAAAAAGCGGTTAGCTCCTTCGGTCCT | | | |
| | | CCGATCGTTGTCAGAAGTAAGTTGGCCGCAGTGTTATCACTCATGGTT | | | |
| | | ATGGCAGCACTGCATAATTCTCTTACTGTCATGCCATCCGTAAGATGC | | | |
| | | TTTTCTGTGACTGGTGAGTACTCAACCAAGTCATTCTGAGAATAGTGT | | | |
| | | ATGCGGCGACCGAGTTGCTCTTGCCCGGCGTCAATACGGGATAATAC | | | |
| | | CGCGCCACATAGCAGAACTTTAAAAGTGCTCATCATTGGAAAACGTTC | | | |
| | | TTCGGGGCGAAAACTCTCAAGGATCTTACCGCTGTTGAGATCCAGTTC | | | |
| | | GATGTAACCCACTCGTGCACCCAACTGATCTTCAGCATCTTTTACTTTC | | | |
| | | ACCAGCGTTTCTGGGTGAGCAAAAACAGGAAGGCAAAATGCCGCAA | | | |
| | | AAAAGGGAATAAGGGCGACACGGAAATGTTGAATACTCATACTCTTC | | | |
| | | CTTTTTCAATATTATTGAAGCATTTATCAGGGTTATTGTCTCATGAGCG | | | |
| | | GATACATATTTGAATGTATTTAGAAAAATAAACAAATAGGGGTTCCG | | | |
| | | CGCACATTTCCCCGAAAAGTGCCACCTGACGTCTAAGAAACCATTATT | | | |
| | | ATCATGACATTAACCTATAAAAATAGGCGTATCACGAGGCCCTTTCGT | | | |
| | | CTCGCGCGTTTCGGTGATGACGGTGAAAACCTCTGACACATGCAGCT | | | |
| | | CCCGGAGACGGTCACAGCTTGTCTGTAAGCGGATGCCGGGAGCAGA | | | |
| | | CAAGCCCGTCAGGGCGCGTCAGCGGGTGTTGGCGGGTGTCGGGGCT | | | |
| | | GGCTTAACTATGCGGCATCAGAGCAGATTGTACTGAGAGTGCACCAT | | | |
| | | ATGCGGTGTGAAATACCGCACAGATGCGTAAGGAGAAAATACCGCA | | | |
| | | TCAGGCGCCATTCGCCATTCAGGCTGCGCAACTGTTGGGAAGGGCGA | | | |
| | | TCGGTGCGGGCCTCTTCGCTATTACGCCAGCTGGCGAAAGGGGGATG | | | |
| | | TGCTGCAAGGCGATTAAGTTGGGTAACGCCAGGGTTTTCCCAGTCAC | | | |
| | | GACGTTGTAAAACGACGGCCAGT | | | |
| DRIVER_ | PLV10050 | GAATTCGAGCTTGCATGCCTGCAGGTCGTTACATAACTTACGGTAAAT | 318 | N/A | |
| pCMV_R- | | GGCCCGCCTGGCTGACCGCCCAACGACCCCCGCCCATTGACGTCAAT | | | |
| U5- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| delPol- | | AAGTGTATCATATGCCAAGTACGCCCCCTATTGACGTCAATGACGGTA | | | |
| LTR_v2 | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGCTTTGATCAGAATGAATTT | | | |
| | | GTCTTGGCTCCGTTTCTTCTTTCGCCCCGTCTAGATTCCTCTCTTACAGC | | | |
| | | TCGAGTGGCCTTCTCAGTCGAACCGTTCACGTTGCGAGCTGCTGGCG | | | |
| | | GCCGCAACATTTTGGCGCCAGAACTGGGACCTGAAGAATGGCAGAG | | | |
| | | AGATGCTAAGAGGAACGCTGCTTTGGAGCTCCACAGGAAAGGATCTT | | | |
| | | CGTATCGGACATCGGAGCAACGGACAGGTACACATGCTAGCGCTAGC | | | |
| | | TTAAAATTTCAGTTTTGTAAAGTGTTGCTGAGGATGTGGTAGGATAC | | | |
| | | GAATTAAGCTTGAATCAGTGCTAACCCAACGCTGGTTCTGCTTGGGTC | | | |
| | | AGCAGCGTGTTAATCGGAACTAGAAACGGAAACAGGCAGGTTAGCC | | | |
| | | GCAGCTTTTTAGGAAGCTGCTTAGGTGGAAGAAGAAAGGGTTTAAA | | | |
| | | GTCATGGATCAGGCGGTTGCCCATAGTTTTCAGGAGTTGTTTCAGGC | | | |
| | | CAGAGGAGTAAGGCTTGAAGTACAATTAGTAAAAAATTTTTTAGGTA | | | |
| | | AGATAGATAGCTGTTGCCCATGGTTCAAGGAAGAAGAAACACTAGAT | | | |
| | | TGTGGAACCTGGGAGAAAGTTGGTGAGGCCTTAAAAATCACTCAGGC | | | |
| | | AGATAATTTTACCCTAGGCCTCTGGGCACTCATAAATGATGCAATAAA | | | |
| | | AGATGCCACTTCCCCAGGGCTAAGTTGCCCCCAGGCGGAGCTTGTGG | | | |
| | | TATCTCAGGAGGAGTGCCTGTCAGAGAGGGCCTCCTCAGAAAAAGAT | | | |
| | | CTTCTTAACTCAAAAATTGATAAATGTGGAAACTCGGATGAAAAACTG | | | |
| | | ATTTTTAACAAAAATCACTCAGATAGAGGAGCTGCCCATTACCTTAAT | | | |
| | | GAGAATTGGTCCTCTTGTGAATCTCCTGCTCAACCTGTAGTCCCCACTT | | | |
| | | CGGGAGGTGCCACTCATAGGGACACACGACTAAGCGAGTTAGAGTTT | | | |
| | | GAGATTAAGCTTCAGAGGCTGACTAATGAGCTTCGGGAACTAAAAAA | | | |
| | | GATGTCAGAAGCGGAGAAGAGTAACTCTTCTGTAGTTCACCAGGTGC | | | |
| | | CGCTAGAAAAGGTTGTGAGTCAGGCTCATGGGAAAGGACAGAATAT | | | |
| | | CTCTAATACGCTAGCCTTTCCTGTGGTTGAGGTAGTTGATCAGCAAGA | | | |
| | | TACTAGGGGCAGACATTACCAGACCTTAGATTTCAAGTTGATAAAAG | | | |
| | | AGTTAAAGGCGGCTGTTGTGCAATATGGCCCTTCAGCCCCATTCACTC | | | |
| | | AAGCATTACTGGACACAGTTGTGGAGTCACACTTAACCCCTTTAGATT | | | |
| | | GGAAGACTCTTTCTAAGGCTACCCTGTCAGGAGGAGATTTTTTGCTTT | | | |
| | | GGGATTCTGAATGGCGAGACGCCAGTAAGAAAACTGCTGCTTCTAAC | | | |
| | | GCTCAGGCTGGTAATTCAGACTGGGATAGCAACATGCTTTTAGGAGA | | | |
| | | GGGCCCTTATGAGGGACAGACAAATCAGATTGATTTTCCCGTTGCAG | | | |
| | | TGTACGCGCAAATTGCGACGGCCGCACGCCGTGCTTGGGGAAGGTT | | | |
| | | GCCAGTCAAAGGAGAGATTGGTGGAAGTTTAGCTAGCATTCGGCAG | | | |
| | | AGTTCTGATGAACCATATCAGGATTTTGTGGACAGGCTATTGATTTCA | | | |
| | | GCTAGTAGAATCCTTGGAAATCCGGACACGGGAAGTCCTTTCGTTAT | | | |
| | | GCAATTGGCTTATGAGAATGCTAACGCAATTTGCCGAGCTGCGATTC | | | |
| | | AACCGCATAAGGGAACGACAGATTTGGCGGGATATGTCCGTCTTTGC | | | |
| | | GCAGACATCGGGCCTTCCTGCGAGACCTTGCAGGGAACCCACGCGCA | | | |
| | | GGCAATGTTCTCTAGGAAACGAGGGAATAGTGCATGCTTTAAATGTG | | | |
| | | GAAGTTTAGATCATTTTAGAATTGATTGTCCTCAGAACAAGGGCGCC | | | |
| | | GAGGTTAGACAAACAGGCCGTGCCCCGGGAATATGTCCCCGATGTG | | | |
| | | GAAAGGGCCGCCACTGGGCGAAAGATTGCAAGCATAAAACGAGGGT | | | |
| | | TTTGAGCCGCCCGGTGCCGGGAAACGAGGAAAGGGGTCAGCCCCAG | | | |
| | | GCCCCAAGTTACTCAAAGAAGACAGCTTATGGGGCTCTAAATCTGCT | | | |
| | | GCCCAGCCAACAAGATCAGTTCTTGAGCTTGTCAGGTCAAACCCAGG | | | |
| | | AAACGCAAGACTGGACCTCTGTTCCACTGTCCATGCAGCATTAACCCC | | | |
| | | AGAAGTGGGAGTCCAAACTCTGCCTACCGGAGTCTTTGGACCACTAC | | | |
| | | CTGTAGGAACCTGTGGTTTTCTCTTAGGACGAAGCAGTTCTATTGTAG | | | |
| | | AAGGCCTGCAGATTTATCCAGGTGTTATAAGTAATGATTATGAGGGA | | | |
| | | GAAATTAAAATCATAGCCGCTTGCCCTCGTGGTGCTATAACTATACCC | | | |
| | | GCTAATCAGAAAATTGCTCAACTTACCTTGATCCCCTTGCGCTGGTCA | | | |
| | | CTATCTAAATTCTCTAAAAATGAAGAAGGACAGATTAACTTTGACTCC | | | |
| | | TCTGGCGTAAATTGGGTGAAATCTATCACTAATCAGAGACCTAACCTT | | | |
| | | AAATTGATTCTTGATGGAAAAAGCTTTGAAGGATTAATAGATACCGG | | | |
| | | GGCCGATGTAACCATTATTAGAGGGCAGGACTGGCCCTCAAACTGGC | | | |
| | | CCCTGTCTGTTTCCTTGACTCACCTTCAAGGAATTGGTTATGCCAGTAA | | | |
| | | CCCAAAACGTAGTTCCAAATTGCTAACCTGGAGAGATGAGGATGGAA | | | |
| | | AATCAGGAAATATTCAGCCGTATGTTATGCAAAATTTGCCTGTAACCC | | | |
| | | TGTGGGGAAGAGATCTGTTGTCACAGATGGGCGTTATCCTGTGCAGT | | | |
| | | TCTAAGGAAATGGTGACTGAACAGACGTTCAGGCAGGGACCCCTGCC | | | |
| | | TGATCGTGGACTAATAAAGAAGGGACAGAAAATTAAGACTTTTGAAG | | | |
| | | ATCTTAAACCCCACTCTAACGTGAGAGGTTTAAAGTATTTTCAGTAGT | | | |
| | | GTAAAATTCCTTTTGTGCTTAAAATTCAGCTGAGAGCAACAGCTCTCA | | | |
| | | AAGCTGTTCTCCAGCTACTCTCTGAGCCAGCTCCCGACAGGAGGCCG | | | |
| | | GAGACTAGCCTCAGCTTTACAATTTGCATTTAAATAAAGTACCTAGAC | | | |
| | | TTCCCCGAAAAAAGTTCTGCTTTTCTACTTTCTCACTGTCTTTCAAGATT | | | |
| | | TTGTCTTTCAAGCAGGTAAATCAACATTCTCGAGGCGGACCAGCGGA | | | |
| | | TGTGCATCCCCGCCCCCCTAGAGCACTCAGGTGGCAGCTGTTATCCCC | | | |
| | | AGTCTCAGGACATTCCAGCATGTGGCCTTCAGTCTGAGTTAAAAATTA | | | |
| | | GGTTTACCCAGAGGACTAGAATAGTAGATATTTCTATATTAATAAAGA | | | |
| | | TTGGTTTTTATTTTGATAGACAGGCTTAGCCCCTTAGCTGACCTCTGGC | | | |
| | | TTTTCACCCTTGCTGTTACTGCAAGGTGTCTTTAGCTCAATAAGGCTGT | | | |
| | | GGAAAAAAACAGGGATGAGGAGGAACGGCTCCCAGCTCCTATTTTA | | | |
| | | GCCACAAATCGTGGTGTTACTAACGACATAATTCTTGCTTAGGCTTTG | | | |
| | | CTAAATCTGAGGTTGATAATTCTCCTTTAGGAGCTGCACAGCGCTCAG | | | |
| | | AACTGTGCATACTGATTTGTGATGGTACAAATTCAGTATGGGCATCGC | | | |
| | | TTGGTGCAGATGGAGGTACTGCAAGGAAAGGTCCCAGCTTGACCATT | | | |
| | | TCTGAGTTTCCTGTGAGATAAACCCGGTTTGAAAGAGGTTGGTACCA | | | |
| | | AATTATATATCCCTCGGCTCTACCTCGCCTCCCCAAAAGGTACCAGAG | | | |
| | | CCACAGGTGTGGATTTTAACAGAATCCACGGGAGGAATCGGGTCCAT | | | |
| | | GTCCACCCAAGCCAAGGTTAAAAGCCCACTCATCTACGGATGAGAAA | | | |
| | | ATCATTTGATCACCTCAGTTAAGCGCTGCCTTATTTTAACTTAATTAAT | | | |
| | | AGGGGGGAGAGAGATTGGAGACTTACTATTGAAAGGGCAAGCCCTT | | | |
| | | CACTGCCTCCCACCCAAATAAAAAAGCCAATTGGCCTTGTACTACAGA | | | |
| | | GCTGGCCGGACCCCTTATCCCTGTTACCCACCAATCATCCAAAAATGC | | | |
| | | GGAGGAATATCAACTTAGTGTTATTCTTATTATAGTGTATTTCACACTT | | | |
| | | GTTCAGTCAAACTTAGCCAGAGTTCCAACGCCCTACTTAAAATTCAAC | | | |
| | | TAGAAAGTTACCTACCAAGTACTAATTAGCATTATAAAGTCAGAGCCT | | | |
| | | ACAGCTCCAGGCTTTTCAGTTAGTTGTTTACTAAGATAAGAAAAGACA | | | |
| | | GTCTTAGCCAGATACAGTTTACCATAATAAAAGTTAAAGAATCCCAGG | | | |
| | | GAAGCAAGTTTTTTCTTTTAGCCCTAGATTCCAGGCAGAACTATTGAG | | | |
| | | CATAGATAATTTTTCCCCCTCAGGCCAGCTTTTTCTTTTTTTTTAATTTT | | | |
| | | GTTAATAAAAGGGAGGAGATGTAGTCTCCCCTCCCCCAGCCTGAAAC | | | |
| | | CTGCTTGCTCAGGGGTGGAGCTTCCCGCTCATCGCTCTGCCACGCCCA | | | |
| | | CTGCTGGAACCTGCGGAGCCACACACGTGCACCTTTCTACTGGACCA | | | |
| | | GAGATTATTCGGCGGGAATCGGGTCCCCTCCCCCTTCCTTCATAACTA | | | |
| | | GTGTCCCAACAATAAAATTTGAGCTTTGATCAGAATGAATTTGTCTTG | | | |
| | | GCTCCGTTTCTTCTTTCGCCCCGTCTAGATTCCTCTCTTACAGCTCGAG | | | |
| | | TGGCCTTCTCAGTCGAACCGTTCACGTTGCGAGCTGCTGGCGGCCGC | | | |
| | | AACAGGGGATCCAGACATGATAAGATACATTGATGAGTTTGGACAAA | | | |
| | | CCACAACTAGAATGCAGTGAAAAAAATGCTTTATTTGTGAAATTTGTG | | | |
| | | ATGCTATTGCTTTATTTGTAACCATTATAAGCTGCAATAAACAAGTTAA | | | |
| | | CAACAACAATTGCATTCATTTTATGTTTCAGGTTCAGGGGGAGGTGTG | | | |
| | | GGAGGTTTTTTCGGATCCTCTAGAGTCGACCTGCAGGCATGCAAGCT | | | |
| | | TGGCGTAATCATGGTCATAGCTGTTTCCTGTGTGAAATTGTTATCCGC | | | |
| | | TCACAATTCCACACAACATACGAGCCGGAAGCATAAAGTGTAAAGCC | | | |
| | | TGGGGTGCCTAATGAGTGAGCTAACTCACATTAATTGCGTTGCGCTCA | | | |
| | | CTGCCCGCTTTCCAGTCGGGAAACCTGTCGTGCCAGCTGCATTAATGA | | | |
| | | ATCGGCCAACGCGCGGGGAGAGGCGGTTTGCGTATTGGGCGCTCTT | | | |
| | | CCGCTTCCTCGCTCACTGACTCGCTGCGCTCGGTCGTTCGGCTGCGGC | | | |
| | | GAGCGGTATCAGCTCACTCAAAGGCGGTAATACGGTTATCCACAGAA | | | |
| | | TCAGGGGATAACGCAGGAAAGAACATGTGAGCAAAAGGCCAGCAAA | | | |
| | | AGGCCAGGAACCGTAAAAAGGCCGCGTTGCTGGCGTTTTTCCATAGG | | | |
| | | CTCCGCCCCCCTGACGAGCATCACAAAAATCGACGCTCAAGTCAGAG | | | |
| | | GTGGCGAAACCCGACAGGACTATAAAGATACCAGGCGTTTCCCCCTG | | | |
| | | GAAGCTCCCTCGTGCGCTCTCCTGTTCCGACCCTGCCGCTTACCGGAT | | | |
| | | ACCTGTCCGCCTTTCTCCCTTCGGGAAGCGTGGCGCTTTCTCATAGCT | | | |
| | | CACGCTGTAGGTATCTCAGTTCGGTGTAGGTCGTTCGCTCCAAGCTG | | | |
| | | GGCTGTGTGCACGAACCCCCCGTTCAGCCCGACCGCTGCGCCTTATCC | | | |
| | | GGTAACTATCGTCTTGAGTCCAACCCGGTAAGACACGACTTATCGCCA | | | |
| | | CTGGCAGCAGCCACTGGTAACAGGATTAGCAGAGCGAGGTATGTAG | | | |
| | | GCGGTGCTACAGAGTTCTTGAAGTGGTGGCCTAACTACGGCTACACT | | | |
| | | AGAAGGACAGTATTTGGTATCTGCGCTCTGCTGAAGCCAGTTACCTTC | | | |
| | | GGAAAAAGAGTTGGTAGCTCTTGATCCGGCAAACAAACCACCGCTGG | | | |
| | | TAGCGGTGGTTTTTTTGTTTGCAAGCAGCAGATTACGCGCAGAAAAA | | | |
| | | AAGGATCTCAAGAAGATCCTTTGATCTTTTCTACGGGGTCTGACGCTC | | | |
| | | AGTGGAACGAAAACTCACGTTAAGGGATTTTGGTCATGAGATTATCA | | | |
| | | AAAAGGATCTTCACCTAGATCCTTTTAAATTAAAAATGAAGTTTTAAA | | | |
| | | TCAATCTAAAGTATATATGAGTAAACTTGGTCTGACAGTTACCAATGC | | | |
| | | TTAATCAGTGAGGCACCTATCTCAGCGATCTGTCTATTTCGTTCATCCA | | | |
| | | TAGTTGCCTGACTCCCCGTCGTGTAGATAACTACGATACGGGAGGGC | | | |
| | | TTACCATCTGGCCCCAGTGCTGCAATGATACCGCGAGACCCACGCTCA | | | |
| | | CCGGCTCCAGATTTATCAGCAATAAACCAGCCAGCCGGAAGGGCCGA | | | |
| | | GCGCAGAAGTGGTCCTGCAACTTTATCCGCCTCCATCCAGTCTATTAA | | | |
| | | TTGTTGCCGGGAAGCTAGAGTAAGTAGTTCGCCAGTTAATAGTTTGC | | | |
| | | GCAACGTTGTTGCCATTGCTACAGGCATCGTGGTGTCACGCTCGTCGT | | | |
| | | TTGGTATGGCTTCATTCAGCTCCGGTTCCCAACGATCAAGGCGAGTTA | | | |
| | | CATGATCCCCCATGTTGTGCAAAAAAGCGGTTAGCTCCTTCGGTCCTC | | | |
| | | CGATCGTTGTCAGAAGTAAGTTGGCCGCAGTGTTATCACTCATGGTTA | | | |
| | | TGGCAGCACTGCATAATTCTCTTACTGTCATGCCATCCGTAAGATGCT | | | |
| | | TTTCTGTGACTGGTGAGTACTCAACCAAGTCATTCTGAGAATAGTGTA | | | |
| | | TGCGGCGACCGAGTTGCTCTTGCCCGGCGTCAATACGGGATAATACC | | | |
| | | GCGCCACATAGCAGAACTTTAAAAGTGCTCATCATTGGAAAACGTTCT | | | |
| | | TCGGGGCGAAAACTCTCAAGGATCTTACCGCTGTTGAGATCCAGTTC | | | |
| | | GATGTAACCCACTCGTGCACCCAACTGATCTTCAGCATCTTTTACTTTC | | | |
| | | ACCAGCGTTTCTGGGTGAGCAAAAACAGGAAGGCAAAATGCCGCAA | | | |
| | | AAAAGGGAATAAGGGCGACACGGAAATGTTGAATACTCATACTCTTC | | | |
| | | CTTTTTCAATATTATTGAAGCATTTATCAGGGTTATTGTCTCATGAGCG | | | |
| | | GATACATATTTGAATGTATTTAGAAAAATAAACAAATAGGGGTTCCG | | | |
| | | CGCACATTTCCCCGAAAAGTGCCACCTGACGTCTAAGAAACCATTATT | | | |
| | | ATCATGACATTAACCTATAAAAATAGGCGTATCACGAGGCCCTTTCGT | | | |
| | | CTCGCGCGTTTCGGTGATGACGGTGAAAACCTCTGACACATGCAGCT | | | |
| | | CCCGGAGACGGTCACAGCTTGTCTGTAAGCGGATGCCGGGAGCAGA | | | |
| | | CAAGCCCGTCAGGGCGCGTCAGCGGGTGTTGGCGGGTGTCGGGGCT | | | |
| | | GGCTTAACTATGCGGCATCAGAGCAGATTGTACTGAGAGTGCACCAT | | | |
| | | ATGCGGTGTGAAATACCGCACAGATGCGTAAGGAGAAAATACCGCA | | | |
| | | TCAGGCGCCATTCGCCATTCAGGCTGCGCAACTGTTGGGAAGGGCGA | | | |
| | | TCGGTGCGGGCCTCTTCGCTATTACGCCAGCTGGCGAAAGGGGGATG | | | |
| | | TGCTGCAAGGCGATTAAGTTGGGTAACGCCAGGGTTTTCCCAGTCAC | | | |
| | | GACGTTGTAAAACGACGGCCAGT | | | |
| DRIVER_ | PLV10051 | GAATTCGAGCTTGCATGCCTGCAGGTCGTTACATAACTTACGGTAAAT | 319 | N/A | |
| pCMV_R- | | GGCCCGCCTGGCTGACCGCCCAACGACCCCCGCCCATTGACGTCAAT | | | |
| U5- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| delPol- | | AAGTGTATCATATGCCAAGTACGCCCCCTATTGACGTCAATGACGGTA | | | |
| LTR_v3 | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTGAGCTTTGATCAGAATGAATTTGTCTTGGCTCCGTTT | | | |
| | | CTTCTTTCGCCCCGTCTAGATTCCTCTCTTACAGCTCGAGTGGCCTTCT | | | |
| | | CAGTCGAACCGTTCACGTTGCGAGCTGCTGGCGGCCGCAACATTTTG | | | |
| | | GCGCCAGAACTGGGACCTGAAGAATGGCAGAGAGATGCTAAGAGGA | | | |
| | | ACGCTGCTTTGGAGCTCCACAGGAAAGGATCTTCGTATCGGACATCG | | | |
| | | GAGCAACGGACAGGTACACATGCTAGCGCTAGCTTAAAATTTCAGTT | | | |
| | | TTGTAAAGTGTTGCTGAGGATGTGGTAGGATACGAATTAAGCTTGAA | | | |
| | | TCAGTGCTAACCCAACGCTGGTTCTGCTTGGGTCAGCAGCGTGTTAAT | | | |
| | | CGGAACTAGAAACGGAAACAGGCAGGTTAGCCGCAGCTTTTTAGGA | | | |
| | | AGCTGCTTAGGTGGAAGAAGAAAGGGTTTAAAGTCATGGATCAGGC | | | |
| | | GGTTGCCCATAGTTTTCAGGAGTTGTTTCAGGCCAGAGGAGTAAGGC | | | |
| | | TTGAAGTACAATTAGTAAAAAATTTTTTAGGTAAGATAGATAGCTGTT | | | |
| | | GCCCATGGTTCAAGGAAGAAGAAACACTAGATTGTGGAACCTGGGA | | | |
| | | GAAAGTTGGTGAGGCCTTAAAAATCACTCAGGCAGATAATTTTACCCT | | | |
| | | AGGCCTCTGGGCACTCATAAATGATGCAATAAAAGATGCCACTTCCCC | | | |
| | | AGGGCTAAGTTGCCCCCAGGCGGAGCTTGTGGTATCTCAGGAGGAG | | | |
| | | TGCCTGTCAGAGAGGGCCTCCTCAGAAAAAGATCTTCTTAACTCAAAA | | | |
| | | ATTGATAAATGTGGAAACTCGGATGAAAAACTGATTTTTAACAAAAA | | | |
| | | TCACTCAGATAGAGGAGCTGCCCATTACCTTAATGAGAATTGGTCCTC | | | |
| | | TTGTGAATCTCCTGCTCAACCTGTAGTCCCCACTTCGGGAGGTGCCAC | | | |
| | | TCATAGGGACACACGACTAAGCGAGTTAGAGTTTGAGATTAAGCTTC | | | |
| | | AGAGGCTGACTAATGAGCTTCGGGAACTAAAAAAGATGTCAGAAGC | | | |
| | | GGAGAAGAGTAACTCTTCTGTAGTTCACCAGGTGCCGCTAGAAAAGG | | | |
| | | TTGTGAGTCAGGCTCATGGGAAAGGACAGAATATCTCTAATACGCTA | | | |
| | | GCCTTTCCTGTGGTTGAGGTAGTTGATCAGCAAGATACTAGGGGCAG | | | |
| | | ACATTACCAGACCTTAGATTTCAAGTTGATAAAAGAGTTAAAGGCGG | | | |
| | | CTGTTGTGCAATATGGCCCTTCAGCCCCATTCACTCAAGCATTACTGG | | | |
| | | ACACAGTTGTGGAGTCACACTTAACCCCTTTAGATTGGAAGACTCTTT | | | |
| | | CTAAGGCTACCCTGTCAGGAGGAGATTTTTTGCTTTGGGATTCTGAAT | | | |
| | | GGCGAGACGCCAGTAAGAAAACTGCTGCTTCTAACGCTCAGGCTGGT | | | |
| | | AATTCAGACTGGGATAGCAACATGCTTTTAGGAGAGGGCCCTTATGA | | | |
| | | GGGACAGACAAATCAGATTGATTTTCCCGTTGCAGTGTACGCGCAAA | | | |
| | | TTGCGACGGCCGCACGCCGTGCTTGGGGAAGGTTGCCAGTCAAAGG | | | |
| | | AGAGATTGGTGGAAGTTTAGCTAGCATTCGGCAGAGTTCTGATGAAC | | | |
| | | CATATCAGGATTTTGTGGACAGGCTATTGATTTCAGCTAGTAGAATCC | | | |
| | | TTGGAAATCCGGACACGGGAAGTCCTTTCGTTATGCAATTGGCTTATG | | | |
| | | AGAATGCTAACGCAATTTGCCGAGCTGCGATTCAACCGCATAAGGGA | | | |
| | | ACGACAGATTTGGCGGGATATGTCCGTCTTTGCGCAGACATCGGGCC | | | |
| | | TTCCTGCGAGACCTTGCAGGGAACCCACGCGCAGGCAATGTTCTCTA | | | |
| | | GGAAACGAGGGAATAGTGCATGCTTTAAATGTGGAAGTTTAGATCAT | | | |
| | | TTTAGAATTGATTGTCCTCAGAACAAGGGCGCCGAGGTTAGACAAAC | | | |
| | | AGGCCGTGCCCCGGGAATATGTCCCCGATGTGGAAAGGGCCGCCACT | | | |
| | | GGGCGAAAGATTGCAAGCATAAAACGAGGGTTTTGAGCCGCCCGGT | | | |
| | | GCCGGGAAACGAGGAAAGGGGTCAGCCCCAGGCCCCAAGTTACTCA | | | |
| | | AAGAAGACAGCTTATGGGGCTCTAAATCTGCTGCCCAGCCAACAAGA | | | |
| | | TCAGTTCTTGAGCTTGTCAGGTCAAACCCAGGAAACGCAAGACTGGA | | | |
| | | CCTCTGTTCCACTGTCCATGCAGCATTAACCCCAGAAGTGGGAGTCCA | | | |
| | | AACTCTGCCTACCGGAGTCTTTGGACCACTACCTGTAGGAACCTGTGG | | | |
| | | TTTTCTCTTAGGACGAAGCAGTTCTATTGTAGAAGGCCTGCAGATTTA | | | |
| | | TCCAGGTGTTATAAGTAATGATTATGAGGGAGAAATTAAAATCATAG | | | |
| | | CCGCTTGCCCTCGTGGTGCTATAACTATACCCGCTAATCAGAAAATTG | | | |
| | | CTCAACTTACCTTGATCCCCTTGCGCTGGTCACTATCTAAATTCTCTAA | | | |
| | | AAATGAAGAAGGACAGATTAACTTTGACTCCTCTGGCGTAAATTGGG | | | |
| | | TGAAATCTATCACTAATCAGAGACCTAACCTTAAATTGATTCTTGATG | | | |
| | | GAAAAAGCTTTGAAGGATTAATAGATACCGGGGCCGATGTAACCATT | | | |
| | | ATTAGAGGGCAGGACTGGCCCTCAAACTGGCCCCTGTCTGTTTCCTTG | | | |
| | | ACTCACCTTCAAGGAATTGGTTATGCCAGTAACCCAAAACGTAGTTCC | | | |
| | | AAATTGCTAACCTGGAGAGATGAGGATGGAAAATCAGGAAATATTCA | | | |
| | | GCCGTATGTTATGCAAAATTTGCCTGTAACCCTGTGGGGAAGAGATC | | | |
| | | TGTTGTCACAGATGGGCGTTATCCTGTGCAGTTCTAAGGAAATGGTG | | | |
| | | ACTGAACAGACGTTCAGGCAGGGACCCCTGCCTGATCGTGGACTAAT | | | |
| | | AAAGAAGGGACAGAAAATTAAGACTTTTGAAGATCTTAAACCCCACT | | | |
| | | CTAACGTGAGAGGTTTAAAGTATTTTCAGTAGTGTAAAATTCCTTTTG | | | |
| | | TGCTTAAAATTCAGCTGAGAGCAACAGCTCTCAAAGCTGTTCTCCAGC | | | |
| | | TACTCTCTGAGCCAGCTCCCGACAGGAGGCCGGAGACTAGCCTCAGC | | | |
| | | TTTACAATTTGCATTTAAATAAAGTACCTAGACTTCCCCGAAAAAAGT | | | |
| | | TCTGCTTTTCTACTTTCTCACTGTCTTTCAAGATTTTGTCTTTCAAGCAG | | | |
| | | GTAAATCAACATTCTCGAGGCGGACCAGCGGATGTGCATCCCCGCCC | | | |
| | | CCCTAGAGCACTCAGGTGGCAGCTGTTATCCCCAGTCTCAGGACATTC | | | |
| | | CAGCATGTGGCCTTCAGTCTGAGTTAAAAATTAGGTTTACCCAGAGG | | | |
| | | ACTAGAATAGTAGATATTTCTATATTAATAAAGATTGGTTTTTATTTTG | | | |
| | | ATAGACAGGCTTAGCCCCTTAGCTGACCTCTGGCTTTTCACCCTTGCT | | | |
| | | GTTACTGCAAGGTGTCTTTAGCTCAATAAGGCTGTGGAAAAAAACAG | | | |
| | | GGATGAGGAGGAACGGCTCCCAGCTCCTATTTTAGCCACAAATCGTG | | | |
| | | GTGTTACTAACGACATAATTCTTGCTTAGGCTTTGCTAAATCTGAGGT | | | |
| | | TGATAATTCTCCTTTAGGAGCTGCACAGCGCTCAGAACTGTGCATACT | | | |
| | | GATTTGTGATGGTACAAATTCAGTATGGGCATCGCTTGGTGCAGATG | | | |
| | | GAGGTACTGCAAGGAAAGGTCCCAGCTTGACCATTTCTGAGTTTCCT | | | |
| | | GTGAGATAAACCCGGTTTGAAAGAGGTTGGTACCAAATTATATATCC | | | |
| | | CTCGGCTCTACCTCGCCTCCCCAAAAGGTACCAGAGCCACAGGTGTG | | | |
| | | GATTTTAACAGAATCCACGGGAGGAATCGGGTCCATGTCCACCCAAG | | | |
| | | CCAAGGTTAAAAGCCCACTCATCTACGGATGAGAAAATCATTTGATCA | | | |
| | | CCTCAGTTAAGCGCTGCCTTATTTTAACTTAATTAATAGGGGGGAGAG | | | |
| | | AGATTGGAGACTTACTATTGAAAGGGCAAGCCCTTCACTGCCTCCCAC | | | |
| | | CCAAATAAAAAAGCCAATTGGCCTTGTACTACAGAGCTGGCCGGACC | | | |
| | | CCTTATCCCTGTTACCCACCAATCATCCAAAAATGCGGAGGAATATCA | | | |
| | | ACTTAGTGTTATTCTTATTATAGTGTATTTCACACTTGTTCAGTCAAAC | | | |
| | | TTAGCCAGAGTTCCAACGCCCTACTTAAAATTCAACTAGAAAGTTACC | | | |
| | | TACCAAGTACTAATTAGCATTATAAAGTCAGAGCCTACAGCTCCAGGC | | | |
| | | TTTTCAGTTAGTTGTTTACTAAGATAAGAAAAGACAGTCTTAGCCAGA | | | |
| | | TACAGTTTACCATAATAAAAGTTAAAGAATCCCAGGGAAGCAAGTTTT | | | |
| | | TTCTTTTAGCCCTAGATTCCAGGCAGAACTATTGAGCATAGATAATTTT | | | |
| | | TCCCCCTCAGGCCAGCTTTTTCTTTTTTTTTAATTTTGTTAATAAAAGG | | | |
| | | GAGGAGATGTAGTCTCCCCTCCCCCAGCCTGAAACCTGCTTGCTCAGG | | | |
| | | GGTGGAGCTTCCCGCTCATCGCTCTGCCACGCCCACTGCTGGAACCTG | | | |
| | | CGGAGCCACACACGTGCACCTTTCTACTGGACCAGAGATTATTCGGC | | | |
| | | GGGAATCGGGTCCCCTCCCCCTTCCTTCATAACTAGTGTCCCAACAAT | | | |
| | | AAAATTTGAGCTTTGATCAGAATGAATTTGTCTTGGCTCCGTTTCTTCT | | | |
| | | TTCGCCCCGTCTAGATTCCTCTCTTACAGCTCGAGTGGCCTTCTCAGTC | | | |
| | | GAACCGTTCACGTTGCGAGCTGCTGGCGGCCGCAACAGGGGATCCA | | | |
| | | GACATGATAAGATACATTGATGAGTTTGGACAAACCACAACTAGAAT | | | |
| | | GCAGTGAAAAAAATGCTTTATTTGTGAAATTTGTGATGCTATTGCTTT | | | |
| | | ATTTGTAACCATTATAAGCTGCAATAAACAAGTTAACAACAACAATTG | | | |
| | | CATTCATTTTATGTTTCAGGTTCAGGGGGAGGTGTGGGAGGTTTTTTC | | | |
| | | GGATCCTCTAGAGTCGACCTGCAGGCATGCAAGCTTGGCGTAATCAT | | | |
| | | GGTCATAGCTGTTTCCTGTGTGAAATTGTTATCCGCTCACAATTCCAC | | | |
| | | ACAACATACGAGCCGGAAGCATAAAGTGTAAAGCCTGGGGTGCCTA | | | |
| | | ATGAGTGAGCTAACTCACATTAATTGCGTTGCGCTCACTGCCCGCTTT | | | |
| | | CCAGTCGGGAAACCTGTCGTGCCAGCTGCATTAATGAATCGGCCAAC | | | |
| | | GCGCGGGGAGAGGCGGTTTGCGTATTGGGCGCTCTTCCGCTTCCTCG | | | |
| | | CTCACTGACTCGCTGCGCTCGGTCGTTCGGCTGCGGCGAGCGGTATC | | | |
| | | AGCTCACTCAAAGGCGGTAATACGGTTATCCACAGAATCAGGGGATA | | | |
| | | ACGCAGGAAAGAACATGTGAGCAAAAGGCCAGCAAAAGGCCAGGA | | | |
| | | ACCGTAAAAAGGCCGCGTTGCTGGCGTTTTTCCATAGGCTCCGCCCCC | | | |
| | | CTGACGAGCATCACAAAAATCGACGCTCAAGTCAGAGGTGGCGAAA | | | |
| | | CCCGACAGGACTATAAAGATACCAGGCGTTTCCCCCTGGAAGCTCCCT | | | |
| | | CGTGCGCTCTCCTGTTCCGACCCTGCCGCTTACCGGATACCTGTCCGC | | | |
| | | CTTTCTCCCTTCGGGAAGCGTGGCGCTTTCTCATAGCTCACGCTGTAG | | | |
| | | GTATCTCAGTTCGGTGTAGGTCGTTCGCTCCAAGCTGGGCTGTGTGC | | | |
| | | ACGAACCCCCCGTTCAGCCCGACCGCTGCGCCTTATCCGGTAACTATC | | | |
| | | GTCTTGAGTCCAACCCGGTAAGACACGACTTATCGCCACTGGCAGCA | | | |
| | | GCCACTGGTAACAGGATTAGCAGAGCGAGGTATGTAGGCGGTGCTA | | | |
| | | CAGAGTTCTTGAAGTGGTGGCCTAACTACGGCTACACTAGAAGGACA | | | |
| | | GTATTTGGTATCTGCGCTCTGCTGAAGCCAGTTACCTTCGGAAAAAGA | | | |
| | | GTTGGTAGCTCTTGATCCGGCAAACAAACCACCGCTGGTAGCGGTGG | | | |
| | | TTTTTTTGTTTGCAAGCAGCAGATTACGCGCAGAAAAAAAGGATCTCA | | | |
| | | AGAAGATCCTTTGATCTTTTCTACGGGGTCTGACGCTCAGTGGAACGA | | | |
| | | AAACTCACGTTAAGGGATTTTGGTCATGAGATTATCAAAAAGGATCTT | | | |
| | | CACCTAGATCCTTTTAAATTAAAAATGAAGTTTTAAATCAATCTAAAGT | | | |
| | | ATATATGAGTAAACTTGGTCTGACAGTTACCAATGCTTAATCAGTGAG | | | |
| | | GCACCTATCTCAGCGATCTGTCTATTTCGTTCATCCATAGTTGCCTGAC | | | |
| | | TCCCCGTCGTGTAGATAACTACGATACGGGAGGGCTTACCATCTGGC | | | |
| | | CCCAGTGCTGCAATGATACCGCGAGACCCACGCTCACCGGCTCCAGA | | | |
| | | TTTATCAGCAATAAACCAGCCAGCCGGAAGGGCCGAGCGCAGAAGT | | | |
| | | GGTCCTGCAACTTTATCCGCCTCCATCCAGTCTATTAATTGTTGCCGG | | | |
| | | GAAGCTAGAGTAAGTAGTTCGCCAGTTAATAGTTTGCGCAACGTTGT | | | |
| | | TGCCATTGCTACAGGCATCGTGGTGTCACGCTCGTCGTTTGGTATGGC | | | |
| | | TTCATTCAGCTCCGGTTCCCAACGATCAAGGCGAGTTACATGATCCCC | | | |
| | | CATGTTGTGCAAAAAAGCGGTTAGCTCCTTCGGTCCTCCGATCGTTGT | | | |
| | | CAGAAGTAAGTTGGCCGCAGTGTTATCACTCATGGTTATGGCAGCAC | | | |
| | | TGCATAATTCTCTTACTGTCATGCCATCCGTAAGATGCTTTTCTGTGAC | | | |
| | | TGGTGAGTACTCAACCAAGTCATTCTGAGAATAGTGTATGCGGCGAC | | | |
| | | CGAGTTGCTCTTGCCCGGCGTCAATACGGGATAATACCGCGCCACAT | | | |
| | | AGCAGAACTTTAAAAGTGCTCATCATTGGAAAACGTTCTTCGGGGCG | | | |
| | | AAAACTCTCAAGGATCTTACCGCTGTTGAGATCCAGTTCGATGTAACC | | | |
| | | CACTCGTGCACCCAACTGATCTTCAGCATCTTTTACTTTCACCAGCGTT | | | |
| | | TCTGGGTGAGCAAAAACAGGAAGGCAAAATGCCGCAAAAAAGGGAA | | | |
| | | TAAGGGCGACACGGAAATGTTGAATACTCATACTCTTCCTTTTTCAAT | | | |
| | | ATTATTGAAGCATTTATCAGGGTTATTGTCTCATGAGCGGATACATAT | | | |
| | | TTGAATGTATTTAGAAAAATAAACAAATAGGGGTTCCGCGCACATTTC | | | |
| | | CCCGAAAAGTGCCACCTGACGTCTAAGAAACCATTATTATCATGACAT | | | |
| | | TAACCTATAAAAATAGGCGTATCACGAGGCCCTTTCGTCTCGCGCGTT | | | |
| | | TCGGTGATGACGGTGAAAACCTCTGACACATGCAGCTCCCGGAGACG | | | |
| | | GTCACAGCTTGTCTGTAAGCGGATGCCGGGAGCAGACAAGCCCGTCA | | | |
| | | GGGCGCGTCAGCGGGTGTTGGCGGGTGTCGGGGCTGGCTTAACTAT | | | |
| | | GCGGCATCAGAGCAGATTGTACTGAGAGTGCACCATATGCGGTGTGA | | | |
| | | AATACCGCACAGATGCGTAAGGAGAAAATACCGCATCAGGCGCCATT | | | |
| | | CGCCATTCAGGCTGCGCAACTGTTGGGAAGGGCGATCGGTGCGGGC | | | |
| | | CTCTTCGCTATTACGCCAGCTGGCGAAAGGGGGATGTGCTGCAAGGC | | | |
| | | GATTAAGTTGGGTAACGCCAGGGTTTTCCCAGTCACGACGTTGTAAA | | | |
| | | ACGACGGCCAGT | | | |
| DRIVER_ | PLV10052 | GAATTCGAGCTTGCATGCCTGCAGGTCGTTACATAACTTACGGTAAAT | 320 | N/A | |
| pCMV_R- | | GGCCCGCCTGGCTGACCGCCCAACGACCCCCGCCCATTGACGTCAAT | | | |
| U5-PBS*- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| LTR_v1 | | AAGTGTATCATATGCCAAGTACGCCCCCTATTGACGTCAATGACGGTA | | | |
| | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGAGCTTTGATCAGAATGAA | | | |
| | | TTTGTCTTGGCTCCGTTTCTTCTTTCGCCCCGTCTAGATTCCTCTCTTAC | | | |
| | | AGCTCGAGTGGCCTTCTCAGTCGAACCGTTCACGTTGCGAGCTGCTG | | | |
| | | GCGGCCGCAACATTTTAGCGACTAAACACATCACTGAAGAATCCGAG | | | |
| | | AGAGATGCTAAGAGGAACGCTGCTTTGGAGCTCCACAGGAAAGGAT | | | |
| | | CTTCGTATCGGACATCGGAGCAACGGACAGGTACACATGCTAGCGCT | | | |
| | | AGCTTAAAATTTCAGTTTTGTAAAGTGTTGCTGAGGATGTGGTAGGA | | | |
| | | TACGAATTAAGCTTGAATCAGTGCTAACCCAACGCTGGTTCTGCTTGG | | | |
| | | GTCAGCAGCGTGTTAATCGGAACTAGAAACGGAAACAGGCAGGTTA | | | |
| | | GCCGCAGCTTTTTAGGAAGCTGCTTAGGTGGAAGAAGAAAGGGTTTA | | | |
| | | AAGTCATGGATCAGGCGGTTGCCCATAGTTTTCAGGAGTTGTTTCAG | | | |
| | | GCCAGAGGAGTAAGGCTTGAAGTACAATTAGTAAAAAATTTTTTAGG | | | |
| | | TAAGATAGATAGCTGTTGCCCATGGTTCAAGGAAGAAGAAACACTAG | | | |
| | | ATTGTGGAACCTGGGAGAAAGTTGGTGAGGCCTTAAAAATCACTCAG | | | |
| | | GCAGATAATTTTACCCTAGGCCTCTGGGCACTCATAAATGATGCAATA | | | |
| | | AAAGATGCCACTTCCCCAGGGCTAAGTTGCCCCCAGGCGGAGCTTGT | | | |
| | | GGTATCTCAGGAGGAGTGCCTGTCAGAGAGGGCCTCCTCAGAAAAA | | | |
| | | GATCTTCTTAACTCAAAAATTGATAAATGTGGAAACTCGGATGAAAA | | | |
| | | ACTGATTTTTAACAAAAATCACTCAGATAGAGGAGCTGCCCATTACCT | | | |
| | | TAATGAGAATTGGTCCTCTTGTGAATCTCCTGCTCAACCTGTAGTCCC | | | |
| | | CACTTCGGGAGGTGCCACTCATAGGGACACACGACTAAGCGAGTTAG | | | |
| | | AGTTTGAGATTAAGCTTCAGAGGCTGACTAATGAGCTTCGGGAACTA | | | |
| | | AAAAAGATGTCAGAAGCGGAGAAGAGTAACTCTTCTGTAGTTCACCA | | | |
| | | GGTGCCGCTAGAAAAGGTTGTGAGTCAGGCTCATGGGAAAGGACAG | | | |
| | | AATATCTCTAATACGCTAGCCTTTCCTGTGGTTGAGGTAGTTGATCAG | | | |
| | | CAAGATACTAGGGGCAGACATTACCAGACCTTAGATTTCAAGTTGAT | | | |
| | | AAAAGAGTTAAAGGCGGCTGTTGTGCAATATGGCCCTTCAGCCCCAT | | | |
| | | TCACTCAAGCATTACTGGACACAGTTGTGGAGTCACACTTAACCCCTT | | | |
| | | TAGATTGGAAGACTCTTTCTAAGGCTACCCTGTCAGGAGGAGATTTTT | | | |
| | | TGCTTTGGGATTCTGAATGGCGAGACGCCAGTAAGAAAACTGCTGCT | | | |
| | | TCTAACGCTCAGGCTGGTAATTCAGACTGGGATAGCAACATGCTTTTA | | | |
| | | GGAGAGGGCCCTTATGAGGGACAGACAAATCAGATTGATTTTCCCGT | | | |
| | | TGCAGTGTACGCGCAAATTGCGACGGCCGCACGCCGTGCTTGGGGA | | | |
| | | AGGTTGCCAGTCAAAGGAGAGATTGGTGGAAGTTTAGCTAGCATTCG | | | |
| | | GCAGAGTTCTGATGAACCATATCAGGATTTTGTGGACAGGCTATTGA | | | |
| | | TTTCAGCTAGTAGAATCCTTGGAAATCCGGACACGGGAAGTCCTTTCG | | | |
| | | TTATGCAATTGGCTTATGAGAATGCTAACGCAATTTGCCGAGCTGCGA | | | |
| | | TTCAACCGCATAAGGGAACGACAGATTTGGCGGGATATGTCCGTCTT | | | |
| | | TGCGCAGACATCGGGCCTTCCTGCGAGACCTTGCAGGGAACCCACGC | | | |
| | | GCAGGCAATGTTCTCTAGGAAACGAGGGAATAGTGCATGCTTTAAAT | | | |
| | | GTGGAAGTTTAGATCATTTTAGAATTGATTGTCCTCAGAACAAGGGC | | | |
| | | GCCGAGGTTAGACAAACAGGCCGTGCCCCGGGAATATGTCCCCGATG | | | |
| | | TGGAAAGGGCCGCCACTGGGCGAAAGATTGCAAGCATAAAACGAGG | | | |
| | | GTTTTGAGCCGCCCGGTGCCGGGAAACGAGGAAAGGGGTCAGCCCC | | | |
| | | AGGCCCCAAGTTACTCAAAGAAGACAGCTTATGGGGCTCTAAATCTG | | | |
| | | CTGCCCAGCCAACAAGATCAGTTCTTGAGCTTGTCAGGTCAAACCCAG | | | |
| | | GAAACGCAAGACTGGACCTCTGTTCCACTGTCCATGCAGCATTAACCC | | | |
| | | CAGAAGTGGGAGTCCAAACTCTGCCTACCGGAGTCTTTGGACCACTA | | | |
| | | CCTGTAGGAACCTGTGGTTTTCTCTTAGGACGAAGCAGTTCTATTGTA | | | |
| | | GAAGGCCTGCAGATTTATCCAGGTGTTATAAGTAATGATTATGAGGG | | | |
| | | AGAAATTAAAATCATAGCCGCTTGCCCTCGTGGTGCTATAACTATACC | | | |
| | | CGCTAATCAGAAAATTGCTCAACTTACCTTGATCCCCTTGCGCTGGTC | | | |
| | | ACTATCTAAATTCTCTAAAAATGAAGAAGGACAGATTAACTTTGACTC | | | |
| | | CTCTGGCGTAAATTGGGTGAAATCTATCACTAATCAGAGACCTAACCT | | | |
| | | TAAATTGATTCTTGATGGAAAAAGCTTTGAAGGATTAATAGATACCG | | | |
| | | GGGCCGATGTAACCATTATTAGAGGGCAGGACTGGCCCTCAAACTGG | | | |
| | | CCCCTGTCTGTTTCCTTGACTCACCTTCAAGGAATTGGTTATGCCAGTA | | | |
| | | ACCCAAAACGTAGTTCCAAATTGCTAACCTGGAGAGATGAGGATGGA | | | |
| | | AAATCAGGAAATATTCAGCCGTATGTTATGCAAAATTTGCCTGTAACC | | | |
| | | CTGTGGGGAAGAGATCTGTTGTCACAGATGGGCGTTATCCTGTGCAG | | | |
| | | TTCTAAGGAAATGGTGACTGAACAGACGTTCAGGCAGGGACCCCTGC | | | |
| | | CTGATCGTGGACTAATAAAGAAGGGACAGAAAATTAAGACTTTTGAA | | | |
| | | GATCTTAAACCCCACTCTAACGTGAGAGGTTTAAAGTATTTTCAGTAG | | | |
| | | TGGCCGCTGTCTTGCCTGCATCCCACGCCGAAAAAATTCAATGGCGTA | | | |
| | | ATGATATTCCGGTGTGGGTAGATCAGTGGTCTTTACCTAAAGAGAAA | | | |
| | | ATAGAGGCCGCTTCTCTGCTAGTGCAGGAGCAGTTAGAAGCAGGACA | | | |
| | | TTTGGTGGAGTCTCATTCTCCCTGGAATACACCCATTTTCATTATCAGG | | | |
| | | AAGAAATCGGGAAAATGGAGACTGTTGCAAGATTTAAGAAAGGTTA | | | |
| | | ATGAAACCATGGTACTTATGGGAACTTTACAACCGGGGCTCCCCTCCC | | | |
| | | CAGTAGCCATTCCTAAGGGATACTATAAGATTGTTATAGATTTGAAAG | | | |
| | | ATTGTTTCTTTACCATCCCTTTGCATCCAGAGGATTGTGAGAGATTTGC | | | |
| | | TTTTAGTGTTCCTTCTGTAAATTTCAAGGAACCCATGAAAAGATATCA | | | |
| | | ATGGACAGTTCTCCCGCAGGGGATGGCTAATAGTCCCACCTTATGTCA | | | |
| | | AAAGTTTGTGGCAAAGGCAATTCAGCCTGTTAGACAACAATGGCCAA | | | |
| | | ATATTTACATCATTCATTTCACAGATGATGTTTTGATGGCGGGAAAGG | | | |
| | | ACCCCCAAGATTTGCTTTTGTGTTATGGAGACTTACGAAAGGCCCTGG | | | |
| | | CTGATAAGGGATTACAAATTGCTTCTGAAAAGATACAAACTCAGGAT | | | |
| | | CCTTATAATTATTTGGGTTTTAGACTCACTGACCAAGCTGTTTTTCACC | | | |
| | | AGAAAATTGTTATTCGTAGAGATAACTTAAGGACCTTAAATGATTTTC | | | |
| | | AAAAATTGTTAGGTGATATAAACTGGCTTCGCCCCTATCTAAAGCTTA | | | |
| | | CTACAGGGGAGTTGAAACCTTTATTTGATATTCTTAAAGGGAGTTCTG | | | |
| | | ATCCTACTTCCCCTAGATCCCTAACCTCAGAAGGTTTACTGGCCTTACA | | | |
| | | GCTAGTGGAAAAGGCTATTGAAGAACAGTTTGTCACTTACATAGATT | | | |
| | | ACTCCCTGCCGCTGCACCTGTTAATTTTTAACACGACTCATGTGCCTAC | | | |
| | | GGGATTGCTATGGCAAAAATTTCCTATAATGTGGATACATTCAAGGAT | | | |
| | | TTCTCCCAAACGTAATATTTTGCCATATCATGAAGCAGTGGCTCAGAT | | | |
| | | GATTATCACTGGAAGAAGGCAGGCATTGACTTATTTTGGAAAGGAGC | | | |
| | | CAGATATCATTGTCCAGCCTTACAGCGTGAGTCAGGACACTTGGCTG | | | |
| | | AAACAGCATAGTACAGATTGGTTGCTTGCACAATTAGGGTTTGAAGG | | | |
| | | AACTATAGATAGCCACTACCCCCAAGATAGGTTGATAAAATTCTTAAA | | | |
| | | TGTACATGATATGATATTTCCTAAGATGACTTCCTTACAGCCTTTAAAT | | | |
| | | AATGCTCTATTGATTTTTACTGATGGCTCCTCTAAAGGGCGAGCTGGA | | | |
| | | TATCTTATTAGTAATCAACAGGTTATCGTAGAGACTCCTGGTCTCTCG | | | |
| | | GCTCAGCTCGCCGAACTAACAGCAGTACTGAAGGTTTTTCAGTCTGTA | | | |
| | | CAGGAGGCTTTTAATATTTTTACTGACAGTTTATATGTTGCTCAGTCA | | | |
| | | GTACCCTTATTGGAAACCTGTGGTACTTTTAACTTCAATACGCCGTCA | | | |
| | | GGATCTTTATTTTCAGAATTACAAAACATCATTCTCGCCCGGAAAAAT | | | |
| | | CCGTTTTATATTGGCCACATACGGTCTCACTCTGGTCTTCCTGGACCTC | | | |
| | | TGGCAGAGGGTAATAATTGCATTGACAGAGCTCTAATAGGAGAAGCC | | | |
| | | TTAGTTTCAGATCGGGTTGCTTTGGCCCAACGTGATCATGAAAGGTTT | | | |
| | | CATCTCTCTAGCCATACCCTAAGGCTCCGACATAAGATCACCAAGGAG | | | |
| | | CAAGCGAGAATGATTGTAAAACAATGTCCTAAATGTATTACTTTATCT | | | |
| | | CCAGTGCCGCATCTAGGAGTTAATCCTAGAGGCCTTATGCCTAATCAT | | | |
| | | ATTTGGCAAATGGATATAACCCATTATGCAGAATTTGGAAAACTAAA | | | |
| | | ATATATACATGTTTGCATTGATACTTGTTCAGGATTTCTCTTTGCTTCTC | | | |
| | | TGCATACAGGAGAAGCTTCAAAAAACGTAATTGATCATTGCCTACAA | | | |
| | | GCATTTAATGCCATGGGATTACCTAAACTTATTAAGACAGACAATGG | | | |
| | | GCCATCTTATTCCAGTAAAAACTTTATTTCATTCTGTAAAGAATTCGGT | | | |
| | | ATTAAACATAAAACTGGAATTCCTTACAACCCCATGGGACAAGGAAT | | | |
| | | AGTTGAACGTGCTCATCGCACCTTAAAGAATTGGCTCTTTAAGACAAA | | | |
| | | AGAGGGGCAGCTATATCCCCCAAGGTCTCCAAAGGCCCACCTTGCCT | | | |
| | | TCACCTTATTTGTCCTAAATTTCTTGCACACCGATATCAAGGGCCAGTC | | | |
| | | TGCAGCGGATCGCCACTGGCATCCAGTTACTTCTAATTCTTATGCATT | | | |
| | | GGTAAAATGGAAGGACCCCCTGACTAATGAATGGAAGGGTCCAGAT | | | |
| | | CCAGTTCTAATTTGGGGTAGAGGCTCAGTTTGTGTTTTTTCACGAGAT | | | |
| | | GAAGATGGAGCACGGTGGCTGCCAGAGAGATTAATTCGTCAGACGA | | | |
| | | ACACAGATTCTGACTCTTCTGGTAAGTATCATTCTAAAGACTAAAATT | | | |
| | | CCTTTTGTGCTTAAAATTCAGCTGAGAGCAACAGCTCTCAAAGCTGTT | | | |
| | | CTCCAGCTACTCTCTGAGCCAGCTCCCGACAGGAGGCCGGAGACTAG | | | |
| | | CCTCAGCTTTACAATTTGCATTTAAATAAAGTACCTAGACTTCCCCGAA | | | |
| | | AAAAGTTCTGCTTTTCTACTTTCTCACTGTCTTTCAAGATTTTGTCTTTC | | | |
| | | AAGCAGGTAAATCAACATTCTCGAGGCGGACCAGCGGATGTGCATCC | | | |
| | | CCGCCCCCCTAGAGCACTCAGGTGGCAGCTGTTATCCCCAGTCTCAGG | | | |
| | | ACATTCCAGCATGTGGCCTTCAGTCTGAGTTAAAAATTAGGTTTACCC | | | |
| | | AGAGGACTAGAATAGTAGATATTTCTATATTAATAAAGATTGGTTTTT | | | |
| | | ATTTTGATAGACAGGCTTAGCCCCTTAGCTGACCTCTGGCTTTTCACCC | | | |
| | | TTGCTGTTACTGCAAGGTGTCTTTAGCTCAATAAGGCTGTGGAAAAAA | | | |
| | | ACAGGGATGAGGAGGAACGGCTCCCAGCTCCTATTTTAGCCACAAAT | | | |
| | | CGTGGTGTTACTAACGACATAATTCTTGCTTAGGCTTTGCTAAATCTG | | | |
| | | AGGTTGATAATTCTCCTTTAGGAGCTGCACAGCGCTCAGAACTGTGCA | | | |
| | | TACTGATTTGTGATGGTACAAATTCAGTATGGGCATCGCTTGGTGCA | | | |
| | | GATGGAGGTACTGCAAGGAAAGGTCCCAGCTTGACCATTTCTGAGTT | | | |
| | | TCCTGTGAGATAAACCCGGTTTGAAAGAGGTTGGTACCAAATTATAT | | | |
| | | ATCCCTCGGCTCTACCTCGCCTCCCCAAAAGGTACCAGAGCCACAGGT | | | |
| | | GTGGATTTTAACAGAATCCACGGGAGGAATCGGGTCCATGTCCACCC | | | |
| | | AAGCCAAGGTTAAAAGCCCACTCATCTACGGATGAGAAAATCATTTG | | | |
| | | ATCACCTCAGTTAAGCGCTGCCTTATTTTAACTTAATTAATAGGGGGG | | | |
| | | AGAGAGATTGGAGACTTACTATTGAAAGGGCAAGCCCTTCACTGCCT | | | |
| | | CCCACCCAAATAAAAAAGCCAATTGGCCTTGTACTACAGAGCTGGCC | | | |
| | | GGACCCCTTATCCCTGTTACCCACCAATCATCCAAAAATGCGGAGGAA | | | |
| | | TATCAACTTAGTGTTATTCTTATTATAGTGTATTTCACACTTGTTCAGTC | | | |
| | | AAACTTAGCCAGAGTTCCAACGCCCTACTTAAAATTCAACTAGAAAGT | | | |
| | | TACCTACCAAGTACTAATTAGCATTATAAAGTCAGAGCCTACAGCTCC | | | |
| | | AGGCTTTTCAGTTAGTTGTTTACTAAGATAAGAAAAGACAGTCTTAGC | | | |
| | | CAGATACAGTTTACCATAATAAAAGTTAAAGAATCCCAGGGAAGCAA | | | |
| | | GTTTTTTCTTTTAGCCCTAGATTCCAGGCAGAACTATTGAGCATAGAT | | | |
| | | AATTTTTCCCCCTCAGGCCAGCTTTTTCTTTTTTTTTAATTTTGTTAATA | | | |
| | | AAAGGGAGGAGATGTAGTCTCCCCTCCCCCAGCCTGAAACCTGCTTG | | | |
| | | CTCAGGGGTGGAGCTTCCCGCTCATCGCTCTGCCACGCCCACTGCTG | | | |
| | | GAACCTGCGGAGCCACACACGTGCACCTTTCTACTGGACCAGAGATT | | | |
| | | ATTCGGCGGGAATCGGGTCCCCTCCCCCTTCCTTCATAACTAGTGTCC | | | |
| | | CAACAATAAAATTTGAGCTTTGATCAGAATGAATTTGTCTTGGCTCCG | | | |
| | | TTTCTTCTTTCGCCCCGTCTAGATTCCTCTCTTACAGCTCGAGTGGCCT | | | |
| | | TCTCAGTCGAACCGTTCACGTTGCGAGCTGCTGGCGGCCGCAACAGG | | | |
| | | GGATCCAGACATGATAAGATACATTGATGAGTTTGGACAAACCACAA | | | |
| | | CTAGAATGCAGTGAAAAAAATGCTTTATTTGTGAAATTTGTGATGCTA | | | |
| | | TTGCTTTATTTGTAACCATTATAAGCTGCAATAAACAAGTTAACAACA | | | |
| | | ACAATTGCATTCATTTTATGTTTCAGGTTCAGGGGGAGGTGTGGGAG | | | |
| | | GTTTTTTCGGATCCTCTAGAGTCGACCTGCAGGCATGCAAGCTTGGCG | | | |
| | | TAATCATGGTCATAGCTGTTTCCTGTGTGAAATTGTTATCCGCTCACAA | | | |
| | | TTCCACACAACATACGAGCCGGAAGCATAAAGTGTAAAGCCTGGGGT | | | |
| | | GCCTAATGAGTGAGCTAACTCACATTAATTGCGTTGCGCTCACTGCCC | | | |
| | | GCTTTCCAGTCGGGAAACCTGTCGTGCCAGCTGCATTAATGAATCGG | | | |
| | | CCAACGCGCGGGGAGAGGCGGTTTGCGTATTGGGCGCTCTTCCGCTT | | | |
| | | CCTCGCTCACTGACTCGCTGCGCTCGGTCGTTCGGCTGCGGCGAGCG | | | |
| | | GTATCAGCTCACTCAAAGGCGGTAATACGGTTATCCACAGAATCAGG | | | |
| | | GGATAACGCAGGAAAGAACATGTGAGCAAAAGGCCAGCAAAAGGCC | | | |
| | | AGGAACCGTAAAAAGGCCGCGTTGCTGGCGTTTTTCCATAGGCTCCG | | | |
| | | CCCCCCTGACGAGCATCACAAAAATCGACGCTCAAGTCAGAGGTGGC | | | |
| | | GAAACCCGACAGGACTATAAAGATACCAGGCGTTTCCCCCTGGAAGC | | | |
| | | TCCCTCGTGCGCTCTCCTGTTCCGACCCTGCCGCTTACCGGATACCTGT | | | |
| | | CCGCCTTTCTCCCTTCGGGAAGCGTGGCGCTTTCTCATAGCTCACGCT | | | |
| | | GTAGGTATCTCAGTTCGGTGTAGGTCGTTCGCTCCAAGCTGGGCTGT | | | |
| | | GTGCACGAACCCCCCGTTCAGCCCGACCGCTGCGCCTTATCCGGTAAC | | | |
| | | TATCGTCTTGAGTCCAACCCGGTAAGACACGACTTATCGCCACTGGCA | | | |
| | | GCAGCCACTGGTAACAGGATTAGCAGAGCGAGGTATGTAGGCGGTG | | | |
| | | CTACAGAGTTCTTGAAGTGGTGGCCTAACTACGGCTACACTAGAAGG | | | |
| | | ACAGTATTTGGTATCTGCGCTCTGCTGAAGCCAGTTACCTTCGGAAAA | | | |
| | | AGAGTTGGTAGCTCTTGATCCGGCAAACAAACCACCGCTGGTAGCGG | | | |
| | | TGGTTTTTTTGTTTGCAAGCAGCAGATTACGCGCAGAAAAAAAGGAT | | | |
| | | CTCAAGAAGATCCTTTGATCTTTTCTACGGGGTCTGACGCTCAGTGGA | | | |
| | | ACGAAAACTCACGTTAAGGGATTTTGGTCATGAGATTATCAAAAAGG | | | |
| | | ATCTTCACCTAGATCCTTTTAAATTAAAAATGAAGTTTTAAATCAATCT | | | |
| | | AAAGTATATATGAGTAAACTTGGTCTGACAGTTACCAATGCTTAATCA | | | |
| | | GTGAGGCACCTATCTCAGCGATCTGTCTATTTCGTTCATCCATAGTTG | | | |
| | | CCTGACTCCCCGTCGTGTAGATAACTACGATACGGGAGGGCTTACCA | | | |
| | | TCTGGCCCCAGTGCTGCAATGATACCGCGAGACCCACGCTCACCGGC | | | |
| | | TCCAGATTTATCAGCAATAAACCAGCCAGCCGGAAGGGCCGAGCGCA | | | |
| | | GAAGTGGTCCTGCAACTTTATCCGCCTCCATCCAGTCTATTAATTGTTG | | | |
| | | CCGGGAAGCTAGAGTAAGTAGTTCGCCAGTTAATAGTTTGCGCAACG | | | |
| | | TTGTTGCCATTGCTACAGGCATCGTGGTGTCACGCTCGTCGTTTGGTA | | | |
| | | TGGCTTCATTCAGCTCCGGTTCCCAACGATCAAGGCGAGTTACATGAT | | | |
| | | CCCCCATGTTGTGCAAAAAAGCGGTTAGCTCCTTCGGTCCTCCGATCG | | | |
| | | TTGTCAGAAGTAAGTTGGCCGCAGTGTTATCACTCATGGTTATGGCA | | | |
| | | GCACTGCATAATTCTCTTACTGTCATGCCATCCGTAAGATGCTTTTCTG | | | |
| | | TGACTGGTGAGTACTCAACCAAGTCATTCTGAGAATAGTGTATGCGG | | | |
| | | CGACCGAGTTGCTCTTGCCCGGCGTCAATACGGGATAATACCGCGCC | | | |
| | | ACATAGCAGAACTTTAAAAGTGCTCATCATTGGAAAACGTTCTTCGGG | | | |
| | | GCGAAAACTCTCAAGGATCTTACCGCTGTTGAGATCCAGTTCGATGTA | | | |
| | | ACCCACTCGTGCACCCAACTGATCTTCAGCATCTTTTACTTTCACCAGC | | | |
| | | GTTTCTGGGTGAGCAAAAACAGGAAGGCAAAATGCCGCAAAAAAGG | | | |
| | | GAATAAGGGCGACACGGAAATGTTGAATACTCATACTCTTCCTTTTTC | | | |
| | | AATATTATTGAAGCATTTATCAGGGTTATTGTCTCATGAGCGGATACA | | | |
| | | TATTTGAATGTATTTAGAAAAATAAACAAATAGGGGTTCCGCGCACAT | | | |
| | | TTCCCCGAAAAGTGCCACCTGACGTCTAAGAAACCATTATTATCATGA | | | |
| | | CATTAACCTATAAAAATAGGCGTATCACGAGGCCCTTTCGTCTCGCGC | | | |
| | | GTTTCGGTGATGACGGTGAAAACCTCTGACACATGCAGCTCCCGGAG | | | |
| | | ACGGTCACAGCTTGTCTGTAAGCGGATGCCGGGAGCAGACAAGCCC | | | |
| | | GTCAGGGCGCGTCAGCGGGTGTTGGCGGGTGTCGGGGCTGGCTTAA | | | |
| | | CTATGCGGCATCAGAGCAGATTGTACTGAGAGTGCACCATATGCGGT | | | |
| | | GTGAAATACCGCACAGATGCGTAAGGAGAAAATACCGCATCAGGCG | | | |
| | | CCATTCGCCATTCAGGCTGCGCAACTGTTGGGAAGGGCGATCGGTGC | | | |
| | | GGGCCTCTTCGCTATTACGCCAGCTGGCGAAAGGGGGATGTGCTGCA | | | |
| | | AGGCGATTAAGTTGGGTAACGCCAGGGTTTTCCCAGTCACGACGTTG | | | |
| | | TAAAACGACGGCCAGT | | | |
| DRIVER_ | PLV10053 | GAATTCGAGCTTGCATGCCTGCAGGTCGTTACATAACTTACGGTAAAT | 321 | N/A | |
| pCMV_R- | | GGCCCGCCTGGCTGACCGCCCAACGACCCCCGCCCATTGACGTCAAT | | | |
| U5-PBS*- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| LTR_v2 | | AAGTGTATCATATGCCAAGTACGCCCCCTATTGACGTCAATGACGGTA | | | |
| | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGCTTTGATCAGAATGAATTT | | | |
| | | GTCTTGGCTCCGTTTCTTCTTTCGCCCCGTCTAGATTCCTCTCTTACAGC | | | |
| | | TCGAGTGGCCTTCTCAGTCGAACCGTTCACGTTGCGAGCTGCTGGCG | | | |
| | | GCCGCAACATTTTAGCGACTAAACACATCACTGAAGAATCCGAGAGA | | | |
| | | GATGCTAAGAGGAACGCTGCTTTGGAGCTCCACAGGAAAGGATCTTC | | | |
| | | GTATCGGACATCGGAGCAACGGACAGGTACACATGCTAGCGCTAGCT | | | |
| | | TAAAATTTCAGTTTTGTAAAGTGTTGCTGAGGATGTGGTAGGATACG | | | |
| | | AATTAAGCTTGAATCAGTGCTAACCCAACGCTGGTTCTGCTTGGGTCA | | | |
| | | GCAGCGTGTTAATCGGAACTAGAAACGGAAACAGGCAGGTTAGCCG | | | |
| | | CAGCTTTTTAGGAAGCTGCTTAGGTGGAAGAAGAAAGGGTTTAAAGT | | | |
| | | CATGGATCAGGCGGTTGCCCATAGTTTTCAGGAGTTGTTTCAGGCCA | | | |
| | | GAGGAGTAAGGCTTGAAGTACAATTAGTAAAAAATTTTTTAGGTAAG | | | |
| | | ATAGATAGCTGTTGCCCATGGTTCAAGGAAGAAGAAACACTAGATTG | | | |
| | | TGGAACCTGGGAGAAAGTTGGTGAGGCCTTAAAAATCACTCAGGCA | | | |
| | | GATAATTTTACCCTAGGCCTCTGGGCACTCATAAATGATGCAATAAAA | | | |
| | | GATGCCACTTCCCCAGGGCTAAGTTGCCCCCAGGCGGAGCTTGTGGT | | | |
| | | ATCTCAGGAGGAGTGCCTGTCAGAGAGGGCCTCCTCAGAAAAAGATC | | | |
| | | TTCTTAACTCAAAAATTGATAAATGTGGAAACTCGGATGAAAAACTG | | | |
| | | ATTTTTAACAAAAATCACTCAGATAGAGGAGCTGCCCATTACCTTAAT | | | |
| | | GAGAATTGGTCCTCTTGTGAATCTCCTGCTCAACCTGTAGTCCCCACTT | | | |
| | | CGGGAGGTGCCACTCATAGGGACACACGACTAAGCGAGTTAGAGTTT | | | |
| | | GAGATTAAGCTTCAGAGGCTGACTAATGAGCTTCGGGAACTAAAAAA | | | |
| | | GATGTCAGAAGCGGAGAAGAGTAACTCTTCTGTAGTTCACCAGGTGC | | | |
| | | CGCTAGAAAAGGTTGTGAGTCAGGCTCATGGGAAAGGACAGAATAT | | | |
| | | CTCTAATACGCTAGCCTTTCCTGTGGTTGAGGTAGTTGATCAGCAAGA | | | |
| | | TACTAGGGGCAGACATTACCAGACCTTAGATTTCAAGTTGATAAAAG | | | |
| | | AGTTAAAGGCGGCTGTTGTGCAATATGGCCCTTCAGCCCCATTCACTC | | | |
| | | AAGCATTACTGGACACAGTTGTGGAGTCACACTTAACCCCTTTAGATT | | | |
| | | GGAAGACTCTTTCTAAGGCTACCCTGTCAGGAGGAGATTTTTTGCTTT | | | |
| | | GGGATTCTGAATGGCGAGACGCCAGTAAGAAAACTGCTGCTTCTAAC | | | |
| | | GCTCAGGCTGGTAATTCAGACTGGGATAGCAACATGCTTTTAGGAGA | | | |
| | | GGGCCCTTATGAGGGACAGACAAATCAGATTGATTTTCCCGTTGCAG | | | |
| | | TGTACGCGCAAATTGCGACGGCCGCACGCCGTGCTTGGGGAAGGTT | | | |
| | | GCCAGTCAAAGGAGAGATTGGTGGAAGTTTAGCTAGCATTCGGCAG | | | |
| | | AGTTCTGATGAACCATATCAGGATTTTGTGGACAGGCTATTGATTTCA | | | |
| | | GCTAGTAGAATCCTTGGAAATCCGGACACGGGAAGTCCTTTCGTTAT | | | |
| | | GCAATTGGCTTATGAGAATGCTAACGCAATTTGCCGAGCTGCGATTC | | | |
| | | AACCGCATAAGGGAACGACAGATTTGGCGGGATATGTCCGTCTTTGC | | | |
| | | GCAGACATCGGGCCTTCCTGCGAGACCTTGCAGGGAACCCACGCGCA | | | |
| | | GGCAATGTTCTCTAGGAAACGAGGGAATAGTGCATGCTTTAAATGTG | | | |
| | | GAAGTTTAGATCATTTTAGAATTGATTGTCCTCAGAACAAGGGCGCC | | | |
| | | GAGGTTAGACAAACAGGCCGTGCCCCGGGAATATGTCCCCGATGTG | | | |
| | | GAAAGGGCCGCCACTGGGCGAAAGATTGCAAGCATAAAACGAGGGT | | | |
| | | TTTGAGCCGCCCGGTGCCGGGAAACGAGGAAAGGGGTCAGCCCCAG | | | |
| | | GCCCCAAGTTACTCAAAGAAGACAGCTTATGGGGCTCTAAATCTGCT | | | |
| | | GCCCAGCCAACAAGATCAGTTCTTGAGCTTGTCAGGTCAAACCCAGG | | | |
| | | AAACGCAAGACTGGACCTCTGTTCCACTGTCCATGCAGCATTAACCCC | | | |
| | | AGAAGTGGGAGTCCAAACTCTGCCTACCGGAGTCTTTGGACCACTAC | | | |
| | | CTGTAGGAACCTGTGGTTTTCTCTTAGGACGAAGCAGTTCTATTGTAG | | | |
| | | AAGGCCTGCAGATTTATCCAGGTGTTATAAGTAATGATTATGAGGGA | | | |
| | | GAAATTAAAATCATAGCCGCTTGCCCTCGTGGTGCTATAACTATACCC | | | |
| | | GCTAATCAGAAAATTGCTCAACTTACCTTGATCCCCTTGCGCTGGTCA | | | |
| | | CTATCTAAATTCTCTAAAAATGAAGAAGGACAGATTAACTTTGACTCC | | | |
| | | TCTGGCGTAAATTGGGTGAAATCTATCACTAATCAGAGACCTAACCTT | | | |
| | | AAATTGATTCTTGATGGAAAAAGCTTTGAAGGATTAATAGATACCGG | | | |
| | | GGCCGATGTAACCATTATTAGAGGGCAGGACTGGCCCTCAAACTGGC | | | |
| | | CCCTGTCTGTTTCCTTGACTCACCTTCAAGGAATTGGTTATGCCAGTAA | | | |
| | | CCCAAAACGTAGTTCCAAATTGCTAACCTGGAGAGATGAGGATGGAA | | | |
| | | AATCAGGAAATATTCAGCCGTATGTTATGCAAAATTTGCCTGTAACCC | | | |
| | | TGTGGGGAAGAGATCTGTTGTCACAGATGGGCGTTATCCTGTGCAGT | | | |
| | | TCTAAGGAAATGGTGACTGAACAGACGTTCAGGCAGGGACCCCTGCC | | | |
| | | TGATCGTGGACTAATAAAGAAGGGACAGAAAATTAAGACTTTTGAAG | | | |
| | | ATCTTAAACCCCACTCTAACGTGAGAGGTTTAAAGTATTTTCAGTAGT | | | |
| | | GGCCGCTGTCTTGCCTGCATCCCACGCCGAAAAAATTCAATGGCGTA | | | |
| | | ATGATATTCCGGTGTGGGTAGATCAGTGGTCTTTACCTAAAGAGAAA | | | |
| | | ATAGAGGCCGCTTCTCTGCTAGTGCAGGAGCAGTTAGAAGCAGGACA | | | |
| | | TTTGGTGGAGTCTCATTCTCCCTGGAATACACCCATTTTCATTATCAGG | | | |
| | | AAGAAATCGGGAAAATGGAGACTGTTGCAAGATTTAAGAAAGGTTA | | | |
| | | ATGAAACCATGGTACTTATGGGAACTTTACAACCGGGGCTCCCCTCCC | | | |
| | | CAGTAGCCATTCCTAAGGGATACTATAAGATTGTTATAGATTTGAAAG | | | |
| | | ATTGTTTCTTTACCATCCCTTTGCATCCAGAGGATTGTGAGAGATTTGC | | | |
| | | TTTTAGTGTTCCTTCTGTAAATTTCAAGGAACCCATGAAAAGATATCA | | | |
| | | ATGGACAGTTCTCCCGCAGGGGATGGCTAATAGTCCCACCTTATGTCA | | | |
| | | AAAGTTTGTGGCAAAGGCAATTCAGCCTGTTAGACAACAATGGCCAA | | | |
| | | ATATTTACATCATTCATTTCACAGATGATGTTTTGATGGGGGAAAGG | | | |
| | | ACCCCCAAGATTTGCTTTTGTGTTATGGAGACTTACGAAAGGCCCTGG | | | |
| | | CTGATAAGGGATTACAAATTGCTTCTGAAAAGATACAAACTCAGGAT | | | |
| | | CCTTATAATTATTTGGGTTTTAGACTCACTGACCAAGCTGTTTTTCACC | | | |
| | | AGAAAATTGTTATTCGTAGAGATAACTTAAGGACCTTAAATGATTTTC | | | |
| | | AAAAATTGTTAGGTGATATAAACTGGCTTCGCCCCTATCTAAAGCTTA | | | |
| | | CTACAGGGGAGTTGAAACCTTTATTTGATATTCTTAAAGGGAGTTCTG | | | |
| | | ATCCTACTTCCCCTAGATCCCTAACCTCAGAAGGTTTACTGGCCTTACA | | | |
| | | GCTAGTGGAAAAGGCTATTGAAGAACAGTTTGTCACTTACATAGATT | | | |
| | | ACTCCCTGCCGCTGCACCTGTTAATTTTTAACACGACTCATGTGCCTAC | | | |
| | | GGGATTGCTATGGCAAAAATTTCCTATAATGTGGATACATTCAAGGAT | | | |
| | | TTCTCCCAAACGTAATATTTTGCCATATCATGAAGCAGTGGCTCAGAT | | | |
| | | GATTATCACTGGAAGAAGGCAGGCATTGACTTATTTTGGAAAGGAGC | | | |
| | | CAGATATCATTGTCCAGCCTTACAGCGTGAGTCAGGACACTTGGCTG | | | |
| | | AAACAGCATAGTACAGATTGGTTGCTTGCACAATTAGGGTTTGAAGG | | | |
| | | AACTATAGATAGCCACTACCCCCAAGATAGGTTGATAAAATTCTTAAA | | | |
| | | TGTACATGATATGATATTTCCTAAGATGACTTCCTTACAGCCTTTAAAT | | | |
| | | AATGCTCTATTGATTTTTACTGATGGCTCCTCTAAAGGGCGAGCTGGA | | | |
| | | TATCTTATTAGTAATCAACAGGTTATCGTAGAGACTCCTGGTCTCTCG | | | |
| | | GCTCAGCTCGCCGAACTAACAGCAGTACTGAAGGTTTTTCAGTCTGTA | | | |
| | | CAGGAGGCTTTTAATATTTTTACTGACAGTTTATATGTTGCTCAGTCA | | | |
| | | GTACCCTTATTGGAAACCTGTGGTACTTTTAACTTCAATACGCCGTCA | | | |
| | | GGATCTTTATTTTCAGAATTACAAAACATCATTCTCGCCCGGAAAAAT | | | |
| | | CCGTTTTATATTGGCCACATACGGTCTCACTCTGGTCTTCCTGGACCTC | | | |
| | | TGGCAGAGGGTAATAATTGCATTGACAGAGCTCTAATAGGAGAAGCC | | | |
| | | TTAGTTTCAGATCGGGTTGCTTTGGCCCAACGTGATCATGAAAGGTTT | | | |
| | | CATCTCTCTAGCCATACCCTAAGGCTCCGACATAAGATCACCAAGGAG | | | |
| | | CAAGCGAGAATGATTGTAAAACAATGTCCTAAATGTATTACTTTATCT | | | |
| | | CCAGTGCCGCATCTAGGAGTTAATCCTAGAGGCCTTATGCCTAATCAT | | | |
| | | ATTTGGCAAATGGATATAACCCATTATGCAGAATTTGGAAAACTAAA | | | |
| | | ATATATACATGTTTGCATTGATACTTGTTCAGGATTTCTCTTTGCTTCTC | | | |
| | | TGCATACAGGAGAAGCTTCAAAAAACGTAATTGATCATTGCCTACAA | | | |
| | | GCATTTAATGCCATGGGATTACCTAAACTTATTAAGACAGACAATGG | | | |
| | | GCCATCTTATTCCAGTAAAAACTTTATTTCATTCTGTAAAGAATTCGGT | | | |
| | | ATTAAACATAAAACTGGAATTCCTTACAACCCCATGGGACAAGGAAT | | | |
| | | AGTTGAACGTGCTCATCGCACCTTAAAGAATTGGCTCTTTAAGACAAA | | | |
| | | AGAGGGGCAGCTATATCCCCCAAGGTCTCCAAAGGCCCACCTTGCCT | | | |
| | | TCACCTTATTTGTCCTAAATTTCTTGCACACCGATATCAAGGGCCAGTC | | | |
| | | TGCAGCGGATCGCCACTGGCATCCAGTTACTTCTAATTCTTATGCATT | | | |
| | | GGTAAAATGGAAGGACCCCCTGACTAATGAATGGAAGGGTCCAGAT | | | |
| | | CCAGTTCTAATTTGGGGTAGAGGCTCAGTTTGTGTTTTTTCACGAGAT | | | |
| | | GAAGATGGAGCACGGTGGCTGCCAGAGAGATTAATTCGTCAGACGA | | | |
| | | ACACAGATTCTGACTCTTCTGGTAAGTATCATTCTAAAGACTAAAATT | | | |
| | | CCTTTTGTGCTTAAAATTCAGCTGAGAGCAACAGCTCTCAAAGCTGTT | | | |
| | | CTCCAGCTACTCTCTGAGCCAGCTCCCGACAGGAGGCCGGAGACTAG | | | |
| | | CCTCAGCTTTACAATTTGCATTTAAATAAAGTACCTAGACTTCCCCGAA | | | |
| | | AAAAGTTCTGCTTTTCTACTTTCTCACTGTCTTTCAAGATTTTGTCTTTC | | | |
| | | AAGCAGGTAAATCAACATTCTCGAGGCGGACCAGCGGATGTGCATCC | | | |
| | | CCGCCCCCCTAGAGCACTCAGGTGGCAGCTGTTATCCCCAGTCTCAGG | | | |
| | | ACATTCCAGCATGTGGCCTTCAGTCTGAGTTAAAAATTAGGTTTACCC | | | |
| | | AGAGGACTAGAATAGTAGATATTTCTATATTAATAAAGATTGGTTTTT | | | |
| | | ATTTTGATAGACAGGCTTAGCCCCTTAGCTGACCTCTGGCTTTTCACCC | | | |
| | | TTGCTGTTACTGCAAGGTGTCTTTAGCTCAATAAGGCTGTGGAAAAAA | | | |
| | | ACAGGGATGAGGAGGAACGGCTCCCAGCTCCTATTTTAGCCACAAAT | | | |
| | | CGTGGTGTTACTAACGACATAATTCTTGCTTAGGCTTTGCTAAATCTG | | | |
| | | AGGTTGATAATTCTCCTTTAGGAGCTGCACAGCGCTCAGAACTGTGCA | | | |
| | | TACTGATTTGTGATGGTACAAATTCAGTATGGGCATCGCTTGGTGCA | | | |
| | | GATGGAGGTACTGCAAGGAAAGGTCCCAGCTTGACCATTTCTGAGTT | | | |
| | | TCCTGTGAGATAAACCCGGTTTGAAAGAGGTTGGTACCAAATTATAT | | | |
| | | ATCCCTCGGCTCTACCTCGCCTCCCCAAAAGGTACCAGAGCCACAGGT | | | |
| | | GTGGATTTTAACAGAATCCACGGGAGGAATCGGGTCCATGTCCACCC | | | |
| | | AAGCCAAGGTTAAAAGCCCACTCATCTACGGATGAGAAAATCATTTG | | | |
| | | ATCACCTCAGTTAAGCGCTGCCTTATTTTAACTTAATTAATAGGGGGG | | | |
| | | AGAGAGATTGGAGACTTACTATTGAAAGGGCAAGCCCTTCACTGCCT | | | |
| | | CCCACCCAAATAAAAAAGCCAATTGGCCTTGTACTACAGAGCTGGCC | | | |
| | | GGACCCCTTATCCCTGTTACCCACCAATCATCCAAAAATGCGGAGGAA | | | |
| | | TATCAACTTAGTGTTATTCTTATTATAGTGTATTTCACACTTGTTCAGTC | | | |
| | | AAACTTAGCCAGAGTTCCAACGCCCTACTTAAAATTCAACTAGAAAGT | | | |
| | | TACCTACCAAGTACTAATTAGCATTATAAAGTCAGAGCCTACAGCTCC | | | |
| | | AGGCTTTTCAGTTAGTTGTTTACTAAGATAAGAAAAGACAGTCTTAGC | | | |
| | | CAGATACAGTTTACCATAATAAAAGTTAAAGAATCCCAGGGAAGCAA | | | |
| | | GTTTTTTCTTTTAGCCCTAGATTCCAGGCAGAACTATTGAGCATAGAT | | | |
| | | AATTTTTCCCCCTCAGGCCAGCTTTTTCTTTTTTTTTAATTTTGTTAATA | | | |
| | | AAAGGGAGGAGATGTAGTCTCCCCTCCCCCAGCCTGAAACCTGCTTG | | | |
| | | CTCAGGGGTGGAGCTTCCCGCTCATCGCTCTGCCACGCCCACTGCTG | | | |
| | | GAACCTGCGGAGCCACACACGTGCACCTTTCTACTGGACCAGAGATT | | | |
| | | ATTCGGCGGGAATCGGGTCCCCTCCCCCTTCCTTCATAACTAGTGTCC | | | |
| | | CAACAATAAAATTTGAGCTTTGATCAGAATGAATTTGTCTTGGCTCCG | | | |
| | | TTTCTTCTTTCGCCCCGTCTAGATTCCTCTCTTACAGCTCGAGTGGCCT | | | |
| | | TCTCAGTCGAACCGTTCACGTTGCGAGCTGCTGGCGGCCGCAACAGG | | | |
| | | GGATCCAGACATGATAAGATACATTGATGAGTTTGGACAAACCACAA | | | |
| | | CTAGAATGCAGTGAAAAAAATGCTTTATTTGTGAAATTTGTGATGCTA | | | |
| | | TTGCTTTATTTGTAACCATTATAAGCTGCAATAAACAAGTTAACAACA | | | |
| | | ACAATTGCATTCATTTTATGTTTCAGGTTCAGGGGGAGGTGTGGGAG | | | |
| | | GTTTTTTCGGATCCTCTAGAGTCGACCTGCAGGCATGCAAGCTTGGCG | | | |
| | | TAATCATGGTCATAGCTGTTTCCTGTGTGAAATTGTTATCCGCTCACAA | | | |
| | | TTCCACACAACATACGAGCCGGAAGCATAAAGTGTAAAGCCTGGGGT | | | |
| | | GCCTAATGAGTGAGCTAACTCACATTAATTGCGTTGCGCTCACTGCCC | | | |
| | | GCTTTCCAGTCGGGAAACCTGTCGTGCCAGCTGCATTAATGAATCGG | | | |
| | | CCAACGCGCGGGGAGAGGCGGTTTGCGTATTGGGCGCTCTTCCGCTT | | | |
| | | CCTCGCTCACTGACTCGCTGCGCTCGGTCGTTCGGCTGCGGCGAGCG | | | |
| | | GTATCAGCTCACTCAAAGGCGGTAATACGGTTATCCACAGAATCAGG | | | |
| | | GGATAACGCAGGAAAGAACATGTGAGCAAAAGGCCAGCAAAAGGCC | | | |
| | | AGGAACCGTAAAAAGGCCGCGTTGCTGGCGTTTTTCCATAGGCTCCG | | | |
| | | CCCCCCTGACGAGCATCACAAAAATCGACGCTCAAGTCAGAGGTGGC | | | |
| | | GAAACCCGACAGGACTATAAAGATACCAGGCGTTTCCCCCTGGAAGC | | | |
| | | TCCCTCGTGCGCTCTCCTGTTCCGACCCTGCCGCTTACCGGATACCTGT | | | |
| | | CCGCCTTTCTCCCTTCGGGAAGCGTGGCGCTTTCTCATAGCTCACGCT | | | |
| | | GTAGGTATCTCAGTTCGGTGTAGGTCGTTCGCTCCAAGCTGGGCTGT | | | |
| | | GTGCACGAACCCCCCGTTCAGCCCGACCGCTGCGCCTTATCCGGTAAC | | | |
| | | TATCGTCTTGAGTCCAACCCGGTAAGACACGACTTATCGCCACTGGCA | | | |
| | | GCAGCCACTGGTAACAGGATTAGCAGAGCGAGGTATGTAGGCGGTG | | | |
| | | CTACAGAGTTCTTGAAGTGGTGGCCTAACTACGGCTACACTAGAAGG | | | |
| | | ACAGTATTTGGTATCTGCGCTCTGCTGAAGCCAGTTACCTTCGGAAAA | | | |
| | | AGAGTTGGTAGCTCTTGATCCGGCAAACAAACCACCGCTGGTAGCGG | | | |
| | | TGGTTTTTTTGTTTGCAAGCAGCAGATTACGCGCAGAAAAAAAGGAT | | | |
| | | CTCAAGAAGATCCTTTGATCTTTTCTACGGGGTCTGACGCTCAGTGGA | | | |
| | | ACGAAAACTCACGTTAAGGGATTTTGGTCATGAGATTATCAAAAAGG | | | |
| | | ATCTTCACCTAGATCCTTTTAAATTAAAAATGAAGTTTTAAATCAATCT | | | |
| | | AAAGTATATATGAGTAAACTTGGTCTGACAGTTACCAATGCTTAATCA | | | |
| | | GTGAGGCACCTATCTCAGCGATCTGTCTATTTCGTTCATCCATAGTTG | | | |
| | | CCTGACTCCCCGTCGTGTAGATAACTACGATACGGGAGGGCTTACCA | | | |
| | | TCTGGCCCCAGTGCTGCAATGATACCGCGAGACCCACGCTCACCGGC | | | |
| | | TCCAGATTTATCAGCAATAAACCAGCCAGCCGGAAGGGCCGAGCGCA | | | |
| | | GAAGTGGTCCTGCAACTTTATCCGCCTCCATCCAGTCTATTAATTGTTG | | | |
| | | CCGGGAAGCTAGAGTAAGTAGTTCGCCAGTTAATAGTTTGCGCAACG | | | |
| | | TTGTTGCCATTGCTACAGGCATCGTGGTGTCACGCTCGTCGTTTGGTA | | | |
| | | TGGCTTCATTCAGCTCCGGTTCCCAACGATCAAGGCGAGTTACATGAT | | | |
| | | CCCCCATGTTGTGCAAAAAAGCGGTTAGCTCCTTCGGTCCTCCGATCG | | | |
| | | TTGTCAGAAGTAAGTTGGCCGCAGTGTTATCACTCATGGTTATGGCA | | | |
| | | GCACTGCATAATTCTCTTACTGTCATGCCATCCGTAAGATGCTTTTCTG | | | |
| | | TGACTGGTGAGTACTCAACCAAGTCATTCTGAGAATAGTGTATGCGG | | | |
| | | CGACCGAGTTGCTCTTGCCCGGCGTCAATACGGGATAATACCGCGCC | | | |
| | | ACATAGCAGAACTTTAAAAGTGCTCATCATTGGAAAACGTTCTTCGGG | | | |
| | | GCGAAAACTCTCAAGGATCTTACCGCTGTTGAGATCCAGTTCGATGTA | | | |
| | | ACCCACTCGTGCACCCAACTGATCTTCAGCATCTTTTACTTTCACCAGC | | | |
| | | GTTTCTGGGTGAGCAAAAACAGGAAGGCAAAATGCCGCAAAAAAGG | | | |
| | | GAATAAGGGCGACACGGAAATGTTGAATACTCATACTCTTCCTTTTTC | | | |
| | | AATATTATTGAAGCATTTATCAGGGTTATTGTCTCATGAGCGGATACA | | | |
| | | TATTTGAATGTATTTAGAAAAATAAACAAATAGGGGTTCCGCGCACAT | | | |
| | | TTCCCCGAAAAGTGCCACCTGACGTCTAAGAAACCATTATTATCATGA | | | |
| | | CATTAACCTATAAAAATAGGCGTATCACGAGGCCCTTTCGTCTCGCGC | | | |
| | | GTTTCGGTGATGACGGTGAAAACCTCTGACACATGCAGCTCCCGGAG | | | |
| | | ACGGTCACAGCTTGTCTGTAAGCGGATGCCGGGAGCAGACAAGCCC | | | |
| | | GTCAGGGCGCGTCAGCGGGTGTTGGCGGGTGTCGGGGCTGGCTTAA | | | |
| | | CTATGCGGCATCAGAGCAGATTGTACTGAGAGTGCACCATATGCGGT | | | |
| | | GTGAAATACCGCACAGATGCGTAAGGAGAAAATACCGCATCAGGCG | | | |
| | | CCATTCGCCATTCAGGCTGCGCAACTGTTGGGAAGGGCGATCGGTGC | | | |
| | | GGGCCTCTTCGCTATTACGCCAGCTGGCGAAAGGGGGATGTGCTGCA | | | |
| | | AGGCGATTAAGTTGGGTAACGCCAGGGTTTTCCCAGTCACGACGTTG | | | |
| | | TAAAACGACGGCCAGT | | | |
| DRIVER_ | PLV10054 | GAATTCGAGCTTGCATGCCTGCAGGTCGTTACATAACTTACGGTAAAT | 322 | N/A | |
| pCMV_R- | | GGCCCGCCTGGCTGACCGCCCAACGACCCCCGCCCATTGACGTCAAT | | | |
| U5-PBS*- | | AATGACGTATGTTCCCATAGTAACGCCAATAGGGACTTTCCATTGACG | | | |
| MusD6- | | TCAATGGGTGGAGTATTTACGGTAAACTGCCCACTTGGCAGTACATC | | | |
| LTR_v3 | | AAGTGTATCATATGCCAAGTACGCCCCCTATTGACGTCAATGACGGTA | | | |
| | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTGAGCTTTGATCAGAATGAATTTGTCTTGGCTCCGTTT | | | |
| | | CTTCTTTCGCCCCGTCTAGATTCCTCTCTTACAGCTCGAGTGGCCTTCT | | | |
| | | CAGTCGAACCGTTCACGTTGCGAGCTGCTGGCGGCCGCAACATTTTA | | | |
| | | GCGACTAAACACATCACTGAAGAATCCGAGAGAGATGCTAAGAGGA | | | |
| | | ACGCTGCTTTGGAGCTCCACAGGAAAGGATCTTCGTATCGGACATCG | | | |
| | | GAGCAACGGACAGGTACACATGCTAGCGCTAGCTTAAAATTTCAGTT | | | |
| | | TTGTAAAGTGTTGCTGAGGATGTGGTAGGATACGAATTAAGCTTGAA | | | |
| | | TCAGTGCTAACCCAACGCTGGTTCTGCTTGGGTCAGCAGCGTGTTAAT | | | |
| | | CGGAACTAGAAACGGAAACAGGCAGGTTAGCCGCAGCTTTTTAGGA | | | |
| | | AGCTGCTTAGGTGGAAGAAGAAAGGGTTTAAAGTCATGGATCAGGC | | | |
| | | GGTTGCCCATAGTTTTCAGGAGTTGTTTCAGGCCAGAGGAGTAAGGC | | | |
| | | TTGAAGTACAATTAGTAAAAAATTTTTTAGGTAAGATAGATAGCTGTT | | | |
| | | GCCCATGGTTCAAGGAAGAAGAAACACTAGATTGTGGAACCTGGGA | | | |
| | | GAAAGTTGGTGAGGCCTTAAAAATCACTCAGGCAGATAATTTTACCCT | | | |
| | | AGGCCTCTGGGCACTCATAAATGATGCAATAAAAGATGCCACTTCCCC | | | |
| | | AGGGCTAAGTTGCCCCCAGGCGGAGCTTGTGGTATCTCAGGAGGAG | | | |
| | | TGCCTGTCAGAGAGGGCCTCCTCAGAAAAAGATCTTCTTAACTCAAAA | | | |
| | | ATTGATAAATGTGGAAACTCGGATGAAAAACTGATTTTTAACAAAAA | | | |
| | | TCACTCAGATAGAGGAGCTGCCCATTACCTTAATGAGAATTGGTCCTC | | | |
| | | TTGTGAATCTCCTGCTCAACCTGTAGTCCCCACTTCGGGAGGTGCCAC | | | |
| | | TCATAGGGACACACGACTAAGCGAGTTAGAGTTTGAGATTAAGCTTC | | | |
| | | AGAGGCTGACTAATGAGCTTCGGGAACTAAAAAAGATGTCAGAAGC | | | |
| | | GGAGAAGAGTAACTCTTCTGTAGTTCACCAGGTGCCGCTAGAAAAGG | | | |
| | | TTGTGAGTCAGGCTCATGGGAAAGGACAGAATATCTCTAATACGCTA | | | |
| | | GCCTTTCCTGTGGTTGAGGTAGTTGATCAGCAAGATACTAGGGGCAG | | | |
| | | ACATTACCAGACCTTAGATTTCAAGTTGATAAAAGAGTTAAAGGCGG | | | |
| | | CTGTTGTGCAATATGGCCCTTCAGCCCCATTCACTCAAGCATTACTGG | | | |
| | | ACACAGTTGTGGAGTCACACTTAACCCCTTTAGATTGGAAGACTCTTT | | | |
| | | CTAAGGCTACCCTGTCAGGAGGAGATTTTTTGCTTTGGGATTCTGAAT | | | |
| | | GGCGAGACGCCAGTAAGAAAACTGCTGCTTCTAACGCTCAGGCTGGT | | | |
| | | AATTCAGACTGGGATAGCAACATGCTTTTAGGAGAGGGCCCTTATGA | | | |
| | | GGGACAGACAAATCAGATTGATTTTCCCGTTGCAGTGTACGCGCAAA | | | |
| | | TTGCGACGGCCGCACGCCGTGCTTGGGGAAGGTTGCCAGTCAAAGG | | | |
| | | AGAGATTGGTGGAAGTTTAGCTAGCATTCGGCAGAGTTCTGATGAAC | | | |
| | | CATATCAGGATTTTGTGGACAGGCTATTGATTTCAGCTAGTAGAATCC | | | |
| | | TTGGAAATCCGGACACGGGAAGTCCTTTCGTTATGCAATTGGCTTATG | | | |
| | | AGAATGCTAACGCAATTTGCCGAGCTGCGATTCAACCGCATAAGGGA | | | |
| | | ACGACAGATTTGGCGGGATATGTCCGTCTTTGCGCAGACATCGGGCC | | | |
| | | TTCCTGCGAGACCTTGCAGGGAACCCACGCGCAGGCAATGTTCTCTA | | | |
| | | GGAAACGAGGGAATAGTGCATGCTTTAAATGTGGAAGTTTAGATCAT | | | |
| | | TTTAGAATTGATTGTCCTCAGAACAAGGGCGCCGAGGTTAGACAAAC | | | |
| | | AGGCCGTGCCCCGGGAATATGTCCCCGATGTGGAAAGGGCCGCCACT | | | |
| | | GGGCGAAAGATTGCAAGCATAAAACGAGGGTTTTGAGCCGCCCGGT | | | |
| | | GCCGGGAAACGAGGAAAGGGGTCAGCCCCAGGCCCCAAGTTACTCA | | | |
| | | AAGAAGACAGCTTATGGGGCTCTAAATCTGCTGCCCAGCCAACAAGA | | | |
| | | TCAGTTCTTGAGCTTGTCAGGTCAAACCCAGGAAACGCAAGACTGGA | | | |
| | | CCTCTGTTCCACTGTCCATGCAGCATTAACCCCAGAAGTGGGAGTCCA | | | |
| | | AACTCTGCCTACCGGAGTCTTTGGACCACTACCTGTAGGAACCTGTGG | | | |
| | | TTTTCTCTTAGGACGAAGCAGTTCTATTGTAGAAGGCCTGCAGATTTA | | | |
| | | TCCAGGTGTTATAAGTAATGATTATGAGGGAGAAATTAAAATCATAG | | | |
| | | CCGCTTGCCCTCGTGGTGCTATAACTATACCCGCTAATCAGAAAATTG | | | |
| | | CTCAACTTACCTTGATCCCCTTGCGCTGGTCACTATCTAAATTCTCTAA | | | |
| | | AAATGAAGAAGGACAGATTAACTTTGACTCCTCTGGCGTAAATTGGG | | | |
| | | TGAAATCTATCACTAATCAGAGACCTAACCTTAAATTGATTCTTGATG | | | |
| | | GAAAAAGCTTTGAAGGATTAATAGATACCGGGGCCGATGTAACCATT | | | |
| | | ATTAGAGGGCAGGACTGGCCCTCAAACTGGCCCCTGTCTGTTTCCTTG | | | |
| | | ACTCACCTTCAAGGAATTGGTTATGCCAGTAACCCAAAACGTAGTTCC | | | |
| | | AAATTGCTAACCTGGAGAGATGAGGATGGAAAATCAGGAAATATTCA | | | |
| | | GCCGTATGTTATGCAAAATTTGCCTGTAACCCTGTGGGGAAGAGATC | | | |
| | | TGTTGTCACAGATGGGCGTTATCCTGTGCAGTTCTAAGGAAATGGTG | | | |
| | | ACTGAACAGACGTTCAGGCAGGGACCCCTGCCTGATCGTGGACTAAT | | | |
| | | AAAGAAGGGACAGAAAATTAAGACTTTTGAAGATCTTAAACCCCACT | | | |
| | | CTAACGTGAGAGGTTTAAAGTATTTTCAGTAGTGGCCGCTGTCTTGCC | | | |
| | | TGCATCCCACGCCGAAAAAATTCAATGGCGTAATGATATTCCGGTGT | | | |
| | | GGGTAGATCAGTGGTCTTTACCTAAAGAGAAAATAGAGGCCGCTTCT | | | |
| | | CTGCTAGTGCAGGAGCAGTTAGAAGCAGGACATTTGGTGGAGTCTCA | | | |
| | | TTCTCCCTGGAATACACCCATTTTCATTATCAGGAAGAAATCGGGAAA | | | |
| | | ATGGAGACTGTTGCAAGATTTAAGAAAGGTTAATGAAACCATGGTAC | | | |
| | | TTATGGGAACTTTACAACCGGGGCTCCCCTCCCCAGTAGCCATTCCTA | | | |
| | | AGGGATACTATAAGATTGTTATAGATTTGAAAGATTGTTTCTTTACCA | | | |
| | | TCCCTTTGCATCCAGAGGATTGTGAGAGATTTGCTTTTAGTGTTCCTTC | | | |
| | | TGTAAATTTCAAGGAACCCATGAAAAGATATCAATGGACAGTTCTCCC | | | |
| | | GCAGGGGATGGCTAATAGTCCCACCTTATGTCAAAAGTTTGTGGCAA | | | |
| | | AGGCAATTCAGCCTGTTAGACAACAATGGCCAAATATTTACATCATTC | | | |
| | | ATTTCACAGATGATGTTTTGATGGCGGGAAAGGACCCCCAAGATTTG | | | |
| | | CTTTTGTGTTATGGAGACTTACGAAAGGCCCTGGCTGATAAGGGATT | | | |
| | | ACAAATTGCTTCTGAAAAGATACAAACTCAGGATCCTTATAATTATTT | | | |
| | | GGGTTTTAGACTCACTGACCAAGCTGTTTTTCACCAGAAAATTGTTAT | | | |
| | | TCGTAGAGATAACTTAAGGACCTTAAATGATTTTCAAAAATTGTTAGG | | | |
| | | TGATATAAACTGGCTTCGCCCCTATCTAAAGCTTACTACAGGGGAGTT | | | |
| | | GAAACCTTTATTTGATATTCTTAAAGGGAGTTCTGATCCTACTTCCCCT | | | |
| | | AGATCCCTAACCTCAGAAGGTTTACTGGCCTTACAGCTAGTGGAAAA | | | |
| | | GGCTATTGAAGAACAGTTTGTCACTTACATAGATTACTCCCTGCCGCT | | | |
| | | GCACCTGTTAATTTTTAACACGACTCATGTGCCTACGGGATTGCTATG | | | |
| | | GCAAAAATTTCCTATAATGTGGATACATTCAAGGATTTCTCCCAAACG | | | |
| | | TAATATTTTGCCATATCATGAAGCAGTGGCTCAGATGATTATCACTGG | | | |
| | | AAGAAGGCAGGCATTGACTTATTTTGGAAAGGAGCCAGATATCATTG | | | |
| | | TCCAGCCTTACAGCGTGAGTCAGGACACTTGGCTGAAACAGCATAGT | | | |
| | | ACAGATTGGTTGCTTGCACAATTAGGGTTTGAAGGAACTATAGATAG | | | |
| | | CCACTACCCCCAAGATAGGTTGATAAAATTCTTAAATGTACATGATAT | | | |
| | | GATATTTCCTAAGATGACTTCCTTACAGCCTTTAAATAATGCTCTATTG | | | |
| | | ATTTTTACTGATGGCTCCTCTAAAGGGCGAGCTGGATATCTTATTAGT | | | |
| | | AATCAACAGGTTATCGTAGAGACTCCTGGTCTCTCGGCTCAGCTCGCC | | | |
| | | GAACTAACAGCAGTACTGAAGGTTTTTCAGTCTGTACAGGAGGCTTTT | | | |
| | | AATATTTTTACTGACAGTTTATATGTTGCTCAGTCAGTACCCTTATTGG | | | |
| | | AAACCTGTGGTACTTTTAACTTCAATACGCCGTCAGGATCTTTATTTTC | | | |
| | | AGAATTACAAAACATCATTCTCGCCCGGAAAAATCCGTTTTATATTGG | | | |
| | | CCACATACGGTCTCACTCTGGTCTTCCTGGACCTCTGGCAGAGGGTAA | | | |
| | | TAATTGCATTGACAGAGCTCTAATAGGAGAAGCCTTAGTTTCAGATCG | | | |
| | | GGTTGCTTTGGCCCAACGTGATCATGAAAGGTTTCATCTCTCTAGCCA | | | |
| | | TACCCTAAGGCTCCGACATAAGATCACCAAGGAGCAAGCGAGAATGA | | | |
| | | TTGTAAAACAATGTCCTAAATGTATTACTTTATCTCCAGTGCCGCATCT | | | |
| | | AGGAGTTAATCCTAGAGGCCTTATGCCTAATCATATTTGGCAAATGGA | | | |
| | | TATAACCCATTATGCAGAATTTGGAAAACTAAAATATATACATGTTTG | | | |
| | | CATTGATACTTGTTCAGGATTTCTCTTTGCTTCTCTGCATACAGGAGAA | | | |
| | | GCTTCAAAAAACGTAATTGATCATTGCCTACAAGCATTTAATGCCATG | | | |
| | | GGATTACCTAAACTTATTAAGACAGACAATGGGCCATCTTATTCCAGT | | | |
| | | AAAAACTTTATTTCATTCTGTAAAGAATTCGGTATTAAACATAAAACT | | | |
| | | GGAATTCCTTACAACCCCATGGGACAAGGAATAGTTGAACGTGCTCA | | | |
| | | TCGCACCTTAAAGAATTGGCTCTTTAAGACAAAAGAGGGGCAGCTAT | | | |
| | | ATCCCCCAAGGTCTCCAAAGGCCCACCTTGCCTTCACCTTATTTGTCCT | | | |
| | | AAATTTCTTGCACACCGATATCAAGGGCCAGTCTGCAGCGGATCGCC | | | |
| | | ACTGGCATCCAGTTACTTCTAATTCTTATGCATTGGTAAAATGGAAGG | | | |
| | | ACCCCCTGACTAATGAATGGAAGGGTCCAGATCCAGTTCTAATTTGG | | | |
| | | GGTAGAGGCTCAGTTTGTGTTTTTTCACGAGATGAAGATGGAGCACG | | | |
| | | GTGGCTGCCAGAGAGATTAATTCGTCAGACGAACACAGATTCTGACT | | | |
| | | CTTCTGGTAAGTATCATTCTAAAGACTAAAATTCCTTTTGTGCTTAAAA | | | |
| | | TTCAGCTGAGAGCAACAGCTCTCAAAGCTGTTCTCCAGCTACTCTCTG | | | |
| | | AGCCAGCTCCCGACAGGAGGCCGGAGACTAGCCTCAGCTTTACAATT | | | |
| | | TGCATTTAAATAAAGTACCTAGACTTCCCCGAAAAAAGTTCTGCTTTTC | | | |
| | | TACTTTCTCACTGTCTTTCAAGATTTTGTCTTTCAAGCAGGTAAATCAA | | | |
| | | CATTCTCGAGGCGGACCAGCGGATGTGCATCCCCGCCCCCCTAGAGC | | | |
| | | ACTCAGGTGGCAGCTGTTATCCCCAGTCTCAGGACATTCCAGCATGTG | | | |
| | | GCCTTCAGTCTGAGTTAAAAATTAGGTTTACCCAGAGGACTAGAATA | | | |
| | | GTAGATATTTCTATATTAATAAAGATTGGTTTTTATTTTGATAGACAGG | | | |
| | | CTTAGCCCCTTAGCTGACCTCTGGCTTTTCACCCTTGCTGTTACTGCAA | | | |
| | | GGTGTCTTTAGCTCAATAAGGCTGTGGAAAAAAACAGGGATGAGGA | | | |
| | | GGAACGGCTCCCAGCTCCTATTTTAGCCACAAATCGTGGTGTTACTAA | | | |
| | | CGACATAATTCTTGCTTAGGCTTTGCTAAATCTGAGGTTGATAATTCTC | | | |
| | | CTTTAGGAGCTGCACAGCGCTCAGAACTGTGCATACTGATTTGTGATG | | | |
| | | GTACAAATTCAGTATGGGCATCGCTTGGTGCAGATGGAGGTACTGCA | | | |
| | | AGGAAAGGTCCCAGCTTGACCATTTCTGAGTTTCCTGTGAGATAAACC | | | |
| | | CGGTTTGAAAGAGGTTGGTACCAAATTATATATCCCTCGGCTCTACCT | | | |
| | | CGCCTCCCCAAAAGGTACCAGAGCCACAGGTGTGGATTTTAACAGAA | | | |
| | | TCCACGGGAGGAATCGGGTCCATGTCCACCCAAGCCAAGGTTAAAAG | | | |
| | | CCCACTCATCTACGGATGAGAAAATCATTTGATCACCTCAGTTAAGCG | | | |
| | | CTGCCTTATTTTAACTTAATTAATAGGGGGGAGAGAGATTGGAGACT | | | |
| | | TACTATTGAAAGGGCAAGCCCTTCACTGCCTCCCACCCAAATAAAAAA | | | |
| | | GCCAATTGGCCTTGTACTACAGAGCTGGCCGGACCCCTTATCCCTGTT | | | |
| | | ACCCACCAATCATCCAAAAATGCGGAGGAATATCAACTTAGTGTTATT | | | |
| | | CTTATTATAGTGTATTTCACACTTGTTCAGTCAAACTTAGCCAGAGTTC | | | |
| | | CAACGCCCTACTTAAAATTCAACTAGAAAGTTACCTACCAAGTACTAA | | | |
| | | TTAGCATTATAAAGTCAGAGCCTACAGCTCCAGGCTTTTCAGTTAGTT | | | |
| | | GTTTACTAAGATAAGAAAAGACAGTCTTAGCCAGATACAGTTTACCAT | | | |
| | | AATAAAAGTTAAAGAATCCCAGGGAAGCAAGTTTTTTCTTTTAGCCCT | | | |
| | | AGATTCCAGGCAGAACTATTGAGCATAGATAATTTTTCCCCCTCAGGC | | | |
| | | CAGCTTTTTCTTTTTTTTTAATTTTGTTAATAAAAGGGAGGAGATGTAG | | | |
| | | TCTCCCCTCCCCCAGCCTGAAACCTGCTTGCTCAGGGGTGGAGCTTCC | | | |
| | | CGCTCATCGCTCTGCCACGCCCACTGCTGGAACCTGCGGAGCCACAC | | | |
| | | ACGTGCACCTTTCTACTGGACCAGAGATTATTCGGCGGGAATCGGGT | | | |
| | | CCCCTCCCCCTTCCTTCATAACTAGTGTCCCAACAATAAAATTTGAGCT | | | |
| | | TTGATCAGAATGAATTTGTCTTGGCTCCGTTTCTTCTTTCGCCCCGTCT | | | |
| | | AGATTCCTCTCTTACAGCTCGAGTGGCCTTCTCAGTCGAACCGTTCAC | | | |
| | | GTTGCGAGCTGCTGGCGGCCGCAACAGGGGATCCAGACATGATAAG | | | |
| | | ATACATTGATGAGTTTGGACAAACCACAACTAGAATGCAGTGAAAAA | | | |
| | | AATGCTTTATTTGTGAAATTTGTGATGCTATTGCTTTATTTGTAACCAT | | | |
| | | TATAAGCTGCAATAAACAAGTTAACAACAACAATTGCATTCATTTTAT | | | |
| | | GTTTCAGGTTCAGGGGGAGGTGTGGGAGGTTTTTTCGGATCCTCTAG | | | |
| | | AGTCGACCTGCAGGCATGCAAGCTTGGCGTAATCATGGTCATAGCTG | | | |
| | | TTTCCTGTGTGAAATTGTTATCCGCTCACAATTCCACACAACATACGA | | | |
| | | GCCGGAAGCATAAAGTGTAAAGCCTGGGGTGCCTAATGAGTGAGCT | | | |
| | | AACTCACATTAATTGCGTTGCGCTCACTGCCCGCTTTCCAGTCGGGAA | | | |
| | | ACCTGTCGTGCCAGCTGCATTAATGAATCGGCCAACGCGCGGGGAGA | | | |
| | | GGCGGTTTGCGTATTGGGCGCTCTTCCGCTTCCTCGCTCACTGACTCG | | | |
| | | CTGCGCTCGGTCGTTCGGCTGCGGCGAGCGGTATCAGCTCACTCAAA | | | |
| | | GGCGGTAATACGGTTATCCACAGAATCAGGGGATAACGCAGGAAAG | | | |
| | | AACATGTGAGCAAAAGGCCAGCAAAAGGCCAGGAACCGTAAAAAGG | | | |
| | | CCGCGTTGCTGGCGTTTTTCCATAGGCTCCGCCCCCCTGACGAGCATC | | | |
| | | ACAAAAATCGACGCTCAAGTCAGAGGTGGCGAAACCCGACAGGACT | | | |
| | | ATAAAGATACCAGGCGTTTCCCCCTGGAAGCTCCCTCGTGCGCTCTCC | | | |
| | | TGTTCCGACCCTGCCGCTTACCGGATACCTGTCCGCCTTTCTCCCTTCG | | | |
| | | GGAAGCGTGGCGCTTTCTCATAGCTCACGCTGTAGGTATCTCAGTTCG | | | |
| | | GTGTAGGTCGTTCGCTCCAAGCTGGGCTGTGTGCACGAACCCCCCGT | | | |
| | | TCAGCCCGACCGCTGCGCCTTATCCGGTAACTATCGTCTTGAGTCCAA | | | |
| | | CCCGGTAAGACACGACTTATCGCCACTGGCAGCAGCCACTGGTAACA | | | |
| | | GGATTAGCAGAGCGAGGTATGTAGGCGGTGCTACAGAGTTCTTGAA | | | |
| | | GTGGTGGCCTAACTACGGCTACACTAGAAGGACAGTATTTGGTATCT | | | |
| | | GCGCTCTGCTGAAGCCAGTTACCTTCGGAAAAAGAGTTGGTAGCTCT | | | |
| | | TGATCCGGCAAACAAACCACCGCTGGTAGCGGTGGTTTTTTTGTTTGC | | | |
| | | AAGCAGCAGATTACGCGCAGAAAAAAAGGATCTCAAGAAGATCCTTT | | | |
| | | GATCTTTTCTACGGGGTCTGACGCTCAGTGGAACGAAAACTCACGTTA | | | |
| | | AGGGATTTTGGTCATGAGATTATCAAAAAGGATCTTCACCTAGATCCT | | | |
| | | TTTAAATTAAAAATGAAGTTTTAAATCAATCTAAAGTATATATGAGTA | | | |
| | | AACTTGGTCTGACAGTTACCAATGCTTAATCAGTGAGGCACCTATCTC | | | |
| | | AGCGATCTGTCTATTTCGTTCATCCATAGTTGCCTGACTCCCCGTCGTG | | | |
| | | TAGATAACTACGATACGGGAGGGCTTACCATCTGGCCCCAGTGCTGC | | | |
| | | AATGATACCGCGAGACCCACGCTCACCGGCTCCAGATTTATCAGCAAT | | | |
| | | AAACCAGCCAGCCGGAAGGGCCGAGCGCAGAAGTGGTCCTGCAACT | | | |
| | | TTATCCGCCTCCATCCAGTCTATTAATTGTTGCCGGGAAGCTAGAGTA | | | |
| | | AGTAGTTCGCCAGTTAATAGTTTGCGCAACGTTGTTGCCATTGCTACA | | | |
| | | GGCATCGTGGTGTCACGCTCGTCGTTTGGTATGGCTTCATTCAGCTCC | | | |
| | | GGTTCCCAACGATCAAGGCGAGTTACATGATCCCCCATGTTGTGCAA | | | |
| | | AAAAGCGGTTAGCTCCTTCGGTCCTCCGATCGTTGTCAGAAGTAAGTT | | | |
| | | GGCCGCAGTGTTATCACTCATGGTTATGGCAGCACTGCATAATTCTCT | | | |
| | | TACTGTCATGCCATCCGTAAGATGCTTTTCTGTGACTGGTGAGTACTC | | | |
| | | AACCAAGTCATTCTGAGAATAGTGTATGCGGCGACCGAGTTGCTCTT | | | |
| | | GCCCGGCGTCAATACGGGATAATACCGCGCCACATAGCAGAACTTTA | | | |
| | | AAAGTGCTCATCATTGGAAAACGTTCTTCGGGGCGAAAACTCTCAAG | | | |
| | | GATCTTACCGCTGTTGAGATCCAGTTCGATGTAACCCACTCGTGCACC | | | |
| | | CAACTGATCTTCAGCATCTTTTACTTTCACCAGCGTTTCTGGGTGAGCA | | | |
| | | AAAACAGGAAGGCAAAATGCCGCAAAAAAGGGAATAAGGGCGACA | | | |
| | | CGGAAATGTTGAATACTCATACTCTTCCTTTTTCAATATTATTGAAGCA | | | |
| | | TTTATCAGGGTTATTGTCTCATGAGCGGATACATATTTGAATGTATTT | | | |
| | | AGAAAAATAAACAAATAGGGGTTCCGCGCACATTTCCCCGAAAAGTG | | | |
| | | CCACCTGACGTCTAAGAAACCATTATTATCATGACATTAACCTATAAA | | | |
| | | AATAGGCGTATCACGAGGCCCTTTCGTCTCGCGCGTTTCGGTGATGA | | | |
| | | CGGTGAAAACCTCTGACACATGCAGCTCCCGGAGACGGTCACAGCTT | | | |
| | | GTCTGTAAGCGGATGCCGGGAGCAGACAAGCCCGTCAGGGCGCGTC | | | |
| | | AGCGGGTGTTGGCGGGTGTCGGGGCTGGCTTAACTATGCGGCATCA | | | |
| | | GAGCAGATTGTACTGAGAGTGCACCATATGCGGTGTGAAATACCGCA | | | |
| | | CAGATGCGTAAGGAGAAAATACCGCATCAGGCGCCATTCGCCATTCA | | | |
| | | GGCTGCGCAACTGTTGGGAAGGGCGATCGGTGCGGGCCTCTTCGCT | | | |
| | | ATTACGCCAGCTGGCGAAAGGGGGATGTGCTGCAAGGCGATTAAGT | | | |
| | | TGGGTAACGCCAGGGTTTTCCCAGTCACGACGTTGTAAAACGACGGC | | | |
| | | CAGT | | | |
| TEMPLATE_ | PLV10062 | GAATTCGAGCTTGCATGCCTGCAGGTCGTTACATAACTTACGGTAAAT | 323 | N/A | |
| COMPACT_ | | GGCCCGCCTGGCTGACCGCCCAACGACCCCCGCCCATTGACGTCAAT | | | |
| pCMV_ | | AATGACGTATGTTCCCATAGTAACGCCAATAGGGACTTTCCATTGACG | | | |
| R-U5- | | TCAATGGGTGGAGTATTTACGGTAAACTGCCCACTTGGCAGTACATC | | | |
| MusD6_ | | AAGTGTATCATATGCCAAGTACGCCCCCTATTGACGTCAATGACGGTA | | | |
| EF1-GFPai- | | AATGGCCCGCCTGGCATTATGCCCAGTACATGACCTTATGGGACTTTC | | | |
| LTR_v1 | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGAGCTTTGATCAGAATGAA | | | |
| | | TTTGTCTTGGCTCCGTTTCTTCTTTCGCCCCGTCTAGATTCCTCTCTTAC | | | |
| | | AGCTCGAGTGGCCTTCTCAGTCGAACCGTTCACGTTGCGAGCTGCTG | | | |
| | | GCGGCCGCAACATTTTGGCGCCAGAACTGGGACCTGAAGAATGGCga | | | |
| | | acaaacgacccaacacccgtgcgttttattctgtctttttattgccgatcccccggccgcttta | | | |
| | | cttgtacagctcgtccatgccgagagtgatcccggcggcggtcacgaactccagcaggacc | | | |
| | | atgtgatcgcgcttctcgttggggtctttgctcagggcggactgggtgctcaggtaagtatca | | | |
| | | aggttacaagacaggtttaaggagaccaatagaaactgggcttgtcgagacagagaagac | | | |
| | | tcttgcgtttctgataggcacctattggtcttactgacatccactttgcctttctctccacaggt | | | |
| | | agtggttgtcgggcagcagcacggggccgtcgccgatgggggtgttctgctggtagtggtcg | | | |
| | | gcgagctgcacgctgccgtcctcgatgttgtggcggatcttgaagttcaccttgatgccgttc | | | |
| | | ttctgcttgtcggccatgatatagacgttgtggctgttgtagttgtactccagcttgtgcccca | | | |
| | | ggatgttgccgtcctccttgaagtcgatgcccttcagctcgatgcggttcaccagggtgtcgc | | | |
| | | cctcgaacttcacctcggcgcgggtcttgtagttgccgtcgtccttgaagaagatggtgcgct | | | |
| | | cctggacgtagccttcgggcatggcggacttgaagaagtcgtgctgcttcatgtggtcgggg | | | |
| | | tagcggctgaagcactgcacgccgtaggtcagggtggtcacgagggtgggccagggcacg | | | |
| | | ggcagcttgccggtggtgcagatgaacttcagggtcagcttgccgtaggtggcatcgccctc | | | |
| | | gccctcgccggacacgctgaacttgtggccgtttacgtcgccgtccagctcgaccaggatgg | | | |
| | | gcaccaccccggtgaacagctcctcgcccttgctcaccatggtggctttaccaacagtaccg | | | |
| | | gaatgccaagcttgggtcctgtgttctggcggcaaacccgttgcgaaaaagaacgttcacg | | | |
| | | gcgactactgcacttatatacggttctcccccaccctcgggaaaaaggcggagccagtaca | | | |
| | | cgacatcactttcccagtttaccccgcgccaccttctctaggcaccggatcaattgccgaccc | | | |
| | | ctccccccaacttctcggggactgtgggcgatgtgcgctctgcccTCAGGCCAGCTTTT | | | |
| | | TCTTTTTTTTTAATTTTGTTAATAAAAGGGAGGAGATGTAGTCTCCCCT | | | |
| | | CCCCCAGCCTGAAACCTGCTTGCTCAGGGGTGGAGCTTCCCGCTCATC | | | |
| | | GCTCTGCCACGCCCACTGCTGGAACCTGCGGAGCCACACACGTGCAC | | | |
| | | CTTTCTACTGGACCAGAGATTATTCGGCGGGAATCGGGTCCCCTCCCC | | | |
| | | CTTCCTTCATAACTAGTGTCCCAACAATAAAATTTGAGCTTTGATCAGA | | | |
| | | ATGAATTTGTCTTGGCTCCGTTTCTTCTTTCGCCCCGTCTAGATTCCTCT | | | |
| | | CTTACAGCTCGAGTGGCCTTCTCAGTCGAACCGTTCACGTTGCGAGCT | | | |
| | | GCTGGCGGCCGCAACAGGGGATCCAGACATGATAAGATACATTGAT | | | |
| | | GAGTTTGGACAAACCACAACTAGAATGCAGTGAAAAAAATGCTTTAT | | | |
| | | TTGTGAAATTTGTGATGCTATTGCTTTATTTGTAACCATTATAAGCTGC | | | |
| | | AATAAACAAGTTAACAACAACAATTGCATTCATTTTATGTTTCAGGTTC | | | |
| | | AGGGGGAGGTGTGGGAGGTTTTTTCGGATCCTCTAGAGTCGACCTGC | | | |
| | | AGGCATGCAAGCTTGGCGTAATCATGGTCATAGCTGTTTCCTGTGTG | | | |
| | | AAATTGTTATCCGCTCACAATTCCACACAACATACGAGCCGGAAGCAT | | | |
| | | AAAGTGTAAAGCCTGGGGTGCCTAATGAGTGAGCTAACTCACATTAA | | | |
| | | TTGCGTTGCGCTCACTGCCCGCTTTCCAGTCGGGAAACCTGTCGTGCC | | | |
| | | AGCTGCATTAATGAATCGGCCAACGCGCGGGGAGAGGCGGTTTGCG | | | |
| | | TATTGGGCGCTCTTCCGCTTCCTCGCTCACTGACTCGCTGCGCTCGGT | | | |
| | | CGTTCGGCTGCGGCGAGCGGTATCAGCTCACTCAAAGGCGGTAATAC | | | |
| | | GGTTATCCACAGAATCAGGGGATAACGCAGGAAAGAACATGTGAGC | | | |
| | | AAAAGGCCAGCAAAAGGCCAGGAACCGTAAAAAGGCCGCGTTGCTG | | | |
| | | GCGTTTTTCCATAGGCTCCGCCCCCCTGACGAGCATCACAAAAATCGA | | | |
| | | CGCTCAAGTCAGAGGTGGCGAAACCCGACAGGACTATAAAGATACC | | | |
| | | AGGCGTTTCCCCCTGGAAGCTCCCTCGTGCGCTCTCCTGTTCCGACCC | | | |
| | | TGCCGCTTACCGGATACCTGTCCGCCTTTCTCCCTTCGGGAAGCGTGG | | | |
| | | CGCTTTCTCATAGCTCACGCTGTAGGTATCTCAGTTCGGTGTAGGTCG | | | |
| | | TTCGCTCCAAGCTGGGCTGTGTGCACGAACCCCCCGTTCAGCCCGACC | | | |
| | | GCTGCGCCTTATCCGGTAACTATCGTCTTGAGTCCAACCCGGTAAGAC | | | |
| | | ACGACTTATCGCCACTGGCAGCAGCCACTGGTAACAGGATTAGCAGA | | | |
| | | GCGAGGTATGTAGGCGGTGCTACAGAGTTCTTGAAGTGGTGGCCTAA | | | |
| | | CTACGGCTACACTAGAAGGACAGTATTTGGTATCTGCGCTCTGCTGAA | | | |
| | | GCCAGTTACCTTCGGAAAAAGAGTTGGTAGCTCTTGATCCGGCAAAC | | | |
| | | AAACCACCGCTGGTAGCGGTGGTTTTTTTGTTTGCAAGCAGCAGATTA | | | |
| | | CGCGCAGAAAAAAAGGATCTCAAGAAGATCCTTTGATCTTTTCTACG | | | |
| | | GGGTCTGACGCTCAGTGGAACGAAAACTCACGTTAAGGGATTTTGGT | | | |
| | | CATGAGATTATCAAAAAGGATCTTCACCTAGATCCTTTTAAATTAAAA | | | |
| | | ATGAAGTTTTAAATCAATCTAAAGTATATATGAGTAAACTTGGTCTGA | | | |
| | | CAGTTACCAATGCTTAATCAGTGAGGCACCTATCTCAGCGATCTGTCT | | | |
| | | ATTTCGTTCATCCATAGTTGCCTGACTCCCCGTCGTGTAGATAACTAC | | | |
| | | GATACGGGAGGGCTTACCATCTGGCCCCAGTGCTGCAATGATACCGC | | | |
| | | GAGACCCACGCTCACCGGCTCCAGATTTATCAGCAATAAACCAGCCA | | | |
| | | GCCGGAAGGGCCGAGCGCAGAAGTGGTCCTGCAACTTTATCCGCCTC | | | |
| | | CATCCAGTCTATTAATTGTTGCCGGGAAGCTAGAGTAAGTAGTTCGCC | | | |
| | | AGTTAATAGTTTGCGCAACGTTGTTGCCATTGCTACAGGCATCGTGGT | | | |
| | | GTCACGCTCGTCGTTTGGTATGGCTTCATTCAGCTCCGGTTCCCAACG | | | |
| | | ATCAAGGCGAGTTACATGATCCCCCATGTTGTGCAAAAAAGCGGTTA | | | |
| | | GCTCCTTCGGTCCTCCGATCGTTGTCAGAAGTAAGTTGGCCGCAGTGT | | | |
| | | TATCACTCATGGTTATGGCAGCACTGCATAATTCTCTTACTGTCATGCC | | | |
| | | ATCCGTAAGATGCTTTTCTGTGACTGGTGAGTACTCAACCAAGTCATT | | | |
| | | CTGAGAATAGTGTATGCGGCGACCGAGTTGCTCTTGCCCGGCGTCAA | | | |
| | | TACGGGATAATACCGCGCCACATAGCAGAACTTTAAAAGTGCTCATC | | | |
| | | ATTGGAAAACGTTCTTCGGGGCGAAAACTCTCAAGGATCTTACCGCT | | | |
| | | GTTGAGATCCAGTTCGATGTAACCCACTCGTGCACCCAACTGATCTTC | | | |
| | | AGCATCTTTTACTTTCACCAGCGTTTCTGGGTGAGCAAAAACAGGAAG | | | |
| | | GCAAAATGCCGCAAAAAAGGGAATAAGGGCGACACGGAAATGTTGA | | | |
| | | ATACTCATACTCTTCCTTTTTCAATATTATTGAAGCATTTATCAGGGTT | | | |
| | | ATTGTCTCATGAGCGGATACATATTTGAATGTATTTAGAAAAATAAAC | | | |
| | | AAATAGGGGTTCCGCGCACATTTCCCCGAAAAGTGCCACCTGACGTC | | | |
| | | TAAGAAACCATTATTATCATGACATTAACCTATAAAAATAGGCGTATC | | | |
| | | ACGAGGCCCTTTCGTCTCGCGCGTTTCGGTGATGACGGTGAAAACCT | | | |
| | | CTGACACATGCAGCTCCCGGAGACGGTCACAGCTTGTCTGTAAGCGG | | | |
| | | ATGCCGGGAGCAGACAAGCCCGTCAGGGCGCGTCAGCGGGTGTTGG | | | |
| | | CGGGTGTCGGGGCTGGCTTAACTATGCGGCATCAGAGCAGATTGTAC | | | |
| | | TGAGAGTGCACCATATGCGGTGTGAAATACCGCACAGATGCGTAAG | | | |
| | | GAGAAAATACCGCATCAGGCGCCATTCGCCATTCAGGCTGCGCAACT | | | |
| | | GTTGGGAAGGGCGATCGGTGCGGGCCTCTTCGCTATTACGCCAGCTG | | | |
| | | GCGAAAGGGGGATGTGCTGCAAGGCGATTAAGTTGGGTAACGCCAG | | | |
| | | GGTTTTCCCAGTCACGACGTTGTAAAACGACGGCCAGT | | | |
| TEMPLATE_ | PLV10063 | GAATTCGAGCTTGCATGCCTGCAGGTCGTTACATAACTTACGGTAAAT | 324 | N/A | |
| COMPACT_ | | GGCCCGCCTGGCTGACCGCCCAACGACCCCCGCCCATTGACGTCAAT | | | |
| pCMV_ | | AATGACGTATGTTCCCATAGTAACGCCAATAGGGACTTTCCATTGACG | | | |
| R-U5- | | TCAATGGGTGGAGTATTTACGGTAAACTGCCCACTTGGCAGTACATC | | | |
| MusD6_ | | AAGTGTATCATATGCCAAGTACGCCCCCTATTGACGTCAATGACGGTA | | | |
| EF1-GFPai- | | AATGGCCCGCCTGGCATTATGCCCAGTACATGACCTTATGGGACTTTC | | | |
| LTR_v2 | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGCTTTGATCAGAATGAATTT | | | |
| | | GTCTTGGCTCCGTTTCTTCTTTCGCCCCGTCTAGATTCCTCTCTTACAGC | | | |
| | | TCGAGTGGCCTTCTCAGTCGAACCGTTCACGTTGCGAGCTGCTGGCG | | | |
| | | GCCGCAACATTTTGGCGCCAGAACTGGGACCTGAAGAATGGCgaacaa | | | |
| | | acgacccaacacccgtgcgttttattctgtctttttattgccgatcccccggccgctttacttgt | | | |
| | | acagctcgtccatgccgagagtgatcccggcggcggtcacgaactccagcaggaccatgtg | | | |
| | | atcgcgcttctcgttggggtctttgctcagggcggactgggtgctcaggtaagtatcaaggtt | | | |
| | | acaagacaggtttaaggagaccaatagaaactgggcttgtcgagacagagaagactcttg | | | |
| | | cgtttctgataggcacctattggtcttactgacatccactttgcctttctctccacaggtagtgg | | | |
| | | ttgtcgggcagcagcacggggccgtcgccgatgggggtgttctgctggtagtggtcggcga | | | |
| | | gctgcacgctgccgtcctcgatgttgtggcggatcttgaagttcaccttgatgccgttcttctg | | | |
| | | cttgtcggccatgatatagacgttgtggctgttgtagttgtactccagcttgtgccccaggatg | | | |
| | | ttgccgtcctccttgaagtcgatgcccttcagctcgatgcggttcaccagggtgtcgccctcg | | | |
| | | aacttcacctcggcgcgggtcttgtagttgccgtcgtccttgaagaagatggtgcgctcctgg | | | |
| | | acgtagccttcgggcatggcggacttgaagaagtcgtgctgcttcatgtggtcggggtagcg | | | |
| | | gctgaagcactgcacgccgtaggtcagggtggtcacgagggtgggccagggcacgggcag | | | |
| | | cttgccggtggtgcagatgaacttcagggtcagcttgccgtaggtggcatcgccctcgccct | | | |
| | | cgccggacacgctgaacttgtggccgtttacgtcgccgtccagctcgaccaggatgggcacc | | | |
| | | accccggtgaacagctcctcgcccttgctcaccatggtggctttaccaacagtaccggaatg | | | |
| | | ccaagcttgggtcctgtgttctggcggcaaacccgttgcgaaaaagaacgttcacggcgact | | | |
| | | actgcacttatatacggttctcccccaccctcgggaaaaaggcggagccagtacacgacat | | | |
| | | cactttcccagtttaccccgcgccaccttctctaggcaccggatcaattgccgacccctcccc | | | |
| | | ccaacttctcggggactgtgggcgatgtgcgctctgcccTCAGGCCAGCTTTTTCTTT | | | |
| | | TTTTTTAATTTTGTTAATAAAAGGGAGGAGATGTAGTCTCCCCTCCCCC | | | |
| | | AGCCTGAAACCTGCTTGCTCAGGGGTGGAGCTTCCCGCTCATCGCTCT | | | |
| | | GCCACGCCCACTGCTGGAACCTGCGGAGCCACACACGTGCACCTTTCT | | | |
| | | ACTGGACCAGAGATTATTCGGCGGGAATCGGGTCCCCTCCCCCTTCCT | | | |
| | | TCATAACTAGTGTCCCAACAATAAAATTTGAGCTTTGATCAGAATGAA | | | |
| | | TTTGTCTTGGCTCCGTTTCTTCTTTCGCCCCGTCTAGATTCCTCTCTTAC | | | |
| | | AGCTCGAGTGGCCTTCTCAGTCGAACCGTTCACGTTGCGAGCTGCTG | | | |
| | | GCGGCCGCAACAGGGGATCCAGACATGATAAGATACATTGATGAGTT | | | |
| | | TGGACAAACCACAACTAGAATGCAGTGAAAAAAATGCTTTATTTGTG | | | |
| | | AAATTTGTGATGCTATTGCTTTATTTGTAACCATTATAAGCTGCAATAA | | | |
| | | ACAAGTTAACAACAACAATTGCATTCATTTTATGTTTCAGGTTCAGGG | | | |
| | | GGAGGTGTGGGAGGTTTTTTCGGATCCTCTAGAGTCGACCTGCAGGC | | | |
| | | ATGCAAGCTTGGCGTAATCATGGTCATAGCTGTTTCCTGTGTGAAATT | | | |
| | | GTTATCCGCTCACAATTCCACACAACATACGAGCCGGAAGCATAAAG | | | |
| | | TGTAAAGCCTGGGGTGCCTAATGAGTGAGCTAACTCACATTAATTGC | | | |
| | | GTTGCGCTCACTGCCCGCTTTCCAGTCGGGAAACCTGTCGTGCCAGCT | | | |
| | | GCATTAATGAATCGGCCAACGCGCGGGGAGAGGCGGTTTGCGTATT | | | |
| | | GGGCGCTCTTCCGCTTCCTCGCTCACTGACTCGCTGCGCTCGGTCGTT | | | |
| | | CGGCTGCGGCGAGCGGTATCAGCTCACTCAAAGGCGGTAATACGGTT | | | |
| | | ATCCACAGAATCAGGGGATAACGCAGGAAAGAACATGTGAGCAAAA | | | |
| | | GGCCAGCAAAAGGCCAGGAACCGTAAAAAGGCCGCGTTGCTGGCGT | | | |
| | | TTTTCCATAGGCTCCGCCCCCCTGACGAGCATCACAAAAATCGACGCT | | | |
| | | CAAGTCAGAGGTGGCGAAACCCGACAGGACTATAAAGATACCAGGC | | | |
| | | GTTTCCCCCTGGAAGCTCCCTCGTGCGCTCTCCTGTTCCGACCCTGCC | | | |
| | | GCTTACCGGATACCTGTCCGCCTTTCTCCCTTCGGGAAGCGTGGCGCT | | | |
| | | TTCTCATAGCTCACGCTGTAGGTATCTCAGTTCGGTGTAGGTCGTTCG | | | |
| | | CTCCAAGCTGGGCTGTGTGCACGAACCCCCCGTTCAGCCCGACCGCT | | | |
| | | GCGCCTTATCCGGTAACTATCGTCTTGAGTCCAACCCGGTAAGACACG | | | |
| | | ACTTATCGCCACTGGCAGCAGCCACTGGTAACAGGATTAGCAGAGCG | | | |
| | | AGGTATGTAGGCGGTGCTACAGAGTTCTTGAAGTGGTGGCCTAACTA | | | |
| | | CGGCTACACTAGAAGGACAGTATTTGGTATCTGCGCTCTGCTGAAGC | | | |
| | | CAGTTACCTTCGGAAAAAGAGTTGGTAGCTCTTGATCCGGCAAACAA | | | |
| | | ACCACCGCTGGTAGCGGTGGTTTTTTTGTTTGCAAGCAGCAGATTACG | | | |
| | | CGCAGAAAAAAAGGATCTCAAGAAGATCCTTTGATCTTTTCTACGGG | | | |
| | | GTCTGACGCTCAGTGGAACGAAAACTCACGTTAAGGGATTTTGGTCA | | | |
| | | TGAGATTATCAAAAAGGATCTTCACCTAGATCCTTTTAAATTAAAAAT | | | |
| | | GAAGTTTTAAATCAATCTAAAGTATATATGAGTAAACTTGGTCTGACA | | | |
| | | GTTACCAATGCTTAATCAGTGAGGCACCTATCTCAGCGATCTGTCTAT | | | |
| | | TTCGTTCATCCATAGTTGCCTGACTCCCCGTCGTGTAGATAACTACGA | | | |
| | | TACGGGAGGGCTTACCATCTGGCCCCAGTGCTGCAATGATACCGCGA | | | |
| | | GACCCACGCTCACCGGCTCCAGATTTATCAGCAATAAACCAGCCAGCC | | | |
| | | GGAAGGGCCGAGCGCAGAAGTGGTCCTGCAACTTTATCCGCCTCCAT | | | |
| | | CCAGTCTATTAATTGTTGCCGGGAAGCTAGAGTAAGTAGTTCGCCAG | | | |
| | | TTAATAGTTTGCGCAACGTTGTTGCCATTGCTACAGGCATCGTGGTGT | | | |
| | | CACGCTCGTCGTTTGGTATGGCTTCATTCAGCTCCGGTTCCCAACGAT | | | |
| | | CAAGGCGAGTTACATGATCCCCCATGTTGTGCAAAAAAGCGGTTAGC | | | |
| | | TCCTTCGGTCCTCCGATCGTTGTCAGAAGTAAGTTGGCCGCAGTGTTA | | | |
| | | TCACTCATGGTTATGGCAGCACTGCATAATTCTCTTACTGTCATGCCAT | | | |
| | | CCGTAAGATGCTTTTCTGTGACTGGTGAGTACTCAACCAAGTCATTCT | | | |
| | | GAGAATAGTGTATGCGGCGACCGAGTTGCTCTTGCCCGGCGTCAATA | | | |
| | | CGGGATAATACCGCGCCACATAGCAGAACTTTAAAAGTGCTCATCATT | | | |
| | | GGAAAACGTTCTTCGGGGCGAAAACTCTCAAGGATCTTACCGCTGTT | | | |
| | | GAGATCCAGTTCGATGTAACCCACTCGTGCACCCAACTGATCTTCAGC | | | |
| | | ATCTTTTACTTTCACCAGCGTTTCTGGGTGAGCAAAAACAGGAAGGCA | | | |
| | | AAATGCCGCAAAAAAGGGAATAAGGGCGACACGGAAATGTTGAATA | | | |
| | | CTCATACTCTTCCTTTTTCAATATTATTGAAGCATTTATCAGGGTTATT | | | |
| | | GTCTCATGAGCGGATACATATTTGAATGTATTTAGAAAAATAAACAAA | | | |
| | | TAGGGGTTCCGCGCACATTTCCCCGAAAAGTGCCACCTGACGTCTAA | | | |
| | | GAAACCATTATTATCATGACATTAACCTATAAAAATAGGCGTATCACG | | | |
| | | AGGCCCTTTCGTCTCGCGCGTTTCGGTGATGACGGTGAAAACCTCTG | | | |
| | | ACACATGCAGCTCCCGGAGACGGTCACAGCTTGTCTGTAAGCGGATG | | | |
| | | CCGGGAGCAGACAAGCCCGTCAGGGCGCGTCAGCGGGTGTTGGCGG | | | |
| | | GTGTCGGGGCTGGCTTAACTATGCGGCATCAGAGCAGATTGTACTGA | | | |
| | | GAGTGCACCATATGCGGTGTGAAATACCGCACAGATGCGTAAGGAG | | | |
| | | AAAATACCGCATCAGGCGCCATTCGCCATTCAGGCTGCGCAACTGTT | | | |
| | | GGGAAGGGCGATCGGTGCGGGCCTCTTCGCTATTACGCCAGCTGGC | | | |
| | | GAAAGGGGGATGTGCTGCAAGGCGATTAAGTTGGGTAACGCCAGGG | | | |
| | | TTTTCCCAGTCACGACGTTGTAAAACGACGGCCAGT | | | |
| TEMPLATE_ | PLV10064 | GAATTCGAGCTTGCATGCCTGCAGGTCGTTACATAACTTACGGTAAAT | 325 | N/A | |
| COMPACT_ | | GGCCCGCCTGGCTGACCGCCCAACGACCCCCGCCCATTGACGTCAAT | | | |
| pCMVR- | | AATGACGTATGTTCCCATAGTAACGCCAATAGGGACTTTCCATTGACG | | | |
| U5- | | TCAATGGGTGGAGTATTTACGGTAAACTGCCCACTTGGCAGTACATC | | | |
| MusD6_ | | AAGTGTATCATATGCCAAGTACGCCCCCTATTGACGTCAATGACGGTA | | | |
| EF1-GFPai- | | AATGGCCCGCCTGGCATTATGCCCAGTACATGACCTTATGGGACTTTC | | | |
| LTR_v3 | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGGGTAGGCGTGTACGGTGGGAGGTCTATA | | | |
| | | TAAGCAGAGCTGAGCTTTGATCAGAATGAATTTGTCTTGGCTCCGTTT | | | |
| | | CTTCTTTCGCCCCGTCTAGATTCCTCTCTTACAGCTCGAGTGGCCTTCT | | | |
| | | CAGTCGAACCGTTCACGTTGCGAGCTGCTGGCGGCCGCAACATTTTG | | | |
| | | GCGCCAGAACTGGGACCTGAAGAATGGCgaacaaacgacccaacacccgtg | | | |
| | | cgttttattctgtctttttattgccgatcccccggccgctttacttgtacagctcgtccatgccg | | | |
| | | agagtgatcccggcggcggtcacgaactccagcaggaccatgtgatcgcgcttctcgttgg | | | |
| | | ggtctttgctcagggcggactgggtgctcaggtaagtatcaaggttacaagacaggtttaag | | | |
| | | gagaccaatagaaactgggcttgtcgagacagagaagactcttgcgtttctgataggcacc | | | |
| | | tattggtcttactgacatccactttgcctttctctccacaggtagtggttgtcgggcagcagca | | | |
| | | cggggccgtcgccgatgggggtgttctgctggtagtggtcggcgagctgcacgctgccgtcc | | | |
| | | tcgatgttgtggcggatcttgaagttcaccttgatgccgttcttctgcttgtcggccatgatat | | | |
| | | agacgttgtggctgttgtagttgtactccagcttgtgccccaggatgttgccgtcctccttgaa | | | |
| | | gtcgatgcccttcagctcgatgcggttcaccagggtgtcgccctcgaacttcacctcggcgcg | | | |
| | | ggtcttgtagttgccgtcgtccttgaagaagatggtgcgctcctggacgtagccttcgggcat | | | |
| | | ggcggacttgaagaagtcgtgctgcttcatgtggtcggggtagcggctgaagcactgcacg | | | |
| | | ccgtaggtcagggtggtcacgagggtgggccagggcacgggcagcttgccggtggtgcag | | | |
| | | atgaacttcagggtcagcttgccgtaggtggcatcgccctcgccctcgccggacacgctgaa | | | |
| | | cttgtggccgtttacgtcgccgtccagctcgaccaggatgggcaccaccccggtgaacagct | | | |
| | | cctcgcccttgctcaccatggtggctttaccaacagtaccggaatgccaagcttgggtcctgt | | | |
| | | gttctggcggcaaacccgttgcgaaaaagaacgttcacggcgactactgcacttatatacg | | | |
| | | gttctcccccaccctcgggaaaaaggcggagccagtacacgacatcactttcccagtttacc | | | |
| | | ccgcgccaccttctctaggcaccggatcaattgccgacccctccccccaacttctcggggact | | | |
| | | gtgggcgatgtgcgctctgcccTCAGGCCAGCTTTTTCTTTTTTTTTAATTTTGT | | | |
| | | TAATAAAAGGGAGGAGATGTAGTCTCCCCTCCCCCAGCCTGAAACCT | | | |
| | | GCTTGCTCAGGGGTGGAGCTTCCCGCTCATCGCTCTGCCACGCCCACT | | | |
| | | GCTGGAACCTGCGGAGCCACACACGTGCACCTTTCTACTGGACCAGA | | | |
| | | GATTATTCGGCGGGAATCGGGTCCCCTCCCCCTTCCTTCATAACTAGT | | | |
| | | GTCCCAACAATAAAATTTGAGCTTTGATCAGAATGAATTTGTCTTGGC | | | |
| | | TCCGTTTCTTCTTTCGCCCCGTCTAGATTCCTCTCTTACAGCTCGAGTG | | | |
| | | GCCTTCTCAGTCGAACCGTTCACGTTGCGAGCTGCTGGCGGCCGCAA | | | |
| | | CAGGGGATCCAGACATGATAAGATACATTGATGAGTTTGGACAAACC | | | |
| | | ACAACTAGAATGCAGTGAAAAAAATGCTTTATTTGTGAAATTTGTGAT | | | |
| | | GCTATTGCTTTATTTGTAACCATTATAAGCTGCAATAAACAAGTTAAC | | | |
| | | AACAACAATTGCATTCATTTTATGTTTCAGGTTCAGGGGGAGGTGTG | | | |
| | | GGAGGTTTTTTCGGATCCTCTAGAGTCGACCTGCAGGCATGCAAGCT | | | |
| | | TGGCGTAATCATGGTCATAGCTGTTTCCTGTGTGAAATTGTTATCCGC | | | |
| | | TCACAATTCCACACAACATACGAGCCGGAAGCATAAAGTGTAAAGCC | | | |
| | | TGGGGTGCCTAATGAGTGAGCTAACTCACATTAATTGCGTTGCGCTCA | | | |
| | | CTGCCCGCTTTCCAGTCGGGAAACCTGTCGTGCCAGCTGCATTAATGA | | | |
| | | ATCGGCCAACGCGCGGGGAGAGGCGGTTTGCGTATTGGGCGCTCTT | | | |
| | | CCGCTTCCTCGCTCACTGACTCGCTGCGCTCGGTCGTTCGGCTGCGGC | | | |
| | | GAGCGGTATCAGCTCACTCAAAGGCGGTAATACGGTTATCCACAGAA | | | |
| | | TCAGGGGATAACGCAGGAAAGAACATGTGAGCAAAAGGCCAGCAAA | | | |
| | | AGGCCAGGAACCGTAAAAAGGCCGCGTTGCTGGCGTTTTTCCATAGG | | | |
| | | CTCCGCCCCCCTGACGAGCATCACAAAAATCGACGCTCAAGTCAGAG | | | |
| | | GTGGCGAAACCCGACAGGACTATAAAGATACCAGGCGTTTCCCCCTG | | | |
| | | GAAGCTCCCTCGTGCGCTCTCCTGTTCCGACCCTGCCGCTTACCGGAT | | | |
| | | ACCTGTCCGCCTTTCTCCCTTCGGGAAGCGTGGCGCTTTCTCATAGCT | | | |
| | | CACGCTGTAGGTATCTCAGTTCGGTGTAGGTCGTTCGCTCCAAGCTG | | | |
| | | GGCTGTGTGCACGAACCCCCCGTTCAGCCCGACCGCTGCGCCTTATCC | | | |
| | | GGTAACTATCGTCTTGAGTCCAACCCGGTAAGACACGACTTATCGCCA | | | |
| | | CTGGCAGCAGCCACTGGTAACAGGATTAGCAGAGCGAGGTATGTAG | | | |
| | | GCGGTGCTACAGAGTTCTTGAAGTGGTGGCCTAACTACGGCTACACT | | | |
| | | AGAAGGACAGTATTTGGTATCTGCGCTCTGCTGAAGCCAGTTACCTTC | | | |
| | | GGAAAAAGAGTTGGTAGCTCTTGATCCGGCAAACAAACCACCGCTGG | | | |
| | | TAGCGGTGGTTTTTTTGTTTGCAAGCAGCAGATTACGCGCAGAAAAA | | | |
| | | AAGGATCTCAAGAAGATCCTTTGATCTTTTCTACGGGGTCTGACGCTC | | | |
| | | AGTGGAACGAAAACTCACGTTAAGGGATTTTGGTCATGAGATTATCA | | | |
| | | AAAAGGATCTTCACCTAGATCCTTTTAAATTAAAAATGAAGTTTTAAA | | | |
| | | TCAATCTAAAGTATATATGAGTAAACTTGGTCTGACAGTTACCAATGC | | | |
| | | TTAATCAGTGAGGCACCTATCTCAGCGATCTGTCTATTTCGTTCATCCA | | | |
| | | TAGTTGCCTGACTCCCCGTCGTGTAGATAACTACGATACGGGAGGGC | | | |
| | | TTACCATCTGGCCCCAGTGCTGCAATGATACCGCGAGACCCACGCTCA | | | |
| | | CCGGCTCCAGATTTATCAGCAATAAACCAGCCAGCCGGAAGGGCCGA | | | |
| | | GCGCAGAAGTGGTCCTGCAACTTTATCCGCCTCCATCCAGTCTATTAA | | | |
| | | TTGTTGCCGGGAAGCTAGAGTAAGTAGTTCGCCAGTTAATAGTTTGC | | | |
| | | GCAACGTTGTTGCCATTGCTACAGGCATCGTGGTGTCACGCTCGTCGT | | | |
| | | TTGGTATGGCTTCATTCAGCTCCGGTTCCCAACGATCAAGGCGAGTTA | | | |
| | | CATGATCCCCCATGTTGTGCAAAAAAGCGGTTAGCTCCTTCGGTCCTC | | | |
| | | CGATCGTTGTCAGAAGTAAGTTGGCCGCAGTGTTATCACTCATGGTTA | | | |
| | | TGGCAGCACTGCATAATTCTCTTACTGTCATGCCATCCGTAAGATGCT | | | |
| | | TTTCTGTGACTGGTGAGTACTCAACCAAGTCATTCTGAGAATAGTGTA | | | |
| | | TGCGGCGACCGAGTTGCTCTTGCCCGGCGTCAATACGGGATAATACC | | | |
| | | GCGCCACATAGCAGAACTTTAAAAGTGCTCATCATTGGAAAACGTTCT | | | |
| | | TCGGGGCGAAAACTCTCAAGGATCTTACCGCTGTTGAGATCCAGTTC | | | |
| | | GATGTAACCCACTCGTGCACCCAACTGATCTTCAGCATCTTTTACTTTC | | | |
| | | ACCAGCGTTTCTGGGTGAGCAAAAACAGGAAGGCAAAATGCCGCAA | | | |
| | | AAAAGGGAATAAGGGCGACACGGAAATGTTGAATACTCATACTCTTC | | | |
| | | CTTTTTCAATATTATTGAAGCATTTATCAGGGTTATTGTCTCATGAGCG | | | |
| | | GATACATATTTGAATGTATTTAGAAAAATAAACAAATAGGGGTTCCG | | | |
| | | CGCACATTTCCCCGAAAAGTGCCACCTGACGTCTAAGAAACCATTATT | | | |
| | | ATCATGACATTAACCTATAAAAATAGGCGTATCACGAGGCCCTTTCGT | | | |
| | | CTCGCGCGTTTCGGTGATGACGGTGAAAACCTCTGACACATGCAGCT | | | |
| | | CCCGGAGACGGTCACAGCTTGTCTGTAAGCGGATGCCGGGAGCAGA | | | |
| | | CAAGCCCGTCAGGGCGCGTCAGCGGGTGTTGGCGGGTGTCGGGGCT | | | |
| | | GGCTTAACTATGCGGCATCAGAGCAGATTGTACTGAGAGTGCACCAT | | | |
| | | ATGCGGTGTGAAATACCGCACAGATGCGTAAGGAGAAAATACCGCA | | | |
| | | TCAGGCGCCATTCGCCATTCAGGCTGCGCAACTGTTGGGAAGGGCGA | | | |
| | | TCGGTGCGGGCCTCTTCGCTATTACGCCAGCTGGCGAAAGGGGGATG | | | |
| | | TGCTGCAAGGCGATTAAGTTGGGTAACGCCAGGGTTTTCCCAGTCAC | | | |
| | | GACGTTGTAAAACGACGGCCAGT | | | |
| TEMPLATE_ | PLV10072 | GAATTCGAGCTTGCATGCCTGCAGGTCGTTACATAACTTACGGTAAAT | 326 | N/A | |
| pCMV_ | | GGCCCGCCTGGCTGACCGCCCAACGACCCCCGCCCATTGACGTCAAT | | | |
| R-U5- | | AATGACGTATGTTCCCATAGTAACGCCAATAGGGACTTTCCATTGACG | | | |
| MusD6_ | | TCAATGGGTGGAGTATTTACGGTAAACTGCCCACTTGGCAGTACATC | | | |
| EF1-GFPai- | | AAGTGTATCATATGCCAAGTACGCCCCCTATTGACGTCAATGACGGTA | | | |
| LTR_v1 | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGAGCTTTGATCAGAATGAA | | | |
| | | TTTGTCTTGGCTCCGTTTCTTCTTTCGCCCCGTCTAGATTCCTCTCTTAC | | | |
| | | AGCTCGAGTGGCCTTCTCAGTCGAACCGTTCACGTTGCGAGCTGCTG | | | |
| | | GCGGCCGCAACATTTTGGCGCCAGAACTGGGACCTGAAGAATGGCA | | | |
| | | GAGAGATGCTAAGAGGAACGCTGCTTTGGAGCTCCACAGGAAAGGA | | | |
| | | TCTTCGTATCGGACATCGGAGCAACGGACAGGTACACATGCTAGCGC | | | |
| | | TAGCTTAAAATTTCAGTTTTGTAAAGTGTTGCTGAGGATGTGGTAGG | | | |
| | | ATACGAATTAAGCTTGAATCAGTGCTAACCCAACGCTGGTTCTGCTTG | | | |
| | | GGTCAGCAGCGTGTTAATCGGAACTAGAAACGGAAACAGGCAGGTT | | | |
| | | AGCCGCAGCTTTTTAGGAAGCTGCTTAGGTGGAAGAAGAAAGGGTTT | | | |
| | | AAAGTCATGGATCAGGCGGTTGCCCATAGTTTTCAGGAGTTGTTTCA | | | |
| | | GGCCAGAGGAGTAAGGCTTGAAGTACAATTAGTAAAAAATTTTTTAG | | | |
| | | GTAAGATAGATAGCTGTTGCCCATGGTTCAAGGAAGAAGAAACACTA | | | |
| | | GATTGTGGAACCTGGGAGAAAGTTGGTGAGGCCTTAAAAATCACTCA | | | |
| | | GGCAGATAATTTTACCCTAGAATTTGGGGTAGAGGCTCAGTTTGTGTT | | | |
| | | TTTTCACGAGATGAAGATGGAGCACGGTGGCTGCCAGAGAGATTAAT | | | |
| | | TCGTCAGACGAACACAGATTCTGACTCTTCTGGTAAGTATCATTCTAA | | | |
| | | AGACTAAAATTCCTTTTGTGCTTAAAATTCAGCTGAGAGCAACAGCTC | | | |
| | | TCAAAGCTGTTCTCCAGCTACTCTCTGAGCCAGCTCCCGACAGGAGGC | | | |
| | | CGGAGACTAGCCTCAGCTTTACAATTTGCATTTgaacaaacgacccaacacc | | | |
| | | cgtgcgttttattctgtctttttattgccgatcccccggccgctttacttgtacagctcgtccatg | | | |
| | | ccgagagtgatcccggcggcggtcacgaactccagcaggaccatgtgatcgcgcttctcgtt | | | |
| | | ggggtctttgctcagggcggactgggtgctcaggtaagtatcaaggttacaagacaggttta | | | |
| | | aggagaccaatagaaactgggcttgtcgagacagagaagactcttgcgtttctgataggca | | | |
| | | cctattggtcttactgacatccactttgcctttctctccacaggtagtggttgtcgggcagcag | | | |
| | | cacggggccgtcgccgatgggggtgttctgctggtagtggtcggcgagctgcacgctgccgt | | | |
| | | cctcgatgttgtggcggatcttgaagttcaccttgatgccgttcttctgcttgtcggccatgat | | | |
| | | atagacgttgtggctgttgtagttgtactccagcttgtgccccaggatgttgccgtcctccttg | | | |
| | | aagtcgatgcccttcagctcgatgcggttcaccagggtgtcgccctcgaacttcacctcggc | | | |
| | | gcgggtcttgtagttgccgtcgtccttgaagaagatggtgcgctcctggacgtagccttcggg | | | |
| | | catggcggacttgaagaagtcgtgctgcttcatgtggtcggggtagcggctgaagcactgc | | | |
| | | acgccgtaggtcagggtggtcacgagggtgggccagggcacgggcagcttgccggtggtg | | | |
| | | cagatgaacttcagggtcagcttgccgtaggtggcatcgccctcgccctcgccggacacgct | | | |
| | | gaacttgtggccgtttacgtcgccgtccagctcgaccaggatgggcaccaccccggtgaac | | | |
| | | agctcctcgcccttgctcaccatggtggctttaccaacagtaccggaatgccaagcttgggtc | | | |
| | | ctgtgttctggcggcaaacccgttgcgaaaaagaacgttcacggcgactactgcacttatat | | | |
| | | acggttctcccccaccctcgggaaaaaggcggagccagtacacgacatcactttcccagttt | | | |
| | | accccgcgccaccttctctaggcaccggatcaattgccgacccctccccccaacttctcggg | | | |
| | | gactgtgggcgatgtgcgctctgcccAAATAAAGTACCTAGACTTCCCCGAAAA | | | |
| | | AAGTTCTGCTTTTCTACTTTCTCACTGTCTTTCAAGATTTTGTCTTTCAA | | | |
| | | GCAGGTAAATCAACATTCTCGAGGCGGACCAGCGGATGTGCATCCCC | | | |
| | | GCCCCCCTAGAGCACTCAGGTGGCAGCTGTTATCCCCAGTCTCAGGA | | | |
| | | CATTCCAGCATGTGGCCTTCAGTCTGAGTTAAAAATTAGGTTTACCCA | | | |
| | | GAGGACTAGAATAGTAGATATTTCTATATTAATAAAGATTGGTTTTTA | | | |
| | | TTTTGATAGACAGGCTTAGCCCCTTAGCTGACCTCTGGCTTTTCACCCT | | | |
| | | TGCTGTTACTGCAAGGTGTCTTTAGCTCAATAAGGCTGTGGAAAAAA | | | |
| | | ACAGGGATGAGGAGGAACGGCTCCCAGCTCCTATTTTAGCCACAAAT | | | |
| | | CGTGGTGTTACTAACGACATAATTCTTGCTTAGGCTTTGCTAAATCTG | | | |
| | | AGGTTGATAATTCTCCTTTAGGAGCTGCACAGCGCTCAGAACTGTGCA | | | |
| | | TACTGATTTGTGATGGTACAAATTCAGTATGGGCATCGCTTGGTGCA | | | |
| | | GATGGAGGTACTGCAAGGAAAGGTCCCAGCTTGACCATTTCTGAGTT | | | |
| | | TCCTGTGAGATAAACCCGGTTTGAAAGAGGTTGGTACCAAATTATAT | | | |
| | | ATCCCTCGGCTCTACCTCGCCTCCCCAAAAGGTACCAGAGCCACAGGT | | | |
| | | GTGGATTTTAACAGAATCCACGGGAGGAATCGGGTCCATGTCCACCC | | | |
| | | AAGCCAAGGTTAAAAGCCCACTCATCTACGGATGAGAAAATCATTTG | | | |
| | | ATCACCTCAGTTAAGCGCTGCCTTATTTTAACTTAATTAATAGGGGGG | | | |
| | | AGAGAGATTGGAGACTTACTATTGAAAGGGCAAGCCCTTCACTGCCT | | | |
| | | CCCACCCAAATAAAAAAGCCAATTGGCCTTGTACTACAGAGCTGGCC | | | |
| | | GGACCCCTTATCCCTGTTACCCACCAATCATCCAAAAATGCGGAGGAA | | | |
| | | TATCAACTTAGTGTTATTCTTATTATAGTGTATTTCACACTTGTTCAGTC | | | |
| | | AAACTTAGCCAGAGTTCCAACGCCCTACTTAAAATTCAACTAGAAAGT | | | |
| | | TACCTACCAAGTACTAATTAGCATTATAAAGTCAGAGCCTACAGCTCC | | | |
| | | AGGCTTTTCAGTTAGTTGTTTACTAAGATAAGAAAAGACAGTCTTAGC | | | |
| | | CAGATACAGTTTACCATAATAAAAGTTAAAGAATCCCAGGGAAGCAA | | | |
| | | GTTTTTTCTTTTAGCCCTAGATTCCAGGCAGAACTATTGAGCATAGAT | | | |
| | | AATTTTTCCCCCTCAGGCCAGCTTTTTCTTTTTTTTTAATTTTGTTAATA | | | |
| | | AAAGGGAGGAGATGTAGTCTCCCCTCCCCCAGCCTGAAACCTGCTTG | | | |
| | | CTCAGGGGTGGAGCTTCCCGCTCATCGCTCTGCCACGCCCACTGCTG | | | |
| | | GAACCTGCGGAGCCACACACGTGCACCTTTCTACTGGACCAGAGATT | | | |
| | | ATTCGGCGGGAATCGGGTCCCCTCCCCCTTCCTTCATAACTAGTGTCC | | | |
| | | CAACAATAAAATTTGAGCTTTGATCAGAATGAATTTGTCTTGGCTCCG | | | |
| | | TTTCTTCTTTCGCCCCGTCTAGATTCCTCTCTTACAGCTCGAGTGGCCT | | | |
| | | TCTCAGTCGAACCGTTCACGTTGCGAGCTGCTGGCGGCCGCAACAGG | | | |
| | | GGATCCAGACATGATAAGATACATTGATGAGTTTGGACAAACCACAA | | | |
| | | CTAGAATGCAGTGAAAAAAATGCTTTATTTGTGAAATTTGTGATGCTA | | | |
| | | TTGCTTTATTTGTAACCATTATAAGCTGCAATAAACAAGTTAACAACA | | | |
| | | ACAATTGCATTCATTTTATGTTTCAGGTTCAGGGGGAGGTGTGGGAG | | | |
| | | GTTTTTTCGGATCCTCTAGAGTCGACCTGCAGGCATGCAAGCTTGGCG | | | |
| | | TAATCATGGTCATAGCTGTTTCCTGTGTGAAATTGTTATCCGCTCACAA | | | |
| | | TTCCACACAACATACGAGCCGGAAGCATAAAGTGTAAAGCCTGGGGT | | | |
| | | GCCTAATGAGTGAGCTAACTCACATTAATTGCGTTGCGCTCACTGCCC | | | |
| | | GCTTTCCAGTCGGGAAACCTGTCGTGCCAGCTGCATTAATGAATCGG | | | |
| | | CCAACGCGCGGGGAGAGGCGGTTTGCGTATTGGGCGCTCTTCCGCTT | | | |
| | | CCTCGCTCACTGACTCGCTGCGCTCGGTCGTTCGGCTGCGGCGAGCG | | | |
| | | GTATCAGCTCACTCAAAGGCGGTAATACGGTTATCCACAGAATCAGG | | | |
| | | GGATAACGCAGGAAAGAACATGTGAGCAAAAGGCCAGCAAAAGGCC | | | |
| | | AGGAACCGTAAAAAGGCCGCGTTGCTGGCGTTTTTCCATAGGCTCCG | | | |
| | | CCCCCCTGACGAGCATCACAAAAATCGACGCTCAAGTCAGAGGTGGC | | | |
| | | GAAACCCGACAGGACTATAAAGATACCAGGCGTTTCCCCCTGGAAGC | | | |
| | | TCCCTCGTGCGCTCTCCTGTTCCGACCCTGCCGCTTACCGGATACCTGT | | | |
| | | CCGCCTTTCTCCCTTCGGGAAGCGTGGCGCTTTCTCATAGCTCACGCT | | | |
| | | GTAGGTATCTCAGTTCGGTGTAGGTCGTTCGCTCCAAGCTGGGCTGT | | | |
| | | GTGCACGAACCCCCCGTTCAGCCCGACCGCTGCGCCTTATCCGGTAAC | | | |
| | | TATCGTCTTGAGTCCAACCCGGTAAGACACGACTTATCGCCACTGGCA | | | |
| | | GCAGCCACTGGTAACAGGATTAGCAGAGCGAGGTATGTAGGCGGTG | | | |
| | | CTACAGAGTTCTTGAAGTGGTGGCCTAACTACGGCTACACTAGAAGG | | | |
| | | ACAGTATTTGGTATCTGCGCTCTGCTGAAGCCAGTTACCTTCGGAAAA | | | |
| | | AGAGTTGGTAGCTCTTGATCCGGCAAACAAACCACCGCTGGTAGCGG | | | |
| | | TGGTTTTTTTGTTTGCAAGCAGCAGATTACGCGCAGAAAAAAAGGAT | | | |
| | | CTCAAGAAGATCCTTTGATCTTTTCTACGGGGTCTGACGCTCAGTGGA | | | |
| | | ACGAAAACTCACGTTAAGGGATTTTGGTCATGAGATTATCAAAAAGG | | | |
| | | ATCTTCACCTAGATCCTTTTAAATTAAAAATGAAGTTTTAAATCAATCT | | | |
| | | AAAGTATATATGAGTAAACTTGGTCTGACAGTTACCAATGCTTAATCA | | | |
| | | GTGAGGCACCTATCTCAGCGATCTGTCTATTTCGTTCATCCATAGTTG | | | |
| | | CCTGACTCCCCGTCGTGTAGATAACTACGATACGGGAGGGCTTACCA | | | |
| | | TCTGGCCCCAGTGCTGCAATGATACCGCGAGACCCACGCTCACCGGC | | | |
| | | TCCAGATTTATCAGCAATAAACCAGCCAGCCGGAAGGGCCGAGCGCA | | | |
| | | GAAGTGGTCCTGCAACTTTATCCGCCTCCATCCAGTCTATTAATTGTTG | | | |
| | | CCGGGAAGCTAGAGTAAGTAGTTCGCCAGTTAATAGTTTGCGCAACG | | | |
| | | TTGTTGCCATTGCTACAGGCATCGTGGTGTCACGCTCGTCGTTTGGTA | | | |
| | | TGGCTTCATTCAGCTCCGGTTCCCAACGATCAAGGCGAGTTACATGAT | | | |
| | | CCCCCATGTTGTGCAAAAAAGCGGTTAGCTCCTTCGGTCCTCCGATCG | | | |
| | | TTGTCAGAAGTAAGTTGGCCGCAGTGTTATCACTCATGGTTATGGCA | | | |
| | | GCACTGCATAATTCTCTTACTGTCATGCCATCCGTAAGATGCTTTTCTG | | | |
| | | TGACTGGTGAGTACTCAACCAAGTCATTCTGAGAATAGTGTATGCGG | | | |
| | | CGACCGAGTTGCTCTTGCCCGGCGTCAATACGGGATAATACCGCGCC | | | |
| | | ACATAGCAGAACTTTAAAAGTGCTCATCATTGGAAAACGTTCTTCGGG | | | |
| | | GCGAAAACTCTCAAGGATCTTACCGCTGTTGAGATCCAGTTCGATGTA | | | |
| | | ACCCACTCGTGCACCCAACTGATCTTCAGCATCTTTTACTTTCACCAGC | | | |
| | | GTTTCTGGGTGAGCAAAAACAGGAAGGCAAAATGCCGCAAAAAAGG | | | |
| | | GAATAAGGGCGACACGGAAATGTTGAATACTCATACTCTTCCTTTTTC | | | |
| | | AATATTATTGAAGCATTTATCAGGGTTATTGTCTCATGAGCGGATACA | | | |
| | | TATTTGAATGTATTTAGAAAAATAAACAAATAGGGGTTCCGCGCACAT | | | |
| | | TTCCCCGAAAAGTGCCACCTGACGTCTAAGAAACCATTATTATCATGA | | | |
| | | CATTAACCTATAAAAATAGGCGTATCACGAGGCCCTTTCGTCTCGCGC | | | |
| | | GTTTCGGTGATGACGGTGAAAACCTCTGACACATGCAGCTCCCGGAG | | | |
| | | ACGGTCACAGCTTGTCTGTAAGCGGATGCCGGGAGCAGACAAGCCC | | | |
| | | GTCAGGGCGCGTCAGCGGGTGTTGGCGGGTGTCGGGGCTGGCTTAA | | | |
| | | CTATGCGGCATCAGAGCAGATTGTACTGAGAGTGCACCATATGCGGT | | | |
| | | GTGAAATACCGCACAGATGCGTAAGGAGAAAATACCGCATCAGGCG | | | |
| | | CCATTCGCCATTCAGGCTGCGCAACTGTTGGGAAGGGCGATCGGTGC | | | |
| | | GGGCCTCTTCGCTATTACGCCAGCTGGCGAAAGGGGGATGTGCTGCA | | | |
| | | AGGCGATTAAGTTGGGTAACGCCAGGGTTTTCCCAGTCACGACGTTG | | | |
| | | TAAAACGACGGCCAGT | | | |
| TEMPLATE_ | PLV10073 | GAATTCGAGCTTGCATGCCTGCAGGTCGTTACATAACTTACGGTAAAT | 327 | N/A | |
| pCMV_ | | GGCCCGCCTGGCTGACCGCCCAACGACCCCCGCCCATTGACGTCAAT | | | |
| R-U5- | | AATGACGTATGTTCCCATAGTAACGCCAATAGGGACTTTCCATTGACG | | | |
| MusD6_ | | TCAATGGGTGGAGTATTTACGGTAAACTGCCCACTTGGCAGTACATC | | | |
| EF1-GFPai- | | AAGTGTATCATATGCCAAGTACGCCCCCTATTGACGTCAATGACGGTA | | | |
| LTR_v2 | | AATGGCCCGCCTGGCATTATGCCCAGTACATGACCTTATGGGACTTTC | | | |
| | | CTACTTGGCAGTACATCTACGTATTAGTCATCGCTATTACCATGGTGA | | | |
| | | TGCGGTTTTGGCAGTACATCAATGGGCGTGGATAGCGGTTTGACTCA | | | |
| | | CGGGGATTTCCAAGTCTCCACCCCATTGACGTCAATGGGAGTTTGTTT | | | |
| | | TGGCACCAAAATCAACGGGACTTTCCAAAATGTCGTAACAACTCCGCC | | | |
| | | CCATTGACGCAAATGGGCGGTAGGCGTGTACGGTGGGAGGTCTATA | | | |
| | | TAAGCAGAGCTCGTTTAGTGAACCGTCAGCTTTGATCAGAATGAATTT | | | |
| | | GTCTTGGCTCCGTTTCTTCTTTCGCCCCGTCTAGATTCCTCTCTTACAGC | | | |
| | | TCGAGTGGCCTTCTCAGTCGAACCGTTCACGTTGCGAGCTGCTGGCG | | | |
| | | GCCGCAACATTTTGGCGCCAGAACTGGGACCTGAAGAATGGCAGAG | | | |
| | | AGATGCTAAGAGGAACGCTGCTTTGGAGCTCCACAGGAAAGGATCTT | | | |
| | | CGTATCGGACATCGGAGCAACGGACAGGTACACATGCTAGCGCTAGC | | | |
| | | TTAAAATTTCAGTTTTGTAAAGTGTTGCTGAGGATGTGGTAGGATAC | | | |
| | | GAATTAAGCTTGAATCAGTGCTAACCCAACGCTGGTTCTGCTTGGGTC | | | |
| | | AGCAGCGTGTTAATCGGAACTAGAAACGGAAACAGGCAGGTTAGCC | | | |
| | | GCAGCTTTTTAGGAAGCTGCTTAGGTGGAAGAAGAAAGGGTTTAAA | | | |
| | | GTCATGGATCAGGCGGTTGCCCATAGTTTTCAGGAGTTGTTTCAGGC | | | |
| | | CAGAGGAGTAAGGCTTGAAGTACAATTAGTAAAAAATTTTTTAGGTA | | | |
| | | AGATAGATAGCTGTTGCCCATGGTTCAAGGAAGAAGAAACACTAGAT | | | |
| | | TGTGGAACCTGGGAGAAAGTTGGTGAGGCCTTAAAAATCACTCAGGC | | | |
| | | AGATAATTTTACCCTAGAATTTGGGGTAGAGGCTCAGTTTGTGTTTTT | | | |
| | | TCACGAGATGAAGATGGAGCACGGTGGCTGCCAGAGAGATTAATTC | | | |
| | | GTCAGACGAACACAGATTCTGACTCTTCTGGTAAGTATCATTCTAAAG | | | |
| | | ACTAAAATTCCTTTTGTGCTTAAAATTCAGCTGAGAGCAACAGCTCTC | | | |
| | | AAAGCTGTTCTCCAGCTACTCTCTGAGCCAGCTCCCGACAGGAGGCC | | | |
| | | GGAGACTAGCCTCAGCTTTACAATTTGCATTTgaacaaacgacccaacaccc | | | |
| | | gtgcgttttattctgtctttttattgccgatcccccggccgctttacttgtacagctcgtccatgc | | | |
| | | cgagagtgatcccggcggcggtcacgaactccagcaggaccatgtgatcgcgcttctcgtt | | | |
| | | ggggtctttgctcagggcggactgggtgctcaggtaagtatcaaggttacaagacaggttta | | | |
| | | aggagaccaatagaaactgggcttgtcgagacagagaagactcttgcgtttctgataggca | | | |
| | | cctattggtcttactgacatccactttgcctttctctccacaggtagtggttgtcgggcagcag | | | |
| | | cacggggccgtcgccgatgggggtgttctgctggtagtggtcggcgagctgcacgctgccgt | | | |
| | | cctcgatgttgtggcggatcttgaagttcaccttgatgccgttcttctgcttgtcggccatgat | | | |
| | | atagacgttgtggctgttgtagttgtactccagcttgtgccccaggatgttgccgtcctccttg | | | |
| | | aagtcgatgcccttcagctcgatgcggttcaccagggtgtcgccctcgaacttcacctcggc | | | |
| | | gcgggtcttgtagttgccgtcgtccttgaagaagatggtgcgctcctggacgtagccttcggg | | | |
| | | catggcggacttgaagaagtcgtgctgcttcatgtggtcggggtagcggctgaagcactgc | | | |
| | | acgccgtaggtcagggtggtcacgagggtgggccagggcacgggcagcttgccggtggtg | | | |
| | | cagatgaacttcagggtcagcttgccgtaggtggcatcgccctcgccctcgccggacacgct | | | |
| | | gaacttgtggccgtttacgtcgccgtccagctcgaccaggatgggcaccaccccggtgaac | | | |
| | | agctcctcgcccttgctcaccatggtggctttaccaacagtaccggaatgccaagcttgggtc | | | |
| | | ctgtgttctggcggcaaacccgttgcgaaaaagaacgttcacggcgactactgcacttatat | | | |
| | | acggttctcccccaccctcgggaaaaaggcggagccagtacacgacatcactttcccagttt | | | |
| | | accccgcgccaccttctctaggcaccggatcaattgccgacccctccccccaacttctcggg | | | |
| | | gactgtgggcgatgtgcgctctgcccAAATAAAGTACCTAGACTTCCCCGAAAA | | | |
| | | AAGTTCTGCTTTTCTACTTTCTCACTGTCTTTCAAGATTTTGTCTTTCAA | | | |
| | | GCAGGTAAATCAACATTCTCGAGGCGGACCAGCGGATGTGCATCCCC | | | |
| | | GCCCCCCTAGAGCACTCAGGTGGCAGCTGTTATCCCCAGTCTCAGGA | | | |
| | | CATTCCAGCATGTGGCCTTCAGTCTGAGTTAAAAATTAGGTTTACCCA | | | |
| | | GAGGACTAGAATAGTAGATATTTCTATATTAATAAAGATTGGTTTTTA | | | |
| | | TTTTGATAGACAGGCTTAGCCCCTTAGCTGACCTCTGGCTTTTCACCCT | | | |
| | | TGCTGTTACTGCAAGGTGTCTTTAGCTCAATAAGGCTGTGGAAAAAA | | | |
| | | ACAGGGATGAGGAGGAACGGCTCCCAGCTCCTATTTTAGCCACAAAT | | | |
| | | CGTGGTGTTACTAACGACATAATTCTTGCTTAGGCTTTGCTAAATCTG | | | |
| | | AGGTTGATAATTCTCCTTTAGGAGCTGCACAGCGCTCAGAACTGTGCA | | | |
| | | TACTGATTTGTGATGGTACAAATTCAGTATGGGCATCGCTTGGTGCA | | | |
| | | GATGGAGGTACTGCAAGGAAAGGTCCCAGCTTGACCATTTCTGAGTT | | | |
| | | TCCTGTGAGATAAACCCGGTTTGAAAGAGGTTGGTACCAAATTATAT | | | |
| | | ATCCCTCGGCTCTACCTCGCCTCCCCAAAAGGTACCAGAGCCACAGGT | | | |
| | | GTGGATTTTAACAGAATCCACGGGAGGAATCGGGTCCATGTCCACCC | | | |
| | | AAGCCAAGGTTAAAAGCCCACTCATCTACGGATGAGAAAATCATTTG | | | |
| | | ATCACCTCAGTTAAGCGCTGCCTTATTTTAACTTAATTAATAGGGGGG | | | |
| | | AGAGAGATTGGAGACTTACTATTGAAAGGGCAAGCCCTTCACTGCCT | | | |
| | | CCCACCCAAATAAAAAAGCCAATTGGCCTTGTACTACAGAGCTGGCC | | | |
| | | GGACCCCTTATCCCTGTTACCCACCAATCATCCAAAAATGCGGAGGAA | | | |
| | | TATCAACTTAGTGTTATTCTTATTATAGTGTATTTCACACTTGTTCAGTC | | | |
| | | AAACTTAGCCAGAGTTCCAACGCCCTACTTAAAATTCAACTAGAAAGT | | | |
| | | TACCTACCAAGTACTAATTAGCATTATAAAGTCAGAGCCTACAGCTCC | | | |
| | | AGGCTTTTCAGTTAGTTGTTTACTAAGATAAGAAAAGACAGTCTTAGC | | | |
| | | CAGATACAGTTTACCATAATAAAAGTTAAAGAATCCCAGGGAAGCAA | | | |
| | | GTTTTTTCTTTTAGCCCTAGATTCCAGGCAGAACTATTGAGCATAGAT | | | |
| | | AATTTTTCCCCCTCAGGCCAGCTTTTTCTTTTTTTTTAATTTTGTTAATA | | | |
| | | AAAGGGAGGAGATGTAGTCTCCCCTCCCCCAGCCTGAAACCTGCTTG | | | |
| | | CTCAGGGGTGGAGCTTCCCGCTCATCGCTCTGCCACGCCCACTGCTG | | | |
| | | GAACCTGCGGAGCCACACACGTGCACCTTTCTACTGGACCAGAGATT | | | |
| | | ATTCGGCGGGAATCGGGTCCCCTCCCCCTTCCTTCATAACTAGTGTCC | | | |
| | | CAACAATAAAATTTGAGCTTTGATCAGAATGAATTTGTCTTGGCTCCG | | | |
| | | TTTCTTCTTTCGCCCCGTCTAGATTCCTCTCTTACAGCTCGAGTGGCCT | | | |
| | | TCTCAGTCGAACCGTTCACGTTGCGAGCTGCTGGCGGCCGCAACAGG | | | |
| | | GGATCCAGACATGATAAGATACATTGATGAGTTTGGACAAACCACAA | | | |
| | | CTAGAATGCAGTGAAAAAAATGCTTTATTTGTGAAATTTGTGATGCTA | | | |
| | | TTGCTTTATTTGTAACCATTATAAGCTGCAATAAACAAGTTAACAACA | | | |
| | | ACAATTGCATTCATTTTATGTTTCAGGTTCAGGGGGAGGTGTGGGAG | | | |
| | | GTTTTTTCGGATCCTCTAGAGTCGACCTGCAGGCATGCAAGCTTGGCG | | | |
| | | TAATCATGGTCATAGCTGTTTCCTGTGTGAAATTGTTATCCGCTCACAA | | | |
| | | TTCCACACAACATACGAGCCGGAAGCATAAAGTGTAAAGCCTGGGGT | | | |
| | | GCCTAATGAGTGAGCTAACTCACATTAATTGCGTTGCGCTCACTGCCC | | | |
| | | GCTTTCCAGTCGGGAAACCTGTCGTGCCAGCTGCATTAATGAATCGG | | | |
| | | CCAACGCGCGGGGAGAGGCGGTTTGCGTATTGGGCGCTCTTCCGCTT | | | |
| | | CCTCGCTCACTGACTCGCTGCGCTCGGTCGTTCGGCTGCGGCGAGCG | | | |
| | | GTATCAGCTCACTCAAAGGCGGTAATACGGTTATCCACAGAATCAGG | | | |
| | | GGATAACGCAGGAAAGAACATGTGAGCAAAAGGCCAGCAAAAGGCC | | | |
| | | AGGAACCGTAAAAAGGCCGCGTTGCTGGCGTTTTTCCATAGGCTCCG | | | |
| | | CCCCCCTGACGAGCATCACAAAAATCGACGCTCAAGTCAGAGGTGGC | | | |
| | | GAAACCCGACAGGACTATAAAGATACCAGGCGTTTCCCCCTGGAAGC | | | |
| | | TCCCTCGTGCGCTCTCCTGTTCCGACCCTGCCGCTTACCGGATACCTGT | | | |
| | | CCGCCTTTCTCCCTTCGGGAAGCGTGGCGCTTTCTCATAGCTCACGCT | | | |
| | | GTAGGTATCTCAGTTCGGTGTAGGTCGTTCGCTCCAAGCTGGGCTGT | | | |
| | | GTGCACGAACCCCCCGTTCAGCCCGACCGCTGCGCCTTATCCGGTAAC | | | |
| | | TATCGTCTTGAGTCCAACCCGGTAAGACACGACTTATCGCCACTGGCA | | | |
| | | GCAGCCACTGGTAACAGGATTAGCAGAGCGAGGTATGTAGGCGGTG | | | |
| | | CTACAGAGTTCTTGAAGTGGTGGCCTAACTACGGCTACACTAGAAGG | | | |
| | | ACAGTATTTGGTATCTGCGCTCTGCTGAAGCCAGTTACCTTCGGAAAA | | | |
| | | AGAGTTGGTAGCTCTTGATCCGGCAAACAAACCACCGCTGGTAGCGG | | | |
| | | TGGTTTTTTTGTTTGCAAGCAGCAGATTACGCGCAGAAAAAAAGGAT | | | |
| | | CTCAAGAAGATCCTTTGATCTTTTCTACGGGGTCTGACGCTCAGTGGA | | | |
| | | ACGAAAACTCACGTTAAGGGATTTTGGTCATGAGATTATCAAAAAGG | | | |
| | | ATCTTCACCTAGATCCTTTTAAATTAAAAATGAAGTTTTAAATCAATCT | | | |
| | | AAAGTATATATGAGTAAACTTGGTCTGACAGTTACCAATGCTTAATCA | | | |
| | | GTGAGGCACCTATCTCAGCGATCTGTCTATTTCGTTCATCCATAGTTG | | | |
| | | CCTGACTCCCCGTCGTGTAGATAACTACGATACGGGAGGGCTTACCA | | | |
| | | TCTGGCCCCAGTGCTGCAATGATACCGCGAGACCCACGCTCACCGGC | | | |
| | | TCCAGATTTATCAGCAATAAACCAGCCAGCCGGAAGGGCCGAGCGCA | | | |
| | | GAAGTGGTCCTGCAACTTTATCCGCCTCCATCCAGTCTATTAATTGTTG | | | |
| | | CCGGGAAGCTAGAGTAAGTAGTTCGCCAGTTAATAGTTTGCGCAACG | | | |
| | | TTGTTGCCATTGCTACAGGCATCGTGGTGTCACGCTCGTCGTTTGGTA | | | |
| | | TGGCTTCATTCAGCTCCGGTTCCCAACGATCAAGGCGAGTTACATGAT | | | |
| | | CCCCCATGTTGTGCAAAAAAGCGGTTAGCTCCTTCGGTCCTCCGATCG | | | |

| | | | | |
|---|---|---|---|---|
| | | TTGTCAGAAGTAAGTTGGCCGCAGTGTTATCACTCATGGTTATGGCA | | |
| | | GCACTGCATAATTCTCTTACTGTCATGCCATCCGTAAGATGCTTTTCTG | | |
| | | TGACTGGTGAGTACTCAACCAAGTCATTCTGAGAATAGTGTATGCGG | | |
| | | CGACCGAGTTGCTCTTGCCCGGCGTCAATACGGGATAATACCGCGCC | | |
| | | ACATAGCAGAACTTTAAAAGTGCTCATCATTGGAAAACGTTCTTCGGG | | |
| | | GCGAAAACTCTCAAGGATCTTACCGCTGTTGAGATCCAGTTCGATGTA | | |
| | | ACCCACTCGTGCACCCAACTGATCTTCAGCATCTTTTACTTTCACCAGC | | |
| | | GTTTCTGGGTGAGCAAAAACAGGAAGGCAAAATGCCGCAAAAAAGG | | |
| | | GAATAAGGGCGACACGGAAATGTTGAATACTCATACTCTTCCTTTTTC | | |
| | | AATATTATTGAAGCATTTATCAGGGTTATTGTCTCATGAGCGGATACA | | |
| | | TATTTGAATGTATTTAGAAAAATAAACAAATAGGGGTTCCGCGCACAT | | |
| | | TTCCCCGAAAAGTGCCACCTGACGTCTAAGAAACCATTATTATCATGA | | |
| | | CATTAACCTATAAAAATAGGCGTATCACGAGGCCCTTTCGTCTCGCGC | | |
| | | GTTTCGGTGATGACGGTGAAAACCTCTGACACATGCAGCTCCCGGAG | | |
| | | ACGGTCACAGCTTGTCTGTAAGCGGATGCCGGGAGCAGACAAGCCC | | |
| | | GTCAGGGCGCGTCAGCGGGTGTTGGCGGGTGTCGGGGCTGGCTTAA | | |
| | | CTATGCGGCATCAGAGCAGATTGTACTGAGAGTGCACCATATGCGGT | | |
| | | GTGAAATACCGCACAGATGCGTAAGGAGAAAATACCGCATCAGGCG | | |
| | | CCATTCGCCATTCAGGCTGCGCAACTGTTGGGAAGGGCGATCGGTGC | | |
| | | GGGCCTCTTCGCTATTACGCCAGCTGGCGAAAGGGGGATGTGCTGCA | | |
| | | AGGCGATTAAGTTGGGTAACGCCAGGGTTTTCCCAGTCACGACGTTG | | |
| | | TAAAACGACGGCCAGT | | |
| TEMPLATE_ | PLV10074 | GAATTCGAGCTTGCATGCCTGCAGGTCGTTACATAACTTACGGTAAAT | 328 | N/A |
| pCMV_ | | GGCCCGCCTGGCTGACCGCCCAACGACCCCCGCCCATTGACGTCAAT | | |
| R-U5- | | AATGACGTATGTTCCCATAGTAACGCCAATAGGGACTTTCCATTGACG | | |
| MusD6_ | | TCAATGGGTGGAGTATTTACGGTAAACTGCCCACTTGGCAGTACATC | | |
| EF1-GFPai- | | AAGTGTATCATATGCCAAGTACGCCCCCTATTGACGTCAATGACGGTA | | |
| LTR_v3 | | AATGGCCCGCCTGGCATTATGCCCAGTACATGACCTTATGGGACTTTC | | |
| | | CTACTTGGCAGTACATCTACGTATTAGTCATCGCTATTACCATGGTGA | | |
| | | TGCGGTTTTGGCAGTACATCAATGGGCGTGGATAGCGGTTTGACTCA | | |
| | | CGGGGATTTCCAAGTCTCCACCCCATTGACGTCAATGGGAGTTTGTTT | | |
| | | TGGCACCAAAATCAACGGGACTTTCCAAAATGTCGTAACAACTCCGCC | | |
| | | CCATTGACGCAAATGGGCGGTAGGCGTGTACGGTGGGAGGTCTATA | | |
| | | TAAGCAGAGCTGAGCTTTGATCAGAATGAATTTGTCTTGGCTCCGTTT | | |
| | | CTTCTTTCGCCCCGTCTAGATTCCTCTCTTACAGCTCGAGTGGCCTTCT | | |
| | | CAGTCGAACCGTTCACGTTGCGAGCTGCTGGCGGCCGCAACATTTTG | | |
| | | GCGCCAGAACTGGGACCTGAAGAATGGCAGAGAGATGCTAAGAGGA | | |
| | | ACGCTGCTTTGGAGCTCCACAGGAAAGGATCTTCGTATCGGACATCG | | |
| | | GAGCAACGGACAGGTACACATGCTAGCGCTAGCTTAAAATTTCAGTT | | |
| | | TTGTAAAGTGTTGCTGAGGATGTGGTAGGATACGAATTAAGCTTGAA | | |
| | | TCAGTGCTAACCCAACGCTGGTTCTGCTTGGGTCAGCAGCGTGTTAAT | | |
| | | CGGAACTAGAAACGGAAACAGGCAGGTTAGCCGCAGCTTTTTAGGA | | |
| | | AGCTGCTTAGGTGGAAGAAGAAAGGGTTTAAAGTCATGGATCAGGC | | |
| | | GGTTGCCCATAGTTTTCAGGAGTTGTTTCAGGCCAGAGGAGTAAGGC | | |
| | | TTGAAGTACAATTAGTAAAAAATTTTTTAGGTAAGATAGATAGCTGTT | | |
| | | GCCCATGGTTCAAGGAAGAAGAAACACTAGATTGTGGAACCTGGGA | | |
| | | GAAAGTTGGTGAGGCCTTAAAAATCACTCAGGCAGATAATTTTACCCT | | |
| | | AGAATTTGGGGTAGAGGCTCAGTTTGTGTTTTTTCACGAGATGAAGA | | |
| | | TGGAGCACGGTGGCTGCCAGAGAGATTAATTCGTCAGACGAACACA | | |
| | | GATTCTGACTCTTCTGGTAAGTATCATTCTAAAGACTAAAATTCCTTTT | | |
| | | GTGCTTAAAATTCAGCTGAGAGCAACAGCTCTCAAAGCTGTTCTCCAG | | |
| | | CTACTCTCTGAGCCAGCTCCCGACAGGAGGCCGGAGACTAGCCTCAG | | |
| | | CTTTACAATTTGCATTTgaacaaacgacccaacacccgtgcgttttattctgtcttttt | | |
| | | attgccgatcccccggccgctttacttgtacagctcgtccatgccgagagtgatcccggcgg | | |
| | | cggtcacgaactccagcaggaccatgtgatcgcgcttctcgttggggtctttgctcagggcg | | |
| | | gactgggtgctcaggtaagtatcaaggttacaagacaggtttaaggagaccaatagaaact | | |
| | | gggcttgtcgagacagagaagactcttgcgtttctgataggcacctattggtcttactgacat | | |
| | | ccactttgcctttctctccacaggtagtggttgtcgggcagcagcacggggccgtcgccgat | | |
| | | gggggtgttctgctggtagtggtcggcgagctgcacgctgccgtcctcgatgttgtggcggat | | |
| | | cttgaagttcaccttgatgccgttcttctgcttgtcggccatgatatagacgttgtggctgttgt | | |
| | | agttgtactccagcttgtgccccaggatgttgccgtcctccttgaagtcgatgcccttcagctc | | |
| | | gatgcggttcaccagggtgtcgccctcgaacttcacctcggcgcgggtcttgtagttgccgtc | | |
| | | gtccttgaagaagatggtgcgctcctggacgtagccttcgggcatggcggacttgaagaag | | |
| | | tcgtgctgcttcatgtggtcggggtagcggctgaagcactgcacgccgtaggtcagggtggt | | |
| | | cacgaggggggccagggcacgggcagcttgccggtggtgcagatgaacttcagggtcag | | |
| | | cttgccgtaggtggcatcgccctcgccctcgccggacacgctgaacttgtggccgtttacgtc | | |
| | | gccgtccagctcgaccaggatgggcaccaccccggtgaacagctcctcgcccttgctcacc | | |
| | | atggtggctttaccaacagtaccggaatgccaagcttgggtcctgtgttctggcggcaaacc | | |
| | | cgttgcgaaaaagaacgttcacggcgactactgcacttatatacggttctcccccaccctcg | | |
| | | ggaaaaaggcggagccagtacacgacatcactttcccagtttaccccgcgccaccttctcta | | |
| | | ggcaccggatcaattgccgacccctccccccaacttctcggggactgtgggcgatgtgcgct | | |
| | | ctgcccAAATAAAGTACCTAGACTTCCCCGAAAAAAGTTCTGCTTTTCTA | | |
| | | CTTTCTCACTGTCTTTCAAGATTTTGTCTTTCAAGCAGGTAAATCAACA | | |
| | | TTCTCGAGGCGGACCAGCGGATGTGCATCCCCGCCCCCCTAGAGCAC | | |
| | | TCAGGTGGCAGCTGTTATCCCCAGTCTCAGGACATTCCAGCATGTGGC | | |
| | | CTTCAGTCTGAGTTAAAAATTAGGTTTACCCAGAGGACTAGAATAGTA | | |
| | | GATATTTCTATATTAATAAAGATTGGTTTTTATTTTGATAGACAGGCTT | | |
| | | AGCCCCTTAGCTGACCTCTGGCTTTTCACCCTTGCTGTTACTGCAAGGT | | |
| | | GTCTTTAGCTCAATAAGGCTGTGGAAAAAAACAGGGATGAGGAGGA | | |
| | | ACGGCTCCCAGCTCCTATTTTAGCCACAAATCGTGGTGTTACTAACGA | | |
| | | CATAATTCTTGCTTAGGCTTTGCTAAATCTGAGGTTGATAATTCTCCTT | | |
| | | TAGGAGCTGCACAGCGCTCAGAACTGTGCATACTGATTTGTGATGGT | | |
| | | ACAAATTCAGTATGGGCATCGCTTGGTGCAGATGGAGGTACTGCAAG | | |
| | | GAAAGGTCCCAGCTTGACCATTTCTGAGTTTCCTGTGAGATAAACCCG | | |
| | | GTTTGAAAGAGGTTGGTACCAAATTATATATCCCTCGGCTCTACCTCG | | |
| | | CCTCCCCAAAAGGTACCAGAGCCACAGGTGTGGATTTTAACAGAATC | | |
| | | CACGGGAGGAATCGGGTCCATGTCCACCCAAGCCAAGGTTAAAAGCC | | |
| | | CACTCATCTACGGATGAGAAAATCATTTGATCACCTCAGTTAAGCGCT | | |
| | | GCCTTATTTTAACTTAATTAATAGGGGGGAGAGAGATTGGAGACTTA | | |
| | | CTATTGAAAGGGCAAGCCCTTCACTGCCTCCCACCCAAATAAAAAAGC | | |
| | | CAATTGGCCTTGTACTACAGAGCTGGCCGGACCCCTTATCCCTGTTAC | | |
| | | CCACCAATCATCCAAAAATGCGGAGGAATATCAACTTAGTGTTATTCT | | |
| | | TATTATAGTGTATTTCACACTTGTTCAGTCAAACTTAGCCAGAGTTCCA | | |
| | | ACGCCCTACTTAAAATTCAACTAGAAAGTTACCTACCAAGTACTAATT | | |
| | | AGCATTATAAAGTCAGAGCCTACAGCTCCAGGCTTTTCAGTTAGTTGT | | |
| | | TTACTAAGATAAGAAAAGACAGTCTTAGCCAGATACAGTTTACCATAA | | |
| | | TAAAAGTTAAAGAATCCCAGGGAAGCAAGTTTTTTCTTTTAGCCCTAG | | |
| | | ATTCCAGGCAGAACTATTGAGCATAGATAATTTTTCCCCCTCAGGCCA | | |
| | | GCTTTTTCTTTTTTTTTAATTTTGTTAATAAAAGGGAGGAGATGTAGTC | | |
| | | TCCCCTCCCCCAGCCTGAAACCTGCTTGCTCAGGGGTGGAGCTTCCCG | | |
| | | CTCATCGCTCTGCCACGCCCACTGCTGGAACCTGCGGAGCCACACAC | | |
| | | GTGCACCTTTCTACTGGACCAGAGATTATTCGGCGGGAATCGGGTCC | | |
| | | CCTCCCCCTTCCTTCATAACTAGTGTCCCAACAATAAAATTTGAGCTTT | | |
| | | GATCAGAATGAATTTGTCTTGGCTCCGTTTCTTCTTTCGCCCCGTCTAG | | |
| | | ATTCCTCTCTTACAGCTCGAGTGGCCTTCTCAGTCGAACCGTTCACGTT | | |
| | | GCGAGCTGCTGGCGGCCGCAACAGGGGATCCAGACATGATAAGATA | | |
| | | CATTGATGAGTTTGGACAAACCACAACTAGAATGCAGTGAAAAAAAT | | |
| | | GCTTTATTTGTGAAATTTGTGATGCTATTGCTTTATTTGTAACCATTAT | | |
| | | AAGCTGCAATAAACAAGTTAACAACAACAATTGCATTCATTTTATGTT | | |
| | | TCAGGTTCAGGGGGAGGTGTGGGAGGTTTTTTCGGATCCTCTAGAGT | | |
| | | CGACCTGCAGGCATGCAAGCTTGGCGTAATCATGGTCATAGCTGTTTC | | |
| | | CTGTGTGAAATTGTTATCCGCTCACAATTCCACACAACATACGAGCCG | | |
| | | GAAGCATAAAGTGTAAAGCCTGGGGTGCCTAATGAGTGAGCTAACTC | | |
| | | ACATTAATTGCGTTGCGCTCACTGCCCGCTTTCCAGTCGGGAAACCTG | | |
| | | TCGTGCCAGCTGCATTAATGAATCGGCCAACGCGCGGGGAGAGGCG | | |
| | | GTTTGCGTATTGGGCGCTCTTCCGCTTCCTCGCTCACTGACTCGCTGC | | |
| | | GCTCGGTCGTTCGGCTGCGGCGAGCGGTATCAGCTCACTCAAAGGCG | | |
| | | GTAATACGGTTATCCACAGAATCAGGGGATAACGCAGGAAAGAACA | | |
| | | TGTGAGCAAAAGGCCAGCAAAAGGCCAGGAACCGTAAAAAGGCCGC | | |
| | | GTTGCTGGCGTTTTTCCATAGGCTCCGCCCCCCTGACGAGCATCACAA | | |
| | | AAATCGACGCTCAAGTCAGAGGTGGCGAAACCCGACAGGACTATAA | | |
| | | AGATACCAGGCGTTTCCCCCTGGAAGCTCCCTCGTGCGCTCTCCTGTT | | |
| | | CCGACCCTGCCGCTTACCGGATACCTGTCCGCCTTTCTCCCTTCGGGA | | |
| | | AGCGTGGCGCTTTCTCATAGCTCACGCTGTAGGTATCTCAGTTCGGTG | | |
| | | TAGGTCGTTCGCTCCAAGCTGGGCTGTGTGCACGAACCCCCCGTTCA | | |
| | | GCCCGACCGCTGCGCCTTATCCGGTAACTATCGTCTTGAGTCCAACCC | | |
| | | GGTAAGACACGACTTATCGCCACTGGCAGCAGCCACTGGTAACAGGA | | |
| | | TTAGCAGAGCGAGGTATGTAGGCGGTGCTACAGAGTTCTTGAAGTG | | |
| | | GTGGCCTAACTACGGCTACACTAGAAGGACAGTATTTGGTATCTGCG | | |
| | | CTCTGCTGAAGCCAGTTACCTTCGGAAAAAGAGTTGGTAGCTCTTGAT | | |
| | | CCGGCAAACAAACCACCGCTGGTAGCGGTGGTTTTTTTGTTTGCAAGC | | |
| | | AGCAGATTACGCGCAGAAAAAAAGGATCTCAAGAAGATCCTTTGATC | | |
| | | TTTTCTACGGGGTCTGACGCTCAGTGGAACGAAAACTCACGTTAAGG | | |
| | | GATTTTGGTCATGAGATTATCAAAAAGGATCTTCACCTAGATCCTTTT | | |
| | | AAATTAAAAATGAAGTTTTAAATCAATCTAAAGTATATATGAGTAAAC | | |
| | | TTGGTCTGACAGTTACCAATGCTTAATCAGTGAGGCACCTATCTCAGC | | |
| | | GATCTGTCTATTTCGTTCATCCATAGTTGCCTGACTCCCCGTCGTGTAG | | |
| | | ATAACTACGATACGGGAGGGCTTACCATCTGGCCCCAGTGCTGCAAT | | |
| | | GATACCGCGAGACCCACGCTCACCGGCTCCAGATTTATCAGCAATAA | | |
| | | ACCAGCCAGCCGGAAGGGCCGAGCGCAGAAGTGGTCCTGCAACTTT | | |
| | | ATCCGCCTCCATCCAGTCTATTAATTGTTGCCGGGAAGCTAGAGTAAG | | |
| | | TAGTTCGCCAGTTAATAGTTTGCGCAACGTTGTTGCCATTGCTACAGG | | |
| | | CATCGTGGTGTCACGCTCGTCGTTTGGTATGGCTTCATTCAGCTCCGG | | |
| | | TTCCCAACGATCAAGGCGAGTTACATGATCCCCCATGTTGTGCAAAAA | | |
| | | AGCGGTTAGCTCCTTCGGTCCTCCGATCGTTGTCAGAAGTAAGTTGGC | | |
| | | CGCAGTGTTATCACTCATGGTTATGGCAGCACTGCATAATTCTCTTAC | | |
| | | TGTCATGCCATCCGTAAGATGCTTTTCTGTGACTGGTGAGTACTCAAC | | |
| | | CAAGTCATTCTGAGAATAGTGTATGCGGCGACCGAGTTGCTCTTGCCC | | |
| | | GGCGTCAATACGGGATAATACCGCGCCACATAGCAGAACTTTAAAAG | | |
| | | TGCTCATCATTGGAAAACGTTCTTCGGGGCGAAAACTCTCAAGGATCT | | |
| | | TACCGCTGTTGAGATCCAGTTCGATGTAACCCACTCGTGCACCCAACT | | |
| | | GATCTTCAGCATCTTTTACTTTCACCAGCGTTTCTGGGTGAGCAAAAA | | |
| | | CAGGAAGGCAAAATGCCGCAAAAAAGGGAATAAGGGCGACACGGA | | |
| | | AATGTTGAATACTCATACTCTTCCTTTTTCAATATTATTGAAGCATTTAT | | |
| | | CAGGGTTATTGTCTCATGAGCGGATACATATTTGAATGTATTTAGAAA | | |
| | | AATAAACAAATAGGGGTTCCGCGCACATTTCCCCGAAAAGTGCCACC | | |
| | | TGACGTCTAAGAAACCATTATTATCATGACATTAACCTATAAAAATAG | | |
| | | GCGTATCACGAGGCCCTTTCGTCTCGCGCGTTTCGGTGATGACGGTG | | |
| | | AAAACCTCTGACACATGCAGCTCCCGGAGACGGTCACAGCTTGTCTG | | |
| | | TAAGCGGATGCCGGGAGCAGACAAGCCCGTCAGGGCGCGTCAGCGG | | |
| | | GTGTTGGCGGGTGTCGGGGCTGGCTTAACTATGCGGCATCAGAGCA | | |
| | | GATTGTACTGAGAGTGCACCATATGCGGTGTGAAATACCGCACAGAT | | |
| | | GCGTAAGGAGAAAATACCGCATCAGGCGCCATTCGCCATTCAGGCTG | | |
| | | CGCAACTGTTGGGAAGGGCGATCGGTGCGGGCCTCTTCGCTATTACG | | |
| | | CCAGCTGGCGAAAGGGGGATGTGCTGCAAGGCGATTAAGTTGGGTA | | |
| | | ACGCCAGGGTTTTCCCAGTCACGACGTTGTAAAACGACGGCCAGT | | |

TABLE S3
ETnII LTR retrotransposon driver and template construct elements

| | | | SEQ | |
|---|---|---|---|---|
| | plasmid_ | | ID | protein_ |
| name | id | nucleic_acid_sequence | NO: | sequence |
| ETnII-B3 U3 | N/A | TGTAGTCTCCCCTCCCCTAGCCTGAAACCTGCTTGCTCGGGGTGGAGCTTCCTGCTCATTCGTTCTGCCAC | 332 | N/A |
| | | GCCCACTGCTGGAACCTGAGGAGCCACACACGTGCACCTTTCTACTGGACCAGAGATTATTCGGCGGGAA | | |
| | | TCGGGTCCCCTCCCCCTTCCTTCATAACTGGTGTCGCAACAATAAAATTT | | |
| ETnII-B3 R (full | N/A | GAGCCTTGATCA | 333 | N/A |
| length for v1 and | | | | |
| v3 design) | | | | |
| ETnII-B3 R (5′ | N/A | GCCTTGATCA | 334 | |
| truncated for v2 | | | | |
| design) | | | | |
| ETnII-B3 U5 | N/A | GAGTAACTGTCTTGGCTACATTTCTTCTTTTGCCCCGTCTAGATTCCTCTCTTACAGCTCGAGCGGCCTTCTC | 335 | N/A |
| | | AGTCGAACCGTTCACGTTGCGAGCTGCTGGCGGCCGCAACA | | |
| ETnII-B3 5′/3′ LTR | N/A | TGTAGTCTCCCCTCCCCTAGCCTGAAACCTGCTTGCTCGGGGTGGAGCTTCCTGCTCATTCGTTCTGCCAC | 336 | N/A |
| | | GCCCACTGCTGGAACCTGAGGAGCCACACACGTGCACCTTTCTACTGGACCAGAGATTATTCGGCGGGAA | | |
| | | TCGGGTCCCCTCCCCCTTCCTTCATAACTGGTGTCGCAACAATAAAATTTGAGCCTTGATCAGAGTAACTG | | |
| | | TCTTGGCTACATTTCTTCTTTTGCCCCGTCTAGATTCCTCTCTTACAGCTCGAGCGGCCTTCTCAGTCGAACC | | |
| | | GTTCACGTTGCGAGCTGCTGGCGGCCGCAACA | | |
| ETnII-B3 PBS | N/A | TGGCGCCCGAACAGGGAC | 337 | N/A |
| (shorter | | | | |
| annotation) | | | | |
| ETnII-B3 PBS | N/A | TTTTGGCGCCCGAACAGGGACCTGAAGAATGGC | 338 | N/A |
| (larger annotation) | | | | |
| ETnII-B3 5′ flank | N/A | AGAGAGATGCTAAGAGGAACGCTGCATTGGAGCTCCACAGGAAAGGATCTTCGTATCGGACATCGGAGC | 339 | N/A |
| | | AACGGACAAGTACACATGCTAGCGCTAGCTTAAAATTTCAGTTTTGTAAAGTGTTGCTGAGGATGCGGTA | | |
| | | GGATACGAATTAAGCTTGAATCAGTGCTAACCCAACGCTGGTTCTGCTTGGGTCAGCAGCGTGTTAATCG | | |
| | | GAACTAGAAACGGAAACAGGCAGGTTAGCCGCAGCTTTTTAGGAAGCTGCTTAGGTGAAAGAAGAAAG | | |
| | | GGTTTAAAGTCATGGATCAGGCGGTAGGCCGTAGCTCTCCGAAGCTACATGAGGTGTGAGAAAAGAAAG | | |
| | | GGTTTATTAAAAGGAATAGGCGGATTGCCCCAGTTAATAAAAAATACATCATAAGGGAGGAAAATGTCCC | | |
| | | AAAAAGCAGAGAGAAATATCTCTCTGGGCCTTATAGCAGGAGTACTCTGTTCCCCTTTGTGTCTTGTCTAA | | |
| | | TGTCCGGTGCACCAATCTGTTCTCGTGTTCGATTCATGTATGTTCGTGTCCAGTCTGTATGAATGAATGTTC | | |
| | | TATGTTTTGTGTTGGATAATAAAGATGGTATAAAAAACTTTATCTGCAAAGCCGAGAGCTGCCACGTGTTT | | |
| | | CAGCCAGGAATCAGACACGTGGCGAGAGGGCCCCTGCTGGAAAAACTGTTCGTTTTAGAAAATAAGGGC | | |
| | | GAGTGCACAGCCTCTAAGTTTCAGAGTAAAAAAGCTAATAAATGGTTCATGATTAATGTGTTTGACAATG | | |
| | | GTAAAGTGTTTTTTATTTTATGATTGTAGCTACAAAAATTATTATTCTCTGATTGGTCTAAATGTAACTGCTT | | |
| | | CATTTGGTTCTTTTTTATTGGTAATGTGCTCTAAGTGTTTTCACAATCAGCTCATAAGTTGTTGGTTAAGAT | | |
| | | TAATAATTGTTACATTGCTACAGATGGTTAGTGTTAAATTTGATAACTCAAGTTTAGAGTCCTTCCAACACA | | |
| | | TGGCATAAGGCAGCCCAAGAGGCTGGGTCTCTAAAGATATTTCTAGTTTAGGTAATAATTAATATGGTTC | | |
| | | GTATCCTAAATAGTAAAATTTAAAATAAGATTTAAAGCAATGTCTCTTTATTAAAGCATTAAAGCTTGCTTT | | |
| | | AATAGGTATTCATAGGTATTAATTGACATCCAAACTTCGTAATACGATAGTAATGCTTCTAATATTGATTTT | | |
| | | AAAGATAATAAATTGTTTTAAACTTGGGTTTTGCTTTCCCAAGGTTATAGGTATTATCCTAACCTTGCACAA | | |
| | | AAAACTTAAAAATTATGGTTAAAACTGCTATTGTTCCATTGACTGCAGCTTGCAGTTTGATTTCAAATTTAA | | |
| | | GATCTTTAATTCACCTGTATACTGTAATTAAGATAATTACAAGAGTAATCATCTTATGAGAGCGCTCATAC | | |
| | | AGCTCACTTCATACAAAACTGTGACAGAGTTATCTAGTTATGTTTGTCTTTGTGAATAGATTAGACAAACA | | |
| | | GTTTGGTTATAGTTGCTGCCTGGCAGGAAACTGTAGTACAGGCAATTTTTTCACAACACTAAAGTTGTGAA | | |
| | | GAACGTTTTAACTGTAAGTCATCTTAAGAAAGAGTATCAAAATTTAGAGGCGTAGACAGTTATATTGTTTC | | |
| | | TCTAAAATCGGTCCTTATTACAGGAGGGCCAGGATGCTCAGATTAAAAAGTATTTTACGTTTGAGTCAATG | | |
| | | CAGGGCTCTGGGCAGCCCCAGAGCGGCTTGTGGCCTTTCTTTTGTTTTGCAAACAGTGCCTGAGAAAGAT | | |
| | | TTTTCCCTGTGTTCAAGAAAAATTCTTTTTAACAGTGTTGCAGATCTATTCAGATGTTTAAAATAATGCTTA | | |
| | | AATTCAAAGAGTTTTGCTTCTAGTGAACTGTAATCACTAGAAAATTTTGCCTCTAGGTATGGCTAATGTAA | | |
| | | CTTTACATTATGTAAGAAAAATTTTATTGTTTCTGCTTCTATACAAGAAGCCAAGAGTTTTAATCTTTCAGT | | |
| | | GTATATTGTTTCCTAAGTGAAAAGTATTTTATTAACTAATGCTTCTTAAAGTTTACCTTAAATCCTTGCTCTC | | |
| | | ACCCAAAAGATTCAGAGACAATATCCTTTTATTACTTAGGGTTTTAGTTTACTACAAAAGTTTCTACAAAAA | | |
| | | ATAAAGCTTTTATAATTGTTATTAATTGGTAATTAAAAATTGGTTGTGCCCAAAACAATTCTTTGGCCAAAA | | |
| | | AAAAAAACATTATTGTAAAGTCATTTTTCTCATCCTCCCAGCCAATCGTTGGCCCACGTGGGCCCAACTAG | | |
| | | CTTCTGTGGGGCAGAGTCTTAAGACACAGTTTCCCCTGTTCCAGCACAGATGATCTAGTTGTGTGCTGTAG | | |
| | | ATGGTGTTTTAAAATGCTGAACAATCAAACCTTAATTTGTATATTAATAGTCAATGCCATATCTCTGAGCTC | | |
| | | ACAATTGCTTAAATTGTTCATCCCTCAGATACTATTAATTCTCAAATTTACAATTGCTTATGCATATTTCTAG | | |
| | | TTAATAAATAAATTATGCACATGTGACTCTTAATAACTTTACAAGCCTTCTAGTTACAACTGCTCCTTAAGA | | |
| | | AAATTGATTAAAAGTGCAATTAGTCACTGCCCCTTTACAGCCAAGTATTTAAAATGTTTTGTCAACTAGTTA | | |
| | | TTAATTCAAAAGTTTAGGTATTGTAAAATTTTAAAACTTTAACTTCTTAAAAGACAAAAAAGAGAGAAATT | | |
| | | GTATCCCCGTGCATGCTATTTAGT | | |
| ETnII-B3 3′ flank | N/A | GCATTCCCATGCACACTATTAAAGTCTTACCTTTATTTTCAAAATCTAATATTTTAATGTCAAAGAGTTTAAT | 340 | N/A |
| | | AAATGCTTTATAGTATCTTAAAGGGATCTACTTATTGGCTTATAGATTAATCCTAAAACAGCTACCTTATTA | | |
| | | AAAAAGGGAAAAACAGGTTTTCTCCACAGAACGCTGCAGAAGCATATTAATAAATTCGTGTGACGAGCTG | | |
| | | GTAGGTAAGTTGACTCATGTCCTGATTAAATTGACTAAAAAACTAAATTAAATTCATGTTTTAGATCCATCC | | |
| | | TTACTTGTCATTTTTCCAGTTTAGACTAGCTTCTAGCCTTTTAACTTTATGACAATAGTACATCAGAGACTGT | | |
| | | ATATTCAGACTTAGTAAAATTAGTCATTTAATAGAGTCATAATGATTTTTCTCCTTTCTTCAGTGTGACCAG | | |
| | | CCATTCTAACTCAATCTTAGACTGGTCCTAATATTCAGTCCAATGTTAGAGATTCCTATATTCTAAATTACCT | | |
| | | AGCAAGTTAATTAAAGAGCAATTGGTCACTTAATACCCCCTGACTAACAAATGGAAGGGTCCAGATCCAG | | |
| | | TTCTAATTTGGGGTAGGGACTCAGTTTGTGTTTTTTCACGAGATGAAGATGGAGCGCGGTGGCTGCCAGA | | |
| | | GAGATTAATTCGTCAGATGAACACAGATTCTGACTCTTCTGGTAAGTATCATTCTAAAGACTAAAATTCCT | | |
| | | TTTGTGCTTAAAATTCAGCTGAGAGCAACAGCTCTCAAAGGTGTTCTCCAGCTACTCTCTGAACCAGCTCC | | |
| | | CGACAGGAGGCCGGAGACTAGCCTCAGCTTTACAATTTGCATTTGAATAAAGTACCTAGACTTCCCGGAA | | |
| | | AGAAGTTCTGCTTTCCTACTTTCTCACTGTCTTTCAAGATTTTGTCTTTCAAGCAGGTAAATCAACATTCCCG | | |
| | | AGGCGGACCAGTAGATGTGCATCCCCGCCCCCCTAGAGCACACAGGTGGCAGCTGTTACCCCCAGTCTCA | | |
| | | GGACATTTCCAGCATGTGGCTTTCAGTCTGAGTTAAAAATTTAGGTTTACCTAGAGGGCTAGAAGAGTAG | | |
| | | ATTTTTCTATATTAATAAAGATTGGTTTTTATTTTGATAGACAGGCTTAGCCCCTTAGCTGACCTCTGGCTTT | | |
| | | TCACCCTTGCTGTTACTGCAAGGTGTCCTTAGCTCAATAGGCTGTGGAAAAAACAGGGATGAGGAGGAA | | |
| | | CGACTTCCAGCTCCTATTTTACCCACAAATCGTGGTGTTATTAACGACATAATTCTTGCTTAGGCTTTGCTA | | |
| | | ATTCTGAGGTTGATAATTCTCCTTTAGGAGCTGCACAGCACTCAGAACTGTGCATACTGGTTTGTGATTGT | | |
| | | ACAAATTCAGTATGGGCACCGCTTGGTGCAGAGATACTTACTGCAAGGGAAGGTCCGGCTTGACCATTTC | | |
| | | TGAGTTTCCTGTGAGATAAACCCGGTTTGAAAGAGGTTGGTACCAAATTTTGGTTAAAAATAAAAAATAT | | |
| | | TCTCCGGCTCTACCTCGCCTCCCCAAAAGGTACCAAGAGCCACATGTGTGGGTTTTACCCACGGGAGGAA | | |
| | | TCGGGTCCATGTCCACCCAAGCCAAGGTTAAAAGCCCACTCATCTACGGATGAGAAAATCATTTGATCACC | | |
| | | TCAGTTAAGCGTTGCCTTATTTAACTTAATTAATAGGGGGGAGAGAGATTGGAGACGTACTATTGAAAGG | | |
| | | GCAAGCCCTTCACTGCCTCCCACCCAAATAAAAAGGCCAATTGGCCTTGTACTACAAGAGCCGGTCACTCC | | |
| | | TTCTCCCTGTTTCCCACCTATCTTCCAAAAATGCTAAGGAATTCAACTTAGTGTTATTTTCACATCGTTCAGT | | |
| | | CAAACTTAGCCAGAGTTCCAAACGCCCTACTTAAAATTCAACTAGAAAGTTACCTACCAAGTACTAATTAG | | |
| | | CATTATAAAGTCAGAGTCTGCAGCTCCAGGCCTTTCAGTTGTTTACTAGAAAGGACAGTCTTAAGCCAGAT | | |
| | | ACAGTTTACCATAAGAAAAGTTAAAGATTCCCAGTGAAGCAAGTTTTTTCTTTAGCCCTAGATTCCAGGCA | | |
| | | GAACTATTGAGCATAGATAATTTTCCCCCC | | |
| ETnII-B3 PPT | N/A | AGGGAGGAGA | 341 | N/A |
| (shorter | | | | |
| annotation) | | | | |
| ETnII-B3 PPT | N/A | TCAGGCCAGCTTTTTCTTTTTTTTTTTATTTTGTTAATAACAGGGAGGAGA | 342 | N/A |
| (larger annotation) | | | | |
| TEMPLATE_COMPACT_ | PLV10059 | GAATTCGAGCTTGCATGCCTGCAGGTCGTTACATAACTTACGGTAAATGGCCCGCCTGGCTGACCGCCCA | 343 | N/A |
| pCMV_R-U5- | | ACGACCCCCGCCCATTGACGTCAATAATGACGTATGTTCCCATAGTAACGCCAATAGGGACTTTCCATTGA | | |
| ETnII-B3_EF1- | | CGTCAATGGGTGGAGTATTTACGGTAAACTGCCCACTTGGCAGTACATCAAGTGTATCATATGCCAAGTA | | |
| GFPai-LTR_v1 | | CGCCCCCTATTGACGTCAATGACGGTAAATGGCCCGCCTGGCATTATGCCCAGTACATGACCTTATGGGA | | |
| | | CTTTCCTACTTGGCAGTACATCTACGTATTAGTCATCGCTATTACCATGGTGATGCGGTTTTGGCAGTACAT | | |
| | | CAATGGGCGTGGATAGCGGTTTGACTCACGGGGATTTCCAAGTCTCCACCCCATTGACGTCAATGGGAGT | | |
| | | TTGTTTTGGCACCAAAATCAACGGGACTTTCCAAAATGTCGTAACAACTCCGCCCCATTGACGCAAATGGG | | |
| | | CGGTAGGCGTGTACGGTGGGAGGTCTATATAAGCAGAGCTCGTTTAGTGAACCGTCAGAGCCTTGATCA | | |
| | | GAGTAACTGTCTTGGCTACATTTCTTCTTTTGCCCCGTCTAGATTCCTCTCTTACAGCTCGAGCGGCCTTCTC | | |
| | | AGTCGAACCGTTCACGTTGCGAGCTGCTGGCGGCCGCAACATTTTGGCGCCCGAACAGGGACCTGAAGA | | |
| | | ATGGCgaacaaacgacccaacacccgtgcgttttattctgtctttttattgccgatcccccggccgctttacttgtacagctcgtccatgcc | | |
| | | gagagtgatcccggcggcggtcacgaactccagcaggaccatgtgatcgcgcttctcgttggggtctttgctcagggcggactgggtgctc | | |
| | | aggtaagtatcaaggttacaagacaggtttaaggagaccaatagaaactgggcttgtcgagacagagaagactcttgcgtttctgatagg | | |
| | | cacctattggtcttactgacatccactttgcctttctctccacaggtagtggttgtcgggcagcagcacggggccgtcgccgatgggggtgt | | |
| | | tctgctggtagtggtcggcgagctgcacgctgccgtcctcgatgttgtggcggatcttgaagttcaccttgatgccgttcttctgcttgtcg | | |
| | | gccatgatatagacgttgtggctgttgtagttgtactccagcttgtgccccaggatgttgccgtcctccttgaagtcgatgcccttcagctc | | |
| | | gatgcggttcaccagggtgtcgccctcgaacttcacctcggcgcgggtcttgtagttgccgtcgtccttgaagaagatggtgcgctcctgga | | |
| | | cgtagccttcgggcatggcggacttgaagaagtcgtgctgcttcatgtggtcggggtagcggctgaagcactgcacgccgtaggtcagggtg | | |
| | | gtcacgagggtgggccagggcacgggcagcttgccggtggtgcagatgaacttcagggtcagcttgccgtaggtggcategccctcgccctc | | |
| | | gccggacacgctgaacttgtggccgtttacgtcgccgtccagctcgaccaggatgggcaccaccccggtgaacagctcctcgcccttgctca | | |
| | | ccatggtggctttaccaacagtaccggaatgccaagcttgggtcctgtgttctggcggcaaacccgttgcgaaaaagaacgttcacggcgac | | |
| | | tactgcacttatatacggttctcccccaccctcgggaaaaaggcggagccagtacacgacatcactttcccagtttaccccgcgccaccttc | | |
| | | tctaggcaccggatcaattgccgacccctccccccaacttctcggggactgtgggcgatgtgcgctctgcccTCAGGCCAGCTTTTTCT | | |
| | | TTTTTTTTTTATTTTGTTAATAACAGGGAGGAGATGTAGTCTCCCCTCCCCTAGCCTGAAACCTGCTTGCTC | | |
| | | GGGGTGGAGCTTCCTGCTCATTCGTTCTGCCACGCCCACTGCTGGAACCTGAGGAGCCACACACGTGCAC | | |
| | | CTTTCTACTGGACCAGAGATTATTCGGCGGGAATCGGGTCCCCTCCCCCTTCCTTCATAACTGGTGTCGCA | | |
| | | ACAATAAAATTTGAGCCTTGATCAGAGTAACTGTCTTGGCTACATTTCTTCTTTTGCCCCGTCTAGATTCCT | | |
| | | CTCTTACAGCTCGAGCGGCCTTCTCAGTCGAACCGTTCACGTTGCGAGCTGCTGGCGGCCGCAACAGGGG | | |
| | | ATCCAGACATGATAAGATACATTGATGAGTTTGGACAAACCACAACTAGAATGCAGTGAAAAAAATGCTT | | |
| | | TATTTGTGAAATTTGTGATGCTATTGCTTTATTTGTAACCATTATAAGCTGCAATAAACAAGTTAACAACAA | | |
| | | CAATTGCATTCATTTTATGTTTCAGGTTCAGGGGGAGGTGTGGGAGGTTTTTTCGGATCCTCTAGAGTCGA | | |
| | | CCTGCAGGCATGCAAGCTTGGCGTAATCATGGTCATAGCTGTTTCCTGTGTGAAATTGTTATCCGCTCACA | | |
| | | ATTCCACACAACATACGAGCCGGAAGCATAAAGTGTAAAGCCTGGGGTGCCTAATGAGTGAGCTAACTC | | |
| | | ACATTAATTGCGTTGCGCTCACTGCCCGCTTTCCAGTCGGGAAACCTGTCGTGCCAGCTGCATTAATGAAT | | |
| | | CGGCCAACGCGCGGGGAGAGGCGGTTTGCGTATTGGGCGCTCTTCCGCTTCCTCGCTCACTGACTCGCTG | | |
| | | CGCTCGGTCGTTCGGCTGCGGCGAGCGGTATCAGCTCACTCAAAGGCGGTAATACGGTTATCCACAGAAT | | |
| | | CAGGGGATAACGCAGGAAAGAACATGTGAGCAAAAGGCCAGCAAAAGGCCAGGAACCGTAAAAAGGC | | |
| | | CGCGTTGCTGGCGTTTTTCCATAGGCTCCGCCCCCCTGACGAGCATCACAAAAATCGACGCTCAAGTCAG | | |
| | | AGGTGGCGAAACCCGACAGGACTATAAAGATACCAGGCGTTTCCCCCTGGAAGCTCCCTCGTGCGCTCTC | | |
| | | CTGTTCCGACCCTGCCGCTTACCGGATACCTGTCCGCCTTTCTCCCTTCGGGAAGCGTGGCGCTTTCTCATA | | |
| | | GCTCACGCTGTAGGTATCTCAGTTCGGTGTAGGTCGTTCGCTCCAAGCTGGGCTGTGTGCACGAACCCCC | | |
| | | CGTTCAGCCCGACCGCTGCGCCTTATCCGGTAACTATCGTCTTGAGTCCAACCCGGTAAGACACGACTTAT | | |
| | | CGCCACTGGCAGCAGCCACTGGTAACAGGATTAGCAGAGCGAGGTATGTAGGCGGTGCTACAGAGTTCT | | |
| | | TGAAGTGGTGGCCTAACTACGGCTACACTAGAAGGACAGTATTTGGTATCTGCGCTCTGCTGAAGCCAGT | | |
| | | TACCTTCGGAAAAAGAGTTGGTAGCTCTTGATCCGGCAAACAAACCACCGCTGGTAGCGGTGGTTTTTTT | | |
| | | GTTTGCAAGCAGCAGATTACGCGCAGAAAAAAAGGATCTCAAGAAGATCCTTTGATCTTTTCTACGGGGT | | |
| | | CTGACGCTCAGTGGAACGAAAACTCACGTTAAGGGATTTTGGTCATGAGATTATCAAAAAGGATCTTCAC | | |
| | | CTAGATCCTTTTAAATTAAAAATGAAGTTTTAAATCAATCTAAAGTATATATGAGTAAACTTGGTCTGACA | | |
| | | GTTACCAATGCTTAATCAGTGAGGCACCTATCTCAGCGATCTGTCTATTTCGTTCATCCATAGTTGCCTGAC | | |
| | | TCCCCGTCGTGTAGATAACTACGATACGGGAGGGCTTACCATCTGGCCCCAGTGCTGCAATGATACCGCG | | |
| | | AGACCCACGCTCACCGGCTCCAGATTTATCAGCAATAAACCAGCCAGCCGGAAGGGCCGAGCGCAGAAG | | |
| | | TGGTCCTGCAACTTTATCCGCCTCCATCCAGTCTATTAATTGTTGCCGGGAAGCTAGAGTAAGTAGTTCGC | | |
| | | CAGTTAATAGTTTGCGCAACGTTGTTGCCATTGCTACAGGCATCGTGGTGTCACGCTCGTCGTTTGGTATG | | |
| | | GCTTCATTCAGCTCCGGTTCCCAACGATCAAGGCGAGTTACATGATCCCCCATGTTGTGCAAAAAAGCGG | | |
| | | TTAGCTCCTTCGGTCCTCCGATCGTTGTCAGAAGTAAGTTGGCCGCAGTGTTATCACTCATGGTTATGGCA | | |
| | | GCACTGCATAATTCTCTTACTGTCATGCCATCCGTAAGATGCTTTTCTGTGACTGGTGAGTACTCAACCAA | | |
| | | GTCATTCTGAGAATAGTGTATGCGGCGACCGAGTTGCTCTTGCCCGGCGTCAATACGGGATAATACCGCG | | |
| | | CCACATAGCAGAACTTTAAAAGTGCTCATCATTGGAAAACGTTCTTCGGGGCGAAAACTCTCAAGGATCT | | |
| | | TACCGCTGTTGAGATCCAGTTCGATGTAACCCACTCGTGCACCCAACTGATCTTCAGCATCTTTTACTTTCA | | |
| | | CCAGCGTTTCTGGGTGAGCAAAAACAGGAAGGCAAAATGCCGCAAAAAAGGGAATAAGGGCGACACGG | | |
| | | AAATGTTGAATACTCATACTCTTCCTTTTTCAATATTATTGAAGCATTTATCAGGGTTATTGTCTCATGAGC | | |
| | | GGATACATATTTGAATGTATTTAGAAAAATAAACAAATAGGGGTTCCGCGCACATTTCCCCGAAAAGTGC | | |
| | | CACCTGACGTCTAAGAAACCATTATTATCATGACATTAACCTATAAAAATAGGCGTATCACGAGGCCCTTT | | |
| | | CGTCTCGCGCGTTTCGGTGATGACGGTGAAAACCTCTGACACATGCAGCTCCCGGAGACGGTCACAGCTT | | |
| | | GTCTGTAAGCGGATGCCGGGAGCAGACAAGCCCGTCAGGGCGCGTCAGCGGGTGTTGGCGGGTGTCGG | | |
| | | GGCTGGCTTAACTATGCGGCATCAGAGCAGATTGTACTGAGAGTGCACCATATGCGGTGTGAAATACCG | | |
| | | CACAGATGCGTAAGGAGAAAATACCGCATCAGGCGCCATTCGCCATTCAGGCTGCGCAACTGTTGGGAA | | |
| | | GGGCGATCGGTGCGGGCCTCTTCGCTATTACGCCAGCTGGCGAAAGGGGGATGTGCTGCAAGGCGATTA | | |
| | | AGTTGGGTAACGCCAGGGTTTTCCCAGTCACGACGTTGTAAAACGACGGCCAGT | | |
| TEMPLATE_COMPACT_ | PLV10060 | GAATTCGAGCTTGCATGCCTGCAGGTCGTTACATAACTTACGGTAAATGGCCCGCCTGGCTGACCGCCCA | 344 | N/A |
| pCMV_R-U5- | | ACGACCCCCGCCCATTGACGTCAATAATGACGTATGTTCCCATAGTAACGCCAATAGGGACTTTCCATTGA | | |
| ETnII-B3_EF1- | | CGTCAATGGGTGGAGTATTTACGGTAAACTGCCCACTTGGCAGTACATCAAGTGTATCATATGCCAAGTA | | |
| GFPai-LTR_v2 | | CGCCCCCTATTGACGTCAATGACGGTAAATGGCCCGCCTGGCATTATGCCCAGTACATGACCTTATGGGA | | |
| | | CTTTCCTACTTGGCAGTACATCTACGTATTAGTCATCGCTATTACCATGGTGATGCGGTTTTGGCAGTACAT | | |
| | | CAATGGGCGTGGATAGCGGTTTGACTCACGGGGATTTCCAAGTCTCCACCCCATTGACGTCAATGGGAGT | | |
| | | TTGTTTTGGCACCAAAATCAACGGGACTTTCCAAAATGTCGTAACAACTCCGCCCCATTGACGCAAATGGG | | |
| | | CGGTAGGCGTGTACGGTGGGAGGTCTATATAAGCAGAGCTCGTTTAGTGAACCGTCAGCCTTGATCAGA | | |
| | | GTAACTGTCTTGGCTACATTTCTTCTTTTGCCCCGTCTAGATTCCTCTCTTACAGCTCGAGCGGCCTTCTCAG | | |
| | | TCGAACCGTTCACGTTGCGAGCTGCTGGCGGCCGCAACATTTTGGCGCCCGAACAGGGACCTGAAGAAT | | |
| | | GGCgaacaaacgacccaacacccgtgcgttttattctgtctttttattgccgatcccccggccgctttacttgtacagctcgtccatgccgag | | |
| | | agtgatcccggcggcggtcacgaactccagcaggaccatgtgatcgcgcttctcgttggggtctttgctcagggcggactgggtgctcaggt | | |
| | | aagtatcaaggttacaagacaggtttaaggagaccaatagaaactgggcttgtcgagacagagaagactcttgcgtttctgataggcacc | | |
| | | tattggtcttactgacatccactttgcctttctctccacaggtagtggttgtcgggcagcagcacggggccgtcgccgatgggggtgttctgct | | |
| | | ggtagtggtcggcgagctgcacgctgccgtcctcgatgttgtggcggatcttgaagttcaccttgatgccgttcttctgcttgtcggccatgat | | |
| | | atagacgttgtggctgttgtagttgtactccagcttgtgccccaggatgttgccgtcctccttgaagtcgatgcccttcagctcgatgcggttc | | |
| | | accagggtgtcgccctcgaacttcacctcggcgcgggtcttgtagttgccgtcgtccttgaagaagatggtgcgctcctggacgtagccttcg | | |
| | | ggcatggcggacttgaagaagtcgtgctgcttcatgtggtcggggtagcggctgaagcactgcacgccgtaggtcagggtggtcacgagg | | |
| | | gtgggccagggcacgggcagcttgccggtggtgcagatgaacttcagggtcagcttgccgtaggtggcatcgccctcgccctcgccggaca | | |
| | | cgctgaacttgtggccgtttacgtcgccgtccagctcgaccaggatgggcaccaccccggtgaacagctcctcgcccttgctcaccatggtg | | |
| | | gctttaccaacagtaccggaatgccaagcttgggtcctgtgttctggcggcaaacccgttgcgaaaaagaacgttcacggcgactactgca | | |
| | | cttatatacggttctcccccaccctcgggaaaaaggcggagccagtacacgacatcactttcccagtttaccccgcgccaccttctctaggc | | |
| | | accggatcaattgccgacccctccccccaacttctcggggactgtgggcgatgtgcgctctgcccTCAGGCCAGCTTTTTCTTTTTT | | |
| | | TTTTTATTTTGTTAATAACAGGGAGGAGATGTAGTCTCCCCTCCCCTAGCCTGAAACCTGCTTGCTCGGGG | | |
| | | TGGAGCTTCCTGCTCATTCGTTCTGCCACGCCCACTGCTGGAACCTGAGGAGCCACACACGTGCACCTTTC | | |
| | | TACTGGACCAGAGATTATTCGGCGGGAATCGGGTCCCCTCCCCCTTCCTTCATAACTGGTGTCGCAACAAT | | |
| | | AAAATTTGAGCCTTGATCAGAGTAACTGTCTTGGCTACATTTCTTCTTTTGCCCCGTCTAGATTCCTCTCTTA | | |
| | | CAGCTCGAGCGGCCTTCTCAGTCGAACCGTTCACGTTGCGAGCTGCTGGCGGCCGCAACAGGGGATCCA | | |
| | | GACATGATAAGATACATTGATGAGTTTGGACAAACCACAACTAGAATGCAGTGAAAAAAATGCTTTATTT | | |
| | | GTGAAATTTGTGATGCTATTGCTTTATTTGTAACCATTATAAGCTGCAATAAACAAGTTAACAACAACAAT | | |
| | | TGCATTCATTTTATGTTTCAGGTTCAGGGGGAGGTGTGGGAGGTTTTTTCGGATCCTCTAGAGTCGACCTG | | |
| | | CAGGCATGCAAGCTTGGCGTAATCATGGTCATAGCTGTTTCCTGTGTGAAATTGTTATCCGCTCACAATTC | | |
| | | CACACAACATACGAGCCGGAAGCATAAAGTGTAAAGCCTGGGGTGCCTAATGAGTGAGCTAACTCACAT | | |
| | | TAATTGCGTTGCGCTCACTGCCCGCTTTCCAGTCGGGAAACCTGTCGTGCCAGCTGCATTAATGAATCGGC | | |
| | | CAACGCGCGGGGAGAGGCGGTTTGCGTATTGGGCGCTCTTCCGCTTCCTCGCTCACTGACTCGCTGCGCT | | |
| | | CGGTCGTTCGGCTGCGGCGAGCGGTATCAGCTCACTCAAAGGCGGTAATACGGTTATCCACAGAATCAG | | |
| | | GGGATAACGCAGGAAAGAACATGTGAGCAAAAGGCCAGCAAAAGGCCAGGAACCGTAAAAAGGCCGC | | |
| | | GTTGCTGGCGTTTTTCCATAGGCTCCGCCCCCCTGACGAGCATCACAAAAATCGACGCTCAAGTCAGAGG | | |
| | | TGGCGAAACCCGACAGGACTATAAAGATACCAGGCGTTTCCCCCTGGAAGCTCCCTCGTGCGCTCTCCTG | | |
| | | TTCCGACCCTGCCGCTTACCGGATACCTGTCCGCCTTTCTCCCTTCGGGAAGCGTGGCGCTTTCTCATAGCT | | |
| | | CACGCTGTAGGTATCTCAGTTCGGTGTAGGTCGTTCGCTCCAAGCTGGGCTGTGTGCACGAACCCCCCGT | | |
| | | TCAGCCCGACCGCTGCGCCTTATCCGGTAACTATCGTCTTGAGTCCAACCCGGTAAGACACGACTTATCGC | | |
| | | CACTGGCAGCAGCCACTGGTAACAGGATTAGCAGAGCGAGGTATGTAGGCGGTGCTACAGAGTTCTTGA | | |
| | | AGTGGTGGCCTAACTACGGCTACACTAGAAGGACAGTATTTGGTATCTGCGCTCTGCTGAAGCCAGTTAC | | |
| | | CTTCGGAAAAAGAGTTGGTAGCTCTTGATCCGGCAAACAAACCACCGCTGGTAGCGGTGGTTTTTTTGTTT | | |
| | | GCAAGCAGCAGATTACGCGCAGAAAAAAAGGATCTCAAGAAGATCCTTTGATCTTTTCTACGGGGTCTGA | | |
| | | CGCTCAGTGGAACGAAAACTCACGTTAAGGGATTTTGGTCATGAGATTATCAAAAAGGATCTTCACCTAG | | |
| | | ATCCTTTTAAATTAAAAATGAAGTTTTAAATCAATCTAAAGTATATATGAGTAAACTTGGTCTGACAGTTAC | | |
| | | CAATGCTTAATCAGTGAGGCACCTATCTCAGCGATCTGTCTATTTCGTTCATCCATAGTTGCCTGACTCCCC | | |
| | | GTCGTGTAGATAACTACGATACGGGAGGGCTTACCATCTGGCCCCAGTGCTGCAATGATACCGCGAGACC | | |
| | | CACGCTCACCGGCTCCAGATTTATCAGCAATAAACCAGCCAGCCGGAAGGGCCGAGCGCAGAAGTGGTC | | |
| | | CTGCAACTTTATCCGCCTCCATCCAGTCTATTAATTGTTGCCGGGAAGCTAGAGTAAGTAGTTCGCCAGTT | | |
| | | AATAGTTTGCGCAACGTTGTTGCCATTGCTACAGGCATCGTGGTGTCACGCTCGTCGTTTGGTATGGCTTC | | |
| | | ATTCAGCTCCGGTTCCCAACGATCAAGGCGAGTTACATGATCCCCCATGTTGTGCAAAAAAGCGGTTAGC | | |
| | | TCCTTCGGTCCTCCGATCGTTGTCAGAAGTAAGTTGGCCGCAGTGTTATCACTCATGGTTATGGCAGCACT | | |
| | | GCATAATTCTCTTACTGTCATGCCATCCGTAAGATGCTTTTCTGTGACTGGTGAGTACTCAACCAAGTCATT | | |
| | | CTGAGAATAGTGTATGCGGCGACCGAGTTGCTCTTGCCCGGCGTCAATACGGGATAATACCGCGCCACAT | | |
| | | AGCAGAACTTTAAAAGTGCTCATCATTGGAAAACGTTCTTCGGGGCGAAAACTCTCAAGGATCTTACCGC | | |
| | | TGTTGAGATCCAGTTCGATGTAACCCACTCGTGCACCCAACTGATCTTCAGCATCTTTTACTTTCACCAGCG | | |
| | | TTTCTGGGTGAGCAAAAACAGGAAGGCAAAATGCCGCAAAAAAGGGAATAAGGGCGACACGGAAATGT | | |
| | | TGAATACTCATACTCTTCCTTTTTCAATATTATTGAAGCATTTATCAGGGTTATTGTCTCATGAGCGGATAC | | |
| | | ATATTTGAATGTATTTAGAAAAATAAACAAATAGGGGTTCCGCGCACATTTCCCCGAAAAGTGCCACCTG | | |
| | | ACGTCTAAGAAACCATTATTATCATGACATTAACCTATAAAAATAGGCGTATCACGAGGCCCTTTCGTCTC | | |
| | | GCGCGTTTCGGTGATGACGGTGAAAACCTCTGACACATGCAGCTCCCGGAGACGGTCACAGCTTGTCTGT | | |
| | | AAGCGGATGCCGGGAGCAGACAAGCCCGTCAGGGCGCGTCAGCGGGTGTTGGCGGGTGTCGGGGCTG | | |
| | | GCTTAACTATGCGGCATCAGAGCAGATTGTACTGAGAGTGCACCATATGCGGTGTGAAATACCGCACAG | | |
| | | ATGCGTAAGGAGAAAATACCGCATCAGGCGCCATTCGCCATTCAGGCTGCGCAACTGTTGGGAAGGGCG | | |
| | | ATCGGTGCGGGCCTCTTCGCTATTACGCCAGCTGGCGAAAGGGGGATGTGCTGCAAGGCGATTAAGTTG | | |
| | | GGTAACGCCAGGGTTTTCCCAGTCACGACGTTGTAAAACGACGGCCAGT | | |
| TEMPLATE_COMPACT_ | PLV10061 | GAATTCGAGCTTGCATGCCTGCAGGTCGTTACATAACTTACGGTAAATGGCCCGCCTGGCTGACCGCCCA | 345 | N/A |
| pCMV_R-U5- | | ACGACCCCCGCCCATTGACGTCAATAATGACGTATGTTCCCATAGTAACGCCAATAGGGACTTTCCATTGA | | |
| ETnII-B3_EF1- | | CGTCAATGGGTGGAGTATTTACGGTAAACTGCCCACTTGGCAGTACATCAAGTGTATCATATGCCAAGTA | | |
| GFPai-LTR_v3 | | CGCCCCCTATTGACGTCAATGACGGTAAATGGCCCGCCTGGCATTATGCCCAGTACATGACCTTATGGGA | | |
| | | CTTTCCTACTTGGCAGTACATCTACGTATTAGTCATCGCTATTACCATGGTGATGCGGTTTTGGCAGTACAT | | |
| | | CAATGGGCGTGGATAGCGGTTTGACTCACGGGGATTTCCAAGTCTCCACCCCATTGACGTCAATGGGAGT | | |
| | | TTGTTTTGGCACCAAAATCAACGGGACTTTCCAAAATGTCGTAACAACTCCGCCCCATTGACGCAAATGGG | | |
| | | CGGTAGGCGTGTACGGTGGGAGGTCTATATAAGCAGAGCTGAGCCTTGATCAGAGTAACTGTCTTGGCT | | |
| | | ACATTTCTTCTTTTGCCCCGTCTAGATTCCTCTCTTACAGCTCGAGCGGCCTTCTCAGTCGAACCGTTCACG | | |
| | | TTGCGAGCTGCTGGCGGCCGCAACATTTTGGCGCCCGAACAGGGACCTGAAGAATGGCgaacaaacgaccca | | |
| | | acacccgtgcgttttattctgtctttttattgccgatcccccggccgctttacttgtacagctcgtccatgccgagagtgatcccggcggcg | | |
| | | gtcacgaactccagcaggaccatgtgatcgcgcttctcgttggggtctttgctcagggcggactgggtgctcaggtaagtatcaaggttaca | | |
| | | agacaggtttaaggagaccaatagaaactgggcttgtcgagacagagaagactcttgcgtttctgataggcacctattggtcttactgacat | | |
| | | ccactttgcctttctctccacaggtagtggttgtcgggcagcagcacggggccgtcgccgatgggggtgttctgctggtagtggtcggcgag | | |
| | | ctgcacgctgccgtcctcgatgttgtggcggatcttgaagttcaccttgatgccgttcttctgcttgtcggccatgatatagacgttgtggc | | |
| | | tgttgtagttgtactccagcttgtgccccaggatgttgccgtcctccttgaagtcgatgcccttcagctcgatgcggttcaccagggtgtcg | | |
| | | ccctcgaacttcacctcggcgcgggtcttgtagttgccgtcgtccttgaagaagatggtgcgctcctggacgtagccttcgggcatggcgga | | |
| | | cttgaagaagtcgtgctgcttcatgtggtcggggtagcggctgaagcactgcacgccgtaggtcagggtggtcacgagggtgggccagggca | | |
| | | cgggcagcttgccggtggtgcagatgaacttcagggtcagcttgccgtaggtggcatcgccctcgccctcgccggacacgctgaacttgtgg | | |
| | | ccgtttacgtcgccgtccagctcgaccaggatgggcaccaccccggtgaacagctcctcgcccttgctcaccatggtggctttaccaacagt | | |
| | | accggaatgccaagcttgggtcctgtgttctggcggcaaacccgttgcgaaaaagaacgttcacggcgactactgcacttatatacggttct | | |
| | | cccccaccctcgggaaaaaggcggagccagtacacgacatcactttcccagtttaccccgcgccaccttctctaggcaccggatcaattgcc | | |
| | | gacccctccccccaacttctcggggactgtgggcgatgtgcgctctgcccTCAGGCCAGCTTTTTCTTTTTTTTTTTATTTTGTTAA | | |
| | | TAACAGGGAGGAGATGTAGTCTCCCCTCCCCTAGCCTGAAACCTGCTTGCTCGGGGTGGAGCTTCCTGCT | | |
| | | CATTCGTTCTGCCACGCCCACTGCTGGAACCTGAGGAGCCACACACGTGCACCTTTCTACTGGACCAGAG | | |
| | | ATTATTCGGCGGGAATCGGGTCCCCTCCCCCTTCCTTCATAACTGGTGTCGCAACAATAAAATTTGAGCCT | | |
| | | TGATCAGAGTAACTGTCTTGGCTACATTTCTTCTTTTGCCCCGTCTAGATTCCTCTCTTACAGCTCGAGCGG | | |
| | | CCTTCTCAGTCGAACCGTTCACGTTGCGAGCTGCTGGCGGCCGCAACAGGGGATCCAGACATGATAAGAT | | |
| | | ACATTGATGAGTTTGGACAAACCACAACTAGAATGCAGTGAAAAAAATGCTTTATTTGTGAAATTTGTGA | | |
| | | TGCTATTGCTTTATTTGTAACCATTATAAGCTGCAATAAACAAGTTAACAACAACAATTGCATTCATTTTAT | | |
| | | GTTTCAGGTTCAGGGGGAGGTGTGGGAGGTTTTTTCGGATCCTCTAGAGTCGACCTGCAGGCATGCAAG | | |
| | | CTTGGCGTAATCATGGTCATAGCTGTTTCCTGTGTGAAATTGTTATCCGCTCACAATTCCACACAACATACG | | |
| | | AGCCGGAAGCATAAAGTGTAAAGCCTGGGGTGCCTAATGAGTGAGCTAACTCACATTAATTGCGTTGCG | | |
| | | CTCACTGCCCGCTTTCCAGTCGGGAAACCTGTCGTGCCAGCTGCATTAATGAATCGGCCAACGCGCGGGG | | |
| | | AGAGGCGGTTTGCGTATTGGGCGCTCTTCCGCTTCCTCGCTCACTGACTCGCTGCGCTCGGTCGTTCGGCT | | |
| | | GCGGCGAGCGGTATCAGCTCACTCAAAGGCGGTAATACGGTTATCCACAGAATCAGGGGATAACGCAGG | | |
| | | AAAGAACATGTGAGCAAAAGGCCAGCAAAAGGCCAGGAACCGTAAAAAGGCCGCGTTGCTGGCGTTTTT | | |
| | | CCATAGGCTCCGCCCCCCTGACGAGCATCACAAAAATCGACGCTCAAGTCAGAGGTGGCGAAACCCGAC | | |
| | | AGGACTATAAAGATACCAGGCGTTTCCCCCTGGAAGCTCCCTCGTGCGCTCTCCTGTTCCGACCCTGCCGC | | |
| | | TTACCGGATACCTGTCCGCCTTTCTCCCTTCGGGAAGCGTGGCGCTTTCTCATAGCTCACGCTGTAGGTAT | | |
| | | CTCAGTTCGGTGTAGGTCGTTCGCTCCAAGCTGGGCTGTGTGCACGAACCCCCCGTTCAGCCCGACCGCT | | |
| | | GCGCCTTATCCGGTAACTATCGTCTTGAGTCCAACCCGGTAAGACACGACTTATCGCCACTGGCAGCAGC | | |
| | | CACTGGTAACAGGATTAGCAGAGCGAGGTATGTAGGCGGTGCTACAGAGTTCTTGAAGTGGTGGCCTAA | | |
| | | CTACGGCTACACTAGAAGGACAGTATTTGGTATCTGCGCTCTGCTGAAGCCAGTTACCTTCGGAAAAAGA | | |
| | | GTTGGTAGCTCTTGATCCGGCAAACAAACCACCGCTGGTAGCGGTGGTTTTTTTGTTTGCAAGCAGCAGA | | |
| | | TTACGCGCAGAAAAAAAGGATCTCAAGAAGATCCTTTGATCTTTTCTACGGGGTCTGACGCTCAGTGGAA | | |
| | | CGAAAACTCACGTTAAGGGATTTTGGTCATGAGATTATCAAAAAGGATCTTCACCTAGATCCTTTTAAATT | | |
| | | AAAAATGAAGTTTTAAATCAATCTAAAGTATATATGAGTAAACTTGGTCTGACAGTTACCAATGCTTAATC | | |
| | | AGTGAGGCACCTATCTCAGCGATCTGTCTATTTCGTTCATCCATAGTTGCCTGACTCCCCGTCGTGTAGATA | | |
| | | ACTACGATACGGGAGGGCTTACCATCTGGCCCCAGTGCTGCAATGATACCGCGAGACCCACGCTCACCGG | | |
| | | CTCCAGATTTATCAGCAATAAACCAGCCAGCCGGAAGGGCCGAGCGCAGAAGTGGTCCTGCAACTTTATC | | |
| | | CGCCTCCATCCAGTCTATTAATTGTTGCCGGGAAGCTAGAGTAAGTAGTTCGCCAGTTAATAGTTTGCGCA | | |
| | | ACGTTGTTGCCATTGCTACAGGCATCGTGGTGTCACGCTCGTCGTTTGGTATGGCTTCATTCAGCTCCGGT | | |
| | | TCCCAACGATCAAGGCGAGTTACATGATCCCCCATGTTGTGCAAAAAAGCGGTTAGCTCCTTCGGTCCTCC | | |
| | | GATCGTTGTCAGAAGTAAGTTGGCCGCAGTGTTATCACTCATGGTTATGGCAGCACTGCATAATTCTCTTA | | |
| | | CTGTCATGCCATCCGTAAGATGCTTTTCTGTGACTGGTGAGTACTCAACCAAGTCATTCTGAGAATAGTGT | | |
| | | ATGCGGCGACCGAGTTGCTCTTGCCCGGCGTCAATACGGGATAATACCGCGCCACATAGCAGAACTTTAA | | |
| | | AAGTGCTCATCATTGGAAAACGTTCTTCGGGGCGAAAACTCTCAAGGATCTTACCGCTGTTGAGATCCAG | | |
| | | TTCGATGTAACCCACTCGTGCACCCAACTGATCTTCAGCATCTTTTACTTTCACCAGCGTTTCTGGGTGAGC | | |
| | | AAAAACAGGAAGGCAAAATGCCGCAAAAAAGGGAATAAGGGCGACACGGAAATGTTGAATACTCATAC | | |
| | | TCTTCCTTTTTCAATATTATTGAAGCATTTATCAGGGTTATTGTCTCATGAGCGGATACATATTTGAATGTA | | |
| | | TTTAGAAAAATAAACAAATAGGGGTTCCGCGCACATTTCCCCGAAAAGTGCCACCTGACGTCTAAGAAAC | | |
| | | CATTATTATCATGACATTAACCTATAAAAATAGGCGTATCACGAGGCCCTTTCGTCTCGCGCGTTTCGGTG | | |
| | | ATGACGGTGAAAACCTCTGACACATGCAGCTCCCGGAGACGGTCACAGCTTGTCTGTAAGCGGATGCCG | | |
| | | GGAGCAGACAAGCCCGTCAGGGCGCGTCAGCGGGTGTTGGCGGGTGTCGGGGCTGGCTTAACTATGCG | | |
| | | GCATCAGAGCAGATTGTACTGAGAGTGCACCATATGCGGTGTGAAATACCGCACAGATGCGTAAGGAGA | | |
| | | AAATACCGCATCAGGCGCCATTCGCCATTCAGGCTGCGCAACTGTTGGGAAGGGCGATCGGTGCGGGCC | | |
| | | TCTTCGCTATTACGCCAGCTGGCGAAAGGGGGATGTGCTGCAAGGCGATTAAGTTGGGTAACGCCAGGG | | |
| | | TTTTCCCAGTCACGACGTTGTAAAACGACGGCCAGT | | |
| TEMPLATE_pCMV | PLV10069 | GAATTCGAGCTTGCATGCCTGCAGGTCGTTACATAACTTACGGTAAATGGCCCGCCTGGCTGACCGCCCA | 346 | N/A |
| R-U5-ETnII- | | ACGACCCCCGCCCATTGACGTCAATAATGACGTATGTTCCCATAGTAACGCCAATAGGGACTTTCCATTGA | | |
| B3_EF1-GFPai- | | CGTCAATGGGTGGAGTATTTACGGTAAACTGCCCACTTGGCAGTACATCAAGTGTATCATATGCCAAGTA | | |
| LTR_v1 | | CGCCCCCTATTGACGTCAATGACGGTAAATGGCCCGCCTGGCATTATGCCCAGTACATGACCTTATGGGA | | |
| | | CTTTCCTACTTGGCAGTACATCTACGTATTAGTCATCGCTATTACCATGGTGATGCGGTTTTGGCAGTACAT | | |
| | | CAATGGGCGTGGATAGCGGTTTGACTCACGGGGATTTCCAAGTCTCCACCCCATTGACGTCAATGGGAGT | | |
| | | TTGTTTTGGCACCAAAATCAACGGGACTTTCCAAAATGTCGTAACAACTCCGCCCCATTGACGCAAATGGG | | |
| | | CGGTAGGCGTGTACGGTGGGAGGTCTATATAAGCAGAGCTCGTTTAGTGAACCGTCAGCCTTGATCAGA | | |
| | | GTAACTGTCTTGGCTACATTTCTTCTTTTGCCCCGTCTAGATTCCTCTCTTACAGCTCGAGCGGCCTTCTCAG | | |
| | | TCGAACCGTTCACGTTGCGAGCTGCTGGCGGCCGCAACATTTTGGCGCCCGAACAGGGACCTGAAGAAT | | |
| | | GGCAGAGAGATGCTAAGAGGAACGCTGCATTGGAGCTCCACAGGAAAGGATCTTCGTATCGGACATCGG | | |
| | | AGCAACGGACAAGTACACATGCTAGCGCTAGCTTAAAATTTCAGTTTTGTAAAGTGTTGCTGAGGATGCG | | |
| | | GTAGGATACGAATTAAGCTTGAATCAGTGCTAACCCAACGCTGGTTCTGCTTGGGTCAGCAGCGTGTTAA | | |
| | | TCGGAACTAGAAACGGAAACAGGCAGGTTAGCCGCAGCTTTTTAGGAAGCTGCTTAGGTGAAAGAAGAA | | |
| | | AGGGTTTAAAGTCATGGATCAGGCGGTAGGCCGTAGCTCTCCGAAGCTACATGAGGTGTGAGAAAAGAA | | |
| | | AGGGTTTATTAAAAGGAATAGGCGGATTGCCCCAGTTAATAAAAAATACATCATAAGGGAGGAAAATGT | | |
| | | CCCAAAAAGCAGAGAGAAATATCTCTCTGGGCCTTATAGCAGGAGTACTCTGTTCCCCTTTGTGTCTTGTC | | |
| | | TAATGTCCGGTGCACCAATCTGTTCTCGTGTTCGATTCATGTATGTTCGTGTCCAGTCTGTATGAATGAAT | | |
| | | GTTCTATGTTTTGTGTTGGATAATAAAGATGGTATAAAAAACTTTATCTGCAAAGCCGAGAGCTGCCACGT | | |
| | | GTTTCAGCCAGGAATCAGACACGTGGCGAGAGGGCCCCTGCTGGAAAAACTGTTCGTTTTAGAAAATAA | | |
| | | GGGCGAGTGCACAGCCTCTAAGTTTCAGAGTAAAAAAGCTAATAAATGGTTCATGATTAATGTGTTTGAC | | |
| | | AATGGTAAAGTGTTTTTTATTTTATGATTGTAGCTACAAAAATTATTATTCTCTGATTGGTCTAAATGTAAC | | |
| | | TGCTTCATTTGGTTCTTTTTTATTGGTAATGTGCTCTAAGTGTTTTCACAATCAGCTCATAAGTTGTTGGTTA | | |
| | | AGATTAATAATTGTTACATTGCTACAGATGGTTAGTGTTAAATTTGATAACTCAAGTTTAGAGTCCTTCCAA | | |
| | | CACATGGCATAAGGCAGCCCAAGAGGCTGGGTCTCTAAAGATATTTCTAGTTTAGGTAATAATTAATATG | | |
| | | GTTCGTATCCTAAATAGTAAAATTTAAAATAAGATTTAAAGCAATGTCTCTTTATTAAAGCATTAAAGCTTG | | |
| | | CTTTAATAGGTATTCATAGGTATTAATTGACATCCAAACTTCGTAATACGATAGTAATGCTTCTAATATTGA | | |
| | | TTTTAAAGATAATAAATTGTTTTAAACTTGGGTTTTGCTTTCCCAAGGTTATAGGTATTATCCTAACCTTGC | | |
| | | ACAAAAAACTTAAAAATTATGGTTAAAACTGCTATTGTTCCATTGACTGCAGCTTGCAGTTTGATTTCAAAT | | |
| | | TTAAGATCTTTAATTCACCTGTATACTGTAATTAAGATAATTACAAGAGTAATCATCTTATGAGAGCGCTCA | | |
| | | TACAGCTCACTTCATACAAAACTGTGACAGAGTTATCTAGTTATGTTTGTCTTTGTGAATAGATTAGACAA | | |
| | | ACAGTTTGGTTATAGTTGCTGCCTGGCAGGAAACTGTAGTACAGGCAATTTTTTCACAACACTAAAGTTGT | | |
| | | GAAGAACGTTTTAACTGTAAGTCATCTTAAGAAAGAGTATCAAAATTTAGAGGCGTAGACAGTTATATTG | | |
| | | TTTCTCTAAAATCGGTCCTTATTACAGGAGGGCCAGGATGCTCAGATTAAAAAGTATTTTACGTTTGAGTC | | |
| | | AATGCAGGGCTCTGGGCAGCCCCAGAGCGGCTTGTGGCCTTTCTTTTGTTTTGCAAACAGTGCCTGAGAA | | |
| | | AGATTTTTCCCTGTGTTCAAGAAAAATTCTTTTTAACAGTGTTGCAGATCTATTCAGATGTTTAAAATAATG | | |
| | | CTTAAATTCAAAGAGTTTTGCTTCTAGTGAACTGTAATCACTAGAAAATTTTGCCTCTAGGTATGGCTAAT | | |
| | | GTAACTTTACATTATGTAAGAAAAATTTTATTGTTTCTGCTTCTATACAAGAAGCCAAGAGTTTTAATCTTT | | |
| | | CAGTGTATATTGTTTCCTAAGTGAAAAGTATTTTATTAACTAATGCTTCTTAAAGTTTACCTTAAATCCTTGC | | |
| | | TCTCACCCAAAAGATTCAGAGACAATATCCTTTTATTACTTAGGGTTTTAGTTTACTACAAAAGTTTCTACA | | |
| | | AAAAATAAAGCTTTTATAATTGTTATTAATTGGTAATTAAAAATTGGTTGTGCCCAAAACAATTCTTTGGCC | | |
| | | AAAAAAAAAAACATTATTGTAAAGTCATTTTTCTCATCCTCCCAGCCAATCGTTGGCCCACGTGGGCCCAA | | |
| | | CTAGCTTCTGTGGGGCAGAGTCTTAAGACACAGTTTCCCCTGTTCCAGCACAGATGATCTAGTTGTGTGCT | | |
| | | GTAGATGGTGTTTTAAAATGCTGAACAATCAAACCTTAATTTGTATATTAATAGTCAATGCCATATCTCTGA | | |
| | | GCTCACAATTGCTTAAATTGTTCATCCCTCAGATACTATTAATTCTCAAATTTACAATTGCTTATGCATATTT | | |
| | | CTAGTTAATAAATAAATTATGCACATGTGACTCTTAATAACTTTACAAGCCTTCTAGTTACAACTGCTCCTT | | |
| | | AAGAAAATTGATTAAAAGTGCAATTAGTCACTGCCCCTTTACAGCCAAGTATTTAAAATGTTTTGTCAACT | | |
| | | AGTTATTAATTCAAAAGTTTAGGTATTGTAAAATTTTAAAACTTTAACTTCTTAAAAGACAAAAAAGAGAG | | |
| | | AAATTGTATCCCCGTGCATGCTATTTAGTgaacaaacgacccaacacccgtgcgttttattctgtctttttattgccgatccccc | | |
| | | ggccgctttacttgtacagctcgtccatgccgagagtgatcccggcggcggtcacgaactccagcaggaccatgtgatcgcgcttctcgttg | | |
| | | gggtctttgctcagggcggactgggtgctcaggtaagtatcaaggttacaagacaggtttaaggagaccaatagaaactgggcttgtcga | | |
| | | gacagagaagactcttgcgtttctgataggcacctattggtcttactgacatccactttgcctttctctccacaggtagtggttgtcgggca | | |
| | | gcagcacggggccgtcgccgatgggggtgttctgctggtagtggtcggcgagctgcacgctgccgtcctcgatgttgtggcggatcttgaag | | |
| | | ttcaccttgatgccgttcttctgcttgtcggccatgatatagacgttgtggctgttgtagttgtactccagcttgtgccccaggatgttgcc | | |
| | | gtcctccttgaagtcgatgcccttcagctcgatgcggttcaccagggtgtcgccctcgaacttcacctcggcgcgggtcttgtagttgccgt | | |
| | | cgtccttgaagaagatggtgcgctcctggacgtagccttcgggcatggcggacttgaagaagtcgtgctgcttcatgtggtcggggtagcgg | | |
| | | ctgaagcactgcacgccgtaggtcagggtggtcacgagggtgggccagggcacgggcagcttgccggtggtgcagatgaacttcagggtcag | | |
| | | cttgccgtaggtggcatcgccctcgccctcgccggacacgctgaacttgtggccgtttacgtcgccgtccagctcgaccaggatgggcacca | | |
| | | ccccggtgaacagctcctcgcccttgctcaccatggtggctttaccaacagtaccggaatgccaagcttgggtcctgtgttctggcggcaaa | | |
| | | cccgttgcgaaaaagaacgttcacggcgactactgcacttatatacggttctcccccaccctcgggaaaaaggcggagccagtacacgacat | | |
| | | cactttcccagtttaccccgcgccaccttctctaggcaccggatcaattgccgacccctccccccaacttctcggggactgtgggcgatgtg | | |
| | | cgctctgcccGCATTCCCATGCACACTATTAAAGTCTTACCTTTATTTTCAAAATCTAATATTTTAATGTCAAAGA | | |
| | | GTTTAATAAATGCTTTATAGTATCTTAAAGGGATCTACTTATTGGCTTATAGATTAATCCTAAAACAGCTAC | | |
| | | CTTATTAAAAAAGGGAAAAACAGGTTTTCTCCACAGAACGCTGCAGAAGCATATTAATAAATTCGTGTGA | | |
| | | CGAGCTGGTAGGTAAGTTGACTCATGTCCTGATTAAATTGACTAAAAAACTAAATTAAATTCATGTTTTAG | | |
| | | ATCCATCCTTACTTGTCATTTTTCCAGTTTAGACTAGCTTCTAGCCTTTTAACTTTATGACAATAGTACATCA | | |
| | | GAGACTGTATATTCAGACTTAGTAAAATTAGTCATTTAATAGAGTCATAATGATTTTTCTCCTTTCTTCAGT | | |
| | | GTGACCAGCCATTCTAACTCAATCTTAGACTGGTCCTAATATTCAGTCCAATGTTAGAGATTCCTATATTCT | | |
| | | AAATTACCTAGCAAGTTAATTAAAGAGCAATTGGTCACTTAATACCCCCTGACTAACAAATGGAAGGGTC | | |
| | | CAGATCCAGTTCTAATTTGGGGTAGGGACTCAGTTTGTGTTTTTTCACGAGATGAAGATGGAGCGCGGTG | | |
| | | GCTGCCAGAGAGATTAATTCGTCAGATGAACACAGATTCTGACTCTTCTGGTAAGTATCATTCTAAAGACT | | |
| | | AAAATTCCTTTTGTGCTTAAAATTCAGCTGAGAGCAACAGCTCTCAAAGGTGTTCTCCAGCTACTCTCTGA | | |
| | | ACCAGCTCCCGACAGGAGGCCGGAGACTAGCCTCAGCTTTACAATTTGCATTTGAATAAAGTACCTAGAC | | |
| | | TTCCCGGAAAGAAGTTCTGCTTTCCTACTTTCTCACTGTCTTTCAAGATTTTGTCTTTCAAGCAGGTAAATC | | |
| | | AACATTCCCGAGGCGGACCAGTAGATGTGCATCCCCGCCCCCCTAGAGCACACAGGTGGCAGCTGTTACC | | |
| | | CCCAGTCTCAGGACATTTCCAGCATGTGGCTTTCAGTCTGAGTTAAAAATTTAGGTTTACCTAGAGGGCTA | | |
| | | GAAGAGTAGATTTTTCTATATTAATAAAGATTGGTTTTTATTTTGATAGACAGGCTTAGCCCCTTAGCTGAC | | |
| | | CTCTGGCTTTTCACCCTTGCTGTTACTGCAAGGTGTCCTTAGCTCAATAGGCTGTGGAAAAAACAGGGATG | | |
| | | AGGAGGAACGACTTCCAGCTCCTATTTTACCCACAAATCGTGGTGTTATTAACGACATAATTCTTGCTTAG | | |
| | | GCTTTGCTAATTCTGAGGTTGATAATTCTCCTTTAGGAGCTGCACAGCACTCAGAACTGTGCATACTGGTT | | |
| | | TGTGATTGTACAAATTCAGTATGGGCACCGCTTGGTGCAGAGATACTTACTGCAAGGGAAGGTCCGGCTT | | |
| | | GACCATTTCTGAGTTTCCTGTGAGATAAACCCGGTTTGAAAGAGGTTGGTACCAAATTTTGGTTAAAAATA | | |
| | | AAAAATATTCTCCGGCTCTACCTCGCCTCCCCAAAAGGTACCAAGAGCCACATGTGTGGGTTTTACCCACG | | |
| | | GGAGGAATCGGGTCCATGTCCACCCAAGCCAAGGTTAAAAGCCCACTCATCTACGGATGAGAAAATCATT | | |
| | | TGATCACCTCAGTTAAGCGTTGCCTTATTTAACTTAATTAATAGGGGGGAGAGAGATTGGAGACGTACTA | | |
| | | TTGAAAGGGCAAGCCCTTCACTGCCTCCCACCCAAATAAAAAGGCCAATTGGCCTTGTACTACAAGAGCC | | |
| | | GGTCACTCCTTCTCCCTGTTTCCCACCTATCTTCCAAAAATGCTAAGGAATTCAACTTAGTGTTATTTTCACA | | |
| | | TCGTTCAGTCAAACTTAGCCAGAGTTCCAAACGCCCTACTTAAAATTCAACTAGAAAGTTACCTACCAAGT | | |
| | | ACTAATTAGCATTATAAAGTCAGAGTCTGCAGCTCCAGGCCTTTCAGTTGTTTACTAGAAAGGACAGTCTT | | |
| | | AAGCCAGATACAGTTTACCATAAGAAAAGTTAAAGATTCCCAGTGAAGCAAGTTTTTTCTTTAGCCCTAGA | | |
| | | TTCCAGGCAGAACTATTGAGCATAGATAATTTTCCCCCCTCAGGCCAGCTTTTTCTTTTTTTTTTTATTTTGT | | |
| | | TAATAACAGGGAGGAGATGTAGTCTCCCCTCCCCTAGCCTGAAACCTGCTTGCTCGGGGTGGAGCTTCCT | | |
| | | GCTCATTCGTTCTGCCACGCCCACTGCTGGAACCTGAGGAGCCACACACGTGCACCTTTCTACTGGACCAG | | |
| | | AGATTATTCGGCGGGAATCGGGTCCCCTCCCCCTTCCTTCATAACTGGTGTCGCAACAATAAAATTTGAGC | | |
| | | CTTGATCAGAGTAACTGTCTTGGCTACATTTCTTCTTTTGCCCCGTCTAGATTCCTCTCTTACAGCTCGAGC | | |
| | | GGCCTTCTCAGTCGAACCGTTCACGTTGCGAGCTGCTGGCGGCCGCAACAGGGGATCCAGACATGATAA | | |
| | | GATACATTGATGAGTTTGGACAAACCACAACTAGAATGCAGTGAAAAAAATGCTTTATTTGTGAAATTTG | | |
| | | TGATGCTATTGCTTTATTTGTAACCATTATAAGCTGCAATAAACAAGTTAACAACAACAATTGCATTCATTT | | |
| | | TATGTTTCAGGTTCAGGGGGAGGTGTGGGAGGTTTTTTCGGATCCTCTAGAGTCGACCTGCAGGCATGCA | | |
| | | AGCTTGGCGTAATCATGGTCATAGCTGTTTCCTGTGTGAAATTGTTATCCGCTCACAATTCCACACAACAT | | |
| | | ACGAGCCGGAAGCATAAAGTGTAAAGCCTGGGGTGCCTAATGAGTGAGCTAACTCACATTAATTGCGTT | | |
| | | GCGCTCACTGCCCGCTTTCCAGTCGGGAAACCTGTCGTGCCAGCTGCATTAATGAATCGGCCAACGCGCG | | |
| | | GGGAGAGGCGGTTTGCGTATTGGGCGCTCTTCCGCTTCCTCGCTCACTGACTCGCTGCGCTCGGTCGTTC | | |
| | | GGCTGCGGCGAGCGGTATCAGCTCACTCAAAGGCGGTAATACGGTTATCCACAGAATCAGGGGATAACG | | |
| | | CAGGAAAGAACATGTGAGCAAAAGGCCAGCAAAAGGCCAGGAACCGTAAAAAGGCCGCGTTGCTGGCG | | |
| | | TTTTTCCATAGGCTCCGCCCCCCTGACGAGCATCACAAAAATCGACGCTCAAGTCAGAGGTGGCGAAACC | | |
| | | CGACAGGACTATAAAGATACCAGGCGTTTCCCCCTGGAAGCTCCCTCGTGCGCTCTCCTGTTCCGACCCTG | | |
| | | CCGCTTACCGGATACCTGTCCGCCTTTCTCCCTTCGGGAAGCGTGGCGCTTTCTCATAGCTCACGCTGTAG | | |
| | | GTATCTCAGTTCGGTGTAGGTCGTTCGCTCCAAGCTGGGCTGTGTGCACGAACCCCCCGTTCAGCCCGAC | | |
| | | CGCTGCGCCTTATCCGGTAACTATCGTCTTGAGTCCAACCCGGTAAGACACGACTTATCGCCACTGGCAGC | | |
| | | AGCCACTGGTAACAGGATTAGCAGAGCGAGGTATGTAGGCGGTGCTACAGAGTTCTTGAAGTGGTGGCC | | |
| | | TAACTACGGCTACACTAGAAGGACAGTATTTGGTATCTGCGCTCTGCTGAAGCCAGTTACCTTCGGAAAA | | |
| | | AGAGTTGGTAGCTCTTGATCCGGCAAACAAACCACCGCTGGTAGCGGTGGTTTTTTTGTTTGCAAGCAGC | | |
| | | AGATTACGCGCAGAAAAAAAGGATCTCAAGAAGATCCTTTGATCTTTTCTACGGGGTCTGACGCTCAGTG | | |
| | | GAACGAAAACTCACGTTAAGGGATTTTGGTCATGAGATTATCAAAAAGGATCTTCACCTAGATCCTTTTAA | | |
| | | ATTAAAAATGAAGTTTTAAATCAATCTAAAGTATATATGAGTAAACTTGGTCTGACAGTTACCAATGCTTA | | |
| | | ATCAGTGAGGCACCTATCTCAGCGATCTGTCTATTTCGTTCATCCATAGTTGCCTGACTCCCCGTCGTGTAG | | |
| | | ATAACTACGATACGGGAGGGCTTACCATCTGGCCCCAGTGCTGCAATGATACCGCGAGACCCACGCTCAC | | |
| | | CGGCTCCAGATTTATCAGCAATAAACCAGCCAGCCGGAAGGGCCGAGCGCAGAAGTGGTCCTGCAACTT | | |
| | | TATCCGCCTCCATCCAGTCTATTAATTGTTGCCGGGAAGCTAGAGTAAGTAGTTCGCCAGTTAATAGTTTG | | |
| | | CGCAACGTTGTTGCCATTGCTACAGGCATCGTGGTGTCACGCTCGTCGTTTGGTATGGCTTCATTCAGCTC | | |
| | | CGGTTCCCAACGATCAAGGCGAGTTACATGATCCCCCATGTTGTGCAAAAAAGCGGTTAGCTCCTTCGGT | | |
| | | CCTCCGATCGTTGTCAGAAGTAAGTTGGCCGCAGTGTTATCACTCATGGTTATGGCAGCACTGCATAATTC | | |
| | | TCTTACTGTCATGCCATCCGTAAGATGCTTTTCTGTGACTGGTGAGTACTCAACCAAGTCATTCTGAGAAT | | |
| | | AGTGTATGCGGCGACCGAGTTGCTCTTGCCCGGCGTCAATACGGGATAATACCGCGCCACATAGCAGAA | | |
| | | CTTTAAAAGTGCTCATCATTGGAAAACGTTCTTCGGGGCGAAAACTCTCAAGGATCTTACCGCTGTTGAGA | | |
| | | TCCAGTTCGATGTAACCCACTCGTGCACCCAACTGATCTTCAGCATCTTTTACTTTCACCAGCGTTTCTGGG | | |
| | | TGAGCAAAAACAGGAAGGCAAAATGCCGCAAAAAAGGGAATAAGGGCGACACGGAAATGTTGAATACT | | |
| | | CATACTCTTCCTTTTTCAATATTATTGAAGCATTTATCAGGGTTATTGTCTCATGAGCGGATACATATTTGA | | |
| | | ATGTATTTAGAAAAATAAACAAATAGGGGTTCCGCGCACATTTCCCCGAAAAGTGCCACCTGACGTCTAA | | |
| | | GAAACCATTATTATCATGACATTAACCTATAAAAATAGGCGTATCACGAGGCCCTTTCGTCTCGCGCGTTT | | |
| | | CGGTGATGACGGTGAAAACCTCTGACACATGCAGCTCCCGGAGACGGTCACAGCTTGTCTGTAAGCGGA | | |
| | | TGCCGGGAGCAGACAAGCCCGTCAGGGCGCGTCAGCGGGTGTTGGCGGGTGTCGGGGCTGGCTTAACT | | |
| | | ATGCGGCATCAGAGCAGATTGTACTGAGAGTGCACCATATGCGGTGTGAAATACCGCACAGATGCGTAA | | |
| | | GGAGAAAATACCGCATCAGGCGCCATTCGCCATTCAGGCTGCGCAACTGTTGGGAAGGGCGATCGGTGC | | |
| | | GGGCCTCTTCGCTATTACGCCAGCTGGCGAAAGGGGGATGTGCTGCAAGGCGATTAAGTTGGGTAACGC | | |
| | | CAGGGTTTTCCCAGTCACGACGTTGTAAAACGACGGCCAGT | | |
| TEMPLATE_pCMV_ | PLV10070 | GAATTCGAGCTTGCATGCCTGCAGGTCGTTACATAACTTACGGTAAATGGCCCGCCTGGCTGACCGCCCA | 346 | N/A |
| R-U5-ETnII- | | ACGACCCCCGCCCATTGACGTCAATAATGACGTATGTTCCCATAGTAACGCCAATAGGGACTTTCCATTGA | | |
| B3_EF1-GFPai- | | CGTCAATGGGTGGAGTATTTACGGTAAACTGCCCACTTGGCAGTACATCAAGTGTATCATATGCCAAGTA | | |
| LTR_v2 | | CGCCCCCTATTGACGTCAATGACGGTAAATGGCCCGCCTGGCATTATGCCCAGTACATGACCTTATGGGA | | |
| | | CTTTCCTACTTGGCAGTACATCTACGTATTAGTCATCGCTATTACCATGGTGATGCGGTTTTGGCAGTACAT | | |
| | | CAATGGGCGTGGATAGCGGTTTGACTCACGGGGATTTCCAAGTCTCCACCCCATTGACGTCAATGGGAGT | | |
| | | TTGTTTTGGCACCAAAATCAACGGGACTTTCCAAAATGTCGTAACAACTCCGCCCCATTGACGCAAATGGG | | |
| | | CGGTAGGCGTGTACGGTGGGAGGTCTATATAAGCAGAGCTCGTTTAGTGAACCGTCAGCCTTGATCAGA | | |
| | | GTAACTGTCTTGGCTACATTTCTTCTTTTGCCCCGTCTAGATTCCTCTCTTACAGCTCGAGCGGCCTTCTCAG | | |
| | | TCGAACCGTTCACGTTGCGAGCTGCTGGCGGCCGCAACATTTTGGCGCCCGAACAGGGACCTGAAGAAT | | |
| | | GGCAGAGAGATGCTAAGAGGAACGCTGCATTGGAGCTCCACAGGAAAGGATCTTCGTATCGGACATCGG | | |
| | | AGCAACGGACAAGTACACATGCTAGCGCTAGCTTAAAATTTCAGTTTTGTAAAGTGTTGCTGAGGATGCG | | |
| | | GTAGGATACGAATTAAGCTTGAATCAGTGCTAACCCAACGCTGGTTCTGCTTGGGTCAGCAGCGTGTTAA | | |
| | | TCGGAACTAGAAACGGAAACAGGCAGGTTAGCCGCAGCTTTTTAGGAAGCTGCTTAGGTGAAAGAAGAA | | |
| | | AGGGTTTAAAGTCATGGATCAGGCGGTAGGCCGTAGCTCTCCGAAGCTACATGAGGTGTGAGAAAAGAA | | |
| | | AGGGTTTATTAAAAGGAATAGGCGGATTGCCCCAGTTAATAAAAAATACATCATAAGGGAGGAAAATGT | | |
| | | CCCAAAAAGCAGAGAGAAATATCTCTCTGGGCCTTATAGCAGGAGTACTCTGTTCCCCTTTGTGTCTTGTC | | |
| | | TAATGTCCGGTGCACCAATCTGTTCTCGTGTTCGATTCATGTATGTTCGTGTCCAGTCTGTATGAATGAAT | | |
| | | GTTCTATGTTTTGTGTTGGATAATAAAGATGGTATAAAAAACTTTATCTGCAAAGCCGAGAGCTGCCACGT | | |
| | | GTTTCAGCCAGGAATCAGACACGTGGCGAGAGGGCCCCTGCTGGAAAAACTGTTCGTTTTAGAAAATAA | | |
| | | GGGCGAGTGCACAGCCTCTAAGTTTCAGAGTAAAAAAGCTAATAAATGGTTCATGATTAATGTGTTTGAC | | |
| | | AATGGTAAAGTGTTTTTTATTTTATGATTGTAGCTACAAAAATTATTATTCTCTGATTGGTCTAAATGTAAC | | |
| | | TGCTTCATTTGGTTCTTTTTTATTGGTAATGTGCTCTAAGTGTTTTCACAATCAGCTCATAAGTTGTTGGTTA | | |
| | | AGATTAATAATTGTTACATTGCTACAGATGGTTAGTGTTAAATTTGATAACTCAAGTTTAGAGTCCTTCCAA | | |
| | | CACATGGCATAAGGCAGCCCAAGAGGCTGGGTCTCTAAAGATATTTCTAGTTTAGGTAATAATTAATATG | | |
| | | GTTCGTATCCTAAATAGTAAAATTTAAAATAAGATTTAAAGCAATGTCTCTTTATTAAAGCATTAAAGCTTG | | |
| | | CTTTAATAGGTATTCATAGGTATTAATTGACATCCAAACTTCGTAATACGATAGTAATGCTTCTAATATTGA | | |
| | | TTTTAAAGATAATAAATTGTTTTAAACTTGGGTTTTGCTTTCCCAAGGTTATAGGTATTATCCTAACCTTGC | | |
| | | ACAAAAAACTTAAAAATTATGGTTAAAACTGCTATTGTTCCATTGACTGCAGCTTGCAGTTTGATTTCAAAT | | |
| | | TTAAGATCTTTAATTCACCTGTATACTGTAATTAAGATAATTACAAGAGTAATCATCTTATGAGAGCGCTCA | | |
| | | TACAGCTCACTTCATACAAAACTGTGACAGAGTTATCTAGTTATGTTTGTCTTTGTGAATAGATTAGACAA | | |
| | | ACAGTTTGGTTATAGTTGCTGCCTGGCAGGAAACTGTAGTACAGGCAATTTTTTCACAACACTAAAGTTGT | | |
| | | GAAGAACGTTTTAACTGTAAGTCATCTTAAGAAAGAGTATCAAAATTTAGAGGCGTAGACAGTTATATTG | | |
| | | TTTCTCTAAAATCGGTCCTTATTACAGGAGGGCCAGGATGCTCAGATTAAAAAGTATTTTACGTTTGAGTC | | |
| | | AATGCAGGGCTCTGGGCAGCCCCAGAGCGGCTTGTGGCCTTTCTTTTGTTTTGCAAACAGTGCCTGAGAA | | |
| | | AGATTTTTCCCTGTGTTCAAGAAAAATTCTTTTTAACAGTGTTGCAGATCTATTCAGATGTTTAAAATAATG | | |
| | | CTTAAATTCAAAGAGTTTTGCTTCTAGTGAACTGTAATCACTAGAAAATTTTGCCTCTAGGTATGGCTAAT | | |
| | | GTAACTTTACATTATGTAAGAAAAATTTTATTGTTTCTGCTTCTATACAAGAAGCCAAGAGTTTTAATCTTT | | |
| | | CAGTGTATATTGTTTCCTAAGTGAAAAGTATTTTATTAACTAATGCTTCTTAAAGTTTACCTTAAATCCTTGC | | |
| | | TCTCACCCAAAAGATTCAGAGACAATATCCTTTTATTACTTAGGGTTTTAGTTTACTACAAAAGTTTCTACA | | |
| | | AAAAATAAAGCTTTTATAATTGTTATTAATTGGTAATTAAAAATTGGTTGTGCCCAAAACAATTCTTTGGCC | | |
| | | AAAAAAAAAAACATTATTGTAAAGTCATTTTTCTCATCCTCCCAGCCAATCGTTGGCCCACGTGGGCCCAA | | |
| | | CTAGCTTCTGTGGGGCAGAGTCTTAAGACACAGTTTCCCCTGTTCCAGCACAGATGATCTAGTTGTGTGCT | | |
| | | GTAGATGGTGTTTTAAAATGCTGAACAATCAAACCTTAATTTGTATATTAATAGTCAATGCCATATCTCTGA | | |
| | | GCTCACAATTGCTTAAATTGTTCATCCCTCAGATACTATTAATTCTCAAATTTACAATTGCTTATGCATATTT | | |
| | | CTAGTTAATAAATAAATTATGCACATGTGACTCTTAATAACTTTACAAGCCTTCTAGTTACAACTGCTCCTT | | |
| | | AAGAAAATTGATTAAAAGTGCAATTAGTCACTGCCCCTTTACAGCCAAGTATTTAAAATGTTTTGTCAACT | | |
| | | AGTTATTAATTCAAAAGTTTAGGTATTGTAAAATTTTAAAACTTTAACTTCTTAAAAGACAAAAAAGAGAG | | |
| | | AAATTGTATCCCCGTGCATGCTATTTAGTgaacaaacgacccaacacccgtgcgttttattctgtctttttattgccgatccccc | | |
| | | ggccgctttacttgtacagctcgtccatgccgagagtgatcccggcggcggtcacgaactccagcaggaccatgtgatcgcgcttctcgttg | | |
| | | gggtctttgctcagggcggactgggtgctcaggtaagtatcaaggttacaagacaggtttaaggagaccaatagaaactgggcttgtcga | | |
| | | gacagagaagactcttgcgtttctgataggcacctattggtcttactgacatccactttgcctttctctccacaggtagtggttgtcgggca | | |
| | | gcagcacggggccgtcgccgatgggggtgttctgctggtagtggtcggcgagctgcacgctgccgtcctcgatgttgtggcggatcttgaag | | |
| | | ttcaccttgatgccgttcttctgcttgtcggccatgatatagacgttgtggctgttgtagttgtactccagcttgtgccccaggatgttgcc | | |
| | | gtcctccttgaagtcgatgcccttcagctcgatgcggttcaccagggtgtcgccctcgaacttcacctcggcgcgggtcttgtagttgccgt | | |
| | | cgtccttgaagaagatggtgcgctcctggacgtagccttcgggcatggcggacttgaagaagtcgtgctgcttcatgtggtcggggtagcgg | | |
| | | ctgaagcactgcacgccgtaggtcagggtggtcacgagggtgggccagggcacgggcagcttgccggtggtgcagatgaacttcagggtcag | | |
| | | cttgccgtaggtggcatcgccctcgccctcgccggacacgctgaacttgtggccgtttacgtcgccgtccagctcgaccaggatgggcacca | | |
| | | ccccggtgaacagctcctcgcccttgctcaccatggtggctttaccaacagtaccggaatgccaagcttgggtcctgtgttctggggcaaac | | |
| | | ccgttgcgaaaaagaacgttcacggcgactactgcacttatatacggttctcccccaccctcgggaaaaaggcggagccagtacacgacatc | | |
| | | actttcccagtttaccccgcgccaccttctctaggcaccggatcaattgccgacccctccccccaacttctcggggactgtgggcgatgtgc | | |
| | | gctctgcccGCATTCCCATGCACACTATTAAAGTCTTACCTTTATTTTCAAAATCTAATATTTTAATGTCAAAGA | | |
| | | GTTTAATAAATGCTTTATAGTATCTTAAAGGGATCTACTTATTGGCTTATAGATTAATCCTAAAACAGCTAC | | |
| | | CTTATTAAAAAAGGGAAAAACAGGTTTTCTCCACAGAACGCTGCAGAAGCATATTAATAAATTCGTGTGA | | |
| | | CGAGCTGGTAGGTAAGTTGACTCATGTCCTGATTAAATTGACTAAAAAACTAAATTAAATTCATGTTTTAG | | |
| | | ATCCATCCTTACTTGTCATTTTTCCAGTTTAGACTAGCTTCTAGCCTTTTAACTTTATGACAATAGTACATCA | | |
| | | GAGACTGTATATTCAGACTTAGTAAAATTAGTCATTTAATAGAGTCATAATGATTTTTCTCCTTTCTTCAGT | | |
| | | GTGACCAGCCATTCTAACTCAATCTTAGACTGGTCCTAATATTCAGTCCAATGTTAGAGATTCCTATATTCT | | |
| | | AAATTACCTAGCAAGTTAATTAAAGAGCAATTGGTCACTTAATACCCCCTGACTAACAAATGGAAGGGTC | | |
| | | CAGATCCAGTTCTAATTTGGGGTAGGGACTCAGTTTGTGTTTTTTCACGAGATGAAGATGGAGCGCGGTG | | |
| | | GCTGCCAGAGAGATTAATTCGTCAGATGAACACAGATTCTGACTCTTCTGGTAAGTATCATTCTAAAGACT | | |
| | | AAAATTCCTTTTGTGCTTAAAATTCAGCTGAGAGCAACAGCTCTCAAAGGTGTTCTCCAGCTACTCTCTGA | | |
| | | ACCAGCTCCCGACAGGAGGCCGGAGACTAGCCTCAGCTTTACAATTTGCATTTGAATAAAGTACCTAGAC | | |
| | | TTCCCGGAAAGAAGTTCTGCTTTCCTACTTTCTCACTGTCTTTCAAGATTTTGTCTTTCAAGCAGGTAAATC | | |
| | | AACATTCCCGAGGCGGACCAGTAGATGTGCATCCCCGCCCCCCTAGAGCACACAGGTGGCAGCTGTTACC | | |
| | | CCCAGTCTCAGGACATTTCCAGCATGTGGCTTTCAGTCTGAGTTAAAAATTTAGGTTTACCTAGAGGGCTA | | |
| | | GAAGAGTAGATTTTTCTATATTAATAAAGATTGGTTTTTATTTTGATAGACAGGCTTAGCCCCTTAGCTGAC | | |
| | | CTCTGGCTTTTCACCCTTGCTGTTACTGCAAGGTGTCCTTAGCTCAATAGGCTGTGGAAAAAACAGGGATG | | |
| | | AGGAGGAACGACTTCCAGCTCCTATTTTACCCACAAATCGTGGTGTTATTAACGACATAATTCTTGCTTAG | | |
| | | GCTTTGCTAATTCTGAGGTTGATAATTCTCCTTTAGGAGCTGCACAGCACTCAGAACTGTGCATACTGGTT | | |
| | | TGTGATTGTACAAATTCAGTATGGGCACCGCTTGGTGCAGAGATACTTACTGCAAGGGAAGGTCCGGCTT | | |
| | | GACCATTTCTGAGTTTCCTGTGAGATAAACCCGGTTTGAAAGAGGTTGGTACCAAATTTTGGTTAAAAATA | | |
| | | AAAAATATTCTCCGGCTCTACCTCGCCTCCCCAAAAGGTACCAAGAGCCACATGTGTGGGTTTTACCCACG | | |
| | | GGAGGAATCGGGTCCATGTCCACCCAAGCCAAGGTTAAAAGCCCACTCATCTACGGATGAGAAAATCATT | | |
| | | TGATCACCTCAGTTAAGCGTTGCCTTATTTAACTTAATTAATAGGGGGGAGAGAGATTGGAGACGTACTA | | |
| | | TTGAAAGGGCAAGCCCTTCACTGCCTCCCACCCAAATAAAAAGGCCAATTGGCCTTGTACTACAAGAGCC | | |
| | | GGTCACTCCTTCTCCCTGTTTCCCACCTATCTTCCAAAAATGCTAAGGAATTCAACTTAGTGTTATTTTCACA | | |
| | | TCGTTCAGTCAAACTTAGCCAGAGTTCCAAACGCCCTACTTAAAATTCAACTAGAAAGTTACCTACCAAGT | | |
| | | ACTAATTAGCATTATAAAGTCAGAGTCTGCAGCTCCAGGCCTTTCAGTTGTTTACTAGAAAGGACAGTCTT | | |
| | | AAGCCAGATACAGTTTACCATAAGAAAAGTTAAAGATTCCCAGTGAAGCAAGTTTTTTCTTTAGCCCTAGA | | |
| | | TTCCAGGCAGAACTATTGAGCATAGATAATTTTCCCCCCTCAGGCCAGCTTTTTCTTTTTTTTTTTATTTTGT | | |
| | | TAATAACAGGGAGGAGATGTAGTCTCCCCTCCCCTAGCCTGAAACCTGCTTGCTCGGGGTGGAGCTTCCT | | |
| | | GCTCATTCGTTCTGCCACGCCCACTGCTGGAACCTGAGGAGCCACACACGTGCACCTTTCTACTGGACCAG | | |
| | | AGATTATTCGGCGGGAATCGGGTCCCCTCCCCCTTCCTTCATAACTGGTGTCGCAACAATAAAATTTGAGC | | |
| | | CTTGATCAGAGTAACTGTCTTGGCTACATTTCTTCTTTTGCCCCGTCTAGATTCCTCTCTTACAGCTCGAGC | | |
| | | GGCCTTCTCAGTCGAACCGTTCACGTTGCGAGCTGCTGGCGGCCGCAACAGGGGATCCAGACATGATAA | | |
| | | GATACATTGATGAGTTTGGACAAACCACAACTAGAATGCAGTGAAAAAAATGCTTTATTTGTGAAATTTG | | |
| | | TGATGCTATTGCTTTATTTGTAACCATTATAAGCTGCAATAAACAAGTTAACAACAACAATTGCATTCATTT | | |
| | | TATGTTTCAGGTTCAGGGGGAGGTGTGGGAGGTTTTTTCGGATCCTCTAGAGTCGACCTGCAGGCATGCA | | |
| | | AGCTTGGCGTAATCATGGTCATAGCTGTTTCCTGTGTGAAATTGTTATCCGCTCACAATTCCACACAACAT | | |
| | | ACGAGCCGGAAGCATAAAGTGTAAAGCCTGGGGTGCCTAATGAGTGAGCTAACTCACATTAATTGCGTT | | |
| | | GCGCTCACTGCCCGCTTTCCAGTCGGGAAACCTGTCGTGCCAGCTGCATTAATGAATCGGCCAACGCGCG | | |
| | | GGGAGAGGCGGTTTGCGTATTGGGCGCTCTTCCGCTTCCTCGCTCACTGACTCGCTGCGCTCGGTCGTTC | | |
| | | GGCTGCGGCGAGCGGTATCAGCTCACTCAAAGGCGGTAATACGGTTATCCACAGAATCAGGGGATAACG | | |
| | | CAGGAAAGAACATGTGAGCAAAAGGCCAGCAAAAGGCCAGGAACCGTAAAAAGGCCGCGTTGCTGGCG | | |
| | | TTTTTCCATAGGCTCCGCCCCCCTGACGAGCATCACAAAAATCGACGCTCAAGTCAGAGGTGGCGAAACC | | |
| | | CGACAGGACTATAAAGATACCAGGCGTTTCCCCCTGGAAGCTCCCTCGTGCGCTCTCCTGTTCCGACCCTG | | |
| | | CCGCTTACCGGATACCTGTCCGCCTTTCTCCCTTCGGGAAGCGTGGCGCTTTCTCATAGCTCACGCTGTAG | | |
| | | GTATCTCAGTTCGGTGTAGGTCGTTCGCTCCAAGCTGGGCTGTGTGCACGAACCCCCCGTTCAGCCCGAC | | |
| | | CGCTGCGCCTTATCCGGTAACTATCGTCTTGAGTCCAACCCGGTAAGACACGACTTATCGCCACTGGCAGC | | |
| | | AGCCACTGGTAACAGGATTAGCAGAGCGAGGTATGTAGGCGGTGCTACAGAGTTCTTGAAGTGGTGGCC | | |
| | | TAACTACGGCTACACTAGAAGGACAGTATTTGGTATCTGCGCTCTGCTGAAGCCAGTTACCTTCGGAAAA | | |
| | | AGAGTTGGTAGCTCTTGATCCGGCAAACAAACCACCGCTGGTAGCGGTGGTTTTTTTGTTTGCAAGCAGC | | |
| | | AGATTACGCGCAGAAAAAAAGGATCTCAAGAAGATCCTTTGATCTTTTCTACGGGGTCTGACGCTCAGTG | | |
| | | GAACGAAAACTCACGTTAAGGGATTTTGGTCATGAGATTATCAAAAAGGATCTTCACCTAGATCCTTTTAA | | |
| | | ATTAAAAATGAAGTTTTAAATCAATCTAAAGTATATATGAGTAAACTTGGTCTGACAGTTACCAATGCTTA | | |
| | | ATCAGTGAGGCACCTATCTCAGCGATCTGTCTATTTCGTTCATCCATAGTTGCCTGACTCCCCGTCGTGTAG | | |
| | | ATAACTACGATACGGGAGGGCTTACCATCTGGCCCCAGTGCTGCAATGATACCGCGAGACCCACGCTCAC | | |
| | | CGGCTCCAGATTTATCAGCAATAAACCAGCCAGCCGGAAGGGCCGAGCGCAGAAGTGGTCCTGCAACTT | | |
| | | TATCCGCCTCCATCCAGTCTATTAATTGTTGCCGGGAAGCTAGAGTAAGTAGTTCGCCAGTTAATAGTTTG | | |
| | | CGCAACGTTGTTGCCATTGCTACAGGCATCGTGGTGTCACGCTCGTCGTTTGGTATGGCTTCATTCAGCTC | | |
| | | CGGTTCCCAACGATCAAGGCGAGTTACATGATCCCCCATGTTGTGCAAAAAAGCGGTTAGCTCCTTCGGT | | |
| | | CCTCCGATCGTTGTCAGAAGTAAGTTGGCCGCAGTGTTATCACTCATGGTTATGGCAGCACTGCATAATTC | | |
| | | TCTTACTGTCATGCCATCCGTAAGATGCTTTTCTGTGACTGGTGAGTACTCAACCAAGTCATTCTGAGAAT | | |
| | | AGTGTATGCGGCGACCGAGTTGCTCTTGCCCGGCGTCAATACGGGATAATACCGCGCCACATAGCAGAA | | |
| | | CTTTAAAAGTGCTCATCATTGGAAAACGTTCTTCGGGGCGAAAACTCTCAAGGATCTTACCGCTGTTGAGA | | |
| | | TCCAGTTCGATGTAACCCACTCGTGCACCCAACTGATCTTCAGCATCTTTTACTTTCACCAGCGTTTCTGGG | | |
| | | TGAGCAAAAACAGGAAGGCAAAATGCCGCAAAAAAGGGAATAAGGGCGACACGGAAATGTTGAATACT | | |
| | | CATACTCTTCCTTTTTCAATATTATTGAAGCATTTATCAGGGTTATTGTCTCATGAGCGGATACATATTTGA | | |
| | | ATGTATTTAGAAAAATAAACAAATAGGGGTTCCGCGCACATTTCCCCGAAAAGTGCCACCTGACGTCTAA | | |
| | | GAAACCATTATTATCATGACATTAACCTATAAAAATAGGCGTATCACGAGGCCCTTTCGTCTCGCGCGTTT | | |
| | | CGGTGATGACGGTGAAAACCTCTGACACATGCAGCTCCCGGAGACGGTCACAGCTTGTCTGTAAGCGGA | | |
| | | TGCCGGGAGCAGACAAGCCCGTCAGGGCGCGTCAGCGGGTGTTGGCGGGTGTCGGGGCTGGCTTAACT | | |
| | | ATGCGGCATCAGAGCAGATTGTACTGAGAGTGCACCATATGCGGTGTGAAATACCGCACAGATGCGTAA | | |
| | | GGAGAAAATACCGCATCAGGCGCCATTCGCCATTCAGGCTGCGCAACTGTTGGGAAGGGCGATCGGTGC | | |
| | | GGGCCTCTTCGCTATTACGCCAGCTGGCGAAAGGGGGATGTGCTGCAAGGCGATTAAGTTGGGTAACGC | | |
| | | CAGGGTTTTCCCAGTCACGACGTTGTAAAACGACGGCCAGT | | |
| TEMPLATE_pCMV_ | PLV10071 | GAATTCGAGCTTGCATGCCTGCAGGTCGTTACATAACTTACGGTAAATGGCCCGCCTGGCTGACCGCCCA | 347 | N/A |
| R-U5-ETnII- | | ACGACCCCCGCCCATTGACGTCAATAATGACGTATGTTCCCATAGTAACGCCAATAGGGACTTTCCATTGA | | |
| B3_EF1-GFPai- | | CGTCAATGGGTGGAGTATTTACGGTAAACTGCCCACTTGGCAGTACATCAAGTGTATCATATGCCAAGTA | | |
| LTR_v3 | | CGCCCCCTATTGACGTCAATGACGGTAAATGGCCCGCCTGGCATTATGCCCAGTACATGACCTTATGGGA | | |
| | | CTTTCCTACTTGGCAGTACATCTACGTATTAGTCATCGCTATTACCATGGTGATGCGGTTTTGGCAGTACAT | | |
| | | CAATGGGCGTGGATAGCGGTTTGACTCACGGGGATTTCCAAGTCTCCACCCCATTGACGTCAATGGGAGT | | |
| | | TTGTTTTGGCACCAAAATCAACGGGACTTTCCAAAATGTCGTAACAACTCCGCCCCATTGACGCAAATGGG | | |
| | | CGGTAGGCGTGTACGGTGGGAGGTCTATATAAGCAGAGCTGAGCCTTGATCAGAGTAACTGTCTTGGCT | | |
| | | ACATTTCTTCTTTTGCCCCGTCTAGATTCCTCTCTTACAGCTCGAGCGGCCTTCTCAGTCGAACCGTTCACG | | |
| | | TTGCGAGCTGCTGGCGGCCGCAACATTTTGGCGCCCGAACAGGGACCTGAAGAATGGCAGAGAGATGCT | | |
| | | AAGAGGAACGCTGCATTGGAGCTCCACAGGAAAGGATCTTCGTATCGGACATCGGAGCAACGGACAAGT | | |
| | | ACACATGCTAGCGCTAGCTTAAAATTTCAGTTTTGTAAAGTGTTGCTGAGGATGCGGTAGGATACGAATT | | |
| | | AAGCTTGAATCAGTGCTAACCCAACGCTGGTTCTGCTTGGGTCAGCAGCGTGTTAATCGGAACTAGAAAC | | |
| | | GGAAACAGGCAGGTTAGCCGCAGCTTTTTAGGAAGCTGCTTAGGTGAAAGAAGAAAGGGTTTAAAGTCA | | |
| | | TGGATCAGGCGGTAGGCCGTAGCTCTCCGAAGCTACATGAGGTGTGAGAAAAGAAAGGGTTTATTAAAA | | |
| | | GGAATAGGCGGATTGCCCCAGTTAATAAAAAATACATCATAAGGGAGGAAAATGTCCCAAAAAGCAGAG | | |
| | | AGAAATATCTCTCTGGGCCTTATAGCAGGAGTACTCTGTTCCCCTTTGTGTCTTGTCTAATGTCCGGTGCAC | | |
| | | CAATCTGTTCTCGTGTTCGATTCATGTATGTTCGTGTCCAGTCTGTATGAATGAATGTTCTATGTTTTGTGT | | |
| | | TGGATAATAAAGATGGTATAAAAAACTTTATCTGCAAAGCCGAGAGCTGCCACGTGTTTCAGCCAGGAAT | | |
| | | CAGACACGTGGCGAGAGGGCCCCTGCTGGAAAAACTGTTCGTTTTAGAAAATAAGGGCGAGTGCACAGC | | |
| | | CTCTAAGTTTCAGAGTAAAAAAGCTAATAAATGGTTCATGATTAATGTGTTTGACAATGGTAAAGTGTTTT | | |
| | | TTATTTTATGATTGTAGCTACAAAAATTATTATTCTCTGATTGGTCTAAATGTAACTGCTTCATTTGGTTCTT | | |
| | | TTTTATTGGTAATGTGCTCTAAGTGTTTTCACAATCAGCTCATAAGTTGTTGGTTAAGATTAATAATTGTTA | | |
| | | CATTGCTACAGATGGTTAGTGTTAAATTTGATAACTCAAGTTTAGAGTCCTTCCAACACATGGCATAAGGC | | |
| | | AGCCCAAGAGGCTGGGTCTCTAAAGATATTTCTAGTTTAGGTAATAATTAATATGGTTCGTATCCTAAATA | | |
| | | GTAAAATTTAAAATAAGATTTAAAGCAATGTCTCTTTATTAAAGCATTAAAGCTTGCTTTAATAGGTATTCA | | |
| | | TAGGTATTAATTGACATCCAAACTTCGTAATACGATAGTAATGCTTCTAATATTGATTTTAAAGATAATAAA | | |
| | | TTGTTTTAAACTTGGGTTTTGCTTTCCCAAGGTTATAGGTATTATCCTAACCTTGCACAAAAAACTTAAAAA | | |
| | | TTATGGTTAAAACTGCTATTGTTCCATTGACTGCAGCTTGCAGTTTGATTTCAAATTTAAGATCTTTAATTC | | |
| | | ACCTGTATACTGTAATTAAGATAATTACAAGAGTAATCATCTTATGAGAGCGCTCATACAGCTCACTTCAT | | |
| | | ACAAAACTGTGACAGAGTTATCTAGTTATGTTTGTCTTTGTGAATAGATTAGACAAACAGTTTGGTTATAG | | |
| | | TTGCTGCCTGGCAGGAAACTGTAGTACAGGCAATTTTTTCACAACACTAAAGTTGTGAAGAACGTTTTAAC | | |
| | | TGTAAGTCATCTTAAGAAAGAGTATCAAAATTTAGAGGCGTAGACAGTTATATTGTTTCTCTAAAATCGGT | | |
| | | CCTTATTACAGGAGGGCCAGGATGCTCAGATTAAAAAGTATTTTACGTTTGAGTCAATGCAGGGCTCTGG | | |
| | | GCAGCCCCAGAGCGGCTTGTGGCCTTTCTTTTGTTTTGCAAACAGTGCCTGAGAAAGATTTTTCCCTGTGT | | |
| | | TCAAGAAAAATTCTTTTTAACAGTGTTGCAGATCTATTCAGATGTTTAAAATAATGCTTAAATTCAAAGAG | | |
| | | TTTTGCTTCTAGTGAACTGTAATCACTAGAAAATTTTGCCTCTAGGTATGGCTAATGTAACTTTACATTATG | | |
| | | TAAGAAAAATTTTATTGTTTCTGCTTCTATACAAGAAGCCAAGAGTTTTAATCTTTCAGTGTATATTGTTTC | | |
| | | CTAAGTGAAAAGTATTTTATTAACTAATGCTTCTTAAAGTTTACCTTAAATCCTTGCTCTCACCCAAAAGAT | | |
| | | TCAGAGACAATATCCTTTTATTACTTAGGGTTTTAGTTTACTACAAAAGTTTCTACAAAAAATAAAGCTTTT | | |
| | | ATAATTGTTATTAATTGGTAATTAAAAATTGGTTGTGCCCAAAACAATTCTTTGGCCAAAAAAAAAAACAT | | |
| | | TATTGTAAAGTCATTTTTCTCATCCTCCCAGCCAATCGTTGGCCCACGTGGGCCCAACTAGCTTCTGTGGG | | |
| | | GCAGAGTCTTAAGACACAGTTTCCCCTGTTCCAGCACAGATGATCTAGTTGTGTGCTGTAGATGGTGTTTT | | |
| | | AAAATGCTGAACAATCAAACCTTAATTTGTATATTAATAGTCAATGCCATATCTCTGAGCTCACAATTGCTT | | |
| | | AAATTGTTCATCCCTCAGATACTATTAATTCTCAAATTTACAATTGCTTATGCATATTTCTAGTTAATAAATA | | |
| | | AATTATGCACATGTGACTCTTAATAACTTTACAAGCCTTCTAGTTACAACTGCTCCTTAAGAAAATTGATTA | | |
| | | AAAGTGCAATTAGTCACTGCCCCTTTACAGCCAAGTATTTAAAATGTTTTGTCAACTAGTTATTAATTCAAA | | |
| | | AGTTTAGGTATTGTAAAATTTTAAAACTTTAACTTCTTAAAAGACAAAAAAGAGAGAAATTGTATCCCCGT | | |
| | | GCATGCTATTTAGTgaacaaacgacccaacacccgtgcgttttattctgtctttttattgccgatcccccggccgctttacttgtacagc | | |
| | | tcgtccatgccgagagtgatcccggcggcggtcacgaactccagcaggaccatgtgatcgcgcttctcgttggggtctttgctcagggcgga | | |
| | | ctgggtgctcaggtaagtatcaaggttacaagacaggtttaaggagaccaatagaaactgggcttgtcgagacagagaagactcttgcgt | | |
| | | ttctgataggcacctattggtcttactgacatccactttgcctttctctccacaggtagtggttgtcgggcagcagcacggggccgtcgccg | | |
| | | atgggggtgttctgctggtagtggtcggcgagctgcacgctgccgtcctcgatgttgtggcggatcttgaagttcaccttgatgccgttctt | | |
| | | ctgcttgtcggccatgatatagacgttgtggctgttgtagttgtactccagcttgtgccccaggatgttgccgtcctccttgaagtcgatgc | | |
| | | ccttcagctcgatgcggttcaccagggtgtcgccctcgaacttcacctcggcgcgggtcttgtagttgccgtcgtccttgaagaagatggtg | | |
| | | cgctcctggacgtagccttcgggcatggcggacttgaagaagtcgtgctgcttcatgtggtcggggtagcggctgaagcactgcacgccgta | | |
| | | ggtcagggtggtcacgaggggggccagggcacgggcagcttgccggtggtgcagatgaacttcagggtcagcttgccgtaggtggcatcgcc | | |
| | | ctcgccctcgccggacacgctgaacttgtggccgtttacgtcgccgtccagctcgaccaggatgggcaccaccccggtgaacagctcctcgc | | |
| | | ccttgctcaccatggtggctttaccaacagtaccggaatgccaagcttgggtcctgtgttctggcggcaaacccgttgcgaaaaagaacgtt | | |
| | | cacggcgactactgcacttatatacggttctcccccaccctcgggaaaaaggcggagccagtacacgacatcactttcccagtttaccccgc | | |
| | | gccaccttctctaggcaccggatcaattgccgacccctccccccaacttctcggggactgtgggcgatgtgcgctctgcccGCATTCCCAT | | |
| | | GCACACTATTAAAGTCTTACCTTTATTTTCAAAATCTAATATTTTAATGTCAAAGAGTTTAATAAATGCTTTA | | |
| | | TAGTATCTTAAAGGGATCTACTTATTGGCTTATAGATTAATCCTAAAACAGCTACCTTATTAAAAAAGGGA | | |
| | | AAAACAGGTTTTCTCCACAGAACGCTGCAGAAGCATATTAATAAATTCGTGTGACGAGCTGGTAGGTAAG | | |
| | | TTGACTCATGTCCTGATTAAATTGACTAAAAAACTAAATTAAATTCATGTTTTAGATCCATCCTTACTTGTC | | |
| | | ATTTTTCCAGTTTAGACTAGCTTCTAGCCTTTTAACTTTATGACAATAGTACATCAGAGACTGTATATTCAG | | |
| | | ACTTAGTAAAATTAGTCATTTAATAGAGTCATAATGATTTTTCTCCTTTCTTCAGTGTGACCAGCCATTCTA | | |
| | | ACTCAATCTTAGACTGGTCCTAATATTCAGTCCAATGTTAGAGATTCCTATATTCTAAATTACCTAGCAAGT | | |
| | | TAATTAAAGAGCAATTGGTCACTTAATACCCCCTGACTAACAAATGGAAGGGTCCAGATCCAGTTCTAATT | | |
| | | TGGGGTAGGGACTCAGTTTGTGTTTTTTCACGAGATGAAGATGGAGCGCGGTGGCTGCCAGAGAGATTA | | |
| | | ATTCGTCAGATGAACACAGATTCTGACTCTTCTGGTAAGTATCATTCTAAAGACTAAAATTCCTTTTGTGCT | | |
| | | TAAAATTCAGCTGAGAGCAACAGCTCTCAAAGGTGTTCTCCAGCTACTCTCTGAACCAGCTCCCGACAGG | | |
| | | AGGCCGGAGACTAGCCTCAGCTTTACAATTTGCATTTGAATAAAGTACCTAGACTTCCCGGAAAGAAGTT | | |
| | | CTGCTTTCCTACTTTCTCACTGTCTTTCAAGATTTTGTCTTTCAAGCAGGTAAATCAACATTCCCGAGGCGG | | |
| | | ACCAGTAGATGTGCATCCCCGCCCCCCTAGAGCACACAGGTGGCAGCTGTTACCCCCAGTCTCAGGACAT | | |
| | | TTCCAGCATGTGGCTTTCAGTCTGAGTTAAAAATTTAGGTTTACCTAGAGGGCTAGAAGAGTAGATTTTTC | | |
| | | TATATTAATAAAGATTGGTTTTTATTTTGATAGACAGGCTTAGCCCCTTAGCTGACCTCTGGCTTTTCACCC | | |
| | | TTGCTGTTACTGCAAGGTGTCCTTAGCTCAATAGGCTGTGGAAAAAACAGGGATGAGGAGGAACGACTT | | |
| | | CCAGCTCCTATTTTACCCACAAATCGTGGTGTTATTAACGACATAATTCTTGCTTAGGCTTTGCTAATTCTG | | |
| | | AGGTTGATAATTCTCCTTTAGGAGCTGCACAGCACTCAGAACTGTGCATACTGGTTTGTGATTGTACAAAT | | |
| | | TCAGTATGGGCACCGCTTGGTGCAGAGATACTTACTGCAAGGGAAGGTCCGGCTTGACCATTTCTGAGTT | | |
| | | TCCTGTGAGATAAACCCGGTTTGAAAGAGGTTGGTACCAAATTTTGGTTAAAAATAAAAAATATTCTCCG | | |
| | | GCTCTACCTCGCCTCCCCAAAAGGTACCAAGAGCCACATGTGTGGGTTTTACCCACGGGAGGAATCGGGT | | |
| | | CCATGTCCACCCAAGCCAAGGTTAAAAGCCCACTCATCTACGGATGAGAAAATCATTTGATCACCTCAGTT | | |
| | | AAGCGTTGCCTTATTTAACTTAATTAATAGGGGGGAGAGAGATTGGAGACGTACTATTGAAAGGGCAAG | | |
| | | CCCTTCACTGCCTCCCACCCAAATAAAAAGGCCAATTGGCCTTGTACTACAAGAGCCGGTCACTCCTTCTC | | |
| | | CCTGTTTCCCACCTATCTTCCAAAAATGCTAAGGAATTCAACTTAGTGTTATTTTCACATCGTTCAGTCAAA | | |
| | | CTTAGCCAGAGTTCCAAACGCCCTACTTAAAATTCAACTAGAAAGTTACCTACCAAGTACTAATTAGCATT | | |
| | | ATAAAGTCAGAGTCTGCAGCTCCAGGCCTTTCAGTTGTTTACTAGAAAGGACAGTCTTAAGCCAGATACA | | |
| | | GTTTACCATAAGAAAAGTTAAAGATTCCCAGTGAAGCAAGTTTTTTCTTTAGCCCTAGATTCCAGGCAGAA | | |
| | | CTATTGAGCATAGATAATTTTCCCCCCTCAGGCCAGCTTTTTCTTTTTTTTTTTATTTTGTTAATAACAGGGA | | |
| | | GGAGATGTAGTCTCCCCTCCCCTAGCCTGAAACCTGCTTGCTCGGGGTGGAGCTTCCTGCTCATTCGTTCT | | |
| | | GCCACGCCCACTGCTGGAACCTGAGGAGCCACACACGTGCACCTTTCTACTGGACCAGAGATTATTCGGC | | |
| | | GGGAATCGGGTCCCCTCCCCCTTCCTTCATAACTGGTGTCGCAACAATAAAATTTGAGCCTTGATCAGAGT | | |
| | | AACTGTCTTGGCTACATTTCTTCTTTTGCCCCGTCTAGATTCCTCTCTTACAGCTCGAGCGGCCTTCTCAGTC | | |
| | | GAACCGTTCACGTTGCGAGCTGCTGGCGGCCGCAACAGGGGATCCAGACATGATAAGATACATTGATGA | | |
| | | GTTTGGACAAACCACAACTAGAATGCAGTGAAAAAAATGCTTTATTTGTGAAATTTGTGATGCTATTGCTT | | |
| | | TATTTGTAACCATTATAAGCTGCAATAAACAAGTTAACAACAACAATTGCATTCATTTTATGTTTCAGGTTC | | |
| | | AGGGGGAGGTGTGGGAGGTTTTTTCGGATCCTCTAGAGTCGACCTGCAGGCATGCAAGCTTGGCGTAAT | | |
| | | CATGGTCATAGCTGTTTCCTGTGTGAAATTGTTATCCGCTCACAATTCCACACAACATACGAGCCGGAAGC | | |
| | | ATAAAGTGTAAAGCCTGGGGTGCCTAATGAGTGAGCTAACTCACATTAATTGCGTTGCGCTCACTGCCCG | | |
| | | CTTTCCAGTCGGGAAACCTGTCGTGCCAGCTGCATTAATGAATCGGCCAACGCGCGGGGAGAGGCGGTT | | |
| | | TGCGTATTGGGCGCTCTTCCGCTTCCTCGCTCACTGACTCGCTGCGCTCGGTCGTTCGGCTGCGGCGAGCG | | |
| | | GTATCAGCTCACTCAAAGGCGGTAATACGGTTATCCACAGAATCAGGGGATAACGCAGGAAAGAACATG | | |
| | | TGAGCAAAAGGCCAGCAAAAGGCCAGGAACCGTAAAAAGGCCGCGTTGCTGGCGTTTTTCCATAGGCTC | | |
| | | CGCCCCCCTGACGAGCATCACAAAAATCGACGCTCAAGTCAGAGGTGGCGAAACCCGACAGGACTATAA | | |
| | | AGATACCAGGCGTTTCCCCCTGGAAGCTCCCTCGTGCGCTCTCCTGTTCCGACCCTGCCGCTTACCGGATA | | |
| | | CCTGTCCGCCTTTCTCCCTTCGGGAAGCGTGGCGCTTTCTCATAGCTCACGCTGTAGGTATCTCAGTTCGG | | |
| | | TGTAGGTCGTTCGCTCCAAGCTGGGCTGTGTGCACGAACCCCCCGTTCAGCCCGACCGCTGCGCCTTATCC | | |
| | | GGTAACTATCGTCTTGAGTCCAACCCGGTAAGACACGACTTATCGCCACTGGCAGCAGCCACTGGTAACA | | |
| | | GGATTAGCAGAGCGAGGTATGTAGGCGGTGCTACAGAGTTCTTGAAGTGGTGGCCTAACTACGGCTACA | | |
| | | CTAGAAGGACAGTATTTGGTATCTGCGCTCTGCTGAAGCCAGTTACCTTCGGAAAAAGAGTTGGTAGCTC | | |
| | | TTGATCCGGCAAACAAACCACCGCTGGTAGCGGTGGTTTTTTTGTTTGCAAGCAGCAGATTACGCGCAGA | | |
| | | AAAAAAGGATCTCAAGAAGATCCTTTGATCTTTTCTACGGGGTCTGACGCTCAGTGGAACGAAAACTCAC | | |
| | | GTTAAGGGATTTTGGTCATGAGATTATCAAAAAGGATCTTCACCTAGATCCTTTTAAATTAAAAATGAAGT | | |
| | | TTTAAATCAATCTAAAGTATATATGAGTAAACTTGGTCTGACAGTTACCAATGCTTAATCAGTGAGGCACC | | |
| | | TATCTCAGCGATCTGTCTATTTCGTTCATCCATAGTTGCCTGACTCCCCGTCGTGTAGATAACTACGATACG | | |
| | | GGAGGGCTTACCATCTGGCCCCAGTGCTGCAATGATACCGCGAGACCCACGCTCACCGGCTCCAGATTTA | | |
| | | TCAGCAATAAACCAGCCAGCCGGAAGGGCCGAGCGCAGAAGTGGTCCTGCAACTTTATCCGCCTCCATCC | | |
| | | AGTCTATTAATTGTTGCCGGGAAGCTAGAGTAAGTAGTTCGCCAGTTAATAGTTTGCGCAACGTTGTTGCC | | |
| | | ATTGCTACAGGCATCGTGGTGTCACGCTCGTCGTTTGGTATGGCTTCATTCAGCTCCGGTTCCCAACGATC | | |
| | | AAGGCGAGTTACATGATCCCCCATGTTGTGCAAAAAAGCGGTTAGCTCCTTCGGTCCTCCGATCGTTGTCA | | |
| | | GAAGTAAGTTGGCCGCAGTGTTATCACTCATGGTTATGGCAGCACTGCATAATTCTCTTACTGTCATGCCA | | |
| | | TCCGTAAGATGCTTTTCTGTGACTGGTGAGTACTCAACCAAGTCATTCTGAGAATAGTGTATGCGGCGACC | | |
| | | GAGTTGCTCTTGCCCGGCGTCAATACGGGATAATACCGCGCCACATAGCAGAACTTTAAAAGTGCTCATC | | |
| | | ATTGGAAAACGTTCTTCGGGGCGAAAACTCTCAAGGATCTTACCGCTGTTGAGATCCAGTTCGATGTAAC | | |
| | | CCACTCGTGCACCCAACTGATCTTCAGCATCTTTTACTTTCACCAGCGTTTCTGGGTGAGCAAAAACAGGA | | |
| | | AGGCAAAATGCCGCAAAAAAGGGAATAAGGGCGACACGGAAATGTTGAATACTCATACTCTTCCTTTTTC | | |
| | | AATATTATTGAAGCATTTATCAGGGTTATTGTCTCATGAGCGGATACATATTTGAATGTATTTAGAAAAAT | | |
| | | AAACAAATAGGGGTTCCGCGCACATTTCCCCGAAAAGTGCCACCTGACGTCTAAGAAACCATTATTATCAT | | |
| | | GACATTAACCTATAAAAATAGGCGTATCACGAGGCCCTTTCGTCTCGCGCGTTTCGGTGATGACGGTGAA | | |
| | | AACCTCTGACACATGCAGCTCCCGGAGACGGTCACAGCTTGTCTGTAAGCGGATGCCGGGAGCAGACAA | | |
| | | GCCCGTCAGGGCGCGTCAGCGGGTGTTGGCGGGTGTCGGGGCTGGCTTAACTATGCGGCATCAGAGCA | | |
| | | GATTGTACTGAGAGTGCACCATATGCGGTGTGAAATACCGCACAGATGCGTAAGGAGAAAATACCGCAT | | |
| | | CAGGCGCCATTCGCCATTCAGGCTGCGCAACTGTTGGGAAGGGCGATCGGTGCGGGCCTCTTCGCTATTA | | |
| | | CGCCAGCTGGCGAAAGGGGGATGTGCTGCAAGGCGATTAAGTTGGGTAACGCCAGGGTTTTCCCAGTCA | | |
| | | CGACGTTGTAAAACGACGGCCAGT | | |

TABLE S4
IAP LTR retrotransposon driver and template construct elements

| | | | SEQ | | SEQ |
|---|---|---|---|---|---|
| | plasmid_ | | ID | | ID |
| name | id | nucleic_acid_sequence | NO: | protein_sequence | NO: |
| IAP- | N/A | TGTTGGGAGCCGCGCCCACATTCGCCGTTACAAGATGGCGCTGACAGC | 348 | N/A | |
| 92L23 | | TGTGTTCTAAGTGGTAAACAAATAATCTGCGCATGTGCCGAGGGTGGT | | | |
| U3 | | TCTTCACTCTATGTGCTCTGCCTTCCCCGTGACGTCAACTCGGCCGATG | | | |
| | | GGCTGCAGCCAATCAGGGAGTGACACGTCCTAGGCGAAGGAGAATTC | | | |
| | | TCTTTAATAGGGACGGGGTTTT | | | |
| IAP- | N/A | GTTTTCTCTCTCTCTTGC | 349 | N/A | |
| 92L23 R | | | | | |
| (full | | | | | |
| length | | | | | |
| for CMV | | | | | |
| v2 | | | | | |
| designs) | | | | | |
| IAP- | N/A | GGGACGGGGTTTCGTTTTCTCTCTCTCTTGC | 350 | | |
| 92L23 R | | | | | |
| (5′ | | | | | |
| extended | | | | | |
| into U3 | | | | | |
| for CMV | | | | | |
| v1 | | | | | |
| designs) | | | | | |
| IAP- | N/A | TTCTCGCTCGCTCTTGCTTCTTGCACTCTGGCTCCTGAAGATGTAAGCAA | 351 | N/A | |
| 92L23 | | TAAAGTTTTGCCGCAGAAGATTCTGGTCTGTGGTGTTCTTCCTGGCCGG | | | |
| U5 | | GCGTGAGAACGCGTCTAATAACA | | | |
| IAP- | N/A | TGTTGGGAGCCGCGCCCACATTCGCCGTTACAAGATGGCGCTGACAGC | 352 | N/A | |
| 92L23 | | TGTGTTCTAAGTGGTAAACAAATAATCTGCGCATGTGCCGAGGGTGGT | | | |
| 5′/3′ LTR | | TCTTCACTCTATGTGCTCTGCCTTCCCCGTGACGTCAACTCGGCCGATG | | | |
| | | GGCTGCAGCCAATCAGGGAGTGACACGTCCTAGGCGAAGGAGAATTC | | | |
| | | TCTTTAATAGGGACGGGGTTTTGTTTTCTCTCTCTCTTGCTTCTCGCTCG | | | |
| | | CTCTTGCTTCTTGCACTCTGGCTCCTGAAGATGTAAGCAATAAAGTTTT | | | |
| | | GCCGCAGAAGATTCTGGTCTGTGGTGTTCTTCCTGGCCGGGCGTGAGA | | | |
| | | ACGCGTCTAATAACA | | | |
| IAP- | N/A | GGTGCCGAATTCCGGGA | 353 | N/A | |
| 92L23 | | | | | |
| PBS | | | | | |
| IAP- | N/A | TAGCGACTAAACACATCA | 309 | N/A | |
| 92L23 | | | | | |
| PBS* | | | | | |
| IAP- | N/A | ATGAATTCAGAACTTTTCAGCTGGGGAACGAGAGTACCAGTGAGTACA | 354 | MNSELFSWGTRVPVSTALRGKSDLELSKEIQDSLSEVKWNM | 363 |
| 92L23 | | GCTTTACGAGGTAAGTCTGATCTTGAACTTTCTAAGGAAATTCAAGACA | | FGLEFFLVLGALLFLFTCYQVIKIGLKILEEIQDKLSEVKR | |
| gag | | GTCTATCAGAAGTAAAGTGGAATATGTTTGGCCTTGAATTTTTTCTGGT | | GERVGAKRKYGTQNKYTGLSKGLEPEEKLRLGRNTWREIRR | |
| | | GTTAGGAGCCCTTTTGTTCCTTTTCACATGTTATCAAGTGATTAAGATAG | | KRGKREKKKDQLAEVSRRYSSLDELRKPALSSSEADEEFSS | |
| | | GGCTGAAAATTCTAGAGGAAATTCAGGACAAGCTATCAGAAGTAAAGC | | EETDWEEEAAHYQPANWSRKKPKAAGEGQFADWPQGSRLQG | |
| | | GGGGAGAGAGAGTAGGAGCAAAGAGAAAATATGGTACACAAAATAA | | PPYAESPPCVVRQQCAERQCAERQCADSFIPREEQRKIQQA | |
| | | GTATACAGGCCTTTCCAAGGGTCTTGAACCCGAGGAAAAGTTAAGGTT | | FPVFEGAEGGRVHAPVEYLQIKEIAESVRKYGTNANFTLVQ | |
| | | AGGTAGGAATACCTGGAGAGAGATTAGAAGAAAAAGAGGAAAAAGG | | LDRLAGMALTPADWQTVVKAALPSMGKYMEWRALWHEAAQA | |
| | | GAAAAGAAGAAAGATCAATTAGCGGAGGTCTCTAGGAGATACTCGTC | | QARANAAALTPEQRDWTFDLLTGQGAYSADQTNYHWGAYAQ | |
| | | ACTAGATGAGCTCAGGAAGCCAGCTCTTAGTAGTTCTGAAGCAGATGA | | ISSTAIRAWKALSRAGETTGQLTKIIQGPQESFSDFVARMT | |
| | | AGAATTCTCCTCTGAGGAAACAGACTGGGAGGAAGAAGCAGCCCATTA | | EAAERIFGESEQAAPLIEQLIYEQATKECRAAIAPRKNKGL | |
| | | CCAGCCAGCTAATTGGTCAAGAAAAAAGCCAAAAGCGGCTGGCGAAG | | QDWLRVCRELGGPLSNAGLAAAILQSQNRSMGRNNQRTCFN | |
| | | GCCAGTTTGCTGATTGGCCTCAGGGCAGTCGGCTTCAAGGTCCGCCCT | | CGKPGHFKKDCRAPDKQGGTLTLCSKCGKGYHRADQCRSVR | |
| | | ATGCGGAGTCCCCGCCCTGCGTAGTGCGTCAGCAATGCGCAGAGAGG | | DIKGRVLPPPDSQSADVPKNGSSGPRSQGPQRYGNRFVRTQ | |
| | | CAGTGCGCAGAGAGGCAGTGCGCAGACTCATTCATTCCCAGAGAGGA | | EAVREATQEDPQGWTCVPPPTSY* | |
| | | ACAAAGGAAAATACAACAGGCATTTCCGGTCTTTGAAGGAGCCGAGG | | | |
| | | GTGGGCGTGTCCACGCTCCGGTAGAATACTTACAAATTAAAGAAATTG | | | |
| | | CCGAGTCGGTCCGTAAATATGGAACCAATGCTAATTTTACCTTGGTGCA | | | |
| | | GTTAGACAGGCTCGCCGGCATGGCACTAACTCCTGCTGACTGGCAAAC | | | |
| | | GGTTGTAAAAGCCGCTCTCCCTAGTATGGGCAAATATATGGAATGGAG | | | |
| | | AGCGCTTTGGCACGAAGCTGCACAAGCGCAGGCCCGAGCAAACGCAG | | | |
| | | CTGCTTTGACTCCAGAGCAGAGAGATTGGACTTTTGACTTGTTAACGG | | | |
| | | GTCAGGGAGCTTATTCTGCTGATCAGACAAACTACCATTGGGGAGCTT | | | |
| | | ATGCCCAGATTTCTTCCACGGCTATTAGGGCCTGGAAGGCGCTCTCTCG | | | |
| | | AGCAGGTGAAACCACTGGTCAGTTAACAAAGATAATCCAGGGACCTCA | | | |
| | | GGAATCCTTCTCAGATTTTGTGGCCAGAATGACAGAGGCAGCAGAGCG | | | |
| | | TATTTTTGGAGAGTCAGAGCAAGCTGCGCCTCTGATAGAACAGCTAAT | | | |
| | | CTATGAGCAAGCCACAAAGGAGTGCCGAGCGGCCATAGCCCCAAGAA | | | |
| | | AGAACAAAGGCTTACAAGACTGGCTCAGGGTCTGTCGAGAGCTTGGG | | | |
| | | GGACCTCTCAGCAATGCAGGTTTAGCGGCTGCCATCCTTCAATCCCAAA | | | |
| | | ACCGCTCCATGGGCAGAAATAATCAGAGGACATGTTTTAACTGCGGAA | | | |
| | | AGCCTGGGCATTTTAAGAAAGATTGCAGAGCTCCAGATAAACAGGGA | | | |
| | | GGGACTCTCACTCTTTGCTCTAAGTGTGGCAAGGGTTATCATAGAGCTG | | | |
| | | ACCAGTGTCGCTCTGTGAGGGATATAAAGGGCAGAGTCCTTCCCCCAC | | | |
| | | CTGATAGTCAATCAGCTGATGTGCCAAAAAACGGGTCATCGGGCCCTC | | | |
| | | GGTCCCAGGGCCCTCAAAGATATGGGAACCGGTTTGTCAGGACCCAG | | | |
| | | GAAGCAGTCAGAGAGGCGACCCAGGAAGACCCACAAGGGTGGACCTG | | | |
| | | CGTGCCGCCTCCGACTTCCTACTAA | | | |
| IAP- | N/A | AATGCCTCAAATGAGTATTCAGCCGGTGCCGGTGGAGCCTATACCATC | 355 | MPQMSIQPVPVEPIPSLPPGTMGLILGRGSLTLQGLVVHPG | 364 |
| 92L23 | | CTTGCCCCCGGGAACCATGGGCCTTATTCTCGGCCGGGGTTCACTCACC | | VMDCQHSPEIQVLCSSPKGVFSISKGDRIAQLLLLPDNTRE | |
| pro | | TTGCAGGGCTTAGTAGTCCACCCTGGAGTTATGGATTGTCAACATTCCC | | KFAGPEIKKMGSSGNDSAYLVVSLNDRPKLRLKINGKEFEG | |
| | | CTGAAATACAGGTCCTGTGCTCAAGCCCTAAAGGCGTTTTTTCTATTAG | | ILDTGADKSIISTHWWPKAWPTTESSHSLQGLGYQSCPTIS | |
| | | TAAAGGAGATAGGATAGCTCAGCTGCTGCTCCTCCCTGATAATACCAG | | SIALTWESSEGQQGKFIPYVLPLPVNLWGRDIMQHLGLILS | |
| | | GGAGAAATTTGCAGGACCTGAGATAAAGAAAATGGGCTCCTCAGGAA | | NENAPSGGYSAKAKNIMAKMGYKEGKGLGHQEQGRIEPISP | |
| | | ATGATTCTGCCTATTTGGTTGTATCTTTAAATGATAGACCTAAGCTCCGC | | NGNQDRQGLGFP* | |
| | | CTTAAGATTAACGGAAAAGAGTTTGAAGGCATCCTTGATACCGGAGCA | | | |
| | | GATAAAAGTATAATTTCTACACATTGGTGGCCCAAAGCATGGCCCACCA | | | |
| | | CAGAGTCATCTCATTCATTACAGGGCCTAGGATATCAATCATGTCCCAC | | | |
| | | TATAAGCTCCATTGCCTTGACGTGGGAATCCTCTGAAGGACAGCAAGG | | | |
| | | GAAATTCATACCTTATGTGCTCCCACTCCCGGTTAACCTCTGGGGAAGG | | | |
| | | GATATTATGCAGCATTTGGGCCTTATTTTGTCCAATGAAAACGCCCCAT | | | |
| | | CAGGAGGGTATTCAGCTAAAGCAAAAAATATCATGGCAAAGATGGGTT | | | |
| | | ATAAAGAAGGAAAAGGGTTAGGACATCAAGAACAGGGAAGGATAGA | | | |
| | | GCCCATCTCACCTAATGGAAACCAAGACAGACAGGGTCTGGGTTTTCC | | | |
| | | TTAG | | | |
| IAP- | N/A | TGGAAACCAAGACAGACAGGGTCTGGGTTTTCCTTAGCGGCCATTGGG | 356 | WKPRQTGSGFSLAAIGAARPIPWKTGDPVWVPQWHLSSEKL | 365 |
| 92L23 | | GCAGCACGACCCATACCATGGAAAACAGGGGACCCAGTGTGGGTTCCT | | EAVIQLVEEQLKLGHIEPSTSPWNTPIFVIKKKSGKWRLLH | |
| pol | | CAATGGCACCTATCCTCTGAAAAACTAGAAGCTGTGATTCAACTGGTA | | DLRAINEQMNLFGPVQRGLPVLSALPRGWNLIIIDIKDCFF | |
| | | GAGGAACAATTAAAACTAGGCCATATTGAACCCTCTACCTCACCTTGGA | | SIPLCPRDRPRFAFTIPSINHMEPDKRYQWKVLPQGMSNSP | |
| | | ATACTCCAATTTTTGTAATTAAGAAAAAGTCAGGAAAGTGGAGACTGC | | TMCQLYVQEALLPVREQFPSLILLLYMDDILLCHKDLTMLQ | |
| | | TCCATGACCTCAGAGCCATTAATGAGCAAATGAACTTATTTGGCCCAGT | | KAYPFLLKTLSQWGLQIATEKVQISDTGQFLGSVVSPDKIV | |
| | | ACAGAGGGGTCTCCCTGTACTTTCCGCCTTACCACGTGGCTGGAATTTA | | PQKVEIRRDHLHTLNDFQKLLGDINWLRPFLKIPSAELRPL | |
| | | ATTATTATAGATATTAAAGATTGTTTCTTTTCTATACCTTTGTGTCCAAG | | FSILEGDPHISSPRTLTLAANQALQKVEKALQNAQLQRIED | |
| | | GGATAGGCCCAGATTTGCCTTTACCATCCCCTCTATTAATCACATGGAA | | SQPFSLCVFKTAQLPTAVLWQNGPLLWIHPNVSPAKIIDWY | |
| | | CCTGATAAGAGGTATCAATGGAAGGTCTTACCACAGGGAATGTCCAAT | | PDAIAQLALKGLKAAITHFGQSPYLLIVPYTAAQVQTLAAT | |
| | | AGTCCTACAATGTGCCAACTTTATGTGCAAGAAGCTCTTTTGCCAGTGA | | SNDWAVLVTSFSGKIDNHYPKHPILQFAQNQSVVFPQITVR | |
| | | GGGAACAATTCCCCTCTTTAATTTTGCTCCTTTACATGGATGACATCCTC | | NPLKNGIVVYTDGSKTGIGAYVANGKVVSKQYNENSPQVVE | |
| | | CTGTGCCATAAAGACCTTACCATGCTACAAAAGGCATATCCTTTTCTACT | | CLVVLEVLKTFLEPLNIVSDSCYVVNAVNLLEVAGVIKPSS | |
| | | TAAAACTTTAAGTCAGTGGGGTTTACAGATAGCCACAGAAAAGGTCCA | | RVANIFQQIQLVLLSRRFPVYITHVRAHSGLPGPMALGNNL | |
| | | AATTTCTGATACAGGACAATTCTTGGGCTCTGTGGTGTCCCCAGATAAG | | ADKATKVVAAALSSPVEAARNFHNNFHVTAETLRSRFSLTR | |
| | | ATTGTGCCCCAAAAGGTAGAGATAAGAAGAGATCACCTCCATACCTTA | | KEARDIVTQCQSCCEFLPVPHVGINPRGIRPLQVWQMDVTH | |
| | | AATGATTTTCAAAAGCTGTTGGGAGATATTAATTGGCTCAGACCTTTTT | | VSSFGKLQYLHVSIDTCSGIMFASPLTGEKASHVIQHCLEA | |
| | | TAAAGATTCCTTCCGCTGAGTTAAGGCCTTTGTTTAGTATTTTAGAAGG | | WSAWGKPRLLKTDNGPAYTSQKFQQFCRQMDVTHLTGLPYN | |
| | | AGATCCTCATATCTCCTCCCCTAGGACTCTTACTCTAGCTGCTAACCAGG | | PQGQGIVERAHRTLKTYLIKQKRGTFEETVPRAPRVSVSMA | |
| | | CCTTACAAAAGGTGGAAAAAGCCTTACAGAATGCACAATTACAACGTA | | LFTLNFLNIDAHGHTAAERHCTEPDRPNEMVKWKNVLDNKW | |
| | | TTGAGGATTCGCAGCCTTTCAGTTTGTGTGTCTTTAAGACAGCACAATT | | YGPDPILIRSRGAICVFPQNENNPFWIPERLTRKIQTDQGN | |
| | | GCCAACTGCAGTTTTGTGGCAGAATGGGCCATTGTTGTGGATCCATCC | | TNVPRLGDVQGVNNKKRAALGDNVDISTPNDGDV* | |
| | | AAACGTATCCCCAGCTAAAATAATAGATTGGTATCCTGATGCAATTGCA | | | |
| | | CAGCTTGCCCTTAAAGGTCTAAAAGCAGCAATCACCCACTTTGGGCAAA | | | |
| | | GTCCATATCTTTTAATTGTACCTTATACCGCTGCACAGGTTCAAACCTTG | | | |
| | | GCAGCCACATCTAATGATTGGGCAGTTTTAGTTACCTCCTTTTCAGGAA | | | |
| | | AAATAGATAACCATTATCCAAAACATCCAATCTTACAGTTTGCCCAAAA | | | |
| | | TCAATCTGTTGTGTTTCCACAAATAACAGTAAGAAACCCACTTAAAAAT | | | |
| | | GGGATTGTGGTATATACTGATGGATCAAAAACTGGCATAGGTGCCTAT | | | |
| | | GTGGCTAATGGTAAAGTGGTATCCAAACAATATAATGAAAATTCACCTC | | | |
| | | AAGTGGTAGAATGTTTAGTGGTCTTAGAAGTTTTAAAAACCTTTTTAGA | | | |
| | | ACCCCTTAATATTGTGTCAGATTCCTGTTATGTGGTTAATGCAGTAAATC | | | |
| | | TTTTAGAAGTGGCTGGAGTGATTAAGCCTTCCAGTAGAGTTGCCAATAT | | | |
| | | TTTTCAGCAGATACAATTAGTTTTGTTATCTAGAAGATTTCCTGTTTATA | | | |
| | | TTACTCATGTTAGAGCCCATTCAGGCCTACCTGGCCCCATGGCTCTGGG | | | |
| | | AAATAATTTGGCAGATAAGGCCACTAAAGTGGTGGCTGCTGCCCTATC | | | |
| | | ATCCCCGGTAGAGGCTGCAAGAAATTTTCATAACAATTTTCATGTGACG | | | |
| | | GCTGAAACATTACGCAGTCGTTTCTCCTTGACAAGAAAAGAAGCCCGT | | | |
| | | GACATTGTTACTCAATGTCAAAGCTGCTGTGAGTTCTTGCCAGTTCCTC | | | |
| | | ATGTGGGAATTAACCCACGCGGTATTCGACCTCTACAGGTCTGGCAAA | | | |
| | | TGGATGTTACACATGTTTCTTCCTTTGGAAAACTTCAATATCTCCATGTG | | | |
| | | TCCATTGACACATGTTCTGGCATCATGTTTGCTTCTCCATTAACCGGAGA | | | |
| | | AAAAGCCTCACATGTGATTCAACATTGCCTTGAGGCATGGAGTGCTTG | | | |
| | | GGGGAAACCCAGACTCCTTAAGACTGATAATGGACCAGCTTATACGTC | | | |
| | | TCAAAAATTCCAACAGTTCTGCCGTCAGATGGACGTGACCCACCTGACT | | | |
| | | GGACTTCCATACAACCCTCAAGGACAGGGTATTGTTGAGCGTGCGCAT | | | |
| | | CGCACCCTCAAAACCTATCTTATAAAACAGAAGAGGGGAACTTTTGAG | | | |
| | | GAGACTGTACCCCGAGCACCAAGAGTGTCGGTGTCTATGGCACTCTTT | | | |
| | | ACACTCAATTTTTTAAATATTGATGCTCATGGCCATACTGCGGCTGAAC | | | |
| | | GTCATTGTACAGAGCCAGATAGGCCCAATGAGATGGTTAAATGGAAAA | | | |
| | | ATGTCCTTGATAATAAATGGTATGGCCCGGATCCTATTTTGATAAGATC | | | |
| | | CAGGGGAGCTATCTGTGTTTTCCCACAGAATGAAAACAACCCATTTTGG | | | |
| | | ATACCAGAAAGACTCACCCGAAAAATCCAGACTGACCAAGGAAATACT | | | |
| | | AATGTCCCTCGTCTTGGTGATGTCCAGGGCGTCAATAATAAAAAGAGA | | | |
| | | GCAGCGTTGGGGGATAATGTCGACATTTCCACTCCCAATGACGGTGAT | | | |
| | | GTATAA | | | |
| CIS_pCMV_ | PLV10021 | GAATTCGAGCTTGCATGCCTGCAGGTCGTTACATAACTTACGGTAAATG | 357 | N/A | |
| R-U5- | | GCCCGCCTGGCTGACCGCCCAACGACCCCCGCCCATTGACGTCAATAAT | | | |
| IAP- | | GACGTATGTTCCCATAGTAACGCCAATAGGGACTTTCCATTGACGTCAA | | | |
| 92L23- | | TGGGTGGAGTATTTACGGTAAACTGCCCACTTGGCAGTACATCAAGTG | | | |
| delPol_ | | TATCATATGCCAAGTACGCCCCCTATTGACGTCAATGACGGTAAATGGC | | | |
| EF1- | | CCGCCTGGCATTATGCCCAGTACATGACCTTATGGGACTTTCCTACTTG | | | |
| GFPai- | | GCAGTACATCTACGTATTAGTCATCGCTATTACCATGGTGATGCGGTTT | | | |
| LTR_v1 | | TGGCAGTACATCAATGGGCGTGGATAGCGGTTTGACTCACGGGGATTT | | | |
| | | CCAAGTCTCCACCCCATTGACGTCAATGGGAGTTTGTTTTGGCACCAAA | | | |
| | | ATCAACGGGACTTTCCAAAATGTCGTAACAACTCCGCCCCATTGACGCA | | | |
| | | AATGGGCGGTAGGCGTGTACGGTGGGAGGTCTATATAAGCAGAGCTC | | | |
| | | GTTTAGTGAACCGTCAGGGACGGGGTTTCGTTTTCTCTCTCTCTTGCTTC | | | |
| | | TCGCTCGCTCTTGCTTCTTGCACTCTGGCTCCTGAAGATGTAAGCAATA | | | |
| | | AAGTTTTGCCGCAGAAGATTCTGGTCTGTGGTGTTCTTCCTGGCCGGGC | | | |
| | | GTGAGAACGCGTCTAATAACAATTGGTGCCGAATTCCGGGACGAGAAA | | | |
| | | AAAACTCGGGACTGGCGCAAGGAAGATCCCTCATTCCAGAACCAGAAC | | | |
| | | TGCGGGTCGCGGTAATAAAGGTTCCCGTAAAGCAGACTGTTAAGAAG | | | |
| | | GATTCAACTGTATGAATTCAGAACTTTTCAGCTGGGGAACGAGAGTAC | | | |
| | | CAGTGAGTACAGCTTTACGAGGTAAGTCTGATCTTGAACTTTCTAAGGA | | | |
| | | AATTCAAGACAGTCTATCAGAAGTAAAGTGGAATATGTTTGGCCTTGA | | | |
| | | ATTTTTTCTGGTGTTAGGAGCCCTTTTGTTCCTTTTCACATGTTATCAAG | | | |
| | | TGATTAAGATAGGGCTGAAAATTCTAGAGGAAATTCAGGACAAGCTAT | | | |
| | | CAGAAGTAAAGCGGGGAGAGAGAGTAGGAGCAAAGAGAAAATATGG | | | |
| | | TACACAAAATAAGTATACAGGCCTTTCCAAGGGTCTTGAACCCGAGGA | | | |
| | | AAAGTTAAGGTTAGGTAGGAATACCTGGAGAGAGATTAGAAGAAAAA | | | |
| | | GAGGAAAAAGGGAAAAGAAGAAAGATCAATTAGCGGAGGTCTCTAG | | | |
| | | GAGATACTCGTCACTAGATGAGCTCAGGAAGCCAGCTCTTAGTAGTTC | | | |
| | | TGAAGCAGATGAAGAATTCTCCTCTGAGGAAACAGACTGGGAGGAAG | | | |
| | | AAGCAGCCCATTACCAGCCAGCTAATTGGTCAAGAAAAAAGCCAAAAG | | | |
| | | CGGCTGGCGAAGGCCAGTTTGCTGATTGGCCTCAGGGCAGTCGGCTTC | | | |
| | | AAGGTCCGCCCTATGCGGAGTCCCCGCCCTGCGTAGTGCGTCAGCAAT | | | |
| | | GCGCAGAGAGGCAGTGCGCAGAGAGGCAGTGCGCAGACTCATTCATT | | | |
| | | CCCAGAGAGGAACAAAGGAAAATACAACAGGCATTTCCGGTCTTTGAA | | | |
| | | GGAGCCGAGGGTGGGCGTGTCCACGCTCCGGTAGAATACTTACAAATT | | | |
| | | AAAGAAATTGCCGAGTCGGTCCGTAAATATGGAACCAATGCTAATTTT | | | |
| | | ACCTTGGTGCAGTTAGACAGGCTCGCCGGCATGGCACTAACTCCTGCT | | | |
| | | GACTGGCAAACGGTTGTAAAAGCCGCTCTCCCTAGTATGGGCAAATAT | | | |
| | | ATGGAATGGAGAGCGCTTTGGCACGAAGCTGCACAAGCGCAGGCCCG | | | |
| | | AGCAAACGCAGCTGCTTTGACTCCAGAGCAGAGAGATTGGACTTTTGA | | | |
| | | CTTGTTAACGGGTCAGGGAGCTTATTCTGCTGATCAGACAAACTACCAT | | | |
| | | TGGGGAGCTTATGCCCAGATTTCTTCCACGGCTATTAGGGCCTGGAAG | | | |
| | | GCGCTCTCTCGAGCAGGTGAAACCACTGGTCAGTTAACAAAGATAATC | | | |
| | | CAGGGACCTCAGGAATCCTTCTCAGATTTTGTGGCCAGAATGACAGAG | | | |
| | | GCAGCAGAGCGTATTTTTGGAGAGTCAGAGCAAGCTGCGCCTCTGATA | | | |
| | | GAACAGCTAATCTATGAGCAAGCCACAAAGGAGTGCCGAGCGGCCAT | | | |
| | | AGCCCCAAGAAAGAACAAAGGCTTACAAGACTGGCTCAGGGTCTGTCG | | | |
| | | AGAGCTTGGGGGACCTCTCAGCAATGCAGGTTTAGCGGCTGCCATCCT | | | |
| | | TCAATCCCAAAACCGCTCCATGGGCAGAAATAATCAGAGGACATGTTTT | | | |
| | | AACTGCGGAAAGCCTGGGCATTTTAAGAAAGATTGCAGAGCTCCAGAT | | | |
| | | AAACAGGGAGGGACTCTCACTCTTTGCTCTAAGTGTGGCAAGGGTTAT | | | |
| | | CATAGAGCTGACCAGTGTCGCTCTGTGAGGGATATAAAGGGCAGAGT | | | |
| | | CCTTCCCCCACCTGATAGTCAATCAGCTGATGTGCCAAAAAACGGGTCA | | | |
| | | TCGGGCCCTCGGTCCCAGGGCCCTCAAAGATATGGGAACCGGTTTGTC | | | |
| | | AGGACCCAGGAAGCAGTCAGAGAGGCGACCCAGGAAGACCCACAAG | | | |
| | | GGTGGACCTGCGTGCCGCCTCCGACTTCCTACTAATGCCTCAAATGAGT | | | |
| | | ATTCAGCCGGTGCCGGTGGAGCCTATACCATCCTTGCCCCCGGGAACC | | | |
| | | ATGGGCCTTATTCTCGGCCGGGGTTCACTCACCTTGCAGGGCTTAGTAG | | | |
| | | TCCACCCTGGAGTTATGGATTGTCAACATTCCCCTGAAATACAGGTCCT | | | |
| | | GTGCTCAAGCCCTAAAGGCGTTTTTTCTATTAGTAAAGGAGATAGGAT | | | |
| | | AGCTCAGCTGCTGCTCCTCCCTGATAATACCAGGGAGAAATTTGCAGG | | | |
| | | ACCTGAGATAAAGAAAATGGGCTCCTCAGGAAATGATTCTGCCTATTT | | | |
| | | GGTTGTATCTTTAAATGATAGACCTAAGCTCCGCCTTAAGATTAACGGA | | | |
| | | AAAGAGTTTGAAGGCATCCTTGATACCGGAGCAGATAAAAGTATAATT | | | |
| | | TCTACACATTGGTGGCCCAAAGCATGGCCCACCACAGAGTCATCTCATT | | | |
| | | CATTACAGGGCCTAGGATATCAATCATGTCCCACTATAAGCTCCATTGC | | | |
| | | CTTGACGTGGGAATCCTCTGAAGGACAGCAAGGGAAATTCATACCTTA | | | |
| | | TGTGCTCCCACTCCCGGTTAACCTCTGGGGAAGGGATATTATGCAGCAT | | | |
| | | TTGGGCCTTATTTTGTCCAATGAAAACGCCCCATCAGGAGGGTATTCAG | | | |
| | | CTAAAGCAAAAAATATCATGGCAAAGATGGGTTATAAAGAAGGAAAA | | | |
| | | GGGTTAGGACATCAAGAACAGGGAAGGATAGAGCCCATCTCACCTAAT | | | |
| | | GGAAACCAAGACAGACAGGGTCTGGGTTTTCCTTAGCGTAATGCTCAA | | | |
| | | GTAgaacaaacgacccaacacccgtgcgttttattctgtctttttattgccgatcccccggccg | | | |
| | | ctttacttgtacagctcgtccatgccgagagtgatcccggcggcggtcacgaactccagcagg | | | |
| | | accatgtgatcgcgcttctcgttggggtctttgctcagggcggactgggtgctcaggtaagtat | | | |
| | | caaggttacaagacaggtttaaggagaccaatagaaactgggcttgtcgagacagagaaga | | | |
| | | ctcttgcgtttctgataggcacctattggtcttactgacatccactttgcctttctctccacag | | | |
| | | gtagtggttgtcgggcagcagcacggggccgtcgccgatgggggtgttctgctggtagtggtcg | | | |
| | | gcgagctgcacgctgccgtcctcgatgttgtggcggatcttgaagttcaccttgatgccgttct | | | |
| | | tctgcttgtcggccatgatatagacgttgtggctgttgtagttgtactccagcttgtgccccag | | | |
| | | gatgttgccgtcctccttgaagtcgatgcccttcagctcgatgcggttcaccagggtgtcgccc | | | |
| | | tcgaacttcacctcggcgcgggtcttgtagttgccgtcgtccttgaagaagatggtgcgctcct | | | |
| | | ggacgtagccttcgggcatggcggacttgaagaagtcgtgctgcttcatgtggtcggggtagcg | | | |
| | | gctgaagcactgcacgccgtaggtcagggtggtcacgagggtgggccagggcacgggcagctt | | | |
| | | gccggtggtgcagatgaacttcagggtcagcttgccgtaggtggcatcgccctcgccctcgcc | | | |
| | | ggacacgctgaacttgtggccgtttacgtcgccgtccagctcgaccaggatgggcaccacccc | | | |
| | | ggtgaacagctcctcgcccttgctcaccatggtggctttaccaacagtaccggaatgccaagc | | | |
| | | ttgggtcctgtgttctggcggcaaacccgttgcgaaaaagaacgttcacggcgactactgcac | | | |
| | | ttatatacggttctcccccaccctcgggaaaaaggcggagccagtacacgacatcactttccc | | | |
| | | agtttaccccgcgccaccttctctaggcaccggatcaattgccgacccctccccccaacttctc | | | |
| | | ggggactgtgggcgatgtgcgctctgcccTTCTCCTGCTTTTTTACCACTAACTAG | | | |
| | | GAACTGGGTTTGGCCTTAATTCAGACAGCCTTGGCTCTGTCTGGACAG | | | |
| | | GTCCAGACAACTGACACCATTAACACTTTGTCAGCCTCAGTGACTACAG | | | |
| | | TCATAGATGAACAGGCCTCAGCTAATGTCAAGATACAGAGAGGTCTCA | | | |
| | | TGCTGGTTAATCAACTCATAGATCTTGTCCAGATACAACTAGATGTATT | | | |
| | | ATGACAATTAACTCAGCTGGGATGTGAACAAAAGTTTCCGGGATTGTG | | | |
| | | TGTTATTTCCATTCAGTATGTTAAATTTACTAGGACAGCTAATTTGTCAA | | | |
| | | AAAGTCTTTTTCAGTATATGTTACAGAATTGGATGGCTGAATTTGAACA | | | |
| | | GATCCTTCGGGAATTGAGACTTCAGGTCAACTCCACGCGCTTGGACCT | | | |
| | | GTCGCTGACCAAAGGATTACCCAATTGGATCTCCTCAGCATTTTCCTTCT | | | |
| | | TTAAAAAATGGGTGGGATTAATATTATTTGGAGATACACTTTGCTGTGG | | | |
| | | ATTAGTGTTGCTTCTTTGATTGGTCTGTAAGCTTAAGGCCTAAACTAGG | | | |
| | | AGAGACAAGGTGGTTATTGCCCAGGCGCTTGCAGGACTAGAACATGG | | | |
| | | AGCTCCCCCTGATATATGGTTATCTATGCTTAGGCAATAGGTCGCTGGC | | | |
| | | CACTCAGCTCTTACATCTCACGAGGCTAGACTCATTGCACGGGATGGA | | | |
| | | GTGAGTGTGCTTCAGCAGCCCGAGAGAGTTGCACGGCTAAGCACTGCA | | | |
| | | ATGGAAAGGCTCTGCGGCATATATGAGCCTATTCTAGGGAGACATGTC | | | |
| | | ATCTTTCATGAAGGTTCAGTGTCCTAGTTCCCTTCCCCCAGGCAAAACG | | | |
| | | ACACGGGAGCAGGTCAGGGTTGCTCTGGGTAAAAGCCTGTGAGCCTA | | | |
| | | AGAGCTAATCCTGTACATGGCTCCTTTACCTACACACTGGGGATTTGAC | | | |
| | | CTCTATCTCCACTCTCATTAATATGGGTGGCCTATTTGCTCTTATTAAAA | | | |
| | | GAAAAGGGGGAGATGTTGGGAGCCGCGCCCACATTCGCCGTTACAAG | | | |
| | | ATGGCGCTGACAGCTGTGTTCTAAGTGGTAAACAAATAATCTGCGCAT | | | |
| | | GTGCCGAGGGTGGTTCTTCACTCTATGTGCTCTGCCTTCCCCGTGACGT | | | |
| | | CAACTCGGCCGATGGGCTGCAGCCAATCAGGGAGTGACACGTCCTAG | | | |
| | | GCGAAGGAGAATTCTCTTTAATAGGGACGGGGTTTTGTTTTCTCTCTCT | | | |
| | | CTTGCTTCTCGCTCGCTCTTGCTTCTTGCACTCTGGCTCCTGAAGATGTA | | | |
| | | AGCAATAAAGTTTTGCCGCAGAAGATTCTGGTCTGTGGTGTTCTTCCTG | | | |
| | | GCCGGGCGTGAGAACGCGTCTAATAACAGCGGCCGCAACAGGGGATC | | | |
| | | CAGACATGATAAGATACATTGATGAGTTTGGACAAACCACAACTAGAA | | | |
| | | TGCAGTGAAAAAAATGCTTTATTTGTGAAATTTGTGATGCTATTGCTTT | | | |
| | | ATTTGTAACCATTATAAGCTGCAATAAACAAGTTAACAACAACAATTGC | | | |
| | | ATTCATTTTATGTTTCAGGTTCAGGGGGAGGTGTGGGAGGTTTTTTCG | | | |
| | | GATCCTCTAGAGTCGACCTGCAGGCATGCAAGCTTGGCGTAATCATGG | | | |
| | | TCATAGCTGTTTCCTGTGTGAAATTGTTATCCGCTCACAATTCCACACAA | | | |
| | | CATACGAGCCGGAAGCATAAAGTGTAAAGCCTGGGGTGCCTAATGAG | | | |
| | | TGAGCTAACTCACATTAATTGCGTTGCGCTCACTGCCCGCTTTCCAGTC | | | |
| | | GGGAAACCTGTCGTGCCAGCTGCATTAATGAATCGGCCAACGCGCGG | | | |
| | | GGAGAGGCGGTTTGCGTATTGGGCGCTCTTCCGCTTCCTCGCTCACTGA | | | |
| | | CTCGCTGCGCTCGGTCGTTCGGCTGCGGCGAGCGGTATCAGCTCACTC | | | |
| | | AAAGGCGGTAATACGGTTATCCACAGAATCAGGGGATAACGCAGGAA | | | |
| | | AGAACATGTGAGCAAAAGGCCAGCAAAAGGCCAGGAACCGTAAAAAG | | | |
| | | GCCGCGTTGCTGGCGTTTTTCCATAGGCTCCGCCCCCCTGACGAGCATC | | | |
| | | ACAAAAATCGACGCTCAAGTCAGAGGTGGCGAAACCCGACAGGACTA | | | |
| | | TAAAGATACCAGGCGTTTCCCCCTGGAAGCTCCCTCGTGCGCTCTCCTG | | | |
| | | TTCCGACCCTGCCGCTTACCGGATACCTGTCCGCCTTTCTCCCTTCGGGA | | | |
| | | AGCGTGGCGCTTTCTCATAGCTCACGCTGTAGGTATCTCAGTTCGGTGT | | | |
| | | AGGTCGTTCGCTCCAAGCTGGGCTGTGTGCACGAACCCCCCGTTCAGC | | | |
| | | CCGACCGCTGCGCCTTATCCGGTAACTATCGTCTTGAGTCCAACCCGGT | | | |
| | | AAGACACGACTTATCGCCACTGGCAGCAGCCACTGGTAACAGGATTAG | | | |
| | | CAGAGCGAGGTATGTAGGCGGTGCTACAGAGTTCTTGAAGTGGTGGC | | | |
| | | CTAACTACGGCTACACTAGAAGGACAGTATTTGGTATCTGCGCTCTGCT | | | |
| | | GAAGCCAGTTACCTTCGGAAAAAGAGTTGGTAGCTCTTGATCCGGCAA | | | |
| | | ACAAACCACCGCTGGTAGCGGTGGTTTTTTTGTTTGCAAGCAGCAGATT | | | |
| | | ACGCGCAGAAAAAAAGGATCTCAAGAAGATCCTTTGATCTTTTCTACG | | | |
| | | GGGTCTGACGCTCAGTGGAACGAAAACTCACGTTAAGGGATTTTGGTC | | | |
| | | ATGAGATTATCAAAAAGGATCTTCACCTAGATCCTTTTAAATTAAAAAT | | | |
| | | GAAGTTTTAAATCAATCTAAAGTATATATGAGTAAACTTGGTCTGACAG | | | |
| | | TTACCAATGCTTAATCAGTGAGGCACCTATCTCAGCGATCTGTCTATTTC | | | |
| | | GTTCATCCATAGTTGCCTGACTCCCCGTCGTGTAGATAACTACGATACG | | | |
| | | GGAGGGCTTACCATCTGGCCCCAGTGCTGCAATGATACCGCGAGACCC | | | |
| | | ACGCTCACCGGCTCCAGATTTATCAGCAATAAACCAGCCAGCCGGAAG | | | |
| | | GGCCGAGCGCAGAAGTGGTCCTGCAACTTTATCCGCCTCCATCCAGTCT | | | |
| | | ATTAATTGTTGCCGGGAAGCTAGAGTAAGTAGTTCGCCAGTTAATAGT | | | |
| | | TTGCGCAACGTTGTTGCCATTGCTACAGGCATCGTGGTGTCACGCTCGT | | | |
| | | CGTTTGGTATGGCTTCATTCAGCTCCGGTTCCCAACGATCAAGGCGAGT | | | |
| | | TACATGATCCCCCATGTTGTGCAAAAAAGCGGTTAGCTCCTTCGGTCCT | | | |
| | | CCGATCGTTGTCAGAAGTAAGTTGGCCGCAGTGTTATCACTCATGGTTA | | | |
| | | TGGCAGCACTGCATAATTCTCTTACTGTCATGCCATCCGTAAGATGCTTT | | | |
| | | TCTGTGACTGGTGAGTACTCAACCAAGTCATTCTGAGAATAGTGTATGC | | | |
| | | GGCGACCGAGTTGCTCTTGCCCGGCGTCAATACGGGATAATACCGCGC | | | |
| | | CACATAGCAGAACTTTAAAAGTGCTCATCATTGGAAAACGTTCTTCGGG | | | |
| | | GCGAAAACTCTCAAGGATCTTACCGCTGTTGAGATCCAGTTCGATGTAA | | | |
| | | CCCACTCGTGCACCCAACTGATCTTCAGCATCTTTTACTTTCACCAGCGT | | | |
| | | TTCTGGGTGAGCAAAAACAGGAAGGCAAAATGCCGCAAAAAAGGGAA | | | |
| | | TAAGGGCGACACGGAAATGTTGAATACTCATACTCTTCCTTTTTCAATA | | | |
| | | TTATTGAAGCATTTATCAGGGTTATTGTCTCATGAGCGGATACATATTT | | | |
| | | GAATGTATTTAGAAAAATAAACAAATAGGGGTTCCGCGCACATTTCCCC | | | |
| | | GAAAAGTGCCACCTGACGTCTAAGAAACCATTATTATCATGACATTAAC | | | |
| | | CTATAAAAATAGGCGTATCACGAGGCCCTTTCGTCTCGCGCGTTTCGGT | | | |
| | | GATGACGGTGAAAACCTCTGACACATGCAGCTCCCGGAGACGGTCACA | | | |
| | | GCTTGTCTGTAAGCGGATGCCGGGAGCAGACAAGCCCGTCAGGGCGC | | | |
| | | GTCAGCGGGTGTTGGCGGGTGTCGGGGCTGGCTTAACTATGCGGCAT | | | |
| | | CAGAGCAGATTGTACTGAGAGTGCACCATATGCGGTGTGAAATACCGC | | | |
| | | ACAGATGCGTAAGGAGAAAATACCGCATCAGGCGCCATTCGCCATTCA | | | |
| | | GGCTGCGCAACTGTTGGGAAGGGCGATCGGTGCGGGCCTCTTCGCTAT | | | |
| | | TACGCCAGCTGGCGAAAGGGGGATGTGCTGCAAGGCGATTAAGTTGG | | | |
| | | GTAACGCCAGGGTTTTCCCAGTCACGACGTTGTAAAACGACGGCCAGT | | | |
| CIS_pCMV_ | PLV10023 | GAATTCGAGCTTGCATGCCTGCAGGTCGTTACATAACTTACGGTAAATG | 358 | N/A | |
| R-U5- | | GCCCGCCTGGCTGACCGCCCAACGACCCCCGCCCATTGACGTCAATAAT | | | |
| IAP- | | GACGTATGTTCCCATAGTAACGCCAATAGGGACTTTCCATTGACGTCAA | | | |
| 92L23_ | | TGGGTGGAGTATTTACGGTAAACTGCCCACTTGGCAGTACATCAAGTG | | | |
| EF1- | | TATCATATGCCAAGTACGCCCCCTATTGACGTCAATGACGGTAAATGGC | | | |
| GFPai- | | CCGCCTGGCATTATGCCCAGTACATGACCTTATGGGACTTTCCTACTTG | | | |
| LTR_v1 | | GCAGTACATCTACGTATTAGTCATCGCTATTACCATGGTGATGCGGTTT | | | |
| | | TGGCAGTACATCAATGGGCGTGGATAGCGGTTTGACTCACGGGGATTT | | | |
| | | CCAAGTCTCCACCCCATTGACGTCAATGGGAGTTTGTTTTGGCACCAAA | | | |
| | | ATCAACGGGACTTTCCAAAATGTCGTAACAACTCCGCCCCATTGACGCA | | | |
| | | AATGGGCGGTAGGCGTGTACGGTGGGAGGTCTATATAAGCAGAGCTC | | | |
| | | GTTTAGTGAACCGTCAGGGACGGGGTTTCGTTTTCTCTCTCTCTTGCTTC | | | |
| | | TCGCTCGCTCTTGCTTCTTGCACTCTGGCTCCTGAAGATGTAAGCAATA | | | |
| | | AAGTTTTGCCGCAGAAGATTCTGGTCTGTGGTGTTCTTCCTGGCCGGGC | | | |
| | | GTGAGAACGCGTCTAATAACAATTGGTGCCGAATTCCGGGACGAGAAA | | | |
| | | AAAACTCGGGACTGGCGCAAGGAAGATCCCTCATTCCAGAACCAGAAC | | | |
| | | TGCGGGTCGCGGTAATAAAGGTTCCCGTAAAGCAGACTGTTAAGAAG | | | |
| | | GATTCAACTGTATGAATTCAGAACTTTTCAGCTGGGGAACGAGAGTAC | | | |
| | | CAGTGAGTACAGCTTTACGAGGTAAGTCTGATCTTGAACTTTCTAAGGA | | | |
| | | AATTCAAGACAGTCTATCAGAAGTAAAGTGGAATATGTTTGGCCTTGA | | | |
| | | ATTTTTTCTGGTGTTAGGAGCCCTTTTGTTCCTTTTCACATGTTATCAAG | | | |
| | | TGATTAAGATAGGGCTGAAAATTCTAGAGGAAATTCAGGACAAGCTAT | | | |
| | | CAGAAGTAAAGCGGGGAGAGAGAGTAGGAGCAAAGAGAAAATATGG | | | |
| | | TACACAAAATAAGTATACAGGCCTTTCCAAGGGTCTTGAACCCGAGGA | | | |
| | | AAAGTTAAGGTTAGGTAGGAATACCTGGAGAGAGATTAGAAGAAAAA | | | |
| | | GAGGAAAAAGGGAAAAGAAGAAAGATCAATTAGCGGAGGTCTCTAG | | | |
| | | GAGATACTCGTCACTAGATGAGCTCAGGAAGCCAGCTCTTAGTAGTTC | | | |
| | | TGAAGCAGATGAAGAATTCTCCTCTGAGGAAACAGACTGGGAGGAAG | | | |
| | | AAGCAGCCCATTACCAGCCAGCTAATTGGTCAAGAAAAAAGCCAAAAG | | | |
| | | CGGCTGGCGAAGGCCAGTTTGCTGATTGGCCTCAGGGCAGTCGGCTTC | | | |
| | | AAGGTCCGCCCTATGCGGAGTCCCCGCCCTGCGTAGTGCGTCAGCAAT | | | |
| | | GCGCAGAGAGGCAGTGCGCAGAGAGGCAGTGCGCAGACTCATTCATT | | | |
| | | CCCAGAGAGGAACAAAGGAAAATACAACAGGCATTTCCGGTCTTTGAA | | | |
| | | GGAGCCGAGGGTGGGCGTGTCCACGCTCCGGTAGAATACTTACAAATT | | | |
| | | AAAGAAATTGCCGAGTCGGTCCGTAAATATGGAACCAATGCTAATTTT | | | |
| | | ACCTTGGTGCAGTTAGACAGGCTCGCCGGCATGGCACTAACTCCTGCT | | | |
| | | GACTGGCAAACGGTTGTAAAAGCCGCTCTCCCTAGTATGGGCAAATAT | | | |
| | | ATGGAATGGAGAGCGCTTTGGCACGAAGCTGCACAAGCGCAGGCCCG | | | |
| | | AGCAAACGCAGCTGCTTTGACTCCAGAGCAGAGAGATTGGACTTTTGA | | | |
| | | CTTGTTAACGGGTCAGGGAGCTTATTCTGCTGATCAGACAAACTACCAT | | | |
| | | TGGGGAGCTTATGCCCAGATTTCTTCCACGGCTATTAGGGCCTGGAAG | | | |
| | | GCGCTCTCTCGAGCAGGTGAAACCACTGGTCAGTTAACAAAGATAATC | | | |
| | | CAGGGACCTCAGGAATCCTTCTCAGATTTTGTGGCCAGAATGACAGAG | | | |
| | | GCAGCAGAGCGTATTTTTGGAGAGTCAGAGCAAGCTGCGCCTCTGATA | | | |
| | | GAACAGCTAATCTATGAGCAAGCCACAAAGGAGTGCCGAGCGGCCAT | | | |
| | | AGCCCCAAGAAAGAACAAAGGCTTACAAGACTGGCTCAGGGTCTGTCG | | | |
| | | AGAGCTTGGGGGACCTCTCAGCAATGCAGGTTTAGCGGCTGCCATCCT | | | |
| | | TCAATCCCAAAACCGCTCCATGGGCAGAAATAATCAGAGGACATGTTTT | | | |
| | | AACTGCGGAAAGCCTGGGCATTTTAAGAAAGATTGCAGAGCTCCAGAT | | | |
| | | AAACAGGGAGGGACTCTCACTCTTTGCTCTAAGTGTGGCAAGGGTTAT | | | |
| | | CATAGAGCTGACCAGTGTCGCTCTGTGAGGGATATAAAGGGCAGAGT | | | |
| | | CCTTCCCCCACCTGATAGTCAATCAGCTGATGTGCCAAAAAACGGGTCA | | | |
| | | TCGGGCCCTCGGTCCCAGGGCCCTCAAAGATATGGGAACCGGTTTGTC | | | |
| | | AGGACCCAGGAAGCAGTCAGAGAGGCGACCCAGGAAGACCCACAAG | | | |
| | | GGTGGACCTGCGTGCCGCCTCCGACTTCCTACTAATGCCTCAAATGAGT | | | |
| | | ATTCAGCCGGTGCCGGTGGAGCCTATACCATCCTTGCCCCCGGGAACC | | | |
| | | ATGGGCCTTATTCTCGGCCGGGGTTCACTCACCTTGCAGGGCTTAGTAG | | | |
| | | TCCACCCTGGAGTTATGGATTGTCAACATTCCCCTGAAATACAGGTCCT | | | |
| | | GTGCTCAAGCCCTAAAGGCGTTTTTTCTATTAGTAAAGGAGATAGGAT | | | |
| | | AGCTCAGCTGCTGCTCCTCCCTGATAATACCAGGGAGAAATTTGCAGG | | | |
| | | ACCTGAGATAAAGAAAATGGGCTCCTCAGGAAATGATTCTGCCTATTT | | | |
| | | GGTTGTATCTTTAAATGATAGACCTAAGCTCCGCCTTAAGATTAACGGA | | | |
| | | AAAGAGTTTGAAGGCATCCTTGATACCGGAGCAGATAAAAGTATAATT | | | |
| | | TCTACACATTGGTGGCCCAAAGCATGGCCCACCACAGAGTCATCTCATT | | | |
| | | CATTACAGGGCCTAGGATATCAATCATGTCCCACTATAAGCTCCATTGC | | | |
| | | CTTGACGTGGGAATCCTCTGAAGGACAGCAAGGGAAATTCATACCTTA | | | |
| | | TGTGCTCCCACTCCCGGTTAACCTCTGGGGAAGGGATATTATGCAGCAT | | | |
| | | TTGGGCCTTATTTTGTCCAATGAAAACGCCCCATCAGGAGGGTATTCAG | | | |
| | | CTAAAGCAAAAAATATCATGGCAAAGATGGGTTATAAAGAAGGAAAA | | | |
| | | GGGTTAGGACATCAAGAACAGGGAAGGATAGAGCCCATCTCACCTAAT | | | |
| | | GGAAACCAAGACAGACAGGGTCTGGGTTTTCCTTAGCGGCCATTGGG | | | |
| | | GCAGCACGACCCATACCATGGAAAACAGGGGACCCAGTGTGGGTTCCT | | | |
| | | CAATGGCACCTATCCTCTGAAAAACTAGAAGCTGTGATTCAACTGGTA | | | |
| | | GAGGAACAATTAAAACTAGGCCATATTGAACCCTCTACCTCACCTTGGA | | | |
| | | ATACTCCAATTTTTGTAATTAAGAAAAAGTCAGGAAAGTGGAGACTGC | | | |
| | | TCCATGACCTCAGAGCCATTAATGAGCAAATGAACTTATTTGGCCCAGT | | | |
| | | ACAGAGGGGTCTCCCTGTACTTTCCGCCTTACCACGTGGCTGGAATTTA | | | |
| | | ATTATTATAGATATTAAAGATTGTTTCTTTTCTATACCTTTGTGTCCAAG | | | |
| | | GGATAGGCCCAGATTTGCCTTTACCATCCCCTCTATTAATCACATGGAA | | | |
| | | CCTGATAAGAGGTATCAATGGAAGGTCTTACCACAGGGAATGTCCAAT | | | |
| | | AGTCCTACAATGTGCCAACTTTATGTGCAAGAAGCTCTTTTGCCAGTGA | | | |
| | | GGGAACAATTCCCCTCTTTAATTTTGCTCCTTTACATGGATGACATCCTC | | | |
| | | CTGTGCCATAAAGACCTTACCATGCTACAAAAGGCATATCCTTTTCTACT | | | |
| | | TAAAACTTTAAGTCAGTGGGGTTTACAGATAGCCACAGAAAAGGTCCA | | | |
| | | AATTTCTGATACAGGACAATTCTTGGGCTCTGTGGTGTCCCCAGATAAG | | | |
| | | ATTGTGCCCCAAAAGGTAGAGATAAGAAGAGATCACCTCCATACCTTA | | | |
| | | AATGATTTTCAAAAGCTGTTGGGAGATATTAATTGGCTCAGACCTTTTT | | | |
| | | TAAAGATTCCTTCCGCTGAGTTAAGGCCTTTGTTTAGTATTTTAGAAGG | | | |
| | | AGATCCTCATATCTCCTCCCCTAGGACTCTTACTCTAGCTGCTAACCAGG | | | |
| | | CCTTACAAAAGGTGGAAAAAGCCTTACAGAATGCACAATTACAACGTA | | | |
| | | TTGAGGATTCGCAGCCTTTCAGTTTGTGTGTCTTTAAGACAGCACAATT | | | |
| | | GCCAACTGCAGTTTTGTGGCAGAATGGGCCATTGTTGTGGATCCATCC | | | |
| | | AAACGTATCCCCAGCTAAAATAATAGATTGGTATCCTGATGCAATTGCA | | | |
| | | CAGCTTGCCCTTAAAGGTCTAAAAGCAGCAATCACCCACTTTGGGCAAA | | | |
| | | GTCCATATCTTTTAATTGTACCTTATACCGCTGCACAGGTTCAAACCTTG | | | |
| | | GCAGCCACATCTAATGATTGGGCAGTTTTAGTTACCTCCTTTTCAGGAA | | | |
| | | AAATAGATAACCATTATCCAAAACATCCAATCTTACAGTTTGCCCAAAA | | | |
| | | TCAATCTGTTGTGTTTCCACAAATAACAGTAAGAAACCCACTTAAAAAT | | | |
| | | GGGATTGTGGTATATACTGATGGATCAAAAACTGGCATAGGTGCCTAT | | | |
| | | GTGGCTAATGGTAAAGTGGTATCCAAACAATATAATGAAAATTCACCTC | | | |
| | | AAGTGGTAGAATGTTTAGTGGTCTTAGAAGTTTTAAAAACCTTTTTAGA | | | |
| | | ACCCCTTAATATTGTGTCAGATTCCTGTTATGTGGTTAATGCAGTAAATC | | | |
| | | TTTTAGAAGTGGCTGGAGTGATTAAGCCTTCCAGTAGAGTTGCCAATAT | | | |
| | | TTTTCAGCAGATACAATTAGTTTTGTTATCTAGAAGATTTCCTGTTTATA | | | |
| | | TTACTCATGTTAGAGCCCATTCAGGCCTACCTGGCCCCATGGCTCTGGG | | | |
| | | AAATAATTTGGCAGATAAGGCCACTAAAGTGGTGGCTGCTGCCCTATC | | | |
| | | ATCCCCGGTAGAGGCTGCAAGAAATTTTCATAACAATTTTCATGTGACG | | | |
| | | GCTGAAACATTACGCAGTCGTTTCTCCTTGACAAGAAAAGAAGCCCGT | | | |
| | | GACATTGTTACTCAATGTCAAAGCTGCTGTGAGTTCTTGCCAGTTCCTC | | | |
| | | ATGTGGGAATTAACCCACGCGGTATTCGACCTCTACAGGTCTGGCAAA | | | |
| | | TGGATGTTACACATGTTTCTTCCTTTGGAAAACTTCAATATCTCCATGTG | | | |
| | | TCCATTGACACATGTTCTGGCATCATGTTTGCTTCTCCATTAACCGGAGA | | | |
| | | AAAAGCCTCACATGTGATTCAACATTGCCTTGAGGCATGGAGTGCTTG | | | |
| | | GGGGAAACCCAGACTCCTTAAGACTGATAATGGACCAGCTTATACGTC | | | |
| | | TCAAAAATTCCAACAGTTCTGCCGTCAGATGGACGTGACCCACCTGACT | | | |
| | | GGACTTCCATACAACCCTCAAGGACAGGGTATTGTTGAGCGTGCGCAT | | | |
| | | CGCACCCTCAAAACCTATCTTATAAAACAGAAGAGGGGAACTTTTGAG | | | |
| | | GAGACTGTACCCCGAGCACCAAGAGTGTCGGTGTCTATGGCACTCTTT | | | |
| | | ACACTCAATTTTTTAAATATTGATGCTCATGGCCATACTGCGGCTGAAC | | | |
| | | GTCATTGTACAGAGCCAGATAGGCCCAATGAGATGGTTAAATGGAAAA | | | |
| | | ATGTCCTTGATAATAAATGGTATGGCCCGGATCCTATTTTGATAAGATC | | | |
| | | CAGGGGAGCTATCTGTGTTTTCCCACAGAATGAAAACAACCCATTTTGG | | | |
| | | ATACCAGAAAGACTCACCCGAAAAATCCAGACTGACCAAGGAAATACT | | | |
| | | AATGTCCCTCGTCTTGGTGATGTCCAGGGCGTCAATAATAAAAAGAGA | | | |
| | | GCAGCGTTGGGGGATAATGTCGACATTTCCACTCCCAATGACGGTGAT | | | |
| | | GTATAATGCTCAAGTAgaacaaacgacccaacacccgtgcgttttattctgtctttttat | | | |
| | | tgccgatcccccggccgctttacttgtacagctcgtccatgccgagagtgatcccggcggcggt | | | |
| | | cacgaactccagcaggaccatgtgatcgcgcttctcgttggggtctttgctcagggcggactg | | | |
| | | ggtgctcaggtaagtatcaaggttacaagacaggtttaaggagaccaatagaaactgggctt | | | |
| | | gtcgagacagagaagactcttgcgtttctgataggcacctattggtcttactgacatccacttt | | | |
| | | gcctttctctccacaggtagtggttgtcgggcagcagcacggggccgtcgccgatgggggtgt | | | |
| | | tctgctggtagtggtcggcgagctgcacgctgccgtcctcgatgttgtggcggatcttgaagtt | | | |
| | | caccttgatgccgttcttctgcttgtcggccatgatatagacgttgtggctgttgtagttgtac | | | |
| | | tccagcttgtgccccaggatgttgccgtcctccttgaagtcgatgcccttcagctcgatgcggt | | | |
| | | tcaccagggtgtcgccctcgaacttcacctcggcgcgggtcttgtagttgccgtcgtccttgaa | | | |
| | | gaagatggtgcgctcctggacgtagccttcgggcatggcggacttgaagaagtcgtgctgcttc | | | |
| | | atgtggtcggggtagcggctgaagcactgcacgccgtaggtcagggtggtcacgagggtgggcc | | | |
| | | agggcacgggcagcttgccggtggtgcagatgaacttcagggtcagcttgccgtaggtggca | | | |
| | | tcgccctcgccctcgccggacacgctgaacttgtggccgtttacgtcgccgtccagctcgacca | | | |
| | | ggatgggcaccaccccggtgaacagctcctcgcccttgctcaccatggtggctttaccaacag | | | |
| | | taccggaatgccaagcttgggtcctgtgttctggcggcaaacccgttgcgaaaaagaacgttc | | | |
| | | acggcgactactgcacttatatacggttctcccccaccctcgggaaaaaggcggagccagta | | | |
| | | cacgacatcactttcccagtttaccccgcgccaccttctctaggcaccggatcaattgccgacc | | | |
| | | cctccccccaacttctcggggactgtgggcgatgtgcgctctgcccTTCTCCTGCTTTTTT | | | |
| | | ACCACTAACTAGGAACTGGGTTTGGCCTTAATTCAGACAGCCTTGGCTC | | | |
| | | TGTCTGGACAGGTCCAGACAACTGACACCATTAACACTTTGTCAGCCTC | | | |
| | | AGTGACTACAGTCATAGATGAACAGGCCTCAGCTAATGTCAAGATACA | | | |
| | | GAGAGGTCTCATGCTGGTTAATCAACTCATAGATCTTGTCCAGATACAA | | | |
| | | CTAGATGTATTATGACAATTAACTCAGCTGGGATGTGAACAAAAGTTTC | | | |
| | | CGGGATTGTGTGTTATTTCCATTCAGTATGTTAAATTTACTAGGACAGC | | | |
| | | TAATTTGTCAAAAAGTCTTTTTCAGTATATGTTACAGAATTGGATGGCT | | | |
| | | GAATTTGAACAGATCCTTCGGGAATTGAGACTTCAGGTCAACTCCACG | | | |
| | | CGCTTGGACCTGTCGCTGACCAAAGGATTACCCAATTGGATCTCCTCAG | | | |
| | | CATTTTCCTTCTTTAAAAAATGGGTGGGATTAATATTATTTGGAGATAC | | | |
| | | ACTTTGCTGTGGATTAGTGTTGCTTCTTTGATTGGTCTGTAAGCTTAAG | | | |
| | | GCCTAAACTAGGAGAGACAAGGTGGTTATTGCCCAGGCGCTTGCAGG | | | |
| | | ACTAGAACATGGAGCTCCCCCTGATATATGGTTATCTATGCTTAGGCAA | | | |
| | | TAGGTCGCTGGCCACTCAGCTCTTACATCTCACGAGGCTAGACTCATTG | | | |
| | | CACGGGATGGAGTGAGTGTGCTTCAGCAGCCCGAGAGAGTTGCACGG | | | |
| | | CTAAGCACTGCAATGGAAAGGCTCTGCGGCATATATGAGCCTATTCTA | | | |
| | | GGGAGACATGTCATCTTTCATGAAGGTTCAGTGTCCTAGTTCCCTTCCC | | | |
| | | CCAGGCAAAACGACACGGGAGCAGGTCAGGGTTGCTCTGGGTAAAAG | | | |
| | | CCTGTGAGCCTAAGAGCTAATCCTGTACATGGCTCCTTTACCTACACAC | | | |
| | | TGGGGATTTGACCTCTATCTCCACTCTCATTAATATGGGTGGCCTATTTG | | | |
| | | CTCTTATTAAAAGAAAAGGGGGAGATGTTGGGAGCCGCGCCCACATTC | | | |
| | | GCCGTTACAAGATGGCGCTGACAGCTGTGTTCTAAGTGGTAAACAAAT | | | |
| | | AATCTGCGCATGTGCCGAGGGTGGTTCTTCACTCTATGTGCTCTGCCTT | | | |
| | | CCCCGTGACGTCAACTCGGCCGATGGGCTGCAGCCAATCAGGGAGTG | | | |
| | | ACACGTCCTAGGCGAAGGAGAATTCTCTTTAATAGGGACGGGGTTTTG | | | |
| | | TTTTCTCTCTCTCTTGCTTCTCGCTCGCTCTTGCTTCTTGCACTCTGGCTC | | | |
| | | CTGAAGATGTAAGCAATAAAGTTTTGCCGCAGAAGATTCTGGTCTGTG | | | |
| | | GTGTTCTTCCTGGCCGGGCGTGAGAACGCGTCTAATAACAGCGGCCGC | | | |
| | | AACAGGGGATCCAGACATGATAAGATACATTGATGAGTTTGGACAAAC | | | |
| | | CACAACTAGAATGCAGTGAAAAAAATGCTTTATTTGTGAAATTTGTGAT | | | |
| | | GCTATTGCTTTATTTGTAACCATTATAAGCTGCAATAAACAAGTTAACA | | | |
| | | ACAACAATTGCATTCATTTTATGTTTCAGGTTCAGGGGGAGGTGTGGG | | | |
| | | AGGTTTTTTCGGATCCTCTAGAGTCGACCTGCAGGCATGCAAGCTTGGC | | | |
| | | GTAATCATGGTCATAGCTGTTTCCTGTGTGAAATTGTTATCCGCTCACA | | | |
| | | ATTCCACACAACATACGAGCCGGAAGCATAAAGTGTAAAGCCTGGGGT | | | |
| | | GCCTAATGAGTGAGCTAACTCACATTAATTGCGTTGCGCTCACTGCCCG | | | |
| | | CTTTCCAGTCGGGAAACCTGTCGTGCCAGCTGCATTAATGAATCGGCCA | | | |
| | | ACGCGCGGGGAGAGGCGGTTTGCGTATTGGGCGCTCTTCCGCTTCCTC | | | |
| | | GCTCACTGACTCGCTGCGCTCGGTCGTTCGGCTGCGGCGAGCGGTATC | | | |
| | | AGCTCACTCAAAGGCGGTAATACGGTTATCCACAGAATCAGGGGATAA | | | |
| | | CGCAGGAAAGAACATGTGAGCAAAAGGCCAGCAAAAGGCCAGGAACC | | | |
| | | GTAAAAAGGCCGCGTTGCTGGCGTTTTTCCATAGGCTCCGCCCCCCTGA | | | |
| | | CGAGCATCACAAAAATCGACGCTCAAGTCAGAGGTGGCGAAACCCGA | | | |
| | | CAGGACTATAAAGATACCAGGCGTTTCCCCCTGGAAGCTCCCTCGTGC | | | |
| | | GCTCTCCTGTTCCGACCCTGCCGCTTACCGGATACCTGTCCGCCTTTCTC | | | |
| | | CCTTCGGGAAGCGTGGCGCTTTCTCATAGCTCACGCTGTAGGTATCTCA | | | |
| | | GTTCGGTGTAGGTCGTTCGCTCCAAGCTGGGCTGTGTGCACGAACCCC | | | |
| | | CCGTTCAGCCCGACCGCTGCGCCTTATCCGGTAACTATCGTCTTGAGTC | | | |
| | | CAACCCGGTAAGACACGACTTATCGCCACTGGCAGCAGCCACTGGTAA | | | |
| | | CAGGATTAGCAGAGCGAGGTATGTAGGCGGTGCTACAGAGTTCTTGA | | | |
| | | AGTGGTGGCCTAACTACGGCTACACTAGAAGGACAGTATTTGGTATCT | | | |
| | | GCGCTCTGCTGAAGCCAGTTACCTTCGGAAAAAGAGTTGGTAGCTCTT | | | |
| | | GATCCGGCAAACAAACCACCGCTGGTAGCGGTGGTTTTTTTGTTTGCAA | | | |
| | | GCAGCAGATTACGCGCAGAAAAAAAGGATCTCAAGAAGATCCTTTGAT | | | |
| | | CTTTTCTACGGGGTCTGACGCTCAGTGGAACGAAAACTCACGTTAAGG | | | |
| | | GATTTTGGTCATGAGATTATCAAAAAGGATCTTCACCTAGATCCTTTTA | | | |
| | | AATTAAAAATGAAGTTTTAAATCAATCTAAAGTATATATGAGTAAACTT | | | |
| | | GGTCTGACAGTTACCAATGCTTAATCAGTGAGGCACCTATCTCAGCGAT | | | |
| | | CTGTCTATTTCGTTCATCCATAGTTGCCTGACTCCCCGTCGTGTAGATAA | | | |
| | | CTACGATACGGGAGGGCTTACCATCTGGCCCCAGTGCTGCAATGATAC | | | |
| | | CGCGAGACCCACGCTCACCGGCTCCAGATTTATCAGCAATAAACCAGC | | | |
| | | CAGCCGGAAGGGCCGAGCGCAGAAGTGGTCCTGCAACTTTATCCGCCT | | | |
| | | CCATCCAGTCTATTAATTGTTGCCGGGAAGCTAGAGTAAGTAGTTCGCC | | | |
| | | AGTTAATAGTTTGCGCAACGTTGTTGCCATTGCTACAGGCATCGTGGTG | | | |
| | | TCACGCTCGTCGTTTGGTATGGCTTCATTCAGCTCCGGTTCCCAACGAT | | | |
| | | CAAGGCGAGTTACATGATCCCCCATGTTGTGCAAAAAAGCGGTTAGCT | | | |
| | | CCTTCGGTCCTCCGATCGTTGTCAGAAGTAAGTTGGCCGCAGTGTTATC | | | |
| | | ACTCATGGTTATGGCAGCACTGCATAATTCTCTTACTGTCATGCCATCC | | | |
| | | GTAAGATGCTTTTCTGTGACTGGTGAGTACTCAACCAAGTCATTCTGAG | | | |
| | | AATAGTGTATGCGGCGACCGAGTTGCTCTTGCCCGGCGTCAATACGGG | | | |
| | | ATAATACCGCGCCACATAGCAGAACTTTAAAAGTGCTCATCATTGGAAA | | | |
| | | ACGTTCTTCGGGGCGAAAACTCTCAAGGATCTTACCGCTGTTGAGATCC | | | |
| | | AGTTCGATGTAACCCACTCGTGCACCCAACTGATCTTCAGCATCTTTTAC | | | |
| | | TTTCACCAGCGTTTCTGGGTGAGCAAAAACAGGAAGGCAAAATGCCGC | | | |
| | | AAAAAAGGGAATAAGGGCGACACGGAAATGTTGAATACTCATACTCTT | | | |
| | | CCTTTTTCAATATTATTGAAGCATTTATCAGGGTTATTGTCTCATGAGCG | | | |
| | | GATACATATTTGAATGTATTTAGAAAAATAAACAAATAGGGGTTCCGC | | | |
| | | GCACATTTCCCCGAAAAGTGCCACCTGACGTCTAAGAAACCATTATTAT | | | |
| | | CATGACATTAACCTATAAAAATAGGCGTATCACGAGGCCCTTTCGTCTC | | | |
| | | GCGCGTTTCGGTGATGACGGTGAAAACCTCTGACACATGCAGCTCCCG | | | |
| | | GAGACGGTCACAGCTTGTCTGTAAGCGGATGCCGGGAGCAGACAAGC | | | |
| | | CCGTCAGGGCGCGTCAGCGGGTGTTGGCGGGTGTCGGGGCTGGCTTA | | | |
| | | ACTATGCGGCATCAGAGCAGATTGTACTGAGAGTGCACCATATGCGGT | | | |
| | | GTGAAATACCGCACAGATGCGTAAGGAGAAAATACCGCATCAGGCGC | | | |
| | | CATTCGCCATTCAGGCTGCGCAACTGTTGGGAAGGGCGATCGGTGCGG | | | |
| | | GCCTCTTCGCTATTACGCCAGCTGGCGAAAGGGGGATGTGCTGCAAGG | | | |
| | | CGATTAAGTTGGGTAACGCCAGGGTTTTCCCAGTCACGACGTTGTAAA | | | |
| | | ACGACGGCCAGT | | | |
| CIS_pCMV_ | PLV10031 | GAATTCGAGCTTGCATGCCTGCAGGTCGTTACATAACTTACGGTAAATG | 359 | N/A | |
| R-U5- | | GCCCGCCTGGCTGACCGCCCAACGACCCCCGCCCATTGACGTCAATAAT | | | |
| PBS*- | | GACGTATGTTCCCATAGTAACGCCAATAGGGACTTTCCATTGACGTCAA | | | |
| IAP- | | TGGGTGGAGTATTTACGGTAAACTGCCCACTTGGCAGTACATCAAGTG | | | |
| 92L23_ | | TATCATATGCCAAGTACGCCCCCTATTGACGTCAATGACGGTAAATGGC | | | |
| EF1- | | CCGCCTGGCATTATGCCCAGTACATGACCTTATGGGACTTTCCTACTTG | | | |
| GFPai- | | GCAGTACATCTACGTATTAGTCATCGCTATTACCATGGTGATGCGGTTT | | | |
| LTR_v1 | | TGGCAGTACATCAATGGGCGTGGATAGCGGTTTGACTCACGGGGATTT | | | |
| | | CCAAGTCTCCACCCCATTGACGTCAATGGGAGTTTGTTTTGGCACCAAA | | | |
| | | ATCAACGGGACTTTCCAAAATGTCGTAACAACTCCGCCCCATTGACGCA | | | |
| | | AATGGGCGGTAGGCGTGTACGGTGGGAGGTCTATATAAGCAGAGCTC | | | |
| | | GTTTAGTGAACCGTCAGGGACGGGGTTTCGTTTTCTCTCTCTCTTGCTTC | | | |
| | | TCGCTCGCTCTTGCTTCTTGCACTCTGGCTCCTGAAGATGTAAGCAATA | | | |
| | | AAGTTTTGCCGCAGAAGATTCTGGTCTGTGGTGTTCTTCCTGGCCGGGC | | | |
| | | GTGAGAACGCGTCTAATAACAATTTAGCGACTAAACACATCACGAGAA | | | |
| | | AAAAACTCGGGACTGGCGCAAGGAAGATCCCTCATTCCAGAACCAGAA | | | |
| | | CTGCGGGTCGCGGTAATAAAGGTTCCCGTAAAGCAGACTGTTAAGAAG | | | |
| | | GATTCAACTGTATGAATTCAGAACTTTTCAGCTGGGGAACGAGAGTAC | | | |
| | | CAGTGAGTACAGCTTTACGAGGTAAGTCTGATCTTGAACTTTCTAAGGA | | | |
| | | AATTCAAGACAGTCTATCAGAAGTAAAGTGGAATATGTTTGGCCTTGA | | | |
| | | ATTTTTTCTGGTGTTAGGAGCCCTTTTGTTCCTTTTCACATGTTATCAAG | | | |
| | | TGATTAAGATAGGGCTGAAAATTCTAGAGGAAATTCAGGACAAGCTAT | | | |
| | | CAGAAGTAAAGCGGGGAGAGAGAGTAGGAGCAAAGAGAAAATATGG | | | |
| | | TACACAAAATAAGTATACAGGCCTTTCCAAGGGTCTTGAACCCGAGGA | | | |
| | | AAAGTTAAGGTTAGGTAGGAATACCTGGAGAGAGATTAGAAGAAAAA | | | |
| | | GAGGAAAAAGGGAAAAGAAGAAAGATCAATTAGCGGAGGTCTCTAG | | | |
| | | GAGATACTCGTCACTAGATGAGCTCAGGAAGCCAGCTCTTAGTAGTTC | | | |
| | | TGAAGCAGATGAAGAATTCTCCTCTGAGGAAACAGACTGGGAGGAAG | | | |
| | | AAGCAGCCCATTACCAGCCAGCTAATTGGTCAAGAAAAAAGCCAAAAG | | | |
| | | CGGCTGGCGAAGGCCAGTTTGCTGATTGGCCTCAGGGCAGTCGGCTTC | | | |
| | | AAGGTCCGCCCTATGCGGAGTCCCCGCCCTGCGTAGTGCGTCAGCAAT | | | |
| | | GCGCAGAGAGGCAGTGCGCAGAGAGGCAGTGCGCAGACTCATTCATT | | | |
| | | CCCAGAGAGGAACAAAGGAAAATACAACAGGCATTTCCGGTCTTTGAA | | | |
| | | GGAGCCGAGGGTGGGCGTGTCCACGCTCCGGTAGAATACTTACAAATT | | | |
| | | AAAGAAATTGCCGAGTCGGTCCGTAAATATGGAACCAATGCTAATTTT | | | |
| | | ACCTTGGTGCAGTTAGACAGGCTCGCCGGCATGGCACTAACTCCTGCT | | | |
| | | GACTGGCAAACGGTTGTAAAAGCCGCTCTCCCTAGTATGGGCAAATAT | | | |
| | | ATGGAATGGAGAGCGCTTTGGCACGAAGCTGCACAAGCGCAGGCCCG | | | |
| | | AGCAAACGCAGCTGCTTTGACTCCAGAGCAGAGAGATTGGACTTTTGA | | | |
| | | CTTGTTAACGGGTCAGGGAGCTTATTCTGCTGATCAGACAAACTACCAT | | | |
| | | TGGGGAGCTTATGCCCAGATTTCTTCCACGGCTATTAGGGCCTGGAAG | | | |
| | | GCGCTCTCTCGAGCAGGTGAAACCACTGGTCAGTTAACAAAGATAATC | | | |
| | | CAGGGACCTCAGGAATCCTTCTCAGATTTTGTGGCCAGAATGACAGAG | | | |
| | | GCAGCAGAGCGTATTTTTGGAGAGTCAGAGCAAGCTGCGCCTCTGATA | | | |
| | | GAACAGCTAATCTATGAGCAAGCCACAAAGGAGTGCCGAGCGGCCAT | | | |
| | | AGCCCCAAGAAAGAACAAAGGCTTACAAGACTGGCTCAGGGTCTGTCG | | | |
| | | AGAGCTTGGGGGACCTCTCAGCAATGCAGGTTTAGCGGCTGCCATCCT | | | |
| | | TCAATCCCAAAACCGCTCCATGGGCAGAAATAATCAGAGGACATGTTTT | | | |
| | | AACTGCGGAAAGCCTGGGCATTTTAAGAAAGATTGCAGAGCTCCAGAT | | | |
| | | AAACAGGGAGGGACTCTCACTCTTTGCTCTAAGTGTGGCAAGGGTTAT | | | |
| | | CATAGAGCTGACCAGTGTCGCTCTGTGAGGGATATAAAGGGCAGAGT | | | |
| | | CCTTCCCCCACCTGATAGTCAATCAGCTGATGTGCCAAAAAACGGGTCA | | | |
| | | TCGGGCCCTCGGTCCCAGGGCCCTCAAAGATATGGGAACCGGTTTGTC | | | |
| | | AGGACCCAGGAAGCAGTCAGAGAGGCGACCCAGGAAGACCCACAAG | | | |
| | | GGTGGACCTGCGTGCCGCCTCCGACTTCCTACTAATGCCTCAAATGAGT | | | |
| | | ATTCAGCCGGTGCCGGTGGAGCCTATACCATCCTTGCCCCCGGGAACC | | | |
| | | ATGGGCCTTATTCTCGGCCGGGGTTCACTCACCTTGCAGGGCTTAGTAG | | | |
| | | TCCACCCTGGAGTTATGGATTGTCAACATTCCCCTGAAATACAGGTCCT | | | |
| | | GTGCTCAAGCCCTAAAGGCGTTTTTTCTATTAGTAAAGGAGATAGGAT | | | |
| | | AGCTCAGCTGCTGCTCCTCCCTGATAATACCAGGGAGAAATTTGCAGG | | | |
| | | ACCTGAGATAAAGAAAATGGGCTCCTCAGGAAATGATTCTGCCTATTT | | | |
| | | GGTTGTATCTTTAAATGATAGACCTAAGCTCCGCCTTAAGATTAACGGA | | | |
| | | AAAGAGTTTGAAGGCATCCTTGATACCGGAGCAGATAAAAGTATAATT | | | |
| | | TCTACACATTGGTGGCCCAAAGCATGGCCCACCACAGAGTCATCTCATT | | | |
| | | CATTACAGGGCCTAGGATATCAATCATGTCCCACTATAAGCTCCATTGC | | | |
| | | CTTGACGTGGGAATCCTCTGAAGGACAGCAAGGGAAATTCATACCTTA | | | |
| | | TGTGCTCCCACTCCCGGTTAACCTCTGGGGAAGGGATATTATGCAGCAT | | | |
| | | TTGGGCCTTATTTTGTCCAATGAAAACGCCCCATCAGGAGGGTATTCAG | | | |
| | | CTAAAGCAAAAAATATCATGGCAAAGATGGGTTATAAAGAAGGAAAA | | | |
| | | GGGTTAGGACATCAAGAACAGGGAAGGATAGAGCCCATCTCACCTAAT | | | |
| | | GGAAACCAAGACAGACAGGGTCTGGGTTTTCCTTAGCGGCCATTGGG | | | |
| | | GCAGCACGACCCATACCATGGAAAACAGGGGACCCAGTGTGGGTTCCT | | | |
| | | CAATGGCACCTATCCTCTGAAAAACTAGAAGCTGTGATTCAACTGGTA | | | |
| | | GAGGAACAATTAAAACTAGGCCATATTGAACCCTCTACCTCACCTTGGA | | | |
| | | ATACTCCAATTTTTGTAATTAAGAAAAAGTCAGGAAAGTGGAGACTGC | | | |
| | | TCCATGACCTCAGAGCCATTAATGAGCAAATGAACTTATTTGGCCCAGT | | | |
| | | ACAGAGGGGTCTCCCTGTACTTTCCGCCTTACCACGTGGCTGGAATTTA | | | |
| | | ATTATTATAGATATTAAAGATTGTTTCTTTTCTATACCTTTGTGTCCAAG | | | |
| | | GGATAGGCCCAGATTTGCCTTTACCATCCCCTCTATTAATCACATGGAA | | | |
| | | CCTGATAAGAGGTATCAATGGAAGGTCTTACCACAGGGAATGTCCAAT | | | |
| | | AGTCCTACAATGTGCCAACTTTATGTGCAAGAAGCTCTTTTGCCAGTGA | | | |
| | | GGGAACAATTCCCCTCTTTAATTTTGCTCCTTTACATGGATGACATCCTC | | | |
| | | CTGTGCCATAAAGACCTTACCATGCTACAAAAGGCATATCCTTTTCTACT | | | |
| | | TAAAACTTTAAGTCAGTGGGGTTTACAGATAGCCACAGAAAAGGTCCA | | | |
| | | AATTTCTGATACAGGACAATTCTTGGGCTCTGTGGTGTCCCCAGATAAG | | | |
| | | ATTGTGCCCCAAAAGGTAGAGATAAGAAGAGATCACCTCCATACCTTA | | | |
| | | AATGATTTTCAAAAGCTGTTGGGAGATATTAATTGGCTCAGACCTTTTT | | | |
| | | TAAAGATTCCTTCCGCTGAGTTAAGGCCTTTGTTTAGTATTTTAGAAGG | | | |
| | | AGATCCTCATATCTCCTCCCCTAGGACTCTTACTCTAGCTGCTAACCAGG | | | |
| | | CCTTACAAAAGGTGGAAAAAGCCTTACAGAATGCACAATTACAACGTA | | | |
| | | TTGAGGATTCGCAGCCTTTCAGTTTGTGTGTCTTTAAGACAGCACAATT | | | |
| | | GCCAACTGCAGTTTTGTGGCAGAATGGGCCATTGTTGTGGATCCATCC | | | |
| | | AAACGTATCCCCAGCTAAAATAATAGATTGGTATCCTGATGCAATTGCA | | | |
| | | CAGCTTGCCCTTAAAGGTCTAAAAGCAGCAATCACCCACTTTGGGCAAA | | | |
| | | GTCCATATCTTTTAATTGTACCTTATACCGCTGCACAGGTTCAAACCTTG | | | |
| | | GCAGCCACATCTAATGATTGGGCAGTTTTAGTTACCTCCTTTTCAGGAA | | | |
| | | AAATAGATAACCATTATCCAAAACATCCAATCTTACAGTTTGCCCAAAA | | | |
| | | TCAATCTGTTGTGTTTCCACAAATAACAGTAAGAAACCCACTTAAAAAT | | | |
| | | GGGATTGTGGTATATACTGATGGATCAAAAACTGGCATAGGTGCCTAT | | | |
| | | GTGGCTAATGGTAAAGTGGTATCCAAACAATATAATGAAAATTCACCTC | | | |
| | | AAGTGGTAGAATGTTTAGTGGTCTTAGAAGTTTTAAAAACCTTTTTAGA | | | |
| | | ACCCCTTAATATTGTGTCAGATTCCTGTTATGTGGTTAATGCAGTAAATC | | | |
| | | TTTTAGAAGTGGCTGGAGTGATTAAGCCTTCCAGTAGAGTTGCCAATAT | | | |
| | | TTTTCAGCAGATACAATTAGTTTTGTTATCTAGAAGATTTCCTGTTTATA | | | |
| | | TTACTCATGTTAGAGCCCATTCAGGCCTACCTGGCCCCATGGCTCTGGG | | | |
| | | AAATAATTTGGCAGATAAGGCCACTAAAGTGGTGGCTGCTGCCCTATC | | | |
| | | ATCCCCGGTAGAGGCTGCAAGAAATTTTCATAACAATTTTCATGTGACG | | | |
| | | GCTGAAACATTACGCAGTCGTTTCTCCTTGACAAGAAAAGAAGCCCGT | | | |
| | | GACATTGTTACTCAATGTCAAAGCTGCTGTGAGTTCTTGCCAGTTCCTC | | | |
| | | ATGTGGGAATTAACCCACGCGGTATTCGACCTCTACAGGTCTGGCAAA | | | |
| | | TGGATGTTACACATGTTTCTTCCTTTGGAAAACTTCAATATCTCCATGTG | | | |
| | | TCCATTGACACATGTTCTGGCATCATGTTTGCTTCTCCATTAACCGGAGA | | | |
| | | AAAAGCCTCACATGTGATTCAACATTGCCTTGAGGCATGGAGTGCTTG | | | |
| | | GGGGAAACCCAGACTCCTTAAGACTGATAATGGACCAGCTTATACGTC | | | |
| | | TCAAAAATTCCAACAGTTCTGCCGTCAGATGGACGTGACCCACCTGACT | | | |
| | | GGACTTCCATACAACCCTCAAGGACAGGGTATTGTTGAGCGTGCGCAT | | | |
| | | CGCACCCTCAAAACCTATCTTATAAAACAGAAGAGGGGAACTTTTGAG | | | |
| | | GAGACTGTACCCCGAGCACCAAGAGTGTCGGTGTCTATGGCACTCTTT | | | |
| | | ACACTCAATTTTTTAAATATTGATGCTCATGGCCATACTGCGGCTGAAC | | | |
| | | GTCATTGTACAGAGCCAGATAGGCCCAATGAGATGGTTAAATGGAAAA | | | |
| | | ATGTCCTTGATAATAAATGGTATGGCCCGGATCCTATTTTGATAAGATC | | | |
| | | CAGGGGAGCTATCTGTGTTTTCCCACAGAATGAAAACAACCCATTTTGG | | | |
| | | ATACCAGAAAGACTCACCCGAAAAATCCAGACTGACCAAGGAAATACT | | | |
| | | AATGTCCCTCGTCTTGGTGATGTCCAGGGCGTCAATAATAAAAAGAGA | | | |
| | | GCAGCGTTGGGGGATAATGTCGACATTTCCACTCCCAATGACGGTGAT | | | |
| | | GTATAATGCTCAAGTAgaacaaacgacccaacacccgtgcgttttattctgtctttttat | | | |
| | | tgccgatcccccggccgctttacttgtacagctcgtccatgccgagagtgatcccggcggcggt | | | |
| | | cacgaactccagcaggaccatgtgatcgcgcttctcgttggggtctttgctcagggcggactg | | | |
| | | ggtgctcaggtaagtatcaaggttacaagacaggtttaaggagaccaatagaaactgggctt | | | |
| | | gtcgagacagagaagactcttgcgtttctgataggcacctattggtcttactgacatccacttt | | | |
| | | gcctttctctccacaggtagtggttgtcgggcagcagcacggggccgtcgccgatgggggtgt | | | |
| | | tctgctggtagtggtcggcgagctgcacgctgccgtcctcgatgttgtggcggatcttgaagtt | | | |
| | | caccttgatgccgttcttctgcttgtcggccatgatatagacgttgtggctgttgtagttgtac | | | |
| | | tccagcttgtgccccaggatgttgccgtcctccttgaagtcgatgcccttcagctcgatgcggt | | | |
| | | tcaccagggtgtcgccctcgaacttcacctcggcgcgggtcttgtagttgccgtcgtccttgaa | | | |
| | | gaagatggtgcgctcctggacgtagccttcgggcatggcggacttgaagaagtcgtgctgcttc | | | |
| | | atgtggtcggggtagcggctgaagcactgcacgccgtaggtcagggtggtcacgagggtgggcc | | | |
| | | agggcacgggcagcttgccggtggtgcagatgaacttcagggtcagcttgccgtaggtggca | | | |
| | | tcgccctcgccctcgccggacacgctgaacttgtggccgtttacgtcgccgtccagctcgacca | | | |
| | | ggatgggcaccaccccggtgaacagctcctcgcccttgctcaccatggtggctttaccaacag | | | |
| | | taccggaatgccaagcttgggtcctgtgttctggcggcaaacccgttgcgaaaaagaacgttc | | | |
| | | acggcgactactgcacttatatacggttctcccccaccctcgggaaaaaggcggagccagta | | | |
| | | cacgacatcactttcccagtttaccccgcgccaccttctctaggcaccggatcaattgccgacc | | | |
| | | cctccccccaacttctcggggactgtgggcgatgtgcgctctgcccTTCTCCTGCTTTTTT | | | |
| | | ACCACTAACTAGGAACTGGGTTTGGCCTTAATTCAGACAGCCTTGGCTC | | | |
| | | TGTCTGGACAGGTCCAGACAACTGACACCATTAACACTTTGTCAGCCTC | | | |
| | | AGTGACTACAGTCATAGATGAACAGGCCTCAGCTAATGTCAAGATACA | | | |
| | | GAGAGGTCTCATGCTGGTTAATCAACTCATAGATCTTGTCCAGATACAA | | | |
| | | CTAGATGTATTATGACAATTAACTCAGCTGGGATGTGAACAAAAGTTTC | | | |
| | | CGGGATTGTGTGTTATTTCCATTCAGTATGTTAAATTTACTAGGACAGC | | | |
| | | TAATTTGTCAAAAAGTCTTTTTCAGTATATGTTACAGAATTGGATGGCT | | | |
| | | GAATTTGAACAGATCCTTCGGGAATTGAGACTTCAGGTCAACTCCACG | | | |
| | | CGCTTGGACCTGTCGCTGACCAAAGGATTACCCAATTGGATCTCCTCAG | | | |
| | | CATTTTCCTTCTTTAAAAAATGGGTGGGATTAATATTATTTGGAGATAC | | | |
| | | ACTTTGCTGTGGATTAGTGTTGCTTCTTTGATTGGTCTGTAAGCTTAAG | | | |
| | | GCCTAAACTAGGAGAGACAAGGTGGTTATTGCCCAGGCGCTTGCAGG | | | |
| | | ACTAGAACATGGAGCTCCCCCTGATATATGGTTATCTATGCTTAGGCAA | | | |
| | | TAGGTCGCTGGCCACTCAGCTCTTACATCTCACGAGGCTAGACTCATTG | | | |
| | | CACGGGATGGAGTGAGTGTGCTTCAGCAGCCCGAGAGAGTTGCACGG | | | |
| | | CTAAGCACTGCAATGGAAAGGCTCTGCGGCATATATGAGCCTATTCTA | | | |
| | | GGGAGACATGTCATCTTTCATGAAGGTTCAGTGTCCTAGTTCCCTTCCC | | | |
| | | CCAGGCAAAACGACACGGGAGCAGGTCAGGGTTGCTCTGGGTAAAAG | | | |
| | | CCTGTGAGCCTAAGAGCTAATCCTGTACATGGCTCCTTTACCTACACAC | | | |
| | | TGGGGATTTGACCTCTATCTCCACTCTCATTAATATGGGTGGCCTATTTG | | | |
| | | CTCTTATTAAAAGAAAAGGGGGAGATGTTGGGAGCCGCGCCCACATTC | | | |
| | | GCCGTTACAAGATGGCGCTGACAGCTGTGTTCTAAGTGGTAAACAAAT | | | |
| | | AATCTGCGCATGTGCCGAGGGTGGTTCTTCACTCTATGTGCTCTGCCTT | | | |
| | | CCCCGTGACGTCAACTCGGCCGATGGGCTGCAGCCAATCAGGGAGTG | | | |
| | | ACACGTCCTAGGCGAAGGAGAATTCTCTTTAATAGGGACGGGGTTTTG | | | |
| | | TTTTCTCTCTCTCTTGCTTCTCGCTCGCTCTTGCTTCTTGCACTCTGGCTC | | | |
| | | CTGAAGATGTAAGCAATAAAGTTTTGCCGCAGAAGATTCTGGTCTGTG | | | |
| | | GTGTTCTTCCTGGCCGGGCGTGAGAACGCGTCTAATAACAGCGGCCGC | | | |
| | | AACAGGGGATCCAGACATGATAAGATACATTGATGAGTTTGGACAAAC | | | |
| | | CACAACTAGAATGCAGTGAAAAAAATGCTTTATTTGTGAAATTTGTGAT | | | |
| | | GCTATTGCTTTATTTGTAACCATTATAAGCTGCAATAAACAAGTTAACA | | | |
| | | ACAACAATTGCATTCATTTTATGTTTCAGGTTCAGGGGGAGGTGTGGG | | | |
| | | AGGTTTTTTCGGATCCTCTAGAGTCGACCTGCAGGCATGCAAGCTTGGC | | | |
| | | GTAATCATGGTCATAGCTGTTTCCTGTGTGAAATTGTTATCCGCTCACA | | | |
| | | ATTCCACACAACATACGAGCCGGAAGCATAAAGTGTAAAGCCTGGGGT | | | |
| | | GCCTAATGAGTGAGCTAACTCACATTAATTGCGTTGCGCTCACTGCCCG | | | |
| | | CTTTCCAGTCGGGAAACCTGTCGTGCCAGCTGCATTAATGAATCGGCCA | | | |
| | | ACGCGCGGGGAGAGGCGGTTTGCGTATTGGGCGCTCTTCCGCTTCCTC | | | |
| | | GCTCACTGACTCGCTGCGCTCGGTCGTTCGGCTGCGGCGAGCGGTATC | | | |
| | | AGCTCACTCAAAGGCGGTAATACGGTTATCCACAGAATCAGGGGATAA | | | |
| | | CGCAGGAAAGAACATGTGAGCAAAAGGCCAGCAAAAGGCCAGGAACC | | | |
| | | GTAAAAAGGCCGCGTTGCTGGCGTTTTTCCATAGGCTCCGCCCCCCTGA | | | |
| | | CGAGCATCACAAAAATCGACGCTCAAGTCAGAGGTGGCGAAACCCGA | | | |
| | | CAGGACTATAAAGATACCAGGCGTTTCCCCCTGGAAGCTCCCTCGTGC | | | |
| | | GCTCTCCTGTTCCGACCCTGCCGCTTACCGGATACCTGTCCGCCTTTCTC | | | |
| | | CCTTCGGGAAGCGTGGCGCTTTCTCATAGCTCACGCTGTAGGTATCTCA | | | |
| | | GTTCGGTGTAGGTCGTTCGCTCCAAGCTGGGCTGTGTGCACGAACCCC | | | |
| | | CCGTTCAGCCCGACCGCTGCGCCTTATCCGGTAACTATCGTCTTGAGTC | | | |
| | | CAACCCGGTAAGACACGACTTATCGCCACTGGCAGCAGCCACTGGTAA | | | |
| | | CAGGATTAGCAGAGCGAGGTATGTAGGCGGTGCTACAGAGTTCTTGA | | | |
| | | AGTGGTGGCCTAACTACGGCTACACTAGAAGGACAGTATTTGGTATCT | | | |
| | | GCGCTCTGCTGAAGCCAGTTACCTTCGGAAAAAGAGTTGGTAGCTCTT | | | |
| | | GATCCGGCAAACAAACCACCGCTGGTAGCGGTGGTTTTTTTGTTTGCAA | | | |
| | | GCAGCAGATTACGCGCAGAAAAAAAGGATCTCAAGAAGATCCTTTGAT | | | |
| | | CTTTTCTACGGGGTCTGACGCTCAGTGGAACGAAAACTCACGTTAAGG | | | |
| | | GATTTTGGTCATGAGATTATCAAAAAGGATCTTCACCTAGATCCTTTTA | | | |
| | | AATTAAAAATGAAGTTTTAAATCAATCTAAAGTATATATGAGTAAACTT | | | |
| | | GGTCTGACAGTTACCAATGCTTAATCAGTGAGGCACCTATCTCAGCGAT | | | |
| | | CTGTCTATTTCGTTCATCCATAGTTGCCTGACTCCCCGTCGTGTAGATAA | | | |
| | | CTACGATACGGGAGGGCTTACCATCTGGCCCCAGTGCTGCAATGATAC | | | |
| | | CGCGAGACCCACGCTCACCGGCTCCAGATTTATCAGCAATAAACCAGC | | | |
| | | CAGCCGGAAGGGCCGAGCGCAGAAGTGGTCCTGCAACTTTATCCGCCT | | | |
| | | CCATCCAGTCTATTAATTGTTGCCGGGAAGCTAGAGTAAGTAGTTCGCC | | | |
| | | AGTTAATAGTTTGCGCAACGTTGTTGCCATTGCTACAGGCATCGTGGTG | | | |
| | | TCACGCTCGTCGTTTGGTATGGCTTCATTCAGCTCCGGTTCCCAACGAT | | | |
| | | CAAGGCGAGTTACATGATCCCCCATGTTGTGCAAAAAAGCGGTTAGCT | | | |
| | | CCTTCGGTCCTCCGATCGTTGTCAGAAGTAAGTTGGCCGCAGTGTTATC | | | |
| | | ACTCATGGTTATGGCAGCACTGCATAATTCTCTTACTGTCATGCCATCC | | | |
| | | GTAAGATGCTTTTCTGTGACTGGTGAGTACTCAACCAAGTCATTCTGAG | | | |
| | | AATAGTGTATGCGGCGACCGAGTTGCTCTTGCCCGGCGTCAATACGGG | | | |
| | | ATAATACCGCGCCACATAGCAGAACTTTAAAAGTGCTCATCATTGGAAA | | | |
| | | ACGTTCTTCGGGGCGAAAACTCTCAAGGATCTTACCGCTGTTGAGATCC | | | |
| | | AGTTCGATGTAACCCACTCGTGCACCCAACTGATCTTCAGCATCTTTTAC | | | |
| | | TTTCACCAGCGTTTCTGGGTGAGCAAAAACAGGAAGGCAAAATGCCGC | | | |
| | | AAAAAAGGGAATAAGGGCGACACGGAAATGTTGAATACTCATACTCTT | | | |
| | | CCTTTTTCAATATTATTGAAGCATTTATCAGGGTTATTGTCTCATGAGCG | | | |
| | | GATACATATTTGAATGTATTTAGAAAAATAAACAAATAGGGGTTCCGC | | | |
| | | GCACATTTCCCCGAAAAGTGCCACCTGACGTCTAAGAAACCATTATTAT | | | |
| | | CATGACATTAACCTATAAAAATAGGCGTATCACGAGGCCCTTTCGTCTC | | | |
| | | GCGCGTTTCGGTGATGACGGTGAAAACCTCTGACACATGCAGCTCCCG | | | |
| | | GAGACGGTCACAGCTTGTCTGTAAGCGGATGCCGGGAGCAGACAAGC | | | |
| | | CCGTCAGGGCGCGTCAGCGGGTGTTGGCGGGTGTCGGGGCTGGCTTA | | | |
| | | ACTATGCGGCATCAGAGCAGATTGTACTGAGAGTGCACCATATGCGGT | | | |
| | | GTGAAATACCGCACAGATGCGTAAGGAGAAAATACCGCATCAGGCGC | | | |
| | | CATTCGCCATTCAGGCTGCGCAACTGTTGGGAAGGGCGATCGGTGCGG | | | |
| | | GCCTCTTCGCTATTACGCCAGCTGGCGAAAGGGGGATGTGCTGCAAGG | | | |
| | | CGATTAAGTTGGGTAACGCCAGGGTTTTCCCAGTCACGACGTTGTAAA | | | |
| | | ACGACGGCCAGT | | | |
| CIS_pCMV_ | PLV10022 | GAATTCGAGCTTGCATGCCTGCAGGTCGTTACATAACTTACGGTAAATG | 360 | N/A | |
| R-U5- | | GCCCGCCTGGCTGACCGCCCAACGACCCCCGCCCATTGACGTCAATAAT | | | |
| IAP- | | GACGTATGTTCCCATAGTAACGCCAATAGGGACTTTCCATTGACGTCAA | | | |
| 92L23- | | TGGGTGGAGTATTTACGGTAAACTGCCCACTTGGCAGTACATCAAGTG | | | |
| delPol_ | | TATCATATGCCAAGTACGCCCCCTATTGACGTCAATGACGGTAAATGGC | | | |
| EF1- | | CCGCCTGGCATTATGCCCAGTACATGACCTTATGGGACTTTCCTACTTG | | | |
| GFPai- | | GCAGTACATCTACGTATTAGTCATCGCTATTACCATGGTGATGCGGTTT | | | |
| LTR_v2 | | TGGCAGTACATCAATGGGCGTGGATAGCGGTTTGACTCACGGGGATTT | | | |
| | | CCAAGTCTCCACCCCATTGACGTCAATGGGAGTTTGTTTTGGCACCAAA | | | |
| | | ATCAACGGGACTTTCCAAAATGTCGTAACAACTCCGCCCCATTGACGCA | | | |
| | | AATGGGCGGTAGGCGTGTACGGTGGGAGGTCTATATAAGCAGAGCTG | | | |
| | | TTTTCTCTCTCTCTTGCTTCTCGCTCGCTCTTGCTTCTTGCACTCTGGCTC | | | |
| | | CTGAAGATGTAAGCAATAAAGTTTTGCCGCAGAAGATTCTGGTCTGTG | | | |
| | | GTGTTCTTCCTGGCCGGGCGTGAGAACGCGTCTAATAACAATTGGTGC | | | |
| | | CGAATTCCGGGACGAGAAAAAAACTCGGGACTGGCGCAAGGAAGATC | | | |
| | | CCTCATTCCAGAACCAGAACTGCGGGTCGCGGTAATAAAGGTTCCCGT | | | |
| | | AAAGCAGACTGTTAAGAAGGATTCAACTGTATGAATTCAGAACTTTTCA | | | |
| | | GCTGGGGAACGAGAGTACCAGTGAGTACAGCTTTACGAGGTAAGTCT | | | |
| | | GATCTTGAACTTTCTAAGGAAATTCAAGACAGTCTATCAGAAGTAAAGT | | | |
| | | GGAATATGTTTGGCCTTGAATTTTTTCTGGTGTTAGGAGCCCTTTTGTTC | | | |
| | | CTTTTCACATGTTATCAAGTGATTAAGATAGGGCTGAAAATTCTAGAGG | | | |
| | | AAATTCAGGACAAGCTATCAGAAGTAAAGCGGGGAGAGAGAGTAGGA | | | |
| | | GCAAAGAGAAAATATGGTACACAAAATAAGTATACAGGCCTTTCCAAG | | | |
| | | GGTCTTGAACCCGAGGAAAAGTTAAGGTTAGGTAGGAATACCTGGAG | | | |
| | | AGAGATTAGAAGAAAAAGAGGAAAAAGGGAAAAGAAGAAAGATCAA | | | |
| | | TTAGCGGAGGTCTCTAGGAGATACTCGTCACTAGATGAGCTCAGGAAG | | | |
| | | CCAGCTCTTAGTAGTTCTGAAGCAGATGAAGAATTCTCCTCTGAGGAA | | | |
| | | ACAGACTGGGAGGAAGAAGCAGCCCATTACCAGCCAGCTAATTGGTCA | | | |
| | | AGAAAAAAGCCAAAAGCGGCTGGCGAAGGCCAGTTTGCTGATTGGCC | | | |
| | | TCAGGGCAGTCGGCTTCAAGGTCCGCCCTATGCGGAGTCCCCGCCCTG | | | |
| | | CGTAGTGCGTCAGCAATGCGCAGAGAGGCAGTGCGCAGAGAGGCAGT | | | |
| | | GCGCAGACTCATTCATTCCCAGAGAGGAACAAAGGAAAATACAACAG | | | |
| | | GCATTTCCGGTCTTTGAAGGAGCCGAGGGTGGGCGTGTCCACGCTCCG | | | |
| | | GTAGAATACTTACAAATTAAAGAAATTGCCGAGTCGGTCCGTAAATAT | | | |
| | | GGAACCAATGCTAATTTTACCTTGGTGCAGTTAGACAGGCTCGCCGGC | | | |
| | | ATGGCACTAACTCCTGCTGACTGGCAAACGGTTGTAAAAGCCGCTCTCC | | | |
| | | CTAGTATGGGCAAATATATGGAATGGAGAGCGCTTTGGCACGAAGCT | | | |
| | | GCACAAGCGCAGGCCCGAGCAAACGCAGCTGCTTTGACTCCAGAGCA | | | |
| | | GAGAGATTGGACTTTTGACTTGTTAACGGGTCAGGGAGCTTATTCTGCT | | | |
| | | GATCAGACAAACTACCATTGGGGAGCTTATGCCCAGATTTCTTCCACGG | | | |
| | | CTATTAGGGCCTGGAAGGCGCTCTCTCGAGCAGGTGAAACCACTGGTC | | | |
| | | AGTTAACAAAGATAATCCAGGGACCTCAGGAATCCTTCTCAGATTTTGT | | | |
| | | GGCCAGAATGACAGAGGCAGCAGAGCGTATTTTTGGAGAGTCAGAGC | | | |
| | | AAGCTGCGCCTCTGATAGAACAGCTAATCTATGAGCAAGCCACAAAGG | | | |
| | | AGTGCCGAGCGGCCATAGCCCCAAGAAAGAACAAAGGCTTACAAGAC | | | |
| | | TGGCTCAGGGTCTGTCGAGAGCTTGGGGGACCTCTCAGCAATGCAGGT | | | |
| | | TTAGCGGCTGCCATCCTTCAATCCCAAAACCGCTCCATGGGCAGAAATA | | | |
| | | ATCAGAGGACATGTTTTAACTGCGGAAAGCCTGGGCATTTTAAGAAAG | | | |
| | | ATTGCAGAGCTCCAGATAAACAGGGAGGGACTCTCACTCTTTGCTCTA | | | |
| | | AGTGTGGCAAGGGTTATCATAGAGCTGACCAGTGTCGCTCTGTGAGGG | | | |
| | | ATATAAAGGGCAGAGTCCTTCCCCCACCTGATAGTCAATCAGCTGATGT | | | |
| | | GCCAAAAAACGGGTCATCGGGCCCTCGGTCCCAGGGCCCTCAAAGATA | | | |
| | | TGGGAACCGGTTTGTCAGGACCCAGGAAGCAGTCAGAGAGGCGACCC | | | |
| | | AGGAAGACCCACAAGGGTGGACCTGCGTGCCGCCTCCGACTTCCTACT | | | |
| | | AATGCCTCAAATGAGTATTCAGCCGGTGCCGGTGGAGCCTATACCATC | | | |
| | | CTTGCCCCCGGGAACCATGGGCCTTATTCTCGGCCGGGGTTCACTCACC | | | |
| | | TTGCAGGGCTTAGTAGTCCACCCTGGAGTTATGGATTGTCAACATTCCC | | | |
| | | CTGAAATACAGGTCCTGTGCTCAAGCCCTAAAGGCGTTTTTTCTATTAG | | | |
| | | TAAAGGAGATAGGATAGCTCAGCTGCTGCTCCTCCCTGATAATACCAG | | | |
| | | GGAGAAATTTGCAGGACCTGAGATAAAGAAAATGGGCTCCTCAGGAA | | | |
| | | ATGATTCTGCCTATTTGGTTGTATCTTTAAATGATAGACCTAAGCTCCGC | | | |
| | | CTTAAGATTAACGGAAAAGAGTTTGAAGGCATCCTTGATACCGGAGCA | | | |
| | | GATAAAAGTATAATTTCTACACATTGGTGGCCCAAAGCATGGCCCACCA | | | |
| | | CAGAGTCATCTCATTCATTACAGGGCCTAGGATATCAATCATGTCCCAC | | | |
| | | TATAAGCTCCATTGCCTTGACGTGGGAATCCTCTGAAGGACAGCAAGG | | | |
| | | GAAATTCATACCTTATGTGCTCCCACTCCCGGTTAACCTCTGGGGAAGG | | | |
| | | GATATTATGCAGCATTTGGGCCTTATTTTGTCCAATGAAAACGCCCCAT | | | |
| | | CAGGAGGGTATTCAGCTAAAGCAAAAAATATCATGGCAAAGATGGGTT | | | |
| | | ATAAAGAAGGAAAAGGGTTAGGACATCAAGAACAGGGAAGGATAGA | | | |
| | | GCCCATCTCACCTAATGGAAACCAAGACAGACAGGGTCTGGGTTTTCC | | | |
| | | TTAGCGTAATGCTCAAGTAgaacaaacgacccaacacccgtgcgttttattctgtcttt | | | |
| | | ttattgccgatcccccggccgctttacttgtacagctcgtccatgccgagagtgatcccggcgg | | | |
| | | cggtcacgaactccagcaggaccatgtgatcgcgcttctcgttggggtctttgctcagggcgg | | | |
| | | actgggtgctcaggtaagtatcaaggttacaagacaggtttaaggagaccaatagaaactgg | | | |
| | | gcttgtcgagacagagaagactcttgcgtttctgataggcacctattggtcttactgacatcca | | | |
| | | ctttgcctttctctccacaggtagtggttgtcgggcagcagcacggggccgtcgccgatgggg | | | |
| | | gtgttctgctggtagtggtcggcgagctgcacgctgccgtcctcgatgttgtggcggatcttga | | | |
| | | agttcaccttgatgccgttcttctgcttgtcggccatgatatagacgttgtggctgttgtagtt | | | |
| | | gtactccagcttgtgccccaggatgttgccgtcctccttgaagtcgatgcccttcagctcgatg | | | |
| | | cggttcaccagggtgtcgccctcgaacttcacctcggcgcgggtcttgtagttgccgtcgtcct | | | |
| | | tgaagaagatggtgcgctcctggacgtagccttcgggcatggcggacttgaagaagtcgtgctg | | | |
| | | cttcatgtggtcggggtagcggctgaagcactgcacgccgtaggtcagggtggtcacgagggtg | | | |
| | | ggccagggcacgggcagcttgccggtggtgcagatgaacttcagggtcagcttgccgtaggt | | | |
| | | ggcatcgccctcgccctcgccggacacgctgaacttgtggccgtttacgtcgccgtccagctcg | | | |
| | | accaggatgggcaccaccccggtgaacagctcctcgcccttgctcaccatggtggctttacca | | | |
| | | acagtaccggaatgccaagcttgggtcctgtgttctggcggcaaacccgttgcgaaaaagaa | | | |
| | | cgttcacggcgactactgcacttatatacggttctcccccaccctcgggaaaaaggcggagcc | | | |
| | | agtacacgacatcactttcccagtttaccccgcgccaccttctctaggcaccggatcaattgcc | | | |
| | | gacccctccccccaacttctcggggactgtgggcgatgtgcgctctgcccTTCTCCTGCTT | | | |
| | | TTTTACCACTAACTAGGAACTGGGTTTGGCCTTAATTCAGACAGCCTTG | | | |
| | | GCTCTGTCTGGACAGGTCCAGACAACTGACACCATTAACACTTTGTCAG | | | |
| | | CCTCAGTGACTACAGTCATAGATGAACAGGCCTCAGCTAATGTCAAGA | | | |
| | | TACAGAGAGGTCTCATGCTGGTTAATCAACTCATAGATCTTGTCCAGAT | | | |
| | | ACAACTAGATGTATTATGACAATTAACTCAGCTGGGATGTGAACAAAA | | | |
| | | GTTTCCGGGATTGTGTGTTATTTCCATTCAGTATGTTAAATTTACTAGGA | | | |
| | | CAGCTAATTTGTCAAAAAGTCTTTTTCAGTATATGTTACAGAATTGGAT | | | |
| | | GGCTGAATTTGAACAGATCCTTCGGGAATTGAGACTTCAGGTCAACTC | | | |
| | | CACGCGCTTGGACCTGTCGCTGACCAAAGGATTACCCAATTGGATCTCC | | | |
| | | TCAGCATTTTCCTTCTTTAAAAAATGGGTGGGATTAATATTATTTGGAG | | | |
| | | ATACACTTTGCTGTGGATTAGTGTTGCTTCTTTGATTGGTCTGTAAGCTT | | | |
| | | AAGGCCTAAACTAGGAGAGACAAGGTGGTTATTGCCCAGGCGCTTGC | | | |
| | | AGGACTAGAACATGGAGCTCCCCCTGATATATGGTTATCTATGCTTAGG | | | |
| | | CAATAGGTCGCTGGCCACTCAGCTCTTACATCTCACGAGGCTAGACTCA | | | |
| | | TTGCACGGGATGGAGTGAGTGTGCTTCAGCAGCCCGAGAGAGTTGCA | | | |
| | | CGGCTAAGCACTGCAATGGAAAGGCTCTGCGGCATATATGAGCCTATT | | | |
| | | CTAGGGAGACATGTCATCTTTCATGAAGGTTCAGTGTCCTAGTTCCCTT | | | |
| | | CCCCCAGGCAAAACGACACGGGAGCAGGTCAGGGTTGCTCTGGGTAA | | | |
| | | AAGCCTGTGAGCCTAAGAGCTAATCCTGTACATGGCTCCTTTACCTACA | | | |
| | | CACTGGGGATTTGACCTCTATCTCCACTCTCATTAATATGGGTGGCCTA | | | |
| | | TTTGCTCTTATTAAAAGAAAAGGGGGAGATGTTGGGAGCCGCGCCCAC | | | |
| | | ATTCGCCGTTACAAGATGGCGCTGACAGCTGTGTTCTAAGTGGTAAAC | | | |
| | | AAATAATCTGCGCATGTGCCGAGGGTGGTTCTTCACTCTATGTGCTCTG | | | |
| | | CCTTCCCCGTGACGTCAACTCGGCCGATGGGCTGCAGCCAATCAGGGA | | | |
| | | GTGACACGTCCTAGGCGAAGGAGAATTCTCTTTAATAGGGACGGGGTT | | | |
| | | TTGTTTTCTCTCTCTCTTGCTTCTCGCTCGCTCTTGCTTCTTGCACTCTGG | | | |
| | | CTCCTGAAGATGTAAGCAATAAAGTTTTGCCGCAGAAGATTCTGGTCT | | | |
| | | GTGGTGTTCTTCCTGGCCGGGCGTGAGAACGCGTCTAATAACAGCGGC | | | |
| | | CGCAACAGGGGATCCAGACATGATAAGATACATTGATGAGTTTGGACA | | | |
| | | AACCACAACTAGAATGCAGTGAAAAAAATGCTTTATTTGTGAAATTTGT | | | |
| | | GATGCTATTGCTTTATTTGTAACCATTATAAGCTGCAATAAACAAGTTA | | | |
| | | ACAACAACAATTGCATTCATTTTATGTTTCAGGTTCAGGGGGAGGTGTG | | | |
| | | GGAGGTTTTTTCGGATCCTCTAGAGTCGACCTGCAGGCATGCAAGCTT | | | |
| | | GGCGTAATCATGGTCATAGCTGTTTCCTGTGTGAAATTGTTATCCGCTC | | | |
| | | ACAATTCCACACAACATACGAGCCGGAAGCATAAAGTGTAAAGCCTGG | | | |
| | | GGTGCCTAATGAGTGAGCTAACTCACATTAATTGCGTTGCGCTCACTGC | | | |
| | | CCGCTTTCCAGTCGGGAAACCTGTCGTGCCAGCTGCATTAATGAATCG | | | |
| | | GCCAACGCGCGGGGAGAGGCGGTTTGCGTATTGGGCGCTCTTCCGCTT | | | |
| | | CCTCGCTCACTGACTCGCTGCGCTCGGTCGTTCGGCTGCGGCGAGCGG | | | |
| | | TATCAGCTCACTCAAAGGCGGTAATACGGTTATCCACAGAATCAGGGG | | | |
| | | ATAACGCAGGAAAGAACATGTGAGCAAAAGGCCAGCAAAAGGCCAGG | | | |
| | | AACCGTAAAAAGGCCGCGTTGCTGGCGTTTTTCCATAGGCTCCGCCCCC | | | |
| | | CTGACGAGCATCACAAAAATCGACGCTCAAGTCAGAGGTGGCGAAACC | | | |
| | | CGACAGGACTATAAAGATACCAGGCGTTTCCCCCTGGAAGCTCCCTCG | | | |
| | | TGCGCTCTCCTGTTCCGACCCTGCCGCTTACCGGATACCTGTCCGCCTTT | | | |
| | | CTCCCTTCGGGAAGCGTGGCGCTTTCTCATAGCTCACGCTGTAGGTATC | | | |
| | | TCAGTTCGGTGTAGGTCGTTCGCTCCAAGCTGGGCTGTGTGCACGAAC | | | |
| | | CCCCCGTTCAGCCCGACCGCTGCGCCTTATCCGGTAACTATCGTCTTGA | | | |
| | | GTCCAACCCGGTAAGACACGACTTATCGCCACTGGCAGCAGCCACTGG | | | |
| | | TAACAGGATTAGCAGAGCGAGGTATGTAGGCGGTGCTACAGAGTTCTT | | | |
| | | GAAGTGGTGGCCTAACTACGGCTACACTAGAAGGACAGTATTTGGTAT | | | |
| | | CTGCGCTCTGCTGAAGCCAGTTACCTTCGGAAAAAGAGTTGGTAGCTC | | | |
| | | TTGATCCGGCAAACAAACCACCGCTGGTAGCGGTGGTTTTTTTGTTTGC | | | |
| | | AAGCAGCAGATTACGCGCAGAAAAAAAGGATCTCAAGAAGATCCTTTG | | | |
| | | ATCTTTTCTACGGGGTCTGACGCTCAGTGGAACGAAAACTCACGTTAA | | | |
| | | GGGATTTTGGTCATGAGATTATCAAAAAGGATCTTCACCTAGATCCTTT | | | |
| | | TAAATTAAAAATGAAGTTTTAAATCAATCTAAAGTATATATGAGTAAAC | | | |
| | | TTGGTCTGACAGTTACCAATGCTTAATCAGTGAGGCACCTATCTCAGCG | | | |
| | | ATCTGTCTATTTCGTTCATCCATAGTTGCCTGACTCCCCGTCGTGTAGAT | | | |
| | | AACTACGATACGGGAGGGCTTACCATCTGGCCCCAGTGCTGCAATGAT | | | |
| | | ACCGCGAGACCCACGCTCACCGGCTCCAGATTTATCAGCAATAAACCA | | | |
| | | GCCAGCCGGAAGGGCCGAGCGCAGAAGTGGTCCTGCAACTTTATCCG | | | |
| | | CCTCCATCCAGTCTATTAATTGTTGCCGGGAAGCTAGAGTAAGTAGTTC | | | |
| | | GCCAGTTAATAGTTTGCGCAACGTTGTTGCCATTGCTACAGGCATCGTG | | | |
| | | GTGTCACGCTCGTCGTTTGGTATGGCTTCATTCAGCTCCGGTTCCCAAC | | | |
| | | GATCAAGGCGAGTTACATGATCCCCCATGTTGTGCAAAAAAGCGGTTA | | | |
| | | GCTCCTTCGGTCCTCCGATCGTTGTCAGAAGTAAGTTGGCCGCAGTGTT | | | |
| | | ATCACTCATGGTTATGGCAGCACTGCATAATTCTCTTACTGTCATGCCAT | | | |
| | | CCGTAAGATGCTTTTCTGTGACTGGTGAGTACTCAACCAAGTCATTCTG | | | |
| | | AGAATAGTGTATGCGGCGACCGAGTTGCTCTTGCCCGGCGTCAATACG | | | |
| | | GGATAATACCGCGCCACATAGCAGAACTTTAAAAGTGCTCATCATTGG | | | |
| | | AAAACGTTCTTCGGGGCGAAAACTCTCAAGGATCTTACCGCTGTTGAG | | | |
| | | ATCCAGTTCGATGTAACCCACTCGTGCACCCAACTGATCTTCAGCATCTT | | | |
| | | TTACTTTCACCAGCGTTTCTGGGTGAGCAAAAACAGGAAGGCAAAATG | | | |
| | | CCGCAAAAAAGGGAATAAGGGCGACACGGAAATGTTGAATACTCATA | | | |
| | | CTCTTCCTTTTTCAATATTATTGAAGCATTTATCAGGGTTATTGTCTCATG | | | |
| | | AGCGGATACATATTTGAATGTATTTAGAAAAATAAACAAATAGGGGTT | | | |
| | | CCGCGCACATTTCCCCGAAAAGTGCCACCTGACGTCTAAGAAACCATTA | | | |
| | | TTATCATGACATTAACCTATAAAAATAGGCGTATCACGAGGCCCTTTCG | | | |
| | | TCTCGCGCGTTTCGGTGATGACGGTGAAAACCTCTGACACATGCAGCT | | | |
| | | CCCGGAGACGGTCACAGCTTGTCTGTAAGCGGATGCCGGGAGCAGAC | | | |
| | | AAGCCCGTCAGGGCGCGTCAGCGGGTGTTGGCGGGTGTCGGGGCTGG | | | |
| | | CTTAACTATGCGGCATCAGAGCAGATTGTACTGAGAGTGCACCATATG | | | |
| | | CGGTGTGAAATACCGCACAGATGCGTAAGGAGAAAATACCGCATCAG | | | |
| | | GCGCCATTCGCCATTCAGGCTGCGCAACTGTTGGGAAGGGCGATCGGT | | | |
| | | GCGGGCCTCTTCGCTATTACGCCAGCTGGCGAAAGGGGGATGTGCTGC | | | |
| | | AAGGCGATTAAGTTGGGTAACGCCAGGGTTTTCCCAGTCACGACGTTG | | | |
| | | TAAAACGACGGCCAGT | | | |
| CIS_pCMV_ | PLV10024 | GAATTCGAGCTTGCATGCCTGCAGGTCGTTACATAACTTACGGTAAATG | 361 | N/A | |
| R-U5- | | GCCCGCCTGGCTGACCGCCCAACGACCCCCGCCCATTGACGTCAATAAT | | | |
| IAP- | | GACGTATGTTCCCATAGTAACGCCAATAGGGACTTTCCATTGACGTCAA | | | |
| 92L23_ | | TGGGTGGAGTATTTACGGTAAACTGCCCACTTGGCAGTACATCAAGTG | | | |
| EF1- | | TATCATATGCCAAGTACGCCCCCTATTGACGTCAATGACGGTAAATGGC | | | |
| GFPai- | | CCGCCTGGCATTATGCCCAGTACATGACCTTATGGGACTTTCCTACTTG | | | |
| LTR_v2 | | GCAGTACATCTACGTATTAGTCATCGCTATTACCATGGTGATGCGGTTT | | | |
| | | TGGCAGTACATCAATGGGCGTGGATAGCGGTTTGACTCACGGGGATTT | | | |
| | | CCAAGTCTCCACCCCATTGACGTCAATGGGAGTTTGTTTTGGCACCAAA | | | |
| | | ATCAACGGGACTTTCCAAAATGTCGTAACAACTCCGCCCCATTGACGCA | | | |
| | | AATGGGCGGTAGGCGTGTACGGTGGGAGGTCTATATAAGCAGAGCTG | | | |
| | | TTTTCTCTCTCTCTTGCTTCTCGCTCGCTCTTGCTTCTTGCACTCTGGCTC | | | |
| | | CTGAAGATGTAAGCAATAAAGTTTTGCCGCAGAAGATTCTGGTCTGTG | | | |
| | | GTGTTCTTCCTGGCCGGGCGTGAGAACGCGTCTAATAACAATTGGTGC | | | |
| | | CGAATTCCGGGACGAGAAAAAAACTCGGGACTGGCGCAAGGAAGATC | | | |
| | | CCTCATTCCAGAACCAGAACTGCGGGTCGCGGTAATAAAGGTTCCCGT | | | |
| | | AAAGCAGACTGTTAAGAAGGATTCAACTGTATGAATTCAGAACTTTTCA | | | |
| | | GCTGGGGAACGAGAGTACCAGTGAGTACAGCTTTACGAGGTAAGTCT | | | |
| | | GATCTTGAACTTTCTAAGGAAATTCAAGACAGTCTATCAGAAGTAAAGT | | | |
| | | GGAATATGTTTGGCCTTGAATTTTTTCTGGTGTTAGGAGCCCTTTTGTTC | | | |
| | | CTTTTCACATGTTATCAAGTGATTAAGATAGGGCTGAAAATTCTAGAGG | | | |
| | | AAATTCAGGACAAGCTATCAGAAGTAAAGCGGGGAGAGAGAGTAGGA | | | |
| | | GCAAAGAGAAAATATGGTACACAAAATAAGTATACAGGCCTTTCCAAG | | | |
| | | GGTCTTGAACCCGAGGAAAAGTTAAGGTTAGGTAGGAATACCTGGAG | | | |
| | | AGAGATTAGAAGAAAAAGAGGAAAAAGGGAAAAGAAGAAAGATCAA | | | |
| | | TTAGCGGAGGTCTCTAGGAGATACTCGTCACTAGATGAGCTCAGGAAG | | | |
| | | CCAGCTCTTAGTAGTTCTGAAGCAGATGAAGAATTCTCCTCTGAGGAA | | | |
| | | ACAGACTGGGAGGAAGAAGCAGCCCATTACCAGCCAGCTAATTGGTCA | | | |
| | | AGAAAAAAGCCAAAAGCGGCTGGCGAAGGCCAGTTTGCTGATTGGCC | | | |
| | | TCAGGGCAGTCGGCTTCAAGGTCCGCCCTATGCGGAGTCCCCGCCCTG | | | |
| | | CGTAGTGCGTCAGCAATGCGCAGAGAGGCAGTGCGCAGAGAGGCAGT | | | |
| | | GCGCAGACTCATTCATTCCCAGAGAGGAACAAAGGAAAATACAACAG | | | |
| | | GCATTTCCGGTCTTTGAAGGAGCCGAGGGTGGGCGTGTCCACGCTCCG | | | |
| | | GTAGAATACTTACAAATTAAAGAAATTGCCGAGTCGGTCCGTAAATAT | | | |
| | | GGAACCAATGCTAATTTTACCTTGGTGCAGTTAGACAGGCTCGCCGGC | | | |
| | | ATGGCACTAACTCCTGCTGACTGGCAAACGGTTGTAAAAGCCGCTCTCC | | | |
| | | CTAGTATGGGCAAATATATGGAATGGAGAGCGCTTTGGCACGAAGCT | | | |
| | | GCACAAGCGCAGGCCCGAGCAAACGCAGCTGCTTTGACTCCAGAGCA | | | |
| | | GAGAGATTGGACTTTTGACTTGTTAACGGGTCAGGGAGCTTATTCTGCT | | | |
| | | GATCAGACAAACTACCATTGGGGAGCTTATGCCCAGATTTCTTCCACGG | | | |
| | | CTATTAGGGCCTGGAAGGCGCTCTCTCGAGCAGGTGAAACCACTGGTC | | | |
| | | AGTTAACAAAGATAATCCAGGGACCTCAGGAATCCTTCTCAGATTTTGT | | | |
| | | GGCCAGAATGACAGAGGCAGCAGAGCGTATTTTTGGAGAGTCAGAGC | | | |
| | | AAGCTGCGCCTCTGATAGAACAGCTAATCTATGAGCAAGCCACAAAGG | | | |
| | | AGTGCCGAGCGGCCATAGCCCCAAGAAAGAACAAAGGCTTACAAGAC | | | |
| | | TGGCTCAGGGTCTGTCGAGAGCTTGGGGGACCTCTCAGCAATGCAGGT | | | |
| | | TTAGCGGCTGCCATCCTTCAATCCCAAAACCGCTCCATGGGCAGAAATA | | | |
| | | ATCAGAGGACATGTTTTAACTGCGGAAAGCCTGGGCATTTTAAGAAAG | | | |
| | | ATTGCAGAGCTCCAGATAAACAGGGAGGGACTCTCACTCTTTGCTCTA | | | |
| | | AGTGTGGCAAGGGTTATCATAGAGCTGACCAGTGTCGCTCTGTGAGGG | | | |
| | | ATATAAAGGGCAGAGTCCTTCCCCCACCTGATAGTCAATCAGCTGATGT | | | |
| | | GCCAAAAAACGGGTCATCGGGCCCTCGGTCCCAGGGCCCTCAAAGATA | | | |
| | | TGGGAACCGGTTTGTCAGGACCCAGGAAGCAGTCAGAGAGGCGACCC | | | |
| | | AGGAAGACCCACAAGGGTGGACCTGCGTGCCGCCTCCGACTTCCTACT | | | |
| | | AATGCCTCAAATGAGTATTCAGCCGGTGCCGGTGGAGCCTATACCATC | | | |
| | | CTTGCCCCCGGGAACCATGGGCCTTATTCTCGGCCGGGGTTCACTCACC | | | |
| | | TTGCAGGGCTTAGTAGTCCACCCTGGAGTTATGGATTGTCAACATTCCC | | | |
| | | CTGAAATACAGGTCCTGTGCTCAAGCCCTAAAGGCGTTTTTTCTATTAG | | | |
| | | TAAAGGAGATAGGATAGCTCAGCTGCTGCTCCTCCCTGATAATACCAG | | | |
| | | GGAGAAATTTGCAGGACCTGAGATAAAGAAAATGGGCTCCTCAGGAA | | | |
| | | ATGATTCTGCCTATTTGGTTGTATCTTTAAATGATAGACCTAAGCTCCGC | | | |
| | | CTTAAGATTAACGGAAAAGAGTTTGAAGGCATCCTTGATACCGGAGCA | | | |
| | | GATAAAAGTATAATTTCTACACATTGGTGGCCCAAAGCATGGCCCACCA | | | |
| | | CAGAGTCATCTCATTCATTACAGGGCCTAGGATATCAATCATGTCCCAC | | | |
| | | TATAAGCTCCATTGCCTTGACGTGGGAATCCTCTGAAGGACAGCAAGG | | | |
| | | GAAATTCATACCTTATGTGCTCCCACTCCCGGTTAACCTCTGGGGAAGG | | | |
| | | GATATTATGCAGCATTTGGGCCTTATTTTGTCCAATGAAAACGCCCCAT | | | |
| | | CAGGAGGGTATTCAGCTAAAGCAAAAAATATCATGGCAAAGATGGGTT | | | |
| | | ATAAAGAAGGAAAAGGGTTAGGACATCAAGAACAGGGAAGGATAGA | | | |
| | | GCCCATCTCACCTAATGGAAACCAAGACAGACAGGGTCTGGGTTTTCC | | | |
| | | TTAGCGGCCATTGGGGCAGCACGACCCATACCATGGAAAACAGGGGA | | | |
| | | CCCAGTGTGGGTTCCTCAATGGCACCTATCCTCTGAAAAACTAGAAGCT | | | |
| | | GTGATTCAACTGGTAGAGGAACAATTAAAACTAGGCCATATTGAACCC | | | |
| | | TCTACCTCACCTTGGAATACTCCAATTTTTGTAATTAAGAAAAAGTCAG | | | |
| | | GAAAGTGGAGACTGCTCCATGACCTCAGAGCCATTAATGAGCAAATGA | | | |
| | | ACTTATTTGGCCCAGTACAGAGGGGTCTCCCTGTACTTTCCGCCTTACC | | | |
| | | ACGTGGCTGGAATTTAATTATTATAGATATTAAAGATTGTTTCTTTTCTA | | | |
| | | TACCTTTGTGTCCAAGGGATAGGCCCAGATTTGCCTTTACCATCCCCTCT | | | |
| | | ATTAATCACATGGAACCTGATAAGAGGTATCAATGGAAGGTCTTACCA | | | |
| | | CAGGGAATGTCCAATAGTCCTACAATGTGCCAACTTTATGTGCAAGAA | | | |
| | | GCTCTTTTGCCAGTGAGGGAACAATTCCCCTCTTTAATTTTGCTCCTTTA | | | |
| | | CATGGATGACATCCTCCTGTGCCATAAAGACCTTACCATGCTACAAAAG | | | |
| | | GCATATCCTTTTCTACTTAAAACTTTAAGTCAGTGGGGTTTACAGATAG | | | |
| | | CCACAGAAAAGGTCCAAATTTCTGATACAGGACAATTCTTGGGCTCTGT | | | |
| | | GGTGTCCCCAGATAAGATTGTGCCCCAAAAGGTAGAGATAAGAAGAG | | | |
| | | ATCACCTCCATACCTTAAATGATTTTCAAAAGCTGTTGGGAGATATTAA | | | |
| | | TTGGCTCAGACCTTTTTTAAAGATTCCTTCCGCTGAGTTAAGGCCTTTGT | | | |
| | | TTAGTATTTTAGAAGGAGATCCTCATATCTCCTCCCCTAGGACTCTTACT | | | |
| | | CTAGCTGCTAACCAGGCCTTACAAAAGGTGGAAAAAGCCTTACAGAAT | | | |
| | | GCACAATTACAACGTATTGAGGATTCGCAGCCTTTCAGTTTGTGTGTCT | | | |
| | | TTAAGACAGCACAATTGCCAACTGCAGTTTTGTGGCAGAATGGGCCAT | | | |
| | | TGTTGTGGATCCATCCAAACGTATCCCCAGCTAAAATAATAGATTGGTA | | | |
| | | TCCTGATGCAATTGCACAGCTTGCCCTTAAAGGTCTAAAAGCAGCAATC | | | |
| | | ACCCACTTTGGGCAAAGTCCATATCTTTTAATTGTACCTTATACCGCTGC | | | |
| | | ACAGGTTCAAACCTTGGCAGCCACATCTAATGATTGGGCAGTTTTAGTT | | | |
| | | ACCTCCTTTTCAGGAAAAATAGATAACCATTATCCAAAACATCCAATCTT | | | |
| | | ACAGTTTGCCCAAAATCAATCTGTTGTGTTTCCACAAATAACAGTAAGA | | | |
| | | AACCCACTTAAAAATGGGATTGTGGTATATACTGATGGATCAAAAACT | | | |
| | | GGCATAGGTGCCTATGTGGCTAATGGTAAAGTGGTATCCAAACAATAT | | | |
| | | AATGAAAATTCACCTCAAGTGGTAGAATGTTTAGTGGTCTTAGAAGTTT | | | |
| | | TAAAAACCTTTTTAGAACCCCTTAATATTGTGTCAGATTCCTGTTATGTG | | | |
| | | GTTAATGCAGTAAATCTTTTAGAAGTGGCTGGAGTGATTAAGCCTTCCA | | | |
| | | GTAGAGTTGCCAATATTTTTCAGCAGATACAATTAGTTTTGTTATCTAG | | | |
| | | AAGATTTCCTGTTTATATTACTCATGTTAGAGCCCATTCAGGCCTACCTG | | | |
| | | GCCCCATGGCTCTGGGAAATAATTTGGCAGATAAGGCCACTAAAGTGG | | | |
| | | TGGCTGCTGCCCTATCATCCCCGGTAGAGGCTGCAAGAAATTTTCATAA | | | |
| | | CAATTTTCATGTGACGGCTGAAACATTACGCAGTCGTTTCTCCTTGACA | | | |
| | | AGAAAAGAAGCCCGTGACATTGTTACTCAATGTCAAAGCTGCTGTGAG | | | |
| | | TTCTTGCCAGTTCCTCATGTGGGAATTAACCCACGCGGTATTCGACCTC | | | |
| | | TACAGGTCTGGCAAATGGATGTTACACATGTTTCTTCCTTTGGAAAACT | | | |
| | | TCAATATCTCCATGTGTCCATTGACACATGTTCTGGCATCATGTTTGCTT | | | |
| | | CTCCATTAACCGGAGAAAAAGCCTCACATGTGATTCAACATTGCCTTGA | | | |
| | | GGCATGGAGTGCTTGGGGGAAACCCAGACTCCTTAAGACTGATAATG | | | |
| | | GACCAGCTTATACGTCTCAAAAATTCCAACAGTTCTGCCGTCAGATGGA | | | |
| | | CGTGACCCACCTGACTGGACTTCCATACAACCCTCAAGGACAGGGTATT | | | |
| | | GTTGAGCGTGCGCATCGCACCCTCAAAACCTATCTTATAAAACAGAAG | | | |
| | | AGGGGAACTTTTGAGGAGACTGTACCCCGAGCACCAAGAGTGTCGGT | | | |
| | | GTCTATGGCACTCTTTACACTCAATTTTTTAAATATTGATGCTCATGGCC | | | |
| | | ATACTGCGGCTGAACGTCATTGTACAGAGCCAGATAGGCCCAATGAGA | | | |
| | | TGGTTAAATGGAAAAATGTCCTTGATAATAAATGGTATGGCCCGGATC | | | |
| | | CTATTTTGATAAGATCCAGGGGAGCTATCTGTGTTTTCCCACAGAATGA | | | |
| | | AAACAACCCATTTTGGATACCAGAAAGACTCACCCGAAAAATCCAGAC | | | |
| | | TGACCAAGGAAATACTAATGTCCCTCGTCTTGGTGATGTCCAGGGCGT | | | |
| | | CAATAATAAAAAGAGAGCAGCGTTGGGGGATAATGTCGACATTTCCAC | | | |
| | | TCCCAATGACGGTGATGTATAATGCTCAAGTAgaacaaacgacccaacaccc | | | |
| | | gtgcgttttattctgtctttttattgccgatcccccggccgctttacttgtacagctcgtccat | | | |
| | | gccgagagtgatcccggcggcggtcacgaactccagcaggaccatgtgatcgcgcttctcgttg | | | |
| | | gggtctttgctcagggcggactgggtgctcaggtaagtatcaaggttacaagacaggtttaagg | | | |
| | | agaccaatagaaactgggcttgtcgagacagagaagactcttgcgtttctgataggcacctatt | | | |
| | | ggtcttactgacatccactttgcctttctctccacaggtagtggttgtcgggcagcagcacggg | | | |
| | | gccgtcgccgatgggggtgttctgctggtagtggtcggcgagctgcacgctgccgtcctcgatg | | | |
| | | ttgtggcggatcttgaagttcaccttgatgccgttcttctgcttgtcggccatgatatagacgt | | | |
| | | tgtggctgttgtagttgtactccagcttgtgccccaggatgttgccgtcctccttgaagtcgat | | | |
| | | gcccttcagctcgatgcggttcaccagggtgtcgccctcgaacttcacctcggcgcgggtcttg | | | |
| | | tagttgccgtcgtccttgaagaagatggtgcgctcctggacgtagccttcgggcatggcggact | | | |
| | | tgaagaagtcgtgctgcttcatgtggtcggggtagcggctgaagcactgcacgccgtaggtcag | | | |
| | | ggtggtcacgagggtgggccagggcacgggcagcttgccggtggtgcagatgaacttcagggt | | | |
| | | cagcttgccgtaggtggcatcgccctcgccctcgccggacacgctgaacttgtggccgtttacg | | | |
| | | tcgccgtccagctcgaccaggatgggcaccaccccggtgaacagctcctcgcccttgctcacc | | | |
| | | atggtggctttaccaacagtaccggaatgccaagcttgggtcctgtgttctggcggcaaaccc | | | |
| | | gttgcgaaaaagaacgttcacggcgactactgcacttatatacggttctcccccaccctcggg | | | |
| | | aaaaaggcggagccagtacacgacatcactttcccagtttaccccgcgccaccttctctaggc | | | |
| | | accggatcaattgccgacccctccccccaacttctcggggactgtgggcgatgtgcgctctgcc | | | |
| | | cTTCTCCTGCTTTTTTACCACTAACTAGGAACTGGGTTTGGCCTTAATTC | | | |
| | | AGACAGCCTTGGCTCTGTCTGGACAGGTCCAGACAACTGACACCATTA | | | |
| | | ACACTTTGTCAGCCTCAGTGACTACAGTCATAGATGAACAGGCCTCAGC | | | |
| | | TAATGTCAAGATACAGAGAGGTCTCATGCTGGTTAATCAACTCATAGAT | | | |
| | | CTTGTCCAGATACAACTAGATGTATTATGACAATTAACTCAGCTGGGAT | | | |
| | | GTGAACAAAAGTTTCCGGGATTGTGTGTTATTTCCATTCAGTATGTTAA | | | |
| | | ATTTACTAGGACAGCTAATTTGTCAAAAAGTCTTTTTCAGTATATGTTAC | | | |
| | | AGAATTGGATGGCTGAATTTGAACAGATCCTTCGGGAATTGAGACTTC | | | |
| | | AGGTCAACTCCACGCGCTTGGACCTGTCGCTGACCAAAGGATTACCCA | | | |
| | | ATTGGATCTCCTCAGCATTTTCCTTCTTTAAAAAATGGGTGGGATTAAT | | | |
| | | ATTATTTGGAGATACACTTTGCTGTGGATTAGTGTTGCTTCTTTGATTG | | | |
| | | GTCTGTAAGCTTAAGGCCTAAACTAGGAGAGACAAGGTGGTTATTGCC | | | |
| | | CAGGCGCTTGCAGGACTAGAACATGGAGCTCCCCCTGATATATGGTTA | | | |
| | | TCTATGCTTAGGCAATAGGTCGCTGGCCACTCAGCTCTTACATCTCACG | | | |
| | | AGGCTAGACTCATTGCACGGGATGGAGTGAGTGTGCTTCAGCAGCCCG | | | |
| | | AGAGAGTTGCACGGCTAAGCACTGCAATGGAAAGGCTCTGCGGCATA | | | |
| | | TATGAGCCTATTCTAGGGAGACATGTCATCTTTCATGAAGGTTCAGTGT | | | |
| | | CCTAGTTCCCTTCCCCCAGGCAAAACGACACGGGAGCAGGTCAGGGTT | | | |
| | | GCTCTGGGTAAAAGCCTGTGAGCCTAAGAGCTAATCCTGTACATGGCT | | | |
| | | CCTTTACCTACACACTGGGGATTTGACCTCTATCTCCACTCTCATTAATA | | | |
| | | TGGGTGGCCTATTTGCTCTTATTAAAAGAAAAGGGGGAGATGTTGGGA | | | |
| | | GCCGCGCCCACATTCGCCGTTACAAGATGGCGCTGACAGCTGTGTTCT | | | |
| | | AAGTGGTAAACAAATAATCTGCGCATGTGCCGAGGGTGGTTCTTCACT | | | |
| | | CTATGTGCTCTGCCTTCCCCGTGACGTCAACTCGGCCGATGGGCTGCAG | | | |
| | | CCAATCAGGGAGTGACACGTCCTAGGCGAAGGAGAATTCTCTTTAATA | | | |
| | | GGGACGGGGTTTTGTTTTCTCTCTCTCTTGCTTCTCGCTCGCTCTTGCTT | | | |
| | | CTTGCACTCTGGCTCCTGAAGATGTAAGCAATAAAGTTTTGCCGCAGAA | | | |
| | | GATTCTGGTCTGTGGTGTTCTTCCTGGCCGGGCGTGAGAACGCGTCTA | | | |
| | | ATAACAGCGGCCGCAACAGGGGATCCAGACATGATAAGATACATTGAT | | | |
| | | GAGTTTGGACAAACCACAACTAGAATGCAGTGAAAAAAATGCTTTATT | | | |
| | | TGTGAAATTTGTGATGCTATTGCTTTATTTGTAACCATTATAAGCTGCAA | | | |
| | | TAAACAAGTTAACAACAACAATTGCATTCATTTTATGTTTCAGGTTCAG | | | |
| | | GGGGAGGTGTGGGAGGTTTTTTCGGATCCTCTAGAGTCGACCTGCAGG | | | |
| | | CATGCAAGCTTGGCGTAATCATGGTCATAGCTGTTTCCTGTGTGAAATT | | | |
| | | GTTATCCGCTCACAATTCCACACAACATACGAGCCGGAAGCATAAAGT | | | |
| | | GTAAAGCCTGGGGTGCCTAATGAGTGAGCTAACTCACATTAATTGCGT | | | |
| | | TGCGCTCACTGCCCGCTTTCCAGTCGGGAAACCTGTCGTGCCAGCTGCA | | | |
| | | TTAATGAATCGGCCAACGCGCGGGGAGAGGCGGTTTGCGTATTGGGC | | | |
| | | GCTCTTCCGCTTCCTCGCTCACTGACTCGCTGCGCTCGGTCGTTCGGCT | | | |
| | | GCGGCGAGCGGTATCAGCTCACTCAAAGGCGGTAATACGGTTATCCAC | | | |
| | | AGAATCAGGGGATAACGCAGGAAAGAACATGTGAGCAAAAGGCCAGC | | | |
| | | AAAAGGCCAGGAACCGTAAAAAGGCCGCGTTGCTGGCGTTTTTCCATA | | | |
| | | GGCTCCGCCCCCCTGACGAGCATCACAAAAATCGACGCTCAAGTCAGA | | | |
| | | GGTGGCGAAACCCGACAGGACTATAAAGATACCAGGCGTTTCCCCCTG | | | |
| | | GAAGCTCCCTCGTGCGCTCTCCTGTTCCGACCCTGCCGCTTACCGGATA | | | |
| | | CCTGTCCGCCTTTCTCCCTTCGGGAAGCGTGGCGCTTTCTCATAGCTCAC | | | |
| | | GCTGTAGGTATCTCAGTTCGGTGTAGGTCGTTCGCTCCAAGCTGGGCT | | | |
| | | GTGTGCACGAACCCCCCGTTCAGCCCGACCGCTGCGCCTTATCCGGTAA | | | |
| | | CTATCGTCTTGAGTCCAACCCGGTAAGACACGACTTATCGCCACTGGCA | | | |
| | | GCAGCCACTGGTAACAGGATTAGCAGAGCGAGGTATGTAGGCGGTGC | | | |
| | | TACAGAGTTCTTGAAGTGGTGGCCTAACTACGGCTACACTAGAAGGAC | | | |
| | | AGTATTTGGTATCTGCGCTCTGCTGAAGCCAGTTACCTTCGGAAAAAGA | | | |
| | | GTTGGTAGCTCTTGATCCGGCAAACAAACCACCGCTGGTAGCGGTGGT | | | |
| | | TTTTTTGTTTGCAAGCAGCAGATTACGCGCAGAAAAAAAGGATCTCAA | | | |
| | | GAAGATCCTTTGATCTTTTCTACGGGGTCTGACGCTCAGTGGAACGAA | | | |
| | | AACTCACGTTAAGGGATTTTGGTCATGAGATTATCAAAAAGGATCTTCA | | | |
| | | CCTAGATCCTTTTAAATTAAAAATGAAGTTTTAAATCAATCTAAAGTATA | | | |
| | | TATGAGTAAACTTGGTCTGACAGTTACCAATGCTTAATCAGTGAGGCAC | | | |
| | | CTATCTCAGCGATCTGTCTATTTCGTTCATCCATAGTTGCCTGACTCCCC | | | |
| | | GTCGTGTAGATAACTACGATACGGGAGGGCTTACCATCTGGCCCCAGT | | | |
| | | GCTGCAATGATACCGCGAGACCCACGCTCACCGGCTCCAGATTTATCA | | | |
| | | GCAATAAACCAGCCAGCCGGAAGGGCCGAGCGCAGAAGTGGTCCTGC | | | |
| | | AACTTTATCCGCCTCCATCCAGTCTATTAATTGTTGCCGGGAAGCTAGA | | | |
| | | GTAAGTAGTTCGCCAGTTAATAGTTTGCGCAACGTTGTTGCCATTGCTA | | | |
| | | CAGGCATCGTGGTGTCACGCTCGTCGTTTGGTATGGCTTCATTCAGCTC | | | |
| | | CGGTTCCCAACGATCAAGGCGAGTTACATGATCCCCCATGTTGTGCAA | | | |
| | | AAAAGCGGTTAGCTCCTTCGGTCCTCCGATCGTTGTCAGAAGTAAGTTG | | | |
| | | GCCGCAGTGTTATCACTCATGGTTATGGCAGCACTGCATAATTCTCTTA | | | |
| | | CTGTCATGCCATCCGTAAGATGCTTTTCTGTGACTGGTGAGTACTCAAC | | | |
| | | CAAGTCATTCTGAGAATAGTGTATGCGGCGACCGAGTTGCTCTTGCCC | | | |
| | | GGCGTCAATACGGGATAATACCGCGCCACATAGCAGAACTTTAAAAGT | | | |
| | | GCTCATCATTGGAAAACGTTCTTCGGGGCGAAAACTCTCAAGGATCTTA | | | |
| | | CCGCTGTTGAGATCCAGTTCGATGTAACCCACTCGTGCACCCAACTGAT | | | |
| | | CTTCAGCATCTTTTACTTTCACCAGCGTTTCTGGGTGAGCAAAAACAGG | | | |
| | | AAGGCAAAATGCCGCAAAAAAGGGAATAAGGGCGACACGGAAATGTT | | | |
| | | GAATACTCATACTCTTCCTTTTTCAATATTATTGAAGCATTTATCAGGGT | | | |
| | | TATTGTCTCATGAGCGGATACATATTTGAATGTATTTAGAAAAATAAAC | | | |
| | | AAATAGGGGTTCCGCGCACATTTCCCCGAAAAGTGCCACCTGACGTCT | | | |
| | | AAGAAACCATTATTATCATGACATTAACCTATAAAAATAGGCGTATCAC | | | |
| | | GAGGCCCTTTCGTCTCGCGCGTTTCGGTGATGACGGTGAAAACCTCTG | | | |
| | | ACACATGCAGCTCCCGGAGACGGTCACAGCTTGTCTGTAAGCGGATGC | | | |
| | | CGGGAGCAGACAAGCCCGTCAGGGCGCGTCAGCGGGTGTTGGCGGGT | | | |
| | | GTCGGGGCTGGCTTAACTATGCGGCATCAGAGCAGATTGTACTGAGAG | | | |
| | | TGCACCATATGCGGTGTGAAATACCGCACAGATGCGTAAGGAGAAAAT | | | |
| | | ACCGCATCAGGCGCCATTCGCCATTCAGGCTGCGCAACTGTTGGGAAG | | | |
| | | GGCGATCGGTGCGGGCCTCTTCGCTATTACGCCAGCTGGCGAAAGGG | | | |
| | | GGATGTGCTGCAAGGCGATTAAGTTGGGTAACGCCAGGGTTTTCCCAG | | | |
| | | TCACGACGTTGTAAAACGACGGCCAGT | | | |
| CIS_pCMV_ | PLV100 | GAATTCGAGCTTGCATGCCTGCAGGTCGTTACATAACTTACGGTAAATG | 362 | N/A | |
| R-U5- | 32 | GCCCGCCTGGCTGACCGCCCAACGACCCCCGCCCATTGACGTCAATAAT | | | |
| PBS*- | | GACGTATGTTCCCATAGTAACGCCAATAGGGACTTTCCATTGACGTCAA | | | |
| IAP- | | TGGGTGGAGTATTTACGGTAAACTGCCCACTTGGCAGTACATCAAGTG | | | |
| 92L23_ | | TATCATATGCCAAGTACGCCCCCTATTGACGTCAATGACGGTAAATGGC | | | |
| EF1- | | CCGCCTGGCATTATGCCCAGTACATGACCTTATGGGACTTTCCTACTTG | | | |
| GFPai- | | GCAGTACATCTACGTATTAGTCATCGCTATTACCATGGTGATGCGGTTT | | | |
| LTR_V2 | | TGGCAGTACATCAATGGGCGTGGATAGCGGTTTGACTCACGGGGATTT | | | |
| | | CCAAGTCTCCACCCCATTGACGTCAATGGGAGTTTGTTTTGGCACCAAA | | | |
| | | ATCAACGGGACTTTCCAAAATGTCGTAACAACTCCGCCCCATTGACGCA | | | |
| | | AATGGGCGGTAGGCGTGTACGGTGGGAGGTCTATATAAGCAGAGCTG | | | |
| | | TTTTCTCTCTCTCTTGCTTCTCGCTCGCTCTTGCTTCTTGCACTCTGGCTC | | | |
| | | CTGAAGATGTAAGCAATAAAGTTTTGCCGCAGAAGATTCTGGTCTGTG | | | |
| | | GTGTTCTTCCTGGCCGGGCGTGAGAACGCGTCTAATAACAATTTAGCG | | | |
| | | ACTAAACACATCACGAGAAAAAAACTCGGGACTGGCGCAAGGAAGAT | | | |
| | | CCCTCATTCCAGAACCAGAACTGCGGGTCGCGGTAATAAAGGTTCCCG | | | |
| | | TAAAGCAGACTGTTAAGAAGGATTCAACTGTATGAATTCAGAACTTTTC | | | |
| | | AGCTGGGGAACGAGAGTACCAGTGAGTACAGCTTTACGAGGTAAGTC | | | |
| | | TGATCTTGAACTTTCTAAGGAAATTCAAGACAGTCTATCAGAAGTAAAG | | | |
| | | TGGAATATGTTTGGCCTTGAATTTTTTCTGGTGTTAGGAGCCCTTTTGTT | | | |
| | | CCTTTTCACATGTTATCAAGTGATTAAGATAGGGCTGAAAATTCTAGAG | | | |
| | | GAAATTCAGGACAAGCTATCAGAAGTAAAGCGGGGAGAGAGAGTAG | | | |
| | | GAGCAAAGAGAAAATATGGTACACAAAATAAGTATACAGGCCTTTCCA | | | |
| | | AGGGTCTTGAACCCGAGGAAAAGTTAAGGTTAGGTAGGAATACCTGG | | | |
| | | AGAGAGATTAGAAGAAAAAGAGGAAAAAGGGAAAAGAAGAAAGATC | | | |
| | | AATTAGCGGAGGTCTCTAGGAGATACTCGTCACTAGATGAGCTCAGGA | | | |
| | | AGCCAGCTCTTAGTAGTTCTGAAGCAGATGAAGAATTCTCCTCTGAGG | | | |
| | | AAACAGACTGGGAGGAAGAAGCAGCCCATTACCAGCCAGCTAATTGG | | | |
| | | TCAAGAAAAAAGCCAAAAGCGGCTGGCGAAGGCCAGTTTGCTGATTG | | | |
| | | GCCTCAGGGCAGTCGGCTTCAAGGTCCGCCCTATGCGGAGTCCCCGCC | | | |
| | | CTGCGTAGTGCGTCAGCAATGCGCAGAGAGGCAGTGCGCAGAGAGGC | | | |
| | | AGTGCGCAGACTCATTCATTCCCAGAGAGGAACAAAGGAAAATACAAC | | | |
| | | AGGCATTTCCGGTCTTTGAAGGAGCCGAGGGTGGGCGTGTCCACGCTC | | | |
| | | CGGTAGAATACTTACAAATTAAAGAAATTGCCGAGTCGGTCCGTAAAT | | | |
| | | ATGGAACCAATGCTAATTTTACCTTGGTGCAGTTAGACAGGCTCGCCG | | | |
| | | GCATGGCACTAACTCCTGCTGACTGGCAAACGGTTGTAAAAGCCGCTC | | | |
| | | TCCCTAGTATGGGCAAATATATGGAATGGAGAGCGCTTTGGCACGAAG | | | |
| | | CTGCACAAGCGCAGGCCCGAGCAAACGCAGCTGCTTTGACTCCAGAGC | | | |
| | | AGAGAGATTGGACTTTTGACTTGTTAACGGGTCAGGGAGCTTATTCTG | | | |
| | | CTGATCAGACAAACTACCATTGGGGAGCTTATGCCCAGATTTCTTCCAC | | | |
| | | GGCTATTAGGGCCTGGAAGGCGCTCTCTCGAGCAGGTGAAACCACTG | | | |
| | | GTCAGTTAACAAAGATAATCCAGGGACCTCAGGAATCCTTCTCAGATTT | | | |
| | | TGTGGCCAGAATGACAGAGGCAGCAGAGCGTATTTTTGGAGAGTCAG | | | |
| | | AGCAAGCTGCGCCTCTGATAGAACAGCTAATCTATGAGCAAGCCACAA | | | |
| | | AGGAGTGCCGAGCGGCCATAGCCCCAAGAAAGAACAAAGGCTTACAA | | | |
| | | GACTGGCTCAGGGTCTGTCGAGAGCTTGGGGGACCTCTCAGCAATGCA | | | |
| | | GGTTTAGCGGCTGCCATCCTTCAATCCCAAAACCGCTCCATGGGCAGA | | | |
| | | AATAATCAGAGGACATGTTTTAACTGCGGAAAGCCTGGGCATTTTAAG | | | |
| | | AAAGATTGCAGAGCTCCAGATAAACAGGGAGGGACTCTCACTCTTTGC | | | |
| | | TCTAAGTGTGGCAAGGGTTATCATAGAGCTGACCAGTGTCGCTCTGTG | | | |
| | | AGGGATATAAAGGGCAGAGTCCTTCCCCCACCTGATAGTCAATCAGCT | | | |
| | | GATGTGCCAAAAAACGGGTCATCGGGCCCTCGGTCCCAGGGCCCTCAA | | | |
| | | AGATATGGGAACCGGTTTGTCAGGACCCAGGAAGCAGTCAGAGAGGC | | | |
| | | GACCCAGGAAGACCCACAAGGGTGGACCTGCGTGCCGCCTCCGACTTC | | | |
| | | CTACTAATGCCTCAAATGAGTATTCAGCCGGTGCCGGTGGAGCCTATA | | | |
| | | CCATCCTTGCCCCCGGGAACCATGGGCCTTATTCTCGGCCGGGGTTCAC | | | |
| | | TCACCTTGCAGGGCTTAGTAGTCCACCCTGGAGTTATGGATTGTCAACA | | | |
| | | TTCCCCTGAAATACAGGTCCTGTGCTCAAGCCCTAAAGGCGTTTTTTCT | | | |
| | | ATTAGTAAAGGAGATAGGATAGCTCAGCTGCTGCTCCTCCCTGATAAT | | | |
| | | ACCAGGGAGAAATTTGCAGGACCTGAGATAAAGAAAATGGGCTCCTC | | | |
| | | AGGAAATGATTCTGCCTATTTGGTTGTATCTTTAAATGATAGACCTAAG | | | |
| | | CTCCGCCTTAAGATTAACGGAAAAGAGTTTGAAGGCATCCTTGATACC | | | |
| | | GGAGCAGATAAAAGTATAATTTCTACACATTGGTGGCCCAAAGCATGG | | | |
| | | CCCACCACAGAGTCATCTCATTCATTACAGGGCCTAGGATATCAATCAT | | | |
| | | GTCCCACTATAAGCTCCATTGCCTTGACGTGGGAATCCTCTGAAGGACA | | | |
| | | GCAAGGGAAATTCATACCTTATGTGCTCCCACTCCCGGTTAACCTCTGG | | | |
| | | GGAAGGGATATTATGCAGCATTTGGGCCTTATTTTGTCCAATGAAAAC | | | |
| | | GCCCCATCAGGAGGGTATTCAGCTAAAGCAAAAAATATCATGGCAAAG | | | |
| | | ATGGGTTATAAAGAAGGAAAAGGGTTAGGACATCAAGAACAGGGAAG | | | |
| | | GATAGAGCCCATCTCACCTAATGGAAACCAAGACAGACAGGGTCTGGG | | | |
| | | TTTTCCTTAGCGGCCATTGGGGCAGCACGACCCATACCATGGAAAACA | | | |
| | | GGGGACCCAGTGTGGGTTCCTCAATGGCACCTATCCTCTGAAAAACTA | | | |
| | | GAAGCTGTGATTCAACTGGTAGAGGAACAATTAAAACTAGGCCATATT | | | |
| | | GAACCCTCTACCTCACCTTGGAATACTCCAATTTTTGTAATTAAGAAAA | | | |
| | | AGTCAGGAAAGTGGAGACTGCTCCATGACCTCAGAGCCATTAATGAGC | | | |
| | | AAATGAACTTATTTGGCCCAGTACAGAGGGGTCTCCCTGTACTTTCCGC | | | |
| | | CTTACCACGTGGCTGGAATTTAATTATTATAGATATTAAAGATTGTTTCT | | | |
| | | TTTCTATACCTTTGTGTCCAAGGGATAGGCCCAGATTTGCCTTTACCATC | | | |
| | | CCCTCTATTAATCACATGGAACCTGATAAGAGGTATCAATGGAAGGTCT | | | |
| | | TACCACAGGGAATGTCCAATAGTCCTACAATGTGCCAACTTTATGTGCA | | | |
| | | AGAAGCTCTTTTGCCAGTGAGGGAACAATTCCCCTCTTTAATTTTGCTCC | | | |
| | | TTTACATGGATGACATCCTCCTGTGCCATAAAGACCTTACCATGCTACA | | | |
| | | AAAGGCATATCCTTTTCTACTTAAAACTTTAAGTCAGTGGGGTTTACAG | | | |
| | | ATAGCCACAGAAAAGGTCCAAATTTCTGATACAGGACAATTCTTGGGC | | | |
| | | TCTGTGGTGTCCCCAGATAAGATTGTGCCCCAAAAGGTAGAGATAAGA | | | |
| | | AGAGATCACCTCCATACCTTAAATGATTTTCAAAAGCTGTTGGGAGATA | | | |
| | | TTAATTGGCTCAGACCTTTTTTAAAGATTCCTTCCGCTGAGTTAAGGCCT | | | |
| | | TTGTTTAGTATTTTAGAAGGAGATCCTCATATCTCCTCCCCTAGGACTCT | | | |
| | | TACTCTAGCTGCTAACCAGGCCTTACAAAAGGTGGAAAAAGCCTTACA | | | |
| | | GAATGCACAATTACAACGTATTGAGGATTCGCAGCCTTTCAGTTTGTGT | | | |
| | | GTCTTTAAGACAGCACAATTGCCAACTGCAGTTTTGTGGCAGAATGGG | | | |
| | | CCATTGTTGTGGATCCATCCAAACGTATCCCCAGCTAAAATAATAGATT | | | |
| | | GGTATCCTGATGCAATTGCACAGCTTGCCCTTAAAGGTCTAAAAGCAG | | | |
| | | CAATCACCCACTTTGGGCAAAGTCCATATCTTTTAATTGTACCTTATACC | | | |
| | | GCTGCACAGGTTCAAACCTTGGCAGCCACATCTAATGATTGGGCAGTTT | | | |
| | | TAGTTACCTCCTTTTCAGGAAAAATAGATAACCATTATCCAAAACATCC | | | |
| | | AATCTTACAGTTTGCCCAAAATCAATCTGTTGTGTTTCCACAAATAACA | | | |
| | | GTAAGAAACCCACTTAAAAATGGGATTGTGGTATATACTGATGGATCA | | | |
| | | AAAACTGGCATAGGTGCCTATGTGGCTAATGGTAAAGTGGTATCCAAA | | | |
| | | CAATATAATGAAAATTCACCTCAAGTGGTAGAATGTTTAGTGGTCTTAG | | | |
| | | AAGTTTTAAAAACCTTTTTAGAACCCCTTAATATTGTGTCAGATTCCTGT | | | |
| | | TATGTGGTTAATGCAGTAAATCTTTTAGAAGTGGCTGGAGTGATTAAG | | | |
| | | CCTTCCAGTAGAGTTGCCAATATTTTTCAGCAGATACAATTAGTTTTGTT | | | |
| | | ATCTAGAAGATTTCCTGTTTATATTACTCATGTTAGAGCCCATTCAGGCC | | | |
| | | TACCTGGCCCCATGGCTCTGGGAAATAATTTGGCAGATAAGGCCACTA | | | |
| | | AAGTGGTGGCTGCTGCCCTATCATCCCCGGTAGAGGCTGCAAGAAATT | | | |
| | | TTCATAACAATTTTCATGTGACGGCTGAAACATTACGCAGTCGTTTCTCC | | | |
| | | TTGACAAGAAAAGAAGCCCGTGACATTGTTACTCAATGTCAAAGCTGC | | | |
| | | TGTGAGTTCTTGCCAGTTCCTCATGTGGGAATTAACCCACGCGGTATTC | | | |
| | | GACCTCTACAGGTCTGGCAAATGGATGTTACACATGTTTCTTCCTTTGG | | | |
| | | AAAACTTCAATATCTCCATGTGTCCATTGACACATGTTCTGGCATCATGT | | | |
| | | TTGCTTCTCCATTAACCGGAGAAAAAGCCTCACATGTGATTCAACATTG | | | |
| | | CCTTGAGGCATGGAGTGCTTGGGGGAAACCCAGACTCCTTAAGACTGA | | | |
| | | TAATGGACCAGCTTATACGTCTCAAAAATTCCAACAGTTCTGCCGTCAG | | | |
| | | ATGGACGTGACCCACCTGACTGGACTTCCATACAACCCTCAAGGACAG | | | |
| | | GGTATTGTTGAGCGTGCGCATCGCACCCTCAAAACCTATCTTATAAAAC | | | |
| | | AGAAGAGGGGAACTTTTGAGGAGACTGTACCCCGAGCACCAAGAGTG | | | |
| | | TCGGTGTCTATGGCACTCTTTACACTCAATTTTTTAAATATTGATGCTCA | | | |
| | | TGGCCATACTGCGGCTGAACGTCATTGTACAGAGCCAGATAGGCCCAA | | | |
| | | TGAGATGGTTAAATGGAAAAATGTCCTTGATAATAAATGGTATGGCCC | | | |
| | | GGATCCTATTTTGATAAGATCCAGGGGAGCTATCTGTGTTTTCCCACAG | | | |
| | | AATGAAAACAACCCATTTTGGATACCAGAAAGACTCACCCGAAAAATC | | | |
| | | CAGACTGACCAAGGAAATACTAATGTCCCTCGTCTTGGTGATGTCCAG | | | |
| | | GGCGTCAATAATAAAAAGAGAGCAGCGTTGGGGGATAATGTCGACAT | | | |
| | | TTCCACTCCCAATGACGGTGATGTATAATGCTCAAGTAgaacaaacgaccc | | | |
| | | aacacccgtgcgttttattctgtctttttattgccgatcccccggccgctttacttgtacagct | | | |
| | | cgtccatgccgagagtgatcccggcggcggtcacgaactccagcaggaccatgtgatcgcgctt | | | |
| | | ctcgttggggtctttgctcagggcggactgggtgctcaggtaagtatcaaggttacaagacagg | | | |
| | | tttaaggagaccaatagaaactgggcttgtcgagacagagaagactcttgcgtttctgataggc | | | |
| | | acctattggtcttactgacatccactttgcctttctctccacaggtagtggttgtcgggcagca | | | |
| | | gcacggggccgtcgccgatgggggtgttctgctggtagtggtcggcgagctgcacgctgccgtc | | | |
| | | ctcgatgttgtggcggatcttgaagttcaccttgatgccgttcttctgcttgtcggccatgata | | | |
| | | tagacgttgtggctgttgtagttgtactccagcttgtgccccaggatgttgccgtcctccttga | | | |
| | | agtcgatgcccttcagctcgatgcggttcaccagggtgtcgccctcgaacttcacctcggcgcg | | | |
| | | ggtcttgtagttgccgtcgtccttgaagaagatggtgcgctcctggacgtagccttcgggcatg | | | |
| | | gcggacttgaagaagtcgtgctgcttcatgtggtcggggtagcggctgaagcactgcacgccgt | | | |
| | | aggtcagggtggtcacgagggtgggccagggcacgggcagcttgccggtggtgcagatgaactt | | | |
| | | cagggtcagcttgccgtaggtggcatcgccctcgccctcgccggacacgctgaacttgtggcc | | | |
| | | gtttacgtcgccgtccagctcgaccaggatgggcaccaccccggtgaacagctcctcgccctt | | | |
| | | gctcaccatggtggctttaccaacagtaccggaatgccaagcttgggtcctgtgttctggcggc | | | |
| | | aaacccgttgcgaaaaagaacgttcacggcgactactgcacttatatacggttctcccccacc | | | |
| | | ctcgggaaaaaggcggagccagtacacgacatcactttcccagtttaccccgcgccaccttct | | | |
| | | ctaggcaccggatcaattgccgacccctccccccaacttctcggggactgtgggcgatgtgcg | | | |
| | | ctctgcccTTCTCCTGCTTTTTTACCACTAACTAGGAACTGGGTTTGGCCTT | | | |
| | | AATTCAGACAGCCTTGGCTCTGTCTGGACAGGTCCAGACAACTGACAC | | | |
| | | CATTAACACTTTGTCAGCCTCAGTGACTACAGTCATAGATGAACAGGCC | | | |
| | | TCAGCTAATGTCAAGATACAGAGAGGTCTCATGCTGGTTAATCAACTCA | | | |
| | | TAGATCTTGTCCAGATACAACTAGATGTATTATGACAATTAACTCAGCT | | | |
| | | GGGATGTGAACAAAAGTTTCCGGGATTGTGTGTTATTTCCATTCAGTAT | | | |
| | | GTTAAATTTACTAGGACAGCTAATTTGTCAAAAAGTCTTTTTCAGTATAT | | | |
| | | GTTACAGAATTGGATGGCTGAATTTGAACAGATCCTTCGGGAATTGAG | | | |
| | | ACTTCAGGTCAACTCCACGCGCTTGGACCTGTCGCTGACCAAAGGATTA | | | |
| | | CCCAATTGGATCTCCTCAGCATTTTCCTTCTTTAAAAAATGGGTGGGATT | | | |
| | | AATATTATTTGGAGATACACTTTGCTGTGGATTAGTGTTGCTTCTTTGAT | | | |
| | | TGGTCTGTAAGCTTAAGGCCTAAACTAGGAGAGACAAGGTGGTTATTG | | | |
| | | CCCAGGCGCTTGCAGGACTAGAACATGGAGCTCCCCCTGATATATGGT | | | |
| | | TATCTATGCTTAGGCAATAGGTCGCTGGCCACTCAGCTCTTACATCTCA | | | |
| | | CGAGGCTAGACTCATTGCACGGGATGGAGTGAGTGTGCTTCAGCAGCC | | | |
| | | CGAGAGAGTTGCACGGCTAAGCACTGCAATGGAAAGGCTCTGCGGCA | | | |
| | | TATATGAGCCTATTCTAGGGAGACATGTCATCTTTCATGAAGGTTCAGT | | | |
| | | GTCCTAGTTCCCTTCCCCCAGGCAAAACGACACGGGAGCAGGTCAGGG | | | |
| | | TTGCTCTGGGTAAAAGCCTGTGAGCCTAAGAGCTAATCCTGTACATGG | | | |
| | | CTCCTTTACCTACACACTGGGGATTTGACCTCTATCTCCACTCTCATTAA | | | |
| | | TATGGGTGGCCTATTTGCTCTTATTAAAAGAAAAGGGGGAGATGTTGG | | | |
| | | GAGCCGCGCCCACATTCGCCGTTACAAGATGGCGCTGACAGCTGTGTT | | | |
| | | CTAAGTGGTAAACAAATAATCTGCGCATGTGCCGAGGGTGGTTCTTCA | | | |
| | | CTCTATGTGCTCTGCCTTCCCCGTGACGTCAACTCGGCCGATGGGCTGC | | | |
| | | AGCCAATCAGGGAGTGACACGTCCTAGGCGAAGGAGAATTCTCTTTAA | | | |
| | | TAGGGACGGGGTTTTGTTTTCTCTCTCTCTTGCTTCTCGCTCGCTCTTGC | | | |
| | | TTCTTGCACTCTGGCTCCTGAAGATGTAAGCAATAAAGTTTTGCCGCAG | | | |
| | | AAGATTCTGGTCTGTGGTGTTCTTCCTGGCCGGGCGTGAGAACGCGTC | | | |
| | | TAATAACAGCGGCCGCAACAGGGGATCCAGACATGATAAGATACATTG | | | |
| | | ATGAGTTTGGACAAACCACAACTAGAATGCAGTGAAAAAAATGCTTTA | | | |
| | | TTTGTGAAATTTGTGATGCTATTGCTTTATTTGTAACCATTATAAGCTGC | | | |
| | | AATAAACAAGTTAACAACAACAATTGCATTCATTTTATGTTTCAGGTTC | | | |
| | | AGGGGGAGGTGTGGGAGGTTTTTTCGGATCCTCTAGAGTCGACCTGCA | | | |
| | | GGCATGCAAGCTTGGCGTAATCATGGTCATAGCTGTTTCCTGTGTGAA | | | |
| | | ATTGTTATCCGCTCACAATTCCACACAACATACGAGCCGGAAGCATAAA | | | |
| | | GTGTAAAGCCTGGGGTGCCTAATGAGTGAGCTAACTCACATTAATTGC | | | |
| | | GTTGCGCTCACTGCCCGCTTTCCAGTCGGGAAACCTGTCGTGCCAGCTG | | | |
| | | CATTAATGAATCGGCCAACGCGCGGGGAGAGGCGGTTTGCGTATTGG | | | |
| | | GCGCTCTTCCGCTTCCTCGCTCACTGACTCGCTGCGCTCGGTCGTTCGG | | | |
| | | CTGCGGCGAGCGGTATCAGCTCACTCAAAGGCGGTAATACGGTTATCC | | | |
| | | ACAGAATCAGGGGATAACGCAGGAAAGAACATGTGAGCAAAAGGCCA | | | |
| | | GCAAAAGGCCAGGAACCGTAAAAAGGCCGCGTTGCTGGCGTTTTTCCA | | | |
| | | TAGGCTCCGCCCCCCTGACGAGCATCACAAAAATCGACGCTCAAGTCA | | | |
| | | GAGGTGGCGAAACCCGACAGGACTATAAAGATACCAGGCGTTTCCCCC | | | |
| | | TGGAAGCTCCCTCGTGCGCTCTCCTGTTCCGACCCTGCCGCTTACCGGA | | | |
| | | TACCTGTCCGCCTTTCTCCCTTCGGGAAGCGTGGCGCTTTCTCATAGCTC | | | |
| | | ACGCTGTAGGTATCTCAGTTCGGTGTAGGTCGTTCGCTCCAAGCTGGG | | | |
| | | CTGTGTGCACGAACCCCCCGTTCAGCCCGACCGCTGCGCCTTATCCGGT | | | |
| | | AACTATCGTCTTGAGTCCAACCCGGTAAGACACGACTTATCGCCACTGG | | | |
| | | CAGCAGCCACTGGTAACAGGATTAGCAGAGCGAGGTATGTAGGCGGT | | | |
| | | GCTACAGAGTTCTTGAAGTGGTGGCCTAACTACGGCTACACTAGAAGG | | | |
| | | ACAGTATTTGGTATCTGCGCTCTGCTGAAGCCAGTTACCTTCGGAAAAA | | | |
| | | GAGTTGGTAGCTCTTGATCCGGCAAACAAACCACCGCTGGTAGCGGTG | | | |
| | | GTTTTTTTGTTTGCAAGCAGCAGATTACGCGCAGAAAAAAAGGATCTC | | | |
| | | AAGAAGATCCTTTGATCTTTTCTACGGGGTCTGACGCTCAGTGGAACG | | | |
| | | AAAACTCACGTTAAGGGATTTTGGTCATGAGATTATCAAAAAGGATCTT | | | |
| | | CACCTAGATCCTTTTAAATTAAAAATGAAGTTTTAAATCAATCTAAAGTA | | | |
| | | TATATGAGTAAACTTGGTCTGACAGTTACCAATGCTTAATCAGTGAGGC | | | |
| | | ACCTATCTCAGCGATCTGTCTATTTCGTTCATCCATAGTTGCCTGACTCC | | | |
| | | CCGTCGTGTAGATAACTACGATACGGGAGGGCTTACCATCTGGCCCCA | | | |
| | | GTGCTGCAATGATACCGCGAGACCCACGCTCACCGGCTCCAGATTTATC | | | |
| | | AGCAATAAACCAGCCAGCCGGAAGGGCCGAGCGCAGAAGTGGTCCTG | | | |
| | | CAACTTTATCCGCCTCCATCCAGTCTATTAATTGTTGCCGGGAAGCTAG | | | |
| | | AGTAAGTAGTTCGCCAGTTAATAGTTTGCGCAACGTTGTTGCCATTGCT | | | |
| | | ACAGGCATCGTGGTGTCACGCTCGTCGTTTGGTATGGCTTCATTCAGCT | | | |
| | | CCGGTTCCCAACGATCAAGGCGAGTTACATGATCCCCCATGTTGTGCAA | | | |
| | | AAAAGCGGTTAGCTCCTTCGGTCCTCCGATCGTTGTCAGAAGTAAGTTG | | | |
| | | GCCGCAGTGTTATCACTCATGGTTATGGCAGCACTGCATAATTCTCTTA | | | |
| | | CTGTCATGCCATCCGTAAGATGCTTTTCTGTGACTGGTGAGTACTCAAC | | | |
| | | CAAGTCATTCTGAGAATAGTGTATGCGGCGACCGAGTTGCTCTTGCCC | | | |
| | | GGCGTCAATACGGGATAATACCGCGCCACATAGCAGAACTTTAAAAGT | | | |
| | | GCTCATCATTGGAAAACGTTCTTCGGGGCGAAAACTCTCAAGGATCTTA | | | |
| | | CCGCTGTTGAGATCCAGTTCGATGTAACCCACTCGTGCACCCAACTGAT | | | |
| | | CTTCAGCATCTTTTACTTTCACCAGCGTTTCTGGGTGAGCAAAAACAGG | | | |
| | | AAGGCAAAATGCCGCAAAAAAGGGAATAAGGGCGACACGGAAATGTT | | | |
| | | GAATACTCATACTCTTCCTTTTTCAATATTATTGAAGCATTTATCAGGGT | | | |
| | | TATTGTCTCATGAGCGGATACATATTTGAATGTATTTAGAAAAATAAAC | | | |
| | | AAATAGGGGTTCCGCGCACATTTCCCCGAAAAGTGCCACCTGACGTCT | | | |
| | | AAGAAACCATTATTATCATGACATTAACCTATAAAAATAGGCGTATCAC | | | |
| | | GAGGCCCTTTCGTCTCGCGCGTTTCGGTGATGACGGTGAAAACCTCTG | | | |
| | | ACACATGCAGCTCCCGGAGACGGTCACAGCTTGTCTGTAAGCGGATGC | | | |
| | | CGGGAGCAGACAAGCCCGTCAGGGCGCGTCAGCGGGTGTTGGCGGGT | | | |
| | | GTCGGGGCTGGCTTAACTATGCGGCATCAGAGCAGATTGTACTGAGAG | | | |
| | | TGCACCATATGCGGTGTGAAATACCGCACAGATGCGTAAGGAGAAAAT | | | |
| | | ACCGCATCAGGCGCCATTCGCCATTCAGGCTGCGCAACTGTTGGGAAG | | | |
| | | GGCGATCGGTGCGGGCCTCTTCGCTATTACGCCAGCTGGCGAAAGGG | | | |
| | | GGATGTGCTGCAAGGCGATTAAGTTGGGTAACGCCAGGGTTTTCCCAG | | | |
| | | TCACGACGTTGTAAAACGACGGCCAGT | | | |

TABLE S5
Exemplary full-length driver and template construct elements

| | | SEQ ID | |
|---|---|---|---|
| name | sequence | NO: | length |
| MusD1 | TGTAGTCTCCCCTCCCCCAGCCTGAAACCTGCTTGCTCGGGGTGGAGCTTCCTGCTCATTCGTTCTGCCACGCCCACTGCTGGAACCTGCGGAGCCACACCCGT | 366 | 7478 |
| | GCACCTTTCTACTGGACCAGAGATTATTCGGCGGGAATCGGGTCCCCTCCCCCTTCCTTCATAACTGGTGTCGCAACAATAAAATTTGAGCTTTGATCAGTATG | | |
| | AAATTGCCTTAGCTCCGTTTCTTCTTTTGCCCCGTCTAGATTCCTCTCTTACAGCTCGAGCGGCCTTCTCAGTCGAACCGTTCACGTTGCGAGCTGCTGGCGGCC | | |
| | GCAACATTTTGGCGCCGGAACTGGGACCTGAAGAATGGCAGAGAGATGCTAAGAGGAACGCTGCATTGGAGCTCCACAGGAAAGGATCTTCGTATCGGACA | | |
| | TCGGAACAACGGACAGGTACACATGCTAGCGCTAGCTTAAAATTTCAGTTTTGTAAAGTGTTGCTAAGGATGCGGTAGGATACGAATTAAGCTTGAATCAGT | | |
| | GCTAACCCAACGCTGGTTCTGCTTGGGTCAGCAGCGTGTTAATCGGAACTAGAAACGGAAACAGGCAGGTTAGCCGCAGCTTTTTAGGAAGCTGCTTAGGTG | | |
| | GAAGAAGAAAGGGTTTAAAGTCATGGATCAGGCGGTTGCCAATAGTTTTCAGGAGTTGTTTCAGGCCAGAGGAGTAAGGCTCGAAGTACAATTAGTAAAAA | | |
| | ATTTTTTAGGTAAGATAGATAGCTGTTGCCCATGGTTCAAGGAAGAAGAAACACTAGATTGTGGAACCTGGGAGAAAGTTGGTGAGGCCTTAAAAATCACTC | | |
| | AGGCAGATAATTTTACCCTAGGCCTCTGGGCGCTCGTAAATGATGCAATAAAAGATGCCACTTCCCCAGGGCTAAGTTGCCCCCAGGCGGAGCTTGTGGTAT | | |
| | CTCAGGAGGAGTGCCTGTCAGAGAGGGCCTCCTCAGAAAAAGATCTTCTTAACTCAAAAATTGATAAATGTGGAAACTCGGATGAAAAACTGATTTTTAACA | | |
| | AAAATCACTCAGATAGAGGAGCTGCCCATTACCTTAATGAGAATTGGTCCTCTTGTGAATCTCCCGCTCCACCTGTAGTCCCCACTTCGGGAGGTGCCACTCAT | | |
| | AGGGACACACGACTAAGCGAGTTAGAGTTTGAGATTAAGCTTCAGAGGCTGACTAATGAGCTTCGGGAACTAAAAAAGATGTCAGAAGCGGAGAAGAGTA | | |
| | ACTCTTCTGTAGTTCACCAGGTGCCGCTAGAAAAGGTTGTGAGTCAGGCTCGTGGGAAAGGACAGAATATGTCTAATACGCTAGCCTTTCCGGTGGTCGAGG | | |
| | TAGTTGATCAACAAGATACTAGGGGCAGACATTACCAGACCTTAGATTTCAAGTTGATAAAAGAGTTAAAGGCGGCTGTTGTGCAATATGGCCCTTCAGCCCC | | |
| | GTTCACTCAAGCATTACTGGACACAGTTGTGGAGTCACACTTAACCCCTTTAGATTGGAAGACTCTTTCTAAGGCTACCCTGTCAGGAGGAGATTTTTTGCTTT | | |
| | GGGATTCTGAATGGCGAGACGCCAGTAAAAAAACTGCTGCTTCTAACGCTCAGGCTGGTAACTCAGACTGGGATAGCAACATGCTTTTAGGAGAGGGCCCTT | | |
| | ATGAGGGACAGACAAATCAGATTGATTTTCCCGTTGCAGTGTACGCGCAAATTGCGACGGCCGCACGCCGTGCTTGGGGAAGGTTGCCAGTCAAAGGAGAG | | |
| | ATTGGTGGAAGTTTAGCTAGCATTCGGCAGAGTTCTGATGAACCATATCAGGATTTTGTGGACAGGCTTTTGATTTCAGCTAGTAGAATTCTTGGAAGTCCGG | | |
| | ACACGGGAAGTCCTTTCGTTATGCAATTGGCTTATGAGAATGCTAATGCAATTTGCCGAGCTGCGATTCAACCGCATAAGGGAACGACAGATTTGGCGGGAT | | |
| | ATGTCCGCCTTTGCGCAGACATCGGGCCTTCCTGCGAGACCTTGCAGGGAACCCACGCGCAGGCAATGTTCTCAAGGAAACGAGGGAAAAATGTATGCTTTA | | |
| | AGTGTGGAGGTTTAGATCATTTTAGAATTGATTGTCCTCAGAACAAGGGTGCCGAGGTTAGACAAACAGGCCGTGGCCCAGGAATATGTCCCCGGTGTGGA | | |
| | AAGGGCCGCCACTGGGCGAAAGATTGTAAGCATAAAACGAGGGTTTTGAGCCGCCCGGTGCCGGGAAACGAGGAAAGGGGTCAGCCCCAGGCCCCGAGTT | | |
| | ACTCAAAGAAGACAGCTTATGGGGCTATAAATCTGCTGCCCAGCCAACAAGATCAGTTCTTGAGCTTGTCAGGTCAAACCCAGGAAATGCAAGACTGGACCT | | |
| | CTGTTCCACTGTCCATGTAGCATTAACCCCAGAAGTGGGAGTCCAAACTCTGCCTACCGGAGTTTTTGGACCACTACCTGTAGGAACCTGTGGTTTTCTCTTAG | | |
| | GACGAAGCAGTTCTATTGTAGAAGGCCTGCAGATTTATCCAGGTGTTATAAGTAATGATTATGAGGGAGAAATTAAAATCATAGCCGCTTGCCCTCGTGGTG | | |
| | CTATAACTATACCCGCTAATCAGAAAATTGCTCAACTTACTTTGATCCCTTTGCGCTGGTCACTATCTAAATTCTTTGAAAATGAAGAAGGACAGAATAACTTTG | | |
| | ACTCCCTTGGCGTAAATTGGGTGAAATCTATCACTAATCAGAGACCTAACCTTAAATTGATTCTTGATGGAAAAAGCTTTGAAGGATTAATAGATACCGGGGC | | |
| | CGATGTAACGATAATTAGAGGGCAGGACTGGCCCTCAAACTGGCCCCTGTCTGTTTCCTTGACTCACCTTCAAGGAATTGGTTATGCCAGTAACCCAAAACGT | | |
| | AGTTCCAAATTGCTAACCTGGAGAGATGAGGATGGAAAATCAGGAAATATTCAGCCGTATGTTATGCCAAATTTGCCTGTAACCCTGTGGGGAAGAGATCTG | | |
| | TTGTCACAGATGGGCGTTATCCTGTGCAGTTCTAAGGAGATGGTGACTGAACAGACGTTCAGGCAGGGACCCCTGCCTGATCGTGGACTAATAAAGAAGGG | | |
| | ACAGAAAATTAAGACCTTTGAGGATCTTAAACCCCACTCTAACGTGAGAGGTTTACAGTATTTTCAGTAGCGGCCACTGTCTTGCCTGCATCCCACGCCGAAA | | |
| | AAATTCAATGGCGTAATGATATTCCGGTATGGGTAGATCAGTGGTCCTTGCCTAAAGAGAAAATAGAGGCCGCTTCTTTGCTAGTGCAGGAGCAGTTAGAAG | | |
| | CAGGACATTTGGTGGAGTCTCACTCTCCCTGGAATACACCCATTTTCATTATCAGGAAGAAATCGGGAAAATGGAGACTATTGCAGGATTTAAGAAAGGTTA | | |
| | ATGAAACCATGGTACTTATGGGAACTTTACAACCGGGGCTTCCCTCCCCAGTAGCCATTCCTAAGGGATATTATAAGATTGTTATAGATTTGAAAGATTGTTTC | | |
| | TTTACCATCCCTTTGCATCCAAAGGATTGTGAGAGATTTGCTTTTAGTGTTCCTTCTGTAAATTTCAAGGAACCCATGAAAAGATATCATTGGACAGTTCTCCCG | | |
| | CAGGGCATGGCTAATAGTCCCACCTTATGTCAAAGGTTTGTGGCAAAGGCAATTCAGCCTGTTAGACAACAATGGCCAAATATTTACATCATCCATTTCACAG | | |
| | ATGATGTCCTGATGGCGGGAAAGGACCCCCAAGATTTGCTTTTGTGTTATGGAGACTTACAAAAGGCCCTGGCTGATAAGGGATTACAAATTGCTTCTGAAA | | |
| | AGATACAAACTCAGGATCCTTATAATTATTTGGGTTTTAGACTCACTGATCAAGCTGTTTTTCCCCAGAAAATTGTTATTCGTAGAGATAACTTAAGGACCTTA | | |
| | AATGATTTTCAAAAATTGTTAGGTGATATAAATTGGCTTCGCCCTTATCTAAAGCTTACTACAGGGGAGTTGAAACCTTTATTTGATATTCTTAAAGGGAGTTC | | |
| | TGATCCCACTTCCCCTAGATCCCTAACCTCAGAAGGGTTGCTGGCCTTACAGCTAGTGGAAAAGGCTATTGAAGAACAGTTTGTCACTTACATAGATTACTCCC | | |
| | TGCCGCTGCACCTGTTAATTTTTAATACGACTCATGTGCCTACGGGATTGCTATGGCAAAAATTTCCTATAATGTGGATACATTCGAGGATTTCTCCCAAACGT | | |
| | AATATCTTGCCATATCACGAAGCAGTGGCTCAGATGATTATCACTGGAAGAAGGCAGGCATTGACTTATTTTGGAAAGGAGCCAGATATCATTGTCCAGCCTT | | |
| | ACAGCGTGAGTCAGGACACTTGGCTGAAACAGCATAGTACAGATTGGTTGCTTGCACAATTAGGGTTTGAAGGAACTCTAGATAGCCACTACCCCCAAGATA | | |
| | GGTTGATAAAATTCTTAAATGTGCATGATATGATATTTCCTAAGATGACTTCCTTACAGCCTTTAAATAATGCTCTGTTGATTTTTACTGATGGCTCCTCTAAAG | | |
| | GGCGAGCTGGATATCTTATTAGTAATCAACAGGTTATCGTAGAGACTCCTGGTCTCTCGGCTCAGCTCGCCGAATTAACAGCAGTACTGAAGGTTTTTCAGTC | | |
| | TGTGCATGAGGCTTTTAATATTTTTACTGACAGTTTATATGTTGCTCAGTCAGTACCCTTACTGGAAACCTGTGGTACGTTTAACTTCAATACGCCGTCAGGATC | | |
| | TTTATTTTCAGAATTACAAAACATCATTCTCGCCCGGAAAAATCCGTTTTATATTGGCCACATACGGTCTCACTCTGGTCTTCCTGGACCTCTGGCAGAGGGTA | | |
| | ATGATCGCATTGACAGAGCTCTAATAGGAGAAGCCTTAGTTTCAGATCGGGTTGCTTTGGCCCAACGTGATCACGAAAGGTTTCATCTTTCTAGCCATACCCT | | |
| | AAGGCTCCGACATAAGATCACAAAGGAGCAAGCGAGAATGATCGTAAAACAATGTCCTAAATGTATTACTTTATCTCCAGTGCCTCATCTAGGAGTTAATCCT | | |
| | AGAGGCCTTATGCCTAATCATATTTGGCAAATGGATATAACCCATTATGCAGAATTTGGAAAACTAAAATATATACATGTTTGCATTGATACTTGTTCAGGATT | | |
| | TCTTTTTGCTTCTCTGCATACAGGAGAAGCTTCAAAAAACGTAATTGATCATTGCCTACAAGCATTTAATGCCATGGGATTGCCTAAACTTATTAAGACAGACA | | |
| | ATGGGCCATCTTATTCCAGTAAAAACTTTATTTCATTCTGTAAAGAATTCGGTATTAATCATAAAACTGGAATTCCTTACAACCCCATGGGACAAGGAATAGTT | | |
| | GAACGTGCTCATCGCACCTTAAAGAATTGGCTTTTTAAGACAAAAGAGGGTCAGCTATATCCCCCAAGGTCACCAAAGGCCCACCTTGCCTTCACTTTATTTGT | | |
| | CCTAAATTTCTTGCACACCGATATCAAGGGCCAGTCTGCAGCGGATCGCCACTGGCATCCAGTTACTTCTAATTCTTATGCATTGGTAAAATGGAAGGACCCCC | | |
| | TGACTAATGAATGGAAGGGTCCAGATCCAGTTCTAATTTGGGGTAGGGGCTCAGTTTGTGTTTTTTCACGAGATGAAGATGGAGCGCGGTGGCTGCCAGAG | | |
| | AGATTAATTCGTCAGATGAACACAGATTCTGACTCTTCTGGTAAGTATCATTCTAAAGACTAAAATTCCTTTTGTGCTTAAAATTCAGCTGAGAGCAACAGCTC | | |
| | TCAAAGCTGTTTGTGTTCTCCAGCTACTCTCTGAACCAGCTCCCGACAGGAGGCCGGAGACTAGCCTCAGCTTTACAATTTGCATTTAAATAAAGTACCTAGAC | | |
| | TTCCCCGAAAGAAGTTCTGCTTTCCTACTTTCTCACTGTCTTTCAAGATTTTGTCTTTCAAGCAGGTAAATCAACATTCCCGAGGCGGACCAGCGGATGTGCATC | | |
| | CCCGCCCCCCTAGAGCACACAGGCGGCAGCTGTTACCCCCAGTCTCAGGACATTTCCAGCATGTGGCTTTCAGTCTGAGTTAAAAATTTAGGTTTACCTAGAG | | |
| | GGCTAGAAGAGTAGATTTTTCTATATCAATAAAGATTGGTTTTTATTTTGGTAGACAGGCTTAGCCCCTTAGCTGACCTCTGGCTTTTCACCCTTGCTGTTACTG | | |
| | CAAGGTGTCCTTAGCTCAATAGGCTGTGGAAAAAACAGGGATGAGGAGGAACGACTTCCAGCTCCTATTTTACCCACAAATCGTGGTGTTATTAACGACATA | | |
| | ATTCTTGCTTAGGCTTTGCTAATTCTGAGGTTGATAATTCTCCTTTAGGAGCTGCACAGCACTCAGAACTGTGCATACTGGTTTGTGATTGTACAAATTCAGTAT | | |
| | GGGCACCGCTTGGTGCAGAGATACTTACTGCAAGGGAAGGTCCGGCTTGACCATTTCTGAGTTTCCTGTGAGATAAACCCGGTTTGAAAGAGGTTGGTACCA | | |
| | AATTTTGGTTAAAAATAAAAAATATTCTCCGGCTCTACCTCGCCTCCCCAAAAGGTACCGAGAGCCACATGTGTGGGTTTTACCCACGGGAGGAATCGGGTCC | | |
| | ATGTCCACCCAAGCCAAGGTTAAAAGCCCACTCATCTACGGATGAGAAAATCATTTGATCACCTCAGTTAAGCGTTGCCTTATTTAACTTAATTAATAGGGGG | | |
| | GAGAGAGATTGGAGACGTACTATTGAAAGGGCAAGCCTTTCACTGCCTCCCACCCAAATAAAAAAGCCAATTGGCCTTGTACTACAAGAGCCGGTCACTCCTT | | |
| | CTCCCTGTTTCCCACCTATCTTCCAAAAATGCGGAGGAATTCAACTTAGTGCTATTTTCACATCGTTCAGTCAAACTTAGCCAGAGTTCCAACGCCCTACTTAAA | | |
| | ATTCAACTAGAAAGTTACCTACCAAGTACTAATTAGCATTATAAAGTCAGAGTCTGCAGCTCCAGGCCTTTCAGTTGTTTACTAGAAAGGACAGTCTTAAGCCA | | |
| | GATACAGTTTACCATAAGAAAGGTTAAAGAATCCCAGTGAAGCAAGTTTTTTCTTTAGCCCTAGATTCCAGGCAGAACTATTGAGCATAGATAATTTTCCCCCC | | |
| | TCAGGCCAGCTTTTTCCTTTTTTTTATTTTGTTAATAATAGGGAGGAGATGTAGTCTCCCCTCCCCCAGCCTGAAACCTGCTTGCTCGGGGTGGAGCTTCCTGCT | | |
| | CATTCGTTCTGCCACGCCCACTGCTGGAACCTGCGGAGCCACACCCGTGCACCTTTCTACTGGACCAGAGATTATTCGGCGGGAATCGGGTCCCCTCCCCCCTT | | |
| | CCTTCATAACTGGTGTCGCAACAATAAAATTTGAGCTTTGATCAGTATGAAATTGCCTTAGCTCCGTTTCTTCTTTTGCCCCGTCTAGATTCCTCTCTTACAGCTC | | |
| | GAGCGGCCTTCTCAGTCGAACCGTTCACGTTGCGAGCTGCTGGCGGCCGCAACA | | |
| MusD2 | TGTAGTCTCCCCTCCCCCAGCCTGAAACCTGCTTGCTCGGGGTGGAGCTTCCTGCTCATTCGTTCTGCCACGCCCACTGCTGGAACCTGCGGAGCCACACCCGT | 367 | 7477 |
| | GCACCTTTCTACTGGACCAGAGATTATTCGGCGGGAATCGGGTCCCCTCCCCCTTCCTTCATAACTGGTGTCGCAACAATAAAATTTGAGCTTTGATCAGTATG | | |
| | AAATTGCCTTAGCTCCGTTTCTTCTTTTGCCCCGTCTAGATTCCTCTCTTACAGCTCGAGCGGCCTTCTCAGTCGAACCGTTCACGTTGCGAGCTGCTGGCGGCC | | |
| | GCAACATTTTGGCGCCGGAACTGGGACCTGAAGAATGGCAGAGAGATGCTAAGAGGAACGCTGCATTGGAGCTCCACAGGAAAGGATCTTCGTATCGGACA | | |
| | TCGGAGCAACGGACAGGTACACATGCTAGCGCTAGCTTAAAATTTCAGTTTTGTAAAGTGTTGCTAAGGATGCGGTAGGATACGAATTAAGCTTGAATCAGT | | |
| | GCTAACCCAACGCTGGTTCTGCTTGGGTCAGCAGCGTGTTAATCGGAACTAGAAACGGAAACAGGCAGGTTAGCCGCAGCTTTTTAGGAAGCTGCTTAGGTG | | |
| | GAAGAAGAAAGGGTTTAAAGTCATGGATCAGGCGGTTGCCAATAGTTTTCAGGAGTTGTTTCAGGCCAGAGGAGTAAGGCTCGAAGTACAATTAGTAAAAA | | |
| | ATTTTTTAGGTAAGATAGATAGCTGTTGCCCATGGTTCAAGGAAGAAGAAACACTAGATTGTGGAACCTGGGAGAAAGTTGGTGAGGCCTTAAAAATCACTC | | |
| | AGGCAGATAATTTTACCCTAGGCCTCTGGGCGCTCGTAAATGATGCAATAAAAGATGCCACTTCCCCAGGGCTAAGTTGCCCCCAGGCGGAGCTTGTGGTAT | | |
| | CTCAGGAGGAGTGCCTGTCAGAGAGGGCCTCCTCAGAAAAAGATCTTCTTAACTCAAAAATTGATAAATGTGGAAACTCGGATGAAAAACTGATTTTTAACA | | |
| | AAAATCACTCAGATAGAGGAGCTGCCCATTACCTTAATGAGAATTGGTCCTCTTGTGAATCTCCCGCTCCACCTGTAGTCCCCACTTCGGGAGGTGCCACTCAT | | |
| | AGGGACACACGACTAAGCGAGTTAGAGTTTGAGATTAAGCTTCAGAGGCTGACTAATGAGCTTCGGGAACTAAAAAAGATGTCAGAAGCGGAGAAGAGTA | | |
| | ACTCTTCTGTAGTTCACCAGGTGCCGCTAGAAAAGGTTGTGAGTCAGGCTCGTGGGAAAGGACAGAATATGTCTAATACGCTAGCCTTTCCGGTGGTCGAGG | | |
| | TAGTTGATCAACAAGATACTAGGGGCAGACATTACCAGACCTTAGATTTCAAGTTGATAAAAGAGTTAAAGGCGGCTGTTGTGCAATATGGCCCTTCAGCCCC | | |
| | GTTCACTCAAGCATTACTGGACACAGTTGTGGAGTCACACTTAACCCCTTTAGATTGGAAGACTCTTTCTAAGGCTACCCTGTCAGGAGGAGATTTTTTGCTTT | | |
| | GGGATTCTGAATGGCGAGACGCCAGTAAAAAAACTGCTGCTTCTAACGCTCAGGCTGGTAACTCAGACTGGGATAGCAACATGCTTTTAGGAGAGGGCCCTT | | |
| | ATGAGGGACAGACAAATCAGATTGATTTTCCCGTTGCAGTGTACGCGCAAATTGCGACGGCCGCACGCCGTGCTTGGGGAAGGTTGCCAGTCAAAGGAGAG | | |
| | ATTGGTGGAAGTTTAGCTAGCATTCGGCAGAGTTCTGATGAACCATATCAGGATTTTGTGGACAGGCTTTTGATTTCAGCTAGTAGAATTCTTGGAAGTCCGG | | |
| | ACACGGGAAGTCCTTTCGTTATGCAATTGGCTTATGAGAATGCTAATGCAATTTGCCGAGCTGCGATTCAACCGCATAAGGGAACGACAGATTTGGCGGGAT | | |
| | ATGTCCGCCTTTGCGCAGACATCGGGCCTTCCTGCGAGACCTTGCAGGGAACCCACGCGCAGGCAATGTTCTCAAGGAAACGAGGGAAAAATGTATGCTTTA | | |
| | AGTGTGGAGGTTTAGATCATTTTAGAATTGATTGTCCTCAGAACAAGGGTGCCGAGGTTAGACAAACAGGCCGTGGCCCAGGAATATGTCCCCGGTGTGGA | | |
| | AAGGGCCGCCACTGGGCGAAAGATTGTAAGCATAAAACGAGGGTTTTGAGCCGCCCGGTGCCGGGAAACGAGGAAAGGGGTCAGCCCCAGGCCCCGAGTT | | |
| | ACTCAAAGAAGACAGCTTATGGGGCTATAAATCTGCTGCCCAGCCAACAAGATCAGTTCTTGAGCTTGTCAGGTCAAACCCAGGAAATGCAAGACTGGACCT | | |
| | CTGTTCCACTGTCCATGTAGCATTAACCCCAGAAGTGGGAGTCCAAACTCTGCCTACCGGAGTTTTTGGACCACTACCTGTAGGAACCTGTGGTTTTCTCTTAG | | |
| | GACGAAGCAGTTCTATTGTAGAAGGCCTGCAGATTTATCCAGGTGTTATAAGTAATGATTATGAGGGAGAAATTAAAATCATAGCCGCTTGCCCTCGTGGTG | | |
| | CTATAACTATACCCGCTAATCAGAAAATTGCTCAACTTACTTTGATCCCTTTGCGCTGGTCACTATCTAAATTCTTTGAAAATGAAGAAGGACAGAATAACTTTG | | |
| | ACTCCCTTGGCGTAAATTGGGTGAAATCTATCACTAATCAGAGACCTAACCTTAAATTGATTCTTGATGGAAAAAGCTTTGAAGGATTAATAGATACCGGGGC | | |
| | CGATGTAACGATAATTAGAGGGCAGGACTGGCCCTCAAACTGGCCCCTGTCTGTTTCCTTGACTCACCTTCAAGGAATTGGTTATGCCAGTAACCCAAAACGT | | |
| | AGTTCCAAATTGCTAACCTGGAGAGATGAGGATGGAAAATCAGGAAATATTCAGCCGTATGTTATGCCAAATTTGCCTGTAACCCTGTGGGGAAGAGATCTG | | |
| | TTGTCACAGATGGGCGTTATCCTGTGCAGTTCTAAGGAGATGGTGACTGAACAGACGTTCAGGCAGGGACCCCTGCCTGATCGTGGACTAATAAAGAAGGG | | |
| | ACAGAAAATTAAGACCTTTGAGGATCTTAAACCCCACTCTAACGTGAGAGGTTTACAGTATTTTCAGTAGCGGCCACTGTCTTGCCTGCATCCCACGCCGAAA | | |
| | AAATTCAATGGCGTAATGATATTCCGGTATGGGTAGATCAGTGGTCCTTGCCTAAAGAGAAAATAGAGGCCGCTTCTTTGCTAGTGCAGGAGCAGTTAGAAG | | |
| | CAGGACATTTGGTGGAGTCTCACTCTCCCTGGAATACACCCATTTTCATTATCAGGAAGAAATCGGGAAAATGGAGACTATTGCAGGATTTAAGAAAGGTTA | | |
| | ATGAAACCATGGTACTTATGGGAACTTTACAACCGGGGCTTCCCTCCCCAGTAGCCATTCCTAAGGGATATTATAAGATTGTTATAGATTTGAAAGATTGTTTC | | |
| | TTTACCATCCCTTTGCATCCAAAGGATTGTGAGAGATTTGCTTTTAGTGTTCCTTCTGTAAATTTCAAGGAACCCATGAAAAGATATCATTGGACAGTTCTCCCG | | |
| | CAGGGCATGGCTAATAGTCCCACCTTATGTCAAAGGTTTGTGGCAAAGGCAATTCAGCCTGTTAGACAACAATGGCCAAATATTTACATCATCCATTTCACAG | | |
| | ATGATGTCCTGATGGCGGGAAAGGACCCCCAAGATTTGCTTTTGTGTTATGGAGACTTACAAAAGGCCCTGGCTGATAAGGGATTACAAATTGCTTCTGAAA | | |
| | AGATACAAACTCAGGATCCTTATAATTATTTGGGTTTTAGACTCACTGATCAAGCTGTTTTTCCCCAGAAAATTGTTATTCGTAGAGATAACTTAAGGACCTTA | | |
| | AATGATTTTCAAAAATTGTTAGGTGATATAAATTGGCTTCGCCCTTATCTAAAGCTTACTACAGGGGAGTTGAAACCTTTATTTGATATTCTTAAAGGGAGTTC | | |
| | TGATCCCACTTCCCCTAGATCCCTAACCTCAGAAGGGTTGCTGGCCTTACAGCTAGTGGAAAAGGCTATTGAAGAACAGTTTGTCACTTACATAGATTACTCCC | | |
| | TGCCGCTGCACCTGTTAATTTTTAATACGACTCATGTGCCTACGGGATTGCTATGGCAAAAATTTCCTATAATGTGGATACATTCGAGGATTTCTCCCAAACGT | | |
| | AATATCTTGCCATATCACGAAGCAGTGGCTCAGATGATTATCACTGGAAGAAGGCAGGCATTGACTTATTTTGGAAAGGAGCCAGATATCATTGTCCAGCCTT | | |
| | ACAGCGTGAGTCAGGACACTTGGCTGAAACAGCATAGTACAGATTGGTTGCTTGCACAATTAGGGTTTGAAGGAACTCTAGATAGCCACTACCCCCAAGATA | | |
| | GGTTGATAAAATTCTTAAATGTGCATGATATGATATTTCCTAAGATGACTTCCTTACAGCCTTTAAATAATGCTCTGTTGATTTTTACTGATGGCTCCTCTAAAG | | |
| | GGCGAGCTGGATATCTTATTAGTAATCAACAGGTTATCGTAGAGACTCCTGGTCTCTCGGCTCAGCTCGCCGAATTAACAGCAGTACTGAAGGTTTTTCAGTC | | |
| | TGTGCATGAGGCTTTTAATATTTTTACTGACAGTTTATATGTTGCTCAGTCAGTACCCTTACTGGAAACCTGTGGTACGTTTAACTTCAATACGCCGTCAGGATC | | |
| | TTTATTTTCAGAATTACAAAACATCATTCTCGCCCGGAAAAATCCGTTTTATATTGGCCACATACGGTCTCACTCTGGTCTTCCTGGACCTCTGGCAGAGGGTA | | |
| | ATGATCGCATTGACAGAGCTCTAATAGGAGAAGCCTTAGTTTCAGATCGGGTTGCTTTGGCCCAACGTGATCACGAAAGGTTTCATCTTTCTAGCCATACCCT | | |
| | AAGGCTCCGACATAAGATCACAAAGGAGCAAGCGAGAATGATCGTAAAACAATGTCCTAAATGTATTACTTTATCTCCAGTGCCTCATCTAGGAGTTAATCCT | | |
| | AGAGGCCTTATGCCTAATCATATTTGGCAAATGGATATAACCCATTATGCAGAATTTGGAAAACTAAAATATATACATGTTTGCATTGATACTTGTTCAGGATT | | |
| | TCTTTTTGCTTCTCTGCATACAGGAGAAGCTTCAAAAAACGTAATTGATCATTGCCTACAAGCATTTAATGCCATGGGATTGCCTAAACTTATTAAGACAGACA | | |
| | ATGGGCCATCTTATTCCAGTAAAAACTTTATTTCATTCTGTAAAGAATTCGGTATTAATCATAAAACTGGAATTCCTTACAACCCCATGGGACAAGGAATAGTT | | |
| | GAACGTGCTCATCGCACCTTAAAGAATTGGCTTTTTAAGACAAAAGAGGGTCAGCTATATCCCCCAAGGTCACCAAAGGCCCACCTTGCCTTCACTTTATTTGT | | |
| | CCTAAATTTCTTGCACACCGATATCAAGGGCCAGTCTGCAGCGGATCGCCACTGGCATCCAGTTACTTCTAATTCTTATGCATTGGTAAAATGGAAGGACCCCC | | |
| | TGACTAATGAATGGAAGGGTCCAGATCCAGTTCTAATTTGGGGTAGGGGCTCAGTTTGTGTTTTTTCACGAGATGAAGATGGAGCGCGGTGGCTGCCAGAG | | |
| | AGATTAATTCGTCAGATGAACACAGATTCTGACTCTTCTGGTAAGTATCATTCTAAAGACTAAAATTCCTTTTGTGCTTAAAATTCAGCTGAGAGCAACAGCTC | | |
| | TCAAAGCTGTTTGTGTTCTCCAGCTACTCTCTGAACCAGCTCCCGACAGGAGGCCGGAGACTAGCCTCAGCTTTACAATTTGCATTTAAATAAAGTACCTAGAC | | |
| | TTCCCCGAAAGAAGTTCTGCTTTCCTACTTTCTCACTGTCTTTCAAGATTTTGTCTTTCAAGCAGGTAAATCAACATTCCCGAGGCGGACCAGCGGATGTGCATC | | |
| | CCCGCCCCCCTAGAGCACACAGGCGGCAGCTGTTACCCCCAGTCTCAGGACATTTCCAGCATGTGGCTTTCAGTCTGAGTTAAAAATTTAGGTTTACCTAGAG | | |
| | GGCTAGAAGAGTAGATTTTTCTATATCAATAAAGATTGGTTTTTATTTTGGTAGACAGGCTTAGCCCCTTAGCTGACCTCTGGCTTTTCACCCTTGCTGTTACTG | | |
| | CAAGGTGTCCTTAGCTCAATAGGCTGTGGAAAAAACAGGGATGAGGAGGAACGACTTCCAGCTCCTATTTTACCCACAAATCGTGGTGTTATTAACGACATA | | |
| | ATTCTTGCTTAGGCTTTGCTAATTCTGAGGTTGATAATTCTCCTTTAGGAGCTGCACAGCACTCAGAACTGTGCATACTGGTTTGTGATTGTACAAATTCAGTAT | | |
| | GGGCACCGCTTGGTGCAGAGATACTTACTGCAAGGGAAGGTCCGGCTTGACCATTTCTGAGTTTCCTGTGAGATAAACCCGGTTTGAAAGAGGTTGGTACCA | | |
| | AATTTTGGTTAAAAATAAAAAATATTCTCCGGCTCTACCTCGCCTCCCCAAAAGGTACCGAGAGCCACATGTGTGGGTTTTACCCACGGGAGGAATCGGGTCC | | |
| | ATGTCCACCCAAGCCAAGGTTAAAAGCCCACTCATCTACGGATGAGAAAATCATTTGATCACCTCAGTTAAGCGTTGCCTTATTTAACTTAATTAATAGGGGG | | |
| | GAGAGAGATTGGAGACGTACTATTGAAAGGGCAAGCCTTTCACTGCCTCCCACCCAAATAAAAAAGCCAATTGGCCTTGTACTACAAGAGCCGGTCACTCCTT | | |
| | CTCCCTGTTTCCCACCTATCTTCCAAAAATGCGGAGGAATTCAACTTAGTGCTATTTTCACATCGTTCAGTCAAACTTAGCCAGAGTTCCAACGCCCTACTTAAA | | |
| | ATTCAACTAGAAAGTTACCTACCAAGTACTAATTAGCATTATAAAGTCAGAGTCTGCAGCTCCAGGCCTTTCAGTTGTTTACTAGAAAGGACAGTCTTAAGCCA | | |
| | GATACAGTTTACCATAAGAAAGGTTAAAGAATCCCAGTGAAGCAAGTTTTTTCTTTAGCCCTAGATTCCAGGCAGAACTATTGAGCATAGATAATTTTCCCCCC | | |
| | TCAGGCCAGCTTTTTCCTTTTTTTTATTTTGTTAATAATAGGGAGGAGATGTAGTCTCCCCTCCCCCAGCCTGAAACCTGCTTGCTCGGGGTGGAGCTTCCTGCT | | |
| | CATTCGTTCTGCCACGCCCACTGCTGGAACCTGCGGAGCCACACCCGTGCACCTTTCTACTGGACCAGAGATTATTCGGCGGGAATCGGGTCCCCTCCCCCTTC | | |
| | CTTCATAACTGGTGTCGCAACAATAAAATTTGAGCTTTGATCAGTATGAAATTGCCTTAGCTCCGTTTCTTCTTTTGCCCCGTCTAGATTCCTCTCTTACAGCTCG | | |
| | AGCGGCCTTCTCAGTCGAACCGTTCACGTTGCGAGCTGCTGGCGGCCGCAACA | | |
| MusD3 | TGTAGTCTCCCCTCCCCCAGCCTGAAACCTGCTTGCTCAGGGTGGAGCTTCCTGCTCATTCGTTCTGCCACGCCCACTGCTGGAACCTGAGGAGCCACACACGT | 368 | 7486 |
| | GCACCTTTCTACTGGACCCGAGATTATTCGGCGGGAATCGGGTCCCCTCCCCCTTCCTTCATAACTAGTGTCCCAACAATAAAATTTGAGCTTTGATCAGAATG | | |
| | AATTTGTCTTAGCTCCGTTTCTTCTTTCGCCCCGTCTAGATTCCTCTCTTACAGCTCGAGTGGCCTTCTCAGTCGAACCGTTCACGTTGCGAGCTGCTGGCGGCC | | |
| | GCAACATTTTGGCGCCAGAACTGGGACCTGAAGAATGGCAGAGAGATGCTAAGAGGAACGCTGCATTGGAGCTCCACAGGAAAGGATCTTCGTATCGGACA | | |
| | TCGGAGCAACGGACCGTATCAGACATCGGAGCAACGGACAGGTACACATGCTAGCGCTAGCTTAAAATTTCAGTTTTGTAAAGTGTTGCTGAGGATGCGGTA | | |
| | GGATACGAATTAAGCTTGAATCAGTGCTAACCCAACGCTGGTTCTGCTTGGGTCAGCAGCGTGTTAATCGGAACTAGAAACGGAAACAGGCAGGTTAGCCGC | | |
| | AGCTTTTTAGGAAGCTGCTTAGGTGGAAGAAGAAAGGGTTTAAAGTCATGGATCAGGCGGTTGCCCATAGTTTTCAGGAGTTGTTTCAGGCCAGAGGAGTA | | |
| | AGGCTTGAAGTACAATTAGTAAAAAATTTTTTAGGTAAGATAGATAGCTGTTGCCCATGGTTCAAGGAAGAAGAAACACTAGATTGTGGAACCTGGGAGAAA | | |
| | GTTGGTGAGGCCTTAAAAATCACTCAGGCAGATAATTTTACCCTAGGCCTCTGGGCGCTCGTAAATGATGCAATAAAAGATGCCACTTCCCCAGGGCTAAGTT | | |
| | GCCCCCAGGCGGAGCTTGTGGTATCTCAGGAGGAGTGCCTGTCAGAGAGGGCCTCCTCAGAAAAAGATCTTCTTAACTCAAAAACTGATAAATGTGGAAACT | | |
| | CGGATGAAAAACTGATTTTTAACAAAAATCACTCAGATAGAGGAGCTGCCCATTACCTTAATGAGAATTGGTCCTCTTGTGAATCTCCCGCTCCACCTGTAGTC | | |
| | CCCACTTCAGGAGGTGCCACTCATAGGGACACACGACTAAGCGAGTTAGAGTTTGAGATTAAGCTTCAGAGGCTGACTAATGAGCTTCGGGAACTAAAAAA | | |
| | GATGTCAGAAGCGGAGAAGAGTAACTCTTCTGTAGTTCACCAGGTGCCGCTAGAAAAGGTTGTGAGTCAGGCTCGTGGGAAAGGACAGAATATATCTAATA | | |
| | CGCTAGCCTTTCCTGTGGTCGAGGTAATTGATCAGCAAGATACTAGGGGCAGACATTACCAGACCTTAGATTTCAAGTTGATAAAAGAGTTAAAGGCGGCTG | | |
| | TTGTGCAATATGGCCCTTCAGCCCCATTCACTCAAGCATTACTGGACACAGTTGTGGAGTCACACTTAACCCCTTTAGATTGGAAGACTCTTTCTAAGGCTACC | | |
| | CTGTCAGGAGGAGATTTTTTGCTTTGGGATTCTGAATGGCGAGACGCCAGTAAGAAAACTGCTGCTTCTAACGCTCAGGCTGGTAATTCAGACTGGGATAGC | | |
| | AACATGCTTTTAGGAGAGGGCCCTTATGAGGGACAGACAAATCAGATTGATTTTCCCGTTGCAGTGTACGCGCAAATTGCGACGGCCGCACGCCGTGCTTGG | | |
| | GGAAGGTTGCCAGTCAAAGGAGAGATTGGTGGAAGTTTAGCTAGCATTCGGCAGAGTTCTGATGAACCATATCAGGATTTTGTGGACAGGCTATTGATTTCA | | |
| | GCTAGTAGAATCCTTGGGAATCCGGACACGGGAAGTCCTTTCGTTATGCAATTGGCTTATGAGAATGCTAATGCAATTTGCCGAGCTGCGATTCAACCGCATA | | |
| | AGGGAACGACAGATTTGGCGGGATATGTCCGCCTTTGCGCAGACATCGGGCCTTCCTGCGAGACCTTGCAGGGAACCCACGTGCAGGCAATGTTCTCAAGG | | |
| | AAACGAGGGAAAAATGTATGCTTTAAGTGTGGAAGTTTAGATCATTTTAGAATTGATTGTCCTCAGAACAAGGGTGCCGAGGTTAGACAAACAGGCCGTGCC | | |
| | CCAGGAATATGTCCCCGGTGTGGGAAGGGCCGCCACTGGGCAAAAGATTGTAAGCATAAAACGAGGGTTTTGAGCCGCCCGGTGCCGGGAAACGAGGAAA | | |
| | GGGGTCAGCCCCAGGCCCCGAGTTACTCAAAGAAGACAGCTTATGGGGCTATAAATCTGCTGCCCAGCCAACAAGATCAGTTCTTGAGCTTGTCAGGTCAAA | | |
| | CCCAGGAAATGCAAGACTGGACCTCTGTTCCACTGTCCATGCAGCATTAACCCCAGAAGTGGGAGTCCAAACTCTGCCTACCAGAGTCTTTGGACCACTACCT | | |
| | GTAGGAACCTGTGGTTTTCTCTTAGGACGAAGCAGTTCTATTGTAGAAGGCCTGCAGATTTATCCAGGTGTTATAAGTAATGATTATGAGGGAGAAATTAAA | | |
| | ATCATAGCCGCTTGCCCTCGTGGTGCTATAACTATACCCGCTAATCAGAAAATTGCTCAACTTACTTTGATCCCTTTGCGCTGGTCACTATCTAAATTCTTTGAA | | |
| | AATGAAGAAGGACAGAATAACTTTGACTCCTCTGGCGTAAATTGGGTGAAATCTATCACTAATCAGAGACCTAACCTTAAATTGATTCTTGATGGAAAAAGCT | | |
| | TTGAAGGATTAATAGATACCGGGGCCGATGTAACGATTATTAGAGGGCAGGACTGGCCCTCAAACTGGCCCCTGTCTGTTTCCTTGACTCACCTTCAAGGAAT | | |
| | TGGTTATGCCAGTAACCCAAAACGTAGTTCCAAATTGCTAACCTGGAGAGATGAGGATGGAAAATCAGGAAATATTCAGCCGTATGTTATGCCAAATTTGCCT | | |
| | GTGACCCTGTGGGGAAGAGATCTGTTGTCACAGATGGGCGTTATCCTGTGCAGTTCTAAGGAGATGGTGACTGAACAGACATTCAGGCAGGGACTCCTGCCT | | |
| | GATCGTGGACTAATAAAGAAGGGACAGAAAATTAAGACTTTTGAGGATTTTAAACCCCACTCTAACGTGAGAGGTTTAAAGTATTTTCAGTAGCGGCCACTG | | |
| | TCTTGCCTGCATCCCACGCCGAAAAAATTCAATGGCGTAATGATATTCCGGTGTGGGTAGATCAGTGGTCTTTACCTAAAGAGAAAATAGAGGCCGCTTCTTT | | |
| | GCTAGTGCAGGAGCAGTTAGAAGCAGGACATTTGGTGGAGTCTCACTCTCCCTGGAATACACCCGTTTTCATTATCAGGAAGAAATCGGGAAAATGGAGACT | | |
| | GTTGCAAGATTTAAGAAAGGTTAATGAAACCATGGTACTTATGGGAACTTTACAACCGGGGCTCCCCTCCCCAGTAGCCATTCCTAAGGGATATTATAAGATT | | |
| | GTTATAGATTTGAAAGATTGTTTCTTTACCATCCCTTTGCATCCAAAGGATTGTGAGAGATTTGCTTTTAGTGTTCCTTCTGTAAATTTCAAGGAACCCATGAAA | | |
| | AGATATCAATGGACAGTTCTCCCGCAGGGCATGGCTAATAGTCCCACCTTATGTCAAAGGTTTGTGGCAAAGGCAATTCAGCCTGTTAGACAACAATGGCCA | | |
| | AATATTTACATCATTCATTTCACAGATGATGTCTTGATGGTGGGAAAGGACCCCCAAGATTTGCTTTTGTGTTATGGAGACTTACGAAAGGCCCTGGCTGATA | | |
| | AGGGATTACAAATTGCTTCTGAAAAGATACAAACTCAGGATCCTTATAATTATTTGGGTTTTAGACTCACTGATCAAGCTGTTTTTCCCCAGAAAATTGTTATTC | | |
| | GTAGAGATAACTTAAGGACCTTAAATGATTTTCAAAAATTGTTAGGTGACATAAATTGGCTTCGCCCCTATCTAAAGCTTACTACAGGGGAGTTGAAACCTTTA | | |
| | TTTGATATTCTTAAAGGGAGTTCTGATCCCACTTCCCCTAGATCCCTAACCTCAGAAGGATTACTGGCCTTACAGCTAGTGGAAAAGGCTATTGAAGAACAGTT | | |
| | TGTCACTTACATAGATTACTCCCTGCCGCTGCACCTGTTAATTTTTAATACGACTCATGTGCCTACGGGATTGCTATGGCAAAAATTTCCTATAATGTGGATACA | | |
| | TTCGAGGATTTCTCCCAAACGTAATATCTTGCCATATCACGAAGCAGTGGCTCAGATGATTATCACTGGAAGAAAGCAGGCATTGACTTATTTTGGAAAGGAG | | |
| | CCAGATATCATTGTCCAGCCTTACAGCGTGAGTCAGGACACTTGGCTGAAACAGCATAGTACAGATTGGTTGCTTGCACAATTAGGGTTTGAAGGAACTATA | | |
| | GATAGCCACTACCCCCAAGATAGGTTGATAAAATTCTTAAATGTGCATGATATGATATTTCCTAAGATGACTTCCTTACAGCCTTTAAATAATGCTCTATTGATT | | |
| | TTTACTGATGGCTCCTCTAAAGGGCGAGCTGGATATCTTATTAGTAATCAACAGGTTATCGTAGAGACTCCTGGTCTCTCGGCTCAGCTCGCCGAATTAACAG | | |
| | CAGTACTGAAGGTTTTTCAGTCTGTACATGAGGCTTTTAATATTTTTACTGACAGTTTATATGTTGCTCAGTCAGTACCCTTATTGGAAACCTGTGGTACGTTTA | | |
| | ACTTCAATACGCCGTCAGGATCTTTATTTTCAGAATTACAAAACATCATTCTCGCCCGGAAAAATCCGTTTTATATTGGCCACATACGGTCTCACTCTGGTCTTC | | |
| | CTGGACCTCTGGCAGAGGGTAATGATCGCATTGACAGAGCTCTAATAGGAGAAGCCTTAGTTTCAGATCGGGTTGCTTTGGCCCAACGTGATCTTGAAAGGT | | |
| | TTCATCTTTCTAGCCATACCCTAAGGCTCCGGCATAAGATCACAAAGGAGCAAGCGAGAATGATCCTAAAACAATGTCCTAAATGTATTACTTTATCTCCAGTG | | |
| | CCGCATCTAGGAGTTAATCCTAGAGGCCTTATGCCTAATCATATTTGGCAAATGGATATAACCCATTATGCAGAATTTGGAAAACTAAAATATATACATGTTTG | | |
| | CATTGATACTTGTTCAGGATTTCTTTTTGCTTCTCTGCATACAGGAGAAGCTTCAAAAAACGTAATTGATCATTGCCTACAAGCATTTAATGCCATGGGATTGCC | | |
| | TAAACTTATTAAGACAGACAATGGGCCATCTTATTCCAGTAAAAACTTTATTTCATTCTGTAAAGAATTCGGTATTAAACATAAAACTGGAATTCCTTACAACCC | | |
| | CATGGGACAGGGAATAGTTGAACGTGCTCATCGCACCTTAAAGAATTGGCTCTTTAAGACAAAAGAGGGGCAGCTATATCCCCCAAGGTCACCAAAGGCCCA | | |
| | CCTTGCCTTCACCTTATTTGTCCTAAATTTCTTGCACACCGATATCAAGGGCCAGTCTGCAGCGGATCGCCACTGGCATCCAGTTACTTCTAATTCTTATGCATT | | |
| | GGTAAAATGGAAGGACCCCCTGACTAATGAATGGAAGGGTCCAGATCCAGTTCTAATTTGGGGTAGGGGCTCAGTTTGTGTTTTTTCACGAGATGAAGATAG | | |
| | AGCGCGGTGGCTGCCAGAGAGATTAATTCGTCAGATGAACACAGATTCTGACTCTTCTGGTAAGTATCATTCTAAAGACTAAAATTCCTTTTGTGCTTAAAATT | | |
| | CAGCTGAGAGCAACAGCTCTCAAAGCTGTTCTCCAGCTACTCTCTGAGCCAGCTCCCGACAGGAGGCCGGAGACTAGCCTCAGCTTTACAATTTGCATTTGAA | | |
| | TAAAGTACCTAGACTTCCCCTAAAGAAGTTCTGTTTTCCTACTTTCTCACTGTCTTTCAAGATTTTGTCTTTCAAGCAGGTAAATCAACATTCTCGAAGCGGACC | | |
| | AGCGGATGTGCATCCCCGCCCCCCTAGAGCATGCAGGTGGCAGCTGTTATCCCCAGTCTCAGGACATTTCCAGCACATGGTTTTCAGTCTGAGTTAAAAATTT | | |
| | AGGTTTACCTAGAGGGCTAGAAGAGTAGATATTTCTATATTAATAAAGATTGGTTTTTATTTTGATAGACAGGCTTAGCCCCTTAGCTGACCTCTGGCTTTTCA | | |
| | CCCTTGCTGTTACTGCAAGGTGTCCTTAGCTCAATAGGCTGTGGAAAAAACAGGGATGAGGAGGAACGACTTCCAGCTCCTATTTTAGCCACAAATCGTGGT | | |
| | GTTACTAATGACATAATTCTTGCTTAGGCTTTGCTAATTCTGAGGTTGATAATTCTCCTTTAGGAGCTGCACAGCACTCAGAACTGTGCATACTGGTTTGTGATT | | |
| | GTACAAATTCAGTATGGGCATCGCTTGGTGCAGAGGTACTGCAAGGGAAGGTCCGGCTTGACCATTTCTGAGTTTCCTGTGAGATAAACCCGGTTTAAAAGA | | |
| | GGTTGGTATCATATTTTGGTTAAAAATAAAAAATATTTTCCGGCTCTACCTTGCCTCCCCAAAAGATACCCAGAGCCACATGTGTGGGTTTTACCAGTACCCAC | | |
| | GGGAGGAATCGGGTCCATGTCCACCCAAGCCAAGGTTAAAAGCCCACTCATCTACGGATGAGAAAATCATTTGATCACCTCAGTTAAGCGTTGCCTTATTTAA | | |
| | CTTAATTAATAGGGGGGAGAGAGATTGGAGACGTACTATTAAAAGGGCAAGCCCTTCACTGCCTCCCACCCAAATAAAAGAGCCGGTCGAACGCCTTCTCCC | | |
| | TGTTTCCCACCTATCATCCAAAAATGCGGAGGAATATCAACTTAGTGTTATTTTCCCATTTGTTCAGTCAAACTTAGCCAGAGTTCCAACGCCCTACTTAAAATT | | |
| | CAACTAGAAAGTTACCTACCAAGTACTAATTAGCATTATAAAGTCAGAGTCTGCAGCTCCAGGCCTTTCAGTTAGTTGTTTACTAGAAAGGACAGTCTTAAGCC | | |
| | AGATACAGTTTACCATAAGAAAAGTTAAAGAATCCCAGTGAAGCAAGTTTTTTCTTTAGCCCTAGATTCTAGGCAGAACTATTGAGCATAGATAATTTTTCCCC | | |
| | CCTCAGGCCAGCTTTTTCTTTTTTTTTAAATTTTGTTAATAAAAGGGAGGAGATGTAGTCTCCCCCTCCCCCAGCCTGAAACCTGCTTGCTCAGGGTGGAGCTTC | | |
| | CTGCTCATTCATTCTGCCACGCCCACTGCTGGAACCTGAGGAGCCACACACGTGCACCTTTCTACTGGACCCGAGATTATTCGGCGGGAATCGGGTCCCCTCC | | |
| | CCCTTCCTTCATAACTAGTGTCCCAACAATAAAATTTGAGCTTTGATCAGAATGAATTTGTCTTAGCTCCGTTTCTTCTTTCGCCCCGTCTAGATTCCTCTCTTAC | | |
| | AGCTCGAGTGGCCTTCTCAGTCGAACCGTTCACGTTGCGAGCTGCTGGCGGCCGCAACA | | |
| MusD4 | TGTAGTCTCCCCTCCCCTAGCCTGAAACCTGCTTGCTCGGGGTGGAGCTTCCTGCTCATTCGTTCTGCCATGCCCACTGCTGGAACCTGAGGAGCCACACACGT | 369 | 7454 |
| | GCACCTTTCTACTGAACCAGAGATTATTCGGCGGGAATCGGGTCCCCTCCCCCTTCCTTCATAACTAATGTCGCAACAATAAAATTTGAGCTTTGATCAGAATG | | |
| | AAATTGTCTTAGCTCCGTTTTTTCTTCCGCCCCGTCTAGATTCCTCTCTTACAGCTCGAGCGGCCTTCTCAGTCGAACCGTTCACGTTGCGAGCTGCTGGCGGCC | | |
| | GCAACATTTTGGCGCCCGAACAGGGACCTGAAGAATGGCAGAGAGATGCTAAGAGGAACGCTGCATTGGAGCTCCACAGGAAAGGATCTTCGTATCGGACA | | |
| | TCGGAGCAACGGACAGGTACACATGCTAGCGCTAGCTTAAAATTTCAGTTTTGTAAAGTGTTGCTGAGGATGCGGTAGGATACGAATTAAGCTTGAATCAGT | | |
| | GCTAACCCAACGCTGGTTCTGCTTGGGTCAGCAGCGTGTTAATCGGAACTAGAAACGGAAACAGGCAGGTTAGCCGCAGCTTTTTAGAAAGCTGCTTAGGTG | | |
| | GAAGAAGAAAGGGTTTAAAGTCATGGATCAGGCGGTTGCCCATAGTTTTCAGGAGTTGTTTCAGGCCAGAGGAGTAAGGCTTGAAGTACAATTAGTAAAAA | | |
| | ATTTTTTAGGTAAGATAGATAGCTGTTGCCCATGGTTCAAGGAAGAAGAAACACTAGATTGTGGAACCTGGGAGAAAGTTGGTGAGGCCTTAAAAATCACTC | | |
| | AGGCAGATAATTTTACCCTAGGCCTCTGGGCGCTCGTAAATGATGCAATAAAAGATGCCACTTCCCCAGGGCTAAGTTGCCCCCAGGCGGAGCTTGTGGTAT | | |
| | CTCAGGAGGAGTGCCTGTCAGAGAGGGCCTCCTCAGAAAAAGATCTTCTTAACTCAAAAATTGATAAATGTGGAAACTCGGATGAAAAACTGATTTTTAACA | | |
| | AAAATCACTCAGATAGAGGAGCTGCCCATTACCTTAATGAGAATTGGTCCTCTTGTGAATCTCCCGCTCCACCTATAGTCCCCACTTCGGGAGGTGCCACTCAT | | |
| | AGGGACACACGACTAAGCGAGTTAGAGTTTGAGATTAAGCTTCAGAGGCTGACTAATGAGCTTCGGGAACTAAAAAAGATGTCAGAAGCGGAGAAGAGTA | | |
| | ACTCTTCTGTAGTTCACCAGGTGCCGCTAGAAAAGGTTGTGAGTCAGGCTCGTGGGAAAGGACAGAATATGTCTAATACGCTAGCCTTTCCGGTGGTCGAGG | | |
| | TAGTTGATCAGCAAGATACTAGGGGCAGACATTACCAGACCTTAGATTTCAAGTTGATAAAAGAGTTAAAGGCGGCTGTTGTGCAATATGGCCCTTCAGCCC | | |
| | CATTCACTCAAGCATTACTGGACACAGTTGTGGAGTCACACTTAACCCCTTTAGATTGGAAGACTCTTTCTAAGGCTACCCTGTCAGAAGGAGATTTTTTGCTT | | |
| | TGGGATTCTGAATGGCGAGACGCCAGTAAGAAAACTGCTGCTTCTAACGCTCAGGCTGGTAATTCAGATTGGGATAGCAACATGCTTTTAGGAGAGGGCCCT | | |
| | TATGAGGGACAGACAAATCAGATTGATTTTCCCGTTGCAGTGTACGCGCAAATTGCGACGGCCGCACGCCGTGCTTGGGGAAGGTTGCCAGTCAAAGGAGA | | |
| | GATTGGTGGAAGTTTAGCTAGCATTCGGCAGAGTTCTGATGAACCATATCAGGATTTTGTGGACAGGCTATTGATTTCAGCTAGTAGAATCCTTGGGAATCCG | | |
| | GACACGGGAAGTCCTTTCGTTATGCAATTGGCTTATGAGAATGCTAATGCAATTTGCCGAGCTGCGATTCAACCGCATAAGGGAACGACAGATTTGGCAGGA | | |
| | TATGTCCGCCTTTGTGCAGACATCGGGCCTTCCTGCGAGACCTTGCAGGGAACCCACGCGCAGGCAATGTTCTCAAGGAAACGAGGGAAAAATGTATGCTTT | | |
| | AAGTGTGGAAGTTTAGATCATTTTAGAATTGATTGTCCTCAGAACAAGGGTGCCGAGGTTAGACAAACAGGCCGTGGCCCAGGAATATGTCCCCGGTGTGG | | |
| | GAAGGGCCGCCACTGGGCAAAAGATTGTAAGCATAAAACGAGGGTTTTGAGCCGCCCGGTGCCGGGAAACGAGGAAAGGGGTCAGCCCCAGGCCCCGAGT | | |
| | TACTCAAAGAAGACAGCTTATGGGGCTATAAATCTGCTGCCCAGCCAACAAGATCAGTTCTTGAGCTTGTCAGGTCAAACCCAGGAAATGCAAGACTGGACC | | |
| | TCTGTTCCACTGTCCATGCAGCATTAACCCCAGAAGTGGGAGTCCAAACTCTGCCTACCGGAGTTTTTGGACCACTACCTGTAGGAACCTGTGGTTTTCTCTTA | | |
| | GGACGAAGCAGTTCTATTGTAGAAGGCCTGCAGATTTATCCAGGTGTTATAAGTAATGATTATGAGGGAGAAATTAAAATCATAGCCGCTTGCCCTCGTGGT | | |
| | GCTATAACTATACCCGCTAATCAGAAAATTGCTCAACTTACTTTGATCCCTTTGCGCTGGTCACTATCTAAATTCCTTGAAAATGAAGAAGGACAGAATAACTT | | |
| | TGACTCCTCTGGTGTAAATTGGGTGAAATCTATCACCAATCAGAGACCTAACCTTAAATTGATTCTTGATGGAAAAAGCTTTGAAGGATTAATAGATACCGGG | | |
| | GCCGATGTAATGATCATCAGAGGGCAGGACTGGCCCTCAAACTGGCCCCTGTCTGTTTCCTTGACTCACCTTCAAGGAATTGGTTATGCCAGTAACCCAAAAC | | |
| | GTAGTTCCAAATTGCTAACCTGGAGAGATGAGGATGGAAAATCAGGAAATATTCAGCCGTATGTTATGCCAAATTTGCCTGTAACCCTGTGGGGAAGAGATC | | |
| | TGTTGTCACAGATGGGCGTTATCCTGTGCAGTTCTAAGGAGATGGTGACTGAACAGACGTTCAGGCAGGGACCCCTGCCTGATCGTGGACTAATAAAGAAG | | |
| | GGACAGAAAATTAAGACTTTTGAGGATCTTAAACCCCACTCTAACGTGAGAGGTTTAAAGTATTTTCAGTAGCGGCCGCTGTCTTGCCTGCATCCCACGCCGA | | |
| | AAAAATTCAATGGCGTAATGATATTCCGGTGTGGGTAGATCAGTGGTCTTTACCTAAAGAGAAAATAGAGGCCGCTTCTTTGCTAGTGCAGGAGCAGTTAGA | | |
| | AGCAGGACATTTGGTGGAGTCTCACTCTCCCTGGAATACACCCATTTTCATTATCAGGAAGAAATCGGGAAAATGGAGACTGTTGCAAGATTTAAGAAAGGT | | |
| | TAATGAAACCATGGTACTTATGGGAACTTTACAACCGGGGCTCCCCTCCCCAGTAGCCATTCCTAAGGGATATTATAAGATTGTTATAGATTTGAAAGATTGTT | | |
| | TCTTTACCATCCCTTTGCATCCAAAGGATTGTGAGAGATTTGCTTTTAGTGTTCCTTCTGTAAATTTCAAGGAACCCATGAAAAGATATCATTGGACAGTTCTCC | | |
| | CGCAGGGCATGGCTAATAGTCCCACCTTATGTCAAAGGTTTGTGGCAAAGGCAATTCAGCCTGTTAGACAACAATGGCCAAATATTTACATCATCCATTTCAC | | |
| | AGATGATGTCTTGATGGGGGAAAGGACCCCCAAGATTTGCTTTTGTGTTATGGAGACTTACGAAAGGCCCTGGCTGATAAGGGATTACAAATTGCTTCTGA | | |
| | AAAGATACAAACTCAGGATCCTTATAATTATTTGGGTTTTAGACTCACTGATCAAGATGTTTTTCCCCAGAAAATTGTTATTCGTAGAGATAACTTAAGGACCT | | |
| | TAAATGATTTTCAAAAATTGTTAGGTGATATAAATTGGCTTCGCCCCTATCTAAAGCTTACTACAGGGGAGTTGAAACCTTTATTTGATATTCTTAAAGGGAGT | | |
| | TCTGATCCCACTTCCCCTAGATCCCTAACCTCAGAAGGATTACTGGCCTTACAGCTAGTGGAAAAGGCTATTGAAGAACAGTTTGTCACTTACATAGATTACTC | | |
| | CCTGCCGCTGCACCTGTTAATTTTTAATACGACTCATGTGCCTACGGGATTGCTATGGCAAAAATTTCCTATAATGTGGATACATTCGAGGATTTCTCCCAAAC | | |
| | GTAATATCTTGCCATATCACGAAGCAGTGGCTCAGATGATTATCACTGGAAGAAGGCAGGCATTGACTTATTTTGGAAAGGAGCCAGATATCATTGTCCAGCC | | |
| | TTACAACGTGAGTCAGGACACTTGGCTGAAACAGCATAGTACAGATTGGTTGCTTGCGCAATTAGGGTTTGAAGGAACTCTAGATAGCCACTACCCCCAAGA | | |
| | TAGGTTGATAAAATTCTTAAATGTGCATGATATGATATTTCCTAAGATGACTTCCTTACAGCCTTTAAATAATGCTCTGTTGATTTTTACTGATGGCTCCTCTAA | | |
| | AGGGCGAGCTGGATATCTTATTAGTAATCAACAGGTTATCGTAGAGACTCCTGGTCTCTCGGCTCAGCTCGCCGAATTAACAGCAGTACTGAAGGTTTTTCAG | | |
| | TCTGTGCATGAGGCTTTTAATATTTTTACTGACAGTTTATATGTTGCTCAGTCAGTACCCTTATTGGAAACCTGTGGTACGTTTAACTTCAATACGCCGTCAGGA | | |
| | TCTTTATTTTCAGAATTACAAAACATCATTCTCGCCCGGAAAAATCCGTTTTATATTGGCCACATACGGTCTCACTCTGGTCTTCCTGGACCTCTGGCAGAGGGT | | |
| | AATGATCGCATTGACAGAGCTCTAATAGGAGAAGCCTTAGTTTCAGATCGGGTTGCTTTGGCCCAACGTGATCACGAAAGGTTTCATCTTTCTAGCCATACCC | | |
| | TAAGACTCCGACATAAGATCACAAAGGAGCAAGCGAGAATGATCGTAAAACAATGTCCTAAATGTATTACTTTATCTCCAGTGCCGCATCTAGGAGTTAATCC | | |
| | TAGACGCCTTATGCCTAATCATATTTGGCAAATGGATATAACCCATTATGCAGAATTTGGAAAACTAAAATATATACATGTTTGCATTGATACTTGTTCAGGAT | | |
| | TTCTTTTTGCTTCTCTGCATACAGGAGAAGCTTCAAAAAACGTAATTGATCATTGCCTACAAGCATTTAATGCCATGGGATTGCCTAAACGTATTAAGACAGAC | | |
| | AATGGGCCATCTTATTCCAGTAAAAACTTTATTTCATTCTGTAAAGAATTCGGTATTAAACATAAAACTGGAATTCCTTACAACCCCATGGGACAAGGAATAGT | | |
| | TGAACGTGCTCATCGCACCTTAAAGAATTGGCTTTTTAAGACAAAAGAGGGTCAGCTATATCCCCCAAGGTCACCAAAGGCCCACCTTGCCTTCACTTTATTTG | | |
| | TCCTAAATTTCTTGCACACTGATATCAAGGGCCAGTCTGCAGCGGATCGCCACTGGCATCCAGTTACTTCTAATTCTTATGCATTGGTAAAATGGAAGGACCCC | | |
| | CTGACTAATGAATGGAAGGGTCCAGATCCAGTTATAATTTGGGGTAGGGGCTCAGTTTGTGTTTTTTCACGAGATGAAGATGGAGCGTGGTGGCTGCCAGA | | |
| | GAGATTAATTCGTCAGATGAACACAGATTCTGACTCTTCTGGTAAGTATCATTCTAAAGACTAAAATTCCTTTTGTGCTTAAAATTCAGCTGAGAGCAACAGCT | | |
| | CTCAAAGCTGTTCTCCAGCTACTCTCTGAGCCAGCTTCCCGGCAGGAGGCCGGAGACTAGCCTCAGTTTTACAATTTGCATTTGAATAAAGTACCTAGACTTCC | | |
| | CTGAAAGAAGTTCTGCTTTCCTACTTTCTCACTGTCTTTCAAGATTTTGTCTTTGAAGCAGGTAAATCAACATTCCCGAGGCGGACCAGTGGATGTGCATCCCC | | |
| | GCCCCCCTAGAGCACACAGGTGGCAGCTGTTACCCCCAGTCTCAGGACATTTCCAGCACGTGGTTTTCAGTCTGAGTTAAAAATTTAGGTTTACCTAGAGGGC | | |
| | TAGAAGAGCAGATATTTCCATATTAATAAAGATTGGTTTTTATTTTGATAGACAGGCTTAGCCCCTTAGCTGACCTCTGGCTTTTCACCCTTGCTGTTACTGCAA | | |
| | GGTGTCCTTAGCTCAATAGGCTGTGGAAAAAACAGGGATGAGGAGGAACGACTTCCAGCTCCTATTTTAGCCACAAATCGTGGTGTTACTAACGACATAATT | | |
| | CTTGCTTAGGCTTTGCTAATTCTGAGGTTGATAATTCTCCTTTAGGAGCTGCACAGCACTCAGAACTGTGCATACTGGTTTGTGATTGTACAAATTCAGTATGG | | |
| | GCACCGCTTGGTGCAGAGATACTTACTGCAAGGGAAGGTCCGGCTTGACCATTTCTGAGTTTCCTGTGAGATAAACCCGGTTTGAAAGAGGTTGGTACCAAA | | |
| | TTTTGGTTAAAAATAAAAAATATTCTCCGGCTCTACCTCGCCTCCCCAAAAGGTACCGAGAGCCACATGTGTGGGTTTTACCCACGGGAGGAATCGGGTCCAT | | |
| | GTCCACCCAAGCCAAGGTTAAAAGCCCACTCATCTACGGATGAGAAAATCATTTGATCACCTCAGTTAAGCGTTGCCTTATTTAACTTAATTAATAGGGGGGA | | |
| | GAGAGATTGGAGACGTACTATTGAAAGGGCAAGCCCTTCACTGCCTCCCACCCAAATAAAAGAGCCGGTCGAACGCCTTCTCCCTGTTTCCCACCTATCTTCC | | |
| | AAAAATGCGGAGGAATTCAACTTAGTGTTATTTTCACATCGTTCAGTCAAACTTAGCCAGAGTTCCAACGCCCTACTTAAAATTCAACTAGAAAGTTACCTACC | | |
| | AAGTACTAATTAGCATTATAAAGTCAGAGTCTGCAGCTCCAGGCCTTTCAGTTAGTTGTTTACTAGAAAGGACAGTCTTAAGCCAGATACAGTTTACCATAAG | | |
| | AAAAGTTAAAGAATCCCAGTGAAGCAAGTTTTTTCTTTAGCCCTAGATTCCAGGCAGAACTATTGAGCATAGATAATTTTCCCCCCTCAGGCCAGCTTTTTCTTT | | |
| | TTTTTAATTTTGTTAATAAAAGGGAGGAGATGTAGTCTCCCCTCCCCTAGCCTGAAACCTGCTTGCTCGGGGTGGAGCTTCCTGCTCATTCGTTCTGCCACACC | | |
| | CACTGCTGGAACCTGAGGAGCCACACACGTGCACCTTTCTACTGGACCAGAGATTATTTGGCGGGAATCGGGTCCCCTCCCCCTTCCTTCATAACTAATGTCG | | |
| | CAACAATAAAATTTGGGCTTTGATCAGAATGAAATTGTCTTAGCTCCGTTTTTTCTTCCGCCCCGTCTAGATTCCTCTCTTACAGCTCGAGCGGCCTTCTCAGTC | | |
| | GAACCGTTCACGTTGCGAGCTGCTGGCGGCCGCAACA | | |
| MusD5 | TGTAGTCTCCCCTCCCCCAGCCTGAAACCTGCTTGCTCGGGGTGGAGCTTCCTGCTCATTCGTTCTGCCACGCCCACTGCTGGAACCTGAGGAGCCACACACGT | 370 | 7472 |
| | GCACCTTTCTACTGGACCCGAGATTATTCGGGGGGAATCGGGTCCCCTCCCCCTTCCTTCATAACTAGTGTCCCAACAATAAAATTTGAGCTTTGATCATAATG | | |
| | AATTTGTCTTAGCTCTGTTTCTTCTTTCGCCCCGTCTAGATTCCTCTCTTACAGCTCGAGTAGCCTTCTCAGTCGAACCGTTCATGTTGCGAGCTGCTGGCGGGT | | |
| | GCAACATTTTGGCGCCAGAACTGGGACCTGAAGAATGGCAGAGAGATGCTAAGAGGAACGCTGTGGTGGAGCTCCACAGGAAAGGATCTTCATATCGGAC | | |
| | ATCGGAGCAACGGACAGGTACACATGCTAGCGCTAGCTTAAAATTTCAGTTTTGTAAAGTGTTGCTGAGGATGCGGTAGGATACGAATTAAGCTTGAATCAG | | |
| | TGCTAACCCAACGCTGGTTCTGCTTGGGTCAGCAGCGTGTTAATCGGAACTAGAAACGGAAACAGGCAGGTTAGCCGCAGCTTTTTAGGAAGCTGCTTAGGT | | |
| | GGAAGAAGAAAGGGTTTAAAGTCATGGATCAGGCGGTTGCCCATAGTTTTCAGGAGTTGTTTCAGGCCAGAGGAGTAAGGCTTGAAGTACAATTAGTAAAA | | |
| | AAATTTTTAGGTAAGATAGATAGCTGTTGCCCATGGTTCAAGGAAGAAGAAACACTAAATTGTGGAACCTGGGAGAAAGTTGGTGAGGCCTTAAAAATCACT | | |
| | CAGGCAGATAATTTTACCCTAGGCCTCTGGGCGCTCGTAAATGATGCAATAAAAGATGCCACTTCCCCAGGGCTAAGTTGCCCCCAGGCGGAGCTTGTGGTA | | |
| | TCTCAGGAGGAGTGCCTGTCAGAGAGGGCCTCCTCAGAAAAAGATCTTCTTAACTCAAAAGTTGATAAATGTGGAAACTCAGATGAAAAACTGATTTTTAAC | | |
| | AAAAATCACTCAGATAGAGGAGCTGCCCATTACCTTAGTGAGAATTGGTCCTCTTGTGAATCTCCCGCTCCACCTGTAGTCCCCACTTCGGGAGGTGCCACTC | | |
| | ATAGGGACACACGACTAAGCGAGTTAGAGTTTGAGATTAAGCTTCAGAGGCTGACTAATGAGCTTCGGGAACTAAAAAAGATGTCAGAAGCGGAGAAGAG | | |
| | TAACTCTTCTGTAGTTCACCAGGTGCCGCTAGAAAAGGTTGTGAGTCAGGCTCGTGGGAAAGGACAGAATATGTCTAATACGCTAGCCTTTCCTGTGGTCGA | | |
| | GGTAGTTGATCAGCAAGATACTAGGGGCAGACATTGCCAGACCTTAGATTTCAAGTTGATAAAAGAGTTAAAGGCGGCTGTTGTGCAATATGGCCCTTCAGC | | |
| | CCCATTCACTCAAGCATTACTGGACACAGTTGTGGAGTCACACTTAACCCCTTTAGATTGGAAGACTCTTTCTAAGGCTACCCTGTCAGGAGGAGATTTTTTGC | | |
| | TTTGGGATTCTGAATGGCGAGACGCCAGTAAGAAAACTGCTGCTTCTAACGCTCAGGCTGGTAATTCAGGCTGGGATAGCAACATGCTATTAGGGGAGGGT | | |
| | CCATATGAAGGACAAACAAATCAGATTGATTTTCCTGTTGCAGTGTACGCACAAATTGCGACGGCCGCGCGCCGAGCTTGGGGAAGGTTGCCAGTCAAAGG | | |
| | AGAGATTGGTGGAAGTTTAGCTAGCATTCGGCAGAGTTCTGATGAACCATATCAGGATTTTGTGGACAGGCTATTGATTTCAGCTAGTAGAATCCTTGGGAA | | |
| | TCCGGACACGGGAAGTCCTTTCGTTATGCAATTGGCTTATGAGAATGCTAATGCAATTTGCCGAGCTGCGATTCAACCGCATAAGGGAACGACAGATTTGGC | | |
| | GGGATATGTCCGCCTTTGCGCAGACATCGGGCCTTCCTGCGAGATCTTGCAGGGAACCCACGTGCAAGCAATGTTCTCAAGGAAACGAGGGAAAAATGTAT | | |
| | GCTTTAAGTGTGGAAGTTTAGATCATTTTAGAATTGATTGTCCTCAGAACAAGGGTGCCGAGGTTAGACAAACAGGCCGTGCCCCAGGAATATGTCCCCGGT | | |
| | GTGGGAAGGACCGCCACTGGGCAAAAGATTGTAAGCATAAAACAAGGGTTTTGAGCCGCCCGGTGCCGGGAAATGAGGAAAGGGGTCAGACCCAGGCCCC | | |
| | GAGTTACTCAAAGAAGACAGCTTATGGGGCTATAAATCTGCTGCCCAGACAACAAGATCAGTTCTTGAGCTTGTCAGGTCAAACCCAGGAAATGCAAGACTG | | |
| | GACCTCTGTTCCACTGTCCATGCAGCATTAACCCCAGAAGTGGGAGTCCAAACTCTGCCTACCGGAGTCTTTGGACCACTACCTGTAGGAACCTGTGGTTTTCT | | |
| | CTTAGGACGAAGCAGTTCTATTGTAGAAGGCCTGCAGATTTATCCAGGTGTTATAAGTAATGATTATGAGGGAGAAATTAAAATCATAGCCGCTTGCCCTCGT | | |
| | GGTGCTCTAACTATACCCGCTAATCAGAAAATTGCTCAACTTACTTTGATCCCTTTGCGCTGGTCACTATCTAAATTCTTTGAAAATGAAGAAGGACAGAATAA | | |
| | CTTTGACTCCTCTGGCGTAAATTGGGTGAAATCTATCACCAATCAGAGACCTAACCTTAAATTGATTCTTGATGGAAAAAGCTTTGAAGGATTAATAGATACCG | | |
| | GGGCCGATGTAACGATTATTAGAGGGCAGGACTGGCCCTCAAACTGGCCCCTGTCTGTTTCCTTGACTCACCTTCAAGGAATTGGTTATGCCAGTAACCCAAA | | |
| | ACGTAGTTCCAAATTGCTAACCTGGAGAGATGAGGATGGAAAATCAGAAAATATTCAACCGTATGTTATGCCAAATTTGCCTGTAACCCTGTGGGGAAGAGA | | |
| | TCTGTTGTCACAGATGGGCGTTATCCTGTGCAGTTCTAAGGAGATGGTGACTGAACAGACGTTCAGGCAGGGACCCCTGCCTGATCGTGGACTAATAAAGAA | | |
| | GGGACAGAAAATTAAGACTTTTGAGGATCTTAAACCCCACTCTAACGTGAGAGGTTTAAAGTATTTTCAGTAGCGGCCACTGTCTTGCCTGCATCCCACGCCG | | |
| | AAAAAATTCAATGGCGTAATGATATTCCGGTGTGGGTAGATCAGTGGTCTTTACCTAAAGAGAAAATAGAGGCTGCTTCTTTGCTAGTGCAGGAGCAGTTAG | | |
| | AAGCAGGACATTTGGTGGAGTCTCACTCTCCCTGGAATACACCCATTTTCATTATCAGGAAGAAATCGGGAAAATGGAGACTGTTGCAAGATTTAAGAAAGG | | |
| | TTAATGAAACCATGGTACTTATGGGAACTTTACAACCGGGGCTCCCCTCCCCAGTAGCCATTCCTAAGGGATATTATAAGATTGTTATAGATTTGAAAGATTGT | | |
| | TTCTTTACCATCCCTTTGCATCCAAAGGATTGTGAGAGATTTGCTTTTAGTGTTCCTTCTGTAAATTTCAAGGAACCCATGAAAAGATATCAATGGACAGTTCTC | | |
| | CCGCAGGGCATGGCTAATAGTCCCACCTTATGTCAAAGGTTTGTGGCAAAGGCAATTCAGCCTGTTAGACAACAATGGCCAAATATTTACATCATTCATTTCAC | | |
| | AGATGATGTCTTGATGGCGGGAAAGGACCCCCAAGATTTGCTTTTGTGTTATGGAGACTTACGAAAGGCCCTGGCTGATAAGGGATTACAAATTGCTTCTGA | | |
| | AAAGATACAAACTCAGGATCCTTATAATTATTTGGGTTTTAGACTCACTGATCAAGCTGTTTTTCCCCAGAAAATTGTTATTCGTAGAGATAACTTAAGGACCT | | |
| | TAAATGATTTTCAAAAATTGTTAGGTGATATAAATTGGCTTCGCCCCTATCTAAAGCTTACTACAGGGGAGTTGAAACCTTTATTTGATATTCTTAAAGGGAGT | | |
| | TCTGATCCCACTTCCCCTAGATCCCTAACCTCAGAAGGACTACTGGCCTTACAGCTAGTGGAAAAGGCTATTGAAGAACAGTTCGTCACTTACATAGATTACTC | | |
| | CCTGCTGCTGCACCTGTTAATTTTTAATACGACTCATGTGCCTACAGGATTGCTATGGCAAAAATTTCCTATAATGTGGATACATTCGAGGATTTCTCCCAAAC | | |
| | GTAATATCTTGCCATATCACGAAGCAGTGGCTCAGATGATTATCACTGGAAGAAGGCAGGCATTGACTTATTTTGGAAAGGAGCCAGATATCATTGTCCAGCC | | |
| | TTACAGCGTGAGTCAGGACACTTGGCTGAAACAGCATAGTACAGATTGGTTGCTTGCACAATTAGGGTTTGAAGGAACTATAGATAGCCACTACCCCCAAGA | | |
| | TAGGTTGATAAAATTCTTAAATGTACATGATATGATATTTCCTAAGATGACTTTCTTACAGCCTTTAAATAATGCTCTATTGATTTTTACTGATGGCTCCTCTAAA | | |
| | GGGCGAGCTGGATATCTTATTAGTAATCAACAGGTTATTGTAGAGACTCCTGGTCTCTCGGCTCAGCTCGCCGAATTAACAGCAGTACTGAAGGTTTTTCAGT | | |
| | CTGTACATGAGGCTTTTAATATTTTTACTGACAGTTTATATGTTGCTCAGTCAGTACCCTTATTGGAAACCTGTGGTATGTTTAACTTCAATACGCTGTCAGGAT | | |
| | CTTTATTTTCAGAATTACAAAACATCATTCTCGCCCGGAAAAATCCGTTTTATATTGGCCACATACAGTCTCACTCTGGTCTTCCTGGACCTCTGGCAGAGGGTA | | |
| | ATGATCGCATTGACAGAGCTCTAATAGGAGAAGCCTTAGTTTCAGATCGGGTTGCTTTGGCCCAATGTGATCATGAAAGGTTTCATCTCTCTAGCCATACCCTA | | |
| | AGGCTCCGACATAAGATCACAAAGGAGCAAGCGAGAATGATTGTAAAACAATGTCCTAAATGTATTACTTTATCTCCAGTGCCGCATCTAGGAGTTAATCCTA | | |
| | GAGGCCTTATGCCTAATCATATTTGGCAAATGGATATAACCCATTATGCAGAATTTGGAAAACTAAAATATATACATGTTTGCATTGATACTTGTTCAGGATTT | | |
| | CTTTTTGCTTCTCTGCATACAGGAGAAGCTTCAAAAAACGTAATTGGTCATTGCCTACAAGCATTTAATGCCATGGGATTGCCTAAACTTATTAAGACAGACAA | | |
| | TGGGCCATCTTATTCCAGTAAAAACTTTATTTCATTCTGTAAAGAATTCGGTATTAAACATAAAACTGGAATTCCTTACAACCCCATGGGACAAGGAATAGTTG | | |
| | AACGTGCTCATCGCACCTTAAAGAATTGGCTTTTTAAAACAAAAGAGGGGCAGCTATATCCCCCAAGGTCACCAAAGGCCCACCTTGCCTTCACCTTATTTGTC | | |
| | CTAAATTTCTTGCACACCGATATCAAGGGCCAGTCTGCAGCGGATCGCCACTGGCATCCAGTTACTTCTAATTCTTATGCATTGGTAAAATGGAAGGACCCCCT | | |
| | GACTAATGAATGGAAGGGTCCAGATCCAGTTCTAATTTGGGGTAGGGGCTCAGTTTGTGTTTTTTCACGAGATGAAGATGGAGCGTGGTGGCTGCCAGAGA | | |
| | GATTAATTCGTCAGATGAACACAGATTCTGACTCTTCTGGTAAGTATCATTCGAAAGACTAAAATTCCTTTTGTGCTTAGAATTCAGCTGAGAGCTAAAGCTCC | | |
| | CAAAGCTGTTTTCCAGCTACTCTCTGAGCCAGCTCCCGACAGGAGGCCGGAGACTAGCCTCAGCTTTACAATTTGCATTTGAATAAAGTACCTAGACTTCCCTG | | |
| | AAAGAAGTTCTGCTTTCCTACTTTCTCACTGTCTTTCAAGATTTTGTCTTTCAAGCAGGTAAATCAACACTCTCGAAGCGGACCAGCGGATGTGCATCCCCGCC | | |
| | CCCCTAGAGCACACAGGTGGCTGCTGTTATCTTCTTTCCAAGGACATTTCCAGCACGTGGCTTTCAGTCTGAGTTAAAAATTAGGTTTACCTAGAGGGCTAGA | | |
| | AGAGTAGATATTTCTATATTAATAAAGATTGGTTTTTATTTTGATAGACAGGCTTAGCCCCTTAGCTGACCTCTGGCTTTTCACCCTTGCTGTTACTGCAAGGTG | | |
| | TCCTTAGGCTGTGGAAAAAACAGGGATGAGGAGGAACGACTTCCAGCTCCTATTTTAGCCACAAATCGTGGTGTTACTAACGACATAATTCTTGCTTAGGCTT | | |
| | TGCTAATTCTGAGGTTGATAATTCTCCTTTAGGAGCTGCACAGCACTCAGAACTGTGCATACTGGTTTGTGATTGTACAAATTCAGTATGGGCATCGCTTGGTG | | |
| | CAGAGGTACTGCAAGGGAAGGTCCGGCTTGACCATTTCTGAGTTTCCTGTGAGATAAACCCGGTTTAAAAGAGGTTGGTATCATATTTTGGTTAAAAATCAAA | | |
| | AATATTTTTCGGCTCTGCCTCGCCTCCCCAAAAGATACCCAGAGCCACATGTGTGGGTCTTACCAGTACCCACGGGGGGAATCGGGTCCATGTCCATCCAAGC | | |
| | CAAGGTTAAAAGCCCACTCATCTACGGATGAGAAAATCATTTGATCACCTCAGTTAAGCGTTGCCTTATTTAACTTAATTAATAGGGGGGAGAGAGATTGGAG | | |
| | ACGTACTATTGAAAGGGCAAGCCCTTCACTGCCTCCCACCCAAATAAAAAAGCCAATTGGCCTTGTACTACAAGAGCCGGTCGAACTCCTTCTCCCTGTTTCCC | | |
| | ACCTATCATCCAAAAATGCGGAGGAATATCAACTTAGTGTTATTTTCACATTGTTCAGTCAAACTTAGCCAGAGTTCCAACGCCCTACTTAAAATTCAACTAGA | | |
| | AAGTTACCTACCAAGTACTAATTAGCATTATAAAGTCAGAGTCTGCAGCTCCAGGCCTTTCAGTTAGTTGTTTACTAGAAAGGACAGTCTTAAGCCAGATACA | | |
| | GTTTACCATAAGAAAAGTTAAAGAATCCCAGTGAAGCAAGTTTTTTCTTTAGCCCTAGATTCCAGGCAGAACTATTGAGCATAGATAATTTTCCCCCCTCAGGC | | |
| | CAGCTTTTTCTTTTTTTTAAATTTTGTTAATAAAAGGGAGGAGATGTAGTCTCCCCTGCCCCAGCCTGAAACCTGCTTGCTCGGGGTGGAGCTTCCTGCTCATTC | | |
| | GTTCTGCCACGCCCACTGCTGGAACCTGAGGAGCCACACACGTGCACCTTTCTACTGGACCCGAGATTATTCGGCGGGAATCGAGTCCCCTCCCACTTCCTTC | | |
| | ATAACTAGTGTCCCAACAATAAAATTTGAGCTTTGATCAGAATGAATTTGTCTTAGCTCCGTTTCTTCTTTCGCCCCGTCTAGATTCCTCTCTTACAGCTCGAGTA | | |
| | GCCTTCTCAGTCGAACCATTCATGTTGCGAGCTGCTGGCGGCTGCAACA | | |
| MusD6 | TGTAGTCTCCCCTCCCCCAGCCTGAAACCTGCTTGCTCAGGGGTGGAGCTTCCCGCTCATCGCTCTGCCACGCCCACTGCTGGAACCTGCGGAGCCACACACG | 371 | 7492 |
| | TGCACCTTTCTACTGGACCAGAGATTATTCGGCGGGAATCGGGTCCCCTCCCCCTTCCTTCATAACTAGTGTCCCAACAATAAAATTTGAGCTTTGATCAGAAT | | |
| | GAATTTGTCTTGGCTCCGTTTCTTCTTTCGCCCCGTCTAGATTCCTCTCTTACAGCTCGAGTGGCCTTCTCAGTCGAACCGTTCACGTTGCGAGCTGCTGGCGGC | | |
| | CGCAACATTTTGGCGCCAGAACTGGGACCTGAAGAATGGCAGAGAGATGCTAAGAGGAACGCTGCTTTGGAGCTCCACAGGAAAGGATCTTCGTATCGGAC | | |
| | ATCGGAGCAACGGACAGGTACACATGCTAGCGCTAGCTTAAAATTTCAGTTTTGTAAAGTGTTGCTGAGGATGTGGTAGGATACGAATTAAGCTTGAATCAG | | |
| | TGCTAACCCAACGCTGGTTCTGCTTGGGTCAGCAGCGTGTTAATCGGAACTAGAAACGGAAACAGGCAGGTTAGCCGCAGCTTTTTAGGAAGCTGCTTAGGT | | |
| | GGAAGAAGAAAGGGTTTAAAGTCATGGATCAGGCGGTTGCCCATAGTTTTCAGGAGTTGTTTCAGGCCAGAGGAGTAAGGCTTGAAGTACAATTAGTAAAA | | |
| | AATTTTTTAGGTAAGATAGATAGCTGTTGCCCATGGTTCAAGGAAGAAGAAACACTAGATTGTGGAACCTGGGAGAAAGTTGGTGAGGCCTTAAAAATCACT | | |
| | CAGGCAGATAATTTTACCCTAGGCCTCTGGGCACTCATAAATGATGCAATAAAAGATGCCACTTCCCCAGGGCTAAGTTGCCCCCAGGCGGAGCTTGTGGTAT | | |
| | CTCAGGAGGAGTGCCTGTCAGAGAGGGCCTCCTCAGAAAAAGATCTTCTTAACTCAAAAATTGATAAATGTGGAAACTCGGATGAAAAACTGATTTTTAACA | | |
| | AAAATCACTCAGATAGAGGAGCTGCCCATTACCTTAATGAGAATTGGTCCTCTTGTGAATCTCCTGCTCAACCTGTAGTCCCCACTTCGGGAGGTGCCACTCAT | | |
| | AGGGACACACGACTAAGCGAGTTAGAGTTTGAGATTAAGCTTCAGAGGCTGACTAATGAGCTTCGGGAACTAAAAAAGATGTCAGAAGCGGAGAAGAGTA | | |
| | ACTCTTCTGTAGTTCACCAGGTGCCGCTAGAAAAGGTTGTGAGTCAGGCTCATGGGAAAGGACAGAATATCTCTAATACGCTAGCCTTTCCTGTGGTTGAGGT | | |
| | AGTTGATCAGCAAGATACTAGGGGCAGACATTACCAGACCTTAGATTTCAAGTTGATAAAAGAGTTAAAGGCGGCTGTTGTGCAATATGGCCCTTCAGCCCC | | |
| | ATTCACTCAAGCATTACTGGACACAGTTGTGGAGTCACACTTAACCCCTTTAGATTGGAAGACTCTTTCTAAGGCTACCCTGTCAGGAGGAGATTTTTTGCTTT | | |
| | GGGATTCTGAATGGCGAGACGCCAGTAAGAAAACTGCTGCTTCTAACGCTCAGGCTGGTAATTCAGACTGGGATAGCAACATGCTTTTAGGAGAGGGCCCTT | | |
| | ATGAGGGACAGACAAATCAGATTGATTTTCCCGTTGCAGTGTACGCGCAAATTGCGACGGCCGCACGCCGTGCTTGGGGAAGGTTGCCAGTCAAAGGAGAG | | |
| | ATTGGTGGAAGTTTAGCTAGCATTCGGCAGAGTTCTGATGAACCATATCAGGATTTTGTGGACAGGCTATTGATTTCAGCTAGTAGAATCCTTGGAAATCCGG | | |
| | ACACGGGAAGTCCTTTCGTTATGCAATTGGCTTATGAGAATGCTAACGCAATTTGCCGAGCTGCGATTCAACCGCATAAGGGAACGACAGATTTGGCGGGAT | | |
| | ATGTCCGTCTTTGCGCAGACATCGGGCCTTCCTGCGAGACCTTGCAGGGAACCCACGCGCAGGCAATGTTCTCTAGGAAACGAGGGAATAGTGCATGCTTTA | | |
| | AATGTGGAAGTTTAGATCATTTTAGAATTGATTGTCCTCAGAACAAGGGCGCCGAGGTTAGACAAACAGGCCGTGCCCCGGGAATATGTCCCCGATGTGGAA | | |
| | AGGGCCGCCACTGGGCGAAAGATTGCAAGCATAAAACGAGGGTTTTGAGCCGCCCGGTGCCGGGAAACGAGGAAAGGGGTCAGCCCCAGGCCCCAAGTTA | | |
| | CTCAAAGAAGACAGCTTATGGGGCTCTAAATCTGCTGCCCAGCCAACAAGATCAGTTCTTGAGCTTGTCAGGTCAAACCCAGGAAACGCAAGACTGGACCTC | | |
| | TGTTCCACTGTCCATGCAGCATTAACCCCAGAAGTGGGAGTCCAAACTCTGCCTACCGGAGTCTTTGGACCACTACCTGTAGGAACCTGTGGTTTTCTCTTAGG | | |
| | ACGAAGCAGTTCTATTGTAGAAGGCCTGCAGATTTATCCAGGTGTTATAAGTAATGATTATGAGGGAGAAATTAAAATCATAGCCGCTTGCCCTCGTGGTGCT | | |
| | ATAACTATACCCGCTAATCAGAAAATTGCTCAACTTACCTTGATCCCCTTGCGCTGGTCACTATCTAAATTCTCTAAAAATGAAGAAGGACAGATTAACTTTGA | | |
| | CTCCTCTGGCGTAAATTGGGTGAAATCTATCACTAATCAGAGACCTAACCTTAAATTGATTCTTGATGGAAAAAGCTTTGAAGGATTAATAGATACCGGGGCC | | |
| | GATGTAACCATTATTAGAGGGCAGGACTGGCCCTCAAACTGGCCCCTGTCTGTTTCCTTGACTCACCTTCAAGGAATTGGTTATGCCAGTAACCCAAAACGTA | | |
| | GTTCCAAATTGCTAACCTGGAGAGATGAGGATGGAAAATCAGGAAATATTCAGCCGTATGTTATGCAAAATTTGCCTGTAACCCTGTGGGGAAGAGATCTGT | | |
| | TGTCACAGATGGGCGTTATCCTGTGCAGTTCTAAGGAAATGGTGACTGAACAGACGTTCAGGCAGGGACCCCTGCCTGATCGTGGACTAATAAAGAAGGGA | | |
| | CAGAAAATTAAGACTTTTGAAGATCTTAAACCCCACTCTAACGTGAGAGGTTTAAAGTATTTTCAGTAGTGGCCGCTGTCTTGCCTGCATCCCACGCCGAAAA | | |
| | AATTCAATGGCGTAATGATATTCCGGTGTGGGTAGATCAGTGGTCTTTACCTAAAGAGAAAATAGAGGCCGCTTCTCTGCTAGTGCAGGAGCAGTTAGAAGC | | |
| | AGGACATTTGGTGGAGTCTCATTCTCCCTGGAATACACCCATTTTCATTATCAGGAAGAAATCGGGAAAATGGAGACTGTTGCAAGATTTAAGAAAGGTTAAT | | |
| | GAAACCATGGTACTTATGGGAACTTTACAACCGGGGCTCCCCTCCCCAGTAGCCATTCCTAAGGGATACTATAAGATTGTTATAGATTTGAAAGATTGTTTCTT | | |
| | TACCATCCCTTTGCATCCAGAGGATTGTGAGAGATTTGCTTTTAGTGTTCCTTCTGTAAATTTCAAGGAACCCATGAAAAGATATCAATGGACAGTTCTCCCGC | | |
| | AGGGGATGGCTAATAGTCCCACCTTATGTCAAAAGTTTGTGGCAAAGGCAATTCAGCCTGTTAGACAACAATGGCCAAATATTTACATCATTCATTTCACAGA | | |
| | TGATGTTTTGATGGCGGGAAAGGACCCCCAAGATTTGCTTTTGTGTTATGGAGACTTACGAAAGGCCCTGGCTGATAAGGGATTACAAATTGCTTCTGAAAA | | |
| | GATACAAACTCAGGATCCTTATAATTATTTGGGTTTTAGACTCACTGACCAAGCTGTTTTTCACCAGAAAATTGTTATTCGTAGAGATAACTTAAGGACCTTAA | | |
| | ATGATTTTCAAAAATTGTTAGGTGATATAAACTGGCTTCGCCCCTATCTAAAGCTTACTACAGGGGAGTTGAAACCTTTATTTGATATTCTTAAAGGGAGTTCT | | |
| | GATCCTACTTCCCCTAGATCCCTAACCTCAGAAGGTTTACTGGCCTTACAGCTAGTGGAAAAGGCTATTGAAGAACAGTTTGTCACTTACATAGATTACTCCCT | | |
| | GCCGCTGCACCTGTTAATTTTTAACACGACTCATGTGCCTACGGGATTGCTATGGCAAAAATTTCCTATAATGTGGATACATTCAAGGATTTCTCCCAAACGTA | | |
| | ATATTTTGCCATATCATGAAGCAGTGGCTCAGATGATTATCACTGGAAGAAGGCAGGCATTGACTTATTTTGGAAAGGAGCCAGATATCATTGTCCAGCCTTA | | |
| | CAGCGTGAGTCAGGACACTTGGCTGAAACAGCATAGTACAGATTGGTTGCTTGCACAATTAGGGTTTGAAGGAACTATAGATAGCCACTACCCCCAAGATAG | | |
| | GTTGATAAAATTCTTAAATGTACATGATATGATATTTCCTAAGATGACTTCCTTACAGCCTTTAAATAATGCTCTATTGATTTTTACTGATGGCTCCTCTAAAGG | | |
| | GCGAGCTGGATATCTTATTAGTAATCAACAGGTTATCGTAGAGACTCCTGGTCTCTCGGCTCAGCTCGCCGAACTAACAGCAGTACTGAAGGTTTTTCAGTCT | | |
| | GTACAGGAGGCTTTTAATATTTTTACTGACAGTTTATATGTTGCTCAGTCAGTACCCTTATTGGAAACCTGTGGTACTTTTAACTTCAATACGCCGTCAGGATCT | | |
| | TTATTTTCAGAATTACAAAACATCATTCTCGCCCGGAAAAATCCGTTTTATATTGGCCACATACGGTCTCACTCTGGTCTTCCTGGACCTCTGGCAGAGGGTAA | | |
| | TAATTGCATTGACAGAGCTCTAATAGGAGAAGCCTTAGTTTCAGATCGGGTTGCTTTGGCCCAACGTGATCATGAAAGGTTTCATCTCTCTAGCCATACCCTAA | | |
| | GGCTCCGACATAAGATCACCAAGGAGCAAGCGAGAATGATTGTAAAACAATGTCCTAAATGTATTACTTTATCTCCAGTGCCGCATCTAGGAGTTAATCCTAG | | |
| | AGGCCTTATGCCTAATCATATTTGGCAAATGGATATAACCCATTATGCAGAATTTGGAAAACTAAAATATATACATGTTTGCATTGATACTTGTTCAGGATTTC | | |
| | TCTTTGCTTCTCTGCATACAGGAGAAGCTTCAAAAAACGTAATTGATCATTGCCTACAAGCATTTAATGCCATGGGATTACCTAAACTTATTAAGACAGACAAT | | |
| | GGGCCATCTTATTCCAGTAAAAACTTTATTTCATTCTGTAAAGAATTCGGTATTAAACATAAAACTGGAATTCCTTACAACCCCATGGGACAAGGAATAGTTGA | | |
| | ACGTGCTCATCGCACCTTAAAGAATTGGCTCTTTAAGACAAAAGAGGGGCAGCTATATCCCCCAAGGTCTCCAAAGGCCCACCTTGCCTTCACCTTATTTGTCC | | |
| | TAAATTTCTTGCACACCGATATCAAGGGCCAGTCTGCAGCGGATCGCCACTGGCATCCAGTTACTTCTAATTCTTATGCATTGGTAAAATGGAAGGACCCCCT | | |
| | GACTAATGAATGGAAGGGTCCAGATCCAGTTCTAATTTGGGGTAGAGGCTCAGTTTGTGTTTTTTCACGAGATGAAGATGGAGCACGGTGGCTGCCAGAGA | | |
| | GATTAATTCGTCAGACGAACACAGATTCTGACTCTTCTGGTAAGTATCATTCTAAAGACTAAAATTCCTTTTGTGCTTAAAATTCAGCTGAGAGCAACAGCTCT | | |
| | CAAAGCTGTTCTCCAGCTACTCTCTGAGCCAGCTCCCGACAGGAGGCCGGAGACTAGCCTCAGCTTTACAATTTGCATTTAAATAAAGTACCTAGACTTCCCCG | | |
| | AAAAAAGTTCTGCTTTTCTACTTTCTCACTGTCTTTCAAGATTTTGTCTTTCAAGCAGGTAAATCAACATTCTCGAGGCGGACCAGCGGATGTGCATCCCCGCCC | | |
| | CCCTAGAGCACTCAGGTGGCAGCTGTTATCCCCAGTCTCAGGACATTCCAGCATGTGGCCTTCAGTCTGAGTTAAAAATTAGGTTTACCCAGAGGACTAGAAT | | |
| | AGTAGATATTTCTATATTAATAAAGATTGGTTTTTATTTTGATAGACAGGCTTAGCCCCTTAGCTGACCTCTGGCTTTTCACCCTTGCTGTTACTGCAAGGTGTC | | |
| | TTTAGCTCAATAAGGCTGTGGAAAAAAACAGGGATGAGGAGGAACGGCTCCCAGCTCCTATTTTAGCCACAAATCGTGGTGTTACTAACGACATAATTCTTGC | | |
| | TTAGGCTTTGCTAAATCTGAGGTTGATAATTCTCCTTTAGGAGCTGCACAGCGCTCAGAACTGTGCATACTGATTTGTGATGGTACAAATTCAGTATGGGCATC | | |
| | GCTTGGTGCAGATGGAGGTACTGCAAGGAAAGGTCCCAGCTTGACCATTTCTGAGTTTCCTGTGAGATAAACCCGGTTTGAAAGAGGTTGGTACCAAATTAT | | |
| | ATATCCCTCGGCTCTACCTCGCCTCCCCAAAAGGTACCAGAGCCACAGGTGTGGATTTTAACAGAATCCACGGGAGGAATCGGGTCCATGTCCACCCAAGCCA | | |
| | AGGTTAAAAGCCCACTCATCTACGGATGAGAAAATCATTTGATCACCTCAGTTAAGCGCTGCCTTATTTTAACTTAATTAATAGGGGGGAGAGAGATTGGAGA | | |
| | CTTACTATTGAAAGGGCAAGCCCTTCACTGCCTCCCACCCAAATAAAAAAGCCAATTGGCCTTGTACTACAGAGCTGGCCGGACCCCTTATCCCTGTTACCCAC | | |
| | CAATCATCCAAAAATGCGGAGGAATATCAACTTAGTGTTATTCTTATTATAGTGTATTTCACACTTGTTCAGTCAAACTTAGCCAGAGTTCCAACGCCCTACTTA | | |
| | AAATTCAACTAGAAAGTTACCTACCAAGTACTAATTAGCATTATAAAGTCAGAGCCTACAGCTCCAGGCTTTTCAGTTAGTTGTTTACTAAGATAAGAAAAGAC | | |
| | AGTCTTAGCCAGATACAGTTTACCATAATAAAAGTTAAAGAATCCCAGGGAAGCAAGTTTTTTCTTTTAGCCCTAGATTCCAGGCAGAACTATTGAGCATAGA | | |
| | TAATTTTTCCCCCTCAGGCCAGCTTTTTCTTTTTTTTTAATTTTGTTAATAAAAGGGAGGAGATGTAGTCTCCCCTCCCCCAGCCTGAAACCTGCTTGCTCAGGG | | |
| | GTGGAGCTTCCCGCTCATCGCTCTGCCACGCCCACTGCTGGAACCTGCGGAGCCACACACGTGCACCTTTCTACTGGACCAGAGATTATTCGGCGGGAATCG | | |
| | GGTCCCCTCCCCCTTCCTTCATAACTAGTGTCCCAACAATAAAATTTGAGCTTTGATCAGAATGAATTTGTCTTGGCTCCGTTTCTTCTTTCGCCCCGTCTAGATT | | |
| | CCTCTCTTACAGCTCGAGTGGCCTTCTCAGTCGAACCGTTCACGTTGCGAGCTGCTGGCGGCCGCAACA | | |
| MusD7 | TGTAGTCTCCCCTCCCCCAGCCTGAAACCTGCTTGCTCGGGGTGGAGCTTCCTGCTCATTCGTTCTGCCACGCCCACTGCTGGAACCTGAGGAGCCACACACGT | 372 | 7450 |
| | GCACCTTTCTACTGGACCCGAGATTATTCGGCGGGAATCGGGTCCCCTCCCCCTTCCTTCATAACTAGTGTCCCAACAATAAAATTTGAGCTTTGATCAGAATG | | |
| | AATTTGTCTTAGCTCCGTTTCTTCTTTCGCCCCGTCTAGATTCCTCTCTTACAGCTCGAGTGGCCTTCTCAGTCGAACCGTTCACGTTGCGAGCTGCTGGCGGCC | | |
| | GCAACATTTTGGCGCCAGAACTGGGACCTGAAGAATGGCAGAGAGATGCTAAGAGGAACGCTGCATTGGAGCTCCACAGGAAAGGATCTTCGTATCGGACA | | |
| | TCGGAGCAACGGACAGGTACACATGCTAGCGCTAGCTTAAAATTTCAGTTTTGTAAAGTGATGCTGAGGATGCGGTAGGATACGAATTAAGCTTGAATCAGT | | |
| | GCTAACCCAACGCTGGTTCTGCTTGGGTCAGCAGCGTGTTAATCGGAACTAGAAACAGAAACAGGCAGGTTAGCCGCAGCTTTTTAGGAAGCTGCTTAGGTG | | |
| | GAAGAAGAAAGGGTTTAAAGTCATGGATCAGGCGGTTGCCCATAGTTTTCAGGAGTTGTTTCAGGCCAGAGGAGTAAGGCTTGAAGTACAATTAGTAAAAA | | |
| | AATTTTTAGGTAAGATAGATAGCTGTTGCCCATGGTTCAAGGAAGAAGAAACACTAGATTGTGGAACCTGGGAGAAAGTTGGTGAGGCCTTAAAAATCACTC | | |
| | AGGCAGATAATTTTACCCTAGGCCTCTGGGCGCTCGTAAATGATGCAATAAAAGATGCCACTTCCCCAGGGCTAAGTTGCCCCCAGGCGGAGCTTGTGGTAT | | |
| | CTCAGGAGGAGTGCCTGTCAGAGAGGGCCTCCTCAGAAAAAGATCTTCTTAACTCAAAAATTGATAAATGTGGAAACTCGGATGAAAAACTGATTTTTAACA | | |
| | AAAATCACTCAGATAGAGGAGCTGCCCATTACCTTAATGAGAATTGGTCCTCTTGTGAATCTCCCGCTCCACCTGTAGTCCCCACTTCGGGAGGTGCCACTCAT | | |
| | AGGGACACACGACTAAGCGAGTTAGAGTTTGAGATTAAGCTTCAGAGGCTGACTAATGAGCTTCGGGAACTAAAAAAGATGTCAGAAGCGGAGAAGAGTA | | |
| | ACTCTTCTGTAGTTCACCAGGTGCCGCTAGAAAAGGTTGTGAGTCAGGCTCGTGGGAAAGGACAGAATATGTCTAATACGCTAGCCTTTCCGGTGGTCGAGG | | |
| | TAGTTGATCAGCAAGATACTAGGGGCAGACATTACCAGACCTTAGATTTCAAGTTGATAAAAGAGTTAAAGGCGGCTGTTGTGCAATATGGCCCTTCAGCCC | | |
| | CATTCACTCAAGCATTACTGGACACAGTTGTGGAGTCACACTTAACCCCTTTAGATTGGAAGACTCTTTCTAAGGCTACCCTGTCAGGAGGAGATTTTTTGCTT | | |
| | TGGGATTCTGAATGGCGAGACGCCAGTAAGAAAACTGCTGCTTCTAACGCTCAGGGTGGTAATTCAGACTGGGATAGCAACATGCTTTTAGGAGAGGGCCC | | |
| | TTATGAGGGACAGATAAATCAGATTGATTTTCCCGTTGCAGTGTACGCGCAAATTGCGACGGCCGCACGCCTTGCTTGGGGAAGATTGCCAGTCAAAGGAGA | | |
| | GATTGGTGGAAGTTTAGCTAGCATTCGGCAGAGTTCTGATGAACCATATCAGGATTTTGTGGACAGGCTATTGATTTCAGCTAGTAGAATCCTTGGGAATCCG | | |
| | GACACGGGAAGTCCTTTCGTTATGCAATTGGCTTATGAGAATGCTAATGCAATTTGCCGAGCTGCGATTCAACCGCATAAGGGAACGACAGATTTGGCGGGA | | |
| | TATGTCCGCCTTTGCGCAGACATCGGGCCTTCCTGCGAGACCTTGCCGGGAACCCACGCGCAGGCAATGTTCTCAAGGAAACGAGGGAAAAATGTATGCTTT | | |
| | AAGTGTGGAAGTTTAGATCATTTTAGAATTGATTGTCCTCAGAACAAGGGTGCCGAGGTTAGACAAACAGGCCGTGCCCCAGGAATATGTCCCCGGTGTGGG | | |
| | AAGGGCCGCCACTGGGCAAAAGATTGTAAGCATAAAACGAGGGTTTTGAGCCGCCCAGTGCCGGGAAACGAGGAAAGGGGTCAGCCCCAGGCCCCGAGTT | | |
| | ACTCAAAGAAGACAGCTTATGGGGCTATAAATCTGCTGCCCAGCCAACAAGATCAGTTCTTGAGCTTGTCAGGTCAAACCCAGGAAATGCAAGACTGGACCT | | |
| | CTGTTCCACTGTCCATGCAGCATTAACCCCAGAAGTGGGAGTCCAAACTCTGCCTACCGGAGTCTTTGGACCACTACCTGTAGGAACCTGTGGTTTTCTCTTAG | | |
| | GACGAAGCAGTTTTATTGTAGAAGGCCTGCAGCTTTATCCAGGTGTTATAAGTAATGATTATGAGGGAGAAATTAAAATCATAGCCGCTTGCCCTCGTGGTGC | | |
| | TATAACTATACCCGCTAATCAGAAAATTGCTCAACTTACTTTGATCCCTTTGCGCTGGTCACTATCTAAATTCTTTGAAAATGAAGAAGGACAGAATAACTTTG | | |
| | ACTCCTCTGGCGTAAATTGGGTGAAATCTATCACCAATCAGAGACCTAACCTTAAATTGATTCTTGATGGAAAAAGCTTTGAAGGATTAATAGATACCGGGGC | | |
| | CGATGTAACGATTATTAGAGGGCAGGACTGGCCCTCAAACTGGCCCCTGTCTGTTTCCTTGACTCACCTTCAAGGAATTGGTTATGCCAGTAACCCAAAACGT | | |
| | AGTTCCAAATTGCTAACCTGGAGAGATGAGGATGGAAAATCAGGAAATATTCAGCCGTATGTTATGCCAAATTTGCCTGTGACCCTGTGGGGAAGAGATCTG | | |
| | TTGTCACAGATGGGCGTTATCCTGTGCAGTTCTAAGGAGATGGTGACTGAACAGACGTTCAGGCAGGGACCCCTGCCTGATCGTGGACTAATAAAGAAGGG | | |
| | ACAGAAAATTAAGACTTTTGAGGATCTTAAACCCCACTCTAACGTGAGAGGTTTAAAGTATTTTCAGTAGCGGCCACTGTCTTGCCTGCATCCCACGCCGAAA | | |
| | AAATTCAATGGCGTAATGATATTCCGGTGTGGGTAGATCAGTGGTCTTTACCTAAAGAGAAAATAGAGGCCGCTTCTTTGCTAGTGCAGGAGCAGTTAGAAG | | |
| | CAGGACATTTGGTGGAGTCTCACTCTCCCTGGAATACACCCATTTTCATTATCAGGAAGAAATCGGGAAAATGGAGACTGTTGCAAGATTTAAGAAAGGTTA | | |
| | ATGAAACCATGGTACTTATGGGAACTTTACAACCGGGGCTCCCCTCCCCAGTAGCCATTCCTAAGGGATATTATAAGATTGTTATAGATTTGAAAGATTGTTTC | | |
| | TTTACCATCCCTTTGCATCCAAAGGATTGTGAGAGATTTGCTTTTAGTGTTCCTTCTGTAAATTTCAAGGAACCCATGAAAAGATATCAATGGACAGTTCTCCC | | |
| | GCAGGGCATGGCTAATAGTCCCACCTTATGTCAAAGGTTTGTGGCAAAGGCAATTCAGCCTGTTAGACAACAATGGCCAAATATTTACATCATTCATTTCACA | | |
| | GATGATGTCTTGATGGCGGGAAAGGACCCCCAAGATTTGCTTTTGTGTTATGGAGACTTACGAAAGGCCCTGGCTGATAAGGGATTACAAATTGCTTCTGAA | | |
| | AAGATACAAACTCAGGATCCTTATAATTATTTGGGTTTTAGACTCACTGATCAAGCTGTTTTTCCCCAGAAAATTGTTATTTGTAGAGATAACTTAAGGACCTTA | | |
| | AATGATTTTCAAAAATTGTTAGGTGATATAAATTGGCTTCGCCCCTATGTAAAGCTTACTACAGGGGAGTTGAAACCTTTATTTGATATTCTTAAAGGGAGTTC | | |
| | TGATCCCACTTCCCCTAGATCCCTAACCTCAGAAGGATTACTGGCCTTACAGCTAGTGGAAAAGGCTATTGAAGAACAGTTTGTCACTTACATAGATTACTCCC | | |
| | TGCCGCTGCACCTGTTAATTTTTAATACGACTCATGTGCCTACGGGATTGCTATGGCAAAAATTTCCTATAATGTGGATACATTCGAGGATTTCTCCCAAACGT | | |
| | AATATCTTGCCATATCATGAAGCAGTGGCTCAGATGATTATCACTGGAAGAAAGCAGGCATTGACTTATTTTGGAAAGGAGCCAGATATCATTGTCCAGCCTT | | |
| | ACAGCGTGAGTCAGGACACTTGGCTGAAACAGCATAGTACAGATTGGTTGCTTGCACAATTAGGGTTTGAAGGAACTGTAGATAGCCACTACCCCCAAGATA | | |
| | GGTTGATAAAATTCTTAAATGTACATGATATGATATTTCCTAAGATGACTTCCTTACAGCCTTTAAATAATGCTCTATTGATTTTTACTGATGGCTCCTCTAAAG | | |
| | GGCGAGCTGGATATCTTATTAGTAATCAACAGGTTATCGTAGAGACTCCTGGTCTCTCGGCTCAGCTCGCCGAATTAACAGCAGTACTGAAGGTTTTTCAGTC | | |
| | TGTACATGAGGCTTTTAATATTTTTACTGACAGTTTATATGTCGCTCAGTCAGTACCCTTATTGGAAACCTGTGGTACGTTTAACTTCAATACGCCGTCAGGATC | | |
| | TTTATTTTCAGAATTACAAAACATCATTCTCGCCCGGAAAAATCCGTTTTATATTGGCCACATACGGTCTCACTCTGGTCTTCCTGGACCTCTGGCAGAGGGTA | | |
| | ATGATCGCATTGACAGAGCTCTAATAGGAGAAGCCTTAGTTTCAGATCGGGTTGCTTTGGCCCAACGTGATCACAAAAGGTTTCATCTTTCTAGCCATACCCTA | | |
| | AGGCTCCGACATAAGATCACAAAGGAGCAAGCGAGAATGATCGTAAAACAATGTCCTAAAAGTATTACTTTATCTCCAGTGCTGCATCTAGGAGTTAATCCTA | | |
| | GAGGCCTTATGCCTAATCATATTTGGCAAATGGATATAACCCATTATGCAGAATTTGGAAAACTAAAATATATACATGTTTGCATTGATACTTGTTCAGGATTT | | |
| | CTTTTTGCTTCTCTGCATACAGGAGAAGCTTCAAAAAACGTAATTGATCATTGCCTACAAGCATTTAATGCCATGGGATTGCCTAAACTTATTAAGACAGACAA | | |
| | TGGGCCATCTTATTCCAGTAAAAACTTTATTTCATTCTGTAAAGAATTCGGTATTAAACATAAAACTGGAATTCCTTACAACCCCATGGGACAAGGAATAGTTG | | |
| | AACGTGCTCATCGCACCTTAAAGAATTGGCTTTTTAAGACAAAAGAGGGGCAGCTATATCCCCCAAGGTCACCAAAGGCCCACCTTGCCTTCACTTTATTTGTC | | |
| | CTAAATTTCTTGCACACCGATATCAAGGGCCAGTCTGCAGCGGATCGCCACTGGCATCCAGTTACTTCAAATTCTTATGCATTGGTAAAATGGAAGGACCCCC | | |
| | TGACTAATGAATGGAAGGGTCCAGATCCAGTTCTAATTTGGGGTAGGGGCTCAGTTTGTGTTTTTTCACGAGATGAAGATGGAGCGCGATGGCTGCCAGAG | | |
| | AGATTAATTCGTCAGATGAACACAGATTCTGACTCTTCTGGTAAGTATCATTCTAAAGACTAAAATTCCTTTTGTGCTTAAAATTCAGCTGAGAGCAACAGCTC | | |
| | TCAAAGCTGTTCTCCAGCTACTCTCTGAGCCAGCTCCCGACAGGAGGCCGGAGACTAGCCTCAGCTTTACAATTTGCATTTGAATAAAGTACCTAGACTTCCCC | | |
| | GAAAGAAGTTCTGCTTTCCTACTTTCTCGCTGTCTTTCAAGATTTTGTCTTTCAAGCAGGTAAATCAACATTCTCGAGGCAGACCAGCGGATGTGCATCCCCGC | | |
| | CCCCCTAGAGCACACAGGTGGCAGCTGTTATCCCCAGTCTCAGGACATTTCCAGCACGTGGTTTTCAGTCTGAGTTAAAAATTTAGGTTTACCTAGAGGGCTA | | |
| | GAAGAGTAGATATTTCTATATTAATAAAGATTGGTTTTTATTTTGATAGACAGGCTTAGCCCCTTAGCTGACCTCTGGCTTTTCACCCTTGCTGTTACTGCAAGG | | |
| | TGTCCTTAGCTCAATAGGCTGTGGAAAAAACAGGGATGAGGAGGAACGACTTCCAGCTCCTATTTTAGCCACAAATCGTGGTGTTACTAACGACATAATTCTT | | |
| | GCTTAGGCTTTGCTAATTCTGAGGTTGATAATTCTCCTTTAGGAGCTGCACAGCACTCAGAACTGTGCATACTGGTTTGTGATTGTACAAATTCAGTATGGGCA | | |
| | CCGCTTGGTGCAGAGGTACTGCAAGGGAAGGTCCGGCTTGACCATTTCTGAGTTTCCTGTGAGATAAACCCGGTTTAAAAGAGGTTGGTATCATATTTTGGTT | | |
| | AAAAATAAAAAATATTTTCCGGCTCTACCTCACCTCCCCAAAAGGTACCGAGAGCCACATGTGTGGGTTTTACCCACGGGAGGAATCGGGTCCATGTCCACCC | | |
| | AAGCCAAGGTTAAAAGCCCACTCATCTACGGATGAGAAAATCATTTGATCACCTCAGTTAAGCATTGCCTTATTTAACTTAATTAATAGGGGGGAGAGAGATT | | |
| | GGAGACGTACTATTGAAAGGGCAAGCCCTTCACTGCCTCCCACCCAAATAAAAGAGCCGGTCGAACTCCTTCTCCCTGTTTCCCACCTATCATCCAAAAATGCG | | |
| | GAGGAATATCAACTTAGTGTTATTTTCACATTGTTCAGTCAAACTTAGCCAGAGTTCCAACGCCCTACTTAAAATTCAACTAGAAAGTTACCTACCAAGTACTA | | |
| | ATTAGCATTATAAAGTCAGAGTCTGCAGCTCCAGGCCTTTCAGTTAGTTGTTTACTAGAAAGGACAGTCTTAAGCCAGATACAGTTTACAATAAGAAAAGTTA | | |
| | AAGAATCCCAGTGAAGCAAGTTTTTTCTTTAGTCCTAGATTCCAGGCAGAACTATTGAGCATAGATAATTTTCCCCCCTCAGGCCAGCTTTTTCTTTTTTTAAAT | | |
| | TTTGTTAATAAAAGGGAGGAGATGTAGTCTCCCCTCCCCCAGCCTGAAACCTGCTTGCTCGGGGTGGAGCTTCCTGCTCATTCGTTCTGCCACGCCCACTGCTG | | |
| | GAACCTGAGGAGCCACACACGTGCACCTTTCTACTGGACCCGAGATTATTCGGCGGGAATCGGGTCCCCTCCCCCTTCCTTCATAACTAGTGTCCCAACAATA | | |
| | AAATTTGAGCTTTGATCAGAATGAATTTGTCTTAGCTCCGTTTCTTCTTTCGCCCCGTCTAGATTTCTCTCTTACAGCTCGAGTGGCCTTCTCAGTCGAACCGTTC | | |
| | ACGTTGCGAGCTGCTGGCGGCCGCAACA | | |
| MusD8 | TGTAGTCTCCCCTCCCCTAGCCTGAAACCTGCTTGCTCGGGGTGGAGCTTCCTGCTCATTCGTTCTGCCACGCCCACTGCTGGAACCTGAGGAGCCACACACGT | 373 | 7456 |
| | GCACCTTTCTACTGGACCAGAGATTATTCGGCGGGAATCGGGTCCCCTCCCCCTTCCTTCATAACTAGTGTCGCAACAATAAAATTGGAGCTTTGATCAGAATG | | |
| | AAATTGTCTTAGCTCCGTTTTTTCTTCCGCCCTGTCTAGATTCCTCTCTTACAGCTCGAGCGGCCTTCTCAGTCGAACCGTTCACGTTGCGAGCTGCTGGCAGCC | | |
| | GCAACATTTTGGCGCCAGAACTGGGACCTGAAGAATGGCAGAGAGATGCTAAGAGGAACGCTGCATTGGAGCTCCACAGGAAAGGATCTTCGTATCGGACA | | |
| | TCGGAGCAACGGACAGGTACACATGCTAGCGCTAGCTTAAAATTTCAGTTTTGTAAAGTGTTGCTGAGGATGCGGTAGGATACGAATTAAGCTTGAATCAGT | | |
| | GCTAACCCAACGCTGGTTCTGCTTGGGTCAGCAGCGTGTTAATCGGAACTAGAAACGGAAACAGGCAGGTTAGCCGCAGCTTTTTAGGAAGCTGCTTAGGTG | | |
| | GAAGAAGAAAGGGTTTAAAGTCATGGATCAGGCGGTTGCTCATAGTTTTCAGGAGTTGTTTCAGGCCAGAGGAGTAAGGCTTGAAGTACAATTAGTAAAAA | | |
| | ATTTTTTAGGTAAGATAGATAGCTGTTGCCCATGGTTCAAGGAAGAAGAAACACTAGATTGTGGAACCTGGGAGAAAGTTGGTGAGGCCTTAAAAATCACTC | | |
| | AGGCAGATAATTTTACCCTAGGCCTCTGGGCGCTCGTAAATGATGCAATAAAAGATGCCACTTCCCCAGGGCTAAGTTGCCCCCAGGCGGAGCTTGTGGTAT | | |
| | CTCAGGAGGAGTGCCTGTCAGAGAGGGCCTCCTCAGAAAAAGATCTTCTTAACTCAAAAATTGATAAATGTGGAAACTCGGATGAAAAACTGATTTTTAACA | | |
| | AAAATCACTCAGATAGAGGAGCTGCCCATTACCTTAATGAGAATTGGTCCTCTTGTGAATCTCCCGCTCCACCTGTAGTCCCCACTTCGGGAGGTGCCACTCAT | | |
| | AGGGACACACGACTAAGCGAGTTAGAGTTTGAGATTAAGCTTCAGAGGCTGACTAATGAGCTTCGGGAACTAAAAAAGATGTCAGAAGCAGAGAAGAGTA | | |
| | ACTCTTCTGTAGTTCACCAGGTGCCGCTAGAAAAGGTTGTGAGTCAGGCTCGTGGGAAAGGACAGAATATGTCTAATACGCTAGCCTTTCCGGTGGTCGAGG | | |
| | TAGTTGATCAGCAAGATACTAGGGGCAGACATTACCAGACCTTAGATTTCAAGTTGATAAAAGAGTTAAAGGCGGCTGTTGTGCAATATGGCCCTTCAGCCC | | |
| | CATTCACTCAAGCATTACTGGACACAGTTGTGGAGTCACACTTAACCCCTTTAGATTGGAAGGCTCTTTCTAAGGCTACCCTGTCAGGAGGAGATTTTTTGCTT | | |
| | TGGGATTCTGAATGGCGAGACGCCAGTAAGAAAACTGCTGCTTCTAACGCTCAGGCTGGTAATTCAGACTGGGATAGCAACATGCTTTTAGGAGAGGGCCCT | | |
| | TATGAGGGACAGACAAATCAGATTGATTTTCCCGTTGCAGTGTACGCGCAAATTGCGACGGCCGCACGCCGTGCTTGGGGAAGGTTGCCAGTCAAAGGAGA | | |
| | GATTGGTGGAAGTTTAGCTAGCATTCGGCAGAGTTCTGATGAACCATATCAGGATTTTGTGGACAGGCTATTGATTTCAGCTAGTAGAATCCTTGGGAATCCG | | |
| | GACATGGGAAGTCCTTTCGTTATGCAATTGGCTTATGAGAATGCTAATGCAATTTGCCGAGCTGCGATTCAACCGCATAAGGGAACGACAGATTTGGCGGGA | | |
| | TATGTCCGCCTTTGCGCAGACATCGGGCCTTCCTGCGAGACCTTGCAGGGAACCCACGCGCAGGCAATGTTCTCAAGGAAACGAGGGAAAAATGTATGCTTT | | |
| | AAGTGTGGAAGTTTAGATCATTTTAGAATTGATTGTCCTCAGAACAAGGGTGCCGAGGTTAGACAAACAGGCCGTGGCCCAGGAATATGTCCCCGATGTGGA | | |
| | AAGGGCCGCCACTGGGCAAAAGATTGTAAGCATAAAACGAGGGTTTTGAGCCGCCCGGTGCCGGGAAACGAGGAAAGGGGTCAGCCCCAGGCCCCGAGTT | | |
| | ACTCAAAGAAGACAGCTTATGGGGCTATAAATCTGCTGCCCAGCCAACAAGATCAGTTCTTGAGCTTGTCAGGTCAAACCCAGGAAATGCAAGACTGGACCT | | |
| | CTGTTCCACTGTCCATGCAGCATTAACCCCAGAAGTGGGAGTCCAAACTCTGCCTACCGGAGTTTTTGGACCACTACCTGTAGGAACCTGTGGTTTTCTCTTAG | | |
| | GACGAAGCAGTTCTATTGTAGAAGGCCTGCAGATTTATCCAGGTGTTATAAGTAATGATTATGAGGGAGAAATTAAAATCATAGCCGCTTGCCCTCGTGGTG | | |
| | CTATAACTATACCCGCTAATCAGAAAATTGCTCAACTTACTTTGATCCCTTTGCGCTGGTCACTATCTAAATTCTTTGAAAATGAAGAAAGACAGAATAACTTTG | | |
| | ACTCCTCTGGCGTAAATTGGGTGAAATCTATCACCAATCAGAGACCTAACCTTAAATTGATTCTTGATGGAAAAAGCTTTGAAGGATTAATAGATACCGGGGC | | |
| | CGATGTAACGATTATTAGAGGGCAGGACTGGCCCTCAAACTGGCCCCTGTCTGTTTCCTTGACTCACCTTCAAGGAATTGGTTATGCCAGTAACCCAAAACGT | | |
| | AGTTCCAAATTGCTAACCTGGAGAGATGAGGATGGAAAATCAGGAAATATTCAGCCGTATGTTATGCCAAATTTGCCTGTAACCCTGTGGGGAAGAGATCTG | | |
| | TTGTCACAGATGGGCGTTATCCTGTGCAGTTCTAAGGAGATGGTGACTAAACAGACGTTCAGGCAGGGACCCCTGCCTGATCGTGGACTAATAAAGAATGGA | | |
| | CAGAAAATTAAGACTTTTGAGGATCTTAAACCCCACTCTAACGTGAGAGGTTTAAAGTATTTTCAGTAGCGGCCACTGTCTTGCCTGCATCCCACGCCGAAAA | | |
| | AATTCAATGGCGTAATGATATTCCGGTGTGGGTAGATCAGTGGTCTTTACCTAAAGAGAAAATAGAGGCCGCTTCTTTGCTAGTGCAGGAGCAGTTAGAAGC | | |
| | AGGACATTTGGTGGAGTCTCACTCTCCCTGGAATACACCCATTTTCATTATCAGGAAGAAATCGGGAAAATGGAGACTGTTGCAAGATTTAAGAAAGGTTAAT | | |
| | GAAACCATGGTACTTATGGGAACTTTACAACCGGGGCTCCCCTCCCCAGTAGCCATTCCTAAGGGATATTATAAGATTGTTATAGATTTGAAAGATTGTTTCTT | | |
| | TACCATCCCTTTGCATCCAAAGGATTGTGAGAGATTTGCTTTTAGTGTTCCTTCTGTAAATTTCAAGGAACCCATGAAAAGATATCATTGGACAGTTCTCCCGC | | |
| | AGGGCATGGCTAATAGTCCCACCTTATGTCAAAGGTTTGTGGCAAAGGCAATTCAGCCTGTTAGACAACAATGGCCAAATATTTACATCATCCATTTCACAGA | | |
| | TGATGTCTTGATGGCGGGAAAGGACCCCCAAGATTTGCTTTTGTGTTATGGAGACTTACGAAAGGCCCTGGCTGATAAGGGATTACAAATTGCTTCTGAAAA | | |
| | GATACAAACTCAGGATCCTTATAATTATTTGGGTTTTAGACTCACTGATCAAGCTGTTTTTCCCCAGAAAATTGTTATTCGTAGAGATAACTTAAGGACCTTAA | | |
| | ATGATTTTCAAAAATTGTTAGGTGATATAAATTGGCTTCGCCCCTATCTAAAGCTTACTACAGGGGAGTTGAAACCTTTATTTGATATTCGTAAAGGGAGTTCT | | |
| | GATCCCACTTCCCCTAGATCCCTAACCTCAGAAGGATTACTGGCCTTACAGCTAGTGGAAAAGGCTATTGAAGAACAGTTTGTCACTTACATAGATTACTCCCT | | |
| | GCCGCTGCACCTGTTAATTTTTAATACGACTCATGTGCCTACGGGATTGCTATGGCAAAAATTTCCTATAATGTGGATACATTCGAGGATTTCTCCCAAACGTA | | |
| | ATATCTTGCCATATCACGAAGCAGTGGCTCAGATGATTATCACTGGAAGAAGGCAGGCATTGACTTATTTTGGAAAGGAGCCAGATATCATTGTCCAGCCTTA | | |
| | CAGCGTGAGTCAGGACACTTGGCTGAAACAGCATAGTACAGATTGGTTGCTTGCACAATTAGGGTTTGAAGGAACTCTAGATAGCCACTACCCCCAAGATAG | | |
| | GTTGATAAAATTCTTAAATGTGCATGATATGATATTTCCTAAGATGACTTCCTTACAGCCTTTAAATAATGCTCTATTGATTTTTACTGATGGCTCCTCTAAAGG | | |
| | GCGAGCTGGATATCTTATTAGTAATCAACAGGTTATCGTAGAGACTCCTGGTCTCTCGGCTCAGCTCGCTGAATTAACAGCAGTACTGAAGGTTTTTCAGTCT | | |
| | GTGCATGAGGCTTTTAATATTTTTACTGACAGTTTATATGTTGCTCAGTCAGTACCCTTATTGGAAACCTGTGGTACGTTTAACTTCAATACGCCGTCAGGATCT | | |
| | TTATTTTCAGAATTACAAAACATCATTCTCGCCCGGAAAAATCCGTTTTATATTGGCCACATACGGTCTCACTCTGGTCTTCCTGGACCTCTGGCAGAGGGTAA | | |
| | TGATCGCATTGACAGAGCTCTAATAGGAGAAGCCTTAGTTTCAGATCGGGTTGCTTTGGCCCAACGTGATCACGAAAGGTTTCATCTTTCTAGCCATACCCTA | | |
| | AGGCTCCGACATAAGATCACAAAGGAGCAAGCGAGAATGATCGTAAAACAATGTCCTAAATGTATTACTTTATCTCCAGTGCCGCATCTAGGAGTTAATCCTA | | |
| | GAGGCCTTATGCCTAATCATATTTGGCAAATGGATATAACCCATTATGCAGAATTTGGAAAACTAAAATATATACATGTTTGCATTGATACTTGTTCAGGATTT | | |
| | CTTTTTGCTTCTCTGCATACAGGAGAAGCTTCAAAAAACGTAATTGATCATTGCCTACAAGCATTTAATGCCATGGGATTGCCTAAACTTATTAAGACAGACAA | | |
| | TGGGCCATCTTATTCCAGTAAAAACTTTATTTCATTCTGTAAAGAATTCGGTATTAAACATAAAACTGGAATTCCTTACAACCCCATGGGACAAGGAATAGTTG | | |
| | AACGTGCTCATCGCACCTTAAAGAATTGGCTTTTTAAGACAAAAGAGGGTCAGCTATATCCCCCAAGGTCACCAAAGGCCCACCTTGCCTTCACTTTATTTGTC | | |
| | CTAAATTTCTTGCACACCGATATCAAGGGCCAGTCTGCAGCGGATCGCCACTGGCATCCAGTTACTTCTAATTCTTATGCATTGGTAAAATGGAAGGACCCCCT | | |
| | GACTAATGAATGGAAGGGTCCAGATCCAGTTCTAATTTGGGGTAGGGGCTCAGTTTGTGTTTTTTCACGAGATGAAGATGGAACGCGGTGGCTGCCAGAGA | | |
| | GATTAATTCGTCAGATGAACACAGATTCTGACTCTTCTGGTAAGTATCATTCTAAAGACTAAAATTCCTTTTGTGCTTAAAATTCAGCTGAGAGCAACAGCTCT | | |
| | CAAAGCTGTTCTCCAGCTACTCTCTGAGCCAGCTCCCGACAGGAGGCCGGAGACTAGCCGCAGCTTTACAATTTGCATTTGAATAAAGTACCTAGACTTCCCC | | |
| | GAAAGAAGTTCTGCTTTCCTACTTTCTCACTGTCTTTCAAGATTTTGTCTTTCAAGCAGGTAAATCAACATTCTCGAGGTGGACCAGTGGATGTGCATCCCCGC | | |
| | CCCCCTAGAGCACACAGGTGGCAGCTGTTACCCCCAGTCTCAGGACATTTCCAGCACGTGGTTTTCAGTCTGAGTTAAAAATTTAGGTTTACCTAGAGGGCTA | | |
| | GAAGAGTAGATATTTCTGTATTTATAAAGATTGGTTTTTATTTTGATAGACAGGCTTAGCCCCTTAGCTGACCTCTGGCTTTTCACCCTTGCTGTTACTGCAAGG | | |
| | TGTCCTTAGCTCAATAGGCTGTGGAAAAAACAGGGATGAGGAGGAACGACTTCCAGCTCCTATTTTAGCCACAAATCGTGGTGTTACTAACGACATAATTCTT | | |
| | GCTTAGGCTTTGCTAATTCTGAGGTTGATAATTCTCCTTTAGGAGCTGCACAGCACTCAGAACTGTGCATACTGGTTTGTGATTGTACAAATTCAGTATGGGCA | | |
| | CCGCTTGGTGCAGAGATACTTACTGCAAGGGAAGGTCCGGCTTGACCATTTCTGAGTTTCCTGTGAGATAAACCCGGTTTGAAAGAGGTTGGTACCAAATTTT | | |
| | GGTTAAAAATAAAAAATATTTTCCGGCTCTACCTCGCCTCCCCAAAAGGTAGCGAGAGCCACATGTGTGGGTTTTACCCACAGGAGGAATCGGGTCCATGTCC | | |
| | ACCCAAGCCAAGGTTAAAAGCCCACTCATCTACGGATGAGAAAATCATTTGATCACCTCAGTTAAGCGTTGCCTTATTTAACTTAATTAATAGGGGGGGAGAG | | |
| | AGATTGGAGACGTACTATTGAAAGGGCAAGCCCTTTACTGCCTCCCACCCAAATAAAAGAGCCGGTCGAACGCCTTCTCCCTGTTTCCCACCTATCTTCCAAAA | | |
| | ATGCGGAGGAATATCAACTTAGTGTTATTTTCACATCGTTCAGTCAAACTTAGCCAGAGTTCCAACGCCCTACTTAAAATTCAACTAGAAAGTTACCTACCAAG | | |
| | TACTAATTAGCATTATAAAGTCAGAGTCTGCAGCTCCAGGCCTTTCAGTTAGTTGTTTACTAGAAAGGACAGTCTTAAACCAGATACAGTTTACCATAAGAAAA | | |
| | GTTAAAGAATCCCAGTGAAGCAAGTTTTTTCTTTAGCCCTAGATTCCAGGCAGAACTATTGAGCATAGATAATTTTCCCCCCTCAGGCCAGCTTTTTCTTTTTTT | | |
| | TTAATTTTGTTAATAAAAGGGAGGAGATGTAGTCTCCCCTCCCCTAGCCTGAAACCTGCTTGCTCGGGGTGGAGCTTCCTGCTCATTCGTTCTGCCACGCCCAC | | |
| | TGCTGGAACCTGAGGAGCCACACACGTGCACCTTTCTACTGGACCAGAGATTATTCGGCGGGAATCGGGTCCCCTCCCCCTTCCTTCATAACTAGTGTCGCAA | | |
| | CAATAAAATTGGAGCTTTGATCAGAATGACATTGTCTTAGCTCCGTTTTTTCTTCCGCCCCGTCTAGATTCCTCTCTTACAGCTCCAGCGGCCTTCTCAGTCGAA | | |
| | CCGTTCACGTTGCGAGCTGCTGGCGGCCGCAACA | | |
| MusD9 | TGTAGTCTCCCCTCCCCCAGCCTGAAACCTGCTTGCTCGGGGTGGAGCTTCCTGCTCATTCGTTCTGCCACGCCCACTGCTGGAACCTGAGGAGCCACACACGT | 374 | 7403 |
| | GCACCTTTCTACTGGACCCGAGATTATTCGGCGGGAATCGGGTCCCCTCCCCCTTCCTTCATAACTAGTGTCGCAACAATAAAATTTGAGCTTTGATCAGTATG | | |
| | AATTTGTCTTAGCTCCGTTTCTTCTTTTGCCCCGTCTAGATTCCTCTCTTACAGCTCGAGCGGCCTTCTCAGTCGAACCGTTCACATTGCGAGCTGCTGGCGGCC | | |
| | GCAACATTTTGGCGCCAGAACTGGGACCTGAAGAATGGCAGAGAGATGCTAAGAGGAACGCTGCATTGGAGCTCCACAGGAAAGGATCTTCGTATCGGACA | | |
| | TCGGAGCAACGGACAGGTACACATGCTAGCGCTAGCTTAAAATTTCAGTTTTGTAAAGTGTTGCTGAGGATGCGGTAGGATACGAATTAAGCTTGAATCAGT | | |
| | GCTAACCCAACGCTGGTTCTGCTTGGGTCAGCAGCGTGTTAATCGGAACTAGAAACGGAAACAGGCAGGTTAGCCGCAGCTTTTTAGGAAGCTGCTTAGGTG | | |
| | GAAGAAGAAAGGGTTTAAAGTCATGGATCAGGCGGTTGCCCATAGTTTTCAGGAGTTGTTTCAGGCCAGAGGAGTAAGGCTTGAAGTACAATTAGTAAAAA | | |
| | AATTTTTAAGTAAGATAGATAGCTGTTGCCCATGGTTCAAGGAAGAAGAAACACTAGATTGTGGAACCTGGGAGAAAGTTGGTGAGGCCTTAAAAATCACTC | | |
| | AGGCAGATAATTTTACCCTAGGCCTCTGGGCGCTCGTAAATGATGCAATAAAAGATGCCACTTCCCCAGGGCTAAGTTGCCCCCAGGCGGAGCTTGTGGTAT | | |
| | CTCAGGAGGAGTGCCTGTCAGAGAGGGCCTCCTCAGAAAAAGATCTTCTTAACTCAAAAATTGATAAATGTGGAAACTCGGATGAAAAACTGATTTTTAACA | | |
| | AAAATCACTCAGATAGAGGAGCTGCCCATTACCTTAATGAGAATTGGTCCTCTTGTGAATCTCCCGCTCCACCTGTAGTCCCCACTTCGGGAGGTGCCACTCAT | | |
| | AGGGACACACGACTAAGCGAGTTAGAGTTTGAGATTAAGCTTCAGAGGCTGACTAATGAGCTTCGGGAACTAAAAAAGATGTCAGAAGCGGAGAAGAGTA | | |
| | ACTCTTCTGTAGTTCACCAGGTGCCGCTAGAAAAGGTTGTGAGTCAGGCTCGTGGGAAAGGACAGAATATGTCTAATACGCTAGCCTTTCCTGTGGTCGAGG | | |
| | TAGTTGATCAGCAAGATACTAGGGGCAGACATTACCAGACCTTAGATTTCAAGTTGATAAAAGAGTTAAAGGCGGCTGTTGTGCAATATGGCCCTTCAGCCC | | |
| | CATTCACTCAAGCATTACTGGACACAGTTGTGGAGTCACACTTAACCCCTTTAGATTGGAGGACTCTTTCTAAGGCTACCCTGTCAGGAGGAGATTTTTTGCTT | | |
| | TGGGATTCTGAATGGCGAGACGCCAGTAAGAAAACTGCTGCTTCTAACGCTCAGGCTGGTAATTCAGACTGGGATAGCAACATGCTTTTAGGAGAGGGCCCT | | |
| | TATGAGGGACAGACAAACCAGATTGATTTTCCCGTTGCAGTGTACGCGCAAATTGCGACGGCCGCACGCCGTGCTTGGGGAAGGTTGCCTGTCAAAGGAGA | | |
| | GATTGGTGGAAGTTTAGCTAGCATTCGGCAGAGTTCTGATGAACCATATCAGGATTTTGTGGACAGGCTATTGATTTCAGCTAGTAGAATCCTTGGAAATCCG | | |
| | GACACGGGAAGTCCTTTCGTTATGCAATTGGCTTATGAGAATGCTAATGCAATTTGCCGAGCTGCGATTCAACCGCATAAGGGAACGACAGATTTGGCGGGA | | |
| | TATGTCCGCCTTTGCACAGACATCGGGCCTTCCTGCGAGACCTTGCAGGGAACCCACGCGCAGGCAATGTTCTCAAGGAAACGAGGGAAAAATGTATGCTTT | | |
| | AAGTGTGGAAGTTTAGATCATTTTAGAATTGATTGTCCTCAGAACAAGGGTGCCGAGGTTAGACAAACAGGCCGTGCCCCAGGAATATGTCCCCGGTGTGGG | | |
| | AAGGGCCGCCACTGGGCAAAAGATTGTAAGCATAAAACGAGGGTTTTGAGCCGCCCGGTGCCGGGAAACGAGGAAAGGGGTCAGCCCCAGGCCCCAAGTT | | |
| | ACTCAAAGAAGACAGCTTATGGGGCTATAAATCTGCTGCCCAGCCAACAAGATCAGTTCTTGAGCTTGTCAGGTCAAACCCAGGAAATGCAAGACTGGACCT | | |
| | CTGTTCCACTGTCCATGCAGCATTAATCCCAGAAGTGGGAGTCCAAACTCTGCCTACCGGAGTCTTTGGACCACTACCTGTAGGAACCTGTGGTTTTCTCTTAG | | |
| | GACGAAGCAGTTCTATTGTAGAAGGCCTGCAGATTTATCCAGGTGTTATAAGTAATGATTATGAGGGAGAAATTAAAATCATAGCCGCTTGCCCTCGTGGTG | | |
| | CTATAACTATACCCGCTAATCAGAAAATTGCTCAACTTACTTTGATCCCTTTGCGCTGGTCACTATCTAAATTCTTTGAAAATGAAGAAGGACAGAATAACTTTG | | |
| | ACTCCCTTGGCGTAAATTGGGTGAAATCTATCACTAATCAGAGACCTAACCTTAAATTGATTCTTGATGGAAAAAGCTTTGAAGGATTAATAGATACCGGGGC | | |
| | CAATGTAACGATAATTAGAGGGCAGGACTGGCCCTCAAACTGGCCCCTGTCTGTTTCCTTGACTCACCTTCAAGGAATTGGTTATGCCAGTAACCCAAAACGT | | |
| | AGTTCCAAATTGCTAACCTGGAGAGATGAGGATGGAAAATCAGGAAATATTCAGCCGTATGTTATGCCAAATTTGCCTGTAACCCTGTGGGGAAGAGATCTG | | |
| | TTGTCACAGATGGGCGTTATCCTGTGCAGTTCTAAGGAGATGGTGACTGAACAGACGTTCAGGCAGGGACCCCTGCCTGATCGTGGACTAATAAAGAAGGG | | |
| | ACAGAAAATTAAGACTTTTGAGGATCTTAAACCCCACTCTAACGTGAGAGGTTTAAAGTATTTTCAGTAGCGGCCACTGTCTTGCCTGCATCCCACGCCGAAA | | |
| | AAATTCAATGGCGTAATGATATTCCGGTATGGGTAGATCAGTGGTCTTTACCTAAAGAGAAAATAGAGGCCGCTTCTTTGCTAGTGCAGGAGCAGTTAGAAG | | |
| | CAGGACATTTGGTGGAGTCTCACTCTCCCTGGAATACACCCATTTTCATTATCAGGAAGAAATCGGGAAAATGGAGACTGTTGCAAGATTTAAGAAAGGTTA | | |
| | ATGAAACCATGGTACTTATGGGAACTTTACAACCGGGGCTTCCCTCCCCAGTAGCCATTCCTAAGGGATATTATAAGATTGTTATAGATTTGAAAGATTGTTTC | | |
| | TTTACCATCCCTTTGCATCCAAAGGATTGTGAGAGATTTGCTTTTAGTGTTCCTTCTGTAAATTTCAAGGAACCCATGAAAAGATATCAATGGACAGTTCTCCC | | |
| | GCAGGGCATGGCTAATAGTCCCACCTTATGTCAAAGGTTTGTGGCAAAGGCAATTCAGCCTGTTAGACAACAATGGCCAAATATTTACATCATTCATTTCACA | | |
| | GATGATGTCTTGATGGCGGGAAAGGACCCCCAAGATTTGCTTTTGTGTTATGGAGACTTACAAAAGGCCCTGGCTGATAAGGGATTACAAATTGCTTCTGAA | | |
| | AAGATACAAACTCAGGATCCTTATAATTATTTGGGTTTTAGACTCACTGATCAAGCTGTTTTTCCCCAGAAAATTGTTATTCGTAGAGATAACTTAAGGACCTT | | |
| | AAATGATTTTCAAAAATTGTTAGGTGATATAAATTGGCTTCGCCCCTATCTAAAGCTTACTACAGGGGAGTTGAAACCTTTATTTGATATTCTTAAAGGGAGTT | | |
| | CTGATCCCACTTCCCCTAGATCCCTAACCTCAGAAGGATTACTGGCCTTACAGCTAGTGGAAAAGGCTATTGAAGAACAGTTTGTCAATTACATAGATTACTCC | | |
| | CTGCCGCTGCACCTGTTAATTTTTAATACGACTCATGTGCCTACGGGATTGCTATGGCAAAAATTTCCTATAATGTGGATACATTCGCGGATTTCTCCCAAACG | | |
| | TAATATCTTGCCATATCACGAAGCAGTGGCTCAGATGATTATCACTGGAAGAAGGCAGGCATTGACTTATTTTGGAAAGGAGCCAGATATCATTGTCCAGCCT | | |
| | TACAGCGTGAGTCAGGACACTTGGCTGAAACAGCATAGTACAGATTGGTTGCTTCCACAATTAGGGTTTGAAGGAACTCTAGATAGCCACTACCCCCAAGAT | | |
| | AGGTTGATAAAATTCTTAAATGTGCATGATATGATATTTCCTAAGATGACTTCCTTACAGCCTTTAAATAATGCTCTATTGATTTTTACTGATGGCTCCTCTAAA | | |
| | GGGCGAGCTGGATATCTTATTAGTAATCAACAGGTTATCATAGAGACTCCTGGTCTCTCGGCTCAGCTCGCCGAATTAACAGCAGTACTGAAGGTTTTTCAGT | | |
| | CTGTACATGAGGCTTTTAATATTTTTACTGACAGTTTATATGTTGCTCAGTCAGTACCCTTATTGGAAACCTGTGGTACGTTTAACTTCAATACGCCGTCAGGAT | | |
| | CTTTATTTTCAGAATTACAAAACATCATTCTCGCCCGGAAAAATCCGTTTTATATTGGCCACATACGGTCTCACTCTGGTCTTCCTGGACCTCTGGCAGAGGGTA | | |
| | ATGATCGCATTGACAGAGCTCTAATAGGAGAAGCCTTAGTTTCAGATCGGGTTGCTTTGGCCCAACGTGATCACGAAAGGTTTCATCTTTCTAGCCATACCCT | | |
| | AAGGCTCCGACATAAGATCACAAAGGGGCAAGCGAGAATGATCGTAAAACAATGTCCTAAATGTATTACTTTATCTCCAGTGCCGCATCTAGGAGTTAATCCT | | |
| | AGAGGCCTTATGCCTAATCATATTTGGCAAATGGATATAACCCATTATGCAGAATTTGGAAAACTAAAATATATACATGTTTGCATTGATACTTGTTCAGGATT | | |
| | TCTTTTTGCTTCTCTGCATACAGGAGAAGCTTCAAAAAACGTAATTGATCATTGCCTACAAGCATTTAATGCCATGGGATTGCCTAAACTTATTAAGACAGACA | | |
| | ATGGGCCATCTTATTCCAGTAAAAACTTTATTTCATTCTGTAAAGAATTCGGTATTAAACATAAAACTGGAATTCCTTACAACCCCATGGGACAAGGAATAGTT | | |
| | GAACGTGCTCATCACACCTTAAAGAATTGGCTTTTTAAGACAAAAGAGGGGCAGCTATATCCCCCAAGGTCACCAAAGGCCCACCTTGCCTTCACCTTATTTGT | | |
| | CCTAAATTTCTTGCACACCGATATCAAGGGCCAGTCTGCAGTGGATCGCCACTGGCATCCAGTTACTTCTAATTCTTATGCATTGGTAAAATGGAAGGACCCCC | | |
| | TGACTAATGAATGGAAGGGTCCAGATCCAGTTCTAATTTGGGGTAGGGGCTCAGTTTGTGTTTTTTCACGAGATGAAGATGGAGCGCGGTGGCTGCCAGAG | | |
| | AGATTAATTCGTCAGATGAACACAGATTCTGACTCTTCTGGTAAGTATCATTCTAAAGACTAAAATTCCTTTTGTGCTTAAAATTCAGCTGAGAGCAACAGCTC | | |
| | TCAAAGCTGTTCTCCAGCTACTCTCTGAGCCAGCTCCCGAAAGGAGGCCGGAGACTAGCCTCAGCTTTACAATTTGCATTTGAATAAAGTACCTAGACTTCCCT | | |
| | GAAAGAAGTTCTGCTTTCCTACTTTCTCACTGTCTTTCAAGATTTTGTCTTTCAAGCAGGTAAATCAACACTCTCGAAGCGGACCAGCGGATGTGCATCCCCGC | | |
| | CCCCCTAGAGCACACAGGTGGCAGCTGTTATCCCCAGACTCAGGACATTTCCAGCATGTGGCTTTCAGTCTGAGTTAAAAATTTAGGTTTACCTAGAGGGCTA | | |
| | GAAGAGTAGATTTTTCTATATTAATAAAGATTGGTTTTTATTTTGATAGACAGGCTTAGCCCCTTAGCTGACCTCTGGCTTTTCACCCTTGCTGTTACTGCAAGG | | |
| | TGTCCTTAGCTCAATAGGCTGTGGAAAAAACAGGGATGAGGAGAAACGACTTCCAGCTCCTATTTTACCCATAAATCGTGGTGTTATTAACGACATAATTCTT | | |
| | GCTTAGGCTTTGCTAATTCTGAGGTTGATAATTCTCCTTTAGGAGCTGCACAGCACTCAGAACTGTGCATACTGGTTTGTGATTGTACAAATTCAGTATGGGCA | | |
| | CCGCTTGGTGCAGAAGTACTGCAAGGGAAGGTCCGGCTTGACCGTTTCTGAGTTTCCTGTGAGATAAACCCGGTTTAAAAGAGGTTGGTATCATATTTTGGTT | | |
| | AAAAATAAAAAATATTTTCCGGCTCTACCTCGCCTCCCCAAAAGGTACCGAGAGCCACATGTGTGGGTTTTACCCACGGGAGGAATCGGGTCCATGTCCACCC | | |
| | AAGCCAAGGTTAAAAGCCCACTCATCTACGGATGAGAAAATCATTTGATCACCTCAGTTAAGCGTTGCCTTATTTAACTTAATTAATAGGGGGGAGAGAGATT | | |
| | GGAGACGTACTATTGAAAGGTCAAGCCCTTCACTGCCTCCCACCCAAATAAAAAAGCCAATTGGCCTTGTACTACAAGAGCCGGTCACTCCTTCTCCCTGTTTC | | |
| | CCACCTATCATCCAAAAATGCGGAGATCAACTAGAAAGTTACCTACCAAGTACTAATTAGCATCTTAAAGTCAGAGCCTGCAGCCCCGGGCCTTTCAGTTAGT | | |
| | TGTTTACTAGAAAGGACAGTCTTAAGCCAGATACAGTTTACCATAAGAAAGGTTAAAGAATCCTAGTGAAGCAAGTTTTTTCTTTAGCCCTAGATTCCAGGCA | | |
| | GAACTATTTGAGCATAGATAATTTTTCCCCCTCAGGCCAGCCTTTTCTTTTTTTTTTATTTTGTTAATAATAGGGAGGAGATGTAGTCTCCCCTCCCCCAGCCTGA | | |
| | AACCTGCTTGCTCGGGGTGGAGCTTCCTGCTCATTCGTTCTGCCACGCCCACTGCTGGAACCTGAGGAGCCACACACGTGCACCTTTCTACTGGACCCGAGAT | | |
| | TATTCGGCGGGAATCGGGTCCCCTCCCCCTTCCTTCATAACTAGTGTCGCAACAATAAAATTTGAGCTTTGATCAGTATGAATTTGTCTTAGCTCCGTTTCTTCT | | |
| | TTTGCCCCGTCTAGATTCCTCTCTTACAGCTCGAGCGGCCTTCTCAGTCGAACCGTTCACGTTGCGAGCTGCTGGCGGCCGCAACA | | |
| ETnII | TGTAGTCTCCCCTCCCCCAGCCTGAAACCTGCTTGCTCAGGGGTGGAGCTTCCTGCTCATTCGTTCTGCCACGCCCACTGCTGGAACCTGCGGAGCCACACCCG | 375 | 7076 |
| A1 | TGCACCTTTCTACTGGACCAGAGATTATTCGGCGGGAATCGGGTCCCCTCCCCCTTCCTTCATAACTAGTGTCGCAACAATAAAATTTGAGCCTTGATCAGAGT | | |
| | AACTGTCTTGGCTACATTTTTTCTTCCGCCCCGTCTAGATTCCTCTCTTACAGCTCGAGCGGCCTTCTCAGTCGAACCGTTCACGTTGCGAGCTGCTGGCGGCCG | | |
| | CAACATTTTGGCGCCAGAACTGGGACTTGAAGAATGGCAGAGAGATGCTAAGAGGAACGCTGCATTGGAGCTCCACAGGAAAGGATCTTCGTATCAGACAT | | |
| | CGGAGCAACGGACAGGTACACATGCTAGCGCTAGCTTAAAATTTCAGTTTTGTAAAGTGTTGCTGAGGATGCGGTAGGATACGAATTAAGCTTGAATCAGTG | | |
| | CTAACCCAACGCTGGTTCTGCTTGGGTCAGCAGCGTGTTAATCGGAACTAGAAACGGAAACAGGCAGGTTAGCCGCAGCTTTTTAGGAAGCTGCTTAGGTGG | | |
| | AAGAAGAAAGGGTTTAAAGTCATGGATCAGGCGGTAGGCCGTAGCTCTCCGAAGCTACATGAGGTGTGAGAAAAGAAAGGGTTTATTAAAAGGAATAGGC | | |
| | GTATTGCCCCAGTTAATAAAAAATGCATCATAAGGGAGGAAAATGTCCCAAAAAGCAGAGAGAAATATCTCTCTGGGCCTTATAGCAGGAGTACTCTGTTAC | | |
| | CTTTTGTGTCTTGTCTAATGTCCGGTGCACCAATCTGTTCTCGTGTTCGATTCATGTATGTTCGTGTCCAGTCTGTATGAATGAATGTTCTATGTTTTGTGTTGGA | | |
| | TAATAAAGATGGTATAAAAAACTTTATCTGCAAAGCCGAGAGCTGCCACGTGTTTCAGCCAGGAATCAGACACGTGGCGAGAGGGCCCCTGCTGGAAAAAC | | |
| | TGTTCGTTTTAGGAAATAAGGGCGAGTGCACAGCCTCTAAGTTTCAGAGTAAAAAAGCTAATAAATGGTTCATGATTAATGTGTTTGACAATGGTAAAGTGTT | | |
| | TTTTATTTTATGATTGTAGCTACAAAAATTATTATTCTCTGATTGGTCTAAATGTAACTGCTTCATTTGGTTCCTTTTTATTGGTAATGTGCTCTAAGTGTTTTCAC | | |
| | AATCAGCTCATAAGTTGTTGGTTAAGATTAATAATTGTTACATTGCTACAGATGGTTAGTGTTAAATTTGATAACTCAAGTTTAGAGTCCTTCCGACACATGGC | | |
| | ATAAGGCAGCCCAAGAGGCTGTGTCTCTAAAGATATTTCTAGTTTAGGTAATAATTAATGTGGATCGTATCCTAAATAGTAAAATTTAAAATAAGATTTAAAG | | |
| | CAATGTCTCTTTATTAAAGCATTAAAGCTTGCTTTAATAGGTATTCATACGTATTAATTGACATCCAAACTTCGTAATATGATAGTAATGCTTCTAATATTGATTT | | |
| | TAAAGATAATAAATTGTTTTAAACTTGGGTTTTGCTTTCCCAAGGTTACAGGTATTATCCTAACCTTGCACAAAAACTTAAAAATTATGGTTAAAACTGCTATTG | | |
| | TTCCATTGACTGCAGCTTGCAGTTTGATTTCAAATTTAAGATCTTTAATTCACCTGTATACTGTAATTAAGATAATTACAAGAGTAATCATCTTATGAGAGCGCT | | |
| | CATACAGCTCACTTCATACAAAACTGTGACAGAGTTATCTAGTTATGTTTGTCTTTGTGAATAGATTAGATAAACAGTTTGGTTATAGTTGCTGCCTGGCAGGA | | |
| | AACTGTAGTACAGGCAATTTTTTCACAACACTAAAGTTGTGAAGAACGTTTTAACTGTAAGTCATCTTAAGAAAGAGTATCAAAATTTAGAGGCGTAGACAGT | | |
| | TATATTGTTTCTCTAAAATCGGTCCTTATTACAGGAGGGCCAGGATGCTCAGATTAAAAAGTATTTTACGTTTGAGTCAATGCAGGGCTCTGGGCAGCCCCAG | | |
| | AGCGGCTTGTGGCCTTTCTTTTGTTTTGCAAACAGTGCCTGAAAAAAATTTTTCCCTGTGTTCAAGAGAAATTCTTTTTAACAGTGTTGCAGATCTATTCAGATG | | |
| | TTTAAAATAAGACTTAAATTCAAAGAGTTTTGCTTCTAGTGAACTGTAATCACTAGAAAATTTTGCCTCTAAGTATGGCTAATGTAACTTTACATTATGTAAGAA | | |
| | AAAATTTTATTGTTTCTGCTTCTATACAAGAAGCCAAGAGTTTTAATCTTTCAGTGTATATTGTTTCCTAAGTGAAAAGTATTTTATTAACTAATGCTTCTTGAAG | | |
| | TTTACCTTAAATCCTTGCTCTCACCCAAAAGATTCAGAGACAATATCCTTTTATTACTTAGGGTTTTAGTTTACTACAAAAGTTTCTACAAAAAATAAAGCTTTTA | | |
| | TAATTGTTATTAATTGGTAATTAAAAATTGGTTGTGCCCAAAACAATTCTTTGGCCAAAAAGAAAACATTATTGTAAAGTCATTTTTCTCATCCTCCCAGCCAAT | | |
| | CGTTGGCCCACGTGGGCCCAACTAGCTTCTGTGGGGTGGAGTCTTAAGACACAGTTTCCCCTGTTCCAGCACAGATGATCTAGTTGTGTGCTGTAGATGGTGT | | |
| | TTTAAAATACTGAACAATCAAACCTTAATTTGTATATTAATAGTCAATGCCATATCTCTGAGCTCGCAATTGCTTAAATTGTTCATCCCTCAGATACTATTAATTC | | |
| | TCAAATTTACAATTGCTTATGCATATTTCTAGTTAATAAATAAATAAATTATGCACATGTAACTCTTAATAACTTTACAAGCCTTCTAGTTACAACTGCTCCTTAA | | |
| | GAAAATTGATTGAAAGTGCAATTAGTCACTGCCCCTTTACAGCCATGTATTTAAAATGTTTTGTCAACTAGTTATTAATTCAAAAGTTTAGGTATTGTAAAATTT | | |
| | TAAAACTTTAACTTCTTAAAAGACAAAAAAGAGAGAAATTGTATCCCCGTGCATACTATTTAGTGCATTCCCATGCACACTATCTAAGTCTTACCTTTATTTTCA | | |
| | AAATTTAATATTTTAATGTCAAAGAGTTTAATAAAGGCTTTATAGTATCTTAAAGGGATCTACTTATTGGCTTATAGATTAATCCTAAAACAACTACCTTATTAG | | |
| | AAAAGGGAAAAACAGGTTTTCTCCACAGAACGCTGCAGAAGCATATTAGTAAATTCGTGTGACGAGCTGGTAGGTAAGTTGACTCATGTCCTGATTGAATTG | | |
| | ACTAAAAAACTAAATTAAATTCATGTTTTAGATCCATCCTTACTTGTCATTTTTCCAGTTTAGACTAGCTTCTAGCCTTTTAACTTTATGACAATAGTACATCAGA | | |
| | GACTGTATATTCAGACTTAGTAAAATTAGTCATTTAATAGAGTCATAATGATTTTTCTCTTTTCTTCAGTGTGACCAGCTCTTCTAACTCAATCTTAGACTGGTCT | | |
| | AATATTCAGTCCAATGTTAGAGATTCCTATATTCTAAATTACCTAGCAAGTTAATTCAGAACACATCCTCCACTTCCCCTAGATCCCTAACCTCAGAAGGATTAC | | |
| | TGGCCTTACAGCTAGTGGAAAAGGCTATTGAAGAACAGTTTGTCACTTACATAGATTACTCCCTGCCGCTGCACCTGTTAATTTTTAATACGACTCATGTGCCT | | |
| | ACGGGATTGCTATGGCAAAAATTTCCTATAATGTGGATACATTCGAGGATTTCTCCCAAACGTAATATCTTGCCATATCACGAAGCAGTGGCTCAGATGATTAT | | |
| | CACTGGAATAAGGCAGGCATTGACTTATTTTGGAAAGGAGCCAGATATCATTGTCCAGCCTTACAGCGTGAGTCAGGACACTTGGCTGAAACAGCATAGTAC | | |
| | AGATTGGTTGCTTGCACAATTAGGGTTTGAAGGAACTCTAGATAGCCACTACCCCCAAGATAGGTTGATAAAATTCTTAAATGTGCATGATATGATATTTCCTA | | |
| | AGATGACTTCCTTACAGCCTTTAAATAATGCTCTATTGATTTTTACTGATGGCTCCTCTAAAGGGCGAGCTGGATATCTTATTAGTAATCAACAGGTTATCGTA | | |
| | GAGACTCCTGGTCTCTCGGCTCAGCTCGCCGAATTAACAGCAGTACTGAAGGTTTTTCAGTCTGTGCATGAGGCTTTTAATATTTTTACTGACAGTTTATATGTT | | |
| | GCTCAGTCAGTACCCTTACTGGAAACCTGTGGTACGTTTAACTTCAATACGCCGTCAGGATCTTTATTTTCAGAATTACAAAACATTCTCGCCCGGAAAAATCC | | |
| | GTTTTATATTGGCCACATACGGTCTCACTCTGGTCTTCCTGGACCTCTGGCAGAGGGTAATGATCGCATTGACAGAGCTCTAATAGGAGAAGCCTTAGTTTCA | | |
| | GATCGGGTTGCTTTGGCCCAACGTGATCACGAAAGGTTTCATCTTTCTAGCCATACCCTAAGACTCCGACATAAGATCACAAAAGAGCAAGCGAGAATGATC | | |
| | GTAAAACAATGTCCTAAATGTATTACTTTATCTCCAGTGCCGCATCTAGGAGTTAATCCTAGAGGCCTTATGCCTAATCATATTTGGCAAATGGATATAACCCA | | |
| | TTATACAGAATTTGGAAAACTAAAATATATACATGTCTACATTGATACTTGTTCAGGATTTCTTTTTGCTTCTCTGCATACAGGAGAAGCTTCAAAAAACGTAAT | | |
| | TGATCATTGCCTACAAGCATTTAATGCCATGGGATTGCCTAAACTTATTAAGACAGACAATGGGCCATCTTATTCCAGTAAAAACTTTATTTCATTCTGTAAAG | | |
| | AATTCGGTATTAAACATAAAACTGGAATTCCTTACAACCCCATGGGACAAGGAATAGTTGAACGTGCTCATCGCACCTTAAAGAATTGGCTTTTTAAGACAAA | | |
| | AGAGGGTCAGCTATATCCCCCAAGGTCACCAAAGGCCCACCTTGCCTTCACTTTATTTGTCCTAAATTTCTTGCACACCGATATCAAGGGCCAGTCAGCAGCG | | |
| | GATCGCCACTGGCATCCAGTTACTTCTAATTCTTATGCATTGGTAAAATGGAAAGACCCCCTGACTAATGAATGGAAGGGTCCAGATCCAGTTCTAATTTGGG | | |
| | GTAGGGGCTCAGTTTGTGTTTTTTCATGAGATGAAGATGGAGCGCGGTGGCTGCCAGAGAGATTAATTCGTCAGATGAACACAGATTCTGACTCTTCTGGTA | | |
| | AGTATCATTCTAGAGACTAAAATTCCTTTTGTGCTTAAAATTCAGCTGAGAGCAACAGCTCTCAAAGGTGTTCTCCAGCTACTCTCTGAACCAGCTCCCGACAG | | |
| | GAGGCCGGAGACTAGCCTCAGCTTTACAATTTGCATTTGAATAAAGTACCTAGACTTCCCGGAAAGAAGTTCTGCTTTCCTACTTTCTCACTGTCTTTCAAGATT | | |
| | TTGTCTTTCAAGCAGGTAAATCAACATTCCCGAGGCGGACCAGTGGATGTGCATCCCTGCCCCCCTAGAGCACACAGGTGGCAGCTGTTACCCCCAGTCTCAG | | |
| | GACATTTCCAGCATGTGGCTTTCAGTCTGAGTTAAAAATTTAGGTTTACCTAGAGGGCTAGAAGAGTAGATTTTTCTATATTAATAAAGATTGGTTTTTATTTT | | |
| | GATAGACAGGCTTAGCCCCTTAGCTGACCTCTGGCTTTTCACCCTTGCTGTTACTGCAAGGTGTCCTTAGCTCAATAGGCTGTGGAAAAAACAGGGATGAGGA | | |
| | GGAACGACTTCCAGCTCCTATTTTACCCACAAATCGTGGTGTTATTAACGACATAATTCTTGCTTAGGCTTTGCTAATTCTGAGGTTGATAATTCTCCTTTAGGA | | |
| | GCTGCACAGCACTCAGAACTGTGCATACTGGTTTGTGATTGTACAAATTCAGTATGGGCACCGCTTGGTGCAGAGATACTTACTGCAAGGGAAGGTCCGGCT | | |
| | TGACCATTTCTGAGTTTCCTGTGAGATAAACCCGGTTTGAAAGAGGTTGGTACCAAATTTTGGTTAAAAATAAAAAATATTCTCCGGCTCTACCTCGCCTCCCC | | |
| | AAAAGGTACCAAGAGCCACATGTGTGGGTTTTACCCACGGGAGGAATCGGGTCCATGTCCACCCAAGCCAAGGTTAAAAGCCCACTCATCTACGGATGAGA | | |
| | AAATCATTTGATCACCTCAGTTAAGCGTTGCCTTATTTAACTTAATTAATAGGGGGGAGAGAGATTGGAGACGTACTATTGAAAGGGCAAGCCCTTCACTGCC | | |
| | TCCCACCCAAATAAAAAGGCCAATTGGCCTTGTACTACAAGAGCCGGTCACTCCTTCTCCCTGTTTCCCACCTATCTTCCAAAAATGCGGAGGAATATCAACTT | | |
| | AGTGCTATTTTCACATCGTTCAGTCAAACTTAGCCAGAGTTCCAACGCCCTACTTAAAATTCAACTAGAAAGTTACCTACCAAGTACTAATTAGCATTATAAAGT | | |
| | CAGAGTCTGCAGCTCCAGGCCTTTCAGTTAGTTGTTTACTAGAAAGGACAGTCTTAAGCCAGATACAGTTTACCATAAGAAAAGTTAAAGAATCCCAGTGAAG | | |
| | CAAGTTTTTTCTTTAACCCTAGATTCCAGGCAGAACTATTGAGCATAGATAATTTTTCCCCCCTCAGGCCAGCTTTTTCTTTTTTTATTTTGTTAATAATAGGGAG | | |
| | GAGATGTAGTCTCCCCTCCCCCAGCCTGAAACCTGCTTGCTCAGGGGTGGAGCTTCCTGCTCATTCGTTCTGCCACGCCCACTGCTGGAACCTGCGGAGCCAC | | |
| | ACCCGTGCACCTTTCTACTGGACCAGAGATTATTCGGCGGGAATCGGGTCCCCTCCCCCTTCCTTCATAACTAGTGTCGCAACAATAAAATTTGAGCCTTGATC | | |
| | AGAGTAACTGTCTTGGCTACATTTTTTCTTCCGCCCCGTCTAGATTCCTCTCTTACAGCTCGAGCGGCCTTCTCAGTCGAACCGTTCACGTTGCGAGCTGCTGGC | | |
| | GGCCGCAACA | | |
| ETnII | TTCTACAAAAGGCTATACTCAACAAAACTGGAGAACCTGGAAGAAATGGAGATGTTTCTAGACAGATACCAGGTACCAAAGTTGAATCAAGATCAGGTTAAT | 376 | 5543 |
| B1 | GATCTAAACAGTCCTATATCCCCCAAAGAAATAGAAGCAGTCATTAATAGTCTCCCAACCAAAAAAATCCCAGGACCAGATGGGTTTAGTGCAGAGTTCTATC | | |
| | AGACCTTCAAAGAAGATCTAATCCCAGTTCTTCACAAACTATTCCACAAAATAGAAACAGATGGTACTCTACCCAACAAATTCTATGAAGCCACAATTACTCTG | | |
| | ATACCTAAAACACAAAAAGACCCAACAAAGATAGAGAACTTCAGACCAATTTCCCTTATGCATATTGATGCAAAATCCTCAATAAGGTTCTTGCTCACTGAATC | | |
| | CAAGAACACATCAAAACAATCATCCCTCCTTACCAAGTAGGTTTCATCCCAGCGATGCAGGGATGGTTCAATATATGGAAATCCATCAACATAATCCAGTATAT | | |
| | AAACAAACTCAAAGACAAAAACCACATGATCATCTCGTTAGATGCTGAGAAAGCATTTGACAAAATCCAACACCCATTCATGATAAAAGTCTTGGAAAGATCA | | |
| | GGAATTCAAGGCCCATACCTAAACATGATAAAAGCAATCTACAGCAAACCAGTAGCCAACATCAAAGTAAATGGTGAGAAACTTGAAGCAATACCACTAAAA | | |
| | TCAGGGACTAGACAAGGCTGTCCACTCTCTCCCTACCTACTCAACATTGTACTTGAAGTCCTAGCCAGAGCAATTAAACAACAAAAGGAGATCAAGAGGATAC | | |
| | AAATTGGAAAGGAAGAAGTCAAAAATATCACTCTTTGCAGATGATATGATAGTATATATAAGTGACCCTAAAAATTCCACCAGAGAACTCCTAAGCCTGATAA | | |
| | ACAGCTTCGGTGAAGTAGCTGGATATAAAATTAACTCAAACAAGTCAATGGCCTTTCTGTACACAAAGGATAAATAGGCTGAGAAAGAAATTAGGGAAACAA | | |
| | CACCCTTCTCAATAGTCACAAATAATATAAAATACCTTGGCGTGAGTCTAACTAAGAAAGTGAAAGATCTGTATGATAAGAACTTCATGTCTCTGAAGAAAGA | | |
| | AATTAGAGAAGATCTCAGAAGATGGAAAGATCTCCCATGCTCATGGATTGGCAGGATCAATATAGTAAAAATGGCTATCTTGCTAAAACCATCTAAAGATTCA | | |
| | ACGCAATCTCCATGAAAATTCCAACTCAATTCTTCAACAAATTAGAAAGGGCAATCGGCAGATTCATCTGGAATAACAAAAAACCTAGGATAGCAAAAACTCT | | |
| | TCTCAAGGATAAAAGAACCTCTGGTGGAATCACCATGCCCGACCTAAAGCTGTACTACAGAGCAATTGTGATTAAAAACTGCATGGTACTGGTATAGTGACA | | |
| | GACAAGTAGACAAATGGAACAGAATTGAAGACCCAGAGATGAACCCACACGTCTATGGTCACTTGATCTTTGACAAGGGAGCTAAAACCATCCAGTGGAAA | | |
| | AAAGACAGCATTTTCAACAAATGGTGTTGGCACAACTGGCTGTTATCATGTAGAAGATTGTGAATTGATCCATTCATATCTCCTTGTACTAAGATCAAATCTAA | | |
| | GTGGATCAACATAAAACCAGAGACAGTGAAACTTATAGAGGAGAAAGTAGGGAAAAGCCTCGAAGATTTGGGTACAAGAGAAAAATTCCTGAATAGAACA | | |
| | GCAATGGCTTGTGCTATAAGATCGAGAAACGATAAATGAGACCTCATAAAATTGCAAAGCTTCTGCAAGGCAAAAGACACCGTCAATAAGACAAAAGACCAC | | |
| | CAACAAATTGGGAAAGGATCTTTACCTATCCCAAATTGGATAGGGTACTAACATCCAATATATATAAAGAACTCAAGAAGGTGGACTCCAGAAAATCAAATA | | |
| | ACCCCATTAAAAAATGGGGCTCAGAACTGAACAAAGAATTCTCACCTGTGGAATACCGAATGGCAGAGAAGCACCTGAAAAAATGTTCAACATACTTAACCA | | |
| | TCAGGAAAATGCAAATCAAAACAACCCTGAGATTCTACCTCACACCAGTCAGAATGGCTAAGATCAAAAATTCAGGTGAGAGCAGATGCTGGTGAGGATGT | | |
| | GGAGAAAGAGGAACACTCCTCCATTTTTGGTGGGATTGCAAGCTTGTACAACCACTCTGGAAATCAGTCTGGTGGTTCCTCAGAAAATTGGACATAGTACTAC | | |
| | CGGAGGATCCAGCAATACCTCTCCTGGGCATATATCCAGAAGATGTCCCAACCGGTAAGAAGGACACTATGTTCATAGCAGTGTTATTTTTAATAGCCAGAAG | | |
| | CTGGGAAGAACCCAGATGCTCCTCAACAGGGGTGGATACAGAAAATGTGGTACATTTACACAATGGAGTACTACTCAGCTATTAAAAAGAATGAATTTATGA | | |
| | AATTCCTAGGCACATGGATGGACCTGGAGGGCATCATCCTGAGTGAGGTAACCCAATCACAAAGGAACTCACACAATATATACTCACTGATAAGTGGATATT | | |
| | AGCCCAGAATCTTAGGATACCCAAGATATAAGATACAATTTGCTAAACGCATGAAACTCAAGAAGAATGAAGACGAGGGTGTGGGGAGTTTGCCCCTTCTTA | | |
| | GAATTGGGAACAAAAGGGGCGTGGGGGGAGTTACAGAGACAAAATTTTGAGCTGTGATGAAAGGCAATCTAGTGATTGCCATATCCAGGGATCCATCCCAT | | |
| | AATCAGCTTCCAAATGTTGACACCATTGCATACTCTAGCCAGATTTTGCTGAAAGGACTCAGATATAGCTGTTTCTTGTGAGACAATGCCGGGGCCTAGCAAA | | |
| | CACAGAAGTGGATGCTCCCAGTCAGCTATTGGATGGATCACAGGGCCCCCAATGCAGGAGCTAGAGAAAGTACCCAAGGATCTAAAGGGATTTGCAACCCT | | |
| | ATAGGTGGAACAACATTATGAACTAACCAATACCCCGGAGCTCTTGTCTCTAGCTTCATATGTATCAAAGGATGGCCTAGTTGGCCATCATTGGAAAGAGAGG | | |
| | CCCATTGGACTTGCAAACTTTATATGCCTCAGTACAGGTGTAAGCCAGGGCGAAAAAGGGGGAGTGGGTGGGTAGGGGAGTAGGGGGGTGGGTATGGGG | | |
| | GACTTTTGGGATAGCATCGGAAATGTAAATGAGGAAAATACCTAATTAAAAAAAAAGAGTTTGAACTAGAAGGAATAAGTTTTGCCTAGCATCAGGGAGGT | | |
| | CTATATGTGGTTTGGGTGCCCAGAAAGACTGAGTAGTTATAATGCCAGAGGTTTCAAAAATAGGCCAAAGAGAGGCAGATTAGTAATCTCTGTACTGTAATT | | |
| | GCTGTGGTGGTAACACCTGCATGACCAAATAAATCTCTGGGGTTTTTGGTCCTTCGAATGGTGAAAATCATACATATTTCCACATCTTGGAGTCAAAACTTTTG | | |
| | TTGTGTGATACTGATATTCATTCCTGAGCCATTTTCCTAAGCTTATTGGTTGTGTGTGATATAATATTGTCTAGATGTCTAGACAGTGCTTGCCTTGGTACAGTA | | |
| | CTTGGTGAGAACTCACTGTCCCACCTGCTTGGCTAACCCCCAGTTTTAGTGATTTTTTTTTTTTTACTAAGCTTCAACATTTACAATAAACACATCTTAAAAAGTG | | |
| | TCGAAAGAAGTAGTATATTAGAAATTGGAGTGCATTGGATAGATGAAAGGTTAAGGTCTTTGAAATGAGGTAGGAAGATTTTAGAACACAAAGGACCATGT | | |
| | TAACCAGAGGGACATTGTGTGCTCTTGCTCCTTTCCTTCTGATCATAAGTCAAGATGGTTTGGAGCAGATATTTTGGGATTAATACCCATTCATTCACTCATTTC | | |
| | TTGAGTTCTTTCTATAATTTGTCTTCTGACACATTGTCCTCAGCAATTCCAAAGTTTAGGTTAACCCTAAAAACTTTTTCCTTCAAAGTCAATTTTGTGAGGAGTG | | |
| | GTAGAACATGATATTGGCAATTTATGCACAGACTTTTACGTGTTTAGGTACTGTCTTGAACTCTTTTCATGTTTCTTTGGTCTGCCTTCTCCAGTTTTGTGGATGT | | |
| | TTATAATTGTAGTCTCCCCTCCCCCAGCCTGAAACCTGCTTGCTCGGGGTGGAGCTTCCTGCTCATTCGTTCTGCCACGCCCACTGCTGGAACCTGAGGAGCCA | | |
| | CACACGTGCACCTTTCTACTGGACCAGAGATTATTCGGCGGGAATCGGGTCCCCTCCCCCTTCCTTCATAACTGGTGTCACAACAATAAAATTTGAGCCTTGAT | | |
| | CAGAGTAACTGTCTTGGCTACATTTCTTCTTTTGCCCCGTCTAGATTCCTCTCTTACAGCTCGAGCGGCCTTCTCAGTCGAACCGTTCACGTTGCGAGCTGCTGG | | |
| | CGGCCGCAACATTTTGGCGCCCGAACAGGGACCTGAAGAATGGCAGAGAGATGCTAAGAGGAACGCTGCATTGGAGCTCCACAGGAAAGGATCTTCGTATC | | |
| | GGACATCGGAGCAACGGACAAGTACACATGCTAGCGCTAGCTTAAAATTTCAGTTTTGTAAAGTGTTGCTGAGGATGCGGTAGGATACGAATTAAGCTTGAA | | |
| | TCAGTGCTAACCCAACGCTGGTTCTGCTTGGGTCAGCAGCGTGTTAATCGGAACTAGAAACGGAAACAGGCAGGTTAGCCGCAGCTTTTTAGGAAGCTGCTT | | |
| | AGGTGGAAGAAGAAAGGGTTTAAAGTCATGGATCAGGCGGTAGGCCGTAGCTCTCCGAAGCTACATGAGGTGTGAGAAAAGAAAGGGTTTATTAAAAGGA | | |
| | ATAGGCGTATTGCCCCAGTTAATAAAAAATGCATCATAAGGGAGGAAAATGTCCCAAAAAGCAGAGAGAAATATCTCTCTGGGCCTTATAGCAGGAGTACTC | | |
| | TGTTACCTTTTGTGTCTTGTCTAATGTCCGGTGCACCAATCTGTTCTCGTGTTCGATTCATGTATGTTCGTGTCCAGTCTGTATGAATGAATGTTCTATGTTTTGT | | |
| | GTTGGATAATAAAGATGGTATAAAAAACTTTATCTGCAAAGCCGAGAGCTGCCACGTGTTTCAGCCAGGAATCAGACACGTGGCGAGAGGGCCCCTGCTGG | | |
| | AAAAACTGTTCGTTTTAGGAAATAAGGGCGAGTGCACAGCCTCTAAGTTTCAGAGTAAAAAAGCTAATAAATGGTTCATGATTAATGTGTTTGACAATGGTAA | | |
| | AGTGTTTTTTATTTTATGATTGTAGCTACAAAAATTATTATTCTCTGATTGGTCTAAATGTAACTGCTTCATTTGGTTCTTTTTTATTGGTAATGTGCTCTAAGTGT | | |
| | TTTCACAATCAGCTCATAAGTTGTTGGTTAAGATTAATAATTGTTACATTGCTACAGATGGTTAGTGTTAAATTTGATAACTCAAGTTTAGAGTCCTTCCGACAC | | |
| | ATGGCATAAGGCAGCCCAAGAGGCTGGGTCTCTAAAGATATTTCTAGTTTAGGTAATAATTAATATGGTTCGTATCCTAAATAGTAAAATTTA | | |
| ETnII | TCTTAGGTTTCCCCAAAGACAGAACCATCACCCCAAATCAGCAGTGAATTGTTTATAAACACAATGACCTTATTTCTGCCCCACCATCACTCCTTTCTAGTTTCTT | 377 | 5536 |
| B2 | ATATTTAATTAAACCAAAATCGATGAATATTGGTTTACAGACAATTCCACTAGGCTTCTCTCAGGAAACTAGCTAGCAACAGGAACCTAGCCATAGCTTACATC | | |
| | ATTACCTGCCACGAGGTGCTAGCCAGGTATGTAAGAGAATTGCTAGTACCCAGCTCTCACTCCATCTCCTATTCACTCTGCCCCCTACTTCAGTCTCTTTCTCTTC | | |
| | AGACACCACAAAGTCACCCCATGCAACAGATCTCATGGGAGAGGTTTATTGGGGGAGGAATATGTAGAGGCTACCTCTGGGTGAGGTTATTAGGGGAACGA | | |
| | GGATAGGGAGAGGAGACACTGCCTTTTTATGCAGGATATAATGCATACAGGTAGAAAGGATGCATGACTCGTGGCAACAGTAAAATTGTGTCACATCTTGAT | | |
| | GGCTGGTGATGACCTGTCCTGCTCTTGCAAGATTGCTGGAATTGGGGTTAGCAATGTTCTTAGCATTTCTTTTTCTTTTCTTTTCTTTTATTTTATTTTCTTTTCTT | | |
| | TTCTTTTTTCTTTTTTTGTTTCTTTGGGGTTTTTTTTGTTTTTTTTTTTGTTTGTTTGTTTGTTTTGTTTTTTGGGGGTTTTTTGTTTGTTTGTTTTTCAAGACAGTGTT | | |
| | TCTCTGTAGCCCTGGCTGGAACTCACTTTGTAGACCAGGCTAGCCTCGAAAGAATCCACCTGCCTCTGCTTCCCAAGTGCTGGAATTAAAGGCATACGCCACC | | |
| | ATGCCCGGCAGCATTTCTTTTAGGCTCCACTCAACAATTACCTGGCAATAGCCAGGTGGGCTTGGCTTGCTGTAAAAGAGGCTGTTTGGGCTCTCCTTGTTCTC | | |
| | TCTGCCCCCTTTTCTATCTTTGACTCTCTCTTTCTCTCTCTCTCTCTCTCTCTCTCTCTCTCTCTCTCTCTCTCTCTCTCTCTCTCACACACACACACTTTTTCCATCTCT | | |
| | TCCCTTCCCTTTCCACATGTCCCTGGCTTGCCTCTCTTCTCTTGCCCTCTTCTCCCTTCTCCTGTCCTCTCCTCACCTGTCTTCTTCTCTCTCTCTCTCTCTCTCTCTGT | | |
| | CTCTCTCTCAGTTTCAGTTTCTCTCTGCTTCTCTCTCTCTCTCTCTCTCTCTCTCTCTCTCTCTCTCTCTCACTCTCTCTCTCTGTCTTTCTCTGTCTCTACTCCTTTCTC | | |
| | CTTTCTTATCCCCCTTCCCATGCCCCCAAATAAACTGTACCTCAGAGGAAAGGGATACCTCAGCATGGGCCCACTGAGGCAACCCTTCCACCACACCATACCAC | | |
| | CCTGTATAAAACATATACTTGGTTTTTATAAAACACAACAGGTGAAGTTTTCCAACAGTTTCTTTTTAAAAGTGTTTTTATTACATTTACTTCACGTATGCACACT | | |
| | TGTGTTTGTATGTGTGTGTCTGTCTATCTGTCTGTGTGTGTGTCTGTGTGGCATGCCTATGGAGAGACCAATAAACTGAAGTTAGTTTTTTCCTTCTATTACTTG | | |
| | TTTCCCAGGTCTAAACATGAAAACATATTGTCAATCTTGGCAGCAAGTGCCTTTACCTATTGAGCCATCTCTCCAGTTCTTGTAAGGATTTTCTTGGTCTCTAAA | | |
| | TCAACATTCTTACACCTACTCCATTCAATATAATGTGGCATTTGTTTAACTTTCCCCCAGACCATGAAGTGATAAAGCTTGACTGAGCTCAACAAGGAAGATCA | | |
| | AAGGGAAGTGACATTTAAGTATCAGACTTGAGAAGCAAGCTTGAAAGCCTCTGAGTTCTGCTTGGTTATATTCATCACGTAGTCGTGAAAACCTACATATTCC | | |
| | ATTGGGTTAAAGAGCAGAAAATATGATACAGAAGATATGAGTCTTAAGTCTTGAACATTTAGATAAATAGATCTTATGAGAAATTCCTTCCACATCTCAAGAA | | |
| | CAAATACAGATGCAGAAATAAGAACTTTGTGCCTGTGAATAGAGGGTAGGAATGAAGAAACTGGAAAACCCTTAAATAATATTTTAGTTATGGTATGGTGTG | | |
| | GTGGAAGGGGTGCCTCAGTGGGTCCATGCTGAGGTATCCCTTTCCTCTGAGGTACAGTTTATTTGGGGGCATGGGAAGGGGGATAAGAAAGGAGAAAGGA | | |
| | GTAAAGACAGAGAAAGACAAAGAGAGTGAGCGAGAGAGAGAGAGAGAGAGAGCAAGAGAGAGAGAGGGAGAGAGAGAGAGAGAGAGAGAGAGAGAG | | |
| | AGAGAGAGAGAGAGAGAAGAAATAACAGAGGAAAGATATTTGAAAGTTGGGTTTAGAGTGTCTTAAATGTTGGTAATTAGTGATCAATTGGTATATAACAA | | |
| | GGAACATCAAACTCAGTGATTTAACTCTGCTCACTTCTTTTGTAGGTCAGAAATCCACCCATGGCTTAGCTGGGTACTCTGTGTAGTGGTACCCTAGCACAATT | | |
| | CAGGTTTTAGCTGAGGCTGTTATTTTATCATTGTGATAGGTGCCTCTCCCAACCTCATTCAAGGTGTTGGTAGGATTTATCACCTTGTGGTAGCATAATTGGAA | | |
| | TCCTATTTTCTTGCCAACTCTCTGAGGAAGGGGTGGTTAACATTTGGAAGGATTTCATTTACATATTTTCTGAAAGCACTGGAATTTGTTATTTCTTGGCACTCT | | |
| | GCAGGAGAGGGTTTAACACAAACGTTTTTGTTTTTATTTCTGTTTCTGTTTTACTGGCTTTATCAAATGATGCAAATCCACCAGAATAATAAAAATTAACATTGA | | |
| | TAAAGTAAAAAATCAATTAATATATGCATACAATAACAATTGGTGTGTTAATACCAAAGTTTGGAAGAGAAAAACCACCTACCAAAATCATCACAGCCAAATT | | |
| | AATTAAAGCAAGCAATACATGACAGTAAGCTTTTTTCTTCCTGTACATGGGCTGTCTCCAGCTCAAATAGGGATTGAGAGGTCAAAATTAGATGTAAGGAAGA | | |
| | CAATGTTTTTTATAGTTAAGGGTTAAGTGGTTTTCTAATGGGGGAATTTGGCAGACAAAGTAGGTCAGTTTACAAGAACAGAGCATAAGCATAACGAGTTAG | | |
| | AAAGGTGACCCCCCCAAAACAAAGGCATGATTACAAGGTGGTCTTAACAACAGGAAGCCATAACAAGGGAGTCCAACTAACTCCAGAGATTACCAGATGGC | | |
| | TAAAGGCAAATGGAAGAATCTTACTAACAGAAACCACTCATCACCATAAGAACCCAGCACTCCGAACTCAGCCAGTCCTGGATACCCCAAAACGCCTGAAAA | | |
| | GCTAGACCCGGAGTTAAAATCATATCTCATGATGATGGTAGAGGACATCAAGAAGGACTTTAATAACTCACTTAAAGAAATACAGGAGAACACTGCTAAAGA | | |
| | GGTACAAGACCATAAAGAAAAGCAGGAAAACACAACCAAACAGATGATGGAACTGAACAAAACCATAGAAGACGTAAAAAGGGAAGTAGACACTGTAGTC | | |
| | TCCCCTCCCCCAGCCTGAAACCTGCTTGCTCAGGGGTGGAGCTTCCTGCTCATTCGTTCTGCCACGCCCACTGCTGGAACCTGAGGAGCCACACACGTGCACC | | |
| | TTTCTACTGGACCAGAGATTATTCGGCGGGAATCGGGTCCCCTCCCCCTTCCTTCATAACTGGTGTCGCAACAATAAAATTTGAGCCTTGATCAGAGTAACTGT | | |
| | CTTGGCTACATTTCTTCTTTTGCCCCGTCTAGATTCCTCTCTTACAGCTCGAGCGGCCTTCTCAGTCGAACCGTTCACGTTGCGAGCTGCTGGCGGCCGCAACAT | | |
| | TTTGGCGCCAGAACTGGGACCTGAAGAATGGCAGAGAGATGCTAAGAGGAACGCTGCATTGGAGCTCCACAGGAAAGGATCTTCGTATCGGACATCGGAG | | |
| | CAACGGACAAGTACACATGCTAGCGCTAGCTTAAAATTTCAGTTTTGTAAAGTGTTGCTGAGGATGCGGTAGGATACGAATTAAGCTTGAATCAGTGCTAACC | | |
| | CAACGCTGGTTCTGCTTGGGTCAGCAGCGTGTTAATCGGAACTAGAAACGGAAACAGGCAGGTTAGCCGCAGCTTTTTAGGAAGCTGCTTAGGTGAAAGAA | | |
| | GAAAGGGTTTAAAGTCATGGATCAGGCGGTAGGCCGTAGCTCTCCGAAGCTACATGAGGTGTGAGAAAAGAAAGGGTTTATTAAAAGGAATAGGCGGATT | | |
| | GCCCCAGTTAATAAAAAATACATCATAAGGGAGGAAAATGTCCCAAAAAGCAGAGAGAAATATCTCTCTGGGCCTTATAGCAGGAGTACTCTGTTCCCCTTTG | | |
| | TGTCTTGTCTAATGTCCGGTGCACCAATCTGTTCTCGTGTTCGATTCATGTATGTTCGTGTCCAGTCTGTATGAATGAATGTTCTATGTTTTGTGTTGGATAATA | | |
| | AAGATGGTATAAAAAACTTTATCTGCAAAGCCGAGAGCTGCCACATGTTTCAGCCAGGAATCAGACACGTGGCAAGAGGGCCCCTGCTGGAAAAACTGTTCG | | |
| | TTTTAGAAAATAAGGGCGAGTGCACAGCCTCTAAGTTTCAGAGTAAAAAAGCTAATAAATGGTTCATGATTAATGTGTTTGACAATGGTAAAGTGTTTTTTAT | | |
| | TTTATGATTGTAGCTACAAAAATTATTATTCTCTGATTGGTCTAAATGTAACTGCTTCATTTGGTTCCTTTTTATTGGTAATGTGCTCTAAGTGTTTTCACAATCA | | |
| | GCTCATAAGTTGTTGGTTAAGATTAATAATTGTTACATTGCTACAGATGGTTAGTGTTAAATTTGATAACTCAAGTTTAGAGTCCTTCCGACACATGGCATAAG | | |
| | GCAGCCCAAGAGGCTGGGTCTCTAAAGATATTTCTAGTTTAGGTAATAATTAATATGGTTCGTATCCTAAATAGTAAAATTTAAAATAAGATTTAAAGCAATGT | | |
| | CTCTTTATTAAAGCATTAAAGCTTGCTTTAATAGGTATTCATAGGTATTAATTGACATCCAAACTTCGTAATACGATAGTAATGCTTCTAATATTGATTTTAAAG | | |
| | ATAATAAATTGTTTTAAACTTGGGTTTTACTTTCCCAAGGTTATAGGTATTATCCTAACCTTGCACAAAAAACTTAAAAATTATGGTTAAAACTGCTATTGTTTC | | |
| | ATTGACTGCAGCTTGCAGTTTGATTTCAAATTTAAGATCTTTAATTCACCTGTATACTGTAATTAAGATAATTACAAGAGTAATCATCTTATGAGAGTGCTCATA | | |
| | CAGCTCACTTCATACAAAACTGTGACAGAGTTATCTAGTTATGTTTGTCTTTGTAAATAGATTAGACAGTTTGGTTATAGTTGCTGCCTGGCAGGAAACTGCAG | | |
| | TACAGGCAATTTTTTCACAACACTAAAGTTGTGAAGAACGTTTTAACTGTAAGTCATCTTAAGAAAGAGTATCAAAATTTGGAGGCGTAGACAGTTATATTGTT | | |
| | TCTCTAGAATCGGTCCTTATTACAGGAGGGCCAAG | | |
| ETnII | TGTAGTCTCCCCTCCCCTAGCCTGAAACCTGCTTGCTCGGGGTGGAGCTTCCTGCTCATTCGTTCTGCCACGCCCACTGCTGGAACCTGAGGAGCCACACACGT | 378 | 5538 |
| B3 | GCACCTTTCTACTGGACCAGAGATTATTCGGCGGGAATCGGGTCCCCTCCCCCTTCCTTCATAACTGGTGTCGCAACAATAAAATTTGAGCCTTGATCAGAGTA | | |
| | ACTGTCTTGGCTACATTTCTTCTTTTGCCCCGTCTAGATTCCTCTCTTACAGCTCGAGCGGCCTTCTCAGTCGAACCGTTCACGTTGCGAGCTGCTGGCGGCCGC | | |
| | AACATTTTGGCGCCCGAACAGGGACCTGAAGAATGGCAGAGAGATGCTAAGAGGAACGCTGCATTGGAGCTCCACAGGAAAGGATCTTCGTATCGGACATC | | |
| | GGAGCAACGGACAAGTACACATGCTAGCGCTAGCTTAAAATTTCAGTTTTGTAAAGTGTTGCTGAGGATGCGGTAGGATACGAATTAAGCTTGAATCAGTGC | | |
| | TAACCCAACGCTGGTTCTGCTTGGGTCAGCAGCGTGTTAATCGGAACTAGAAACGGAAACAGGCAGGTTAGCCGCAGCTTTTTAGGAAGCTGCTTAGGTGAA | | |
| | AGAAGAAAGGGTTTAAAGTCATGGATCAGGCGGTAGGCCGTAGCTCTCCGAAGCTACATGAGGTGTGAGAAAAGAAAGGGTTTATTAAAAGGAATAGGCG | | |
| | GATTGCCCCAGTTAATAAAAAATACATCATAAGGGAGGAAAATGTCCCAAAAAGCAGAGAGAAATATCTCTCTGGGCCTTATAGCAGGAGTACTCTGTTCCC | | |
| | CTTTGTGTCTTGTCTAATGTCCGGTGCACCAATCTGTTCTCGTGTTCGATTCATGTATGTTCGTGTCCAGTCTGTATGAATGAATGTTCTATGTTTTGTGTTGGAT | | |
| | AATAAAGATGGTATAAAAAACTTTATCTGCAAAGCCGAGAGCTGCCACGTGTTTCAGCCAGGAATCAGACACGTGGCGAGAGGGCCCCTGCTGGAAAAACT | | |
| | GTTCGTTTTAGAAAATAAGGGCGAGTGCACAGCCTCTAAGTTTCAGAGTAAAAAAGCTAATAAATGGTTCATGATTAATGTGTTTGACAATGGTAAAGTGTTT | | |
| | TTTATTTTATGATTGTAGCTACAAAAATTATTATTCTCTGATTGGTCTAAATGTAACTGCTTCATTTGGTTCTTTTTTATTGGTAATGTGCTCTAAGTGTTTTCACA | | |
| | ATCAGCTCATAAGTTGTTGGTTAAGATTAATAATTGTTACATTGCTACAGATGGTTAGTGTTAAATTTGATAACTCAAGTTTAGAGTCCTTCCAACACATGGCA | | |
| | TAAGGCAGCCCAAGAGGCTGGGTCTCTAAAGATATTTCTAGTTTAGGTAATAATTAATATGGTTCGTATCCTAAATAGTAAAATTTAAAATAAGATTTAAAGC | | |
| | AATGTCTCTTTATTAAAGCATTAAAGCTTGCTTTAATAGGTATTCATAGGTATTAATTGACATCCAAACTTCGTAATACGATAGTAATGCTTCTAATATTGATTTT | | |
| | AAAGATAATAAATTGTTTTAAACTTGGGTTTTGCTTTCCCAAGGTTATAGGTATTATCCTAACCTTGCACAAAAAACTTAAAAATTATGGTTAAAACTGCTATTG | | |
| | TTCCATTGACTGCAGCTTGCAGTTTGATTTCAAATTTAAGATCTTTAATTCACCTGTATACTGTAATTAAGATAATTACAAGAGTAATCATCTTATGAGAGCGCT | | |
| | CATACAGCTCACTTCATACAAAACTGTGACAGAGTTATCTAGTTATGTTTGTCTTTGTGAATAGATTAGACAAACAGTTTGGTTATAGTTGCTGCCTGGCAGGA | | |
| | AACTGTAGTACAGGCAATTTTTTCACAACACTAAAGTTGTGAAGAACGTTTTAACTGTAAGTCATCTTAAGAAAGAGTATCAAAATTTAGAGGCGTAGACAGT | | |
| | TATATTGTTTCTCTAAAATCGGTCCTTATTACAGGAGGGCCAGGATGCTCAGATTAAAAAGTATTTTACGTTTGAGTCAATGCAGGGCTCTGGGCAGCCCCAG | | |
| | AGCGGCTTGTGGCCTTTCTTTTGTTTTGCAAACAGTGCCTGAGAAAGATTTTTCCCTGTGTTCAAGAAAAATTCTTTTTAACAGTGTTGCAGATCTATTCAGATG | | |
| | TTTAAAATAATGCTTAAATTCAAAGAGTTTTGCTTCTAGTGAACTGTAATCACTAGAAAATTTTGCCTCTAGGTATGGCTAATGTAACTTTACATTATGTAAGAA | | |
| | AAATTTTATTGTTTCTGCTTCTATACAAGAAGCCAAGAGTTTTAATCTTTCAGTGTATATTGTTTCCTAAGTGAAAAGTATTTTATTAACTAATGCTTCTTAAAGT | | |
| | TTACCTTAAATCCTTGCTCTCACCCAAAAGATTCAGAGACAATATCCTTTTATTACTTAGGGTTTTAGTTTACTACAAAAGTTTCTACAAAAAATAAAGCTTTTAT | | |
| | AATTGTTATTAATTGGTAATTAAAAATTGGTTGTGCCCAAAACAATTCTTTGGCCAAAAAAAAAAACATTATTGTAAAGTCATTTTTCTCATCCTCCCAGCCAAT | | |
| | CGTTGGCCCACGTGGGCCCAACTAGCTTCTGTGGGGCAGAGTCTTAAGACACAGTTTCCCCTGTTCCAGCACAGATGATCTAGTTGTGTGCTGTAGATGGTGT | | |
| | TTTAAAATGCTGAACAATCAAACCTTAATTTGTATATTAATAGTCAATGCCATATCTCTGAGCTCACAATTGCTTAAATTGTTCATCCCTCAGATACTATTAATTC | | |
| | TCAAATTTACAATTGCTTATGCATATTTCTAGTTAATAAATAAATTATGCACATGTGACTCTTAATAACTTTACAAGCCTTCTAGTTACAACTGCTCCTTAAGAAA | | |
| | ATTGATTAAAAGTGCAATTAGTCACTGCCCCTTTACAGCCAAGTATTTAAAATGTTTTGTCAACTAGTTATTAATTCAAAAGTTTAGGTATTGTAAAATTTTAAA | | |
| | ACTTTAACTTCTTAAAAGACAAAAAAGAGAGAAATTGTATCCCCGTGCATGCTATTTAGTGCATTCCCATGCACACTATTAAAGTCTTACCTTTATTTTCAAAAT | | |
| | CTAATATTTTAATGTCAAAGAGTTTAATAAATGCTTTATAGTATCTTAAAGGGATCTACTTATTGGCTTATAGATTAATCCTAAAACAGCTACCTTATTAAAAAA | | |
| | GGGAAAAACAGGTTTTCTCCACAGAACGCTGCAGAAGCATATTAATAAATTCGTGTGACGAGCTGGTAGGTAAGTTGACTCATGTCCTGATTAAATTGACTAA | | |
| | AAAACTAAATTAAATTCATGTTTTAGATCCATCCTTACTTGTCATTTTTCCAGTTTAGACTAGCTTCTAGCCTTTTAACTTTATGACAATAGTACATCAGAGACTG | | |
| | TATATTCAGACTTAGTAAAATTAGTCATTTAATAGAGTCATAATGATTTTTCTCCTTTCTTCAGTGTGACCAGCCATTCTAACTCAATCTTAGACTGGTCCTAATA | | |
| | TTCAGTCCAATGTTAGAGATTCCTATATTCTAAATTACCTAGCAAGTTAATTAAAGAGCAATTGGTCACTTAATACCCCCTGACTAACAAATGGAAGGGTCCAG | | |
| | ATCCAGTTCTAATTTGGGGTAGGGACTCAGTTTGTGTTTTTTCACGAGATGAAGATGGAGCGCGGTGGCTGCCAGAGAGATTAATTCGTCAGATGAACACAG | | |
| | ATTCTGACTCTTCTGGTAAGTATCATTCTAAAGACTAAAATTCCTTTTGTGCTTAAAATTCAGCTGAGAGCAACAGCTCTCAAAGGTGTTCTCCAGCTACTCTCT | | |
| | GAACCAGCTCCCGACAGGAGGCCGGAGACTAGCCTCAGCTTTACAATTTGCATTTGAATAAAGTACCTAGACTTCCCGGAAAGAAGTTCTGCTTTCCTACTTT | | |
| | CTCACTGTCTTTCAAGATTTTGTCTTTCAAGCAGGTAAATCAACATTCCCGAGGCGGACCAGTAGATGTGCATCCCCGCCCCCCTAGAGCACACAGGTGGCAG | | |
| | CTGTTACCCCCAGTCTCAGGACATTTCCAGCATGTGGCTTTCAGTCTGAGTTAAAAATTTAGGTTTACCTAGAGGGCTAGAAGAGTAGATTTTTCTATATTAAT | | |
| | AAAGATTGGTTTTTATTTTGATAGACAGGCTTAGCCCCTTAGCTGACCTCTGGCTTTTCACCCTTGCTGTTACTGCAAGGTGTCCTTAGCTCAATAGGCTGTGG | | |
| | AAAAAACAGGGATGAGGAGGAACGACTTCCAGCTCCTATTTTACCCACAAATCGTGGTGTTATTAACGACATAATTCTTGCTTAGGCTTTGCTAATTCTGAGG | | |
| | TTGATAATTCTCCTTTAGGAGCTGCACAGCACTCAGAACTGTGCATACTGGTTTGTGATTGTACAAATTCAGTATGGGCACCGCTTGGTGCAGAGATACTTACT | | |
| | GCAAGGGAAGGTCCGGCTTGACCATTTCTGAGTTTCCTGTGAGATAAACCCGGTTTGAAAGAGGTTGGTACCAAATTTTGGTTAAAAATAAAAAATATTCTCC | | |
| | GGCTCTACCTCGCCTCCCCAAAAGGTACCAAGAGCCACATGTGTGGGTTTTACCCACGGGAGGAATCGGGTCCATGTCCACCCAAGCCAAGGTTAAAAGCCC | | |
| | ACTCATCTACGGATGAGAAAATCATTTGATCACCTCAGTTAAGCGTTGCCTTATTTAACTTAATTAATAGGGGGGAGAGAGATTGGAGACGTACTATTGAAAG | | |
| | GGCAAGCCCTTCACTGCCTCCCACCCAAATAAAAAGGCCAATTGGCCTTGTACTACAAGAGCCGGTCACTCCTTCTCCCTGTTTCCCACCTATCTTCCAAAAATG | | |
| | CTAAGGAATTCAACTTAGTGTTATTTTCACATCGTTCAGTCAAACTTAGCCAGAGTTCCAAACGCCCTACTTAAAATTCAACTAGAAAGTTACCTACCAAGTACT | | |
| | AATTAGCATTATAAAGTCAGAGTCTGCAGCTCCAGGCCTTTCAGTTGTTTACTAGAAAGGACAGTCTTAAGCCAGATACAGTTTACCATAAGAAAAGTTAAAG | | |
| | ATTCCCAGTGAAGCAAGTTTTTTCTTTAGCCCTAGATTCCAGGCAGAACTATTGAGCATAGATAATTTTCCCCCCTCAGGCCAGCTTTTTCTTTTTTTTTTTATTTT | | |
| | GTTAATAACAGGGAGGAGATGTAGTCTCCCCTCCCCTAGCCTGAAACCTGCTTGCTCGGGGTGGAGCTTCCTGCTCATTCGTTCTGCCACGCCCACTGCTGGA | | |
| | ACCTGAGGAGCCACACACGTGCACCTTTCTACTGGACCAGAGATTATTCGGCGGGAATCGGGTCCCCTCCCCCTTCCTTCATAACTGGTGTCGCAACAATAAA | | |
| | ATTTGAGCCTTGATCAGAGTAACTGTCTTGGCTACATTTCTTCTTTTGCCCCGTCTAGATTCCTCTCTTACAGCTCGAGCGGCCTTCTCAGTCGAACCGTTCACG | | |
| | TTGCGAGCTGCTGGCGGCCGCAACA | | |
| ETnI 1 | TGTAGTCTCCCCTCCCCTAGCCTGAAACCTGCTTGCTCAGGGGTGGAGCTTCCTGCTCATTCGTTCTGCCACGCCCACTGCTGGAACCTGCGGAGCCACACACG | 379 | 5497 |
| | TGCACCTTTCTACTGGACCAGAGATTATTCGGCGGGAATCGGGTCCCCTCCCCCTTCCTTCATAACTAGTGTCGCAACAATAAAATTTGAGCCTTGATCAGAGT | | |
| | AACTGTCTTGGCTACATTCTTTTCTCTCGCCACCTAGCCCCTCTTCTCTTCCAGGTTTCCAAAATGCCTTTCCAGGCTAGAACCCAGGTTGTGGTCTGCTGGCCG | | |
| | GACACAACAATTGGCGAACCAACGTGGGACTGAGAAACGGCAAAGGATTTTTGGAAGAGATGCTGCTGGTTCGGAGCTCCATAAAATAAAGGATAAGGGA | | |
| | AATTCATACCGGAGAAGGTATGGGCTAAGCTGAGACAGCGTTAAACCCGAGCGCTGGTTCACTTAGGTTCAGCAGTGAGGAGCTGGGTATCAGGCGGTAGG | | |
| | CCGTAGCTCTCCGAAGCTACATGAGGTGTGAGAAAAGAAAGGGTTTATTAAAAGGAATAGGCGGATTGCCCCAGTTAATAAAAAATGCATCATAAGGGAGG | | |
| | AAAATGTCCCAAAAAGCAGAGAGAAATATCTCTCTGGGCCTTATAGCAGGAGTACTCTGTTCCCTTTTGTGTCTTGTCTAATGTCCGGTGCACCAATCTGTTCT | | |
| | CGTGTTCAATTCATGTATGTTCGTGTCCAGTCTGTATGAATGAATGTTCTATGTTTTGTGTTGGATAATAAAGATGGTATAAAAAACTATCTGCAAAGCCGAGA | | |
| | GCTGCCACGTGTTTCAGCCAGGAATCAGACACGTGGCGAGAGGGCCCCTGCTGGAAAAACTGTTCGTTTTAGGAAATAAGGGCGAGTGCACAGCCTCTAAG | | |
| | TTTCAGAGTAAAAAAGCTAATAAATGGTTCATGATTAATGTGTTTGACAATGGTAAAGTGTTTTTTATTTTTATGGTTGTAGCTACAAAAATTATTATTCTCTGA | | |
| | TTGGTCTAAATGTAACTGCTTCATTTGGTTCTTTTTTATTGGTAATGTGCTCTGAGTGTTTTCACAATCAGCTCATAAGTTGTTGGTTAAGATTAATAATTGTTAC | | |
| | ATTGCTACAGATGGTTAGTGTTAAATTTGATAACTCAAGTTTAGAGTCCTTCCGACACGTGGCATAAAGCAGGCCAAGAGGCTGGGTCTCTAAAGATATTTCT | | |
| | AGTTTAGGTAATAATTAATGTGGTTCGTATCCTAAATAGTAAAATTTAAAATAAGATTTAAAGCAATGTCTCTTTATTAAAGCATTAAAGCTTGCTTTAATAGGT | | |
| | ATTCATAGGTATTAATTGACATCCAAACTTCGTAATATGATAGTAATGCTTCTAATATTGATTTTAAAGATAATAAATTGTTTTAAACTTGGGTTTTGCTTTCCCA | | |
| | AGGTTATAGGTATTATCCTAACCTTGCACAAAAAACTTAAAAATTATGGTTAAAACTGCTATTGTTCCATTGACTGCAGCTTGCAGTTTGATTTCAAATTTAAGA | | |
| | TCTTTAATTCACCTGTATACTGTAATTAAGATAATTACAAGAGTAATCATCTTATGAGAGCGCTCATACAGCTCACTTCATACAAAACTGTGACAGAGTTATCTA | | |
| | GTTATGTTTGTCTTTGTAAATAGATTAGACAAACAGTTTGGTTATAGTTGCTGCCTGGCAGGAAACTGCAGTACGGGCAATTTTTTCACAACACTAAAGTTGTG | | |
| | AAGAACGTTTTAACTGTAAGTCATCGTAAGAAAGAGTATCATAATTTAGAGGCATAGACAGTTATATTGTTTCTCTAGAATCGGTCCTTATTACAGGAGGGCC | | |
| | AAGATGCTCAGATTAAAAAGTATTTTACGTTTGAGTCAATGCAGGGCTCTGGGCAGCCCCAGAGCGGCTTGTGGCCTTTCTTTTGTTTTGCAAACAGTGCCTG | | |
| | AGAAAGATTTTTCCCTGTGTTCAAGAGAAATTCTTTTTAACAGTGTTGCAGATCTATTCAGATGTTTAAAATAATGCTTAAATTCAAAGAGTTTTGCTTCTAGTG | | |
| | AACTGTAATCACTAGAAAATTTTGCCTCTAGGTATGGCTAATGTAACTTTACATTATGTAAGAAAAAATTTTATTGTTTCTGCTTCTATACAAGAAGCCAAGAGT | | |
| | TTTAATCTTTCAGTGTATATTGTTTCCTAAGTGAAAAGTATTTTATTAACTAATGCTTCTTAAAGTTTACCTTAAATCCTTGCTCTCACCCAAAAGATTCAGAGAC | | |
| | AATATCCTTTTATTACTTAGGGTTTTAGTTTACTACAAAAGTTTCTACAAAAAAGAAAGCTTTTATAATTGTTATTAATTGGTAATTAAAAATTGGTTGTGCCCA | | |
| | AAACAATTCTTTGGCCAGAAAAAACATTATTGTAAAGTCATTTTTCTCATCCTCCCAGCCAATCGTTGGCCCATGTGGGCCCAACTAGCTTCTGTGGGGCGGAG | | |
| | TCTTAAGACACAGTTTTCCCTGTTCCAGCACAGATGATCTAGTTGTGTGCTGTAGATGGTGTTTTAAAATGCTGAACAATCAAACCTTAATTTGTATATTAATAG | | |
| | TCAATGCCATATCTCTGAGCTCGCAATTGCTTAAATTGTTCATCCCTCAGATACTATTAATTCTCAAATTTACAATTGCTTATGCATATTTCTAGTTAATAAATAA | | |
| | ATTATGCACATGTGACTCTTAATAACTTTGCAAGCCTTCTAGTTACAACTGCTCCTTAAGAAAATTGATTAAAAGTGCAATTAGTCACTGCCCCTTTACAGCCAT | | |
| | GTATTTAAAATATTTTGTCAACTAGTTATTAATTCAAAAGTTTAGGTATTATAAAATTTTAAAACTTTAACTTCTTAAAAGACAAAAAAGAGAGAAATTGTATCC | | |
| | CCGTGCATGCTATTTAGTGCATTCCCATGCACACTATTAAAGTCTTACCTTTATTTTCAAAATCTAATATTTTAATGTCAAAGAGTTTAATAAATGCTTTATAGTA | | |
| | TCTTAAAGGGATCTACTTATTGGCTTATAGATTAATCCTAAAACAACTACCTTATTAGAAAAGGGAAAAACAGGTTTTCTCCACAGAACGCTGCAGAAGCATA | | |
| | TTAGTAAATTCGTGTGACGAGCTGGTAGGTAAGTTGACTCATGTCCTGATTGAATTGACTAAAAAACTAAATTAAATTCATGTTTTAGATCCATCCTTACTTGT | | |
| | CATTTTTCCAGTTTAGACTAGCTTCTAGCCTTTTAACTTTATGGCAATAGTACATCAGAGACTGTATATTCAGACTTAGTAAAATTAGTCATTTAATAGAGTCAT | | |
| | AATGATTTTTCTCCTTTCTTCAGTGTGACCAGCCATTCTAACTCAATCTCAGACTGGTCTAATATTCAGTCCAATGTTAGAGATTCCTATATTCTAAATTACCTAA | | |
| | CAAAGTTAATTCAGAGCACATCCCCCACTTCCCCTAGATCCCTAACCTCAGAAGGATTGCTGGCCTTACAGCTAGTGGAAAAAGCTATTAAAGAGCAATTGGT | | |
| | CACTTAATACATTGGTAAAATAGAAGGACCCCCTGACTAACGAATAGAAGGGTCCAGATCCAGTTCTAATTTGGGGTAGGGACTCAGTTTGTGTTTTTTCACG | | |
| | AGATGAAGATAGAGCGCAGTGGCTGCCAGATAAATTAATTTGTTAGATGAACACAGATTCTAAATCTTCTAAAGTATCACCTTGAGGACTAAAATTCCTCTTTT | | |
| | GCTTAAAAGTCGGCTTGAGGACTCAGGCTCCGAAAAAAGCTACTCTCTGAGCCAGCTCCTGACAGGAGGCCGGAGACTAGCCTCAGCTTTACAATTTGCATTT | | |
| | AAATAAAGTACCTAGAGTTCCCCAAAAGAAGTTCTGCTTTCCTACTTTCTCACTGTCCGAGATTTTGTCTTTCAAGCAGGTAAATCAACATTCTCGAGGCGGAC | | |
| | CAGCGGATGTGCATCCCCGCCCCCCTAGCGCACACAGGTGGCTGCTGTTATCTCCTTTCCAAGGACATTTCCAGCATGTGGCTTTCAGTCTGAGTTAAAAATTA | | |
| | GGTTTACCAAGAGGGCTAGAAAAGTAGATATTTCTATATTAATAAAGATTGGTTTTTATTTTGATAGACAGGCTTAGTCCCTTAGCTGACCTCTGGCTTTTCAC | | |
| | CCTTGCTGTTACTGCAAGGTGTCCTTAGGCTGTGGAAAAAACAGGGATGAGGAGAAACGACTTCCAGCTCCTATTTTAGCCACAAATCGTGGTGTTACTAATG | | |
| | ACATAATTCTTGCTTAGGCTTTGCTAATTCTGAGGTTGATAATTCTCCTTTAGGAGCTGCACAGCACTCAGAAATGTGCATACTGGTTTGTGATTGTACAAATTC | | |
| | AGTATGGGCATCGCTTGGTGCAGAGGTACTGCAAGGGAAGGTCCGGCTTGACCATTTCTGAGTTTCCTGTGAGATAAACCCGGTTTAAAAGAGGTTGGTATC | | |
| | ATATTTTGGTTAAAAATCAAAAATATTTTCCGGCTCTGCCTCGCCTCCCCAAAAGATACCCAGAGTCACATGTGTGGGTCTTATCAATACCCACGGGAGGAATC | | |
| | GGGTCCATGTCCACCCAAGCCAAGGTTAAAAGCCCACTCATCTACGGATGAGAAAATCATTTGATCACCTCAGTTAAGCGTTGCCTTATTTAACTTAATCAATA | | |
| | GGGGGGAGAGAGATTGGAGACGTACTATTGAAAGGGCAAGCCCTTCACTGCCTCCCACCCAAATAAAAAAGCCAATTGGCCTTGTACTACAAGAGCCGGTC | | |
| | GAACTCCTTCTCCCTGTTTCCCACCTATCTTCCAAAAAAGCGGAGAAATATCAACTTAGTGTTATTATAGTGTATTTCACATTTGTTCAGTCAAACTTAGCCAGA | | |
| | GTTCCAACGCCCTACTTAAAATTCAACTAGAAAGTTACCTACCAAGTACTAATTAGCATTATAAAGTCAGAGCCTACAGCTCCAGGCTTTTCAGTTAGTTGTTC | | |
| | ACTAAGATAAGAAAGGACAGTCTTAGCCTTATACAGTTTACCATAAGAAAAGTTAAGGAATCCCATGAAAGCAAGTTTTTTCTTTAGCCCTAGATTCCAGGCA | | |
| | GAACTATTGAGCATAGATAATTTTTCCCCCCTCAGGCCAGCTTTTTTTTTTTTAAATTTTGTTAATAAAAGGGAGGAGATGTAGTCTCCCCTCCCCTAGCCTGA | | |
| | AACCTGCTTGCTCAGGGGTGGAGCTTCCTGCTCATTCGTTCTGCCACGCCCACTGCTGGAACCTGCGGAGCCACACACGTGCACCTTTCTACTGGACCAGAGA | | |
| | TTATTCGGCGGGAATCGGGTCCCCTCCCCCTTCCTTCATAACTAGTGTCGCAACAATAAAATTTGAGCCTTGATCAGAGTAACTGTCTTGGCTACATTCTTTTCT | | |
| | CTCGCCACCTAGCCCCTCTTCTCTTCCAGGTTTCCAAAATGCCTTTCCAGGCTAGAACCCAGGTTGTGGTCTGCTGGCCGGACACAACA | | |
| IAP- | TGTGTTGGGAGCCGCCCCCACATTCGCCGTTACAAGATGGCGCTGACATCCTGTGTTCTAAGTGGTAAACAAATAATCTGCGCATGTGCCAAGGGTATCTTAT | 380 | 7072 |
| RP23- | GACTACTTGTGCTCTGCCTTCCCCGTGACGTCAACTCGGCCGATGGGCTGCAGCCAATCAAGGAGTGACACGTCCGAGGCGAAGGAGAATGCTCCTTAAGAG | | |
| 231P12 | GGACGGGGTTTTCGTTTTCTCTCTCTCTTGCTTCTTGCTCTCTTTTCCTGAAGATGTAAGAATAAAGCTTTGCCGCAGAAGATTCTGGTCTGTGGTGTTCTTCCT | | |
| | GGCCGGTCGTGAGAACGCGTCTAATAACAATTGGTGCCGAATTCCGGGACGAGAAAATCCGGGACGAGAAAAAACTCCGGACTGGCGCAGGAGGGATACT | | |
| | TCATTCCAGAACCAGAACTGCGAATCAAGGTTATAAGGTTCCCGTAACACAGACTGTTGAGAAGGATTCAACTGCCGAATTCAGAACTCATCAGCTGGGGAA | | |
| | CGACGGTGATAAAGGTTCCCGTAAAGCAGACTGTTAAGAAGGATTCAACTGTATGAATTCAGAACTTTTCAGCTGGGGAACGAGAGTACCAGTGAGTATGTT | | |
| | TGGCCTTGAATTTTTTCTGGTGTTAGGAGCCCTTTTGTTCTTTTTCACATGCTATCAAGTGATTAAGATAGGGCTGAAAATTCTAGAGGAAATTCAGGACAAGC | | |
| | TATCAGAAGTAAAGCGGGAAGAAAGAGTAGGAACAAAGAGAAAATATGGTTCACAAAATAAGTATACAGGCCTTTCCAAGGGTCTTGAACCCGAGGAAAA | | |
| | GTTAAGGTTAGGTAGGAATACCTGGAGAGAGATTAGAAGAAAAAGAGGAAAAAGGGAGAAGAAAAAAGATCGATTAGCGGAGGTCTCTAGGAGATACTC | | |
| | GTCACTAGATGAGCTCAGGAAGCCAGCTCTTAGTAGCTCTGAAGCAGATGAAGAATCCTCCTCTGAGGAAACAGACTGGGAGGAAGAAGCAGCCCATTACC | | |
| | AGCCAGCTAATCGGTCAAGAAAAAAGCCAAAAGCGGCTGGCGAAGGCCAGTTTGCTAATTGGCCTCAGGGCAATCGGCTACCAGGTGCACTCCCGCCCTAT | | |
| | GCGGAGTCCCCGCCCTGCGTAGTGCGTCAGCCCGTAGTGCGTCAGCAATGCGCAGAGAGGCAGTGCGCAGACTCATTCATTCCCCGAGAGGAACAAAGGAA | | |
| | AATAGAACAGGCATTTCCAGTCTTTGAAGGAGCCGAGGGTGGGCGTGTCCACGCTCCGGTAGAATACGTACAGATTAAGGAAATTGCCGAGTCGGTTCGTA | | |
| | AATACGGAACCAATGCTAATTTTACCTTGGTGCAGTTAGACAGGCTCGCTGGCATGGCACTAACTCCTGCTGACTGGCAAACGGTTGTAAAAGCCGCTCTCCC | | |
| | TAGTATGGGCAAATATATGGAATGGAAAGCGCTTTGGCACGAAGCTGCACAGGCGCAGGCCCGAGCAAACGCAGCTGCTTTGACTCCAGAGCAGAGAGATT | | |
| | GGACTTTTGACTTGTTAACGGGTCAGGGAGCTTATTCTGCTGATCAGACAAACTACCATTGGGGAGCTTATGCCCAGATTTCTTCCACGGCTATTAGGGCCTG | | |
| | GAAGGCGCTCTCTCGAGCAGGTGAAACCACTGGTCAGTTAACAAAAATAATCCAGGGACCTCAGGAATCCTTCTCAGATTTTGTGGCCAGAATGACAGAGGC | | |
| | AGCAGAGCGTATTTTTGGAGAGTCAGAGCAAGCTGCGCCTCTCATAGAACAGCTAATCTACGAGCAAGCCACAAAGGAGTGCCGAGCGGCCATAGCCCCAA | | |
| | GAAAGAACAAAGGCTTACAAGACTGGCTCAGGGTCTGTCGAGAGCTTGGGGGACCCCTCAGCAATGCAGGTTTAGCGGCTGCCATCCTTCAATCCCAAAACC | | |
| | GCTCCATGGGCAGAAATGATCAGAGGACATGTTTTAACTGCGGAAAGCCTGGGCATTTTAAGAAAGATTGCAGAGCTCCAGATAAACAGGGAGGGACTCTC | | |
| | ACTCTTTGCTCTAAGTGTGGCAAGGGTTATCATAGAGCTGACCAGTGTCGCTCTGTGAGGGATATAAAGGGCAGAATTCTTCCCCCACCTGATAGTCAATCAA | | |
| | CTGATGTGCCAAAAAACGGGTCATCGGGCCCTCGGTCCCAGGGCCCTCAAAGATATGGGAACCGGTTTGTCAGGACCCAGGGGGCAGTCAGAGAGGCGAC | | |
| | CCAGGAAGACCCACAAGGGTGGACCTGCGTGCCGCCTCCGACTTCCTACTAATGCCTCAAATGAGTATTCAGCCGGTGCCGGTGGAGCCTATACCATCCTTGC | | |
| | CCCCGGGAACCATGGGCCTTATTCTCGGCCGAGGTTCACTCACCTTGCAGGGCTTAGTAGTCCACCCTGGAGTTATGGATTGTCAACATTCCCCTGAAATACA | | |
| | GGTCCTGTGCTCAAGCCCTAAAGGCGTTTTTTCTATTAGTAAAGGAGATAGGATAGCTCAGCTGCTGCTCCTCCCTGATAATACCAGGGAGAAATCTGCAGGA | | |
| | CCTGAGATAAAGAAAATGGGCTCCTCAGGAAATGATTCTGCCTATTTGGTTGTATCTTTAAATGATAGACCTAAGCTCCGCCTTAAGATTAATGGAAAAGAGT | | |
| | TTGAAGGCATCCTTGATACCGGAGCAGATAAAAGTATAATCTCTACACATTGGTGGCCCAAAGCATGGCCCACCACAGAGTCATCTCATTCATTACAGGGCCT | | |
| | AGGATATCAATCATGTCCCACTATAAGCTCCGTTGCCTTGACGTGGGAATCCTCTGAAGGGCAGCAAGGGAAATTCATACCTTATGTGCTCCCACTCCCGGTT | | |
| | AACCTCTGGGGAAGGGATATTATGCAGCATTTGGGCCTTATTTTGTCCAATGAAAACGCCCCATCAGGAGGGTATTCAGCTAAAGCAAAAAATATCATGGCA | | |
| | AAGATGGGTTATAAAGAAGGAAAAGGGTTAGGACATCAAGAACAGGGAAGGATAGAGCCCATCTCACCTAATGGAAACCAAGACAGACAGGGTCTGGGTT | | |
| | TTCCATAGCGGCCATTGGGGCAGCACGACCCATACCATGGAAAACAGGGGACCCAGTGTGGGTTCCTCAATGGCACCTATCCTCTGAAAAACTAGAAGCTGT | | |
| | GATTCAACTGGTAGAGGAACAATTAAAACTAGGCCATATTGAACCCTCTACCTCACCTTGGAATACTCCAATTTTTGTAATTAAGAAAAAGTCAGGAAAGTGG | | |
| | AGACTGCTCCATGACCTCAGAGCCATTAATGAGCAAATGAACTTATTTGGCCCAGTACAGAGGGGTCTCCCTGTACTTTCCGCCTTACCACGTGGCTGGAATT | | |
| | TAATTATTATAGATATTAAAGATTGTTTCTTTTCTATACCTTTGTGTCCAAGAGATAGGCCCAGATTTGCCTTTACCATCCCCTCTATTAATCACATGGAACCTGA | | |
| | TAAGAGGTATCAATGGAAGGTCTTACCACAGGGAATGTCCAATAGTCCTACAATGTGCCAACTTTATGTGCAAGAAGCTCTTTTGCCAGTGAGGAAACAGTTC | | |
| | CCCTCTTTAATTTTGCTCCTTTACATGGATGACATCCTCCTGTGCCATAAAGACCTTACCATGCTACAAAAGGCATATCCTTTTCTACTTAAAACTTTAAGTCAGT | | |
| | GGGGTTTACAGATAGCCACAGAAAAGGTCCAAATTTCTGATACAGGACAATTCTTGGGCTCTGTGGTGTCCCCAGATAAGATTGTGCCCCAAAAGGTAGAGA | | |
| | TAAGAAGAGATCACCTCCATACCTTAAATGATTTTCAAAAGCTGTTGGGAGATATTAATTGGCTCAGACCCTTTTTAAAGATTCCTTCTGCTGAATTAAGGCCT | | |
| | TTGTTTAGTATTTTAGAAGGAGATCCTCATATCTCCTCCCCTAGGACTCTTACTCTAGCTGCTAACCAGGCCTTACAAAAAGTGGAAAAAGCCTTACAGAATGC | | |
| | ACAATTACAACGTATTGAGGATTCGCAGCCTTTCAGTTTGTGTGTCTTTAAGACAGCACAATTGCCAACTGCAGTTTTGTGGCAGAATGGGCCATTGTTGTGG | | |
| | ATCCATCCAAACGTATCCCCAGCTAAAATAATAGATTGGTATCCTGATGCAATTGCACAGCTTGCCCTTAAAGGCCTAAAAGCAGCAATCACCCACTTTGGGC | | |
| | AAAGTCCATATCTTTTAATTGTACCTTATACCGCTGCACAGGTTCAAACCTTGGCAGCCGCATCTAATGATTGGGCAGTTTTAGTTACCTCCTTTTCAGGAAAAA | | |
| | TAGATAACCATTATCCAAAACATCCAATCTTACAGTTTGCCCAAAATCAATCTGTTGTGTTTCCACAAATAACAGTAAGAAACCCACTTAAAAATGGGATTGTG | | |
| | GTATATACTGATGGATCAAAAACTGGCATAGGTGCCTATGTGGCTAATGGTAAAGTGGTATCCAAACAATATAATGAAAATTCACCTCAAGTGGTAGAATGTT | | |
| | TAGTGGTCTTAGAAGTTTTAAAAACCTTTTTAAAACCCCTTAATATTGTGTCAGATTCCTATTATGTGGTTAATGCAGTAAATCTTTTAGAAGTGGCTGGAGTG | | |
| | ATTAAGCCTTCCAGTAGAGTTGCCAATATTTTTCAGCAGATACAATTAGTTTTGTTATCTAGAAGATCTCCTGTTTATATTACTCATGTTAGAGCCCATTCAGGC | | |
| | CTACCTGGCCCCATGGCTCTGGGAAATGATTTGGCAGATAAGGCCACTAAAGTGGTGGCTGCTGCCCTATCATCCCCGGTAGAGGCTGCAAGAAATTTTCAT | | |
| | AACAATTTTCATGTGACGGCTGAAACATTACGCAGTCGTTTCTCCTTGACAAGAAAAGAAGCCCGTGACATTGTTACTCAATGTCAAAGCTGCTGTGAGTTCTT | | |
| | GCCAGTTCCTCATGTGGGAATTAACCCACGCGGTATTCGACCTCTACAGGTCTGGCAAATGGATGTTACACATGTTTCTTCCTTTGGAAAACTTCAATATCTCC | | |
| | ATGTGTCCATTGACACATGTTCTGGCATCATGTTTGCTTCTCCGTTAACTGGAGAAAAAGCCTCACATGTGATTCAACATTGTCTTGAGGCATGGAGTGCTTGG | | |
| | GGGAAACCCAGACTCCTTAAGACTGATAATGGACCAGCTTATACGTCTCAAAAATTCCAACAGTTCTGCCGTCAGATGGACGTGACCCACCTGACTGGACTTC | | |
| | CATACAACCCTCAAGGACAGGGTATTGTTGAGCGTGCGCATCGCACCCTCAAAGCCTATCTTATAAAACAGAAGAGGGGAACTTTTGAGGAGACTGTACCCC | | |
| | GAGCACCAAGAGTGTCGGTGTCTTTGGCACTCTTTACACTTAATTTTTTAAATATTGATGCTCATGGCCATACTGCGGCTGAACGTCATTGTTCAGAGCCAGAT | | |
| | AGGCCCAATGAGATGGTTAAATGGAAAAATGTCCTTGATAATAAATGGTATGGCCCGGATCCTATCTTGATAAGATCCAGGGGAGCTATCTGTGTTTTCCCAC | | |
| | AGAATGAAGACAACCCATTTTGGGTACCAGAAAGACTCACCCGAAAAATCCAGACTGACCAAGGGAATACTAATGTCCCTCGTCTTGGTGATGTCCAGGGCG | | |
| | TCAATAATAAAGAGAGAGCAGCGTTGGGGGATAATGTCGACATTTCCACTCCCAATGACGGTGATGTATAATGCTCAAGTATTCTCCTGCTTTTTTACCACTAA | | |
| | CTAGGAACTGGGTTTGGCCTTAATTCAGACAGCCTTGGCTCTGTCTGGACAGGTCCAGATGACTGACACCATTAACACTTTGTCAGCCTCAGTGACTACAGTC | | |
| | ATAGATGAACAGGCCTCAGCTAATGTCAAGATACAGAGAGGTCTCATGCTGGTTAATCAACTCATAGATCTTGTCCAGATACAACTAGATGTATTATGACAAA | | |
| | TAACTCAGCAGGGATGTGAACAAAAGTTTCCGGGATTGTGTGTTATTTCCATTCAGTATGTTAAATTTACTAGGGCAGCTAATTTGTCAAAAAGTCTTTTTCAG | | |
| | TATATGTTACAGAATTGGATGGCTGAATTTGAACAGACCCTTCGAGGCTTGCCATCATTCAGGTCAACTCCACGCGCTTGGACCTGTCCCTGACCAAAGGATT | | |
| | ACCCAATTGGATCTCCTCAGCATTTTCTTTCTTTAAAAAATGGGTGGGATTAATATTATTTGGAGATACACTTTGCTGTGGATTAGTGTTGCTTCTTTGATTGGT | | |
| | CTGTAAGCTTAAGGCCTAAACTAGGAGAGACAAGGTGGTTATTGCCCAGGCGCTTGCAGGACTAGAACATGGAGCTTCCCCTGATATATCTATGCTTAGGCA | | |
| | ATAGGTCGCTGGCCACTCAGCTCTTATATCTCACGAGGCTAGTCTCATTGCACGAGATAGAGTGAGTGTGCTTCAGCAGCCCGAGAGAGTTGCAAGGCTAAG | | |
| | CACTGCAATGGAAAGGCTCTGCGGCATATATGAGCCTATTCTAGGGAGACATGTCATCTTTCATGAAGGTTCAGTGTCCTAGTTCCCTTCCCCCAGGCAAAAC | | |
| | GACACGGGAGCAGGTCAGGGTTGCTCTGGGTAAAAGCCTGTAAGCCTAAGAGCTAATCCTGTACATGGCTCCTTTACCTACACACTGGGGATTTGACCTCTAT | | |
| | CTCCACTCTCATTAATATGGGTGGCCTATTTGCTCTTATTAAAAGAAAAAGGGGGAGATGTTGGGAGCCGCCCCCACATTCGCCGTTACAAGATGGCGCTGAC | | |
| | ATCCTGTGTTCTAAGTGGTAAACAAATAATCTGCGCATGTGCCAAGGGTATCTTATGACTACTTGTGCTCTGCCTTCCCCGTGACGTCAACTCGGCCGATGGGC | | |
| | TGCAGCCAATCAAGGAGTGACACGTCCGAGGCGAAGGAGAATGCTCCTTAAGAGGGACGGGGTTTTCGTTTTCTCTCTCTCTTGCTTCTTGCTCTCTTTTCCTG | | |
| | AAGATGTAAGAATAAAGCTTTGCCGCAGAAGATTCTGGTCTGTGGTGTTCTTCCTGGCCGGTCGTGAGAACGCGTCTAATAACA | | |
| IAP- | TGTTGGGAGCCGCGCCCACATTCGCCGTTACAAGATGGCGCTGACAGCTGTGTTCTAAGTGGTAAACAAATAATCTGCGCATGTGCCAAGGGTATCTTATGA | 381 | 7086 |
| RP23- | CTACTTGTGCTCTGCCTTCCCCGTGACGTCAACTCGGCCGATGGGCTGCAGCCAATCAGGGAGTGACACGTCCGAGGCGAAGGAGAATGCTCCTTAAGAGG | | |
| 262J21 | GACGGGGTTTCGTTTTCTCTCTCTCTTGCTTCTTGCTCTCTTGCTTCTTACACGCTTGCTCCTGAAGATGTAAGAAATAAAGCTTTGCCGCAGAAGATTCTGGTC | | |
| | TGTGGTGTTCTTCCTGGCCGGTCGTGAGAACGCGTCTAATAACAATTGGTGCCGAATTCCGGGACGAGAAAAAACTCGGGACTGGCGCAAGGAAGATCCCT | | |
| | CATTCCAGAACCAGAACTGCGGGTCGCGGTAATAAAGGTTCCCGTAAAGCAGACTGTTAAGAAGGATTCAACTGTATGAATTCAGAACTTTTCAGCTGGGGA | | |
| | ACGAGAGTACCAGTGAGTACAGCTTTACGAGGTAAGTCCGATCTTGAACTTTCTAACGAAATTCAAGACAGTCTATCAGAAGTAAAGTGGAATATGTTTGGC | | |
| | CTTGAATTTTTTCTGGTGTTAGGAGCCCTTTTGTTCCTTTTCACATGTTATCAAGTGATTAAGATAGGGCTGAAAATTCTAGAGGAAATTCAGGACAAGCTATC | | |
| | AGAAGTAAAGCGGGGAGAGAGAGTAGGAGCAAAGAGGAAATATGGTACACAAAATAAGTATACAGGCCTTTCCAAGGGTCTTGAACCCGAGGAAAAGTTA | | |
| | AGGTTAGGTAGGAATACCTGGAGAGAGATTAGAAGAAAAAGAGGAAAAAGGGAAAAGAAGAAAGATCAATTAGCGGAGGTCTCTAGGAGATACTCGTCA | | |
| | CTAGATGAGCTCAGGAAGCCAGCTCTTAGTAGTTCTGAAGCAGGTGAAGAATCCTCCTCTGAGGAAACAGACTGGGAGGAAGAAGCAGCCCATTACCAGCC | | |
| | AGCTAATTGGTCAAGAAAAAAGCCAAAAGCGGCTGGCGAAGGCCAGTTTGCTGATTGGCCTCAGGGCAGTCGGCTTCAAGGTCCGCCCTATGCGGAGTCCC | | |
| | CGCCCTGCGTAGTGCGTCAGCAATGCGCAGAGAGATGCGCAGAGAGGCAGTGCGCAGAGAGGCAGTGCGCAGACTCATTCATTCCCAGAGAGGAACAAAG | | |
| | GAAAATACAACAGGCATTTCCGGTCTTTGAAGGAGCCGAGGGTGGGCGTGTCCACGCTCCGGTAGAATACTTACAAATTAAAGAAATTGCCGAGTCGGTTCG | | |
| | TAAATACGGAACCAATGCTAATTTTACCTTGGTGCAGTTAGACAGGCTCGCTGGCATGGCACTAACTCCTGCTGACTGGCAAACGGTTGTAAAAGCCGCTCTC | | |
| | CCTAGTATGGGCAAATATATGGAATGGAGAGCGCTTTGGCATGAAGCTGCACAAGCGCAGGCCCGAGCAAACGCAGCTGCTTTGACTCCAGAGCAGAGAGA | | |
| | TTGGACTTTTGACTTGTTAACGGGTCAGGGAGCTTATTCTGCTGATCAGACAAACTACCATTGGGGAGCTTATGCCCAGATTTCTTCCACGGCTATTAGGGCCT | | |
| | GGAAGGCGCTCTCTCGAGCAGGTGAAACCACTGGTCAGTTAACAAAGATAATCCAGGGACCTCAGGAATCCTTCTCAGATTTTGTGGCCAGAATGACAGAGG | | |
| | CAGCAGAGCGTATTTTTGGAGAGTCAGAGCAAGCTGCGCCTCTGATAGAACAGCTAATCTATGAGCAAGCCACAAAGGAGTGCCGAGCGGCCATAGCCCCA | | |
| | AGAAAGAACAAAGGCTTACAAGACTGGCTCAGGGTCTGTCGAGAGCTTGGGGGACCTCTCACCAATGCAGGCTTAGCGGCCGCCATCCTCCAATCTCAGAAC | | |
| | CGCTCCATGAGCAGAAATGATCAGAGGACATGTTTTAATTGCGGAAAGCCTGGGCATTTTAAGAAAGATTGCAGAGCTCCAGATAAACAGGGAGGGACTCT | | |
| | CACTCTTTGCTCTAAGTGTGGCAAGGGTTATCATAGAGCTGACCAGTGTCGCTCTGTGAGGGATATAAAGGGCAGAATTCTTCCCCCACCTGATAGTCAATCA | | |
| | ACTGATGTGCCAAAAAACGGGTCATCGGGCCCTCGGTCCCAGGGCCCTCAAAGATATGGGAACCGGTTTGTCAGGACCCAGGAAGCAGTCAGAGAGGCGAC | | |
| | CCAGGAAGACCCACAAGGGTGGACCTGCGTGCCGCCTCCGACTTCCTATTAATGCCTCAAATGAGTATTCAGCCGGTGCCAGTGGAGCCTATACCATCCTTGC | | |
| | CCCCGGGAACCATGGGCCTTATTCTCGGCCGGGGTTCACTCACCTTGCAGGGCTTAGTAGTCCACCCTGGAGTTATGGATTGTCAACATTCCCCTGAAATACA | | |
| | GGTCCTGTGCTCAAGCCCTAAAGGCGTTTTTTCTATTAGTAAAGGAGATAGGATAGCTCAGCTGCTGCTCCTCCCTGATAATACCAGGGAAAAATCTGCAGGA | | |
| | CCTGAGATAAAGAAAATGGGCTCCTCAGGAAATGATTCTGCCTATTTGGTTGTATCTTTAAATGATAGACCTAAGCTCCGCCTTAAGATTAATGGAAAAGAGT | | |
| | TTGAAGGCATCCTTGATACCGGAGCAGATAAAAGTATAATTTCTACACATTGGTGGCCCAAAGCATGGCCCACCACAGAGTCATCTCATTCATTACAGGGCCT | | |
| | AGGTTATCAATCATGTCCCACTATAAGCTCCATTGCCTTGACGTGGGAATCCTCTGAAGGGCAGCAAGGGAAATTCATACCTTATGTGCTCCCACTCCCGGTTA | | |
| | ACCTCTGGGGAAGGGATATTATGCAGCATTTGGGCCTTATTTTGTCCAATGAAAACGCCCCATCGGGAGGGTATTCAACTAAAGCAAAAAATATCATGGCAA | | |
| | AGATGGGTTATAAAGAAGGAAAAGGGTTAGGACATCAAGAACAGGGAAGGATAGAGCCCATCTCACCTAATGGAAACCAAGACAGACAGGGTCTGGGTTT | | |
| | TTCCTTAGCGGCCATTGGGGCAGCACGACCCATACCATGGAAAACAGGGGACCCAGTGTGGGTTCCTCAATGGCACCTATCCTCTGAAAAACTAGAAGCTGT | | |
| | GATTCAACTGGTAGAGGAACAATTAAAATTAGGCCATATTGAACCCTCTACCTCACCTTGGAATACTCCAATTTTTGTAATTAAGAAAAAGTCAGGAAAGTGG | | |
| | AGACTGCTCCATGACCTCAGAGCCATTAATGAGCAAATGAACTTATTTGGCCCAGTACAGAGGGGTCTCCCTGTACTTTCCGCCTTACCACGTGGCTGGAATT | | |
| | TAATTATTATAGATATTAAAGATTGTTTCTTTTCTATACCTTTGTGTCCAAGGGATAGGCCCAGATTTGCCTTTACCATCCCCTCTATTAATCACATGGAACCTGA | | |
| | TAAGAGGTATCAATGGAAGGTCTTACCACAGGGAATGTCCAATAGTCCTACAATGTGCCAACTTTATGTGCAAGAAGCTCTTTTGCCAGTGAGGGAACAATTC | | |
| | CCCTCTTTAATTTTGCTCCTTTACATGGATGACATCCTCCTGTGCCATAAAGACCTTACCATGCTTCAAAAGGCATATCCTTTTCTACTTAAAACTTTAAGTCAGT | | |
| | GGGGTTTACAGATAGCCACAGAAAAGGTCCAAATTTCTGATACAGGACAATTCTTGGGCTCTGTGGTGTCCCCAGATAAGATTGTGCCCCAAAAGGTAGAGA | | |
| | TAAGAAGAGATCACCTCCATACCTTAAATGATTTTCAAAAGCTGTTGGGAGATATTAATTGGCTCAGACCTTTTTTAAAGATTCCTTCCGCTGAGTTAAGGCCT | | |
| | TTGTTTAGTATTTTAGAAGGAGATCCTCATATCTCCTCCCCTAGGACTCTTACTCTAGCTGCTAACCAGGCCTTACAAAAAGTGGAAAAGGCCTTACAGAATGC | | |
| | ACAATTACAACGTATTGAGGATTCGCAACCTTTCAGTTTGTGTGTCTTTAAGACAGCACAATTGCCAACTGCAGTTTTGTGGCAGAATGGGCCATTGTTGTGG | | |
| | ATCCATCCAAACGTATCCCCAGCTAAAATAATAGATTGGTATCCTGATGCAATTGCACAGCTTGCCCTTAAAGGTCTAAAAGCAGCAATCACCCACTTTGGGCA | | |
| | AAGTCCATATCTTTTAATTGTACCTTATACCGCTGCACAGGTTCAAACCTTGGCAGCCACATCTAATGATTGGGCAGTTTTAGTTACCTCCTTTTCAGGAAAAAT | | |
| | AGATAACCATTATCCAAAACATCCAATCTTACAGTTTGCCCAAAATCAATCTGTTGTGTTTCCACAAATAACAGTAAGAAACCCACTTAAAAATGGGATTGTGG | | |
| | TATATACTGATGGATCAAAAACTGGCATAGGTGCCTATGTGGCTAATGGTAAAGTGGTATCCAAACAATATAATGAAAATTCACCTCAAGTGGTAGAATGTTT | | |
| | AGTGGTCTTAGAAGTTTTAAAAACCTTTTTAGAACCCCTTAATATTGTGTCAGATTCCTGTTATGTGGTAAATGCAGTAAATCTTTTAGAAGTGGCTGGAGTGA | | |
| | TTAAGCCTTCCAGTAGAGTTGCCAATATTTTTCAGCAGATACAATTAGTTTTGTTATCTAGAAGATCTCCTGTTTATATTACTCATGTTAGAGCCCATTCAGGCC | | |
| | TACCTGGCCCCATGGCTCTGGGAAATGATTTGGCAGATAAGGCCACTAAAGTGGTGGCTGCTGCCCTATCATCCCCGGTAGAGGCTGCAAGAAATTTTCATA | | |
| | ACAATTTTCATGTGACGGCTGAAACATTACGCAGTCGTTTCTCCTTGACAAGAAAAGAAGCCCGTGACATTGTTACTCAATGTCAAAGCTGCTGTGAGTTCTTG | | |
| | CCAGTTCCTCATGTGGGAATTAACCCACGCGGTATTCGACCTCTACAGGTCTGGCAAATGGATGTTACACATGTTTCTTCCTTTGGAAAACTTCAATATCTCCAT | | |
| | GTGTCCATTGACACATGTTCTGGCATCATGTTTGCTTCTCCGTTAACCGGAGAAAAAGCCTCACATGTGATTCAACATTGTCTTGAGGCATGGAGTGCTTGGG | | |
| | GGAAACCCAGACTCCTTAAGACTGATAATGGACCAGCTTATACGTCTCAAAAATTCCAACAGTTCTGCCGTCAGATGGACGTAACCCACCTGACTGGACTTCC | | |
| | GTACAACCCTCAAGGACAGGGTATTGTTGAGCGTGCGCATCGCACCCTCAAAGCCTATCTTATAAAACAGAAGAGGGGAACTTTTGAGGAGACTGTACCCCG | | |
| | AGCACCAAGAGTGTCGGTGTCTTTGGCACTCTTTACACTCAATTTTTTAAATATTGATGTTCATGGCCATACTGCGGCTGAACGTCATTGTTCAGAGCCAGATA | | |
| | GGCCCAATGAGATGGTTAAATGGAAAAATGTCCTTGATAATAAATGGTATGGCCCGGATCCTATCTTGATAAGATCCAGGGGAGCTGTCTGTGTTTTCCCACA | | |
| | GAATGAAGACAACCCATTTTGGGTACCAGAAAGACTCACCCGAAAAATCCAGACTGACCAAGGGAATACTAATGTCCCTCGTCTTGGTGATGTCCAGGGCGT | | |
| | CAATAATAAAGAGAGAGCAGCGTTGGGGGATAATGTCGACATTTCCACTCCCAATGACGGTGATGTATAATGCTCAAGTATTCTCCTGCTTTTTTACCACTAAC | | |
| | TAGGAACTGGGTTTAGCCTTAATTCAGACAGCCTTAGCTCTGTCTGGACAGGTCCAGACGACTGACACCATTAACACTTTGTCAGCCTCAGTGACTACAGTCA | | |
| | TAGATAAACAGGCCTCAGCTAATGTCAAGATACAGGGAGGTCTCATGCTGGTTAATCAACTCATAGATCTTGTCCAGATACAACTAGATGTATTATGACAAAT | | |
| | AACTCAGCAGGGATGTGAACAAAAGTTTCCGGGATTGTGTGTTATTTCCATTCAGTATGTTAAATTTACTAGGACAGCTAATTTGTCAAAAAGTCTTTTTCAGT | | |
| | ATATGTTACAGAATTGGATGGCTGAATTTGAACAGATCCTTCGGGAATTGAGACTTCAGGTCAACTCCACGCGCTTGGACCTGTCGCTGACCAAAGGATTACC | | |
| | CAATTGGATCTCCTCAGCATTTTCTTTCTTTAAAAAATGGGTGGGATTAATATTATTTGGAGATACACTTTGCTGTGGATTAGTGTTGCTTCTTTGATTGGTCTG | | |
| | TAAGCTTAAGGCCCAAACTAGGAGAGACAAGGTGGTTATTGCCCAGGCGCTTGCAGGACTAGAACATGGAGCTTCCCCTGATATATCTATGCTTAGGCAATA | | |
| | GGTCGCTGGCCACTCAGCTCTTATATCCCATGAGGCTAGACTCATTGCACGGGATAGAGTGAGTGTGCTTCAGCAGCCCGAGAGAGTTGCAAGGCTAAGCAC | | |
| | TGCAATGGAAAGGCTCTGCGGCATATATGAGCCTATTCTAGGGAGACATGTCATCTTTCATGAAGGTTCAGTGTCCTAGTTCCCTTCCCCCAGGCAAAACGAC | | |
| | ACGGGAGCAGGTCAGGGTTGCTCTGGGTAAAAGCCTGTGAGCCTAAGAGCTAATCCTGTACATGGCTCCTTTACCTACACACTGGGGATTTGACCTCTATCTC | | |
| | CACTCTCATTAATATGGGTGGCCTATTTGCTCTTATTAAAAGGATAGGGGGAGATGTTGGGAGCCGCGCCCACATTCGCCGTTACAAGATGGCGCTGACAGC | | |
| | TGTGTTCTAAGTGGTAAACAAATAATCTGCGCATGTGCCAAGGGTATCTTATGACTACTTGTGCTCTGCCTTCCCCGTGACGTCAACTCGGCCGATGGGCTGC | | |
| | AGCCAATCAGGGAGTGACACGTCCGAGGCGAAGGAGAATGCTCCTTAAGAGGGACGGGGTTTCGTTTTCTCTCTCTCTTGCTTCTTGCTCTCTTGCTTCTTACA | | |
| | CGCTTGCTCCTGAAGATGTAAGAAATAAAGCTTTGCCGCAGAAGATTCTGGTCTGTGGTGTTCTTCCTGGCCGGTCGTGAGAACGCGTCTAATAACA | | |
| IAP- | TGTGGGAAGCCGCCCCCACATTCGCCGTCACAAGATGGCGCTGACATCCTGTGTTCTAAGTTGGTAAACAAATAATCTGCGCATGAGCCAAGGGTATTTACG | 382 | 7119 |
| RP23- | ACTACTTGTACTCTGTTTTTCCCGTGAACGTCAGCTCGGCCATGGGCTGCAGCCAATCAGGGAGTGATGCGTCCGAGGCAATTGTTGTTCTCTTTAAAGAGGA | | |
| 31B18 | AAGGGGTTTCGTTTTCTCTCTCTCTTGCTTCTTACACTCTGGCCCCATAAGATGTAAGCAATAAAGCTTTGCCGTAGAAGATTCTGGGTGTTGTGTTCTTCCTGG | | |
| | CCGGTCGTGAGAACGCGTCGAATAACAATTGGTGCCGAAAACCCGAGACGAGAAAAAACTCCGGACTGGCGCAGGGAAGATCCCTCATTCCGGAACCAGAA | | |
| | CTGCGGATCACGTTTATAAAGGTTCCCGTAACACAGACTGTTGAGAAGGATTCAACTGCCGAATTCAGAACTCATCAGCTGGGGAACGACGGTGATAAAGGT | | |
| | TCCCGTAAAGCAGACTGTTAAGAAGGATTCAACTGTATGAATTCAGAACTTTTCAGCTGGGGAACGAGAGTACCAGTGAGTATGTTTGGCCTTGAATTTTTTC | | |
| | TGGTGTTAGGAGCCCTTTTGTTCTTTTTCACATGTTATCAAGTGATTAAGATAGGGCTGAAAATTCTAGAGGAAATTCAGGACAAGCTATCAGAAGTAAAGCG | | |
| | GGAAGAAAGAGTAGGAACAAAGAGGAAGTATGGTACACAAAATAAGTATACAGGCCTTTCCAAGGGTCTTGAACCCGAGGAAAAGTTAAGGTTAGGTAGG | | |
| | AATACCTGGAGAGAGATTAGAAGAAAAAGAGGAAAAAGGGAAAAGAAGAAAGATCGATTAGCGGAGGTCTCTAGGAGATACTCGTCACTAGATGAGCTCA | | |
| | GGAAGCCAGCTCTTAGTAGCTCTGAAGCAAGTGAAGAATCCTCCTCTGAGGAAACAGACTGGGAGGAAGAAGCAGCCCATTACCAGCCAGCTAATTGGTCA | | |
| | AGAAAAAAGCCAAAAGCGGCTGGCGAAAGTCAGCGTACTGTTCAACCTCCCGGCAGTCGGTTTCAAGGTCCGCCCTATGCGGAGCCCCCGCCCTGCGTAGTG | | |
| | CGTCAGCAATGCGCAGAGAGGCAATGCGCAGAGAGATGCGCAGAGAGGCAGTGCGCAGAGAGATGCGCAGAGAGGCAGTGCGCAGAGAGGCAGTGCGC | | |
| | AGAGAGGCAGTGCGCAGACTCATTCATTCCCCGAGAGGAACAAAAGAAAATACAACAGGCATTTCCAGTCTTTGAAGGAGCCGAGGGTGGGCGTGTCCACG | | |
| | CTCCGGTAGAATACGTACAGATTAAGGAAATTGCCGAGTCGGTTCGTAAATACGGAACCAATGCTAATTTTACCTTGGCGCAGTTAGACAGGCTCGCTGGCA | | |
| | TGGCACTAACGCCTGCTGATTGGCAGACGGTTGTAAAAGCCGCTCTCCCTAGTATGGGCAAATATATGGAATGGAAAGCGCTTTGGCACGAAGCTGCACAGG | | |
| | CGCAGGCCCGAGCAAACGCAGCTGCTTTGACTCCAGAGCAGAGAGATTGGACTTTTGACTTGTTAACGGGTCAGGGAGCTTATTCTGCTGATCAGACAAACT | | |
| | ACCATTGGGGAGCTTATGCCCAGATTTCTTCCACGGCTATTAGGGCCTGGAAGGCGCTCTCTCGAGCAGGTGAAACCACTGGTCAGTTAACAAAGATAATCC | | |
| | AGGGACCTCAGGAATCTTTCTCAGATTTTGTGGCCAGAATGACAGAGGCAGCAGAGCGTATTTTTGGAGAGTCAGAGCAAGCTGCGCCTCTCATAGAACAGC | | |
| | TAATCTACGAGCAAGCCACAAAGGAGTGCCGAGCGGCCATAGCCCCAAGAAAGAACAAAGGCTTACAAGACTGGCTCAGGGTCTGTCGAGAGCTTGGGGG | | |
| | ACCTCTCAGCAATGCAGGTTTAGCGGCTGCCATCCTTCAATCTCAGAACCGCTCCATGGGCAGAAATGATCAGAGGACATGTTTTAATTGCGGAAAGCCTGG | | |
| | GCATTTTAAGAAAGATTGCAGAGCTCCAGATAAACAGGGAGGGACTCTCACTCTTTGCTCTAAGTGTGGCAAGGGTTATCATAGAGCTGACCAGTGTCGCTC | | |
| | TGTGAGGGATATAAAGGGCAGAATTCTTCCCCCACCTGATAGTCAATCAACTGATGTGCCAAAAAACGGGTCATCGGGCCCTCGGTCCCAGGGCCCTCAAAG | | |
| | ATATGGGAACCGGTTTGTCAGGACCCAGGAAGCAGTCAGAGAGACGACCCAGGAAGACCCACAAGGGTGGACCTGCGTGCCGCCTCCGACTTCCTATTAAT | | |
| | GCCTCAAATGAGTATTCAGCCGGTGCCGGTGGAGCCTATACCATCCTTGCCCCCGGGAACCATGGGCCTTATTCTCGGCCGAGGTTCACTCACCTTGCAGGGC | | |
| | TTAGTAGTCCACCCTGGAGTTATGGATTGTCAACATTCCCCTGAAATACAGGTCCTGTGCTCAAGCCCTAAAGGCGTTTTTTCTATTAGTAAAGGAGATAGGAT | | |
| | AGCTCAGCTGCTGCTCCTCCCTGATAATACCAGGGAGAAATCTGCAGGACCTGAGATAAAGAAAATGGGCTCCTCAGGAAATGATTCTGCCTATTTGGTTGTA | | |
| | TCTTTAAATGATAGACCTAAGCTCCGCCTTAAGATTAATGGAAAAGAGTTTGAAGGCATCCTTGATACCGGAGCAGATAAAAGTATAATTTCTACACATTGGT | | |
| | GGCCCAAAGCATGGCCCACCACAGAGTCATCTCATTCATTACAGGGCCTAGGATATCAATCATGTCCCACTATAAGCTCCGTTGCCTTGACGTGGGAATCCTC | | |
| | TGAAGGGCAGCAAGGGAAATTCATACCTTATGTGCTCCCACTCCCGGTTAACCTCTGGGGAAGGGATATTATGCAGCATTTGGGCCTTATTTTGTCCAATGAA | | |
| | AACGCCCCATCAGGAGGGTATTCAGCTAAAGCAAAAAATATCATGGCAAAGATGGGTTATAAAGAAGGAAAAGGGTTAGGACATCAAGAACAGGGAAGGA | | |
| | TAGAGCCTATCTTACCTAATGGAAACCAAGACAGACAGGGTCTGGGTTTTTCCTTAGCGGCCATTGGGGCAGCACGACCCATACCATGGAAAACAGGGGACC | | |
| | CAGTGTGGGTTCCTCAATGGCACCTATCCTCTGAAAAACTAGAAGCTGTGATTCAACTGGTAGAGGAACAATTAAAACTAGGCCATATTGAACCCTCTACCTC | | |
| | ACCTTGGAATACTCCAATTTTTGTAATTAAGAAAAAGTCAGGAAAGTGGAGACTGCTCCATGACCTCAGAGCCATTAATGAGCATATGAACTTATTTGGCCCA | | |
| | GTACAGAGGGGTCTCCCTGTACTTTCCGCCTTACCACGTGGCTGGAATTTAATCATTATAGATATTAAAGATTGTTTCTTTTCTATACCTTTGTGTCCAAGGGAT | | |
| | AGGCCCAGATTTGCCTTTACCATCCCCTCTATTAATCACATGGAACCTGATAAGAGGTATCAATGGAAGGTCTTACCACAGGGAATGTCCAATAGTCCTACTAT | | |
| | GTGTCAACTTTATGTACAAGAAGCTCTTTTGCCAGTGAGGGAACAATTCCCCTCTTTAATTTTGCTCCTTTACATGGATGACATCCTCCTGTGCCATAAAGACCT | | |
| | TACCATGCTACAAAAGGCATATCCTTTTCTACTTAAAACTTTAAGTCAGTGGGGTTTACAGATAGCCACAGAAAAGGTCCAAATTTCTGATACAGGACAATTCT | | |
| | TGGGCTCTGTGGTGTCCCCAGATAAGATTGTGCCCCAAAAGGTAGAGATAAGAAGAGATCACCTCCATACCTTAAATGATTTTCAAAAGCTGTTGGGAGATA | | |
| | TTAATTGGCTCAGACCTTTTTTAAAGATTCCTTCCGCTGAGTTAAGGCCTTTGTTTAGTATTTTAGAAGGAGATCCTCATATCTCCTCCCCTAGGACTCTTACTCT | | |
| | AGCTGCTAACCAGGCCTTACAAAAGGTGGAAAAAGCCTTACAGAATGCACAATTACAACGTATTGAGGATTCGCAGCCTTTCAGTTTGTGTGTCTTTAAGACA | | |
| | GCACAATTACCAACTGCAGTTTTGTGGCAGAATGGGCCATTGTTGTGGATCCATCCAAACGTATCCCCAGCTAAAATAATAGATTGGTATCCTGATGCAATTG | | |
| | CACAGCTTGCCCTTAAAGGCCTAAAAGCAGCAATCACCCACTTTGGGCAAAGTCCATATCTTTTAATTGTACCTTATACCGCTGCACAGGTTCAAACCTTGGCA | | |
| | GCCACATCTAATGATTGGGCAGTTTTAGTTACCTCCTTTTCAGGAAAAATAGATAACCATTATCCAAAACATCCAATCTTACAGTTTGCCCAAAATCAATCTGTT | | |
| | GTGTTTCCACAAATAACAGTAAGAAACCCACTTAAAAATGGGATTGTGGTATATACTGATGGATCAAAAACTGGCATAGGTGCCTATGTGGCTAATGGTAAA | | |
| | GTGGTATCCAAACAGTATAATGAAAATTCACCTCAAGTGGTAGAATGTTTAGTGGTCTTAGAAGTTTTAAAAACCTTTTTAGAACCCCTTAATATTGTGTCAGA | | |
| | TTCCTGTTATGTGGTAAATGCAGTAAATCTTTTAGAAGCGGCTGGAGTGATTAAGCCTTCCAGTAGAGTTGCCAATATTTTTCAGCAGATACAATTAGTTTTGT | | |
| | TATCTAGAAGATCTCCTGTTTATATTACTCATGTTAGAGCCCACTCAGGCCTACCTGGCCCCATGGCTCTGGGAAATGATTTGGCAGATAAGGCCACTAAAGT | | |
| | GGTGGCTGCTGCCCTATCATCCCCGGTAGAGGCTGCAAGAAATTTTCATAACAATTTTCATGTGACGGCTGAAACATTACGCAGTCGTTTCTCCTTGACAAGA | | |
| | AAAGAAGCCCGTGACATTGTTACTCAATGTCAAAGCTGCTGTGAGTTCTTGCCAGTTCCTCATGTGGGAATTAACCCACGCGGTATTCGACCTCTACAGGTCT | | |
| | GGCAAATGGATGTTACACATGTTTCTTCCTTTGGAAAACTTCAATATCTCCATGTGTCCATTGACACATGTTCTGGCATCATGTTTGCTTCTCCGTTAACTGGAG | | |
| | AAAAAGCCTCACATGTGATTCAACATTGTCTTGAGGCATGGAGTGCTTGGGGGAAACCCAGACTCCTTAAGACTGATAATGGACCAGCTTATACGTCTCAAAA | | |
| | ATTTCAACAGTTCTGCCGTCAGATGGACGTAACCCACCTGACTGGACTTCCATACAACCCTCAAGGACAGGGTATTGTTGAGCGTGCGCATCGCACCCTCAAA | | |
| | GCCTATCTTATAAAACAGAAGAGGGGAACTTTTGAGGAGACTGTACCCCGAGCACCAAGAGTGTCGGTGTCTTTGGCACTCTTTACACTCAATTTTTTAAATA | | |
| | TTGATGCTCATGGCCATACTGCGGCTGAACGTCATTGTTCAGAGCCAGATAGGCCCAATGAGATGGTTAAATGGAAAAATGTTCTTGATAATAAATGGTATG | | |
| | GCCCGGATCCTATCTTGATAAGATCCAGGGGAGCTATCTGTGTTTTCCCACAGAATGAAGACAACCCATTTTGGGTACCAGAAAGACTCACCCGAAAAATCCA | | |
| | GACTGACCAAGGGAATACTAATGTCCCTCGTCTTGGTGATGTCCAGGGCGTCAATAATAAAGAGAGAGCAGCGTTGGGGGATAATGTCGACATTTCCACTCC | | |
| | CAATGACGGTGATGTATAATGCTCAAGTATTCTCCTGCTTTTTTACCACTAACTGGGAACTGGGTTTGGCCTTAATTCAGACAGCCTTGGCTCTGTCTGGACAG | | |
| | GTCCAGATGACTGACACCATTAACACTTTGTCAGCCTCAGTGACTACAGTCATAGATGAACAGGCCTCAGCTAATGTCAAGATACAGAGAGGTCTCATGCTGG | | |
| | TTAATCAACTCATAGATCTTGTCCAGAAACAACTAGATGTATTATGACAAATAACTCAGCAGGGATGTGAACAAAAGTTTCCGGGATTGTGTGTTATTTCCATT | | |
| | CAGTATGTTAAATTTACTAGGGCAGCTAATTTGTCAAAAAGTCTTTTTCAGTATATGTTACAGAATTGGATGGCTGAATTTGAACAGATCCTTCGGGAATTGAG | | |
| | ACTTCAGGTCAACTCCACGCGCTTGGACCTGTCGCTGACCAAAGGATTACCCAATTGGATCTCCTCAGCATTTTCTTTCTTTAAAAAATTGGGTGGGATTAATA | | |
| | TTATTTGGAGATACACTTTGCTGTGGATTAGTGTTGCTTCTTTGATTGGTCTGTAAGCTTAAGGCCCAAACTAGGAGAGACAAGGTGGTTATTGCCCAGGCGC | | |
| | TTGCAGGACTAGAACATGGAGCTTCCCCTGATATATCTATGCTTAGGCAATAGGTCGCTGGCCACTCAGCTCTTATATCTCACGAGGCTAGACTCATTGCACG | | |
| | AGATAGAGTGAGTGTGCTTCAGCAGCCCGAGAGAGTTGCAAGGCTAAGCACTGCAGTAGAAGGGCTCTGCGGCACATATGAGCCTATTCTAGGGAGACATG | | |
| | TCATCTTTCATGAAGGTTCAGTGTCCTAGTTCCCTTCCCCCAGGCAAAACGACACGGGAGCAGGTCAGGGTTGCTCTGGGTAAAAGCCTGTGAGCCTAAGAG | | |
| | CTAATCCTGTACATGGTTCCTTTACCTACACACTGGGGATTTGACCTCTATCTCCACTCTCGTTAATATGGGTGGCCTATTTGCTCTTATTAATAGAAAAGGGGG | | |
| | AACTGTGGGAAGCCGCCCCCACATTCGCCGTCACAAGATGGCGCTGACATCCTGTGTTCTAAGTTGGTAAACAAATAATCTGCGCATGAGCCAAGGGTATTTA | | |
| | CGACTACTTGTACTCTGTTTTTCCCGTGAACGTCAGCTCGGCCATGGGCTGCAGCCAATCAGGGAGTGATGCGTCCGAGGCAATTGTTGTTCTCTTTAAAGAG | | |
| | GAAAGGGGTTTCGTTTTCTCTCTCTCTTGCTTCTTACACTCTGGCCCCATAAGATGTAAGCAATAAAGCTTTGCCGTAGAAGATTCTGGGTGTTGTGTTCTTCCT | | |
| | GGCCGGTCGTGAGAACGCGTCGAATAACA | | |
| IAP- | TGTTGGGAGCCGCGCCCACATTCGCCGTTACAAGATGGCGCTGACAGCTGTGTTCTAAGTGGTAAACAAATAATCTGCGCATGTGCCGAGGGTGGTTCTTCA | 383 | 7126 |
| RP23- | CTCCATGTGCTCTGCCTTCCCCGTGACGTCAACTCGGCCGATGGGCTGCAGCCAATCAGGGAGTGACACGTCCTAGGCGAAGGAGAATTCTCTTTAATAGGG | | |
| 324C9 | ACGGGGTTTCGTTTTCTCTCTCTCTTGCTTCTTGCTCTCTTGCTTCTTGCACTCTGGCTCCTGAAGATGTAAGCAATAAAGCTTTGCCGCAGAAGATTCTGGTCT | | |
| | GTGGTGTTCTTCCTGGCCGGTCGTGAGAACGCGTCTAATAACAATTGGTGCCGAATTCCGGGACGAGAAAAAACTCGGGACTGGCGCAAGGAAGATCCCTC | | |
| | ATTCCAGAACCAGAACTGCGGGTCGCGGTAATAAAGGTTCCCGTAAAGCAGACTGTTAAGAAGGATTCAACTGTATGAATTCAGAACTTTTCAGCTGGGGAA | | |
| | CGAGAGTACCAGTGAGTACAGCTTTACGAGGTAAGTCTGATCTTGAACTTTCTAAGGAAATTCAAGACAGTCTATCAGAAGTAAAGTGGAATATGTTTGGCCT | | |
| | TGAATTTTTTCTGGTGTTAGGAGCCCTTTTGTTCCTTTTCACATGTTATCAAGTGATTAAGATAGGGCTGAAAATTCTAGAGGAAATTCAGGACAAGCTATCAG | | |
| | AAGTAAAGCGGGGAGAGAGAGTAGGAGCAAAGAGAAAATATGGTACACAAAATAAGTATACAGGCCTTTCCAAGGGTCTTGAACCCGAGGAAAAGTTAAG | | |
| | GTTAGGTAGGAATACCTGGAGAGAGATTAGAAGAAAAAGAGGAAAAAGGGAAAAGAAGAAAGATCAATTAGCGGAGGTCTCTAGGAAAAGGAGCCTGTG | | |
| | CTCATCGCTGGATGGGCTCGGGGAGCCAGCTCTTAGTAGCTCTGAAGCAGATGAAGAATTCTCCTCTGAAGAAACAGACTGGGAGGAAGAAGCAGCTCATT | | |
| | ATGAGAAAAAAGGGTACCAGCCAGGTAAAGTGCTAGCTAATCAGTTAAGGAAGCCAAAAGCGGCTGGCGAAGGCCAGTTTGCTGATTGGCCTCAGGGCAG | | |
| | TCGGTTTCAAGGTCCGCCCTATGCGGAGCCCCCGCCCTGCGTAGTGCGTCAGCAATGCGCAGAGAGATGCGCAGAGAGGCAGTGCGCAGAGAGGCAGTGC | | |
| | GCAGACTCATTCATTCCCAGAGAGGAACAAAGGAAAATACAACAGGCATTTCCGGTCTTTGAAGGAGCCGAGGGTGGGCGTGTCCACGCTCCGGTAGAATA | | |
| | CTTACAAATTAAAGAAATTGCCGAGTCGGTCCGTAAATACGGAACCAATGCTAATTTTACCTTGGTGCAGTTAGACAGGCTCGCCGGCATGGCACTAACTCCT | | |
| | GCTGACTGGCAAACGGTTGTAAAAGCCGCTCTCCCTAGTATGGGCAAATATATGGAATGGAGAGCGCTTTGGCACGAAGCTGCACAAGCGCAGGCCCGAGC | | |
| | AAACGCAGCTGCTTTGACTCCAGAGCAGAGAGATTGGACTTTTGACTTGTTAACGGGTCAGGGAGCTTATTCTGCTGATCAGACAAACTACCATTGGGGAGC | | |
| | TTATGCCCAGATTTCTTCCACGGCTATTAGGGCCTGGAAGGCGCTCTCTCGAGCAGGTGAAACCACTGGTCAGTTAACAAAGATAATCCAGGGACCTCAGGA | | |
| | ATCCTTCTCAGATTTTGTGGCCAGAATGACAGAGGCAGCAGAGCGTATTTTTGGAGAGTCAGAGCAAGCTGCGCCTCTGATAGAACAGCTAATCTATGAGCA | | |
| | AGCCACAAAGGAGTGCCGAGCGGCCATAGCCCCAAGAAAGAACAAAGGCTTACAAGACTGGCTCAGGGTCTGTCGAGAGCTTGGGGGACCTCTCACCAATG | | |
| | CAGGCTTAGCGGCCGCCATCCTCCAATCTCAGAACCGCTCCATGGGCAGAAATGATCAGAGGACATGTTTTAATTGCGGAAAGCCTGGGCATTTTAAGAAAG | | |
| | ATTGCAGAGCTCCAGATAAACAGGGAGGGACTCTCACTCTTTGCTCTAAGTGTGGCAAGGGTTATCATAGAGCTGACCAGTGTCGCTCTGTGAGGGATATAA | | |
| | AGGGCAGAGTCCTTCCCCCACCTGATAGTCAATCAACTGATGTGCCAAAAAACGGGTCATCGGGCCCTCGGTCCCAGGGCCCTCAAAGATATGGGAACCGGT | | |
| | TTGTCAGGACCCAGGAAGCAGTCAGAGAGGCGACCCAGGAAGACCCACAAGGGTGGACCTGCGTGCCGCCTCCGACTTCCTATTAATGCCTCAAATGAGTAT | | |
| | TCAGCCGGTGCCAGTGGAGCCTATACCATCCTTGCCCCTGGGAACCATGGGCCTTATTCTCGGCCGGGGTTCACTCACCTTGCAGGGCTTAGTAGTCCACCCT | | |
| | GGAGTTATGGATTGTCAACATTCCCCTGAAATACAGGTCCTGTGCTCAAGCCCTAAAGGCGTTTTTTCTATTAGTAAAGGAGATAGGATAGCTCAGCTGCTGC | | |
| | TCCTCCCTGATAATACCAGGGAGAAATCTGCAGGACCTGAGATAAAGAAAATGGGCTCCTCAGGAAATGATTCTGCCTATTTGGTTGTATCTTTAAATGATAG | | |
| | ACCTAAGCTCCGCCTTAAGATCAACGGAAAAGAGTTTGAAGGCATCCTTGATACCGGAGCAGATAAAAGTATAATTTCTACACATTGGTGGCCCAAAGCATG | | |
| | GCCCACCACAGAGTCATCTCATTCATTACAGGGCCTAGGTTATCAATCATGTCCCACTATAAGCTCCATTGCCTTGACGTGGGAATCCTCTGAAGGGCAGCAA | | |
| | GGGAAATTCATACCTTATGTGCTCCCACTCCCGGTTAACCTCTGGGGAAGGGATATTATGCAGCATTTGGGCCTTATTTTGTCCAATGAAAACGCCCCATCGG | | |
| | GAGGGTATTCAACTAAAGCAAAAAATATCATGGCAAAGATGGGTTATAAAGAAGGAAAAGGGTTAGGACATCAAGAACAGGGAAGGATAGAGCCCATCTC | | |
| | ACCTAATGGAAACCAAGACAGACAGGGTCTGGGTTTTCCTTAGCGGCCATTGGGGCAGCACGACCCATACCATGGAAAACAGGGGACCCAGTGTGGGTTCC | | |
| | TCAATGGCACCTATCCTCTGAAAAACTAGAAGCTGTGATTCAACTGGTAGAGGAACAATTAAAATTAGGCCATATTGAACCCTCTACCTCACCTTGGAATACTC | | |
| | CAATTTTTGTAATTAAGAAAAAGTCAGGAAAGTGGAGACTGCTCCATGACCTCAGAGCCATTAATGAGCAAATGAACTTATTTGGCCCAGTACAGAGGGGTC | | |
| | TCCCTGTACTTTCCGCCTTACCACGTGGCTGGAATTTAATTATTATAGATATTAAAGATTGTTTCTTTTCTATACCTTTGTGTCCAAGGGATAGGCCCAGATTTG | | |
| | CCTTTACCATCCCCTCTATTAATCACATGGAACCTGATAAGAGGTATCAATGGAAGGTCTTACCACAGGGAATGTCCAATAGTCCTACAATGTGCCAACTTTAT | | |
| | GTGCAAGAAGCTCTTTTGCCAGTGAGGGAACAATTCCCCTCTTTAATTTTGCTCCTTTACATGGATGACATCCTCCTGTGCCATAAAGACCTTACCATGCTACAA | | |
| | AAGGCATATCCTTTTCTACTTAAAACTTTAAGTCAGTGGGGTTTACAGATAGCCACAGAAAAGGTCCAAATTTCTGATACAGGACAATTCTTGGGCTCTGTGG | | |
| | TGTCCCCAGATAAGATTGTGCCCCAAAAGGTAGAGATAAGAAGAGATCACCTCCATACCTTAAATGATTTTCAAAAGCTGTTGGGAGATATTAATTGGCTCAG | | |
| | ACCTTTTTTAAAGATTCCTTCCGCTGAGTTAAGGCCTTTGTTTAGTATTTTAGAAGGAGATCCTCATATCTCCTCCCCTAGGACTCTTACTCTAGCTGCTAACCAG | | |
| | GCCTTACAAAAGGTGGAAAAGGCCTTACAGAATGCACAATTACAACGTATTGAGGATTCGCAGCCTTTCAGTTTGTGTGTCTTTAAGACAGCACAATTGCCAA | | |
| | CTGCAGTTTTGTGGCAGAATGGGCCATTGTTGTGGATCCATCCAAACGTATCCCCAGCTAAAATAATAGATTGGTATCCTGATGCAATTGCACAGCTTGCCCTT | | |
| | AAAGGCCTAAAAGCAGCAATCACCCACTTTGGGCAAAGTCCATATCCTTTAATTGTACCTTATACCGCTGCACAGGTTCAAACCTTGGCAGCCACATCTAATGA | | |
| | TTGGGCAGTTTTAGTTACCTCCTTTTCAGGAAAAATAGATAACCATTATCCAAAGCATCCAATCTTACAGTTTGCCCAAAATCAATCTGTTGTGTTTCCACAAAT | | |
| | AACAGTAAGAAACCCACTTAAAAATGGGATTGTGGTATATACTGATGGATCAAAAACTGGCATAGGTGCCTATGTGGCTAATGGTAAAGTGGTATCCAAACA | | |
| | ATATAATGAAAATTCACCTCAAGTGGTAGAATGTTTAGTGGTCTTAGAAGTTTTAAAAACCTTTTTAGAACCCCTTAATATTGTGTCAGATTCCTGTTATGTGGT | | |
| | TAATGCAGTAAATCTTTTAGAAGTGGCTGGAGTGATTAAGCCTTCCAGTAGAGTTGCCAATATTTTTCAGCAGATACAATTAGTTTTGTTATCTAGAAGATCTC | | |
| | CTGTTTATATTACTCATGTTAGAGCCCATTCAGGCCTACCTGGCCCCATGGCTCTGGGAAATGATTTGGCAGATAAGGCCACTAAAGTGGTGGCTGCTGCCCT | | |
| | ATCATCCCCGGTAGAGGCTGCAAAAAATTTTCATAACAATTTTCATGTGACGGCTGAAACATTACGCAGTCGTTTCTCCTTGACAAGAAAAGAAGCCCGTGAC | | |
| | ATTGTTACTCAATGTCAAAGCTGCTGTGAGTTCTTGCCAGTTCCTCATGTGGGAATTAACCCACGCGGTATTCGACCTCTACAGGTCTGGCAAATGGATGTTAC | | |
| | ACATGTTTCTTCCTTTGGAAAACTTCAATATCTCCATGTGTCCATTGACACATGTTCTGGCATCATGTTTGCTTCTCCGTTAACCGGAGAAAAAGCCTCACATGT | | |
| | GATTCAACATTGCCTTGAGGCATGGAGTGCTTGGGGGAAACCCAGACTCCTTAAGACTGATAATGGACCAGCTTATACGTCTCAAAAATTCCAACAGTTCTGC | | |
| | CGTCAGATGGACGTGACCCACCTGACTGGACTTCCATACAACCCTCAAGGACAGGGTATTGTTGAGCGTGCGCATCGCACCCTCAAAACCTATCTTATAAAAC | | |
| | AGAAGAGGGGAACTTTTGAGGAGACTGTACCCCGAGCACCAAGAGTGTCTGTGTCTTTGGCACTCTTTACACTCAATTTTTTAAATATTGATGCTCATGGCCA | | |
| | TACTGCGGCTGAACGTCATTGTACAGAGCCAGATAGGCCCAATGAGATGGTTAAATGGAAAAATGTCCTTGATAATAAATGGTATGGCCCGGATCCTATTTT | | |
| | GATAAGATCCAGGGGAGCTATCTGTGTTTTCCCACAGAATGAAGACAACCCATTTTGGGTACCAGAAAGACTCACCCGAAAAATCCAGACTGACCAAGGGAA | | |
| | TACTAATGTCCCTCGTCTTGGTGATGTCCAGGGCGTTAATAATAAAGAGAGAGCAGCGTTGGGGGATAATGTCGACATTTCCACTCCCAATGACGGTGATGT | | |
| | ATAATGCTCAAGTATTCTCCTGCTTTTTTACCACTAACTGGGAACTGGGTTTGGCCTTGATTCAGACAGCCTTGGCTCTGTCTGGACAGGTCCAGACGACTGAC | | |
| | ACCATTAACACTTTGTCAGCCTCAGTGACTACAGTCATAGATAAACAGGCCTCAGCTAATGTCAAGATACAGAGAGGTCTCATGCTGGTTAATCAACTCATAG | | |
| | ATCTTGTCCAGATACAACTAGATGTATTATGACAAATAACTCAGCTGGGATGTGAACAAAAGTTTCCGGAATTGTGTGTTATTTCCATTCAGTATGTTAAATTT | | |
| | ACTAGGGCAGCTAATTTGTCAAAAAGTCTTTTTCAGTATATGTTACAGAATTGGATGGCTGAATTTGAACAGATCCTTCGGGAATTGAGACTTCAGGTCAACT | | |
| | CCACGCGCTTGGACCTGTCGCTGACCAAAGGATTACCCAATTGGATCTCCTCAGCATTTTCCTTCTTTAAAAAATGGGTGGGATTAATATTATTTGGAGATACA | | |
| | CTTTGCTGTGGATTAGTGTTGCTTCTTTGATTGGTCTGTAAGCTTAAGGCCCAAACTAGGAGAGACAAGGTGGTTATTGCCCAGGCGCTTGCAGGACTAGAAC | | |
| | ATGGAGCTCCCCCTGATATATGGTTATCTATGCTTAGGCAATAGGTCGCTGGCCACTCAGCTCTTATATCCCACGAGGCTAGTCTCATTGCACGGGATAGAGT | | |
| | GAGTGTGCTTCAGCAGCCCGAGAGAGTTGCACGGCTAAGCACTGCAATGGAAAGGCTCTGCGGCATATATGAGCCTATTCTAGGGAGACATGTCATCTTTCA | | |
| | TGAAGGTTCAGTGTCCTAGTTCCCTTCCCCCAGGCAAAACGACACGGGAGCAGGTCAGGGTTGCTCTGGGTAAAAGCCTGTGAGCCTAAGAGCTAATCCTGT | | |
| | ACATGGCTCCTTTACCTACACACTGGGGATTTGACCTCTATCTCCACTCTCATTAATATGGGTGGCCTATTTGCTCTTATTAAAAGAAAAGGGGGAGATGTTGG | | |
| | GAGCCGCGCCCACATTCGCCGTTACAAGATGGCGCTGACAGCTGTGTTCTAAGTGGTAAACAAATAATCTGCGCATGTGCCGAGGGTGGTTCTTCACTCCAT | | |
| | GTGCTCTGCCTTCCCCGTGACGTCAACTCGGCCGATGGGCTGCAGCCAATCAGGGAGTGACACGTCCTAGGCGAAGGAGAATTCTCTTTAATAGGGACGGG | | |
| | GTTTCGTTTTCTCTCTCTCTTGCTTCTCGCTCGCTCTTGCTTCTTGCACTCTGGCTCCTGAAGATGTAAGCAATAAAGTTTTGCCGCAGAAGATTCTGGTCTGTG | | |
| | GTGTTCTTCCTGGCCGGTCGTGAGAACGCGTCTAATAACA | | |
| IAP- | TGTGGGAAGCCGCCCCCACATTCGCCGTCACAAGATGGCGCTGACATCCTGTGTTCTAAGTTGGTAAACAAATAATCTGCGCATGAGCCAAGGGTATTTACG | 384 | 7155 |
| RP23- | ACTACTTGTACTCTGTTTTTCCCGTGAACGTCAGCTCGGCCATGGGCTGCAGCCAATCAGGGAGTGATGCGTCCTAGGCAATTGTTGTTCTCTTTAAAGAGGG | | |
| 440N1 | AAGGGGGTTTCGTTTTCTCTCTCTCTTGCTTCGCTCTCTCTTGCTTCTTACACTCTGGCCCCATAAGATGTAAGCAATAAAGCTTTGCCGTAGAAGATTCTGGTT | | |
| | GTTGTGTTCTTCCTGGCCGGTCGTGAGAACGCGTCGAATAACAATTGGTGCCGAAACCCGGGACGAGAAAAAACTCCGGACTGGCGCAGGAGGGATACTTC | | |
| | ATTTCAGAACCAGAACTGCGGATCACGTTTATAAAGGTTCCCGTAACACAGACTGTTGAGAAGGATTCAACTGCCGAATTCAGAACTCATCAGCTGGGGAAC | | |
| | GACGGTGATAAAGGTTCCCGTAAAGCAGACTGTTAAGAAGGATTCAACTGTATGAATTCAGAACTTTTCAGCTGGGGAACGAGAGTACCAGTGAGTATGTTT | | |
| | GGCCTTGAATTTTTTCTGGTGTTAGGAGCCCTTTTGTTCTTTTTCACATGTTATCAAGTGATTAAGATAGGGCTGAAAATTCTAGAGGAAATTCAGGACAAGCT | | |
| | ATCAGAAGTAAAGCGGGAAGAAAGAGTAGGAACAAAGAGGAAGTATGGTACACAAAATAAGTATACAGGCCTTTCCAAGGGTCTTGAACCCGAGGAAAAG | | |
| | TTAAGGTTAGGTAGGAATACCTGGAGAGAGATTAGAAGAAAAAGAGGAAAAAGGGAGAAGAAAAAAGATCGATTAGCGGAGGTCTCTAGGAGATACTCGT | | |
| | CACTAGATGAGCTCAGGAAGCCAGCTCTTAGTAGCTCTGAAGCAAGTGAAGAATCCTCCTCTGAGGAAACAGACTGGGAGGAAGAAGCAGCCCATTACCAG | | |
| | CCAGCTAATTGGTCAAGAAAAAAGCCAAAAGCGGCTGGCGAAAGTCAGCGTACTGTTCAACCTCCCGGCAGTCGGTTTCAAGGTCCGCCCTATGCGGAGCCC | | |
| | CCGCCCTGCGTAGTGCGTCAGCAATGCGCAGAGAGGCAGTGCGCAGAGAGATGCGCAGAGAGGCAGTGCGCAGAGAGATGCGCAGAGAGGCAGTGCGCA | | |
| | GAGAGGCAGTGCGCAGAGAGGCAGTGCGCAGACTCATTCATTCCCCGAGAGGAACAAAAGAAAATACAACAGGCATTTCCAGTCTTTGAAGGAGCCGAGG | | |
| | GTGGGCGTGTCCACGCTCCGGTAGAATACGTACAGATTAAGGAAATTGCCGAGTCGGTTCGTAAATACGGAACCAATGCTAATTTTACCTTGGCGCAGTTAG | | |
| | ACAGGCTCGCTGGCATGGCACTAACTCCTGCCGACTGGCAAACGGTTGTAAAAGCCGCTCTCCCTAGTATGGGCAAATATATGGAATGGAAAGCGCTTTGGC | | |
| | ACGAAGCTGCACAGGCGCAGGCCCGAGCAAACGCAGCTGCTTTGACTCCAGAGCAGAGAGATTGGACTTTTGACTTGTTAACGGGTCAGGGAGCTTATTCT | | |
| | GCTGATCAGACAAACTACCATTGGGGAGCTTATGCCCAGATTTCTTCCACGGCTATTAGGGCCTGGAAGGCGCTCTCTCGAGCAGGTGAAACCACTGGTCAG | | |
| | TTAACAAAGATAATCCAGGGACCTCAGGAATCTTTCTCAGATTTTGTGGCCAGAATGACAGAGGCAGCAGAGCGTATTTTTGGAGAGTCAGAGCAAGCTGCG | | |
| | CCTCTCATAGAACAGCTAATCTACGAGCAAGCCACAAAGGAGTGCCGAGCGGCCATAGCCCCAAGAAAGAACAAAGGCTTACAAGACTGGCTCAGGGTCTG | | |
| | TCGAGAGCTTGGGGGACCTCTCAGCAATGCAGGTTTAGCGGCTGCCATCCTTCAATCTCAGAACCGCTCCATGAGCAGAAATAATCAGAGGACATGTTTTAAT | | |
| | TGCGGAAAGCCTGGGCATTTTAAGAAAGATTGCAGAGCTCCAGATAAACAGGGAGGGACTCTCACTCTTTGCTCTAAGTGTGGCAAGGGTTATCATAGAGCT | | |
| | GACCAGTGTCGCTCTGTGAGGGATATAAAGGGCAGAATTCTTCCCCCACCTGATAGTCAATCAACTGATGTGCCAAAAAACGGGTCATCGGGCCCTCGGTCC | | |
| | CAGGGCCCTCAAAGATATGGGAACCGGTTTGTCAGGACCCAGGAAGCAGTCAGAGAGACGACCCAGGAAGACCCACAAGGGTGGACCTGCGTGCCGCCTC | | |
| | CGACTTCCTATTAATGCCTCAAATGAGTATTCAGCCGGTGCCGGTGGAGCCTATACCATCCTTGCCCCCGGGAACCATGGGCCTTATTCTCGGCCGAGGTTCA | | |
| | CTCACCTTGCAGGGCTTAGTAGTCCACCCTGGAGTTATGGATTGTCAACATTCCCCTGAAATACAGGTCCTGTGCTCAAGCCCTAAAGGCGTTTTTTCTATTAG | | |
| | TAAAGGAGATAGGATAGCTCAGCTGCTGCTCCTCCCTGATAATACCAGGGAGAAATCTGCAGGACCTGAGATAAAGAAAATGGGCTCCTCAGGAAATGATTC | | |
| | TGCCTATTTGGTTGTATCTTTAAATGATAGACCTAAGCTCCGCCTTAAGATTAATGGAAAAGAGTTTGAAGGCATCCTTGATACCGGAGCAGATAAAAGTATA | | |
| | ATTTCTACACATTGGTGGCCCAAAGCATGGCCCACCACAGAGTCATCTCATTCATTACAGGGCCTAGGATATCAATCATGTCCCACTATAAGCTCCGTTGCCTT | | |
| | GACGTGGGAATCCTCTGAAGGGCAGCAAGGGAAATTCATACCTTATGTGCTCCCACTCCCGGTTAACCTCTGGGGAAGGGATATTATGCAGCATTTGGGCCT | | |
| | TATTTTGTCCAATGAAAACGCCCCATCAGGAGGGTATTCAGCTAAAGCAAAAAATATCATGGCGAAGATGGGTTATAAAGAAGGAAAAGGGTTAGGACATC | | |
| | AAGAACAGGGAAGGATAGAGCCCATCTCACCTAATGGAAACCAAGACAGACAGGGTCTGGGTTTTCCTTAGCGGCCATTGGGGCAGCACGACCCATACCAT | | |
| | GGAAAACAGGGGACCCAGTGTGGGTTCCTCAATGGCACCTATCCTCTGAAAAACTAGAAGCTGTGATTCAACTGGTAGAGGAACAATTAAAACTAGGCCATA | | |
| | TTGAACCCTCTACCTCACCTTGGAATACTCCAATTTTTGTAATTAAGAAAAAGTCAGGAAAGTGGAGACTGCTCCATGACCTCAGAGCCATTAATGAGCAAAT | | |
| | GAACTTATTTGGCCCAGTACAGAGGGGTCTCCCTGTACTTTCCGCCTTACCACGTGGCTGGAATTTAATTATTATAGATATTAAAGATTGTTTCTTTTCTATACC | | |
| | TTTGTGTCCAAGGGATAGGCCCAGATTTGCCTTTACCATCCCCTCTATTAATCACATGGAACCTGATAAGAGGTATCAATGGAAGGTCTTACCACAGGGAATG | | |
| | TCCAATAGTCCTACTATGTGTCAACTTTATGTACAAGAAGCTCTTTTGCCAGTGAGGGAACAATTCCCCTCTTTAATTTTGCTCCTTTACATGGATGACATCCTC | | |
| | CTGTGCCATAAAGACCTTACCATGCTACAAAAGGCATATCCTTTTCTACTTAAAACTTTAAGTCAGTGGGGTCTACAGATAGCCACAGAAAAGGTCCAAATTTC | | |
| | TGATACAGGACAATTCTTGGGCTCTGTGGTGTCCCCAGATAAGATTGTGCCCCAAAAGGTAGAGATAAGAAGAGATCACCTCCATACCTTAAATGATTTTCAA | | |
| | AAGCTGTTGGGAGATATTAATTGGCTCAGACCTTTTTTAAAGATTCCTTCCGCTGAGTTAAGGCCTTTGTTTAGTATTTTAGAAGGAGATCCTCATATCTCCTCC | | |
| | CCTAGGACTCTTACTCTAGCTGCTAACCAGGCCTTACAAAAAGTGGAAAAGGCCTTACAGAATGCACAATTACAACGTATTGAGGATTCGCAGCCTTTCAGTT | | |
| | TGTGTGTCTTTAAGACAGCACAATTGCCAACTGCAGTTTTGTGGCAGAATGGGCCATTGTTGTGGATCCATCCAAACGTATCCCCAGCTAAAATAATAGATTG | | |
| | GTATCCTGATGCAATTGCACAGCTTGCCCTTAAAGGCCTAAAAGCAGCAATCACTCACTTTGGGCAAAGTCCATATCTTTTAATTGTACCTTATACCGCTGCAC | | |
| | AGGTTCAAACCTTGGCAGCCGCATCTAATGATTGGGCAGTTTTAGTTACCTCCTTTTCAGGAAAAATAGATAACCATTATCCAAAACATCCAATCTTACAGTTT | | |
| | GCCCAAAATCAATCTGTTGTGTTTCCACAAATAACAGTAAGAAACCCACTTAAAAATGGGATTGTGGTATATACTGATGGATCAAAAACTGGCATAGGTGCCT | | |
| | ATGTGGCTAATGGTAAAGTGGTATCCAAACAATATAATGAAAATTCACCTCAAGTGGTAGAATGTTTAGTGGTCTTAGAAGTTTTAAAAACCTTTTTAGAACC | | |
| | CCTTAATATTGTGTCAGATTCCTGTTATGTGGTAAATGCAGTAAATCTTTTAGAAGTGGCTGGAGTGATTAAGCCTTCCAGTAGAGTTGCCAATATTTTTCAGC | | |
| | AGATACAATTAGTTTTGTTATCTAGAAGATCTCCTGTTTATATTACTCATGTTAGAGCCCATTCAGGCCTACCTGGCCCCATGGCTCTGGGAAATGATTTGGCA | | |
| | GATAAGGCCACTAAAGTGGTGGCTGCTGCCCTATCATCCCCGGTAGAGGCTGCAAGAAATTTTCATAATAATTTTCATGTGACGGCTGAAACATTACGCAGTC | | |
| | GTTTCTCCTTGACAAGAAAAGAAGCCCGTGACATTGTTACTCAATGTCAAAGCTGCTGTGAGTTCTTGCCAGTTCCTCATGTGGGAATTAACCCACGCGGTATT | | |
| | CGACCTCTACAGGTCTGGCAAATGGATGTTACACATGTTTCTTCCTTTGGAAAACTTCAATATCTCCATGTGTCCATTGACACATGTTCTGGCATCATGTTTGCC | | |
| | TCTCCGTTAACCGGAGAAAAAGCCTCACATGTGATTCAACATTGTCTTGAGGCATGGAGTGCTTGGGGGAAACCCAGACTCCTTAAGACTGATAATGGACCA | | |
| | GCTTATACGTCCCAAAAATTTCAGCAGTTCTGCCGTCAGATGGACGTAACCCACCTGACTGGACTTCCATACAACCCTCAAGGACAGGGTATTGTTGAGCGTG | | |
| | CGCATCGCACCCTCAAAGCCTATCTTATAAAACAGAAGAGGGGAACTTTTGAGGAGACTGTACCCCGAGCACCAAGAGTGTCGGTGTCTTTGGCACTCTTTAC | | |
| | ACTCAATTTTTTAAATATTGATGCTCATGGCCATACTGCGGCTGAACGTCATTGTTCAGAGCCAGATAGGCCCAATGAGATGGTTAAATGGAAAAATGTTCTT | | |
| | GATAATAAATGGTATGGCCCGGATCCTATCTTGATAAGATCCAGGGGAGCTATCTGTGTTTTCCCACAGAATGAAGACAACCCATTTTGGGTACCAGAAAGAC | | |
| | TCACCCGAAAAATCCAGACTGACCAAGGGAATACTAATGTCCCTCGTCTTGGTGATGTCCAGGGCGTCAATAATAAAGAGAGAGCAGCGTTGGGGGATAAT | | |
| | GTCGACATTTCCACTCCCAATGACGGTGATGTATAATGCTCAAGTATTCTCTTGCTTTTTTACCACTAACTGGGAACTGGGTTTGGCCTTAATTCAGACAGCCTT | | |
| | GGTTCTGTCTGGACAGGTCCAGATGACTGACACCATTAACACTTTGTCAGCCTCAGTGACTACAGTCATAGATGAACAGGCCTCAGCTAATGTCAAGATACAG | | |
| | AGAGGTCTCATGCTGGTTAATCAACTCATAGATCTTGTCCAGAAACAACTGGATGTATTATGACAAATAACTCAGCAGGGATGTGAACAAAAGTTTCCGGGAT | | |
| | TGTGTGTTATTTCCATTCAGTATGTTAAATTTACTAGGACAGCTAATTTGTCAAAAAGTCTTTTTCAGTATATGTTACAGAATTGGATGGCTGAATTTGAACAGA | | |
| | TCCTTCGAGAATTGAGACTTCAGGTCAACTCCACGCGCTTGGACCTGTCTCTGACCAAAGGATTACCCAATTGGATCTCCTCAGCATTTTCTTTCTTTAAAAAAT | | |
| | TGGGTGGGATTAATATTATTTGGAGATACACTTTGCTGTGGATTAGTGTTGCTTCTTTGATTGGTCTGTAAGCTTAAGGCCCAAACTAGGAGAGACAAGGTGG | | |
| | TTATTGCCCAGGCGCTTGCAGGACTAGAACATGGAGCTTCCCCTGATATATGGTTATCTATGCTTAGGCAATAGGTCGCTGGCCACTCAGCTCTTATATCTCAC | | |
| | GAGGCTAGACTCATTGCACGAGATAGAGTGAGTGTGCTTCAGCAGCCCGAGAGAGTTGCAAGGCTAAGCACTGCAGTAGAAGGGCTCTGCGGCACATATGA | | |
| | GCCTATTCTAGGGAGACATGTCATCTTTCAAGAAGGTTCAGTGTCCTAGTTCCCTTCCCCCAGGCAAAACGACACGGGAGCAGGTCAGGGTTGCTCTGGGTA | | |
| | AAAGCCTGTGAGCCTAAGAGCTAATCCTGTACATGGCTCCTTTACCTACACACTGGGGATTTGACCTCTATCTCCACTCTCATTAATATGGGTGGCCTATTTGCT | | |
| | CTTATTAATAGAAAAGGGGGAACTGTGGGAAGCCGCCCCCACATTCGCCGTCACAAGATGGCGCTGACATCCTGTGTTCTAAGTTGGTAAACAAATAATCTG | | |
| | CGCATGAGCCAAGGGTATTTACGACTACTTGTACTCTGTTTTTCCCGTGAACGTCAGCTCGGCCATGGGCTGCAGCCAATCAGGGAGTGATGCGTCCTAGGCA | | |
| | ATTGTTGTTCTCTTTAAAGAGGGAAGGGGGTTTCGTTTTCTCTCTCTCTTGCTTCGCTCTCTCTTGCTTCTTACACTCTGGCCCCATAAGATGTAAGCAATAAAG | | |
| | CTTTGCCGTAGAAGATTCTGGTTGTTGTGTTCTTCCTGGCCGGTCGTGAGAACGCGTCGAATAACA | | |
| IAP- | TGTTGGGAGCCGCGCCCACATTCGCCGTTACAAGATGGCGCTGACAGCTGTGTTCTAAGTGGTAAACAAATAATCTGCGCATGTGCCGAGGGTGGTTCTTCA | 385 | 7084 |
| RP23- | CTCTATGTGCTCTGCCTTCCCCGTGACGTCAACTCGGCCGATGGGCTGCAGCCAATCAGGGAGTGACACGTCCTAGGCGAAGGAGAATTCTCTTTAATAGGG | | |
| 92L23 | ACGGGGTTTCGTTTTCTCTCTCTCTTGCTTCTCGCTCGCTCTTGCTTCTTGCACTCTGGCTCCTGAAGATGTAAGCAATAAAGTTTTGCCGCAGAAGATTCTGGT | | |
| | CTGTGGTGTTCTTCCTGGCCGGGCGTGAGAACGCGTCTAATAACAATTGGTGCCGAATTCCGGGACGAGAAAAAAACTCGGGACTGGCGCAAGGAAGATCC | | |
| | CTCATTCCAGAACCAGAACTGCGGGTCGCGGTAATAAAGGTTCCCGTAAAGCAGACTGTTAAGAAGGATTCAACTGTATGAATTCAGAACTTTTCAGCTGGG | | |
| | GAACGAGAGTACCAGTGAGTACAGCTTTACGAGGTAAGTCTGATCTTGAACTTTCTAAGGAAATTCAAGACAGTCTATCAGAAGTAAAGTGGAATATGTTTG | | |
| | GCCTTGAATTTTTTCTGGTGTTAGGAGCCCTTTTGTTCCTTTTCACATGTTATCAAGTGATTAAGATAGGGCTGAAAATTCTAGAGGAAATTCAGGACAAGCTA | | |
| | TCAGAAGTAAAGCGGGGAGAGAGAGTAGGAGCAAAGAGAAAATATGGTACACAAAATAAGTATACAGGCCTTTCCAAGGGTCTTGAACCCGAGGAAAAGT | | |
| | TAAGGTTAGGTAGGAATACCTGGAGAGAGATTAGAAGAAAAAGAGGAAAAAGGGAAAAGAAGAAAGATCAATTAGCGGAGGTCTCTAGGAGATACTCGTC | | |
| | ACTAGATGAGCTCAGGAAGCCAGCTCTTAGTAGTTCTGAAGCAGATGAAGAATTCTCCTCTGAGGAAACAGACTGGGAGGAAGAAGCAGCCCATTACCAGC | | |
| | CAGCTAATTGGTCAAGAAAAAAGCCAAAAGCGGCTGGCGAAGGCCAGTTTGCTGATTGGCCTCAGGGCAGTCGGCTTCAAGGTCCGCCCTATGCGGAGTCC | | |
| | CCGCCCTGCGTAGTGCGTCAGCAATGCGCAGAGAGGCAGTGCGCAGAGAGGCAGTGCGCAGACTCATTCATTCCCAGAGAGGAACAAAGGAAAATACAAC | | |
| | AGGCATTTCCGGTCTTTGAAGGAGCCGAGGGTGGGCGTGTCCACGCTCCGGTAGAATACTTACAAATTAAAGAAATTGCCGAGTCGGTCCGTAAATATGGAA | | |
| | CCAATGCTAATTTTACCTTGGTGCAGTTAGACAGGCTCGCCGGCATGGCACTAACTCCTGCTGACTGGCAAACGGTTGTAAAAGCCGCTCTCCCTAGTATGGG | | |
| | CAAATATATGGAATGGAGAGCGCTTTGGCACGAAGCTGCACAAGCGCAGGCCCGAGCAAACGCAGCTGCTTTGACTCCAGAGCAGAGAGATTGGACTTTTG | | |
| | ACTTGTTAACGGGTCAGGGAGCTTATTCTGCTGATCAGACAAACTACCATTGGGGAGCTTATGCCCAGATTTCTTCCACGGCTATTAGGGCCTGGAAGGCGCT | | |
| | CTCTCGAGCAGGTGAAACCACTGGTCAGTTAACAAAGATAATCCAGGGACCTCAGGAATCCTTCTCAGATTTTGTGGCCAGAATGACAGAGGCAGCAGAGCG | | |
| | TATTTTTGGAGAGTCAGAGCAAGCTGCGCCTCTGATAGAACAGCTAATCTATGAGCAAGCCACAAAGGAGTGCCGAGCGGCCATAGCCCCAAGAAAGAACA | | |
| | AAGGCTTACAAGACTGGCTCAGGGTCTGTCGAGAGCTTGGGGGACCTCTCAGCAATGCAGGTTTAGCGGCTGCCATCCTTCAATCCCAAAACCGCTCCATGG | | |
| | GCAGAAATAATCAGAGGACATGTTTTAACTGCGGAAAGCCTGGGCATTTTAAGAAAGATTGCAGAGCTCCAGATAAACAGGGAGGGACTCTCACTCTTTGCT | | |
| | CTAAGTGTGGCAAGGGTTATCATAGAGCTGACCAGTGTCGCTCTGTGAGGGATATAAAGGGCAGAGTCCTTCCCCCACCTGATAGTCAATCAGCTGATGTGC | | |
| | CAAAAAACGGGTCATCGGGCCCTCGGTCCCAGGGCCCTCAAAGATATGGGAACCGGTTTGTCAGGACCCAGGAAGCAGTCAGAGAGGCGACCCAGGAAGA | | |
| | CCCACAAGGGTGGACCTGCGTGCCGCCTCCGACTTCCTACTAATGCCTCAAATGAGTATTCAGCCGGTGCCGGTGGAGCCTATACCATCCTTGCCCCCGGGAA | | |
| | CCATGGGCCTTATTCTCGGCCGGGGTTCACTCACCTTGCAGGGCTTAGTAGTCCACCCTGGAGTTATGGATTGTCAACATTCCCCTGAAATACAGGTCCTGTGC | | |
| | TCAAGCCCTAAAGGCGTTTTTTCTATTAGTAAAGGAGATAGGATAGCTCAGCTGCTGCTCCTCCCTGATAATACCAGGGAGAAATTTGCAGGACCTGAGATAA | | |
| | AGAAAATGGGCTCCTCAGGAAATGATTCTGCCTATTTGGTTGTATCTTTAAATGATAGACCTAAGCTCCGCCTTAAGATTAACGGAAAAGAGTTTGAAGGCAT | | |
| | CCTTGATACCGGAGCAGATAAAAGTATAATTTCTACACATTGGTGGCCCAAAGCATGGCCCACCACAGAGTCATCTCATTCATTACAGGGCCTAGGATATCAA | | |
| | TCATGTCCCACTATAAGCTCCATTGCCTTGACGTGGGAATCCTCTGAAGGACAGCAAGGGAAATTCATACCTTATGTGCTCCCACTCCCGGTTAACCTCTGGG | | |
| | GAAGGGATATTATGCAGCATTTGGGCCTTATTTTGTCCAATGAAAACGCCCCATCAGGAGGGTATTCAGCTAAAGCAAAAAATATCATGGCAAAGATGGGTT | | |
| | ATAAAGAAGGAAAAGGGTTAGGACATCAAGAACAGGGAAGGATAGAGCCCATCTCACCTAATGGAAACCAAGACAGACAGGGTCTGGGTTTTCCTTAGCG | | |
| | GCCATTGGGGCAGCACGACCCATACCATGGAAAACAGGGGACCCAGTGTGGGTTCCTCAATGGCACCTATCCTCTGAAAAACTAGAAGCTGTGATTCAACTG | | |
| | GTAGAGGAACAATTAAAACTAGGCCATATTGAACCCTCTACCTCACCTTGGAATACTCCAATTTTTGTAATTAAGAAAAAGTCAGGAAAGTGGAGACTGCTCC | | |
| | ATGACCTCAGAGCCATTAATGAGCAAATGAACTTATTTGGCCCAGTACAGAGGGGTCTCCCTGTACTTTCCGCCTTACCACGTGGCTGGAATTTAATTATTATA | | |
| | GATATTAAAGATTGTTTCTTTTCTATACCTTTGTGTCCAAGGGATAGGCCCAGATTTGCCTTTACCATCCCCTCTATTAATCACATGGAACCTGATAAGAGGTAT | | |
| | CAATGGAAGGTCTTACCACAGGGAATGTCCAATAGTCCTACAATGTGCCAACTTTATGTGCAAGAAGCTCTTTTGCCAGTGAGGGAACAATTCCCCTCTTTAA | | |
| | TTTTGCTCCTTTACATGGATGACATCCTCCTGTGCCATAAAGACCTTACCATGCTACAAAAGGCATATCCTTTTCTACTTAAAACTTTAAGTCAGTGGGGTTTAC | | |
| | AGATAGCCACAGAAAAGGTCCAAATTTCTGATACAGGACAATTCTTGGGCTCTGTGGTGTCCCCAGATAAGATTGTGCCCCAAAAGGTAGAGATAAGAAGAG | | |
| | ATCACCTCCATACCTTAAATGATTTTCAAAAGCTGTTGGGAGATATTAATTGGCTCAGACCTTTTTTAAAGATTCCTTCCGCTGAGTTAAGGCCTTTGTTTAGTA | | |
| | TTTTAGAAGGAGATCCTCATATCTCCTCCCCTAGGACTCTTACTCTAGCTGCTAACCAGGCCTTACAAAAGGTGGAAAAAGCCTTACAGAATGCACAATTACAA | | |
| | CGTATTGAGGATTCGCAGCCTTTCAGTTTGTGTGTCTTTAAGACAGCACAATTGCCAACTGCAGTTTTGTGGCAGAATGGGCCATTGTTGTGGATCCATCCAA | | |
| | ACGTATCCCCAGCTAAAATAATAGATTGGTATCCTGATGCAATTGCACAGCTTGCCCTTAAAGGTCTAAAAGCAGCAATCACCCACTTTGGGCAAAGTCCATA | | |
| | TCTTTTAATTGTACCTTATACCGCTGCACAGGTTCAAACCTTGGCAGCCACATCTAATGATTGGGCAGTTTTAGTTACCTCCTTTTCAGGAAAAATAGATAACCA | | |
| | TTATCCAAAACATCCAATCTTACAGTTTGCCCAAAATCAATCTGTTGTGTTTCCACAAATAACAGTAAGAAACCCACTTAAAAATGGGATTGTGGTATATACTG | | |
| | ATGGATCAAAAACTGGCATAGGTGCCTATGTGGCTAATGGTAAAGTGGTATCCAAACAATATAATGAAAATTCACCTCAAGTGGTAGAATGTTTAGTGGTCTT | | |
| | AGAAGTTTTAAAAACCTTTTTAGAACCCCTTAATATTGTGTCAGATTCCTGTTATGTGGTTAATGCAGTAAATCTTTTAGAAGTGGCTGGAGTGATTAAGCCTT | | |
| | CCAGTAGAGTTGCCAATATTTTTCAGCAGATACAATTAGTTTTGTTATCTAGAAGATTTCCTGTTTATATTACTCATGTTAGAGCCCATTCAGGCCTACCTGGCC | | |
| | CCATGGCTCTGGGAAATAATTTGGCAGATAAGGCCACTAAAGTGGTGGCTGCTGCCCTATCATCCCCGGTAGAGGCTGCAAGAAATTTTCATAACAATTTTCA | | |
| | TGTGACGGCTGAAACATTACGCAGTCGTTTCTCCTTGACAAGAAAAGAAGCCCGTGACATTGTTACTCAATGTCAAAGCTGCTGTGAGTTCTTGCCAGTTCCTC | | |
| | ATGTGGGAATTAACCCACGCGGTATTCGACCTCTACAGGTCTGGCAAATGGATGTTACACATGTTTCTTCCTTTGGAAAACTTCAATATCTCCATGTGTCCATT | | |
| | GACACATGTTCTGGCATCATGTTTGCTTCTCCATTAACCGGAGAAAAAGCCTCACATGTGATTCAACATTGCCTTGAGGCATGGAGTGCTTGGGGGAAACCCA | | |
| | GACTCCTTAAGACTGATAATGGACCAGCTTATACGTCTCAAAAATTCCAACAGTTCTGCCGTCAGATGGACGTGACCCACCTGACTGGACTTCCATACAACCCT | | |
| | CAAGGACAGGGTATTGTTGAGCGTGCGCATCGCACCCTCAAAACCTATCTTATAAAACAGAAGAGGGGAACTTTTGAGGAGACTGTACCCCGAGCACCAAG | | |
| | AGTGTCGGTGTCTATGGCACTCTTTACACTCAATTTTTTAAATATTGATGCTCATGGCCATACTGCGGCTGAACGTCATTGTACAGAGCCAGATAGGCCCAATG | | |
| | AGATGGTTAAATGGAAAAATGTCCTTGATAATAAATGGTATGGCCCGGATCCTATTTTGATAAGATCCAGGGGAGCTATCTGTGTTTTCCCACAGAATGAAAA | | |
| | CAACCCATTTTGGATACCAGAAAGACTCACCCGAAAAATCCAGACTGACCAAGGAAATACTAATGTCCCTCGTCTTGGTGATGTCCAGGGCGTCAATAATAAA | | |
| | AAGAGAGCAGCGTTGGGGGATAATGTCGACATTTCCACTCCCAATGACGGTGATGTATAATGCTCAAGTATTCTCCTGCTTTTTTACCACTAACTAGGAACTG | | |
| | GGTTTGGCCTTAATTCAGACAGCCTTGGCTCTGTCTGGACAGGTCCAGACAACTGACACCATTAACACTTTGTCAGCCTCAGTGACTACAGTCATAGATGAAC | | |
| | AGGCCTCAGCTAATGTCAAGATACAGAGAGGTCTCATGCTGGTTAATCAACTCATAGATCTTGTCCAGATACAACTAGATGTATTATGACAATTAACTCAGCT | | |
| | GGGATGTGAACAAAAGTTTCCGGGATTGTGTGTTATTTCCATTCAGTATGTTAAATTTACTAGGACAGCTAATTTGTCAAAAAGTCTTTTTCAGTATATGTTAC | | |
| | AGAATTGGATGGCTGAATTTGAACAGATCCTTCGGGAATTGAGACTTCAGGTCAACTCCACGCGCTTGGACCTGTCGCTGACCAAAGGATTACCCAATTGGA | | |
| | TCTCCTCAGCATTTTCCTTCTTTAAAAAATGGGTGGGATTAATATTATTTGGAGATACACTTTGCTGTGGATTAGTGTTGCTTCTTTGATTGGTCTGTAAGCTTA | | |
| | AGGCCTAAACTAGGAGAGACAAGGTGGTTATTGCCCAGGCGCTTGCAGGACTAGAACATGGAGCTCCCCCTGATATATGGTTATCTATGCTTAGGCAATAGG | | |
| | TCGCTGGCCACTCAGCTCTTACATCTCACGAGGCTAGACTCATTGCACGGGATGGAGTGAGTGTGCTTCAGCAGCCCGAGAGAGTTGCACGGCTAAGCACTG | | |
| | CAATGGAAAGGCTCTGCGGCATATATGAGCCTATTCTAGGGAGACATGTCATCTTTCATGAAGGTTCAGTGTCCTAGTTCCCTTCCCCCAGGCAAAACGACAC | | |
| | GGGAGCAGGTCAGGGTTGCTCTGGGTAAAAGCCTGTGAGCCTAAGAGCTAATCCTGTACATGGCTCCTTTACCTACACACTGGGGATTTGACCTCTATCTCCA | | |
| | CTCTCATTAATATGGGTGGCCTATTTGCTCTTATTAAAAGAAAAGGGGGAGATGTTGGGAGCCGCGCCCACATTCGCCGTTACAAGATGGCGCTGACAGCTG | | |
| | TGTTCTAAGTGGTAAACAAATAATCTGCGCATGTGCCGAGGGTGGTTCTTCACTCTATGTGCTCTGCCTTCCCCGTGACGTCAACTCGGCCGATGGGCTGCAG | | |
| | CCAATCAGGGAGTGACACGTCCTAGGCGAAGGAGAATTCTCTTTAATAGGGACGGGGTTTTGTTTTCTCTCTCTCTTGCTTCTCGCTCGCTCTTGCTTCTTGCA | | |
| | CTCTGGCTCCTGAAGATGTAAGCAATAAAGTTTTGCCGCAGAAGATTCTGGTCTGTGGTGTTCTTCCTGGCCGGGCGTGAGAACGCGTCTAATAACA | | |

Denotations of “v1,” “v2,” and “v3” indicate alterations to the promoter/R junction (e.g., between a CMV promoter and the R U5 sequence), designed to mimic the human initiator consensus sequence (YYANWYY) without substituting base pairs. The sequences for v1, v2, and v3 variants of each transposon are shown in Tables S1-S4, as indicated. Specific modifications for each of the retrotransposons are summarized below:

### MusD6/ETnII-B3:

V1: Full length pCMV promoter with full length MusD6/ETnII-B3 R sequence.
V2: Full length pCMV promoter with 5′ truncated MusD6/ETnII-B3 R sequence.
V3: 3′ truncated pCMV promoter with full length MusD6/ETnII-B3 R sequence.

### IAP-92L23:

V1: Full length pCMV promoter with 5′ extended IAP-92L23 R sequence. The 5′ extension extends into the 3′ end of the U3 sequence.
V2: 3′ truncated pCMV promoter with full length IAP-92L23 R sequence.

### MusD Driver Plasmids:

As shown in FIG. 9A, these plasmids generally consisted of a CMV promoter driving expression of a 5′ LTR truncated MusD6 transposon followed by an SV40 polyA signal. 5′ truncations included the removal of the U3 sequence of the 5′ LTR. Notably, these vectors contained 5′ flanking sequences between the 5′ LTR and gag, including the tRNA primer binding sequence (PBS), and 3′ flanking sequences between pol and the 3′ LTR, including a polypurine tract (PPT). Driver plasmids lacking a full-length pol gene were used as a negative control to lack a capacity for reverse-transcription and integration. Pol-deficient vectors were created by inserted a stop codon after V10 of the pol gene and removed the rest of the pol gene. Drivers containing a mutant PBS were also created that prevent tRNA-priming of the transposon.

### Compact MusD Driver Plasmids:

As shown in FIG. 9B, these plasmids consisted of a CMV promoter driving expression of MusD6 gag-pro-pol followed by an SV40 polyA signal. A kozak consensus sequence was included between the CMV promoter and the gag gene. Driver plasmids lacking a full-length pol gene were used as a negative control to lack a capacity for reverse-transcription and integration. Pol-deficient vectors were created by inserted a stop codon after V10 of the pol gene and removed the rest of the pol gene.

### MusD Template Plasmids:

As shown in FIG. 9C, these plasmids consisted of a CMV promoter driving expression of a 5′ LTR truncated MusD6 transposon containing a deletion between gag and pol, followed by an SV40 polyA signal. 5′ truncations included the removal of the U3 sequence of the 5′ LTR.

The gag/pol deletion began at basepair 202 of gag and ended at basepair 2477 of pol. A heterologous object sequence was inserted at basepair 122 in the 3′ flanking region downstream the pol gene. The heterologous object sequence included a gene cassette in the antisense direction containing a EF1 alpha promoter, GFP, and a TK polyA. The GFP contained an intron that is antisense to the coding sequence of the GFP. Therefore, GFP expression only occurred after transcription, splicing and reverse-transcription of the retrotransposon.

### ETnII Template Plasmids:

These plasmids consisted of a CMV promoter driving expression of a 5′ LTR truncated ETnII-B3 transposon followed by an SV40 polyA signal. A heterologous object was inserted 2760 basepairs downstream the truncated 5′ LTR sequence. The heterologous object sequence included a gene cassette in the antisense direction containing a EF1 alpha promoter, GFP, and a TK polyA. The GFP contained an intron that was antisense to the coding sequence of the GFP. Therefore, GFP expression would only occur after transcription, splicing and reverse-transcription of the retrotransposon.

### Compact MusD Template Plasmids:

These plasmids remove the 5′ flanking sequence between the PBS and the heterologous object sequence and the 3′ flanking sequence between the heterologous object and the PPT of the MusD template plasmids.

### Compact ETnII Template Plasmids:

These plasmids remove the 5′ flanking sequence between the PBS and the heterologous object sequence and the 3′ flanking sequence between the heterologous object and the PPT of the ETnII template plasmids.

### DNA Transfection:

HEK293T cells were plated at 200,000 cells/mL at a volume of 100 μL in 96-well plates in DMEM medium supplemented with 10% fetal bovine serum and placed in a humidified incubator at 37° C. and 5% CO2. The next day, 125 ng of DNA was transfected to each well using TransIT-293 (Mirus). DNA for transfections contain combinations of MusD6 drivers and MusD6/ETnII templates in 1:1 mass ratios (56.25 ng each). A constitutively expressing BFP plasmid (pCAG-BFP) was also co-transfected (12.5 ng) with all the DNA mixtures to ensure cells were efficiently transfected. For samples that contain driver=“none” and/or template=“none”, a CMV plasmid was used that doesn't code for any proteins between the CMV promoter and the SV40 polyA was used.

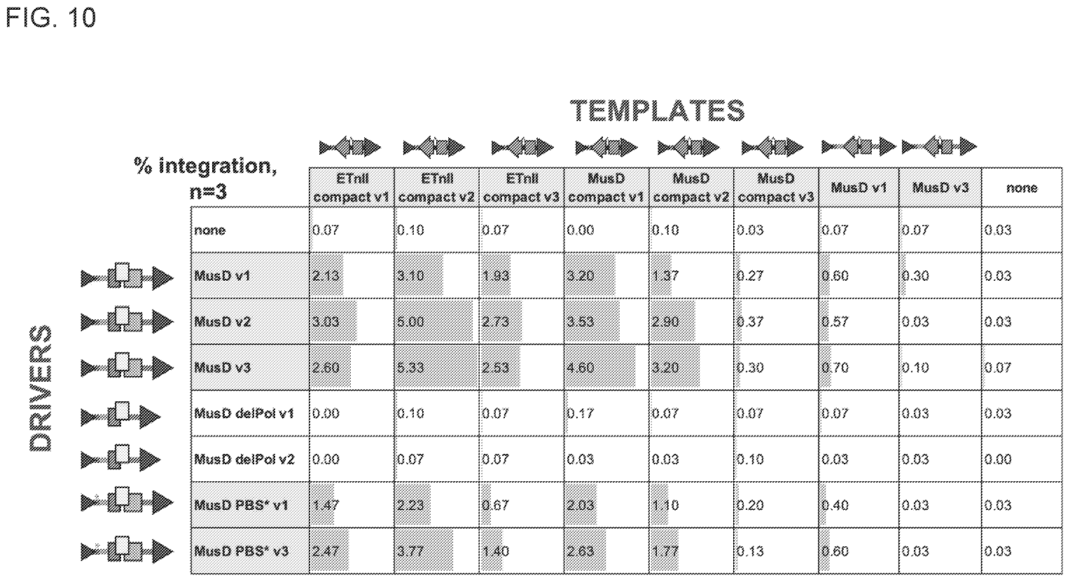

After 7 days, a digital droplet PCR instrument was used as a molecular assay to measure integration efficiencies. Genomes were extracted from the transfected or non-treated cells using flash-freezing and a Proteinase K incubation. A Taqman probe was designed to the GFP gene spanning the exon/exon junction, thus only hybridizing upon successful reverse-transcription of the post-spliced RNA template molecule. Forward and reverse primers were designed within the GFP coding sequence. The results of ddPCR copy number analysis (normalized to reference gene RPP30) are shown in FIG. 10.

### Example 8: Plasmid Delivery of an LTR Retrotransposon in Cis

This example demonstrates LTR retrotransposon-mediated integration of a genetic payload into the genome of human cells in a cis configuration. In order to assess integration, the stability of therapeutic protein expression were measured over a period of time as cells divide. Protein expression stability was conceived to occur as a result of the integration of the gene expression cassette into the human genome.

To assess expression stability, HEK293T cells were transfected with a (1) plasmid containing an active LTR retrotransposon or (2) plasmid containing an inactive LTR retrotransposon. The plasmids comprised a promoter that mediates transcription of an RNA template which comprises a promoter, an R sequence, a U5 sequence, a primer binding site (PBS) sequence, gag proteins (Matrix, Nucleocapsid, and Capsid), protease (pro) proteins, and pol proteins (Reverse transcriptase and integrase), a heterologous object sequence, polypurine tract (PPT), a 3′ LTR and an SV40 polyA sequence. The 3′LTR comprised a U3, R and U5 sequence. In this example, the 5′ R/U5 and 3′ LTR of the template RNA, and the gag, pro and pol proteins are derived from the IAP-RP23-92L23 LTR retrotransposon. The inactive retrotransposon plasmids contained either an inactivating deletion of the reverse transcriptase protein coding sequence between A14 and the stop codon, which prevents the reverse transcriptase from reverse transcribing the RNA or a mutation of the PBS, termed PBS*. A heterologous object sequence was inserted at basepair 10 in the 3′ flanking region downstream the pol gene. The heterologous object sequence included a gene cassette in the antisense direction containing a EF1 alpha promoter, GFP, and a TK polyA. The GFP contains an intron that is antisense to the coding sequence of the GFP. Therefore, GFP expression only occurred after transcription, splicing and reverse-transcription of the retrotransposon. A detailed view of these exemplary configurations is depicted in FIGS. 11A-11B. Sequences used in the exemplary constructs are listed in Tables S1-S5 above.

### DNA Transfection:

HEK293T cells were plated at 200,000 cells/mL at a volume of 100 μL in 96-well plates in DMEM medium supplemented with 10% fetal bovine serum and placed in a humidified incubator at 37° C. and 5% CO₂. The next day, 125 ng of DNA was transfected to each well using TransIT-293 (Mirus). DNA for transfections contain 104.16 ng of the retrotransposon plasmids. A constitutively expressing BFP plasmid (pCAG-BFP) was also co-transfected (20.84 ng) with all the DNA mixtures to ensure cells were efficiently transfected.

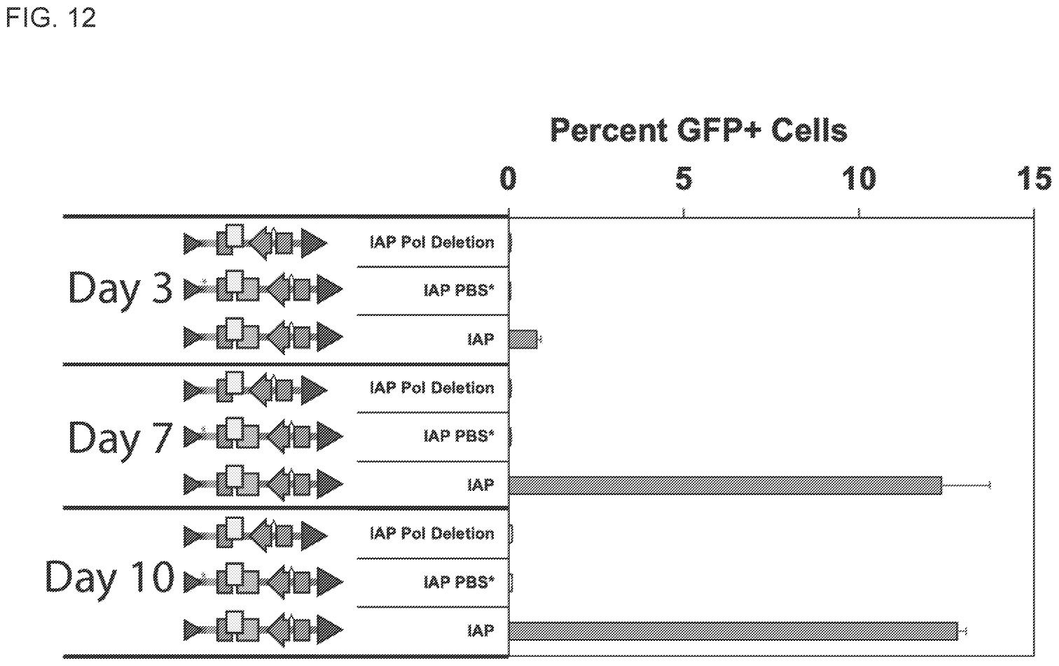

After 3, 7 and 10 days, cells were removed at various days post-transfection for flow cytometry analysis, ddPCR preparation and routine cell maintenance. Cells were resuspended in 250 μL of medium and 125 μL were transferred to a round bottom 96 well plate for cytometric analysis. An ACEA Novocyte was used to measure morphological properties (FSC/SSC) and single-cell GFP fluorescent data using a 488 nm laser and 530/30 emission filter. FlowJo (TreeStar) was used to gate for morphologically viable cells (FSC-A/SSC-A) and single cells (FSC-A, FSC-H). Next, % GFP was determined by setting an 0.1% positive gate on GFP-A of non-treated cells and applying this gate on all samples. As shown in FIG. 12, resultant percentage GFP+ cells was substantially higher for IAP than for IAP PBS* or IAP with the pol deletion. The percentage of GFP+ cells also increased substantially from day 3 to day 7 post-transfection.

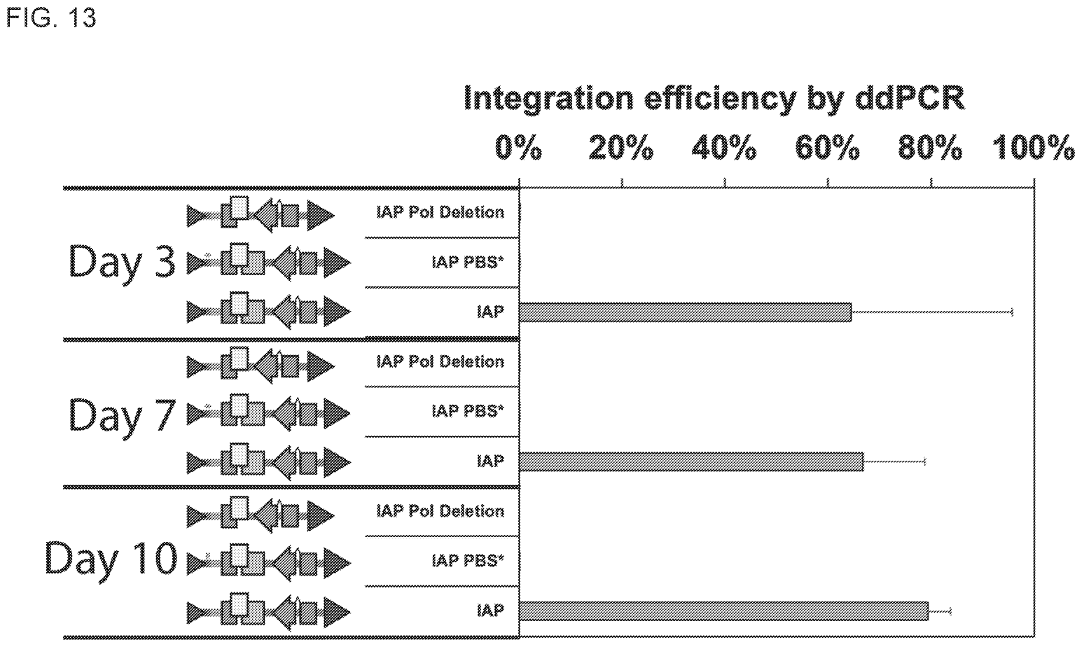

A digital droplet PCR instrument was used as a molecular assay to measure integration efficiencies. Genomes were extracted from the transfected or non-treated cells using flash-freezing and a Proteinase K incubation. A Taqman probe was designed to the GFP gene spanning the exon/exon junction, thus only hybridizing upon successful reverse-transcription of the post-spliced RNA template molecule. Forward and reverse primers were designed within the GFP coding sequence. The results of ddPCR copy number analysis (normalized to reference gene RPP30) are shown in FIG. 13. Integration efficiency was substantially higher for the IAP setting compared to IAP PBS* and IAP pol deletion at all time points tested (i.e., day 3, day 7, and day 10).

1. A system for modifying DNA comprising:
a) a template RNA comprising a first long terminal repeat (LTR), a second LTR, a heterologous object sequence encoding a therapeutic effector, positioned between the first LTR and the second LTR, and optionally a primer binding site (PBS); or a DNA molecule encoding the template RNA;
b) an LTR retrotransposon structural polypeptide domain (e.g., gag, e.g., a viral capsid (CA) protein), or a nucleic acid molecule encoding the structural polypeptide domain; and
c) an LTR retrotransposon reverse transcriptase polypeptide domain (e.g., pol) capable of reverse transcribing the template RNA, thereby producing a template DNA, or a nucleic acid molecule encoding the reverse transcriptase polypeptide domain.

2. A system for modifying DNA comprising:
a) a template RNA comprising a first LTR, a second LTR, and a heterologous object sequence encoding a therapeutic effector, positioned between the first LTR and the second LTR and optionally a primer binding site (PBS); or a DNA molecule encoding the template RNA;
b) a retroviral structural polypeptide domain (e.g., gag), or a nucleic acid molecule encoding the structural polypeptide domain;
c) a retroviral reverse transcriptase polypeptide domain (e.g., pol) capable of reverse transcribing the template RNA, thereby producing a template DNA, or a nucleic acid molecule encoding the reverse transcriptase polypeptide domain; and
the system comprises neither an envelope polypeptide domain (e.g., a retroviral envelope polypeptide domain, e.g., a lentiviral envelope polypeptide domain) nor a nucleic acid molecule encoding the envelope polypeptide domain.

3. A cell-free system for modifying DNA comprising:
a) a template RNA comprising a first LTR, a second LTR, and a heterologous object sequence encoding a therapeutic effector, positioned between the first LTR and the second LTR and optionally a primer binding site (PBS); or a DNA molecule encoding the template RNA;
b) a first RNA encoding a retroviral structural polypeptide domain (e.g., gag);
c) a second RNA encoding a retroviral reverse transcriptase polypeptide domain (e.g., pol) capable of reverse transcribing the template RNA, thereby producing a template DNA, or a nucleic acid molecule encoding the reverse transcriptase polypeptide domain; and
wherein the first RNA sequence and the second RNA sequence are optionally part of the same nucleic acid molecule.

4. A template RNA comprising:
a first retrotransposon LTR,
a second retrotransposon LTR,
a heterologous object sequence encoding a therapeutic effector, positioned between the first LTR and the second LTR, and
optionally, a primer binding site (PBS).

5. A method of delivering a heterologous object sequence to a target cell, comprising:
a) introducing into the target cell (e.g., contacting the target cell with) a template RNA comprising a first LTR, a second LTR, and a heterologous object sequence encoding a therapeutic effector, positioned between the first LTR and the second LTR, and optionally a primer binding site (PBS); and
b) introducing into the target cell (e.g., contacting the target cell with) an LTR retrotransposon structural polypeptide domain (e.g., gag), or a nucleic acid molecule encoding the structural polypeptide domain, and an LTR retrotransposon reverse transcriptase polypeptide domain (e.g., pol) capable of reverse transcribing the template RNA, thereby producing a template DNA, or a nucleic acid molecule encoding the reverse transcriptase polypeptide domain; and
c) incubating the target cell under conditions suitable for production of the template DNA.

6. A method of delivering a heterologous object sequence to a target cell, comprising:
a) introducing into the target cell (e.g., contacting the target cell with) a template RNA comprising a first LTR, a second LTR, and a heterologous object sequence encoding a therapeutic effector, positioned between the first LTR and the second LTR, and optionally a primer binding site (PBS); and
b) contacting the target cell with a first RNA encoding a retroviral structural polypeptide domain (e.g., gag) and a second RNA encoding a retroviral reverse transcriptase polypeptide domain (e.g., pol) capable of reverse transcribing the template RNA, thereby producing a template DNA, wherein the first RNA and the second RNA are optionally part of the same RNA molecule, and
c) incubating the target cell under conditions suitable for production of the template DNA.

7. A method of delivering a heterologous object sequence to a target cell, comprising:
a) introducing into the target cell (e.g., contacting the target cell with) a template RNA comprising a first LTR, a second LTR, and a heterologous object sequence encoding a therapeutic effector, positioned between the first LTR and the second LTR, and optionally a primer binding site (PBS); and
b) introducing into the target cell (e.g., contacting the target cell with) a retroviral structural polypeptide domain (e.g., gag), or a nucleic acid molecule encoding the structural polypeptide domain and a retroviral reverse transcriptase polypeptide domain (e.g., pol) capable of reverse transcribing the template RNA, thereby producing a template DNA, or a nucleic acid molecule encoding the reverse transcriptase polypeptide domain; and
c) incubating the target cell under conditions suitable for production of the template DNA;
wherein the method does not comprise introducing into the target cell either of an envelope polypeptide domain or a nucleic acid molecule encoding the envelope polypeptide domain.

8. A method of delivering a heterologous object sequence to a target cell of a patient in need thereof (e.g., in vivo or ex vivo delivery), comprising:
a) introducing into the target cell (e.g., contacting the target cell with) a template RNA comprising a first LTR, a second LTR, and a heterologous object sequence encoding a therapeutic effector, positioned between the first LTR and the second LTR, and optionally a primer binding site (PBS); and
b) contacting the target cell with a first polynucleotide encoding a retroviral structural polypeptide domain (e.g., gag), and a second polynucleotide encoding retroviral reverse transcriptase polypeptide domain (e.g., pol) capable of reverse transcribing the template RNA, thereby producing a template DNA, wherein the first polynucleotide and the second polynucleotide are optionally part of the same polynucleotide molecule; and
c) incubating the target cell under conditions suitable for production of the template DNA.
